SEQUENCE LISTING (2 sequences)

SEQ ID NO: 1 - protein, 298 aa, Homo sapiens
MENFQKVEKIGEGTYGVVYKARNKLTGEVVALKKIRLDTETEGVPSTAIREISLLKELNHPNIVKLLDVIHTENKLYLVFEFLHQDLKKFMDASALTGIPLPLIKSYLFQLLQGLAFCHSHRVLHRDLKPQNLLINTEGAIKLADFGLARAFGVPVRTYTHEVVTLWYRAPEILLGCKYYSTAVDIWSLGCIFAEMVTRRALFPGDSEIDQLFRIFRTLGTPDEVVWPGVTSMPDYKPSFPKWARQDFSKVVPPLDEDGRSLLSQMLHYDPNKRISAKAALAHPFFQDVTKPVPHLRL

SEQ ID NO: 2 - protein, 264 aa, Homo sapiens
MENFQKVEKIGEGTYGVVYKARNKLTGEVVALKKIRLDTETEGVPSTAIREISLLKELNHPNIVKLLDVIHTENKLYLVFEFLHQDLKKFMDASALTGIPLPLIKSYLFQLLQGLAFCHSHRVLHRDLKPQNLLINTEGAIKLADFGLARAFGVPVRTYTHEVTRRALFPGDSEIDQLFRIFRTLGTPDEVVWPGVTSMPDYKPSFPKWARQDFSKVVPPLDEDGRSLLSQMLHYDPNKRISAKAALAHPFFQDVTKPVPHLRL

(19) United States
(12) Patent Application Publication
(10) Pub. No.: US 2026/0308147 A1
(43) Pub. Date: Oct. 8, 2026

(54) SUBSTITUTED OXOISOINDOLINYL PIPERIDINE-2,6-DIONE COMPOUNDS AND AZA ANALOGS THEREOF

(71) Applicant: BRISTOL-MYERS SQUIBB COMPANY, Princeton, NJ (US)

(72) Inventors: ALEXANDRE BELIN, TOULOUSE CEDEX (FR); FREDERIC CACHOUX, TOULOUSE CEDEX (FR); OLGA GHERBOVET, TOULOUSE CEDEX (FR); AURELIEN HONRAEDT, TOULOUSE CEDEX (FR); THOMAS PANCALDI, TOULOUSE CEDEX (FR); JEROME SARIE, TOULOUSE CEDEX (FR); PATRICIA WALTER, TOULOUSE CEDEX (FR); PETER KINAM PARK, NEW YORK, NY (US)

(21) Appl. No.: 19/574,893

(22) Filed: Mar. 23, 2026

Related U.S. Application Data
(60) Provisional application No. 63/776,333, filed on Mar. 24, 2025.

(51) Int. Cl. C07D 401/14; A61K 31/454; A61K 31/4545; A61K 31/4709; A61K 31/4985; A61K 31/5377; A61K 31/538; A61K 31/5386; A61K 31/55; A61K 31/553; A61K 31/69; A61K 45/06; A61P 35/00; C07B 59/00; C07D 405/14; C07D 413/14; C07D 417/14; C07D 471/04; C07D 487/04; C07D 498/08; C07F 5/02

(52) U.S. Cl. CPC C07D 401/14; A61K 31/454; A61K 31/4545; A61K 31/4709; A61K 31/4985; A61K 31/5377; A61K 31/538; A61K 31/5386; A61K 31/55; A61K 31/553; A61K 31/69; A61K 45/06; A61P 35/00; C07B 59/002; C07D 405/14; C07D 413/14; C07D 417/14; C07D 471/04; C07D 487/04; C07D 498/08; C07F 5/025

(57) ABSTRACT

Disclosed are compounds of Formula (I):

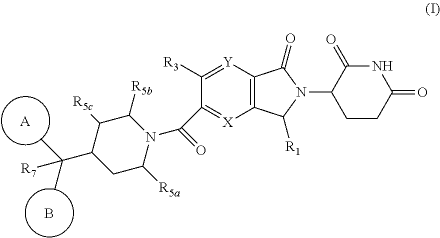

or stereoisomers, tautomers, or salts thereof, wherein Ring A, Ring B, Y, X, R₁, R₃, R₅ₐ, R_{5b}, R_{5c}, and R₇ are defined herein. Also disclosed are methods of using such compounds to decrease the level of CDK2 protein; and pharmaceutical compositions comprising such compounds. These compounds are useful in the treatment of proliferative disorders, such as cancer.

### CROSS-REFERENCE TO RELATED APPLICATIONS

This application claims the priority benefit of U.S. Provisional Application No. 63/776,333, filed Mar. 24, 2025; the content of which is herein incorporated by reference in its entirety.

### REFERENCE TO SEQUENCE LISTING SUBMITTED ELECTRONICALLY VIA PATENT CENTER

Incorporated herein by reference in its entirety is a Sequence Listing entitled, “20260519_SEQ LISTING_14500-US-NP” comprising SEQ ID NO: 1 through SEQ ID NO: 2, which include nucleic acid and/or amino acid sequences disclosed herein. The Sequence Listing has been submitted herein in XML format via Patent Center and thus constitutes both the paper and computer readable form thereof. The Sequence Listing was first created using WIPO Sequence on May 19, 2026, and is 3,352 bytes.

### DESCRIPTION

The present invention generally relates to substituted oxoisoindolinyl piperidine-2,6-dione compounds and aza analogs thereof useful for decreasing the levels of the CDK2 protein. Provided herein are substituted oxoisoindolinyl piperidine-2,6-dione compounds and aza analogs thereof, compositions comprising such compounds, and methods of their use. The invention further pertains to pharmaceutical compositions comprising at least one compound according to the invention that are useful for the treatment of proliferative disorders, such as cancer.

### BACKGROUND OF THE INVENTION

Cyclin-dependent kinase 2 (CDK2) is a critical enzyme involved in the regulation of the cell cycle, particularly the transition from the G1 phase to the S phase (Ding L, C. J. (2020). The roles of cyclin-dependent kinases in cell-cycle progression and therapeutic strategies in human breast cancer. Int J Mol Sci., 21:1960); Caruso J A, D. M. (2018). Low-molecular-weight cyclin E in human cancer: cellular consequences and opportunities for targeted therapies. Cancer Res., 78:5481-5491). For example, cyclin E/CDK2 mediates the phosphorylation of Retinoblastoma protein (RB1), resulting in its dissociation from E2F transcription factors that activate the transcription of a suite of genes involved in cell cycle progression, which triggers the transition to S-phase (Ding L, C. J. (2020). Int J Mol Sci., 21:1960). Additionally, activation of cyclin A/CDK2 during S-phase is critical for ensuring DNA replication and S-phase completion, via RB1-independent mechanisms (Ding L, C. J. (2020). Int J Mol Sci., 21:1960). Dysregulation of CDK2 activity has been implicated in the uncontrolled proliferation of cancer cells (Tadesse S, A. A. (2020). Targeting CDK2 in cancer: challenges and opportunities for therapy. Drug Discov Today, 25:406-413; Caruso J A, D. M. (2018). Cancer Res., 78:5481-5491). For example, CDK2 dependency has been shown to be linked to CCNE amplification/overexpression in cancer. CDK2 dependency has also been described in cancer bearing RB1 loss and in breast cancers resistant to CDK4/6 inhibitors (Wang, Y. (2023). Phase 1/2 study of ARTS-021, a potent, oral administrated, selective CDK2 inhibitor, in advanced or metastatic solid tumors. J Clin Oncol, 41 (16_suppl): e17546-e17546; Caruso J A, D. M. (2018). Cancer Res., 78:5481-5491); German B, A. S. (2024). MYBL2 Drives Prostate Cancer Plasticity: Inhibiting Its Transcriptional Target CDK2 for RB1-Deficient Neuroendocrine Prostate Cancer. Cancer Res Commun, 4 (9): 2295-2307).

Traditional inhibitors of CDK2 have shown limited efficacy due to compensatory mechanisms. Moreover, off-target effects have led to adverse effects in clinical trials. For example, relatively limited selectivity over other kinases and ability to inhibit only active or inactive forms of CDK2 are described limitations of CDK2 inhibitor approach (Arora M, M. J. (2023). Rapid adaptation to CDK2 inhibition exposes intrinsic cell-cycle plasticity. Cell, 186 (12): 2628-2643.e21; Tadesse S, A. A. (2020). Targeting CDK2 in cancer: challenges and opportunities for therapy. Drug Discov Today, 25:406-413; Kumarasamy V, W. J. (2025). Discrete vulnerability to pharmacological CDK2 inhibition is governed by heterogeneity of the cancer cell cycle. Nat Commun., 16 (1): 1476).

There remains a need for therapies that can decrease the levels of CDK2 protein.

There also remains a need for therapies that can decrease the levels of CDK2 protein and are selective over GSPT1.

The present invention fills the foregoing need by providing compounds that are useful to decrease the level of CDK2 protein.

The present invention fills the foregoing need by providing compounds that are useful to decrease the level of CDK2 and are selective against degradation of GSPT1.

### SUMMARY OF THE INVENTION

The present invention provides substituted oxoisoindolinyl piperidine-2,6-dione compounds and aza analogs of Formula (I), including stereoisomers, tautomers, salts, and prodrugs thereof, which are useful to decrease the levels of the CDK2.

The present invention also provides pharmaceutical compositions comprising a compound of Formula (I), stereoisomers, tautomers, pharmaceutically acceptable salts, or prodrugs thereof; and a pharmaceutically acceptable carrier.

The present invention also provides a method of treating a disease or disorder by decreasing the levels of the CDK2, the method comprising administering to a patient a compound of Formula (I), stereoisomers, tautomers, pharmaceutically acceptable salts, or prodrugs thereof.

The present invention also provides processes and intermediates for making the compounds of Formula (I), stereoisomers, tautomers, or salts thereof.

The present invention also provides the use of the compounds of Formula (I), or stereoisomers, tautomers, pharmaceutically acceptable salts, or prodrugs thereof, for the manufacture of a medicament to decrease CDK2 protein levels, for the treatment of certain diseases, including cancer.

The compounds of Formula (I) and compositions comprising the compounds of Formula (I) may be used in treating, preventing, or curing various proliferative disorders, such as cancer. Pharmaceutical compositions comprising these compounds are useful in treating, preventing, or slowing the progression of diseases or disorders in a variety of therapeutic areas, such as cancer.

These and other features of the invention will be set forth in expanded form as the disclosure continues.

### DETAILED DESCRIPTION

Applicants have found substituted oxoisoindolinyl piperidine-2,6-dione compounds and aza analogs thereof that decrease the levels of CDK2 protein. The substituted oxoisoindolinyl piperidine-2,6-dione compounds and aza analogs are believed to facilitate the interaction of CDK2 protein with the corresponding E3 ubiquitin ligase complex (Cullin4-Cereblon, CUL4-CRBN), with concomitant degradation of the CDK2 protein. These compounds are useful for the treatment of certain diseases, including cancer. The compounds are provided to be useful as pharmaceuticals with desirable stability, bioavailability, therapeutic index, and toxicity values that are important to their druggability.

The first aspect of the present invention provides at least one compound of Formula (I):

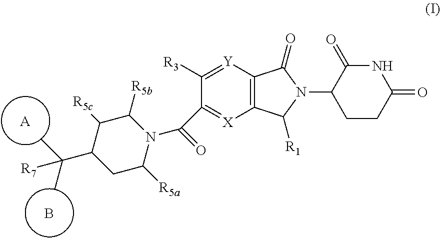

or stereoisomers, tautomers, or salts thereof, wherein:
(i) X is CR₂ and Y is CR₄;
(ii) X is CR₂ and Y is N;
(iii) X is N and Y is N;
R₁ is hydrogen or —CH₃;
R₂ is hydrogen or —CH₃;
R₃ is hydrogen, F, —CH₃, —CHF₂, or —CH₂N(CH₃)₂;
R₄ is hydrogen;
wherein:
(i) R₅ₐ, R_{5b}, and R_{5c} are each hydrogen;
(ii) R₅ₐ and R_{5b} are joined together to form —CH₂CH₂-or —CH₂OCH₂—, and R_{5c} is hydrogen; or
(iii) R₅ₐ and R_{5c} are joined together to form —CH₂—, and R_{5b} is hydrogen; Ring A is:

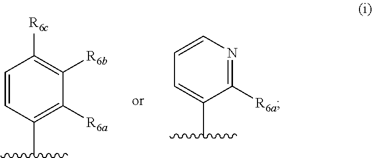

or
(ii) indazolyl, indolinyl, dihydrobenzofuranyl, dihydrobenzoxazinyl, tetrahydroquinolinyl, tetrahydrobenzazepinyl, or tetrahydrobenzooxazepinyl;
Ring B is phenyl, isothiazolyl,

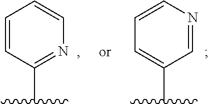 [SMILES: *c1ccccn1 ; *c1cccnc1]

R₆ₐ is hydrogen, Cl, or Br;
R_{6b} is hydrogen, —OH, —OCH₃, —OCD₃, —OCH₂CH₃, —OCH(CH₃)₂, —OCH₂CH₂OH, —OCH₂CH₂OCH₃, —OCF₃, —OCH₂CH₂F, —OCH₂CH₂NH₂, —OCH₂CH₂NH(CH₃), —OCH₂CH₂N(CH₃)₂, —OCH₂CH₂CH₂NH₂, —OCH₂CH₂CH₂N(CH₃)₂, —OCH₂CHFCH₂NH₂, —OCH₂CF₂CH₂NH₂, —OCH(CH₃)CH₂NH₂, —OCH₂CH(CH(CH₃)₂)NH₂, —OCH₂CH(CH₃)NH₂, —OCH₂CH(CH₂CH₃)NH₂, —OCH₂CH(CHF₂)NH₂, —OCH₂CH(CF₃)NH₂, —OCH₂CH(CH₂CHF₂)NH₂, —OCH₂CH₂NHC(O)CH₃, —B(OH)₂, —CH₂CH₂N(CH₃)₂, —NH(CH₃), —N(CH₃)₂, —NH(CH₂CH₂OH), —NH(CH₂CH₂NH₂), —NH(aminocyclobutyl), —NHC(O)CH₃, —NHC(O)CH₂OH, —O(cyclopropyl), —O(cyclobutyl), —O(aminocyclopentyl), —O(methylazetidinyl), —O(methyl pyrrolidinyl), —O(fluoroethyl pyrrolidinyl), —O(trifluoroethyl pyrrolidinyl), —O(methyl azaspiro[3.3]heptanyl), —OCH₂(aminocyclopropyl), —OCH₂(imidazolyl), —OCH₂(methyl imidazolyl), —OCH₂(isoxazolyl), —OCH₂(oxazolyl), —OCH₂(methyl pyrrolidinyl), —OCH₂CH₂(pyrrolidinyl), morpholinyl, oxazolyl, aminopyrrolidinyl, hydroxypyrrolidinyl, or dihydroimidazo[1,5-a]pyrazinyl;
R_{6c} is hydrogen or F; and
R₇ is hydrogen or deuterium.

One embodiment provides a compound of Formula (I), or stereoisomers, tautomers, or pharmaceutically acceptable salts thereof.

One embodiment provides a compound of Formula (I), or stereoisomers or tautomers thereof.

One embodiment provides a salt of the compound of Formula (I), or stereoisomers or tautomers thereof.

One embodiment provides a pharmaceutically acceptable salt of the compound of Formula (I), or stereoisomers or tautomers thereof.

One embodiment provides a compounds of Formula (I) having the structure:

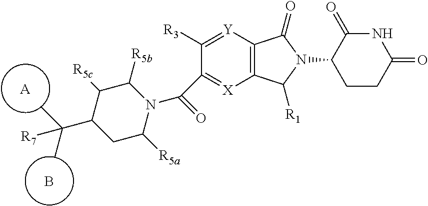

or tautomers or salts thereof.

One embodiment provides a compounds of Formula (I) having the structure:

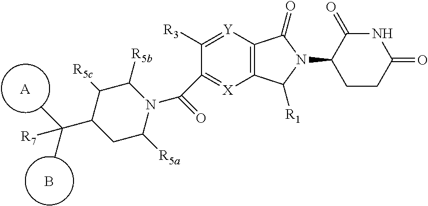

or tautomers or salts thereof.

One embodiment provides a compounds of Formula (I) having the structure:

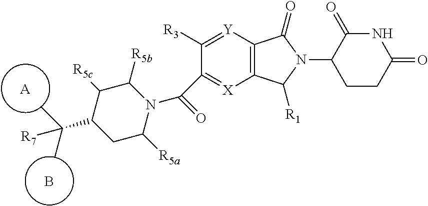

or tautomers or salts thereof.

One embodiment provides a compounds of Formula (I) having the structure:

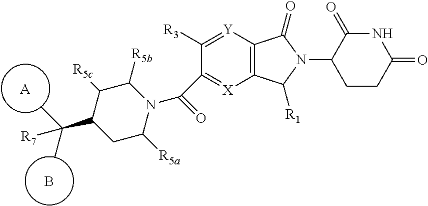

or tautomers or salts thereof.

One embodiment provides a compounds of Formula (I) having the structure:

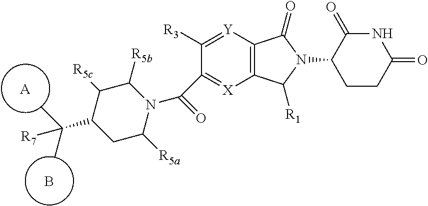

or tautomers or salts thereof.

One embodiment provides a compounds of Formula (I) having the structure:

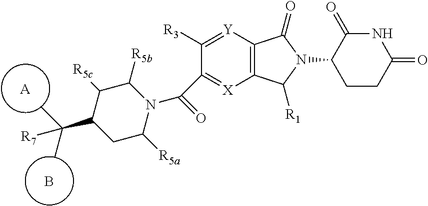

or tautomers or salts thereof.

One embodiment provides a compounds of Formula (I) having the structure:

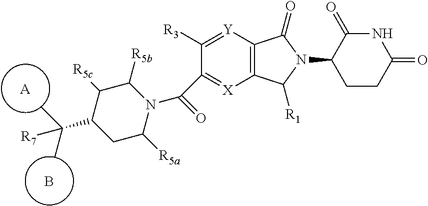

or tautomers or salts thereof.

One embodiment provides a compound of Formula (I) or stereoisomers, tautomers, or salts thereof, wherein X is CR₂ and Y is CR₄. Compounds of this embodiment have the structure of Formula (II):

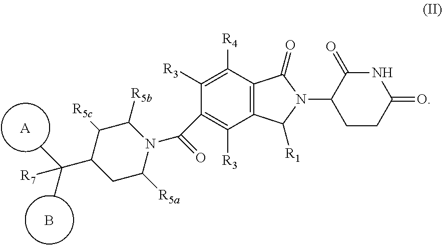

One embodiment provides a compound of Formula (I) or stereoisomers, tautomers, or salts thereof, wherein X is CR₂ and Y is N. Compounds of this embodiment have the structure of Formula (III):

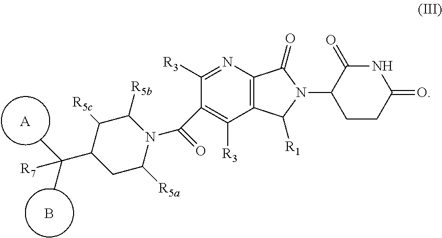

One embodiment provides a compound of Formula (I) or stereoisomers, tautomers, or salts thereof, wherein X is N and Y is N. Compounds of this embodiment have the structure of Formula (IV):

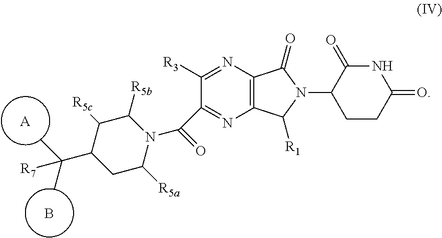

One embodiment provides a compound of Formula (I) or stereoisomers, tautomers, or salts thereof, wherein R₁ is H.

One embodiment provides a compound of Formula (I) or stereoisomers, tautomers, or salts thereof, wherein R₁ is-CH₃.

One embodiment provides a compound of Formula (I) or stereoisomers, tautomers, or salts thereof, wherein X is CR₂; Y is CR₄ or N; R₂ is hydrogen or —CH₃; and R₄ is hydrogen. Include in this embodiment are compounds in which R₂ is hydrogen. Also included in this embodiment are compounds in which R₂ is-CH₃.

One embodiment provides a compound of Formula (I) or stereoisomers, tautomers, or salts thereof, wherein X is CR₂ or N; Y is N; R₂ is hydrogen or —CH₃; and R₄ is hydrogen.

One embodiment provides a compound of Formula (I) or stereoisomers, tautomers, or salts thereof, wherein R₃ is hydrogen.

One embodiment provides a compound of Formula (I) or stereoisomers, tautomers, or salts thereof, wherein R₃ is F, —CH₃, —CHF₂, or —CH₂N(CH₃)₂.

One embodiment provides a compound of Formula (I) or stereoisomers, tautomers, or salts thereof, wherein R₃ is F, —CH₃, or —CHF₂.

One embodiment provides a compound of Formula (I) or stereoisomers, tautomers, or salts thereof, wherein R₃ is-CH₃ or —CHF₂.

One embodiment provides a compound of Formula (I) or stereoisomers, tautomers, or salts thereof, wherein R₃ is-CH₃.

One embodiment provides a compound of Formula (I) or stereoisomers, tautomers, or salts thereof, wherein R₅ₐ, R_{5b}, and R_{5c} are each hydrogen. Compounds of this embodiment have the structure of Formula (Ia):

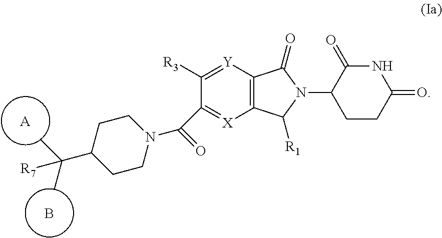

One embodiment provides a compound of Formula (I) or stereoisomers, tautomers, or salts thereof, wherein R₅ₐ and R_{5b} are joined together to form —CH₂CH₂-and R_{5c} is hydrogen. Compounds of this embodiment have the structure of Formula (Ib):

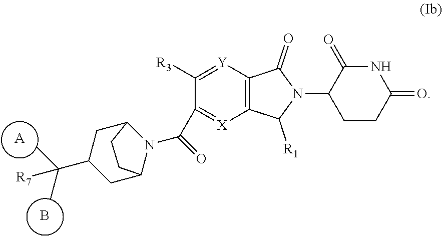

One embodiment provides a compound of Formula (I) or stereoisomers, tautomers, or salts thereof, wherein R₅ₐ and R_{5b} are joined together to form —CH₂OCH₂—, and R_{5c} is hydrogen. Compounds of this embodiment have the structure of Formula (Ic):

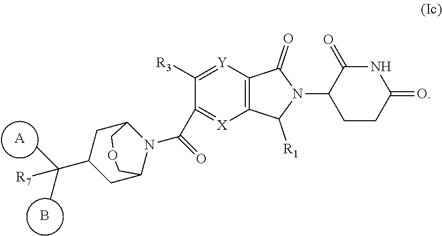

One embodiment provides a compound of Formula (I) or stereoisomers, tautomers, or salts thereof, wherein R₅ₐ and R_{5c} are joined together to form —CH₂—, and R_{5b} is hydrogen. Compounds of this embodiment have the structure of Formula (ID):

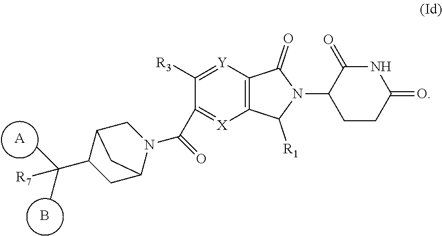

One embodiment provides a compound of Formula (I) or stereoisomers,

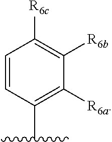

tautomers, or salts thereof, wherein Ring A is Included in this embodiment are compounds in which R₆ₐ is Cl. Also included in this embodiment are compounds in which R₆ₐ is Cl, R_{6b} is hydrogen, and Roc is hydrogen.

One embodiment provides a compound of Formula (I) or stereoisomers, tautomers, or salts thereof, wherein Ring A is

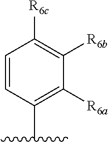

and R_{6b} is hydrogen or —OCH₃. Included in this embodiment are compounds in which R₆ₐ is Cl and R_{6c} is hydrogen.

One embodiment provides a compound of Formula (I) or stereoisomers, tautomers, or salts thereof, wherein Ring A is

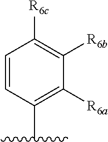

and R_{6b} is-OH, —OCH₃, —OCD₃, —OCH₂CH₃, —OCH(CH₃)₂, —OCH₂CH₂OH, —OCH₂CH₂OCH₃, —OCF₃, —OCH₂CH₂F, —OCH₂CH₂NH₂, —OCH₂CH₂NH(CH₃), —OCH₂CH₂N(CH₃)₂, —OCH₂CH₂CH₂NH₂, —OCH₂CH₂CH₂N(CH₃)₂, —OCH₂CHFCH₂NH₂, —OCH₂CF₂CH₂NH₂, —OCH(CH₃)CH₂NH₂, —OCH₂CH(CH(CH₃)₂)NH₂, —OCH₂CH(CH₃)NH₂, —OCH₂CH(CH₂CH₃)NH₂, —OCH₂CH(CHF₂)NH₂, —OCH₂CH(CF₃)NH₂, —OCH₂CH(CH₂CHF₂)NH₂, —OCH₂CH₂NHC(O)CH₃, —B(OH)₂, —CH₂CH₂N(CH₃)₂, —NH(CH₃), —N(CH₃)₂, —NH(CH₂CH₂OH), —NH(CH₂CH₂NH₂), —NH(aminocyclobutyl), —NHC(O)CH₃, —NHC(O)CH₂OH, —O(cyclopropyl), —O(cyclobutyl), —O(aminocyclopentyl), —O(methylazetidinyl), —O(methyl pyrrolidinyl), —O(fluoroethyl pyrrolidinyl), —O(trifluoroethyl pyrrolidinyl), —O(methyl azaspiro[3.3]heptanyl), —OCH₂(aminocyclopropyl), —OCH₂(imidazolyl), —OCH₂(methyl imidazolyl), —OCH₂(isoxazolyl), —OCH₂(oxazolyl), —OCH₂(methyl pyrrolidinyl), —OCH₂CH₂(pyrrolidinyl), morpholinyl, oxazolyl, aminopyrrolidinyl, hydroxypyrrolidinyl, or dihydroimidazo[1,5-a]pyrazinyl. Included in this embodiment are compounds in which R₆ₐ is Cl and Roc is hydrogen.

One embodiment provides a compound of Formula (I) or stereoisomers, tautomers, or salts thereof, wherein Ring A is

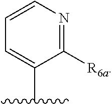

Included in this embodiment are compounds in which R₆ₐ is Cl.

One embodiment provides a compound of Formula (I) or stereoisomers, tautomers, or salts thereof, wherein Ring A is indazolyl, indolinyl, dihydrobenzofuranyl, dihydrobenzoxazinyl, tetrahydroquinolinyl, tetrahydrobenzazepinyl, or tetrahydrobenzooxazepinyl.

One embodiment provides a compound of Formula (I) or stereoisomers, tautomers, or salts thereof, wherein Ring A is

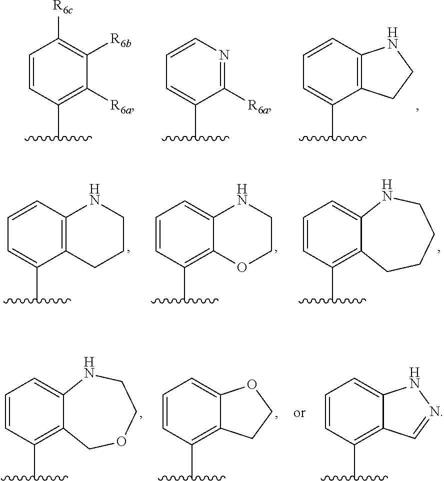

One embodiment provides a compound of Formula (I) or stereoisomers, tautomers, or salts thereof, wherein Ring A is

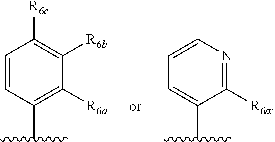

One embodiment provides a compound of Formula (I) or stereoisomers, tautomers, or salts thereof, wherein Ring A is

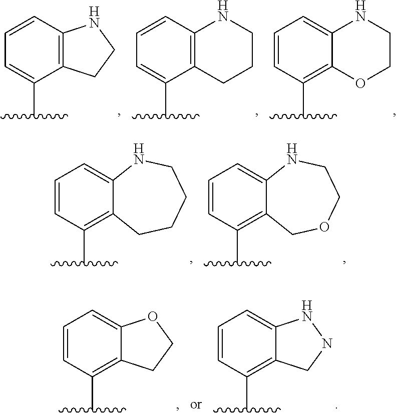

One embodiment provides a compound of Formula (I) or stereoisomers, tautomers, or salts thereof, wherein Ring B is phenyl, isothiazolyl, or

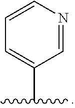 [SMILES: *c1cccnc1]

One embodiment provides a compound of Formula (I) or stereoisomers, tautomers, or salts thereof, wherein Ring B is phenyl.

One embodiment provides a compound of Formula (I) or stereoisomers, tautomers, or salts thereof, wherein Ring B is isothiazolyl.

One embodiment provides a compound of Formula (I) or stereoisomers, tautomers, or salts thereof, wherein Ring B is

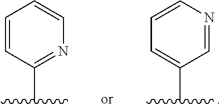 [SMILES: *c1ccccn1 ; *c1cccnc1]

One embodiment provides a compound of Formula (I) or stereoisomers, tautomers, or salts thereof, wherein Ring B is

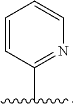 [SMILES: *c1ccccn1]

One embodiment provides a compound of Formula (I) or stereoisomers, tautomers, or salts thereof, wherein Ring B is

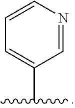 [SMILES: *c1cccnc1]

One embodiment provides a compound of Formula (I) or stereoisomers, tautomers, or salts thereof, wherein Ring A is

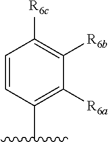

and Ring B is phenyl. Included in this embodiment are compounds in which R₆ₐ is Cl.

One embodiment provides a compound of Formula (I) or stereoisomers, tautomers, or salts thereof, wherein X is CH; Y is N; Ring A is

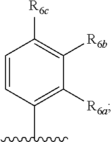

Ring B is phenyl; R₁ is hydrogen; R₂ is hydrogen; R₃ is hydrogen or —CH₃; R₅ₐ is hydrogen; R_{5b} is hydrogen; R_{5c} is hydrogen; R₆ₐ is Cl; R_{6b} is hydrogen or —OCH₃; and R_{6c} is hydrogen.

One embodiment provides a compound of Formula (I), or stereoisomers, tautomers, or salts thereof, wherein said compound is:

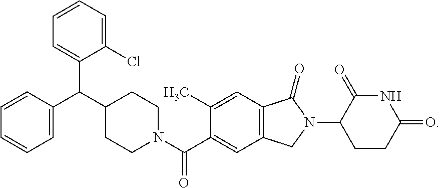 [SMILES: Cc1cc2c(cc1C(=O)N1CCC(C(c3ccccc3)c3ccccc3Cl)CC1)CN(C1CCC(=O)NC1=O)C2=O]

One embodiment provides a compound of Formula (I), or stereoisomers, tautomers, or salts thereof, wherein said compound is:

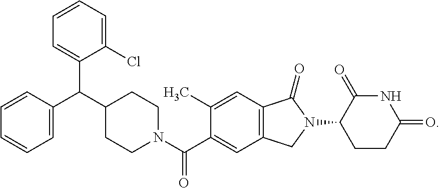 [SMILES: Cc1cc2c(cc1C(=O)N1CCC(C(c3ccccc3)c3ccccc3Cl)CC1)CN([C@H]1CCC(=O)NC1=O)C2=O]

One embodiment provides a compound of Formula (I), or stereoisomers, tautomers, or salts thereof, wherein said compound is:

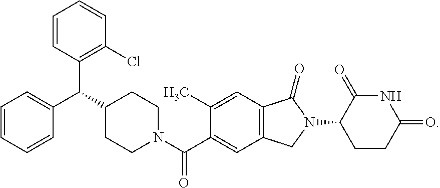 [SMILES: Cc1cc2c(cc1C(=O)N1CCC([C@H](c3ccccc3)c3ccccc3Cl)CC1)CN([C@H]1CCC(=O)NC1=O)C2=O]

One embodiment provides a compound of Formula (I), or stereoisomers, tautomers, or salts thereof, wherein said compound is:

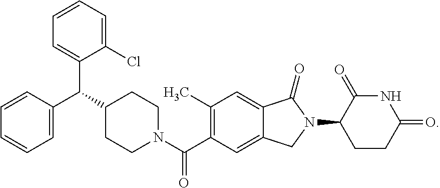 [SMILES: Cc1cc2c(cc1C(=O)N1CCC([C@H](c3ccccc3)c3ccccc3Cl)CC1)CN([C@@H]1CCC(=O)NC1=O)C2=O]

One embodiment provides a compound of Formula (I), or stereoisomers, tautomers, or salts thereof, wherein said compound is:

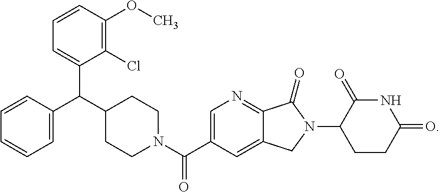 [SMILES: COc1cccc(C(c2ccccc2)C2CCN(C(=O)c3cnc4c(c3)CN(C3CCC(=O)NC3=O)C4=O)CC2)c1Cl]

One embodiment provides a compound of Formula (I), or stereoisomers, tautomers, or salts thereof, wherein said compound is:

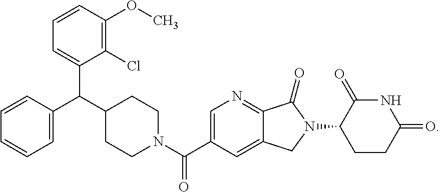 [SMILES: COc1cccc(C(c2ccccc2)C2CCN(C(=O)c3cnc4c(c3)CN([C@H]3CCC(=O)NC3=O)C4=O)CC2)c1Cl]

One embodiment provides a compound of Formula (I), or stereoisomers, tautomers, or salts thereof, wherein said compound is:

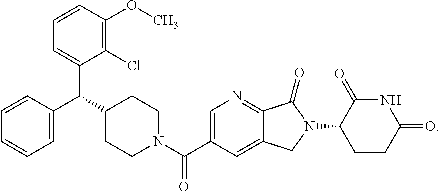 [SMILES: COc1cccc([C@@H](c2ccccc2)C2CCN(C(=O)c3cnc4c(c3)CN([C@H]3CCC(=O)NC3=O)C4=O)CC2)c1Cl]

One embodiment provides a compound of Formula (I), or stereoisomers, tautomers, or salts thereof, wherein said compound is:

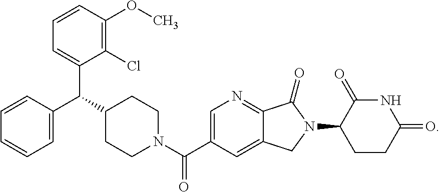 [SMILES: COc1cccc([C@@H](c2ccccc2)C2CCN(C(=O)c3cnc4c(c3)CN([C@@H]3CCC(=O)NC3=O)C4=O)CC2)c1Cl]

One embodiment provides a compound of Formula (I), or stereoisomers, tautomers, or salts thereof, wherein said compound is:

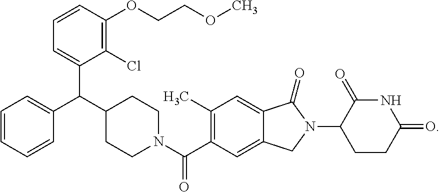 [SMILES: COCCOc1cccc(C(c2ccccc2)C2CCN(C(=O)c3cc4c(cc3C)C(=O)N(C3CCC(=O)NC3=O)C4)CC2)c1Cl]

One embodiment provides a compound of Formula (I), or stereoisomers, tautomers, or salts thereof, wherein said compound is:

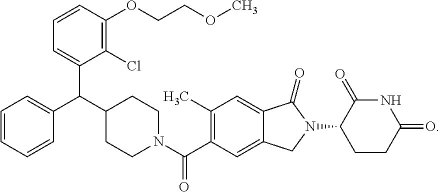 [SMILES: COCCOc1cccc(C(c2ccccc2)C2CCN(C(=O)c3cc4c(cc3C)C(=O)N([C@H]3CCC(=O)NC3=O)C4)CC2)c1Cl]

One embodiment provides a compound of Formula (I), or stereoisomers, tautomers, or salts thereof, wherein said compound is:

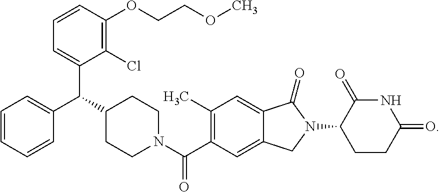 [SMILES: COCCOc1cccc([C@@H](c2ccccc2)C2CCN(C(=O)c3cc4c(cc3C)C(=O)N([C@H]3CCC(=O)NC3=O)C4)CC2)c1Cl]

One embodiment provides a compound of Formula (I), or stereoisomers, tautomers, or salts thereof, wherein said compound is:

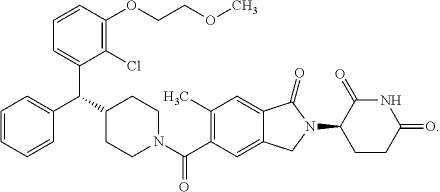 [SMILES: COCCOc1cccc([C@@H](c2ccccc2)C2CCN(C(=O)c3cc4c(cc3C)C(=O)N([C@@H]3CCC(=O)NC3=O)C4)CC2)c1Cl]

One embodiment provides a compound of Formula (I), or stereoisomers, tautomers, or salts thereof, wherein said compound is:

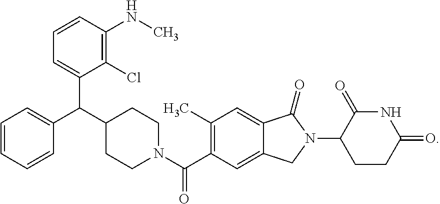 [SMILES: CNc1cccc(C(c2ccccc2)C2CCN(C(=O)c3cc4c(cc3C)C(=O)N(C3CCC(=O)NC3=O)C4)CC2)c1Cl]

One embodiment provides a compound of Formula (I), or stereoisomers, tautomers, or salts thereof, wherein said compound is:

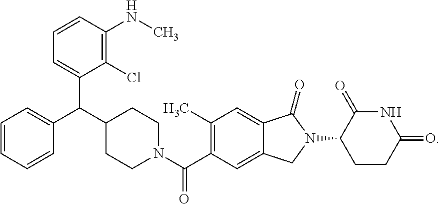 [SMILES: CNc1cccc(C(c2ccccc2)C2CCN(C(=O)c3cc4c(cc3C)C(=O)N([C@H]3CCC(=O)NC3=O)C4)CC2)c1Cl]

One embodiment provides a compound of Formula (I), or stereoisomers, tautomers, or salts thereof, wherein said compound is:

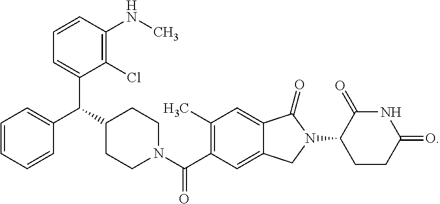 [SMILES: CNc1cccc([C@@H](c2ccccc2)C2CCN(C(=O)c3cc4c(cc3C)C(=O)N([C@H]3CCC(=O)NC3=O)C4)CC2)c1Cl]

One embodiment provides a compound of Formula (I), or stereoisomers, tautomers, or salts thereof, wherein said compound is:

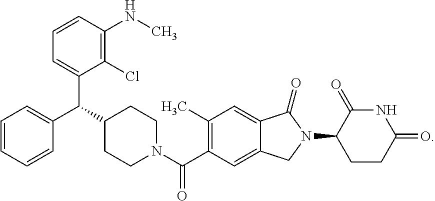 [SMILES: CNc1cccc([C@@H](c2ccccc2)C2CCN(C(=O)c3cc4c(cc3C)C(=O)N([C@@H]3CCC(=O)NC3=O)C4)CC2)c1Cl]

One embodiment provides a compound of Formula (I), or stereoisomers, tautomers, or salts thereof, wherein said compound is:

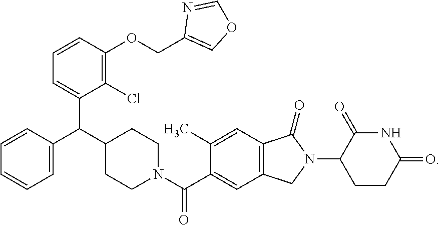 [SMILES: Cc1cc2c(cc1C(=O)N1CCC(C(c3ccccc3)c3cccc(OCc4cocn4)c3Cl)CC1)CN(C1CCC(=O)NC1=O)C2=O]

One embodiment provides a compound of Formula (I), or stereoisomers, tautomers, or salts thereof, wherein said compound is:

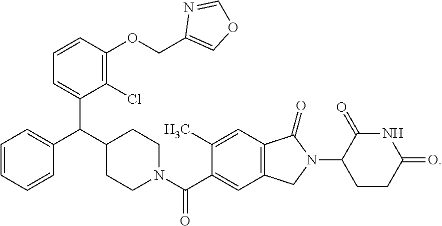 [SMILES: Cc1cc2c(cc1C(=O)N1CCC(C(c3ccccc3)c3cccc(OCc4cocn4)c3Cl)CC1)CN(C1CCC(=O)NC1=O)C2=O]

One embodiment provides a compound of Formula (I), or stereoisomers, tautomers, or salts thereof, wherein said compound is:

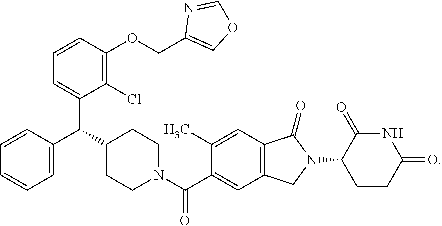 [SMILES: Cc1cc2c(cc1C(=O)N1CCC([C@H](c3ccccc3)c3cccc(OCc4cocn4)c3Cl)CC1)CN([C@H]1CCC(=O)NC1=O)C2=O]

One embodiment provides a compound of Formula (I), or stereoisomers, tautomers, or salts thereof, wherein said compound is:

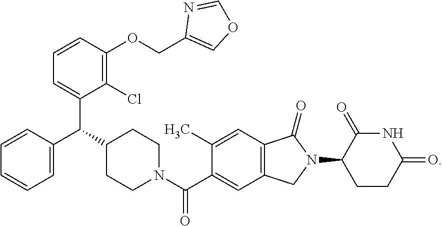 [SMILES: Cc1cc2c(cc1C(=O)N1CCC([C@H](c3ccccc3)c3cccc(OCc4cocn4)c3Cl)CC1)CN([C@@H]1CCC(=O)NC1=O)C2=O]

One embodiment provides a compound of Formula (I), or stereoisomers, tautomers, or salts thereof, wherein said compound is:
(3S)-3-[5-(4-benzhydrylpiperidine-1-carbonyl)-1-oxo-isoindolin-2-yl]piperidine-2,6-dione (1);
(3S)-3-[5-(4-benzhydrylpiperidine-1-carbonyl)-6-methyl-1-oxo-isoindolin-2-yl]piperidine-2,6-dione (2);
(3S)-3-[5-[(1R,5S)-3-benzhydryl-8-azabicyclo[3.2.1]octane-8-carbonyl]-1-oxo-isoindolin-2-yl]piperidine-2,6-dione (3);
(3S)-3-[5-(7-benzhydryl-3-oxa-9-azabicyclo[3.3.1]nonane-9-carbonyl)-1-oxo-isoindolin-2-yl]piperidine-2,6-dione (4);
(3S)-3-[5-[4-[(2-chlorophenyl)-phenyl-methyl]piperidine-1-carbonyl]-6-methyl-1-oxo-isoindolin-2-yl]piperidine-2,6-dione (5);
(3S)-3-[5-[(1R,4R)-5-benzhydryl-2-azabicyclo[2.2.1]heptane-2-carbonyl]-1-oxo-isoindolin-2-yl]piperidine-2,6-dione (6);
(3S)-3-[5-[(1R,5S)-3-benzhydryl-8-azabicyclo[3.2.1]octane-8-carbonyl]-6-fluoro-1-oxo-isoindolin-2-yl]piperidine-2,6-dione (7);
(3S)-3-[5-[(1R,5S)-3-benzhydryl-8-azabicyclo[3.2.1]octane-8-carbonyl]-4-methyl-1-oxo-isoindolin-2-yl]piperidine-2,6-dione (8);
(3S)-3-[5-[4-[(R)-(2-chlorophenyl)-phenyl-methyl]piperidine-1-carbonyl]-1-oxo-isoindolin-2-yl]piperidine-2,6-dione (9);
(3S)-3-[5-(4-benzhydrylpiperidine-1-carbonyl)-6-(difluoromethyl)-1-oxo-isoindolin-2-yl]piperidine-2,6-dione (10);
(3S)-3-[5-[4-[(R)-(2-chlorophenyl)-phenyl-methyl]piperidine-1-carbonyl]-6-methyl-1-oxo-isoindolin-2-yl]piperidine-2,6-dione (12);
(3R)-3-[5-[4-[(R)-(2-chlorophenyl)-phenyl-methyl]piperidine-1-carbonyl]-6-methyl-1-oxo-isoindolin-2-yl]piperidine-2,6-dione (17);
(3S)-3-[5-[4-[(R)-(2-chloro-3-methoxy-phenyl)-phenyl-methyl]piperidine-1-carbonyl]-6-methyl-1-oxo-isoindolin-2-yl]piperidine-2,6-dione (19);
(3S)-3-[5-[4-[(S)-(2-chloro-3-methoxy-phenyl)-phenyl-methyl]piperidine-1-carbonyl]-6-methyl-1-oxo-isoindolin-2-yl]piperidine-2,6-dione (20);
(3S)-3-[5-[4-[(2-chlorophenyl)-phenyl-methyl]piperidine-1-carbonyl]-6-[(dimethylamino)methyl]-1-oxo-isoindolin-2-yl]piperidine-2,6-dione (22);
[2-chloro-3-[[1-[2-[(3S)-2,6-dioxo-3-piperidyl]-6-methyl-1-oxo-isoindoline-5-carbonyl]-4-piperidyl]-phenyl-methyl]phenyl]boronic acid (23);
(3S)-3-[5-[4-[(S)-[2-chloro-3-[2-(dimethylamino) ethoxy]phenyl]-phenyl-methyl]piperidine-1-carbonyl]-6-methyl-1-oxo-isoindolin-2-yl]piperidine-2,6-dione hydrochloride (24);
(3S)-3-[5-[4-[(S)-[2-chloro-3-(2-hydroxyethoxy)phenyl]-phenyl-methyl]piperidine-1-carbonyl]-6-methyl-1-oxo-isoindolin-2-yl]piperidine-2,6-dione (25);
(3S)-3-[5-[4-[(R)-[2-chloro-3-[2-(dimethylamino) ethoxy]phenyl]-phenyl-methyl]piperidine-1-carbonyl]-6-methyl-1-oxo-isoindolin-2-yl]piperidine-2,6-dione hydrochloride (26);
(3S)-3-[5-[4-[[2-chloro-3-(methylamino)phenyl]-phenyl-methyl]piperidine-1-carbonyl]-6-methyl-1-oxo-isoindolin-2-yl]piperidine-2,6-dione (27);
(3S)-3-[5-[4-[[2-chloro-3-(dimethylamino)phenyl]-phenyl-methyl]piperidine-1-carbonyl]-6-methyl-1-oxo-isoindolin-2-yl]piperidine-2,6-dione (28);
(3S)-3-[5-[4-[(R)-(2-chloro-3-hydroxy-phenyl)-phenyl-methyl]piperidine-1-carbonyl]-6-methyl-1-oxo-isoindolin-2-yl]piperidine-2,6-dione (29);
(3S)-3-[5-[4-[(2-chloro-3-oxazol-2-yl-phenyl)-phenyl-methyl]piperidine-1-carbonyl]-6-methyl-1-oxo-isoindolin-2-yl]piperidine-2,6-dione (30);
(3S)-3-[5-[4-[(R)-[2-chloro-3-(2-hydroxyethoxy)phenyl]-phenyl-methyl]piperidine-1-carbonyl]-6-methyl-1-oxo-isoindolin-2-yl]piperidine-2,6-dione (31);
(3S)-3-[5-[4-[(2-chlorophenyl)-isothiazol-5-yl-methyl]piperidine-1-carbonyl]-6-methyl-1-oxo-isoindolin-2-yl]piperidine-2,6-dione (32);
(3S)-3-[5-[4-[(S)-[2-chloro-3-[(3R)-1-methylpyrrolidin-3-yl]oxy-phenyl]-phenyl-methyl]piperidine-1-carbonyl]-6-methyl-1-oxo-isoindolin-2-yl]piperidine-2,6-dione hydrochloride (33);
(3S)-3-[5-[4-[(R)-[2-chloro-3-[(3S)-1-methylpyrrolidin-3-yl]oxy-phenyl]-phenyl-methyl]piperidine-1-carbonyl]-6-methyl-1-oxo-isoindolin-2-yl]piperidine-2,6-dione hydrochloride (34);
(3S)-3-[5-[4-[(S)-[2-chloro-3-[(3S)-1-methylpyrrolidin-3-yl]oxy-phenyl]-phenyl-methyl]piperidine-1-carbonyl]-6-methyl-1-oxo-isoindolin-2-yl]piperidine-2,6-dione hydrochloride (35);
N-[2-chloro-3-[[1-[2-[(3S)-2,6-dioxo-3-piperidyl]-6-methyl-1-oxo-isoindoline-5-carbonyl]-4-piperidyl]-phenyl-methyl]phenyl]acetamide (36);
(3S)-3-[5-[4-[(S)-[2-chloro-3-[[(3R)-1-methylpyrrolidin-3-yl]methoxy]phenyl]-phenyl-methyl]piperidine-1-carbonyl]-6-methyl-1-oxo-isoindolin-2-yl]piperidine-2,6-dione hydrochloride (37);
(3S)-3-[5-[4-[(S)-[2-chloro-3-[3-(dimethylamino)propoxy]phenyl]-phenyl-methyl]piperidine-1-carbonyl]-6-methyl-1-oxo-isoindolin-2-yl]piperidine-2,6-dione hydrochloride (38);
(3S)-3-[5-[4-[[2-chloro-3-[2-(dimethylamino)ethyl]phenyl]-phenyl-methyl]piperidine-1-carbonyl]-6-methyl-1-oxo-isoindolin-2-yl]piperidine-2,6-dione hydrochloride (39);
(3S)-3-[5-[4-[(S)-[2-chloro-3-[(3-methylimidazol-4-yl)methoxy]phenyl]-phenyl-methyl]piperidine-1-carbonyl]-6-methyl-1-oxo-isoindolin-2-yl]piperidine-2,6-dione hydrochloride (40);
(3S)-3-[5-[4-[(R)-[2-chloro-3-[(3-methylimidazol-4-yl)methoxy]phenyl]-phenyl-methyl]piperidine-1-carbonyl]-6-methyl-1-oxo-isoindolin-2-yl]piperidine-2,6-dione (41);
(3S)-3-[5-[4-[(R)-[2-chloro-3-(1-methylazetidin-3-yl)oxy-phenyl]-phenyl-methyl]piperidine-1-carbonyl]-6-methyl-1-oxo-isoindolin-2-yl]piperidine-2,6-dione (42);
(3S)-3-[5-[4-[(S)-[2-chloro-3-[(1-methylimidazol-2-yl)methoxy]phenyl]-phenyl-methyl]piperidine-1-carbonyl]-6-methyl-1-oxo-isoindolin-2-yl]piperidine-2,6-dione hydrochloride (43);
3S)-3-[5-[4-[(S)-[2-chloro-3-[(1-methylimidazol-4-yl)methoxy]phenyl]-phenyl-methyl]piperidine-1-carbonyl]-6-methyl-1-oxo-isoindolin-2-yl]piperidine-2,6-dione hydrochloride (44);
(3S)-3-[5-[4-[(R)-[2-chloro-3-[2-(methylamino) ethoxy]phenyl]-phenyl-methyl]piperidine-1-carbonyl]-6-methyl-1-oxo-isoindolin-2-yl]piperidine-2,6-dione hydrochloride (45);
(3S)-3-[5-[4-[(R)-[3-(2-aminoethoxy)-2-chloro-phenyl]-phenyl-methyl]piperidine-1-carbonyl]-6-methyl-1-oxo-isoindolin-2-yl]piperidine-2,6-dione hydrochloride (46);
(3S)-3-[5-[4-[(R)-[2-chloro-3-[(1-methylimidazol-2-yl)methoxy]phenyl]-phenyl-methyl]piperidine-1-carbonyl]-6-methyl-1-oxo-isoindolin-2-yl]piperidine-2,6-dione hydrochloride (47);
(3S)-3-[5-[4-[(R)-[2-chloro-3-[(1-methylimidazol-4-yl)methoxy]phenyl]-phenyl-methyl]piperidine-1-carbonyl]-6-methyl-1-oxo-isoindolin-2-yl]piperidine-2,6-dione hydrochloride (48);
(3S)-3-[5-[4-[(R)-[2-chloro-3-[(2-methyl-2-azaspiro[3.3]heptan-6-yl)oxy]phenyl]-phenyl-methyl]piperidine-1-carbonyl]-6-methyl-1-oxo-isoindolin-2-yl]piperidine-2,6-dione (49);
(3S)-3-[5-[4-[(R)-[2-chloro-3-[(3S)-1-(2,2,2-trifluoroethyl) pyrrolidin-3-yl]oxy-phenyl]-phenyl-methyl]piperidine-1-carbonyl]-6-methyl-1-oxo-isoindolin-2-yl]piperidine-2,6-dione (50);
3S)-3-[5-[4-[(R)-(2-chloro-3-isopropoxy-phenyl)-phenyl-methyl]piperidine-1-carbonyl]-6-methyl-1-oxo-isoindolin-2-yl]piperidine-2,6-dione (51);
(3S)-3-[5-[4-[(R)-[2-chloro-3-[(3R)-1-(2,2,2-trifluoroethyl) pyrrolidin-3-yl]oxy-phenyl]-phenyl-methyl]piperidine-1-carbonyl]-6-methyl-1-oxo-isoindolin-2-yl]piperidine-2,6-dione (52);
(3S)-3-[5-[4-[(S)-[2-chloro-3-(6,8-dihydro-5H-imidazo[1,5-a]pyrazin-7-yl)phenyl]-phenyl-methyl]piperidine-1-carbonyl]-6-methyl-1-oxo-isoindolin-2-yl]piperidine-2,6-dione (53);
(3S)-3-[5-[4-[(R)-[2-chloro-3-[(3R)-1-(2,2-difluoroethyl) pyrrolidin-3-yl]oxy-phenyl]-phenyl-methyl]piperidine-1-carbonyl]-6-methyl-1-oxo-isoindolin-2-yl]piperidine-2,6-dione (54);
(3S)-3-[5-[4-[(R)-[2-chloro-3-(cyclobutoxy)phenyl]-phenyl-methyl]piperidine-1-carbonyl]-6-methyl-1-oxo-isoindolin-2-yl]piperidine-2,6-dione (55);
N-[2-chloro-3-[[1-[2-[(3S)-2,6-dioxo-3-piperidyl]-6-methyl-1-oxo-isoindoline-5-carbonyl]-4-piperidyl]-phenyl-methyl]phenyl]-2-hydroxy-acetamide (56);
N-[2-[2-chloro-3-[(R)-[1-[2-[(3S)-2,6-dioxo-3-piperidyl]-6-methyl-1-oxo-isoindoline-5-carbonyl]-4-piperidyl]-phenyl-methyl]phenoxy]ethyl]acetamide (57);
(3S)-3-[5-[4-[(R)-[3-[(1S,3S)-3-aminocyclopentoxy]-2-chloro-phenyl]-phenyl-methyl]piperidine-1-carbonyl]-6-methyl-1-oxo-isoindolin-2-yl]piperidine-2,6-dione hydrochloride (58);
(3S)-3-[5-[4-[(R)-[3-[(2S)-2-amino-3,3-difluoro-propoxy]-2-chloro-phenyl]-phenyl-methyl]piperidine-1-carbonyl]-6-methyl-1-oxo-isoindolin-2-yl]piperidine-2,6-dione hydrochloride (59);
(3S)-3-[5-[4-[(R)-[2-chloro-3-(2-methoxyethoxy)phenyl]-phenyl-methyl]piperidine-1-carbonyl]-6-methyl-1-oxo-isoindolin-2-yl]piperidine-2,6-dione (60);
(3S)-3-[5-[4-[(R)-[3-(3-aminopropoxy)-2-chloro-phenyl]-phenyl-methyl]piperidine-1-carbonyl]-6-methyl-1-oxo-isoindolin-2-yl]piperidine-2,6-dione hydrochloride (61);
(3S)-3-[5-[4-[(R)-[3-[(2S)-2-amino-3-methyl-butoxy]-2-chloro-phenyl]-phenyl-methyl]piperidine-1-carbonyl]-6-methyl-1-oxo-isoindolin-2-yl]piperidine-2,6-dione hydrochloride (62);
(3S)-3-[5-[4-[indolin-4-yl(phenyl)methyl]piperidine-1-carbonyl]-6-methyl-1-oxo-isoindolin-2-yl]piperidine-2,6-dione hydrochloride (63);
(3S)-3-[5-[4-[(R)-[2-chloro-3-[2-[(3S)-pyrrolidin-3-yl]ethoxy]phenyl]-phenyl-methyl]piperidine-1-carbonyl]-6-methyl-1-oxo-isoindolin-2-yl]piperidine-2,6-dione 4-methylbenzenesulfonic acid (64);
(3S)-3-[5-[4-[(R)-[2-chloro-3-(methylamino)phenyl]-phenyl-methyl]piperidine-1-carbonyl]-6-methyl-1-oxo-isoindolin-2-yl]piperidine-2,6-dione (65);
(3S)-3-[5-[4-[(R)-[3-[(2S)-2-aminopropoxy]-2-chloro-phenyl]-phenyl-methyl]piperidine-1-carbonyl]-6-methyl-1-oxo-isoindolin-2-yl]piperidine-2,6-dione hydrochloride (66);
(3S)-3-[5-[4-[(R)-[3-[(2S)-3-amino-2-fluoro-propoxy]-2-chloro-phenyl]-phenyl-methyl]piperidine-1-carbonyl]-6-methyl-1-oxo-isoindolin-2-yl]piperidine-2,6-dione hydrochloride (67);
(3S)-3-[5-[4-[(R)-[3-[(3R)-3-aminopyrrolidin-1-yl]-2-chloro-phenyl]-phenyl-methyl]piperidine-1-carbonyl]-6-methyl-1-oxo-isoindolin-2-yl]piperidine-2,6-dione hydrochloride (68);
(3S)-3-[5-[4-[(R)-[3-[(1-aminocyclopropyl)methoxy]-2-chloro-phenyl]-phenyl-methyl]piperidine-1-carbonyl]-6-methyl-1-oxo-isoindolin-2-yl]piperidine-2,6-dione hydrochloride (69);
(3S)-3-[5-[4-[(R)-[2-chloro-3-[(3R)-3-hydroxypyrrolidin-1-yl]phenyl]-phenyl-methyl]piperidine-1-carbonyl]-6-methyl-1-oxo-isoindolin-2-yl]piperidine-2,6-dione (70);
(3S)-3-[5-[4-[(R)-[3-[(2R)-3-amino-2-fluoro-propoxy]-2-chloro-phenyl]-phenyl-methyl]piperidine-1-carbonyl]-6-methyl-1-oxo-isoindolin-2-yl]piperidine-2,6-dione hydrochloride (71);
(3S)-3-[5-[4-[(R)-[2-chloro-3-[(3R)-1-(2-fluoroethyl) pyrrolidin-3-yl]oxy-phenyl]-phenyl-methyl]piperidine-1-carbonyl]-6-methyl-1-oxo-isoindolin-2-yl]piperidine-2,6-dione hydrochloride (72);
(3S)-3-[5-[4-[(R)-[2-chloro-3-(1H-imidazol-5-ylmethoxy)phenyl]-phenyl-methyl]piperidine-1-carbonyl]-6-methyl-1-oxo-isoindolin-2-yl]piperidine-2,6-dione hydrochloride (73);
(3S)-3-[5-[4-[(R)-[2-chloro-3-(oxazol-5-ylmethoxy)phenyl]-phenyl-methyl]piperidine-1-carbonyl]-6-methyl-1-oxo-isoindolin-2-yl]piperidine-2,6-dione (74);
(3S)-3-[5-[4-[(R)-[2-chloro-3-(oxazol-4-ylmethoxy)phenyl]-phenyl-methyl]piperidine-1-carbonyl]-6-methyl-1-oxo-isoindolin-2-yl]piperidine-2,6-dione (75);
(3S)-3-[5-[4-[rel-(S)-indolin-4-yl(phenyl)methyl]piperidine-1-carbonyl]-6-methyl-1-oxo-isoindolin-2-yl]piperidine-2,6-dione (76);
(3S)-3-[5-[4-[rel-(R)-indolin-4-yl(phenyl)methyl]piperidine-1-carbonyl]-6-methyl-1-oxo-isoindolin-2-yl]piperidine-2,6-dione (77);
(3S)-3-[5-[4-[(R)-[3-[(2S)-2-amino-3,3,3-trifluoro-propoxy]-2-chloro-phenyl]-phenyl-methyl]piperidine-1-carbonyl]-6-methyl-1-oxo-isoindolin-2-yl]piperidine-2,6-dione (78);
(3S)-3-[5-[4-[(R)-[3-[((1r,3r)-3-aminocyclobutyl)amino]-2-chloro-phenyl]-phenyl-methyl]piperidine-1-carbonyl]-6-methyl-1-oxo-isoindolin-2-yl]piperidine-2,6-dione hydrochloride (79);
(3S)-3-[5-[4-[(R)-[3-[(1S)-2-amino-1-methyl-ethoxy]-2-chloro-phenyl]-phenyl-methyl]piperidine-1-carbonyl]-6-methyl-1-oxo-isoindolin-2-yl]piperidine-2,6-dione hydrochloride (80);
(3S)-3-[5-[4-[(R)-[3-[(2R)-2-amino-3,3-difluoro-propoxy]-2-chloro-phenyl]-phenyl-methyl]piperidine-1-carbonyl]-6-methyl-1-oxo-isoindolin-2-yl]piperidine-2,6-dione hydrochloride (81);
(3S)-3-[5-[4-[rel-(S)-[3-(2-aminoethoxy)-2-fluoro-phenyl]-phenyl-methyl]piperidine-1-carbonyl]-6-methyl-1-oxo-isoindolin-2-yl]piperidine-2,6-dione hydrochloride (82);
(3S)-3-[5-[4-[(R)-[3-(3-amino-2,2-difluoro-propoxy)-2-chloro-phenyl]-phenyl-methyl]piperidine-1-carbonyl]-6-methyl-1-oxo-isoindolin-2-yl]piperidine-2,6-dione hydrochloride (83);
(3S)-3-[5-[4-[rel-(R)-(2-chlorophenyl)-(3-pyridyl)methyl]piperidine-1-carbonyl]-6-methyl-1-oxo-isoindolin-2-yl]piperidine-2,6-dione hydrochloride (84);
(3S)-3-[5-[4-[(R)-[3-(2-amino-4,4-difluoro-butoxy)-2-chloro-phenyl]-phenyl-methyl]piperidine-1-carbonyl]-6-methyl-1-oxo-isoindolin-2-yl]piperidine-2,6-dione hydrochloride (85);
(3S)-3-[5-[4-[(R)-[3-[(2S)-2-aminobutoxy]-2-chloro-phenyl]-phenyl-methyl]piperidine-1-carbonyl]-6-methyl-1-oxo-isoindolin-2-yl]piperidine-2,6-dione hydrochloride (86);
(3S)-3-[5-[4-[(R)-[3-[(2R)-2-amino-3,3,3-trifluoro-propoxy]-2-chloro-phenyl]-phenyl-methyl]piperidine-1-carbonyl]-6-methyl-1-oxo-isoindolin-2-yl]piperidine-2,6-dione (87);
(3S)-3-[5-[4-[(R)-[2-chloro-3-(isoxazol-4-ylmethoxy)phenyl]-phenyl-methyl]piperidine-1-carbonyl]-6-methyl-1-oxo-isoindolin-2-yl]piperidine-2,6-dione (88);
(3S)-3-[5-[4-[(R)-[2-chloro-3-(isoxazol-5-ylmethoxy)phenyl]-phenyl-methyl]piperidine-1-carbonyl]-6-methyl-1-oxo-isoindolin-2-yl]piperidine-2,6-dione (89);
(3S)-3-[5-[4-[(R)-(2-chloro-3-morpholino-phenyl)-phenyl-methyl]piperidine-1-carbonyl]-6-methyl-1-oxo-isoindolin-2-yl]piperidine-2,6-dione (90);
(3S)-3-[5-[4-[(R)-[2-chloro-3-(isoxazol-3-ylmethoxy)phenyl]-phenyl-methyl]piperidine-1-carbonyl]-6-methyl-1-oxo-isoindolin-2-yl]piperidine-2,6-dione (91);
(3S)-3-[5-[4-[(R)-[2-chloro-3-(oxazol-2-ylmethoxy)phenyl]-phenyl-methyl]piperidine-1-carbonyl]-6-methyl-1-oxo-isoindolin-2-yl]piperidine-2,6-dione (92);
(3S)-3-[6-methyl-1-oxo-5-[4-[phenyl(1,2,3,4-tetrahydroquinolin-5-yl)methyl]piperidine-1-carbonyl]isoindolin-2-yl]piperidine-2,6-dione (93);
(3S)-3-[5-[4-[(R)-[2-chloro-3-(2-hydroxyethylamino)phenyl]-phenyl-methyl]piperidine-1-carbonyl]-6-methyl-1-oxo-isoindolin-2-yl]piperidine-2,6-dione (94);
(3S)-3-[5-[4-[(R)-[3-[(1R)-2-amino-1-methyl-ethoxy]-2-chloro-phenyl]-phenyl-methyl]piperidine-1-carbonyl]-6-methyl-1-oxo-isoindolin-2-yl]piperidine-2,6-dione hydrochloride (95);
(3S)-3-[5-[4-[3,4-dihydro-2H-1,4-benzoxazin-8-yl(phenyl)methyl]piperidine-1-carbonyl]-6-methyl-1-oxo-isoindolin-2-yl]piperidine-2,6-dione (96);
(3S)-3-[5-[4-[indolin-6-yl(phenyl)methyl]piperidine-1-carbonyl]-6-methyl-1-oxo-isoindolin-2-yl]piperidine-2,6-dione (97);
(3S)-3-[5-[4-[(R)-(2-chloro-3-ethoxy-phenyl)-phenyl-methyl]piperidine-1-carbonyl]-6-methyl-1-oxo-isoindolin-2-yl]piperidine-2,6-dione (98);
(3S)-3-[5-[4-[(R)-[3-(2-aminoethylamino)-2-chloro-phenyl]-phenyl-methyl]piperidine-1-carbonyl]-6-methyl-1-oxo-isoindolin-2-yl]piperidine-2,6-dione hydrochloride (99);
(3S)-3-[5-[4-[(R)-[2-chloro-3-(trideuteriomethoxy)phenyl]-phenyl-methyl]piperidine-1-carbonyl]-6-methyl-1-oxo-isoindolin-2-yl]piperidine-2,6-dione (100);
(3S)-3-[5-[4-[(R)-[2-chloro-3-(2-fluoroethoxy)phenyl]-phenyl-methyl]piperidine-1-carbonyl]-6-methyl-1-oxo-isoindolin-2-yl]piperidine-2,6-dione (101);
(3S)-3-[5-[4-[(R)-[2-chloro-3-(cyclopropoxy)phenyl]-phenyl-methyl]piperidine-1-carbonyl]-6-methyl-1-oxo-isoindolin-2-yl]piperidine-2,6-dione (102);
3-[5-[4-[(R)-(2-chlorophenyl)-phenyl-methyl]piperidine-1-carbonyl]-6-methyl-1-oxo-isoindolin-2-yl]piperidine-2,6-dione (103);
(3S)-3-[6-methyl-1-oxo-5-[4-[rel-(S)-phenyl(1,2,3,4-tetrahydroquinolin-5-yl)methyl]piperidine-1-carbonyl]isoindolin-2-yl]piperidine-2,6-dione (104);
(3S)-3-[5-[4-[(2-chloro-3-pyridyl)-phenyl-methyl]piperidine-1-carbonyl]-6-methyl-1-oxo-isoindolin-2-yl]piperidine-2,6-dione (105);
(3S)-3-[6-methyl-1-oxo-5-[4-[phenyl(2,3,4,5-tetrahydro-1H-1-benzazepin-6-yl)methyl]piperidine-1-carbonyl]isoindolin-2-yl]piperidine-2,6-dione (106);
(3S)-3-[6-methyl-1-oxo-5-[4-[phenyl(1,2,3,5-tetrahydro-4,1-benzoxazepin-6-yl)methyl]piperidine-1-carbonyl]isoindolin-2-yl]piperidine-2,6-dione (107);
(3S)-3-[5-[4-[2,3-dihydrobenzofuran-4-yl(phenyl)methyl]piperidine-1-carbonyl]-6-methyl-1-oxo-isoindolin-2-yl]piperidine-2,6-dione (108);
(3S)-3-[5-[4-[1H-indazol-4-yl(phenyl)methyl]piperidine-1-carbonyl]-6-methyl-1-oxo-isoindolin-2-yl]piperidine-2,6-dione (109);
(3S)-3-[5-[4-[(2-chloro-3-methoxy-phenyl)-deuterio-phenyl-methyl]piperidine-1-carbonyl]-6-methyl-1-oxo-isoindolin-2-yl]piperidine-2,6-dione (110);
(3S)-3-[5-[4-[[2-chloro-3-(trifluoromethoxy)phenyl]-phenyl-methyl]piperidine-1-carbonyl]-6-methyl-1-oxo-isoindolin-2-yl]piperidine-2,6-dione (111);
(3S)-3-[rel-(1S)-6-[4-[(R)-(2-chlorophenyl)-phenyl-methyl]piperidine-1-carbonyl]-1,5-dimethyl-3-oxo-isoindolin-2-yl]piperidine-2,6-dione (112);
(3S)-3-[5-[4-[rel-(S)-(2-chloro-4-fluoro-3-methoxy-phenyl)-phenyl-methyl]piperidine-1-carbonyl]-6-methyl-1-oxo-isoindolin-2-yl]piperidine-2,6-dione (113); or
(3S)-3-[5-[4-[[3-(2-aminoethoxy)-2-bromo-phenyl]-phenyl-methyl]piperidine-1-carbonyl]-6-methyl-1-oxo-isoindolin-2-yl]piperidine-2,6-dione hydrochloride (114).

One embodiment provides a compound of Formula (I), or stereoisomers, tautomers, or salts thereof, wherein said compound is:
(3S)-3-[3-[4-[(2-chlorophenyl)-phenyl-methyl]piperidine-1-carbonyl]-7-oxo-5H-pyrrolo[3,4-b]pyridin-6-yl]piperidine-2,6-dione (11);
(3S)-3-[3-[4-[(R)-(2-chloro-3-methoxy-phenyl)-phenyl-methyl]piperidine-1-carbonyl]-7-oxo-5H-pyrrolo[3,4-b]pyridin-6-yl]piperidine-2,6-dione (13);
(3S)-3-[3-[4-[(S)-(2-chloro-3-methoxy-phenyl)-phenyl-methyl]piperidine-1-carbonyl]-7-oxo-5H-pyrrolo[3,4-b]pyridin-6-yl]piperidine-2,6-dione (14);
(3S)-3-[3-[4-[(R)-(2-chlorophenyl)-phenyl-methyl]piperidine-1-carbonyl]-7-oxo-5H-pyrrolo[3,4-b]pyridin-6-yl]piperidine-2,6-dione (15);
(3S)-3-[3-[4-[(S)-(2-chlorophenyl)-phenyl-methyl]piperidine-1-carbonyl]-7-oxo-5H-pyrrolo[3,4-b]pyridin-6-yl]piperidine-2,6-dione (16); or (3S)-3-[3-[4-[(2-chlorophenyl)-phenyl-methyl]piperidine-1-carbonyl]-2-methyl-7-oxo-5H-pyrrolo[3,4-b]pyridin-6-yl]piperidine-2,6-dione (18).

One embodiment provides a compound of Formula (I), or stereoisomers, tautomers, or salts thereof, wherein said compound is (3S)-3-[3-[4-[(2-chlorophenyl)-phenyl-methyl]piperidine-1-carbonyl]-2-methyl-7-oxo-5H-pyrrolo[3,4-b]pyrazin-6-yl]piperidine-2,6-dione (21).

The compounds of Formula (I) or stereoisomers, tautomers, or salts thereof, are useful to decrease the level of CDK2 protein.

The present invention may be embodied in other specific forms without departing from the spirit or essential attributes thereof. This invention encompasses all combinations of the aspects and/or embodiments of the invention noted herein. It is understood that any and all embodiments of the present invention may be taken in conjunction with any other embodiment or embodiments to describe additional embodiments. It is also to be understood that each individual element of the embodiments is meant to be combined with any and all other elements from any embodiment to describe an additional embodiment.

The features and advantages of the invention may be more readily understood by those of ordinary skill in the art upon reading the following detailed description. It is to be appreciated that certain features of the invention that are, for clarity reasons, described above and below in the context of separate embodiments, may also be combined to form a single embodiment. Conversely, various features of the invention that are, for brevity reasons, described in the context of a single embodiment, may also be combined so as to form sub-combinations thereof. Embodiments identified herein as exemplary or preferred are intended to be illustrative and not limiting.

Unless specifically stated otherwise herein, references made in the singular may also include the plural. For example, “a” and “an” may refer to either one, or one or more.

As used herein, the phrase “compounds and/or salts thereof” refers to at least one compound, at least one salt of the compounds, or a combination thereof. For example, compounds of Formula (I) and/or salts thereof includes a compound of Formula (I); two compounds of Formula (I); a salt of a compound of Formula (I); a compound of Formula (I) and one or more salts of the compound of Formula (I); and two or more salts of a compound of Formula (I).

Unless otherwise indicated, any atom with unsatisfied valences is assumed to have hydrogen atoms sufficient to satisfy the valences.

The definitions set forth herein take precedence over definitions set forth in any patent, patent application, and/or patent application publication incorporated herein by reference.

Listed below are definitions of various terms used to describe the present invention. These definitions apply to the terms as they are used throughout the specification (unless they are otherwise limited in specific instances) either individually or as part of a larger group.

Throughout the specification, groups and substituents thereof may be chosen by one skilled in the field to provide stable moieties and compounds.

In accordance with a convention used in the art,

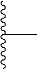

is used in structural formulas herein to depict the bond that is the point of attachment of the moiety or substituent to the core or backbone structure.

The term “amino” refers to the group-NH₂.

The term “oxo” refers to the group ═O.

The compounds of the present invention include all isotopes of atoms occurring in the present compounds. Isotopes include those atoms having the same atomic number but different mass numbers. By way of general example and without limitation, isotopes of hydrogen include deuterium (D) and tritium (T). Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed.

As used herein, the term “tautomer” refers to each of two or more isomers of a compound that exist together in equilibrium, and are readily interchanged by migration of an atom or group within the molecule. For example, one skilled in the art would readily understand that a 1,2,3-triazole exists in two tautomeric forms as defined above:

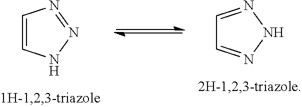 [SMILES: c1c[nH]nn1 ; c1cn[nH]n1]

Thus, this disclosure is intended to cover all possible tautomers even when a structure depicts only one of them. For example, the compounds of Formula (I) can exist in tautomer forms:

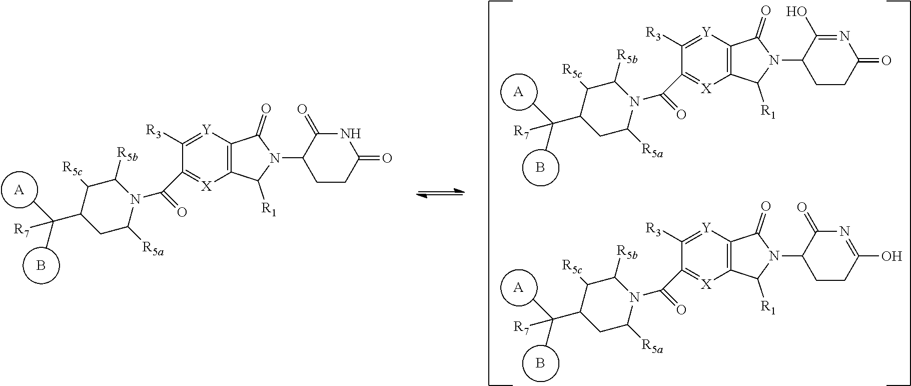

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The compounds of Formula (I) can form salts which are also within the scope of this invention. Unless otherwise indicated, reference to an inventive compound is understood to include reference to one or more salts thereof. The term “salt(s)” denotes acidic salt(s) formed with inorganic and/or organic acids. In addition, the term “salt(s) may include zwitterions (inner salts), e.g., when a compound of Formula (I) contains both a basic moiety, such as an amine or a pyridine or imidazole ring, and an acidic moiety, such as a carboxylic acid. Pharmaceutically acceptable (i.e., non-toxic, physiologically acceptable) salts are preferred, such as, for example, acceptable metal and amine salts in which the cation does not contribute significantly to the toxicity or biological activity of the salt. However, other salts may be useful, e.g., in isolation or purification steps which may be employed during preparation, and thus, are contemplated within the scope of the invention. Salts of the compounds of the formula (I) may be formed, for example, by reacting a compound of the Formula (I) with an amount of acid or base, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, maleates (formed with maleic acid), 2-hydroxyethanesulfonates, lactates, methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

The compounds of Formula (I) can be provided as amorphous solids or crystalline solids. Lyophilization can be employed to provide the compounds of Formula (I) as a solid.

It should further be understood that solvates (e.g., hydrates) of the compounds of Formula (I) are also within the scope of the present invention. The term “solvate” means a physical association of a compound of Formula (I) with one or more solvent molecules, whether organic or inorganic. This physical association includes hydrogen bonding. In certain instances, the solvate will be capable of isolation, for example when one or more solvent molecules are incorporated in the crystal lattice of the crystalline solid. “Solvate” encompasses both solution-phase and isolable solvates. Exemplary solvates include hydrates, ethanolates, methanolates, isopropanolates, acetonitrile solvates, and ethyl acetate solvates. Methods of solvation are known in the art.

Various forms of prodrugs are well known in the art and are described in Rautio, J. et al., Nature Review Drug Discovery, 17, 559-587 (2018).

In addition, compounds of Formula (I), subsequent to their preparation, can be isolated and purified to obtain a composition containing an amount by weight equal to or greater than 99% of a compound of Formula (I) (“substantially pure”), which is then used or formulated as described herein. Such “substantially pure” compounds of Formula (I) are also contemplated herein as part of the present invention.

“Stable compound” and “stable structure” are meant to indicate a compound that is sufficiently robust to survive isolation to a useful degree of purity from a reaction mixture, and formulation into an efficacious therapeutic agent. The present invention is intended to embody stable compounds.

The term “CDK2” refers to cyclin-dependent kinase 2.

The terms “Cyclin-dependent kinase 2 degrader” and “CDK2 degrader” refers to an agent capable of reducing the level of the cyclin-dependent kinase 2 protein by degradation and/or inactivation and/or inhibition and/or reducing the expression levels of the cyclin-dependent kinase 2 protein, or a combination thereof.

As used herein, “cyclin-dependent kinase 2” protein is encoded by the CDK2 gene, (Gene ID: 1017 also known as CDKN2, p33 (CDK2). “Cyclin-dependent kinase 2” protein includes all human isoforms encoded by CDK2 gene as listed below:

(SEQ ID NO: 1)
Isoform 1 (P24941-1)
MENFQKVEKIGEGTYGVVYKARNKLTGEVVALKKIRLDTETEGVPSTA
IREISLLKELNHPNIVKLLDVIHTENKLYLVFEFLHQDLKKFMDASAL
TGIPLPLIKSYLFQLLQGLAFCHSHRVLHRDLKPQNLLINTEGAIKLA
DFGLARAFGVPVRTYTHEVVTLWYRAPEILLGCKYYSTAVDIWSLGCI
FAEMVTRRALFPGDSEIDQLFRIFRTLGTPDEVVWPGVTSMPDYKPSF
PKWARQDFSKVVPPLDEDGRSLLSQMLHYDPNKRISAKAALAHPFFQD
VTKPVPHLRL
(SEQ ID NO: 2)
Isoform 2 (P24941-2, CDK2deltaT)
MENFQKVEKIGEGTYGVVYKARNKLTGEVVALKKIRLDTETEGVPSTA
IREISLLKELNHPNIVKLLDVIHTENKLYLVFEFLHQDLKKFMDASAL
TGIPLPLIKSYLFQLLQGLAFCHSHRVLHRDLKPQNLLINTEGAIKLA
DFGLARAFGVPVRTYTHEVTRRALFPGDSEIDQLFRIFRTLGTPDEVV
WPGVTSMPDYKPSFPKWARQDFSKVVPPLDEDGRSLLSQMLHYDPNKR
ISAKAALAHPFFQDVTKPVPHLRL

As used herein, the term “contacting” refers to the bringing together of indicated moieties in an in vitro system or an in vivo system. For example, “contacting” CDK2 protein with a compound of Formula (I) includes the administration of a compound of the present invention to an individual or patient, such as a human, having CDK2 protein as, for example, introducing a compound of Formula (I) into a sample containing a cellular or purified preparation containing CDK2 protein.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease. In contrast, “prophylaxis” or “prevention” refers to administration to a subject who does not have a disease to prevent the disease from occurring. “Treat,” “treating,” and “treatment” does not encompass prophylaxis or prevention.

“Therapeutically effective amount” is intended to include an amount of a compound of the present invention alone or an amount of the combination of compounds claimed or an amount of a compound of the present invention in combination with other active ingredients effective to decrease the level of the CDK2 protein in the cells, or effective to treat proliferative disorders, such as cancer.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

The term “patient” includes human subjects.

The phrase “pharmaceutically acceptable carrier” as used herein means a pharmaceutically acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation, including, i.e., adjuvant, excipient or vehicle, such as diluents, preserving agents, fillers, flow regulating agents, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms; and not injurious to the patient.

The term “pharmaceutical composition” means a composition comprising a compound of the invention in combination with at least one additional pharmaceutically acceptable carrier.

### UTILITY

The compounds of Formula (I) are useful for the treatment of cancer.

In one embodiment, a method is provided for the treatment of cancer in a patient comprising administering to said patient a therapeutically effective amount of a compound according to Formula (I), or a stereoisomer, a tautomer, or a pharmaceutically acceptable salt thereof.

One aspect provides a method of treating a disease or disorder by decreasing the level of the CDK2 protein, the method comprising administering to a patient a therapeutically effective amount of an agent to decrease the CDK2 protein level. Suitable agents include small molecules and heterobifunctional molecules such as proteolysis targeting chimeras (PROTAC) molecule. In one embodiment, the disease or disorder is cancer. In another embodiment, the agent is a small compound having a molecular weight of 1000 or less. In a further embodiment, the agent is a compound of Formula (I), a stereoisomer, a tautomer, or a pharmaceutically acceptable salt thereof. In an additional embodiment, the agent is a proteolysis targeting chimera molecule. Examples of small molecules include molecular glues (Sasso et al., Biochemistry 2023, 62, 601-623).

In one embodiment, a method is provided for the treatment of a disease or disorder in a patient comprising administering to said patient a therapeutically effective amount of an agent to decrease the CDK2 protein level, wherein: the CDK2 protein is the amino acid sequence encoded by SEQ ID NOs: 1 or 2. In one embodiment, the disease or disorder is cancer. In another embodiment, the agent is a small compound having a molecular weight of 1000 or less. In a further embodiment, the agent is a compound of Formula (I), a stereoisomer, a tautomer, or a pharmaceutically acceptable salt thereof. In an additional embodiment, the agent is a proteolysis targeting chimera molecule.

In Embodiment 1, a method is provided for the treatment of disease or disorder in a patient comprising administering to said patient a therapeutically effective amount of an agent to decrease the CDK2 protein level, wherein the CDK2 protein level is decreased by at least 70%. Included in this embodiment is a method where the disease or disorder is cancer. Also included in this embodiment is a method wherein the agent is the compound of Formula (I), a stereoisomer, a tautomer, or a pharmaceutically acceptable salt thereof.

In Embodiment 2, a method is provided for the treatment of disease or disorder in a patient comprising administering to said patient a therapeutically effective amount of an agent to decrease the CDK2 protein level, wherein the CDK2 protein level is decreased by at least 75%. Included in this embodiment is a method where the disease or disorder is cancer. Also included in this embodiment is a method wherein the agent is the compound of Formula (I), a stereoisomer, a tautomer, or a pharmaceutically acceptable salt thereof.

In Embodiment 3, a method is provided for the treatment of disease or disorder in a patient comprising administering to said patient a therapeutically effective amount of an agent to decrease the CDK2 protein level, wherein the CDK2 protein level is decreased by at least 80%. Included in this embodiment is a method where the disease or disorder is cancer. Also included in this embodiment is a method wherein the agent is the compound of Formula (I), a stereoisomer, a tautomer, or a pharmaceutically acceptable salt thereof.

In Embodiment 4, a method is provided for the treatment of disease or disorder in a patient comprising administering to said patient a therapeutically effective amount of an agent to decrease the CDK2 protein level, wherein the CDK2 protein level is decreased by at least 85%. Included in this embodiment is a method where the disease or disorder is cancer. Also included in this embodiment is a method wherein the agent is the compound of Formula (I), a stereoisomer, a tautomer, or a pharmaceutically acceptable salt thereof.

In Embodiment 5, a method is provided for the treatment of disease or disorder in a patient comprising administering to said patient a therapeutically effective amount of an agent to decrease the CDK2 protein level, wherein the CDK2 protein level is decreased by at least 90%. Included in this embodiment is a method where the disease or disorder is cancer. Also included in this embodiment is a method wherein the agent is the compound of Formula (I), a stereoisomer, a tautomer, or a pharmaceutically acceptable salt thereof.

In Embodiment 6, a method is provided for the treatment of disease or disorder in a patient comprising administering to said patient a therapeutically effective amount of an agent to decrease the CDK2 protein level, wherein the CDK2 protein level is decreased by at least 92%. Included in this embodiment is a method where the disease or disorder is cancer. Also included in this embodiment is a method wherein the agent is the compound of Formula (I), a stereoisomer, a tautomer, or a pharmaceutically acceptable salt thereof.

In Embodiment 7, a method is provided for the treatment of disease or disorder in a patient comprising administering to said patient a therapeutically effective amount of an agent to decrease the CDK2 protein level, wherein the CDK2 protein level is decreased by at least 94%. Included in this embodiment is a method where the disease or disorder is cancer. Also included in this embodiment is a method wherein the agent is the compound of Formula (I), a stereoisomer, a tautomer, or a pharmaceutically acceptable salt thereof.

In Embodiment 8, a method is provided for the treatment of disease or disorder in a patient comprising administering to said patient a therapeutically effective amount of an agent to decrease the CDK2 protein level, wherein the CDK2 protein level is decreased by at least 95%. Included in this embodiment is a method where the disease or disorder is cancer. Also included in this embodiment is a method wherein the agent is the compound of Formula (I), a stereoisomer, a tautomer, or a pharmaceutically acceptable salt thereof.

In Embodiment 9, a method is provided for the treatment of disease or disorder in a patient comprising administering to said patient a therapeutically effective amount of an agent to decrease the CDK2 protein level, wherein the CDK2 protein level is decreased by at least 96%. Included in this embodiment is a method where the disease or disorder is cancer. Also included in this embodiment is a method wherein the agent is the compound of Formula (I), a stereoisomer, a tautomer, or a pharmaceutically acceptable salt thereof. In Embodiment 10, a method is provided for the treatment of disease or disorder in a patient comprising administering to said patient a therapeutically effective amount of an agent to decrease the CDK2 protein level, wherein the CDK2 protein level is decreased by at least 98%. Included in this embodiment is a method where the disease or disorder is cancer. Also included in this embodiment is a method wherein the agent is the compound of Formula (I), a stereoisomer, a tautomer, or a pharmaceutically acceptable salt thereof.

In Embodiments 1 to 10, the decrease in the protein levels of the CDK2 protein can be measured using the CDK2 Cellular Degradation assay described hereinbelow.

Types of cancers that may be treated with the compound of Formula (I) include, but are not limited to, brain cancers, skin cancers, bladder cancers, ovarian cancers, breast cancers, gastric cancers, pancreatic cancers, prostate cancers, colon cancers, blood cancers, lung cancers and bone cancers. Examples of such cancer types include neuroblastoma, intestine carcinoma such as rectum carcinoma, colon carcinoma, anal cancer, familiar adenomatous polyposis carcinoma and hereditary non-polyposis colorectal cancer, esophageal carcinoma, nasopharyngeal carcinoma, labial carcinoma, larynx carcinoma, hypopharynx carcinoma, tongue carcinoma, salivary gland carcinoma, thymic carcinoma, esophagogastric cancer, gastric carcinoma, adenocarcinoma, medullary thyroid carcinoma, papillary thyroid carcinoma, renal carcinoma, kidney parenchymal carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors such as glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia lymphoma, diffuse large B-cell lymphoma (DLBCL), hepatocellular carcinoma, gall bladder carcinoma, bronchial carcinoma, small cell lung carcinoma, non-small cell lung carcinoma, mesothelioma, multiple myeloma, basalioma, teratoma, retinoblastoma, choroid melanoma, seminoma, rhabdomyosarcoma, craniopharyngioma, osteosarcoma, chondrosarcoma, myosarcoma, liposarcoma, fibrosarcoma, Ewing sarcoma and plasmocytoma.

In one embodiment, a method is provided for the treatment of cancer, in a patient comprising administering to said patient a therapeutically effective amount of a compound according to Formula (I), or a stereoisomer, a tautomer, or a pharmaceutically acceptable salt thereof, wherein said cancer has amplification, overexpression, or detectable expression of the CCNE1 gene.

In one embodiment, a method is provided for the treatment of cancer, in a patient comprising administering to said patient a therapeutically effective amount of a compound according to Formula (I), or a stereoisomer, a tautomer, or a pharmaceutically acceptable salt thereof, wherein said cancer is melanoma.

In one embodiment, a method is provided for the treatment of cancer, in a patient comprising administering to said patient a therapeutically effective amount of a compound according to Formula (I), or a stereoisomer, a tautomer, or a pharmaceutically acceptable salt thereof, wherein said cancer is lung cancer, including small cell lung cancer (SCLC) and non-small cell lung cancer (NSCLC).

In one embodiment, a method is provided for the treatment of cancer, in a patient comprising administering to said patient a therapeutically effective amount of a compound according to Formula (I), or a stereoisomer, a tautomer, or a pharmaceutically acceptable salt thereof, wherein said cancer is mesothelioma.

In one embodiment, a method is provided for the treatment of cancer, in a patient comprising administering to said patient a therapeutically effective amount of a compound according to Formula (I), or a stereoisomer, a tautomer, or a pharmaceutically acceptable salt thereof, wherein said cancer is breast cancer, including ductal carcinoma, invasive ductal carcinoma metastatic breast cancer, triple-negative breast cancer, human epidermal growth factor receptor 2 (HER2)-positive breast cancer, estrogen receptor (ER)-positive breast cancer, hormone receptor-positive breast cancer, and hormone receptor-negative breast cancer.

In one embodiment, a method is provided for the treatment of cancer, in a patient comprising administering to said patient a therapeutically effective amount of a compound according to Formula (I), or a stereoisomer, a tautomer, or a pharmaceutically acceptable salt thereof, wherein said cancer is prostate cancer, including adenocarcinoma of the prostate and castration-resistant prostate cancer.

In one embodiment, a method is provided for the treatment of cancer, in a patient comprising administering to said patient a therapeutically effective amount of a compound according to Formula (I), or a stereoisomer, a tautomer, or a pharmaceutically acceptable salt thereof, wherein said cancer is pancreatic cancer, including pancreatic adenocarcinoma, exocrine pancreatic cancer and neuroendocrine pancreatic cancer. In one embodiment, a method is provided for the treatment of cancer, in a patient comprising administering to said patient a therapeutically effective amount of a compound according to Formula (I), or a stereoisomer, a tautomer, or a pharmaceutically acceptable salt thereof, wherein said cancer is kidney cancer, including renal cell carcinoma, clear cell renal cell carcinoma, and non-clear cell renal cell carcinomas, papillary renal cell carcinoma, Wilms tumor, and renal sarcoma.

In one embodiment, a method is provided for the treatment of cancer, in a patient comprising administering to said patient a therapeutically effective amount of a compound according to Formula (I), or a stereoisomer, a tautomer, or a pharmaceutically acceptable salt thereof, wherein said cancer is gastric cancer, including gastric carcinoma.

In one embodiment, a method is provided for the treatment of cancer, in a patient comprising administering to said patient a therapeutically effective amount of a compound according to Formula (I), or a stereoisomer, a tautomer, or a pharmaceutically acceptable salt thereof, wherein said cancer is kidney cancer, including renal carcinoma and kidney parenchymal carcinoma.

In one embodiment, a method is provided for the treatment of cancer, in a patient comprising administering to said patient a therapeutically effective amount of a compound according to Formula (I), or a stereoisomer, a tautomer, or a pharmaceutically acceptable salt thereof, wherein said cancer is liver cancer, including hepatocellular carcinoma.

In one embodiment, a method is provided for the treatment of cancer, in a patient comprising administering to said patient a therapeutically effective amount of a compound according to Formula (I), or a stereoisomer, a tautomer, or a pharmaceutically acceptable salt thereof, wherein said cancer is ovarian cancer, including ovarian carcinoma.

In one embodiment, a method is provided for the treatment of cancer, in a patient comprising administering to said patient a therapeutically effective amount of a compound according to Formula (I), or a stereoisomer, a tautomer, or a pharmaceutically acceptable salt thereof, wherein said cancer is lymphoma, including Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia, and diffuse large B-cell lymphoma (DLBCL).

In one embodiment, a method is provided for the treatment of cancer, in a patient comprising administering to said patient a therapeutically effective amount of a compound according to Formula (I), or a stereoisomer, a tautomer, or a pharmaceutically acceptable salt thereof, wherein said cancer is leukemia, including acute lymphatic leukemia (ALL), chronic lymphatic leukemia (CLL), acute myeloid leukemia (AML), chronic myeloid leukemia (CML), adult T-cell leukemia lymphoma, and diffuse large B-cell lymphoma (DLBCL).

In one embodiment, a method is provided for the treatment of cancer, in a patient comprising administering to said patient a therapeutically effective amount of a compound according to Formula (I), or a stereoisomer, a tautomer, or a pharmaceutically acceptable salt thereof, wherein said cancer is multiple myeloma.

The compounds for Formula (I) and pharmaceutical compositions comprising at least one compound of Formula (I) are useful in treating or preventing any diseases or conditions that are associated with the activity of CDK2 proteins. Any method of administration may be used to deliver the compound or pharmaceutical composition to the patient. In certain embodiments, the compound of Formula (I) or pharmaceutical composition comprising at least compound of Formula (I) is administered orally. In other embodiments, the Formula (I) or pharmaceutical composition comprising at least compound of Formula (I) is administered parenterally.

In one embodiment, the present invention provides a combined preparation of a compound of Formula (I), and/or a pharmaceutically acceptable salt thereof; and additional therapeutic agent(s) for simultaneous, separate, or sequential use in the treatment and/or prophylaxis of multiple diseases or disorders associated with the activity of CDK2 protein. The combined preparation can be used to decrease the protein level, to decrease the protein activity level, and/or to inhibit the expression level of CDK2 proteins.

In one aspect, the compound(s) of Formula (I) are sequentially administered prior to administration of the immuno-oncology agent. In another aspect, compound(s) of Formula (I) are administered concurrently with the immuno-oncology agent. In yet another aspect, compound(s) of Formula (I) are sequentially administered after administration of the immuno-oncology agent.

In another aspect, compounds of Formula (I) may be co-formulated with an immuno-oncology agent.

Immuno-oncology agents include, for example, a small molecule drug, antibody, or other biologic or small molecule. Examples of biologic immuno-oncology agents include, but are not limited to, cancer vaccines, antibodies, and cytokines. In one aspect, the antibody is a monoclonal antibody. In another aspect, the monoclonal antibody is humanized or human.

In one aspect, the immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses (often referred to as immune checkpoint regulators).

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTBR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTβR, Lymphotoxin α 182, FAS, FASL, RELT, DR6, TROY, NGFR.

In one aspect, T cell responses can be stimulated by a combination of a compound of Formula (I) and one or more of (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4, and (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

Other agents that can be combined with compounds of Formula (I) for the treatment of cancer include antagonists of inhibitory receptors on NK cells or agonists of activating receptors on NK cells. For example, compounds of Formula (I) can be combined with antagonists of KIR, such as lirilumab.

Yet other agents for combination therapies include agents that inhibit or deplete macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357).

In another aspect, compounds of Formula (I) can be used with one or more of agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell anergy or exhaustion) and agents that trigger innate immune activation and/or inflammation at tumor sites.

In one aspect, the immuno-oncology agent is a CTLA-4 antagonist, such as an antagonistic CTLA-4 antibody. Suitable CTLA-4 antibodies include, for example, YERVOY (ipilimumab) or tremelimumab.

In another aspect, the immuno-oncology agent is a PD-1 antagonist, such as an antagonistic PD-1 antibody. Suitable PD-1 antibodies include, for example, OPDIVO (nivolumab), KEYTRUDA (pembrolizumab), MEDI-0680 (AMP-514; WO2012/145493), LIBTAYO(cemiplimab), JEMPERLI (dostarlimab), and ZYNYZ (retifanlimab) The immuno-oncology agent may also include pidilizumab (CT-011), though its specificity for PD-1 binding has been questioned. Another approach to target the PD-1 receptor is the recombinant protein composed of the extracellular domain of PD-L2 (B7-DC) fused to the Fc portion of IgG1, called AMP-224.

In another aspect, the immuno-oncology agent is a PD-L1 antagonist, such as an antagonistic PD-L1 antibody. Suitable PD-L1 antibodies include, for example, MPDL3280A (RG7446; WO2010/077634), durvalumab (MEDI4736), BMS-936559 (WO207/005874), MSB0010718C (WO2013/79174), TECENTRIQ (atezolizumab), and BAVENCIO(avelumab).

In another aspect, the immuno-oncology agent is a LAG-3 antagonist, such as an antagonistic LAG-3 antibody. Suitable LAG3 antibodies include, for example, relatlimab, or IMP-731 or IMP-321 (WO08/132601, WO09/44273).

In another aspect, the immuno-oncology agent is a CD137 (4-1BB) agonist, such as an agonistic CD137 antibody. Suitable CD137 antibodies include, for example, urelumab and PF-05082566 (WO12/32433).

In another aspect, the immuno-oncology agent is a GITR agonist, such as an agonistic GITR antibody. Suitable GITR antibodies include, for example, BMS-986153, BMS-986156, TRX-518 (WO06/105021, WO09/009116) and MK-4166 (WO11/028683).

In another aspect, the immuno-oncology agent is an IDO antagonist. Suitable IDO antagonists include, for example, INCB-024360 (WO206/122150, WO07/75598, WO08/36653, WO08/36642), indoximod, or NLG-919 (WO09/73620, WO09/1156652, WO11/56652, WO12/142237).

In another aspect, the immuno-oncology agent is an OX40 agonist, such as an agonistic OX40 antibody. Suitable OX40 antibodies include, for example, MEDI-6383 or MEDI-6469.

In another aspect, the immuno-oncology agent is an OX40L antagonist, such as an antagonistic OX40 antibody. Suitable OX40L antagonists include, for example, RG-7888 (WO06/029879).

In another aspect, the immuno-oncology agent is a CD40 agonist, such as an agonistic CD40 antibody. In yet another embodiment, the immuno-oncology agent is a CD40 antagonist, such as an antagonistic CD40 antibody. Suitable CD40 antibodies include, for example, lucatumumab or dacetuzumab.

In another aspect, the immuno-oncology agent is a CD27 agonist, such as an agonistic CD27 antibody. Suitable CD27 antibodies include, for example, varlilumab.

In another aspect, the immuno-oncology agent is MGA271 (to B7H3) (WO11/109400).

In another aspect, the immuno-oncology agent is an anti-TIGIT agent. Suitable anti-TIGIT agents include antibodies such as an BMS-986207, tiragolumab, or MK-7684.

In another aspect, the immuno-oncology agent is a KRAS G12C inhibitor. Suitable KRAS G12C inhibitors include LUMAKRAS (sotorasib) or KRAZATI (adagrasib).

The combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single dosage form having a fixed ratio of each therapeutic agent or in multiple, single dosage forms for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. Combination therapy also can embrace the administration of the therapeutic agents as described above in further combination with other biologically active ingredients and non-drug therapies (e.g., surgery or radiation treatment.) Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

One or more additional pharmaceutical agents or treatment methods such as, for example, chemotherapeutics or other anti-cancer agents, immune enhancers, immunosuppressants, radiation, anti-tumor vaccines, cytokine therapy (e.g., IL-2 and GM-CSF), and/or tyrosine kinase inhibitors can be optionally used in combination with the compounds of Formula (I) for treatment of CDK2 protein associated diseases, disorders or conditions. The agents can be combined with the present compounds in a single dosage form, or the agents can be administered simultaneously or sequentially as separate dosage forms.

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide (CYTOXAN®), ifosfamide, melphalan, chlorambucil, pipobroman, triethylene-melamine, triethylenethiophosphoramine, busulfan, carmustine, lomustine, streptozocin, dacarbazine, and temozolomide.

Suitable chemotherapeutic or other anti-cancer agents include, for example, antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine.

Suitable chemotherapeutic or other anti-cancer agents further include, for example, certain natural products and their derivatives (for example, vinca alkaloids, antitumor antibiotics, enzymes, lymphokines and epipodophyllotoxins) such as vinblastine, vincristine, vindesine, vinorelbine (Vavelbene®), bleomycin, dactinomycin, daunorubicin, doxorubicin, epirubicin, idarubicin, ara-C, paclitaxel (Taxol), mithramycin, deoxyco-formycin, mitomycin-C, L-asparaginase, interferons (especially IFN-alpha), etoposide, and teniposide.

Also suitable are cytotoxic agents such as epidophyllotoxin; an antineoplastic enzyme inhibitor; a topoisomerase inhibitor such as irinotecan (Camptosar®, CPT-11); procarbazine; mitoxantrone; platinum coordination complexes such as cisplatin and carboplatin; biological response modifiers; growth inhibitors; tegafur; capecitabine; and haematopoietic growth factors.

Other anti-cancer agents include antihormonal or endocrine therapeutic agents. Suitable endocrine therapeutic agents include, but are not limited to, aromatase inhibitors such as letrozole, anastrozole, and exemestane; selective estrogen receptor modulators (SERMs) such as tamoxifen, raloxifene, and droloxifene; and selective estrogen receptor degraders (SERDs) such as fulvestrant.

Other anti-cancer agents include antibody therapeutics such as pertuzumab, antibodies to costimulatory molecules such as CTLA-4, 4-1BB and PD-1, or antibodies to cytokines (IL-10 or TGF-β).

Other anti-cancer agents also include those that block immune cell migration such as antagonists to chemokine receptors, including CCR2 and CCR4.

Other anti-cancer agents also include those that augment the immune system such as adjuvants or adoptive T cell transfer.

Anti-cancer vaccines include dendritic cells, synthetic peptides, DNA vaccines and recombinant viruses.

The pharmaceutical composition of the invention may optionally include at least one signal transduction modulator (STM). A “signal transduction modulator” is an agent that selectively modulates one or more vital steps in signaling pathways, in the normal function of cancer cells, thereby leading to growth arrest and/or apoptosis. Suitable STMs include, but are not limited to: (i) bcr/abl kinase inhibitors such as, for example, STI 571 (GLEEVEC®); (ii) epidermal growth factor (EGF) receptor inhibitors such as, for example, kinase inhibitors (IRESSA®, SSI-774) and antibodies (Imclone: C225 [Goldstein et al., Clin. Cancer Res., 1:1311-1318 (1995)], and Abgenix: ABX-EGF); (iii) her-2/neu receptor inhibitors such as, for example, trastuzumab (Herceptin®) and farnesyl transferase inhibitors (FTI) such as, for example, L-744,832 (Kohl et al., Nat. Med., 1 (8): 792-797 (1995)); (iv) inhibitors of Akt family kinases or the Akt pathway, such as, for example, rapamycin (see, for example, Sekulic et al., Cancer Res., 60:3504-3513 (200)); (v) cell cycle kinase inhibitors such as, for example, palbociclib (Ibrance®), ribociclib (Kisqali®), and abemaciclib (Verzenio®) (see, for example, Jhaveri et al., Expert Rev. Anticancer Ther., 21 (10): 1105-1124 (2021)); and (vi) phosphatidyl inositol kinase inhibitors such as, for example, LY294002 (see, for example, Vlahos et al., J. Biol. Chem., 269:5241-5248 (1994)). Alternatively, at least one STM and at least one compound of Formula (I) may be in separate pharmaceutical compositions. In a specific embodiment of the present invention, at least one compound of Formula (I) and at least one STM may be administered to the patient concurrently or sequentially. In other words, at least one compound of Formula (I) may be administered first, at least one STM may be administered first, or at least one compound of Formula (I) and at least one STM may be administered at the same time. Additionally, when more than one compound of Formula (I) and/or STM is used, the compounds may be administered in any order.

In a specific embodiment of the present invention, at least one compound of Formula (I) and at least one chemotherapeutic agent are administered to the patient concurrently or sequentially. In other words, at least one compound of Formula (I) may be administered first, at least one chemotherapeutic agent may be administered first, or at least one compound of Formula (I) and the at least one STM may be administered at the same time. Additionally, when more than one compound of Formula (I) and/or chemotherapeutic agent is used, the compounds may be administered in any order. Similarly, any STM may also be administered at any point in comparison to the administration of the compound of Formula (I).

The combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single dosage form having a fixed ratio of each therapeutic agent or in multiple, single dosage forms for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. Combination therapy also can embrace the administration of the therapeutic agents as described above in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

### Pharmaceutical Compositions

The invention also provides pharmaceutical compositions which comprise a therapeutically effective amount of one or more of the compounds of Formula (I), formulated together with one or more pharmaceutically acceptable carriers (additives) and/or diluents, and optionally, one or more additional therapeutic agents described above.

The compounds of Formula (I) may be administered by any suitable route, preferably in the form of a pharmaceutical composition adapted to such a route, and in a dose effective for the treatment intended. The compounds and compositions of the compound of Formula (I) can be administered for any of the uses described herein by any suitable means, for example, orally, such as tablets, capsules (each of which includes sustained release or timed release formulations), pills, powders, granules, elixirs, tinctures, suspensions (including nanosuspensions, microsuspensions, spray-dried dispersions), syrups, and emulsions; sublingually; bucally; parenterally, such as by subcutaneous, intravenous, intramuscular, or intrasternal injection, or infusion techniques (e.g., as sterile injectable aqueous or non-aqueous solutions or suspensions); nasally, including administration to the nasal membranes, such as by inhalation spray; topically, such as in the form of a cream or ointment; or rectally such as in the form of suppositories. They can be administered alone, but generally will be administered with a pharmaceutical carrier selected on the basis of the chosen route of administration and standard pharmaceutical practice.

For oral administration, the pharmaceutical composition may be in the form of, for example, a tablet, capsule, liquid capsule, suspension, or liquid. The pharmaceutical composition is preferably made in the form of a dosage unit containing a particular amount of the active ingredient. For example, the pharmaceutical composition may be provided as a tablet or capsule comprising an amount of active ingredient in the range of from about 0.1 to 1000 mg, preferably from about 0.25 to 250 mg, and more preferably from about 0.5 to 100 mg. A suitable daily dose for a human or other mammal may vary widely depending on the condition of the patient and other factors, but can be determined using routine methods.

Any pharmaceutical composition contemplated herein can, for example, be delivered orally via any acceptable and suitable oral preparations. Exemplary oral preparations, include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the invention can contain at least one agent selected from sweetening agents, flavoring agents, coloring agents, demulcents, antioxidants, and preserving agents.

A tablet can, for example, be prepared by admixing at least one compound of Formula (I) and/or at least one pharmaceutically acceptable salt thereof with at least one non-toxic pharmaceutically acceptable excipient suitable for the manufacture of tablets. Exemplary excipients include, but are not limited to, for example, inert diluents, such as, for example, calcium carbonate, sodium carbonate, lactose, calcium phosphate, and sodium phosphate; granulating and disintegrating agents, such as, for example, microcrystalline cellulose, sodium crosscarmellose, corn starch, and alginic acid; binding agents, such as, for example, starch, gelatin, polyvinyl-pyrrolidone, and acacia; and lubricating agents, such as, for example, magnesium stearate, stearic acid, and talc. Additionally, a tablet can either be uncoated, or coated by known techniques to either mask the bad taste of an unpleasant tasting drug, or delay disintegration and absorption of the active ingredient in the gastrointestinal tract thereby sustaining the effects of the active ingredient for a longer period. Exemplary water soluble taste masking materials, include, but are not limited to, hydroxypropyl-methylcellulose and hydroxypropyl-cellulose. Exemplary time delay materials, include, but are not limited to, ethyl cellulose and cellulose acetate butyrate.

Hard gelatin capsules can, for example, be prepared by mixing at least one compound of Formula (I) and/or at least one salt thereof with at least one inert solid diluent, such as, for example, calcium carbonate; calcium phosphate; and kaolin.

Soft gelatin capsules can, for example, be prepared by mixing at least one compound of Formula (I) and/or at least one pharmaceutically acceptable salt thereof with at least one water soluble carrier, such as, for example, polyethylene glycol; and at least one oil medium, such as, for example, peanut oil, liquid paraffin, and olive oil.

An aqueous suspension can be prepared, for example, by admixing at least one compound of Formula (I) and/or at least one pharmaceutically acceptable salt thereof with at least one excipient suitable for the manufacture of an aqueous suspension. Exemplary excipients suitable for the manufacture of an aqueous suspension, include, but are not limited to, for example, suspending agents, such as, for example, sodium carboxymethylcellulose, methylcellulose, hydroxypropylmethyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example heptadecaethylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame. Oily suspensions can, for example, be prepared by suspending at least one compound of Formula (I) and/or at least one pharmaceutically acceptable salt thereof in either a vegetable oil, such as, for example, arachis oil; olive oil; sesame oil; and coconut oil; or in mineral oil, such as, for example, liquid paraffin. An oily suspension can also contain at least one thickening agent, such as, for example, beeswax; hard paraffin; and cetyl alcohol. In order to provide a palatable oily suspension, at least one of the sweetening agents already described hereinabove, and/or at least one flavoring agent can be added to the oily suspension. An oily suspension can further contain at least one preservative, including, but not limited to, for example, an anti-oxidant, such as, for example, butylated hydroxyanisol, and alpha-tocopherol.

Dispersible powders and granules can, for example, be prepared by admixing at least one compound of Formula (I) and/or at least one pharmaceutically acceptable salt thereof with at least one dispersing and/or wetting agent; at least one suspending agent;

and/or at least one preservative. Suitable dispersing agents, wetting agents, and suspending agents are as already described above. Exemplary preservatives include, but are not limited to, for example, anti-oxidants, e.g., ascorbic acid. In addition, dispersible powders and granules can also contain at least one excipient, including, but not limited to, for example, sweetening agents; flavoring agents; and coloring agents.

An emulsion of at least one compound of Formula (I) and/or at least one pharmaceutically acceptable salt thereof can, for example, be prepared as an oil-in-water emulsion. The oily phase of the emulsions comprising compounds of Formula (I) may be constituted from known ingredients in a known manner. The oil phase can be provided by, but is not limited to, for example, a vegetable oil, such as, for example, olive oil and arachis oil; a mineral oil, such as, for example, liquid paraffin; and mixtures thereof. While the phase may comprise merely an emulsifier, it may comprise a mixture of at least one emulsifier with a fat or an oil or with both a fat and an oil. Suitable emulsifying agents include, but are not limited to, for example, naturally-occurring phosphatides, e.g., soybean lecithin; esters or partial esters derived from fatty acids and hexitol anhydrides, such as, for example, sorbitan monooleate; and condensation products of partial esters with ethylene oxide, such as, for example, polyoxyethylene sorbitan monooleate.

Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. An emulsion can also contain a sweetening agent, a flavoring agent, a preservative, and/or an antioxidant. Emulsifiers and emulsion stabilizers suitable for use in the formulation of the present invention include Tween 60, Span 80, cetostearyl alcohol, myristyl alcohol, glyceryl monostearate, sodium lauryl sulfate, glyceryl distearate alone or with a wax, or other materials well known in the art.

The compounds of Formula (I) and/or at least one pharmaceutically acceptable salt thereof can, for example, also be delivered intravenously, subcutaneously, and/or intramuscularly via any pharmaceutically acceptable and suitable injectable form. Exemplary injectable forms include, but are not limited to, for example, sterile aqueous solutions comprising acceptable vehicles and solvents, such as, for example, water, Ringer's solution, and isotonic sodium chloride solution; sterile oil-in-water microemulsions; and aqueous or oleaginous suspensions.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers including saline, dextrose, or water, or with cyclodextrin (i.e., Captisol), cosolvent solubilization (i.e., propylene glycol) or micellar solubilization (i.e. Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid find use in the preparation of injectables.

A sterile injectable oil-in-water microemulsion can, for example, be prepared by 1) dissolving at least one compound of Formula (I) in an oily phase, such as, for example, a mixture of soybean oil and lecithin; 2) combining the Formula (I) containing oil phase with a water and glycerol mixture; and 3) processing the combination to form a microemulsion.

A sterile aqueous or oleaginous suspension can be prepared in accordance with methods already known in the art. For example, a sterile aqueous solution or suspension can be prepared with a non-toxic parenterally-acceptable diluent or solvent, such as, for example, 1,3-butane diol; and a sterile oleaginous suspension can be prepared with a sterile non-toxic acceptable solvent or suspending medium, such as, for example, sterile fixed oils, e.g., synthetic mono- or diglycerides; and fatty acids, such as, for example, oleic acid.

Pharmaceutically acceptable carriers are formulated according to a number of factors well within the purview of those of ordinary skill in the art. These include, without limitation: the type and nature of the active agent being formulated; the subject to which the agent-containing composition is to be administered; the intended route of administration of the composition; and the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example, Allen, L. V. Jr. et al. Remington: The Science and Practice of Pharmacy (2 Volumes), 22nd Edition (2012), Pharmaceutical Press.

Pharmaceutically acceptable carriers, adjuvants, and vehicles that may be used in the pharmaceutical compositions of this invention include, but are not limited to, ion exchangers, alumina, aluminum stearate, lecithin, self-emulsifying drug delivery systems (SEDDS) such as d-alpha-tocopherol polyethyleneglycol 1000 succinate, surfactants used in pharmaceutical dosage forms such as Tweens, polyethoxylated castor oil such as CREMOPHOR surfactant (BASF), or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkylcyclodextrins, including 2- and 3-hydroxypropyl-cyclodextrins, or other solubilized derivatives may also be advantageously used to enhance delivery of compounds of the formulae described herein.

The pharmaceutically active compounds of this invention can be processed in accordance with conventional methods of pharmacy to produce medicinal agents for administration to patients, including humans and other mammals. The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

For therapeutic purposes, the active compounds of this invention are ordinarily combined with one or more adjuvants appropriate to the indicated route of administration. If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

The amounts of compounds that are administered and the dosage regimen for treating a disease condition with the compounds and/or compositions of this invention depends on a variety of factors, including the age, weight, sex, the medical condition of the subject, the type of disease, the severity of the disease, the route and frequency of administration, and the particular compound employed. Thus, the dosage regimen may vary widely, but can be determined routinely using standard methods. A daily dose of about 0.001 to 100 mg/kg body weight, preferably between about 0.0025 and about 50 mg/kg body weight and most preferably between about 0.005 to 10 mg/kg body weight, may be appropriate. The daily dose can be administered in one to four doses per day. Other dosing schedules include one dose per week and one dose per two day cycle.

Pharmaceutical compositions of this invention comprise at least one compound of Formula (I) and/or at least one pharmaceutically acceptable salt thereof, and optionally an additional agent selected from any pharmaceutically acceptable carrier, adjuvant, and vehicle. Alternate compositions of this invention comprise a compound of the Formula (I) described herein, or a prodrug thereof, and a pharmaceutically acceptable carrier, adjuvant, or vehicle.

The present invention also includes pharmaceutical kits useful, for example, in the treatment or prevention of CDK2 protein-associated diseases or disorders, and other diseases referred to herein which include one or more containers containing a pharmaceutical composition comprising a therapeutically effective amount of a compound of Formula (I). Such kits can further include, if desired, one or more of various conventional pharmaceutical kit components, such as, for example, containers with one or more pharmaceutically acceptable carriers, additional containers, as will be readily apparent to those skilled in the art. Instructions, either as inserts or as labels, indicating quantities of the components to be administered, guidelines for administration, and/or guidelines for mixing the components, can also be included in the kit.

The dosage regimen for the compounds of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired.

By way of general guidance, the daily oral dosage of each active ingredient, when used for the indicated effects, will range between about 0.001 to about 5000 mg per day, preferably between about 0.01 to about 1000 mg per day, and most preferably between about 0.1 to about 250 mg per day. Intravenously, the most preferred doses will range from about 0.01 to about 10 mg/kg/minute during a constant rate infusion. Compounds of Formula (I) may be administered in a single daily dose, or the total daily dosage may be administered in divided doses of two, three, or four times daily.

The compounds are typically administered in admixture with suitable pharmaceutical diluents, excipients, or carriers (collectively referred to herein as pharmaceutical carriers) suitably selected with respect to the intended form of administration, e.g., oral tablets, capsules, elixirs, and syrups, and consistent with conventional pharmaceutical practices.

Dosage forms (pharmaceutical compositions) suitable for administration may contain from about 1 milligram to about 200 milligrams of active ingredient per dosage unit. In these pharmaceutical compositions the active ingredient will ordinarily be present in an amount of about 0.1-95% by weight based on the total weight of the composition.

A typical capsule for oral administration contains at least one of the compounds of Formula (I) (250 mg), lactose (75 mg), and magnesium stearate (15 mg). The mixture is passed through a 60 mesh sieve and packed into a No. 1 gelatin capsule.

A typical injectable preparation is produced by aseptically placing at least one of the compounds of Formula (I) (250 mg) into a vial, aseptically freeze-drying and sealing. For use, the contents of the vial are mixed with 2 mL of physiological saline, to produce an injectable preparation.

The present invention includes within its scope pharmaceutical compositions comprising, as an active ingredient, a therapeutically effective amount of at least one of the compounds of Formula (I), alone or in combination with a pharmaceutical carrier. Optionally, compounds of Formula (I) can be used alone, in combination with other compounds of Formula (I), or in combination with one or more other therapeutic agent(s), e.g., an anticancer agent or other pharmaceutically active material.

Regardless of the route of administration selected, the compounds of Formula (I), which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the therapeutic response for a particular patient, composition, and mode of administration, without being toxic to the patient.

The selected dosage level will depend upon a variety of factors including the activity of the particular compound of Formula (I) employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of Formula (I) employed in the pharmaceutical composition at levels lower than that required in order to achieve the therapeutic effect and gradually increase the dosage until the effect is achieved.

In general, a suitable daily dose of a compound of Formula (I) will be that amount of the compound which is the lowest dose effective to produce a therapeutic effect. Such an effective dose will generally depend upon the factors described above. Generally, oral, intravenous, intracerebroventricular and subcutaneous doses of the compounds of Formula (I) for a patient will range from about 0.01 to about 50 mg per kilogram of body weight per day.

If desired, the effective daily dose of the active compound may be administered as two, three, four, five, six or more sub-doses administered separately at appropriate intervals throughout the day, optionally, in unit dosage forms. In certain aspects of the invention, dosing is one administration per day.

While it is possible for a compound of Formula (I) to be administered alone, it is preferable to administer the compound as a pharmaceutical formulation (composition).

The above other therapeutic agents, when employed in combination with the compounds of Formula (I), may be used, for example, in those amounts indicated in the Physicians' Desk Reference (PDR) or as otherwise determined by one of ordinary skill in the art. In the methods of the present invention, such other therapeutic agent(s) may be administered prior to, simultaneously with, or following the administration of the inventive compounds.

### Methods of Preparation

The compounds of the present invention can be prepared in a number of ways well known to one skilled in the art of organic synthesis. The compounds of the present invention can be synthesized using the methods described below, together with synthetic methods know in the art of synthetic organic chemistry, or variations thereon as appreciated by those skilled in the art. Preferred methods include, but are not limited to, those described below. All references cited herein are hereby incorporated by reference in their entirety.

The compounds of this invention may be prepared using the reactions and techniques described in this section. The reactions are performed in solvents appropriate to the reagents and materials employed and are suitable for the transformations being effected. Also, in the description of the synthetic methods described below, it is to be understood that all proposed reaction conditions, including choice of solvent, reaction atmosphere, reaction temperature, duration of the experiment and work up procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents that are compatible with the reaction conditions will be readily apparent to one skilled in the art and alternate methods must then be used. This will sometimes require a judgment to modify the order of the synthetic steps or to select one particular process scheme over another in order to obtain a desired compound of the invention. It will also be recognized that another major consideration in the planning of any synthetic route in this field is the judicious choice of the protecting group used for protection of the reactive functional groups present the compounds described in this invention. An authoritative account describing the many alternatives to the trained practitioner is Wuts, P. G.; Greene, T. W. Greene's Protective Groups in Organic Synthesis, 4th ed.; Wiley-Interscience: Hoboken, N.J., 2007.

### EXAMPLES

The following examples illustrate the particular embodiments of the present invention and do not limit the scope of the present invention. Chemical abbreviations and symbols as well as scientific abbreviations and symbols have their usual and customary meanings unless otherwise specified. Additional abbreviations employed in the Examples and elsewhere in this application are defined above. Common intermediates are generally useful for the preparation of more than one Example. Compounds of the Examples are identified by the example and step in which they were prepared (e.g., “1-A” denotes the Example 1, step A), or by the example only where the compound is the title compound of the example (for example, “1” denotes the title compound of Example 1). In some instances, alternate preparations of intermediates or examples are described. Frequently chemists skilled in the art of synthesis may devise alternative preparations which may be desirable based on one or more considerations such as shorter reaction time, less expensive starting materials, ease of operation or isolation, improved yield, amenable to catalysis, avoidance of toxic reagents, accessibility of specialized instrumentation, and decreased number of linear steps, etc. The intent of describing alternative preparations is to further enable the preparation of the examples of this invention. In some instances, some functional groups in the outlined examples and claims may be replaced by well-known bioisosteric replacements known in the art, for example, replacement of a carboxylic acid group with a tetrazole or a phosphate moiety.

### Materials and Methods

All reagents and dried solvents were purchased from commercial suppliers and used without further purification unless otherwise stated. Flash column chromatography on silica gel were performed on a Biotage Isolera Four or a Grace Reverleris X2 automated systems, using prepacked silica RediSep or Interchim puriFlash columns. Reversed-phase chromatography was performed on a Combi Flash Rf automated system, using prepacked C18 aqueous RediSep Rf columns.

### NMR Spectroscopy:

Sample preparation: Powders were solubilized in DMSO-d₆, vortexed vigorously until the solution was clear and transferred to an NMR tube for data acquisition.

Liquid-state NMR experiments were recorded on a 600 MHz (14.1 Tesla) Bruker Avance III NMR spectrometer (600 MHz for 1H, 151 MHz for ¹³C) using a triple-resonance ¹H/¹⁹F, ¹⁵N, ¹³C TR TCI 5 mm cryoprobe (Bruker Biospin, Germany). Liquid-state NMR experiments were recorded on a 500 MHz (11.75 Tesla) Bruker Avance NEO NMR spectrometer (500 MHz for 1H, 125 MHz for ¹³C) using a Dual Resonance 5 mm BBFO BBF/H iProbe (Bruker Biospin, Germany).

Liquid-state NMR experiments were recorded on a 400 MHz (9.4 Tesla) Bruker Avance NEO NMR spectrometer (400 MHz for 1H, 100 MHz for ¹³C) using a SEI 5 mm probe (Bruker Biospin, Germany).

All the experiments used for the resonance assignment procedure and the elucidation of the products structure (1D ¹H, 2D ¹H-¹H-COSY, 2D ¹H-¹H-ROESY, 2D 1H-¹³C-HSQC, 2D 1H-¹³C-HMBC) were recorded at 300 K. 1H chemical shifts are reported in (ppm) as s (singlet), d (doublet), t (triplet), q (quartet), dd (doublet of doublets), m (multiplet) or br s (broad singlet).

### Lcms Analysis:

Chemicals for analysis: All reagents used were analytical or ultra gradient grade.

Sample preparation: Powders were solubilized in DMSO or MeOH or Acetonitrile or water, or other solvents depending on their solubility, at the concentration around 0.5 mg/mL, vortexed vigorously until the solution was clear and transferred to a LCMS vial for data acquisition.

Two different LC-UV-MS instruments have been used. 1. Waters Acquity UPLC system equipped with Waters Acquity PDA e-detector coupled with a Waters simple quadruple detector SQD mass spectrometer. 2. Waters HClass equipped with an UPLC eLAMBDA PDA detector coupled with a Waters simple quadruple detector SQD mass spectrometer.

System 1 was configured in acidic reverse phase conditions. Chromatographic separations were performed with a Waters column Acquity UPLC® CSH™ C18 (2.1×50 mm) 1.7 μm-Eluent A=H₂O+0.02% HCOOH-Eluent B=CH₃CN+0.02% HCOOH-Oven T° C.: 55° C.-Gradient: t0 2% B, t4 min 98% B, t4.5 min 98% B, t4.6 min 2% B, t5.0 min 2% B-Flow rate=1 mL/min or with a Waters column Acquity UPLC® CSH™ C18 (2.1×100 mm) 1.7 μm-Eluent A=H₂O+0.02% HCOOH-Eluent B=CH₃CN+0.02% HCOOH-Oven T° C.: 55° C.-Gradient: t0 2% B, t15 min 98% B, t15.2 min 2% B, t18 min 2% B-Flow rate=0.7 mL/min. The injection volume is set from 0.5 μL to 2 μL; The UV chromatograms were recorded at a wavelength of 220 nm. The mass spectrometer was operated with a source configured in positive or negative electrospray ion mode. The source parameters were source temperature 150° C.; desolvatation temperature 250° C.; cone gas flow 150 (L/h); desolvation gas flow 1000 (L/h); Capillary: 3 kV-Sample cone: 15/30 V-Mass range: 120-1500 amu. Data acquisition and data reprocessing were performed using Masslynx software (Waters).

System 2 was configured as well as in acidic reverse-phase conditions, neutral reverse-phase conditions, or basic reverse phase conditions. Chromatographic separations were performed as follow: Waters Column Acquity UPLC CSH C18 (2.1×50 mm) 1.7 μm-Eluent A=H₂O+0.02% HCOOH-Eluent B=CH₃CN+0.02% HCOOH-Oven T° C.: 55° C.-Gradient: t0 2% B, t4 min 98% B, t4.5 min 98% B, t4.6 min 2% B, t5.5 min 2% B-Flow rate=1 mL/min or Waters Column Acquity UPLC CSH C18 (2.1×10⁰ mm) 1.7 μm-Eluent A=H₂O+0.02% HCOOH-Eluent B=CH₃CN+0.02% HCOOH-Oven T° C.: 55° C.-Gradient: t0 2% B, t15 min 98% B, t15.2 min 2% B, t18 min 2% B-Flow rate=0.7 mL/min or Waters Column Acquity UPLC CSH C18 (2.1×50 mm) 1.7 μm-Eluent A=H₂O+0.05% TFA-Eluent B=CH₃CN+0.035% TFA-Oven T° C.: 55° C.-Gradient: t0 2% B, t4 min 98% B, t4.5 min 98% B, t4.6 min 2% B, t5.5 min 2% B-Flow rate=1 mL/min or Waters Column Acquity UPLC CSH C18 (2.1×10⁰ mm) 1.7 μm-Eluent A=H₂O+0.05% TFA-Eluent B=CH₃CN+0.035% TFA-Oven T° C.: 55° C.-Gradient: t0 2% B, t15 min 98% B, t15.2 min 2% B, t18 min 2% B-Flow rate=0.7 mL/min or Waters Column Acquity UPLC BEH C18 (2.1×50 mm) 1.7 μm-Eluent A=H₂O+AcONH₄10 mM (Ammoniac in Water adjusted to pH7 with AcOH)-Eluent B=CH₃CN-Oven T° C.: 45° C.-Gradient: t0 2% B, t4 min 90% B, t4.5 min 90% B, t4.6 min 2% B, t5.5 min 2% B-Flow rate=0.8 mL/min or Waters Column Acquity UPLC BEH C18 (2.1×50 mm) 1.7 μm-Eluent A=H₂O+NH₄HCO₃ at 2 mM adjusted to pH10 with NaOH-Eluent B=CH₃CN-Oven T° C.: 45° C.-Gradient: t0 2% B, t4 min 90% B, t4.5 min 90% B, t4.6 min 2% B, t5.5 min 2% B-Flow rate=0.8 mL/min. The injection volume is set from 0.5 μL to 2 μL; The UV chromatograms were recorded at a wavelength of 220 nm. The mass spectrometer was operated with a source configured in positive or negative electrospray ion mode. The source parameters were source temperature 150° C.; desolvatation temperature 250° C.; cone gas flow 150 (L/h); desolvatation gas flow 1000 (L/h); Capillary: 3 kV-Sample cone: 15/30 V-Mass range: 120-1500 amu. Data acquisition and data reprocessing were performed using Masslynx software (Waters).

### LC/HRMS Analysis:

The instrument for LC/HRMS analysis is a Waters Acquity UPLC system equipped with Waters Acquity PDA coupled with a Waters Xevo-G2-XS-QTOF mass spectrometer.

The System is configured in acidic reverse-phase conditions. Chromatographic separations were performed with a Waters column Cortecs® UPLC® C18 (2.1×10⁰ mm) 1.6 μm-Eluent A=H₂O+0.05% TFA-Eluent B=CH₃CN+0.035% TFA-Oven T° C.: 55° C.-Gradient: t0 2% B, t15 min 98% B, t15.2 min 2% B, t18 min 2% B-Flow rate=0.7 mL/min. The injection volume is set from 0.5 μL to 2 μL; The UV chromatograms were recorded at a wavelength of 220 nm. The mass spectrometer was operated with a source configured in positive or negative electrospray ion mode. The source parameters were Analyzer mode: Sensitivity; source temperature 120° C.; desolvatation temperature 550° C.; cone gas flow 50 (L/h); desolvatation gas flow 1200 (L/h); Collision Energy 6V Capillary: 3 kV-Sample cone: 40 V-Source offset 80-Mass range: 120-1500 amu, scan time: 0.4 sec. The mass spectrometer is calibrated with sodium formate (Waters service Kit Part. No. 186007091-4). The lock mass used is Leucine enkephaline at 200 μg/μL, infused at 10 μL/min. The lock spray setup parameters are MS mode, single ion (556.2771 Da), sensitivity mode, scan time: 0.2 sec; interval 30 sec; scans to average: 3; mass windows: 0.5 Da (+/−). Data acquisition and data reprocessing were performed using Masslynx software (Waters).

### ABBREVIATIONS

AcOH acetic acid
AIBN azobisisobutyronitrile
aq. aqueous
Boc tert-butoxycarbonyl
B₂pin₂ bis(pinacolato)diboron
Brettphos-Pd-G1 chloro[2-(dicyclohexylphosphino)-3,6-dimethoxy-2′,4′,6′-triisopropyl-1,1′-biphenyl][2-(2-aminoethyl)phenyl]palladium(II)
Brettphos-Pd-G3 [(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate
CPME cyclopentyl methyl ether
DAST (diethylamino) sulfur trifluoride
DBAD di-tert-butyl azodicarboxylate
DCM dichloromethane
DCE 1,2-dichloroethane
DIAD diisopropyl azodicarboxylate
DIPEA N,N-diisopropylethylamine
DMAP N,N-dimethylpyridin-4-amine
DMF N,N-dimethylformamide
DMSO dimethylsulfoxide
Et₂O diethyl ether
EtOAc ethyl acetate
EtOH ethanol
FastWorX® trademark of hydrophobic powder
(g) gas
g gram(s)
h hour(s)
HATU 1-[Bis(dimethylamino)methylene]-1H-1,2,3-triazolo[4,5-b]pyridinium 3-oxide hexafluorophosphate
isopropylamine
iPrNH₂
iPrOH isopropanol
iPrOAc isopropyl acetate
KOAc potassium acetate
MeOH methanol
MeTHF methyl tetrahydrofuran
mg milligram(s)
min minute(s)
mL milliliter(s)
mmol millimole(s)
mol mole(s)
MTBE methyl tert-butyl ether
NMR nuclear magnetic resonance
p. purity
Pd₂(dba)₃ tris(dibenzylideneacetone)dipalladium(0)
Pd(dppf)Cl₂ [1,1′-bis(diphenylphosphino)ferrocene]dichloropalladium(II)
Pd(OAc)₂ palladium(II) acetate
Pd(PPh₃)₄ tetrakis(triphenylphosphine) palladium(0)
quant. quantitative
TBAF tetrabutylammonium fluoride
t-BuONa sodium tert-butoxide
TFA trifluoroacetic acid
THF tetrahydrofuran
T3P propane phosphonic acid anhydride
XantPhos (9,9-dimethyl-9H-xanthene-4,5-diyl)bis(diphenylphosphane)
XPhos-Pd-G2 chloro(2-dicyclohexylphosphino-2′,4′,6′-triisopropyl-1,1′-biphényl) [2-(2′-amino-1,1′-biphényl)]palladium(II)
y. yield
μL microliter(s)

### INTERMEDIATE BB-01

### 2-[(1S)-4-tert-butoxy-1-carbamoyl-4-oxo-butyl]-1-oxo-isoindoline-5-carboxylic acid

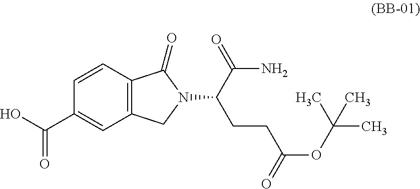 [SMILES: CC(C)(C)OC(=O)CC[C@@H](C(N)=O)N1Cc2cc(C(=O)O)ccc2C1=O]

### Intermediate BB-01A: tert-butyl(4S)-5-amino-4-(5-bromo-1-oxo-isoindolin-2-yl)-5-oxo-pentanoate

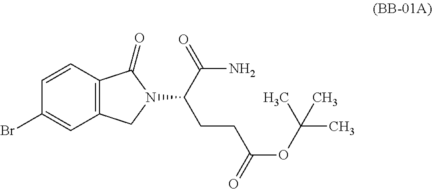 [SMILES: CC(C)(C)OC(=O)CC[C@@H](C(N)=O)N1Cc2cc(Br)ccc2C1=O]

To a stirred suspension of tert-butyl(4S)-4-amino-4-carbamoylbutanoate hydrochloride (4.26 g, 17.9 mmol) in acetonitrile (58 mL) was added DIPEA (8.5 mL, 48.7 mmol). The reaction mixture was stirred for 5 min. Next, methyl 4-bromo-2-(bromomethyl)benzoate (5.00 g, 16.2 mmol) was added. The reaction mixture was stirred at ambient temperature for 1 h and then at 70° C. overnight. The mixture was cooled to ambient temperature then diluted with EtOAc. The organic layer was washed with a 1 M aqueous solution of HCl (2×) then brine, dried over Na₂SO₄, filtered, and concentrated under reduced pressure. The crude was purified by flash chromatography on silica gel using a gradient of EtOAc in cyclohexane from 40% to 100% to afford tert-butyl(4S)-5-amino-4-(5-bromo-1-oxo-isoindolin-2-yl)-5-oxo-pentanoate as an off-white solid (5.33 g, 100% p., 83% y.). m/z [M+H]⁺=397.1, ¹H NMR (400 MHz, DMSO-d₆): δ (ppm) 7.90-7.85 (m, 1H), 7.71-7.60 (m, 2H), 7.58 (s, 1H), 7.19 (s, 1H), 4.80-4.68 (m, 1H), 4.61 (d, J=17.9 Hz, 1H), 4.47 (d, J=17.9 Hz, 1H), 2.24-2.07 (m, 3H), 2.07-1.87 (m, 1H), 1.32 (s, 9H).

### Intermediate BB-01

In sealed vial, to a stirred solution of tert-butyl(4S)-5-amino-4-(5-bromo-1-oxo-isoindolin-2-yl)-5-oxo-pentanoate (5.33 g, 13.4 mmol) in DMF (45 mL) under argon were added successively ethanedioic acid dihydrate (2.54 g, 20.1 mmol), XantPhos (388 mg, 0.671 mmol), and Pd(OAc)₂(152 mg, 0.671 mmol). The reaction mixture was degassed with argon for 5 min. Acetic anhydride (1.9 mL, 20.1 mmol) and DIPEA (3.5 mL, 20.1 mmol) were added. The reaction mixture was stirred at 100° C. for 4 h. The reaction mixture was cooled to ambient temperature then concentrated under reduced pressure. The reaction mixture was poured into a saturated aqueous solution of NaHCO₃. The aqueous layer was washed with EtOAc (2×). The aqueous layer was acidified to pH 3-4 with a 1 M aqueous solution of HCl then extracted with EtOAc (3×). The combined organic layers were dried over Na₂SO₄, filtered, and concentrated under reduced pressure to afford 2-[(1S)-4-tert-butoxy-1-carbamoyl-4-oxo-butyl]-1-oxo-isoindoline-5-carboxylic acid as a pale yellow foam (4.35 g, 81% p., 73% y.). m/z [M+H]⁺=363.3, ¹H NMR (400 MHz, DMSO-d₆): δ (ppm) 12.99 (s, 1H), 8.17 (t, J=1.1 Hz, 1H), 8.05 (dd, J=7.9, 1.4 Hz, 1H), 7.80 (dd, J=7.9, 0.7 Hz, 1H), 7.60 (d, J=12.5 Hz, 1H), 7.21 (s, 1H), 4.82-4.71 (m, 1H), 4.66 (d, J=17.9 Hz, 1H), 4.53 (d, J=17.9 Hz, 1H), 2.17 (t, J=4.1 Hz, 3H), 2.07-1.94 (m, 1H), 1.32 (s, 9H).

### INTERMEDIATE BB-02

### 2-[(1S)-4-tert-butoxy-1-carbamoyl-4-oxo-butyl]-4-methyl-1-oxo-isoindoline-5-carboxylic acid

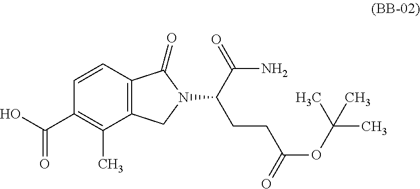 [SMILES: Cc1c(C(=O)O)ccc2c1CN([C@@H](CCC(=O)OC(C)(C)C)C(N)=O)C2=O]

### Intermediate BB-02A: tert-butyl(4S)-5-amino-4-(5-bromo-4-methyl-1-oxo-isoindolin-2-yl)-5-oxo-pentanoate

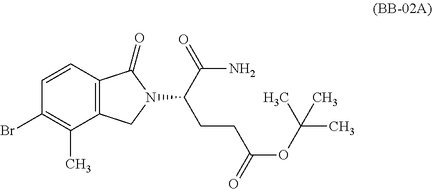 [SMILES: Cc1c(Br)ccc2c1CN([C@@H](CCC(=O)OC(C)(C)C)C(N)=O)C2=O]

In a sealed vial, to a stirred suspension of tert-butyl(4S)-4-amino-4-carbamoylbutanoate hydrochloride (313 mg, 1.31 mmol) in dry acetonitrile (2.0 mL) at ambient temperature was added DIPEA (458 μL, 2.62 mmol). The reaction mixture was stirred for 30 min. A solution of methyl 4-bromo-2-(bromomethyl)-3-methyl-benzoate (621 mg, 0.656 mmol) in dry acetonitrile (1.5 mL) was added. The reaction mixture was stirred at 80° C. for 16 h. The reaction mixture was poured into water and the aqueous layer was extracted with EtOAc (3×). The combined organic layers were washed with brine, dried over Na₂SO₄, filtered, and concentrated under reduced pressure. The crude material was purified by flash chromatography on silica gel using a gradient of EtOAc/EtOH (3/1) in n-heptane from 1% to 50%. Fractions were combined and concentrated under reduced pressure. The residue was solubilized in a minimum amount of DCM and the solution was added dropwise to stirred solution of n-pentane. The resulting mixture was stirred for 30 min. The suspension was filtered, washed with n-pentane, and dried under reduced pressure to afford tert-butyl(4S)-5-amino-4-(5-bromo-4-methyl-1-oxo-isoindolin-2-yl)-5-oxo-pentanoate as a pale yellow solid (204 mg, 99% p., 75% y.). m/z [M+Na]⁺=435.3, ¹H NMR (DMSO-d₆): δ (ppm) 7.72 (d, J=8.0 Hz, 1H), 7.55 (s, 1H), 7.45 (d, 8.0 Hz, 1H), 7.19 (s, 1H), 4.73 (dd, J=9.4, 3.5 Hz, 1H), 4.59 (d, J=17.7 Hz, 1H), 4.49 (d, J=17.8 Hz, 1H), 2.36 (s, 3H), 2.23-2.10 (m, 3H), 2.06-1.94 (m, 1H), 1.34 (s, 9H).

### Intermediate BB-02

In an autoclave, to a stirred solution of tert-butyl(4S)-5-amino-4-(5-bromo-4-methyl-1-oxo-isoindolin-2-yl)-5-oxo-pentanoate (204 mg, 0.491 mmol) in dry DMF (1.7 mL) under argon were added successively ethanedioic acid dihydrate (93.0 mg, 0.740 mmol), Xantphos (14.0 mg, 0.025 mmol) and Pd(OAc)₂(5.7 mg, 0.025 mmol). The reaction mixture was degassed with argon for 5 min then acetic anhydride (68.0 μL, 0.740 mmol) and DIPEA (129 μL, 0.737 mmol) were added. The reaction mixture was stirred at 100° C. overnight. The reaction was quenched with the addition of saturated aqueous solution of NaHCO₃. EtOAc was added to the mixture. The phases were separated, and the aqueous layer washed with EtOAc. The aqueous layer was acidified to pH 3-4 with a 1 M aqueous solution of HCl and extracted with EtOAc (2×). The combined organic layers were washed with brine, dried over Na₂SO₄, filtered, and concentrated under reduced pressure. The residue was triturated with n-pentane and stirred for 30 min. The suspension was filtered, washed with n-pentane, and dried under reduced pressure to afford 2-[(1S)-4-tert-butoxy-1-carbamoyl-4-oxo-butyl]-4-methyl-1-oxo-isoindoline-5-carboxylic acid as a white solid (126 mg, 95% p., 65% y.). m/z [M+Na]⁺=399.4, ¹H NMR (DMSO-d₆): δ (ppm) 13.17 (s, 1H), 7.89 (d, J=7.8 Hz, 1H), 7.59 (d, J=7.9 Hz, 1H), 7.56 (s, 1H), 7.20 (s, 1H), 4.79-4.71 (m, 1H), 4.59 (d, J=17.6 Hz, 1H), 4.50 (d, J=17.6 Hz, 1H), 2.49 (s, 3H), 2.26-2.11 (m, 3H), 2.08-1.97 (m, 1H), 1.34 (s, 9H).

### INTERMEDIATE BB-03 2-[(1S)-4-tert-butoxy-1-carbamoyl-4-oxo-butyl]-6-fluoro-1-oxo-isoindoline-5-carboxylic acid

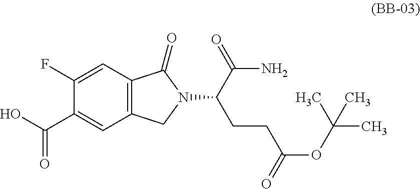 [SMILES: CC(C)(C)OC(=O)CC[C@@H](C(N)=O)N1Cc2cc(C(=O)O)c(F)cc2C1=O]

### Intermediate BB-03A: methyl 4-bromo-2-(bromomethyl)-5-fluoro-benzoate

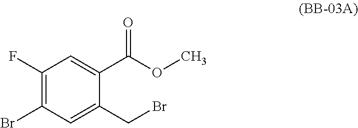

To a stirred solution of methyl 4-bromo-5-fluoro-2-methylbenzoate (4.05 g, 16.4 mmol) in acetonitrile (55 mL) were added 1-bromopyrrolidine-2,5-dione (3.21 g, 18.0 mmol) and AIBN (269 mg, 1.64 mmol). The reaction mixture was irradiated with a white lamp (6200 K) for 2 h. The mixture was concentrated under reduced pressure. The crude was purified by flash chromatography on silica gel using a gradient of EtOAc in cyclohexane from 2% to 20% to afford methyl 4-bromo-2-(bromomethyl)-5-fluoro-benzoate as a yellow oil (5.13 g, 84% p., 81% y.). m/z [M+H]⁺=no ionization, ¹H NMR (400 MHz, DMSO-d₆): δ (ppm) 8.05 (d, J=6.8 Hz, 1H), 7.79 (d, J=9.3 Hz, 1H), 4.97 (s, 2H), 3.88 (s, 3H).

### Intermediate BB-03B: tert-butyl(4S)-5-amino-4-(5-bromo-6-fluoro-1-oxo-isoindolin-2-yl)-5-oxo-pentanoate

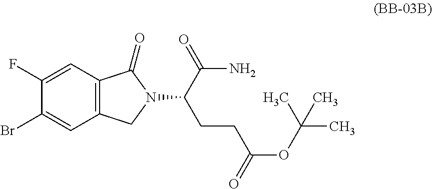 [SMILES: CC(C)(C)OC(=O)CC[C@@H](C(N)=O)N1Cc2cc(Br)c(F)cc2C1=O]

To a stirred suspension of tert-butyl(4S)-4-amino-4-carbamoylbutanoate hydrochloride (3.98 g, 16.7 mmol) in acetonitrile (26 mL) at 0° C. was added DIPEA (5.5 mL, 31.5 mmol) dropwise. The reaction mixture was stirred for 30 min at 0° C. A solution of methyl 4-bromo-2-(bromomethyl)-5-fluoro-benzoate (5.13 g, 15.7 mmol) in acetonitrile (45 mL) was added dropwise over 30 min at 0° C. The reaction mixture was then stirred at 70° C. overnight. The mixture was cooled to ambient temperature then concentrated under reduced pressure. The residue was triturated in water for 1 h, filtered, washed with water, and dried under reduced pressure. The residue was triturated in a mixture of n-pentane/EtOAc (5/1) for 1 h, filtered, washed with n-pentane and dried under reduced pressure at 50° C. overnight to afford tert-butyl(4S)-5-amino-4-(5-bromo-6-fluoro-1-oxo-isoindolin-2-yl)-5-oxo-pentanoate as an off-white solid (5.19 g, 99% p., 79% y.). m/z [M+H]⁺=415.1, ¹H NMR (400 MHz, DMSO-d₆): δ (ppm) 8.03 (d, J=6.0 Hz, 1H), 7.66 (d, J=7.7 Hz, 1H), 7.59 (s, 1H), 7.21 (s, 1H), 4.72 (dd, J=9.6, 3.8 Hz, 1H), 4.60 (d, J=17.7 Hz, 1H), 4.45 (d, J=17.7 Hz, 1H), 2.22-2.07 (m, 3H), 2.05-1.90 (m, 1H), 1.33 (s, 9H).

### Intermediate BB-03

In an autoclave, to a stirred solution of tert-butyl(4S)-5-amino-4-(5-bromo-6-fluoro-1-oxo-isoindolin-2-yl)-5-oxo-pentanoate (5.18 g, 12.5 mmol) in DMF (25 mL) under argon were added successively ethanedioic acid dihydrate (2.36 g, 18.7 mmol), XantPhos (361 mg, 0.624 mmol), and Pd(OAc)₂(141 mg, 0.624 mmol). The reaction mixture was degassed with argon for 5 min. Next, acetic anhydride (1.7 mL, 18.7 mmol) and DIPEA (3.3 mL, 18.7 mmol) were added. The reaction mixture was stirred at 100° C. for 4 h. The reaction mixture was cooled to ambient temperature. The reaction mixture was poured into a 1 M aqueous solution of NaHCO₃. The aqueous layer was washed with EtOAc. The aqueous layer was acidified to pH 3-4 with a concentrated aqueous solution of HCl, and then extracted with EtOAc (3×). The combined organic layers were dried using a phase separator and concentrated under reduced pressure. The residue was triturated in DCM. MTBE was added and the residue was triturated for 2 h. The suspension was filtered, washed with MTBE, and dried under reduced pressure to afford 2-[(1S)-4-tert-butoxy-1-carbamoyl-4-oxo-butyl]-6-fluoro-1-oxo-isoindoline-5-carboxylic acid as an off-white solid (2.96 g, 96% p., 60% y.). m/z [M+H]⁺=381.4, ¹H NMR (400 MHz, DMSO-d₆): δ (ppm) 13.56 (s, 1H), 8.09 (d, J=6.1 Hz, 1H), 7.67-7.52 (m, 2H), 7.24 (s, 1H), 4.80-4.69 (m, 1H), 4.69-4.41 (m, 2H), 2.24-2.08 (m, 3H), 2.08-1.87 (m, 1H), 1.33 (s, 9H).

### INTERMEDIATE BB-04 2-[(1S)-4-tert-butoxy-1-carbamoyl-4-oxo-butyl]-6-methyl-1-oxo-isoindoline-5-carboxylic acid

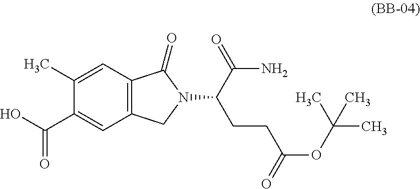 [SMILES: Cc1cc2c(cc1C(=O)O)CN([C@@H](CCC(=O)OC(C)(C)C)C(N)=O)C2=O]

### Intermediate BB-04A: methyl 4-bromo-2,5-dimethyl-benzoate

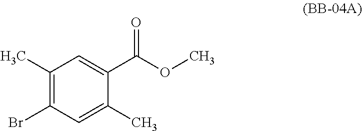

To a stirred solution of 4-bromo-2,5-dimethylbenzoic acid (10.0 g, 41.5 mmol) in MeOH (130 mL) at 25° C. was added sulfuric acid (442 μL, 8.29 mmol). The reaction mixture was heated at reflux for 16 h. The reaction mixture was cooled down to ambient temperature. Additional oxalyl dichloride (3.6 mL, 41.5 mmol) was added dropwise. The reaction mixture was stirred at reflux for 4 h then at 25° C. for 48 h. The reaction mixture was concentrated under reduced pressure. The residue was solubilized in EtOAc. The organic layer was washed with a saturated aqueous solution of NaHCO₃then brine, dried over Na₂SO₄, filtered, and concentrated under reduced pressure to afford 4-bromo-2,5-dimethyl-benzoate as an off-white solid (8.03 g, 98% p., 78% y.). m/z [M+H]⁺=no ionization, ¹H NMR (400 MHz, DMSO-d₆): δ (ppm) 7.76 (s, 1H), 7.58 (s, 1H), 3.81 (s, 3H), 2.45 (s, 3H), 2.33 (s, 3H).

### Intermediate BB-04B: methyl 4-bromo-2-(bromomethyl)-5-methyl-benzoate

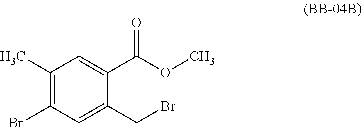

To a stirred solution of 1-bromopyrrolidine-2,5-dione (6.34 g, 35.6 mmol) in acetonitrile (104 mL) were added methyl 4-bromo-2,5-dimethylbenzoate (8.03 g, 32.4 mmol) and AIBN (532 mg, 3.24 mmol). The reaction mixture was irradiated with a white lamp (6200 K) for 3 h. The mixture was concentrated under reduced pressure. The crude was purified by flash chromatography on silica gel using a gradient of EtOAc in cyclohexane from 0% to 20% to afford methyl 4-bromo-2-(bromomethyl)-5-methyl-benzoate as a white solid (9.06 g., 50% p., 44% y.). m/z [M+H]=no ionization.

### Intermediate BB-04C: tert-butyl(4S)-5-amino-4-(5-bromo-6-methyl-1-oxo-isoindolin-2-yl)-5-oxo-pentanoate

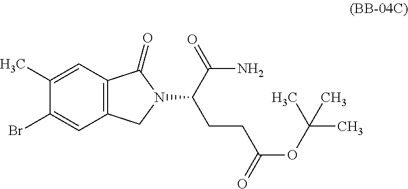 [SMILES: Cc1cc2c(cc1Br)CN([C@@H](CCC(=O)OC(C)(C)C)C(N)=O)C2=O]

To a stirred solution of methyl 4-bromo-2-(bromomethyl)-5-methyl-benzoate (9.06 g, 28.1 mmol) in acetonitrile (90 mL) at ambient temperature were added tert-butyl(4S)-4-amino-4-carbamoylbutanoate hydrochloride (7.39 g, 31.0 mmol) and DIPEA (15 mL, 84.4 mmol). The reaction mixture was stirred at 80° C. for 16 h. The reaction was quenched with the addition of a saturated aqueous solution of NaHCO₃ and EtOAc was added. The aqueous layer was extracted with EtOAc. The combined organic layers were washed with brine, dried over Na₂SO₄, filtered, and concentrated under reduced pressure. The crude material was purified by flash chromatography on silica gel using a gradient of EtOAc/EtOH (3/1) in cyclohexane from 5% to 50% to afford tert-butyl(4S)-5-amino-4-(5-bromo-6-methyl-1-oxo-isoindolin-2-yl)-5-oxo-pentanoate as a white solid (1.70 g, 86% p., 13% y.). m/z [M+Na]⁺=433.1, ¹H NMR (500 MHz, DMSO-d₆): δ (ppm) 7.89 (s, 1H), 7.69 (s, 1H), 7.57 (s, 1H), 7.19 (s, 1H), 4.72 (dd, J=10.2, 4.5 Hz, 1H), 4.57 (d, J=17.6 Hz, 1H), 4.42 (d, J=17.7 Hz, 1H), 2.44 (s, 3H), 2.19-2.09 (m, 3H), 2.00-1.92 (m, 1H), 1.34 (s, 9H).

### Intermediate BB-04

In an autoclave, to a stirred solution of tert-butyl(4S)-5-amino-4-(5-bromo-6-methyl-1-oxo-isoindolin-2-yl)-5-oxo-pentanoate (1.70 g, 4.13 mmol) in DMF (14 mL) were added ethanedioic acid dihydrate (782 mg, 6.20 mmol), XantPhos (120 mg, 0.207 mmol), and Pd(OAc)₂(47.8 mg, 0.207 mmol). The mixture was degassed with argon for 5 min. Next, acetic anhydride (573 μL, 6.20 mmol) and DIPEA (1.1 mL, 6.20 mmol) were added. The mixture was stirred at 100° C. for 16 h. The reaction mixture was cooled to ambient temperature. The reaction mixture was poured in a saturated aqueous solution of NaHCO₃. The aqueous layer was washed with EtOAc. The aqueous layer was acidified to pH 1 with a 12 M aqueous solution of HCl. The aqueous layer was extracted with EtOAc (2×). The combined organic layers were washed with brine, dried over Na₂SO₄, filtered, and dried under reduced pressure. The crude material was purified by reverse-phase preparative chromatography using a gradient of acetonitrile in water (0.1% AcOH) from 0% to 100% then, by chiral supercritical chromatography (Waters Prep SFC200, Chiralpak IC (5 μm, 250×30 mm), CO₂/(MeOH+0.5% IprNH₂) (70/30) as mobile phase, flow rate 100 mL/min, 100 bars) to afford (first isomer eluted)₂-[(1S)-4-tert-butoxy-1-carbamoyl-4-oxo-butyl]-6-methyl-1-oxo-isoindoline-5-carboxylic acid as a white powder (230 mg, 98% p., 15% y.). m/z [M+Na]⁺=399.3, ¹H NMR (400 MHz, DMSO-d₆): δ (ppm) 13.13 (s, 1H), 8.00 (s, 1H), 7.62 (t, J=0.7 Hz, 1H), 7.57 (s, 1H), 7.19 (s, 1H), 4.74 (dd, J=10.3, 4.2 Hz, 1H), 4.60 (d, J=17.6 Hz, 1H), 4.46 (d, J=17.6 Hz, 1H), 2.59 (s, 3H), 2.15 (h, J=2.8 Hz, 3H), 2.06-1.89 (m, 1H), 1.33 (s, 9H).

### INTERMEDIATE BB-05

### 2-[(1R)-4-tert-butoxy-1-carbamoyl-4-oxo-butyl]-6-methyl-1-oxo-isoindoline-5-carboxylic

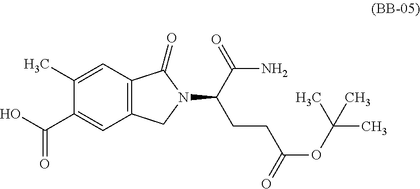 [SMILES: Cc1cc2c(cc1C(=O)O)CN([C@H](CCC(=O)OC(C)(C)C)C(N)=O)C2=O]

In an autoclave, to a stirred solution of tert-butyl(4S)-5-amino-4-(5-bromo-6-methyl-1-oxo-isoindolin-2-yl)-5-oxo-pentanoate (1.70 g, 4.13 mmol) in DMF (14 mL) were added ethanedioic acid dihydrate (782 mg, 6.20 mmol), XantPhos (120 mg, 0.207 mmol), and Pd(OAc)₂(47.8 mg, 207 μmol). The mixture was degassed with argon for 5 min. Next, acetic anhydride (573 μL, 6.20 mmol) and DIPEA (1.1 mL, 6.20 mmol) were added. The mixture was stirred at 100° C. for 16 h. The reaction mixture was cooled to ambient temperature. The reaction mixture was poured in a saturated aqueous solution of NaHCO₃. The aqueous layer was washed with EtOAc. The aqueous layer was acidified to pH 1 with a 12 M aqueous solution of HCl. The aqueous layer was extracted with EtOAc (2×). The combined organic layers were washed with brine, dried over Na₂SO₄, filtered, and dried under reduced pressure. The crude material was purified by reverse-phase preparative chromatography using a gradient of acetonitrile in water (0.1% AcOH) from 0% to 100% then, by chiral supercritical chromatography (Waters Prep SFC200, Chiralpak IC (5 μm, 250×30 mm), CO₂/(MeOH+0.5% iPrNH₂) (70/30) as mobile phase, flow rate 100 mL/min, 100 bars) to afford (second isomer eluted)₂-[(1R)-4-tert-butoxy-1-carbamoyl-4-oxo-butyl]-6-methyl-1-oxo-isoindoline-5-carboxylic acid as a white powder (230 mg, 98% p., 15% y.). m/z [M+Na]⁺=399.3, ¹H NMR (400 MHz, DMSO-d₆): δ (ppm) 13.13 (s, 1H), 7.99 (s, 1H), 7.62 (s, 1H), 7.57 (s, 1H), 7.19 (s, 1H), 4.74 (dd, J=10.3, 4.2 Hz, 1H), 4.59 (d, J=17.6 Hz, 1H), 4.46 (d, J=17.6 Hz, 1H), 2.58 (s, 3H), 2.20-2.10 (m, 3H), 2.02-1.92 (m, 1H), 1.33 (s, 9H).

### INTERMEDIATE BB-06

### 2-[(1S)-4-tert-butoxy-1-carbamoyl-4-oxo-butyl]-1-oxo-6-vinyl-isoindoline-5-carboxylic acid

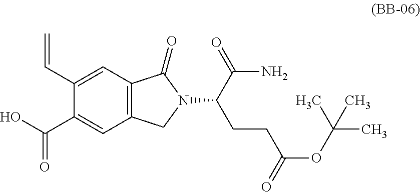 [SMILES: C=Cc1cc2c(cc1C(=O)O)CN([C@@H](CCC(=O)OC(C)(C)C)C(N)=O)C2=O]

### Intermediate BB-06A: methyl 4-bromo-5-iodo-2-methyl-benzoate

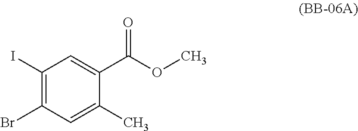

In a three-neck round-bottom flask equipped with a thermometer, under argon, a solution of 4-bromo-5-iodo-2-methylbenzoic acid (5.00 g, 13.9 mmol) in dry MeOH (100 mL) was stirred at 0° C. Thionyl chloride (4.1 mL, 55.7 mmol) was added dropwise over 2 min. The mixture was stirred at ambient temperature for 16 h and then at 60° C. for 4 h. The mixture was concentrated, and the residue was partitioned between a saturated aqueous solution of NaHCO₃ and EtOAc. The aqueous layer was extracted with EtOAc (2×). The combined organic layers were washed with brine, filtered through hydrophobic paper, and concentrated to afford methyl 4-bromo-5-iodo-2-methyl-benzoate as a beige solid (4.77 g, 96% p., 93% y.). m/z [M+H]⁺=no ionization, ¹H NMR (400 MHz, DMSO-d₆): δ (ppm) 8.23 (s, 1H), 7.74 (s, 1H), 3.82 (s, 3H), 2.44 (s, 3H).

### Intermediate BB-06B: methyl 4-bromo-2-(bromomethyl)-5-iodo-benzoate

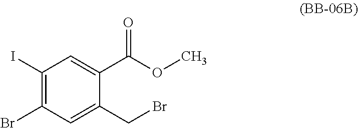

In a sealed tube under argon, AIBN (212 mg, 1.29 mmol) was added to a stirred solution of methyl 4-bromo-5-iodo-2-methyl-benzoate (4.77 g, 12.9 mmol) and 1-bromopyrrolidine-2,5-dione (2.53 g, 14.2 mmol) in dry acetonitrile (50 mL). The mixture was irradiated with a white lamp (6200 K) for 2 h and was concentrated. The crude was purified by flash chromatography on silica gel using a gradient of EtOAc in n-heptane from 0% to 10% to afford methyl 4-bromo-2-(bromomethyl)-5-iodo-benzoate as an off-white solid (5.35 g, 75% p., 72% y.). m/z [M+H]⁺=no ionization.

### Intermediate BB-06C: (tert-butyl(4S)-5-amino-4-(5-bromo-6-iodo-1-oxo-isoindolin-2-yl)-5-oxo-pentanoate

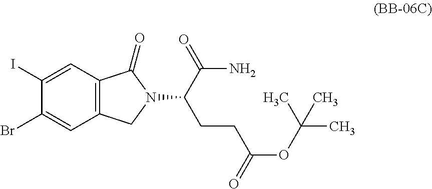 [SMILES: CC(C)(C)OC(=O)CC[C@@H](C(N)=O)N1Cc2cc(Br)c(I)cc2C1=O]

To a stirred suspension of tert-butyl(4S)-4-amino-4-carbamoylbutanoate hydrochloride (3.12 g, 13.1 mmol) in acetonitrile (20 mL) at 0° C. was added DIPEA (4.3 mL, 24.7 mmol) dropwise. The reaction mixture was stirred for 30 min at 0° C. A solution of methyl 4-bromo-2-(bromomethyl)-5-iodo-benzoate (5.35 g, 12.3 mmol) in acetonitrile (35 mL) was added dropwise over 30 min at 0° C. The reaction mixture was then stirred at 70° C. overnight. The mixture was cooled to ambient temperature then concentrated under reduced pressure. The residue was triturated in water (60 mL) for 1 h, filtered, washed with water, and dried under reduced pressure. The residue was triturated in n-pentane/EtOAc (5/1) for 1 h, filtered, washed with n-pentane and dried under reduced pressure at 50° C. overnight to afford tert-butyl(4S)-5-amino-4-(5-bromo-6-iodo-1-oxo-isoindolin-2-yl)-5-oxo-pentanoate as an off-white solid (5.07 g, 92% p., 72% y.). m/z [M+H]⁺=523.2, ¹H NMR (400 MHz, DMSO-d₆): δ (ppm) 8.15 (s, 1H), 8.05 (s, 1H), 7.58 (s, 1H), 7.27-7.15 (m, 1H), 4.77-4.64 (m, 1H), 4.53 (d, J=18.0 Hz, 1H), 4.40 (d, J=18.1 Hz, 1H), 2.23-2.05 (m, 3H), 2.05-1.86 (m, 1H), 1.33 (s, 9H).

### Intermediate BB-06D: tert-butyl(4S)-5-amino-4-(5-bromo-1-oxo-6-vinyl-isoindolin-2-yl)-5-oxo-pentanoate

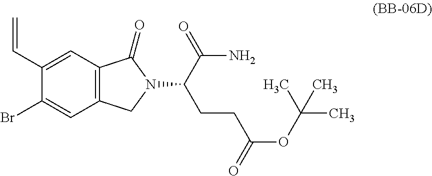 [SMILES: C=Cc1cc2c(cc1Br)CN([C@@H](CCC(=O)OC(C)(C)C)C(N)=O)C2=O]

To a stirred solution of tert-butyl(4S)-5-amino-4-(5-bromo-6-iodo-1-oxo-isoindolin-2-yl)-5-oxo-pentanoate (2.00 g, 3.82 mmol) in a mixture of 1,4-dioxane (15 mL) and water (3.8 mL) were added successively K₂CO₃ (1,32 g, 9.56 mmol) then 4,4,5,5-tetramethyl-2-vinyl-1,3,2-dioxaborolane (778 μL, 4.59 mmol). The reaction mixture was degassed with argon. Pd(PPh₃)₄ (442 mg, 0.382 mmol) was added and the reaction mixture was stirred at 90° C. overnight. Additional 4,4,5,5-tetramethyl-2-vinyl-1,3,2-dioxaborolane (707 mg, 4.59 mmol) and Pd(PPh₃)₄ (442 mg, 0.382 mmol) were added and the reaction mixture was stirred at 90° C. for 24 h. The reaction mixture was cooled to ambient temperature and diluted with water. The aqueous layer was extracted with EtOAc (3×). The combined organic layers were washed with brine, dried over Na₂SO₄, filtered, and concentrated under reduced pressure. The crude was purified by flash chromatography on silica gel using a gradient of EtOAc/EtOH (3/1) in cyclohexane from 1% to 100% to afford tert-butyl(4S)-5-amino-4-(5-bromo-1-oxo-6-vinyl-isoindolin-2-yl)-5-oxo-pentanoate as a yellow solid (2.13 g, 65% p., 85% y.). m/z [M+H]⁺=423.2.

### Intermediate BB-06

In an autoclave, to a stirred solution of tert-butyl(4S)-5-amino-4-(5-bromo-1-oxo-6-vinyl-isoindolin-2-yl)-5-oxo-pentanoate (2.13 g, 5.03 mmol) in DMF (10 mL) were added successively ethanedioic acid dihydrate (950 mg, 7.54 mmol), XantPhos (145 mg, 0.251 mmol), and Pd(OAc)₂(56.9 mg, 0.251 mmol). The reaction mixture was degassed with argon for 5 min. Next, acetic anhydride (697 μL, 7.54 mmol) and DIPEA (1.3 mL, 7.54 mmol) were added. The reaction mixture was stirred at 100° C. overnight. The reaction mixture was cooled to ambient temperature. The reaction mixture was poured into a 1 M aqueous solution of NaHCO₃. The aqueous layer was washed with EtOAc (3×). The aqueous layer was acidified to pH 3-4 with a 12 M aqueous solution of HCl then extracted with EtOAc (3×). The combined organic layers were dried using a phase separator and concentrated under reduced pressure to afford 2-[(1S)-4-tert-butoxy-1-carbamoyl-4-oxo-butyl]-1-oxo-6-vinyl-isoindoline-5-carboxylic acid as an orange oil (1.06 g, 89% p., 48% y.). m/z [M+H]⁺=389.3.

### INTERMEDIATE BB-07

### benzyl 4-benzoylpiperidine-1-carboxylate

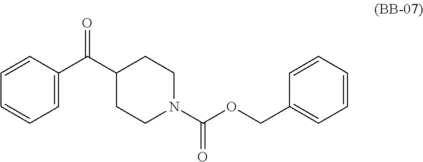 [SMILES: O=C(c1ccccc1)C1CCN(C(=O)OCc2ccccc2)CC1]

To a stirred suspension of phenyl(4-piperidyl)methanone hydrochloride (25.0 g, 0.109 mol) in DCM (155 mL) at 0° C. were added successively triethylamine (35 mL, 0.250 mol) and benzyl carbonochloridate (17 mL, 0.119 mol) dropwise. The reaction mixture was stirred at ambient temperature overnight. The reaction was quenched with the addition of saturated aqueous solution of NH₄Cl. The aqueous layer was extracted with DCM (2×). The combined organic layers were washed with brine, dried using a phase separator, and concentrated under reduced pressure. The crude was purified by flash chromatography on silica gel using a gradient of EtOAc in cyclohexane from 5% to 60%. The fractions were combined and concentrated under reduced pressure to afford benzyl 4-benzoylpiperidine-1-carboxylate as a pale yellow oil (32.5 g, 81% p., 75% y.). m/z [M+H]⁺=324.3, ¹H NMR (400 MHz, DMSO-d₆): δ (ppm) 8.03-7.92 (m, 2H), 7.64 (qt, J=8.6, 1.3 Hz, 1H), 7.58-7.48 (m, 2H), 7.42-7.20 (m, 5H), 5.09 (s, 2H), 4.05 (dt, J=13.1, 3.0 Hz, 2H), 3.67 (tt, J=11.3, 3.6 Hz, 1H), 3.02 (s, 2H), 1.80 (d, J=11.4 Hz, 2H), 1.44 (qd, J=12.2, 4.2 Hz, 2H).

### INTERMEDIATE BB-08

### benzyl 4-[(R)-(2-chloro-3-hydroxy-phenyl)-phenyl-methyl]piperidine-1-carboxylate

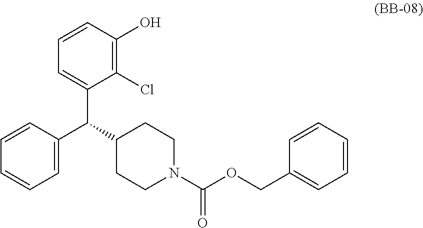 [SMILES: O=C(OCc1ccccc1)N1CCC([C@H](c2ccccc2)c2cccc(O)c2Cl)CC1]

### Intermediate BB-08A: (3-bromo-2-chloro-phenoxy)-tert-butyl-dimethyl-silane

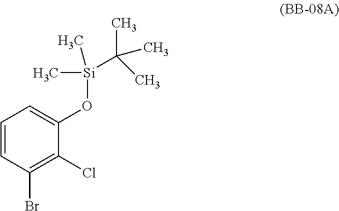 [SMILES: CC(C)(C)[Si](C)(C)Oc1cccc(Br)c1Cl]

To a stirred solution of 3-bromo-2-chlorophenol (16.2 g, 78.1 mmol) in DCM (200 mL) were added successively triethylamine (24 mL, 172 mmol), DMAP (954 mg, 7.81 mmol), and tert-butyl-chloro-dimethyl-silane (12.9 g, 85.9 mmol). The reaction mixture was stirred at ambient temperature overnight. The reaction mixture was filtered and the solid was washed with EtOAc. The filtrate was washed with a saturated aqueous solution of NaHCO₃. The aqueous layer was extracted with EtOAc (2×). The combined organic layers were washed with brine, dried over Na₂SO₄, filtered, and concentrated under reduced pressure. The crude was purified through a pad of silica gel using a mixture of cyclohexane/EtOAc (9/1) as eluent to afford (3-bromo-2-chloro-phenoxy)-tert-butyl-dimethyl-silane as a light-yellow liquid (24.5 g, 92% p., 90% y.). m/z [M+H]⁺=no ionization, ¹H NMR (400 MHz, DMSO-d₆): δ (ppm) 7.36 (dd, J=8.1, 1.4 Hz, 1H), 7.18 (t, J=8.2 Hz, 1H), 7.03 (dd, J=8.2, 1.4 Hz, 1H), 0.99 (s, 9H), 0.23 (s, 6H).

### Intermediate BB-08B: benzyl 4-[[3-[tert-butyl(dimethyl) silyl]oxy-2-chloro-phenyl]-phenyl-methyl]piperidine-1-carboxylate

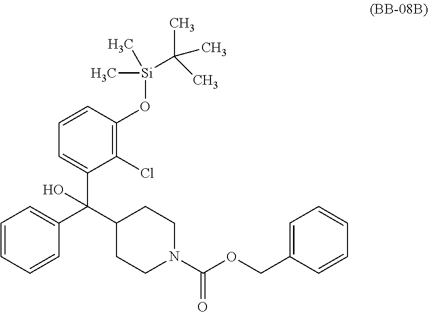 [SMILES: CC(C)(C)[Si](C)(C)Oc1cccc(C(O)(c2ccccc2)C2CCN(C(=O)OCc3ccccc3)CC2)c1Cl]

To a stirred solution of (3-bromo-2-chloro-phenoxy)-tert-butyl-dimethyl-silane (16.7 g, 47.8 mmol) and benzyl 4-benzoylpiperidine-1-carboxylate (14.0 g, 43.4 mmol) in dry THF (215 mL) at −78° C. under nitrogen was added 1.6 M n-butyllithium in hexanes (41 mL, 65.1 mmol). The reaction mixture was stirred at −78° C. for 10 min and at ambient temperature for 16 h. The reaction was quenched with the addition of a saturated aqueous solution of NH₄Cl, water. EtOAc were added. The phases were separated, and the aqueous phase was extracted with EtOAc. The combined organic layers were washed with brine, dried using a phase separator and concentrated under vacuum. The crude material was purified on a pad of silica gel using cyclohexane/EtOAc (80/20) as eluent to afford benzyl 4-[[3-[tert-butyl(dimethyl) silyl]oxy-2-chloro-phenyl]-hydroxy-phenyl-methyl]piperidine-1-carboxylate as a yellow oil (18.5 g, 83% p., 63% y.). m/z [M+H]⁺=no ionization.

### Intermediate BB-08C: benzyl 4-[[3-[tert-butyl(dimethyl) silyl]oxy-2-chloro-phenyl]-phenyl-methyl]piperidine-1-carboxylate

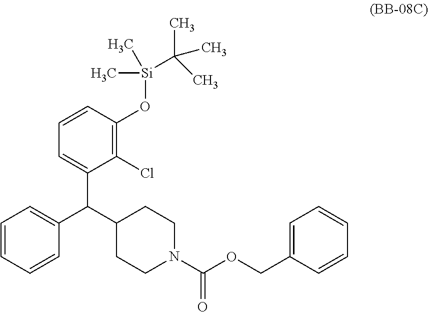 [SMILES: CC(C)(C)[Si](C)(C)Oc1cccc(C(c2ccccc2)C2CCN(C(=O)OCc3ccccc3)CC2)c1Cl]

To a stirred solution of triethylsilane (13 mL, 81.4 mmol) and TFA (62 mL, 814 mmol) in DCM (250 mL) at −20° C. was added a solution of benzyl 4-[[3-[tert-butyl (dimethyl) silyl]oxy-2-chloro-phenyl]-hydroxy-phenyl-methyl]piperidine-1-carboxylate (18.5 g, 27.1 mmol) in DCM (80 mL) dropwise over 1 h. The mixture was warmed up to ambient temperature and stirred overnight. The reaction mixture was slowly poured into a 2 M aqueous solution of NaOH (407 mL, 814 mmol) precooled at 0° C. The layers were separated, and the aqueous layer extracted with DCM (2×). The combined organic layers were washed with brine, dried over Na₂SO₄, filtered, and concentrated under reduced pressure. The crude was purified through a pad of silica gel using cyclohexane/EtOAc (85/15) as eluent to afford benzyl 4-[[3-[tert-butyl(dimethyl) silyl]oxy-2-chloro-phenyl]-phenyl-methyl]piperidine-1-carboxylate as a colorless oil (10.8 g, 98% p., 71% y.). m/z [M+H]⁺=550.5, ¹H NMR (400 MHz, DMSO-d₆): δ (ppm) 7.39-7.32 (m, 6H), 7.31-7.24 (m, 4H), 7.23-7.11 (m, 2H), 6.81 (dd, J=7.9, 1.5 Hz, 1H), 5.05 (s, 2H), 4.14 (d, J=11.2 Hz, 1H), 3.96 (dd, J=13.4, 3.2 Hz, 2H), 2.77 (s, 2H), 2.48-2.36 (m, 1H), 1.51-1.41 (m, 1H), 1.36 (m, 1H), 1.07-0.94 (m, 11H), 0.19 (s, 3H), 0.16 (s, 3H).

### Intermediate BB-08

To a stirred solution of benzyl 4-[[3-[tert-butyl(dimethyl) silyl]oxy-2-chloro-phenyl]-phenyl-methyl]piperidine-1-carboxylate (10.8 g, 19.3 mmol) in THF (60 mL) under nitrogen was added 1 M TBAF in THF (29 mL, 28.9 mmol) dropwise. The reaction mixture was stirred at ambient temperature for 1 h. The reaction was quenched with the addition of water. The aqueous layer was extracted with EtOAc (3×). The combined organic layers were washed with brine, dried over Na₂SO₄, filtered, and concentrated under reduced pressure. The crude was purified by flash chromatography on silica gel using a gradient of EtOAc in cyclohexane from 1% to 50% then, by chiral supercritical chromatography (Supersep, Chiralpak AD-H (20 μm, 250×50 mm), CO₂/MeOH 65/35 as mobile phase, flow rate 900 g/min, 100 bars) and by chiral reverse-phase (Novasep, Chiralpack OD-H (20 μm, 300× 76.6 mm), MeOH as mobile phase, flow rate 275 mL/min) to afford benzyl 4-[(R)-(2-chloro-3-hydroxy-phenyl)-phenyl-methyl]piperidine-1-carboxylate (first isomer eluted) as a white solid (1.88 g, 99% p, 22% y., e. e.>99.9%). m/z [M+H]⁺=436.3, ¹H NMR (400 MHz, DMSO-d₆): δ (ppm) 10.01 (s, 1H), 7.39-7.29 (m, 7H), 7.26 (t, J=7.6 Hz, 2H), 7.20-7.07 (m, 3H), 6.80-6.74 (m, 1H), 5.05 (s, 2H), 4.12 (d, J=11.2 Hz, 1H), 3.96 (dt, J=13.2, 3.4 Hz, 2H), 2.78 (s, 2H), 2.42 (m, 1H), 1.50 (d, J=13.0 Hz, 1H), 1.36 (d, J=13.2 Hz, 1H), 1.09-0.89 (m, 2H), [α]_{D}=+45°mL·dm⁻¹ g⁻¹[20° C., Na lamp, 589 nm, C=2.8 mg/mL, EtOH].

### INTERMEDIATE BB-09

### benzyl 4-[(R)-(2-chloro-3-hydroxy-phenyl)-phenyl-methyl]piperidine-1-carboxylate

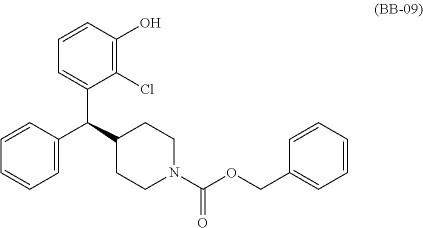 [SMILES: O=C(OCc1ccccc1)N1CCC([C@@H](c2ccccc2)c2cccc(O)c2Cl)CC1]

To a stirred solution of benzyl 4-[[3-[tert-butyl(dimethyl) silyl]oxy-2-chloro-phenyl]-phenyl-methyl]piperidine-1-carboxylate (10.8 g, 19.3 mmol) in THF (60 mL) under nitrogen was added 1 M TBAF in THF (29 mL, 28.9 mmol) dropwise. The reaction mixture was stirred at ambient temperature for 1 h. The reaction was quenched with the addition of water. The aqueous layer was extracted with EtOAc (3×). The combined organic layers were washed with brine, dried over Na₂SO₄, filtered, and concentrated under reduced pressure. The crude was purified by flash chromatography on silica gel using a gradient of EtOAc in cyclohexane from 1% to 50% then, by chiral supercritical chromatography (Supersep, Chiralpak AD-H (20 μm, 250×50 mm), CO₂/MeOH 65/35 as mobile phase, flow rate 900 g/min, 100 bars) and by chiral reverse-phase (Novasep, Chiralpack OD-H (20 μm, 300×76.6 mm), MeOH as mobile phase, flow rate 275 mL/min) to afford benzyl 4-[(R)-(2-chloro-3-hydroxy-phenyl)-phenyl-methyl]piperidine-1-carboxylate (second isomer eluted) as a white solid (2.05 g, 95% p, 24% y., e.e.>99.9%). m/z [M+H]⁺=436.3, ¹H NMR (400 MHz, DMSO-d₆): δ (ppm) 10.00 (s, 1H), 7.40-7.22 (m, 9H), 7.19-7.05 (m, 3H), 6.82-6.74 (m, 1H), 5.05 (s, 2H), 4.12 (d, J=11.3 Hz, 1H), 3.96 (d, J=13.2 Hz, 2H), 2.77 (s, 2H), 2.42 (q, J=11.7 Hz, 1H), 1.50 (d, J=13.0 Hz, 1H), 1.36 (d, J=13.1 Hz, 1H), 1.11-0.89 (m, 2H), [α]_{D}=−46° mL·dm⁻¹ g⁻¹[20° C., Na lamp, 589 nm, C=2.8 mg/mL, EtOH].

### INTERMEDIATE BB-10

### 4-[(2-chlorophenyl)-phenyl-methyl]piperidine hydroiodide

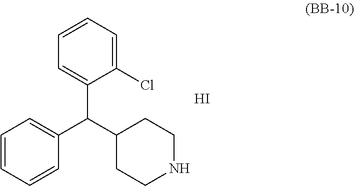

### Intermediate BB-10A: benzyl 4-[(2-chlorophenyl)-hydroxy-phenyl-methyl]piperidine-1-carboxylate

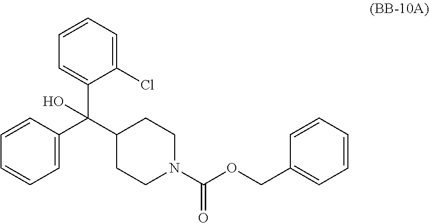 [SMILES: O=C(OCc1ccccc1)N1CCC(C(O)(c2ccccc2)c2ccccc2Cl)CC1]

To a stirred solution of 1-bromo-2-chloro-benzene (548 μL, 4.64 mmol) in dry THF (15 mL) at −78° C. under argon was added a 2.5 M solution of n-butyllithium in hexanes (1.9 mL, 4.64 mmol) over 10 min. After the end of addition, a solution of benzyl 4-benzoylpiperidine-1-carboxylate (1.00 g, 3.09 mmol) in dry THF (7.5 mL) was added over 15 minutes keeping the temperature below −60° C. The reaction mixture was allowed to warm up to ambient temperature overnight. The reaction was quenched with the addition of saturated aqueous solution of NH₄Cl. Water and EtOAc were added. The phases were separated, and the aqueous phase extracted with EtOAc. The combined organic layers were washed with brine, dried over hydrophobic paper, and concentrated under reduced pressure. The resulting white foam was triturated in Et₂O/n-pentane (9/1) (5.0 mL). The resulting precipitate was filtered, washed with n-pentane, and dried under reduced pressure at 50° C. overnight to afford benzyl 4-[(2-chlorophenyl)-hydroxy-phenyl-methyl]piperidine-1-carboxylate as a white powder (958 mg, 95% p., 68% y). m/z [M+H]⁺=436.2, ¹H NMR (400 MHz, DMSO-d₆): δ (ppm) 8.05-7.98 (m, 1H), 7.45-7.10 (m, 13H), 5.50 (s, 1H), 5.05 (s, 2H), 4.04 (d, J=13.2 Hz, 2H), 3.16 (s, 1H), 2.88 (s, 2H), 1.54 (s, 1H), 1.33 (dddd, J=43.8, 19.5, 14.1, 5.8 Hz, 3H).

### Intermediate BB-10B: benzyl 4-[(2-chlorophenyl)-phenyl-methyl]piperidine-1-carboxylate

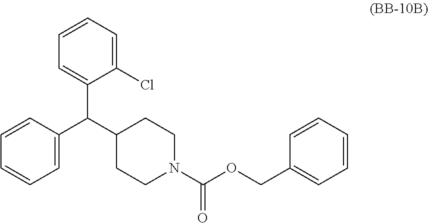 [SMILES: O=C(OCc1ccccc1)N1CCC(C(c2ccccc2)c2ccccc2Cl)CC1]

To a stirred solution of benzyl 4-[(2-chlorophenyl)-hydroxy-phenyl-methyl]piperidine-1-carboxylate (958 mg, 2.09 mmol) in DCM (7.5 mL) at 0° C. under nitrogen were added successively triethylsilane (1.0 mL, 6.26 mmol) and TFA (4.8 mL, 62.6 mmol) while maintaining the temperature below 5° C. The reaction mixture was stirred at room temperature overnight. The reaction was quenched at 0° C. with the addition of a saturated aqueous solution of K₂CO₃. The phases were separated, and the aqueous phase extracted with DCM. The combined organic layers were washed with brine, dried over hydrophobic paper, filtered, and concentrated under vacuum. The crude material was purified by flash chromatography on silica gel using a gradient of EtOAc in n-heptane from 0% to 15%. The fractions were concentrated under vacuum to afford benzyl 4-[(2-chlorophenyl)-phenyl-methyl]piperidine-1-carboxylate as a colorless oil (206 mg, 98% p., 23% y.). m/z [M+H]⁺=420.0, ¹H NMR (400 MHz, DMSO-d₆): δ (ppm) 7.70 (dd, J=7.9, 1.6 Hz, 1H), 7.40-7.24 (m, 11H), 7.21-7.13 (m, 2H), 5.05 (s, 2H), 4.11 (d, J=11.2 Hz, 1H), 4.00-3.83 (m, 2H), 2.78 (s, 2H), 2.50-2.42 (m, 1H), 1.46 (d, J=13.3 Hz, 1H), 1.38 (d, J=13.7 Hz, 1H), 1.11-0.95 (m, 2H).

### Intermediate BB-10

A sealed vial was charged with a suspension of benzyl 4-[(2-chlorophenyl)-phenyl-methyl]piperidine-1-carboxylate (130 mg, 0.310 mmol) in dry acetonitrile (3.9 mL) at ambient temperature under nitrogen. Iodo (trimethyl) silane (132 μL, 0.929 mmol) was added dropwise, the mixture was stirred at ambient temperature for 1 h. The mixture was stirred at 0° C. and MeOH (150 μL, 3.71 mmol) was added dropwise. The mixture was stirred at ambient temperature for 20 min and was then concentrated under reduced pressure. Et₂O was added to the residue. The resulting mixture was stirred at ambient temperature for 1 h. The solid was filtered, washed with Et₂O, and dried under reduced pressure at 50° C. for 16 h to afford 4-[(2-chlorophenyl)-phenyl-methyl]piperidine hydroiodide as a white powder (114 mg, 99% p., 88% y.). m/z [M+H]⁺=286.3, ¹H NMR (400 MHz, DMSO-d₆): δ (ppm) 8.41 (s, 1H), 8.09 (s, 1H), 7.66 (dd, J=7.9, 1.7 Hz, 1H), 7.45-7.15 (m, 8H), 4.11 (d, J=11.2 Hz, 1H), 3.24 (d, J=12.6 Hz, 2H), 2.84 (s, 2H), 2.67-2.54 (m, 1H), 1.55 (t, J=16.6 Hz, 2H), 1.34-1.20 (m, 2H).

### INTERMEDIATE BB-11

### 4-[(R)-(2-chlorophenyl)-phenyl-methyl]piperidine hydroiodide

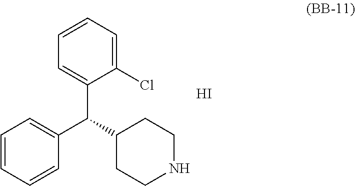

### Intermediate BB-11A: benzyl 4-[(R)-(2-chlorophenyl)-phenyl-methyl]piperidine-1-carboxylate

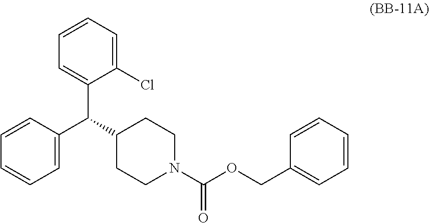 [SMILES: O=C(OCc1ccccc1)N1CCC([C@H](c2ccccc2)c2ccccc2Cl)CC1]

To a stirred solution of benzyl 4-[(2-chlorophenyl)-hydroxy-phenyl-methyl]piperidine-1-carboxylate (2.03 g, 4.42 mmol) in DCM (16 mL) at −50° C. under nitrogen were added triethylsilane (2.14 mL, 13.3 mmol) dropwise. TFA (10 mL, 0.133 mol) was added dropwise very slowly (keeping the temperature below −40° C.). DCM (16 mL) was added. The reaction mixture was allowed to warm up to ambient temperature overnight. The reaction mixture was slowly added to a stirred aqueous solution of 10 M NaOH (13 mL, 0.133 mol) at 0° C. (maintaining the temperature below 10° C.). The phases were separated, and the aqueous phase extracted with EtOAc. The combined organic layers were washed with brine, dried using hydrophobic paper and concentrated under reduced pressure. The crude material was purified by flash chromatography on silica gel using a gradient of EtOAc in n-heptane from 0% to 12%. The desired fractions were concentrated under reduced pressure to afford (benzyl 4-[(2-chlorophenyl)-phenyl-methyl]piperidine-1-carboxylate as a colorless oil (873 mg, 97% p., 46% y.) then, by chiral supercritical chromatography (Waters Prep SFC200, Chiralpak AD (5 μm, 250×30 mm), CO₂/iPrOH (70/30) as mobile phase, flow rate 100 mL/min) to afford benzyl 4-[(R)-(2-chlorophenyl)-phenyl-methyl]piperidine-1-carboxylate (first isomer) as a colorless gum (370 mg, 93% p., 19% y., e. e.>99.9%). m/z [M+H]⁺=420.3, ¹H NMR (400 MHz, DMSO-d₆): δ (ppm) 7.70 (dd, J=1.7, 7.8 Hz, 1H), 7.33 (m, 11H), 7.18 (m, 2H), 5.05 (s, 2H), 4.11 (d, J=11.2 Hz, 1H), 3.97 (d, J=13.2 Hz, 2H), 2.78 (s, 2H), 1.42 (dd, J=13.2, 32.7 Hz, 2H), 1.02 (m, 3H), [α]_{D}=+17° mL·dm⁻¹.g⁻¹[20° C., Na lamp, 589 nm, C=6.2 mg/mL, EtOH].

### Intermediate BB-11

A vial was charged with a suspension of (benzyl 4-(R)-(2-chlorophenyl)-phenyl-methyl]piperidine-1-carboxylate, 370 mg, 0.881 mmol) in dry acetonitrile (8.8 mL) at ambient temperature under nitrogen. Iodo (trimethyl) silane (376 μL, 2.64 mmol) was added dropwise and the mixture was stirred at ambient temperature for 1 h. The mixture was stirred at 0° C. MeOH (428 μL, 10.6 mmol) was added dropwise and the mixture was stirred at ambient temperature for 20 min and was concentrated. Et₂O was added to the residue. The resulting mixture was stirred at ambient temperature for 1 h. The solid was filtered, washed with Et₂O, and dried under reduced pressure at ambient temperature for 16 h to afford 4-[(R)-(2-chlorophenyl)-phenyl-methyl]piperidine hydroiodide as a yellow solid (309 mg, 95% p., 81% y.). m/z [M+H]⁺=286.2, ¹H NMR (400 MHz, DMSO-d₆): δ (ppm) 8.41 (s, 1H), 8.09 (s, 1H), 7.66 (dd, J=1.6, 7.9 Hz, 1H), 7.36 (m, 6H), 7.21 (m, 2H), 4.11 (d, J=11.2 Hz, 1H), 3.24 (d, J=12.5 Hz, 2H), 2.84 (p, J=11.4, 11.4, 11.4, 11.4 Hz, 2H), 2.60 (m, 1H), 1.55 (m, 2H), 1.26 (m, 2H), [α]_{D}=+25° mL·dm⁻¹ g⁻¹[20° C., Na lamp, 589 nm, C=2.75 mg/mL, MeOH].

### INTERMEDIATE BB-12

### 4-[(S)-(2-chlorophenyl)-phenyl-methyl]piperidine hydroiodide

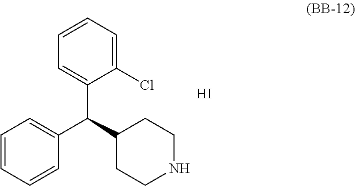

### Intermediate BB-12A: benzyl 4-[(S)-(2-chlorophenyl)-phenyl-methyl]piperidine-1-carboxylate

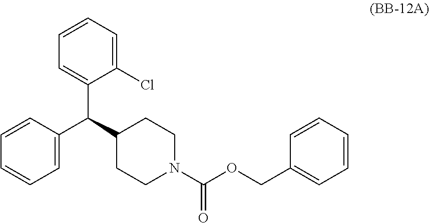 [SMILES: O=C(OCc1ccccc1)N1CCC([C@@H](c2ccccc2)c2ccccc2Cl)CC1]

To a stirred solution of benzyl 4-[(2-chlorophenyl)-hydroxy-phenyl-methyl]piperidine-1-carboxylate (2.03 g, 4.42 mmol) in DCM (16 mL) at −50° C. under nitrogen were added triethylsilane (2.1 mL, 13.3 mmol) dropwise. TFA (10 mL, 0.133 mol) was added dropwise (keeping the temperature below −40° C.). DCM (16 mL) was added. The reaction mixture was allowed to warm up to ambient temperature overnight. The reaction mixture was slowly added into a stirred 10 M aqueous solution of NaOH (13 mL, 133 mmol) at 0° C. (keeping the temperature below 10° C.). The phases were separated and the aqueous phase was extracted with EtOAc. The combined organic layers were washed with brine, dried using hydrophobic paper and concentrated under reduced pressure. The crude material was purified by flash chromatography on silica gel using a gradient of EtOAc in n-heptane from 0% to 12%. The fractions were concentrated under reduced pressure to afford benzyl 4-[(2-chlorophenyl)-phenyl-methyl]piperidine-1-carboxylate as a colorless oil (873 mg, 97% p., 46% y.) then, by chiral supercritical chromatography (Waters Prep SFC200, Chiralpak AD (5 μm, 250×30 mm), CO₂/iPrOH (70/30) as mobile phase, flow rate 100 mL/min) to afford benzyl 4-[(S)-(2-chlorophenyl)-phenyl-methyl]piperidine-1-carboxylate (second isomer) as a colorless gum (380 mg, 97% p., 20% y., e. e.=98.8%). m/z [M+H]⁺=420.3, ¹H NMR (400 MHz, DMSO-d₆): δ (ppm) 7.70 (dd, J=1.7, 7.8 Hz, 1H), 7.33 (m, 11H), 7.18 (m, 2H), 5.05 (s, 2H), 4.11 (d, J=11.2 Hz, 1H), 3.97 (d, J=13.2 Hz, 2H), 2.78 (s, 2H), 1.42 (dd, J=13.2, 32.7 Hz, 2H), 1.02 (m, 3H), [α]_{D}=−24° mL·dm⁻¹ g⁻¹[20° C., Na lamp, 589 nm, C=4.65 mg/mL, EtOH].

### Intermediate BB-12

A vial was charged with a suspension of benzyl 4-[(S)-(2-chlorophenyl)-phenyl-methyl]piperidine-1-carboxylate (380 mg, 0.905 mmol) in dry acetonitrile (9.0 mL) at ambient temperature under nitrogen. Iodo (trimethyl) silane (386 μL, 2.71 mmol) was added dropwise, the mixture was stirred at ambient temperature for 1 h. The mixture was stirred at 0° C. and MeOH (440 μL, 10.9 mmol) was added dropwise. The mixture was stirred at ambient temperature for 20 min and was concentrated. Et₂O was added to the residue. The resulting mixture was stirred at ambient temperature for 1 h. The solid was filtered, washed with Et₂O, and dried under reduced pressure at ambient temperature for 16 h to afford 4-[(S)-(2-chlorophenyl)-phenyl-methyl]piperidine hydroiodide as a yellow solid (324 mg, 96% p., 83% y.). m/z [M+H]⁺=286.2, ¹H NMR (400 MHz, DMSO-d₆): δ (ppm) 8.41 (s, 1H), 8.09 (s, 1H), 7.66 (dd, J=1.6, 7.9 Hz, 1H), 7.36 (m, 6H), 7.21 (m, 2H), 4.11 (d, J=11.2 Hz, 1H), 3.24 (d, J=12.5 Hz, 2H), 2.84 (p, J=11.4, 11.4, 11.4, 11.4 Hz, 2H), 2.60 (m, 1H), 1.55 (m, 2H), 1.26 (m, 2H), [α]_{D}=−24° mL·dm⁻¹ g⁻¹[20° C., Na lamp, 589 nm, C=2.45 mg/mL, MeOH].

### INTERMEDIATE BB-13

### 4-[(R)-(2-chloro-3-methoxy-phenyl)-phenyl-methyl]piperidine hydroiodide

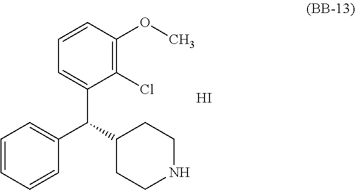

### Intermediate BB-13A: benzyl 4-[(2-chloro-3-methoxy-phenyl)-hydroxy-phenyl-methyl]piperidine-1-carboxylate

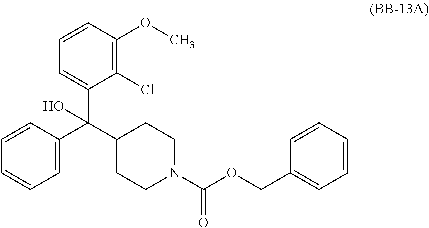 [SMILES: COc1cccc(C(O)(c2ccccc2)C2CCN(C(=O)OCc3ccccc3)CC2)c1Cl]

In a three neck round-bottom flask equipped with a thermometer, a 2.5 M solution of n-butyllithium in hexanes (11 mL, 27.6 mmol) was added to a solution of 1-bromo-2-chloro-3-methoxybenzene (6.23 g, 27.6 mmol) in THF (9.0 mL) and hexanes (81 mL) at −78° C. under argon, over 10 min (keeping the temperature below −70° C.). Directly after the end of the first addition, a solution of benzyl 4-benzoylpiperidine-1-carboxylate (6.00 g, 18.4 mmol) in THF (45 mL) was added over 30 min keeping the temperature below −60° C. The reaction mixture was allowed to warm up to ambient temperature overnight. The reaction was quenched with the addition of a saturated aqueous solution of NH₄Cl. Water and EtOAc were added. The phases were separated and the aqueous phase was extracted with EtOAc. The combined organic layers were washed with brine, dried over Na₂SO₄, filtered, and concentrated under reduced pressure. The crude material was purified by flash chromatography on silica gel using a gradient of EtOAc in cyclohexane from 0% to 100%. The desired fractions were combined and concentrated under reduced pressure. The solid was triturated in cyclohexane and stirred at ambient temperature for 20 min. The precipitate was filtered and dried under reduced pressure at 50° C. for 16 h to afford benzyl 4-[(2-chloro-3-methoxy-phenyl)-hydroxy-phenyl-methyl]piperidine-1-carboxylate as a white powder (710 mg, 89% p., 7% y.). m/z [M+H]⁺=466.3, ¹H NMR (400 MHz, DMSO-d₆): δ (ppm) 7.62 (dd, J=1.4, 8.1 Hz, 1H), 7.33 (m, 8H), 7.24 (dd, J=6.9, 8.3 Hz, 2H), 7.14 (m, 1H), 7.00 (dd, J=1.4, 8.3 Hz, 1H), 5.45 (s, 1H), 5.05 (s, 2H), 4.03 (m, 2H), 3.75 (s, 3H), 3.19 (s, 1H), 2.86 (s, 2H), 1.57 (s, 1H), 1.26 (qt, J=10.5, 10.5, 20.0, 20.0, 20.0 Hz, 3H).

### Intermediate BB-13B: benzyl 4-[(R)-(2-chloro-3-methoxy-phenyl)-phenyl-methyl]piperidine-1-carboxylate

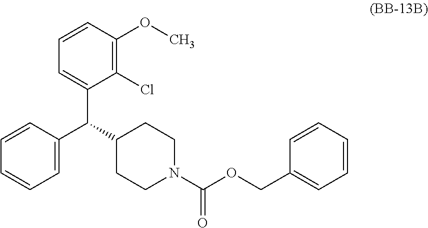 [SMILES: COc1cccc([C@@H](c2ccccc2)C2CCN(C(=O)OCc3ccccc3)CC2)c1Cl]

To a stirred solution of benzyl 4-[(2-chloro-3-methoxy-phenyl)-hydroxy-phenyl-methyl]piperidine-1-carboxylate (5.39 g, 11.6 mmol) in DCM (88 mL) at −50° C. under nitrogen were added triethylsilane (4.80 mL, 29.8 mmol) dropwise. A solution of TFA (23.0 mL, 0.300 mol) in DCM (44 mL) was added dropwise very slowly (keeping the temperature below −40° C.). The reaction mixture was allowed to warm up to ambient temperature and was stirred overnight. The reaction mixture was slowly poured to a stirred aqueous solution of 2 M NaOH (150 mL, 0.300 mol) at 0° C. (keeping the temperature below 10° C.). The phases were separated and the aqueous phase was extracted with EtOAc. The combined organic layers were washed with brine, dried using a hydrophobic paper and concentrated under reduced pressure. The crude material was purified by flash chromatography on silica gel using a gradient of EtOAc in n-heptane from 0% to 20%. The desired fractions were concentrated under reduced pressure to afford a colorless gum (benzyl 4-[(2-chloro-3-methoxy-phenyl)-phenyl-methyl]piperidine-1-carboxylate (4.04 g, 95% p., 73% y)). The product was purified by chiral reverse-phase (Hypersep, Chiralpak IC (20 μm, 300×50 mm), MeOH as mobile phase, flow rate 120 mL/min) to afford benzyl 4-[(R)-(2-chloro-3-methoxy-phenyl)-phenyl-methyl]piperidine-1-carboxylate (first isomer eluted) as a colorless gum (1.68 g, 90% p, 29% y., e.e.>99.9%). m/z [M+H]⁺=450.3, ¹H NMR (400 MHz, DMSO-d₆): δ (ppm) 7.32 (m, 11H), 7.16 (m, 1H), 6.95 (dd, J=2.2, 7.4 Hz, 1H), 5.05 (s, 2H), 4.16 (d, J=11.2 Hz, 1H), 3.96 (d, J=13.2 Hz, 2H), 3.80 (s, 3H), 2.78 (s, 2H), 2.45 (t, J=11.3, 11.3 Hz, 1H), 1.42 (dd, J=13.2, 36.8 Hz, 2H), 1.00 (dd, J=12.9, 25.7 Hz, 2H).

### Intermediate BB-13

A vial was charged with a suspension of benzyl 4-[(R)-(2-chloro-3-methoxy-phenyl)-phenyl-methyl]piperidine-1-carboxylate (1.88 g, 3.73 mmol) in dry acetonitrile (37 mL) at ambient temperature under nitrogen. Iodo (trimethyl) silane (1.6 mL, 11.2 mmol) was added dropwise, the mixture was stirred at ambient temperature for 1 h. The mixture was cooled to 0° C. and MeOH (1.8 mL, 44.8 mmol) was added dropwise. The mixture was stirred at ambient temperature for 20 min and was concentrated under reduced pressure. Et₂O was added to the residue. The resulting mixture was stirred at ambient temperature for 1 h. The solid was filtered, washed with Et₂O, and dried under reduced pressure at ambient temperature for the weekend to afford 4-[(R)-(2-chloro-3-methoxy-phenyl)-phenyl-methyl]piperidine hydroiodide as a yellow solid (1.70 g, 95% p., 97% y.). m/z [M+H]⁺=316.3, ¹H NMR (400 MHz, DMSO-d₆): δ 8.40 (s, 1H), 8.09 (s, 1H), 7.25 (m, 7H), 6.98 (dd, J=1.3, 8.2 Hz, 1H), 4.16 (d, J=11.3 Hz, 1H), 3.81 (s, 3H), 3.22 (s, 2H), 2.84 (m, 2H), 2.58 (d, J=11.3 Hz, 1H), 1.55 (m, 2H), 1.26 (m, 2H), [α]_{D}=+54° mL·dm⁻¹ g⁻¹[20° C., Na lamp, 589 nm, C=2.4 mg/mL, MeOH].

### INTERMEDIATE BB-14

### 4-[(S)-(2-chloro-3-methoxy-phenyl)-phenyl-methyl]piperidine hydroiodide

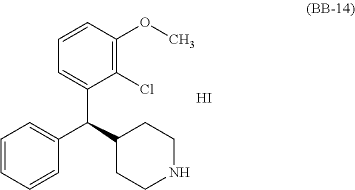

### Intermediate BB-14A: benzyl 4-[(S)-(2-chloro-3-methoxy-phenyl)-phenyl-methyl]piperidine-1-carboxylate

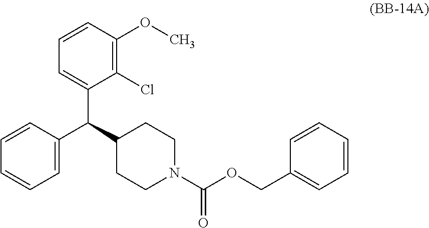 [SMILES: COc1cccc([C@H](c2ccccc2)C2CCN(C(=O)OCc3ccccc3)CC2)c1Cl]

To a stirred solution of benzyl 4-[(2-chloro-3-methoxy-phenyl)-hydroxy-phenyl-methyl]piperidine-1-carboxylate (5.39 g, 11.6 mmol) in DCM (88 mL) at −50° C. under nitrogen were added triethylsilane (4.8 mL, 29.8 mmol) dropwise. A solution of TFA (23 mL, 300 mmol) in DCM (44 mL) was added dropwise (keeping the temperature below −40° C.). The reaction mixture was allowed to warm up to ambient temperature and was stirred overnight. The reaction mixture was slowly poured to a stirred 2 M aqueous solution of NaOH (150 mL, 300 mmol) at 0° C. (keeping the temperature below 10° C.). The phases were separated, and the aqueous phase extracted with EtOAc. The combined organic layers were washed with brine, dried using hydrophobic paper and concentrated under reduced pressure. The crude material was purified by flash chromatography on silica gel using a gradient of EtOAc in n-heptane from 0% to 20%. Fractions were concentrated under reduced pressure to afford benzyl 4-[(2-chloro-3-methoxy-phenyl)-phenyl-methyl]piperidine-1-carboxylate as a colorless gum (4.04 g, 95% p., 73% y). The product was purified by chiral reverse-phase (Hypersep, Chiralpak IC (20 μm, 300× 50 mm), MeOH as mobile phase, flow rate 120 mL/min) to afford benzyl 4-[(S)-(2-chloro-3-methoxy-phenyl)-phenyl-methyl]piperidine-1-carboxylate (second isomer eluted) as a colorless gum (1.78 g, 99% p, 34% y., e. e.=99.5%). m/z [M+H]⁺=450.3, ¹H NMR (400 MHz, DMSO-d₆): δ (ppm) 7.31 (m, 11H), 7.16 (m, 1H), 6.95 (dd, J=2.2, 7.4 Hz, 1H), 5.05 (s, 2H), 4.16 (d, J=11.2 Hz, 1H), 3.96 (m, 2H), 3.80 (s, 3H), 2.78 (s, 2H), 2.45 (m, 1H), 1.42 (dd, J=13.1, 36.6 Hz, 2H), 1.01 (m, 2H).

### Intermediate BB-14

A vial was charged with a suspension of benzyl 4-[(S)-(2-chloro-3-methoxy-phenyl)-phenyl-methyl]piperidine-1-carboxylate (1.93 g, 4.24 mmol) in dry acetonitrile (39 mL) at ambient temperature under nitrogen. Iodo (trimethyl) silane (1.8 mL, 12.7 mmol) was added dropwise. The mixture was stirred at ambient temperature for 1 h. The mixture was cooled to 0° C. and MeOH (2.1 mL, 50.9 mmol) was added dropwise. The mixture was stirred at ambient temperature for 20 min and was concentrated. Et₂O was added to the residue. The resulting mixture was stirred at ambient temperature for 1 h. The solid was filtered, washed with Et₂O, and dried under reduced pressure at ambient temperature for 72 h to afford 4-[(S)-(2-chloro-3-methoxy-phenyl)-phenyl-methyl]piperidine hydroiodide as a yellow solid (1.74 g, 97% p., 90% y). m/z [M+H]⁺=316.3, ¹H NMR (400 MHz, DMSO-d₆): δ 8.40 (s, 1H), 8.09 (s, 1H), 7.26 (m, 7H), 6.98 (dd, J=1.2, 8.2 Hz, 1H), 4.16 (d, J=11.3 Hz, 1H), 3.81 (s, 3H), 3.22 (s, 2H), 2.84 (m, 2H), 2.58 (q, J=11.5, 11.5, 11.6 Hz, 1H), 1.55 (m, 2H), 1.27 (m, 2H), [α]_{D}=−53° mL·dm⁻¹ g⁻¹[20° C., Na lamp, 589 nm, C=3.9 mg/mL, MeOH].

### INTERMEDIATE BB-15

### 6-[(1S)-4-tert-butoxy-1-carbamoyl-4-oxo-butyl]-2-methyl-7-oxo-5H-pyrrolo[3,4-b]pyrazine-3-carboxylic acid

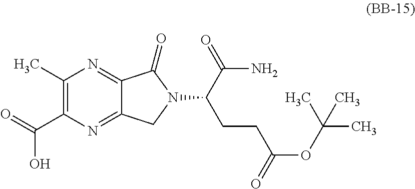 [SMILES: Cc1nc2c(nc1C(=O)O)CN([C@@H](CCC(=O)OC(C)(C)C)C(N)=O)C2=O]

### Intermediate BB-15A: diethyl 3-(bromomethyl)-6-methyl-pyrazine-2,5-dicarboxylate

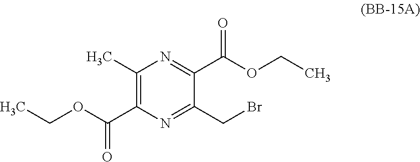 [SMILES: CCOC(=O)c1nc(CBr)c(C(=O)OCC)nc1C]

To a stirred solution of 2,5-diethyl 3,6-dimethylpyrazine-2,5-dicarboxylate (500 mg, 1.90 mmol) in acetonitrile (6.0 mL) were added 1-bromopyrrolidine-2,5-dione (373 mg, 2.09 mmol) and AIBN (31.9 mg, 0.190 mmol). The reaction mixture was irradiated with a white lamp (6200 K) for 48 h. The mixture was concentrated under reduced pressure. The crude was purified by flash chromatography on silica gel using a gradient of EtOAc in cyclohexane from 0% to 20% to afford diethyl 3-(bromomethyl)-6-methyl-pyrazine-2,5-dicarboxylate as a white solid (402 mg, 69% p., 44% y.). m/z [M+H]⁺=331.1, ¹H NMR (400 MHz, DMSO-d₆): δ (ppm) 4.94 (s, 2H), 4.42 (m, 4H), 1.67 (s, 3H), 1.36 (tq, J=4.6, 7.1 Hz, 6H).

### Intermediate BB-15B: ethyl 6-[(1S)-4-tert-butoxy-1-carbamoyl-4-oxo-butyl]-2-methyl-7-oxo-5H-pyrrolo[3,4-b]pyrazine-3-carboxylate

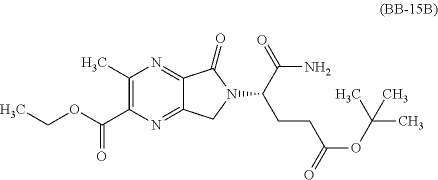 [SMILES: CCOC(=O)c1nc2c(nc1C)C(=O)N([C@@H](CCC(=O)OC(C)(C)C)C(N)=O)C2]

To a stirred solution of diethyl 3-(bromomethyl)-6-methyl-pyrazine-2,5-dicarboxylate (402 mg, 0.838 mmol) in acetonitrile (3.0 mL) at 25° C. were added tert-butyl(4S)-4-amino-4-carbamoylbutanoate hydrochloride (224 mg, 0.921 mmol) and DIPEA (439 μL, 2.51 mmol). The reaction mixture was stirred at 80° C. for 16 h. The reaction was quenched with the addition of a saturated aqueous solution of NaHCO₃. EtOAc was added. The aqueous layer was extracted with EtOAc. The combined organic layers were washed with brine, dried over Na₂SO₄, filtered, and concentrated under reduced pressure. The crude was purified by flash chromatography using a gradient of EtOAc/EtOH (3/1) in cyclohexane from 5% to 50% to afford ethyl 6-[(1S)-4-tert-butoxy-1-carbamoyl-4-oxo-butyl]-2-methyl-7-oxo-5H-pyrrolo[3,4-b]pyrazine-3-carboxylate as a yellow solid (204 mg, 92% p., 55% y.). m/z [M+Na]⁺=429.3, ¹H NMR (400 MHz, DMSO-d₆): δ (ppm) 7.58 (s, 1H), 7.27 (s, 1H), 4.82 (dd, J=3.7, 10.6 Hz, 1H), 4.59 (s, 2H), 4.43 (q, J=7.1 Hz, 2H), 2.78 (s, 3H), 2.24 (m, 3H), 2.01 (m, 1H), 1.38 (m, 3H), 1.34 (s, 9H).

### Intermediate BB-15

To a stirred solution of ethyl 6-[(1S)-4-tert-butoxy-1-carbamoyl-4-oxo-butyl]-2-methyl-7-oxo-5H-pyrrolo[3,4-b]pyrazine-3-carboxylate (204 mg, 0.462 mmol) in THF (2.5 mL) at 0° C. was added a 0.5 M aqueous solution of LiOH (970 μL, 0.485 mmol). The reaction mixture was stirred at 0° C. for 20 min. Water and EtOAc were added. The aqueous layer was washed with EtOAc then acidified with a 12 M aqueous solution of HCl until pH 1. The aqueous layer was extracted with EtOAc. The combined organic layers were washed with brine, dried over Na₂SO₄, filtered, and concentrated under reduced pressure to afford 6-[(1S)-4-tert-butoxy-1-carbamoyl-4-oxo-butyl]-2-methyl-7-oxo-5H-pyrrolo[3,4-b]pyrazine-3-carboxylic acid as a white solid (64 mg, 80% p. 29% y.). m/z [M+Na]⁺=401.0.

### INTERMEDIATE BB-16

### 6-[(1S)-4-tert-butoxy-1-carbamoyl-4-oxo-butyl]-2-methyl-7-oxo-5H-pyrrolo[3,4-b]pyridine-3-carboxylic acid

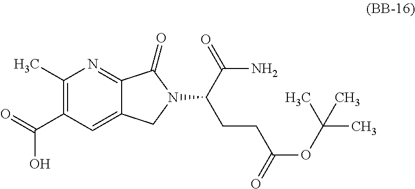 [SMILES: Cc1nc2c(cc1C(=O)O)CN([C@@H](CCC(=O)OC(C)(C)C)C(N)=O)C2=O]

### Intermediate BB-16A: dimethyl 3-methyl-1-oxido-pyridin-1-ium-2,5-dicarboxylate

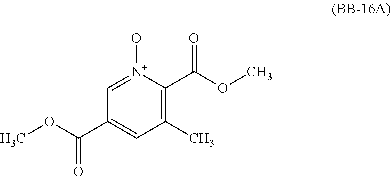

To a stirred solution of dimethyl 3-methylpyridine-2,5-dicarboxylate (3.00 g, 14.3 mmol) in DCM (57 mL) at 0° C. was added 3-chlorobenzenecarboperoxoic acid (8.59 g, 35.9 mmol) portion wise. The reaction mixture was stirred at ambient temperature overnight. Additional 3-chlorobenzenecarboperoxoic acid (3.54 g, 14.3 mmol) was added and the reaction mixture was stirred for 8 h. The reaction mixture was concentrated under reduced pressure. The crude was purified by flash chromatography on silica gel using a gradient EtOAc/EtOH (3/1) in cyclohexane from 10% to 100% to afford dimethyl 3-methyl-1-oxido-pyridin-1-ium-2,5-dicarboxylate as brown oil (2.26 g, 99% p., 69% y.). m/z0. [M+H]⁺=226.2, ¹H NMR (400 MHz, DMSO-d₆): δ (ppm) 8.49 (dd, J=1.4, 0.7 Hz, 1H), 7.83 (dd, J=1.4, 0.8 Hz, 1H), 3.93 (s, 3H), 3.89 (s, 3H), 2.28 (s, 3H).

### Intermediate BB-16B: dimethyl 6-chloro-3-methyl-pyridine-2,5-dicarboxylate

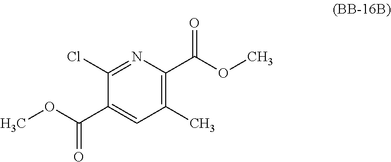

To a stirred solution of dimethyl 3-methyl-1-oxido-pyridin-1-ium-2,5-dicarboxylate (2.26 g, 10.0 mmol) in toluene (56 mL) was added phosphoryl trichloride (1.0 mL, 11.0 mmol). The reaction mixture was stirred at 100° C. overnight. The reaction mixture was cooled to ambient temperature and the mixture was diluted with EtOAc. The reaction was quenched with the addition of a saturated aqueous solution of NaHCO₃. The aqueous layer was extracted with EtOAc (2×). The combined organic layers were washed with water, brine, dried over Na₂SO₄, filtered, and concentrated under reduced pressure to afford dimethyl 6-chloro-3-methyl-pyridine-2,5-dicarboxylate as an orange solid (2.02 g, 87% p., 72% y.). m/z [M+H]⁺=244.2, ¹H NMR (400 MHz, DMSO-d₆): δ (ppm) 8.29 (d, J=0.7 Hz, 1H), 3.90 (d, J=3.2 Hz, 6H), 2.49 (d, J=0.7 Hz, 3H).

### Intermediate BB-16C: dimethyl 3,6-dimethylpyridine-2,5-dicarboxylate

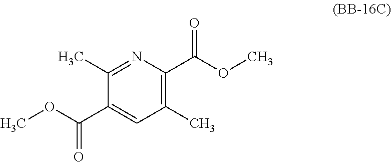

To a stirred solution of dimethyl 6-chloro-3-methyl-pyridine-2,5-dicarboxylate (2.02 g, 8.29 mmol) in 1,4-dioxane (21 mL) under nitrogen were added successively K₂CO₃ (4.58 g, 33.2 mmol), a solution of 2,4,6-trimethyl-1,3,5,2,4,6-trioxatriborinane in THF (50% p., 2.6 mL, 9.12 mmol) and then Pd(PPh₃)₄ (958 mg, 0.829 mmol). The reaction mixture was stirred at 100° C. overnight. The reaction mixture was filtered 10 through a glass fiber filter and washed with EtOAc. The organic layer was washed with a saturated aqueous solution of NaHCO₃. The aqueous layer was extracted with EtOAc. The combined organic layers were washed with brine, dried over Na₂SO₄, filtered, and concentrated under reduced pressure. The crude was purified by flash chromatography on silica gel using a gradient of EtOAc in cyclohexane from 2% to 50% to afford 3,6-dimethylpyridine-2,5-dicarboxylate as a yellow solid (730 mg, 96% p., 38% y.). m/z= [M+H]⁺=224.2, ¹H NMR (400 MHz, DMSO-d₆): δ (ppm) 8.15 (s, 1H), 3.88 (d, J=1.8 Hz, 6H), 2.67 (s, 3H), 2.42 (t, J=0.7 Hz, 3H).

### Intermediate BB-16D: dimethyl 3-(bromomethyl)-6-methyl-pyridine-2,5-dicarboxylate

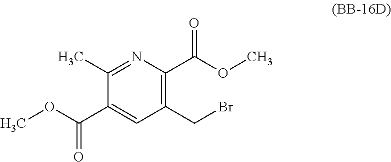

To a stirred solution of dimethyl 3,6-dimethylpyridine-2,5-dicarboxylate (730 mg, 3.27 mmol) in acetonitrile (16 mL) were added 1-bromopyrrolidine-2,5-dione (640 mg, 3.60 mmol) and AIBN (53.7 mg, 0.327 mmol). The reaction mixture was irradiated with a white lamp (6200 K) overnight. The mixture was concentrated under reduced pressure. The crude was purified by flash chromatography on silica gel using a gradient of EtOAc in cyclohexane from 2% to 50% to afford dimethyl 6-(bromomethyl)-3-methyl-pyridine-2,5-dicarboxylate as a pale yellow oil (843 mg, 49% p., 42% y.). m/z [M+H]⁺=no ionization.

### Intermediate BB-16E: methyl 6-[(1S)-4-tert-butoxy-1-carbamoyl-4-oxo-butyl]-2-methyl-7-oxo-5H-pyrrolo[3,4-b]pyridine-3-carboxylate

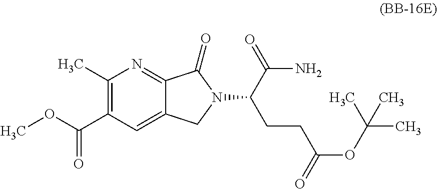 [SMILES: COC(=O)c1cc2c(nc1C)C(=O)N([C@@H](CCC(=O)OC(C)(C)C)C(N)=O)C2]

To a stirred suspension of tert-butyl(4S)-4-amino-4-carbamoylbutanoate hydrochloride (706 mg, 2.96 mmol) in acetonitrile (4.6 mL) at 0° C. was added DIPEA (975 μL, 5.58 mmol) dropwise. The reaction mixture was stirred for 30 min at 0° C. A solution of dimethyl 3-(bromomethyl)-6-methyl-pyridine-2,5-dicarboxylate (843 mg, 2.79 mmol) in acetonitrile (8.0 mL) was added dropwise over 30 min at 0° C. The reaction mixture was stirred to ambient temperature for 1 h then at 70° C. overnight. The reaction mixture was cooled to ambient temperature. The reaction was quenched with the addition of a saturated aqueous solution of NH₄Cl. The aqueous layer was extracted with EtOAc (3×). The combined organic layers were washed with brine, dried over Na₂SO₄, filtered, and concentrated under reduced pressure. The crude was purified by flash chromatography on silica gel using a gradient of EtOAc/EtOH (3/1) in cyclohexane from 2% to 100% to afford methyl 6-[(1S)-4-tert-butoxy-1-carbamoyl-4-oxo-butyl]-2-methyl-7-oxo-5H-pyrrolo[3,4-b]pyridine-3-carboxylate as a yellow solid (690 mg, 70% p., 44% y.). m/z [M+H]⁺=392.3.

### Intermediate BB-16

To a stirred solution of methyl 6-[(1S)-4-tert-butoxy-1-carbamoyl-4-oxo-butyl]-2-methyl-7-oxo-5H-pyrrolo[3,4-b]pyridine-3-carboxylate (690 mg, 1.76 mmol) in THF (5.9 mL) was added a 2 M aqueous solution of LiOH (925 μL, 1.85 mmol). The reaction mixture was stirred at ambient temperature for 2 h. The crude was purified by reverse-phase preparative chromatography using a gradient of acetonitrile in water from 0% to 100% (0.1% AcOH) to afford [(1S)-4-tert-butoxy-1-carbamoyl-4-oxo-butyl]-2-methyl-7-oxo-5H-pyrrolo[3,4-b]pyridine-3-carboxylic acid as a white solid (52.9 mg, 65% p., 5% y.). m/z [M+H]⁺=378.3.

### INTERMEDIATE BB-17

### 2-[(1S)-4-tert-butoxy-1-carbamoyl-4-oxo-butyl]-6-[(dimethylamino)methyl]-1-oxo-isoindoline-5-carboxylic acid acetic acid

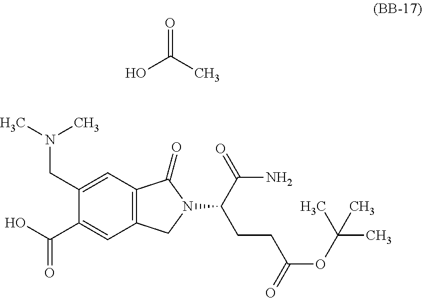

### Intermediate BB-17A: methyl 2-[(1S)-4-tert-butoxy-1-carbamoyl-4-oxo-butyl]-6-[(dimethylamino)methyl]-1-oxo-isoindoline-5-carboxylate

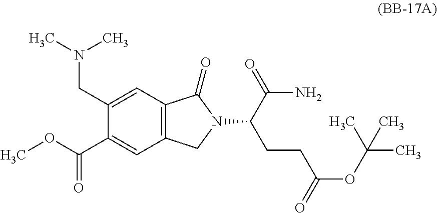 [SMILES: COC(=O)c1cc2c(cc1CN(C)C)C(=O)N([C@@H](CCC(=O)OC(C)(C)C)C(N)=O)C2]

To a stirred suspension of dimethyl 2,5-bis(bromomethyl)benzene-1,4-dicarboxylate (565 mg, 1.49 mmol) in acetonitrile (15 mL) at 0° C. was added DIPEA (1.3 mL, 7.43 mmol) and then a 2 M solution of N-methylmethanamine in THF (743 μL, 1.49 mmol) dropwise. The reaction mixture was stirred for 1.5 h at 0° C. and for 1 h at ambient temperature. The reaction mixture was cooled to 0° C. and tert-butyl(4S)-4-amino-4-carbamoylbutanoate hydrochloride (373 mg, 1.56 mmol) was added portion wise. The reaction mixture was stirred for 1 h at 0° C. and for 1 h at ambient temperature then at 70° C. overnight. The reaction mixture was cooled to ambient temperature. The reaction was quenched with the addition of a saturated aqueous solution of NH₄Cl. The aqueous layer was extracted with EtOAc (3×). The combined organic layers were washed with brine, dried over sodium sulfate, filtered, and concentrated under reduced pressure. The crude was purified by reverse-phase preparative chromatography using a gradient of acetonitrile in water from 0% to 100% (0.1% AcOH). Select fractions were poured in a saturated aqueous solution of NaHCO₃. The aqueous layer was extracted with EtOAc (3×). The combined organic layers were washed with brine, dried over sodium sulfate, filtered, and concentrated under reduced pressure to afford methyl 2-[(1S)-4-tert-butoxy-1-carbamoyl-4-oxo-butyl]-6-[(dimethylamino)methyl]-1-oxo-isoindoline-5-carboxylate as a white solid (93.8 mg, 92% p., 13% y.). m/z [M+H]⁺=434.3, ¹H NMR (400 MHz, DMSO-d₆): δ (ppm) 7.82 (s, 1H), 7.72 (s, 1H), 7.58 (s, 1H), 7.20 (s, 1H), 4.82-4.70 (m, 1H), 4.61 (d, J=17.8 Hz, 1H), 4.49 (d, J=17.9 Hz, 1H), 3.84 (s, 3H), 3.74-3.52 (m, 2H), 2.24-2.05 (m, 9H), 2.04-1.89 (m, 1H), 1.32 (s, 9H).

### Intermediate BB-17

To a stirred solution of methyl 2-[(1S)-4-tert-butoxy-1-carbamoyl-4-oxo-butyl]-6-[(dimethylamino)methyl]-1-oxo-isoindoline-5-carboxylate (93.0 mg, 0.215 mmol) in THF (1.4 mL) and water (340 μL) at 0° C. was added a 2 M aqueous solution of LiOH (113 μL, 0.225 mmol). The reaction mixture was stirred at 0° C. for 4 h. Additional 2 M aqueous solution of LiOH (113 μL, 0.225 mmol) was added. The reaction mixture was stirred at 0° C. for 4 h. The reaction was quenched with the addition of AcOH and purified by reverse-phase preparative chromatography using a gradient of acetonitrile in water from 0% to 100% (0.1% AcOH in water) to afford acetic acid 2-[(1S)-4-tert-butoxy-1-carbamoyl-4-oxo-butyl]-6-[(dimethylamino)methyl]-1-oxo-isoindoline-5-carboxylic acid as a colorless gum (23.6 mg, 70% p., 16% y.). m/z [M+H]⁺=420.3.

### INTERMEDIATE BB-18

### benzyl 4-[(3-bromo-2-chloro-phenyl)-phenyl-methyl]piperidine-1-carboxylate

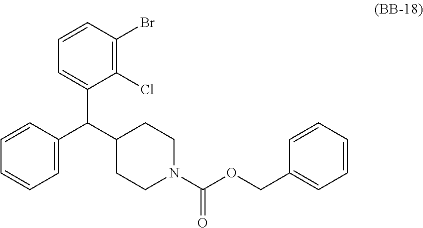 [SMILES: O=C(OCc1ccccc1)N1CCC(C(c2ccccc2)c2cccc(Br)c2Cl)CC1]

### Intermediate BB-18A: benzyl 4-[(3-bromo-2-chloro-phenyl)-hydroxy-phenyl-methyl]piperidine-1-carboxylate

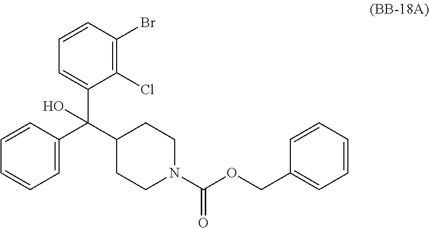 [SMILES: O=C(OCc1ccccc1)N1CCC(C(O)(c2ccccc2)c2cccc(Br)c2Cl)CC1]

To a stirred solution of 1,3-dibromo-2-chlorobenzene (10.2 g, 37.2 mmol) and benzyl 4-benzoylpiperidine-1-carboxylate (10.0 g, 30.9 mmol) in dry THF (150 mL) at −78° C. under nitrogen was added a 1.6 M solution of n-butyllithium in hexanes (29 mL, 46.4 mmol). The reaction mixture was stirred at −70° C. for 10 min and at ambient temperature for 12 h. The reaction was quenched with the addition of a saturated aqueous solution of NH₄Cl, water. EtOAc were added. The phases were separated, and the aqueous phase extracted with EtOAc. The combined organic layers were washed with brine, dried using a phase separator and concentrated under reduced pressure. The crude was solubilized in a minimum of DCM and added to a stirring solution of cyclohexane. The resulting precipitate was filtered, washed with cyclohexane, and dried under reduced pressure to afford benzyl 4-[(3-bromo-2-chloro-phenyl)-hydroxy-phenyl-methyl]piperidine-1-carboxylate as a white solid (7.26 g, 89% p., 41% y.). m/z [M+H]⁺=514.0, ¹H NMR (400 MHz, DMSO-d₆): δ (ppm) 8.05 (dd, J=8.0, 1.6 Hz, 1H), 7.66 (dd, J=8.0, 1.5 Hz, 1H), 7.43-7.20 (m, 10H), 7.20-7.13 (m, 1H), 5.66 (s, 1H), 5.05 (s, 2H), 4.03 (d, J=12.9 Hz, 2H), 3.15 (s, 1H), 2.88 (s, 3H), 1.55 (s, 1H), 1.40-1.07 (m, 2H).

### Intermediate BB-18

To a stirred solution of triethylsilane (6.1 mL, 37.7 mmol) in DCM (40 mL) at −15° C. under nitrogen was added TFA (29 mL, 0.379 mol) dropwise. A solution of benzyl 4-[(3-bromo-2-chloro-phenyl)-hydroxy-phenyl-methyl]piperidine-1-carboxylate (7.26 g, 12.6 mmol) in DCM (75 mL) was added dropwise (keeping the temperature below −10° C.). The reaction mixture was allowed to warm up to ambient temperature for 3 h. The reaction mixture was basified with a 2 M aqueous solution of NaOH (189 mL, 0.379 mol) at 0° C. The aqueous phase was extracted with EtOAc (2×). The combined organic layers were washed with brine, dried using hydrophobic paper and concentrated under reduced pressure. The crude material was purified by flash chromatography on silica gel using a gradient of EtOAc in n-heptane from 0% to 15%. Select fractions were concentrated under reduced pressure to afford benzyl 4-[(3-bromo-2-chloro-phenyl)-phenyl-methyl]piperidine-1-carboxylate as colorless gum (4.84 g, 93% p., 72% y). m/z [M+H]⁺=498.1, ¹H NMR (400 MHz, DMSO-d₆): δ (ppm) 7.76 (dd, J=7.9, 1.5 Hz, 1H), 7.61 (dd, J=7.9, 1.4 Hz, 1H), 7.39-7.24 (m, 10H), 7.23-7.14 (m, 1H), 5.05 (s, 2H), 4.19 (d, J=11.2 Hz, 1H), 3.97 (d, J=13.4 Hz, 2H), 2.78 (s, 2H), 2.46 (d, J=11.1 Hz, 1H), 1.47 (d, J=13.1 Hz, 1H), 1.35 (d, J=13.1 Hz, 1H), 1.12-0.93 (m, 2H).

### INTERMEDIATE BB-19

### benzyl 4-[(R)-(3-bromo-2-chloro-phenyl)-phenyl-methyl]piperidine-1-carboxylate

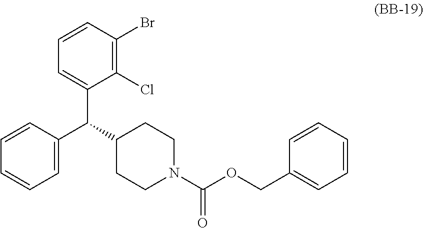 [SMILES: O=C(OCc1ccccc1)N1CCC([C@H](c2ccccc2)c2cccc(Br)c2Cl)CC1]

To a stirred solution of triethylsilane (4.0 mL, 24.8 mmol) and TFA (19 mL, 0.248 mol) in DCM (90 mL) at −15° C. was added a solution of benzyl 4-[(3-bromo-2-chloro-phenyl)-hydroxy-phenyl-methyl]piperidine-1-carboxylate (4.26 g, 8.27 mmol) in DCM (23 mL) dropwise. The mixture was stirred at −15° C. for 2 h and then warmed up to ambient temperature overnight. The reaction mixture was slowly poured into a cooled 1 M aqueous solution of NaOH (248 mL, 0.248 mol) at 0° C. The layers were separated, and the aqueous layer extracted with DCM (2×). The combined organic layers were dried over Na₂SO₄, filtered, and concentrated under reduced pressure. The crude was purified by flash chromatography on silica gel using a gradient of EtOAc in cyclohexane from 1% to 60% to afford benzyl 4-[(3-bromo-2-chloro-phenyl)-phenyl-methyl]piperidine-1-carboxylate as an off-white foam (1.89 g, 97% p., 44% y.) and then, by chiral reverse-phase (Novasep, ChiralCel OD (20 μm, 300× 76.6 mm), MeOH as mobile phase, flow rate 275 mL/min) to afford benzyl 4-[(R)-(3-bromo-2-chloro-phenyl)-phenyl-methyl]piperidine-1-carboxylate (second isomer eluted) as a yellow gum (849 mg, 93% p., 19% y.). m/z [M+H]⁺=498.1, ¹H NMR (400 MHz, DMSO-d₆): δ (ppm) 7.75 (dd, J=7.9, 1.5 Hz, 1H), 7.60 (dd, J=7.9, 1.4 Hz, 1H), 7.39-7.22 (m, 9H), 7.21-7.15 (m, 1H), 5.05 (s, 2H), 4.18 (d, J=11.1 Hz, 1H), 3.97 (d, J=13.3 Hz, 2H), 2.77 (brs, 2H), 2.54-2.44 (m, 2H), 1.46 (d, J=13.1 Hz, 1H), 1.34 (d, 13.1 Hz, 1H), 1.13-0.90 (m, 2H), [α]_{D}=+57° mL·dm⁻¹ g⁻¹[20° C., Na lamp, 589 nm, C=3.9 mg/mL, EtOH].

### INTERMEDIATE BB-20

### Benzyl 4-[(S)-(3-bromo-2-chloro-phenyl)-phenyl-methyl]piperidine-1-carboxylate

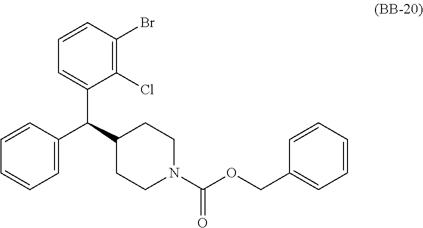 [SMILES: O=C(OCc1ccccc1)N1CCC([C@@H](c2ccccc2)c2cccc(Br)c2Cl)CC1]

To a stirred solution of triethylsilane (4.0 mL, 24.8 mmol) and TFA (19 mL, 0.248 mol) in DCM (90 mL) at −15° C. was added a solution of benzyl 4-[(3-bromo-2-chloro-phenyl)-hydroxy-phenyl-methyl]piperidine-1-carboxylate (4.26 g, 8.27 mmol) in DCM (23 mL) dropwise. The mixture was stirred at −15° C. for 2 h and then warmed up to ambient temperature overnight. The reaction mixture was slowly poured into a cooled 1 M aqueous solution of NaOH (248 mL, 0.248 mol) at 0° C. The layers were separated, and the aqueous layer extracted with DCM (2×). The combined organic layers were dried over Na₂SO₄, filtered, and concentrated under reduced pressure. The crude was purified by flash chromatography on silica gel using a gradient of EtOAc in cyclohexane from 1% to 60% to afford benzyl 4-[(3-bromo-2-chloro-phenyl)-phenyl-methyl]piperidine-1-carboxylate as an off-white foam (1.89 g, 97% p., 44% y.)) then, by chiral reverse-phase (Novasep, ChiralCel OD (20 μm, 300×76.6 mm), MeOH as mobile phase, flow rate 275 mL/min) to afford benzyl 4-[(S)-(3-bromo-2-chloro-phenyl)-phenyl-methyl]piperidine-1-carboxylate (first isomer eluted) as an off-white foam (873 mg, 95% p., 20% y.). m/z [M+H]⁺=498.1, ¹H NMR (400 MHz, DMSO-d₆): δ (ppm) 7.76 (dd, J=8.0, 1.5 Hz, 1H), 7.61 (dd, J=8.0, 1.4 Hz, 1H), 7.41-7.24 (m, 9H), 7.24-7.11 (m, 1H), 5.05 (s, 2H), 4.19 (d, J=11.2 Hz, 1H), 3.97 (d, J=13.1 Hz, 2H), 2.77 (brs, 2H), 2.51-2.47 (m, 2H), 1.47 (d, J=13.1 Hz, 1H), 1.35 (d, J=13.1 Hz, 1H), 1.03 (dqd, J=24.4, 12.4, 4.2 Hz, 2H), [α]_{D}=−50° mL·dm⁻¹.g⁻¹[20° C., Na lamp, 589 nm, C=7.0 mg/mL, EtOH].

### INTERMEDIATE BB-21

### benzyl 4-[bromo (phenyl)methylene]piperidine-1-carboxylate

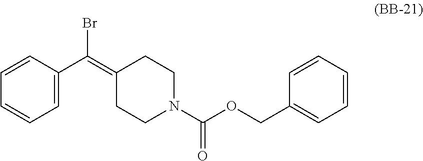 [SMILES: O=C(OCc1ccccc1)N1CCC(=C(Br)c2ccccc2)CC1]

### Intermediate BB-21A: benzyl 4-benzylidenepiperidine-1-carboxylate

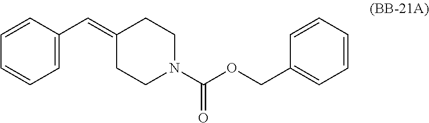 [SMILES: O=C(OCc1ccccc1)N1CCC(=Cc2ccccc2)CC1]

To a stirred solution of benzyltriphenylphosphonium chloride (15.0 g, 38.5 mmol) in dry THF (125 mL) at −78° C. was added a 1.6 M solution of n-butyllithium in hexanes (27 mL, 42.2 mmol) dropwise. The reaction mixture was allowed to warm up to ambient temperature and stirred until the solution became red/orange. The reaction mixture was cooled to −78° C. and a solution of benzyl 4-oxopiperidine-1-carboxylate (16.3 g, 69.7 mmol) in dry THF (63 mL) was added dropwise. The reaction mixture was allowed to warm up to ambient temperature and stirred overnight. The reaction was quenched with the addition of water and EtOAc was added. The reaction mixture was washed with a saturated aqueous solution of NH₄Cl then brine, dried over Na₂SO₄, filtered, and the solvent was concentrated under reduced pressure. The crude was purified by flash chromatography on silica gel using a gradient of EtOAc in cyclohexane from 0% to 20% to afford benzyl 4-benzylidenepiperidine-1-carboxylate as a colorless oil (7.86 g, 100% p., 66% y.). m/z [M+H]⁺=308.3, ¹H NMR (400 MHz, DMSO-d₆): δ (ppm) 7.44-7.28 (m, 7H), 7.26-7.17 (m, 3H), 6.39 (s, 1H), 5.10 (s, 2H), 3.51 (s, 2H), 3.43 (s, 2H), 2.46-2.39 (m, 2H), 2.32 (td, J=5.6, 1.3 Hz, 2H).

### Intermediate BB-21

To a stirred solution of benzyl 4-benzylidenepiperidine-1-carboxylate (7.86 g, 25.6 mmol) in chloroform (74 mL) at 0° C. was added K₂CO₃ (5.30 g, 38.4 mmol) then a solution of molecular bromine (1.6 mL, 30.7 mmol) in CHCl₃ (12 mL) was added dropwise. The reaction mixture was stirred at ambient temperature overnight. DCM was added and the reaction mixture was washed with a saturated aqueous solution of NH₄Cl then brine, dried over Na₂SO₄, filtered, and the solvent was removed under reduced pressure. The product was solubilized in MeOH (118 mL) and a solution of NaOH (10.2 g, 256 mmol) in water (44 mL) was added. The reaction mixture was stirred at 40° C. for 2 h. DCM was added and the reaction mixture was washed with a 12 M aqueous solution of HCl, brine, dried over Na₂SO₄, filtered, and the solvent was removed under reduced pressure. The crude was purified by flash chromatography on silica gel using a gradient of EtOAc in cyclohexane from 2% to 40% to afford benzyl 4-[bromo (phenyl)methylene]piperidine-1-carboxylate as a pink oil (8.16 g, 97% p., 80% y.). m/z [M+H]⁺=388.2, ¹H NMR (400 MHz, DMSO-d₆): δ (ppm) 7.45-7.27 (m, 10H), 5.09 (s, 2H), 3.55 (s, 2H), 3.38 (s, 2H), 2.62-2.55 (m, 2H), 2.26-2.14 (m, 2H).

### INTERMEDIATE BB-22

### 6-[(1S)-4-tert-butoxy-1-carbamoyl-4-oxo-butyl]-7-oxo-5H-pyrrolo[3,4-b]pyridine-3-carboxylic acid

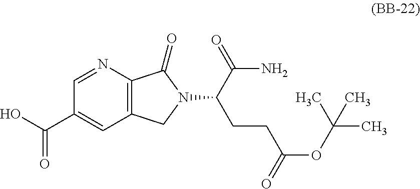 [SMILES: CC(C)(C)OC(=O)CC[C@@H](C(N)=O)N1Cc2cc(C(=O)O)cnc2C1=O]

### Intermediate 22A: methyl 5-bromo-3-(bromomethyl)pyridine-2-carboxylate

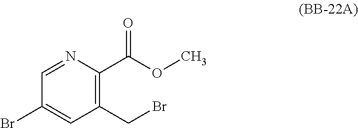

To a stirred solution of methyl 5-bromo-3-methylpyridine-2-carboxylate (3.00 g, 13.0 mmol) in acetonitrile (65 mL) were added 1-bromopyrrolidine-2,5-dione (2.55 g, 14.3 mmol) and AIBN (214 mg, 1.30 mmol). The reaction mixture was irradiated with a white lamp (6200 K) overnight. The mixture was concentrated under reduced pressure. The crude was purified by flash chromatography on silica gel using a gradient of EtOAc in cyclohexane from 2% to 100% to afford methyl 5-bromo-3-(bromomethyl)pyridine-2-carboxylate as a white solid (3.36 g, 88% p., 73% y.). m/z [M+H]⁺=308.0, ¹H NMR (400 MHz, DMSO-d₆): δ (ppm) 8.77 (d, J=2.2 Hz, 1H), 8.41 (d, J=2.2 Hz, 1H), 4.91 (s, 2H), 3.91 (s, 3H).

### Intermediate 22B: tert-butyl(4S)-5-amino-4-(3-bromo-7-oxo-5H-pyrrolo[3,4-b]pyridin-6-yl)-5-oxo-pentanoate

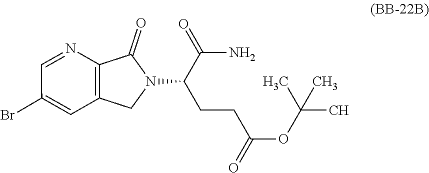 [SMILES: CC(C)(C)OC(=O)CC[C@@H](C(N)=O)N1Cc2cc(Br)cnc2C1=O]

To a stirred suspension of tert-butyl(4S)-4-amino-4-carbamoylbutanoate hydrochloride (2.75 g, 11.5 mmol) in acetonitrile (18 mL) at 0° C. was added DIPEA (3.8 mL, 21.8 mmol) dropwise. The reaction mixture was stirred for 30 min at 0° C. A solution of methyl 5-bromo-3-(bromomethyl)pyridine-2-carboxylate (3.36 g, 10.9 mmol) in acetonitrile (31 mL) was added dropwise over 30 min at 0° C. The reaction mixture was then stirred at 70° C. overnight. The reaction mixture was cooled to ambient temperature. The reaction was quenched with the addition of a saturated aqueous solution of NH₄Cl. The aqueous layer was extracted with EtOAc (3×). The combined organic layers were washed with brine, dried over Na₂SO₄, filtered, and concentrated under reduced pressure. The crude was purified by flash chromatography on silica gel using a gradient of EtOAc/EtOH (3/1) in cyclohexane from 2% to 100% to afford tert-butyl(4S)-5-amino-4-(3-bromo-7-oxo-5H-pyrrolo[3,4-b]pyridin-6-yl)-5-oxo-pentanoate as a pale yellow foam (2.62 g, 97% p., 59% y.). m/z [M+H]⁺=398.2, ¹H NMR (400 MHz, DMSO-d₆): δ (ppm) 8.84 (d, 1H), 8.39 (d, 1H), 7.69-7.54 (m, 1H), 7.23 (s, 1H), 4.82-4.71 (m, 1H), 4.60 (d, 1H), 4.49 (d, 1H), 2.24-2.11 (m, 3H), 2.04-1.94 (m, 1H), 1.33 (s, 9H).

### Intermediate BB-22

In a sealed pressure vessel, to a stirred solution of tert-butyl(4S)-5-amino-4-(3-bromo-7-oxo-5H-pyrrolo[3,4-b]pyridin-6-yl)-5-oxo-pentanoate (2.61 g, 6.55 mmol) in DMF (13 mL) were added successively ethanedioic acid dihydrate (1.24 g, 9.83 mmol), XantPhos (190 mg, 0.328 mmol), and Pd(OAc)₂(74 mg, 0.328 mmol). The reaction mixture was degassed with argon for 5 min, and then acetic anhydride (909 μL, 9.83 mmol) and DIPEA (1.7 mL, 9.83 mmol) were added. The reaction mixture was stirred at 100° C. overnight. The reaction mixture was cooled to ambient temperature. The reaction mixture was poured into a 1 M aqueous solution of NaHCO₃. The aqueous layer was washed with EtOAc (3×). The aqueous layer was acidified to pH 3-4 with a 12 M aqueous solution of HCl. The acidic aqueous layer was extracted with EtOAc (3×). The combined organic layers were washed with brine, dried over Na₂SO₄, filtered, and concentrated under reduced pressure to afford 6-[(1S)-4-tert-butoxy-1-carbamoyl-4-oxo-butyl]-7-oxo-5H-pyrrolo[3,4-b]pyridine-3-carboxylic acid as a yellow gum (1.96 g, 98% p., 81% y.). m/z [M+H]⁺=364.3, ¹H NMR (400 MHz, DMSO-d₆): δ (ppm) 13.70 (s, 1H), 9.18 (d, 1H), 8.55 (d, 1H), 7.63 (s, 1H), 7.25 (s, 1H), 4.80 (dd, 1H), 4.66 (d, 1H), 4.55 (d, 1H), 2.28-2.11 (m, 3H), 2.10-1.92 (m, 1H), 1.32 (s, 9H).

### INTERMEDIATE BB-23

### tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate

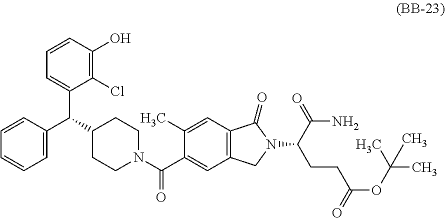 [SMILES: Cc1cc2c(cc1C(=O)N1CCC([C@H](c3ccccc3)c3cccc(O)c3Cl)CC1)CN([C@@H](CCC(=O)OC(C)(C)C)C(N)=O)C2=O]

### Intermediate 23 A: 2-chloro-3-[(R)-phenyl(4-piperidyl)methyl]phenol hydroiodide

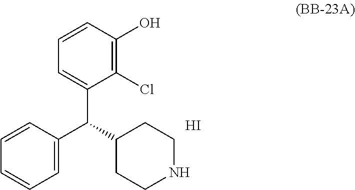

To a stirred solution of benzyl 4-[(R)-(2-chloro-3-hydroxy-phenyl)-phenyl-methyl]piperidine-1-carboxylate (2.18 g, 4.95 mmol) in acetonitrile (24 mL) at ambient temperature was added iodo (trimethyl) silane (2.11 mL, 14.9 mmol) dropwise. The reaction mixture was stirred 10 min at ambient temperature then cooled to 0° C. with an ice bath and MeOH (2.4 mL, 59.4 mmol) was added dropwise. The reaction mixture was stirred at 0° C. for 15 min then concentrated under reduced pressure. Et₂O was added to the crude and the mixture was stirred at ambient temperature for 30 min. The resulting precipitate was filtered, washed with Et₂O, and dried under reduced pressure at 50° C. for 12 h to afford 2-chloro-3-[(R)-phenyl(4-piperidyl)methyl]phenol hydroiodide as a yellow powder (2.08 g, 96% p, 94% y.). m/z [M+H]⁺=302.2, ¹H NMR (400 MHz, DMSO-d₆): δ (ppm) 10.07 (s, 1H), 8.41 (d, J=11.3 Hz, 1H) 8.08 (d, J=11.4 Hz, 1H), 7.37-7.33 (m, 2H), 7.29 (dd, J=8.4, 6.8 Hz, 2H), 7.22-7.16 (m, 1H) 7.14 (t, J=7.8 Hz, 1H), 7.07 (dd, J=7.9, 1.6 Hz, 1H), 6.80 (dd, J=7.9, 1.5 Hz, 1H) 4.12 (d, J=11.2 Hz, 1H), 3.24 (d, J=12.7 Hz, 2H), 2.84 (p, J=11.5 Hz, 2H), 2.61-2.50 (m, 1H), 1.61 (d, J=14.0 Hz, 1H), 1.51 (d, J=14.0 Hz, 1H), 1.33-1.17 (m, 2H), [α]_{D}=+39° mL·dm⁻¹ g⁻¹[20° C., Na lamp, 589 nm, C=3.5 mg/mL, MeOH].

### Intermediate BB-23

To a solution of 2-chloro-3-[(R)-phenyl(4-piperidyl)methyl]phenol hydroiodide (2.17 g, 4.84 mmol) in DMF (24 mL), were added 2-[(1S)-4-tert-butoxy-1-carbamoyl-4-oxo-butyl]-6-methyl-1-oxo-isoindoline-5-carboxylic acid (1.91 g, 5.08 mmol), DIPEA (2.6 mL, 14.5 mmol) and HATU (2.82 g, 7.26 mmol). The mixture was stirred at ambient temperature for 1 h. The reaction was quenched with the addition of a saturated aqueous solution of NaHCO₃. EtOAc was added. The aqueous layer was extracted with EtOAc. The combined organic layers were washed with a saturated aqueous solution of NH₄Cl then brine, filtered over Na₂SO₄, filtered, and concentrated under reduced pressure. The crude was purified by flash chromatography on silica gel using a gradient of EtOAc/EtOH (3/1) (+1% of a 7 N solution of NH₃ in MeOH) in cyclohexane from 20% to 100 to afford tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate as an off-white solid (3.02 g, 100% p., 95% y.). m/z [M+H]⁺=660.3, ¹H NMR (400 MHz, DMSO-d₆): δ (ppm) 9.99 (d, J=22.0 Hz, 1H), 7.95 (s, 1H), 7.55 (d, J=9.7 Hz, 2H), 7.48-7.35 (m, 1H), 7.35-7.05 (m, 6H), 6.77 (dt, J=23.8, 4.7 Hz, 1H), 4.71 (dd, J=10.3, 3.9 Hz, 1H), 4.62-4.35 (m, 3H), 3.27-3.13 (m, 1H), 3.04-2.93 (m, 1H), 2.89 (s, 3H), 2.83-2.73 (m, 3H), 2.33 (d, J=4.5 Hz, 1H), 2.23 (s, 2H), 2.16-2.11 (m, 3H), 2.03-1.91 (m, 1H), 1.51-1.44 (m, 1H), 1.37-1.23 (m, 9H), 1.18-0.89 (m, 1H).

### INTERMEDIATE BB-24

### tert-butyl(4S)-5-amino-4-[5-[4-[(S)-(2-chloro-3-hydroxy-phenyl)-phenyl-methyl]piperidine-1-carbonyl]-6-methyl-1-oxo-isoindolin-2-yl]-5-oxo-pentanoate

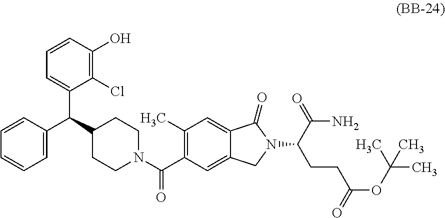 [SMILES: Cc1cc2c(cc1C(=O)N1CCC([C@@H](c3ccccc3)c3cccc(O)c3Cl)CC1)CN([C@@H](CCC(=O)OC(C)(C)C)C(N)=O)C2=O]

### Intermediate 24A: 2-chloro-3-[(S)-phenyl(4-piperidyl)methyl]phenol hydroiodide

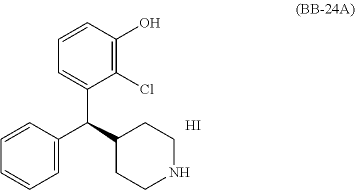

To a stirred solution of benzyl 4-[(S)-(2-chloro-3-hydroxy-phenyl)-phenyl-methyl]piperidine-1-carboxylate (2.34 g, 5.31 mmol) in acetonitrile (25 mL) at ambient temperature under nitrogen was added iodo (trimethyl) silane (2.3 mL, 15.9 mmol) dropwise. The reaction mixture was stirred at ambient temperature for 1 h. MeOH (2.6 mL, 63.8 mmol) was added at 0° C. The mixture was stirred at ambient temperature for 15 min and was concentrated under reduced pressure. Et₂O was added and the resulting mixture was stirred at ambient temperature for 1 h. The solid was filtered, washed with Et₂O, and dried under reduced pressure to afford 2-chloro-3-[(S)-phenyl(4-piperidyl)methyl]phenol hydroiodide as a yellow powder (2.20 g, 98% p., 94% y.). m/z [M+H]⁺=302.2, ¹H NMR (400 MHz, DMSO-d₆): δ (ppm) 10.07 (s, 1H), 8.13 (s, 2H), 7.38-7.25 (m, 4H), 7.23-7.10 (m, 2H), 7.06 (dd, J=7.9, 1.5 Hz, 1H), 6.80 (dd, J=7.9, 1.5 Hz, 1H), 4.12 (d, J=11.2 Hz, 1H), 3.23 (d, J=12.6 Hz, 2H), 2.83 (q, J=10.2 Hz, 2H), 2.55 (s, 1H), 1.61 (d, J=14.3 Hz, 1H), 1.51 (d, J=13.6 Hz, 1H), 1.25 (p, J=12.6 Hz, 2H), [α]_{D}=−41° mL·dm⁻¹.g⁻¹[20° C., Na lamp, 589 nm, C=2.9 mg/mL, MeOH].

### Intermediate BB-24

To a solution of 2-chloro-3-[(S)-phenyl(4-piperidyl)methyl]phenol hydroiodide (2.16 g, 5.02 mmol) in DMF (25 mL), were added 2-[(1S)-4-tert-butoxy-1-carbamoyl-4-oxo-butyl]-6-methyl-1-oxo-isoindoline-5-carboxylic acid (1.98 g, 5.27 mmol), DIPEA (2.6 mL, 15.1 mmol), and HATU (2.92 g, 7.53 mmol). The mixture was stirred at ambient temperature for 1 h. Water, EtOAc, and FastWorX® were added. The organic solvent was evaporated, and the resin was filtered in a solid deposit cartridge. The crude was purified by flash chromatography on silica gel using a gradient of EtOAc/EtOH (3/1) (+1% of a 7 M solution of NH₃ in MeOH) in cyclohexane from 10% to 100%. The desired fractions were combined and concentrated under reduced pressure to afford tert-butyl(4S)-5-amino-4-[5-[4-[(S)-(2-chloro-3-hydroxy-phenyl)-phenyl-methyl]piperidine-1-carbonyl]-6-methyl-1-oxo-isoindolin-2-yl]-5-oxo-pentanoate as an off-white solid (3.40 g, 84% p., 86% y.). m/z [M+Na]⁺=682.3.

### Example 1

### (3S)-3-[5-(4-benzhydrylpiperidine-1-carbonyl)-1-oxo-isoindolin-2-yl]piperidine-2,6-dione

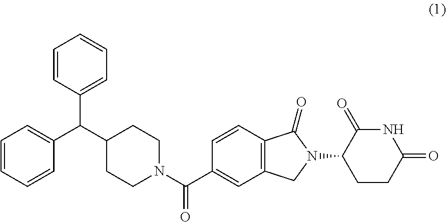 [SMILES: O=C1CC[C@H](N2Cc3cc(C(=O)N4CCC(C(c5ccccc5)c5ccccc5)CC4)ccc3C2=O)C(=O)N1]

### Intermediate 1A: 4-benzhydrylpiperidine

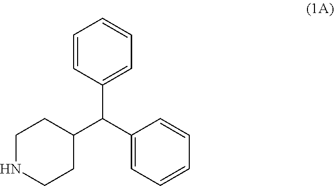 [SMILES: c1ccc(C(c2ccccc2)C2CCNCC2)cc1]

A mixture of sodium borohydride (347 mg, 9.16 mmol) and diphenyl(4-piperidyl) methanol (250 mg, 0.916 mmol) was added portion wise to a stirred solution of TFA (11 mL, 137 mmol) at 0° C. The reaction mixture was stirred at ambient temperature for 45 min. The reaction mixture was concentrated under reduced pressure. The residue was solubilized in DCM. The organic layer was washed with a 1 M aqueous solution of NaOH, brine and then dried over Na₂SO₄, filtered, and concentrated under reduced pressure to afford 4-benzhydrylpiperidine as a white powder (167 mg, 99% p., 72% y.). m/z [M+H]⁺=252.3, ¹H NMR (400 MHz, DMSO-d₆): δ (ppm) 8.55 (brs, 1H), 7.41-7.23 (m, 9H), 7.21-7.11 (m, 2H), 3.60 (d, J=11.2 Hz, 1H), 3.21 (dt, J=13.0, 3.4 Hz, 2H), 2.82 (td, J=12.8, 3.0 Hz, 2H), 1.58-1.49 (m, 2H), 1.32-1.13 (m, 2H).

### Intermediate 1B: tert-butyl(4S)-5-amino-4-[5-(4-benzhydrylpiperidine-1-carbonyl)-1-oxo-isoindolin-2-yl]-5-oxo-pentanoate

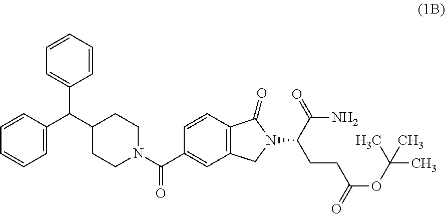 [SMILES: CC(C)(C)OC(=O)CC[C@@H](C(N)=O)N1Cc2cc(C(=O)N3CCC(C(c4ccccc4)c4ccccc4)CC3)ccc2C1=O]

In a sealed vial under nitrogen, to a stirred solution of 4-benzhydrylpiperidine (69.0 mg, 0.276 mmol), 2-[(1S)-4-tert-butoxy-1-carbamoyl-4-oxo-butyl]-1-oxo-isoindoline-5-carboxylic acid (100 mg, 0.276 mmol), and DIPEA (193 μL, 1.10 mmol) in dry EtOAc (2.0 mL) was added a solution of T3P in MeTHF (50% w/w, 263 mg, 0.414 mmol). The mixture was stirred at ambient temperature for 5 h. The reaction was quenched with the addition of a saturated aqueous solution of NaHCO₃. The aqueous layer was extracted with EtOAc (2×). The combined organic layers were washed with brine, dried over Na₂SO₄, filtered, and concentrated under reduced pressure. The crude was purified by flash chromatography on silica gel using a gradient of MeOH in DCM from 0% to 10% to afford tert-butyl(4S)-5-amino-4-[5-(4-benzhydrylpiperidine-1-carbonyl)-1-oxo-isoindolin-2-yl]-5-oxo-pentanoate as a white solid (36.0 mg, 97% p., 21% y.). m/z [M+Na]⁺=618.5, ¹H NMR (400 MHz, DMSO-d₆): δ (ppm) 7.71 (d, J=7.9 Hz, 1H), 7.57 (s, 1H), 7.56 (s, 1H), 7.43 (dd, J=7.8, 1.4 Hz, 1H), 7.42-7.34 (m, 4H), 7.31-7.22 (m, 4H), 7.18 (s, 1H), 7.17-7.08 (m, 2H), 4.77-4.70 (m, 1H), 4.61 (d, J=17.8 Hz, 1H), 4.47 (d, J=18.0 Hz, 2H), 3.63 (d, J=11.2 Hz, 1H), 3.50 (s, 1H), 3.01 (s, 1H), 2.78 (s, 1H), 2.51 (s, 1H), 2.21-2.10 (m, 3H), 2.04-1.91 (m, 1H), 1.54 (s, 1H), 1.31 (s, 10H), 1.12 (t, J=11.8 Hz, 2H).

### Example 1

In a sealed vial, a solution of tert-butyl(4S)-5-amino-4-[5-(4-benzhydrylpiperidine-1-carbonyl)-1-oxo-isoindolin-2-yl]-5-oxo-pentanoate (35.0 mg, 0.0588 mmol) and 4-methylbenzenesulfonic acid hydrate (34.0 mg, 0.177 mmol) in acetonitrile (500 μL) was stirred at 80° C. for 4 h. The reaction was quenched with the addition of a saturated aqueous solution of NaHCO₃. The aqueous layer was extracted with EtOAc (2×). The combined organic layers were washed with brine, dried using hydrophobic paper and concentrated under reduced pressure. The crude was purified by flash chromatography on silica gel using a gradient of EtOAc/EtOH (3/1) in n-heptane from 0% to 100%. Select fractions were concentrated under reduced pressure. The residue was solubilized in a minimum amount of DCM and the solution was added dropwise to stirred solution of n-pentane. The resulting residue was triturated for 3 h, filtered, washed with n-pentane, and dried under reduced pressure at 50° C. for 64 h to afford (3S)-3-[5-(4-benzhydrylpiperidine-1-carbonyl)-1-oxo-isoindolin-2-yl]piperidine-2,6-dione as a white solid (10.0 mg, 96.7% p., 31% y.). m/z [M+H]⁺=522.2, ¹H NMR (500 MHz, DMSO-d₆): δ (ppm) 10.98 (s, 1H), 7.75 (d, J=7.8 Hz, 1H), 7.58 (s, 1H), 7.46 (dd, J=7.7, 1.2 Hz, 1H), 7.43-7.33 (m, 4H), 7.33-7.21 (m, 4H), 7.20-7.07 (m, 2H), 5.12 (dd, J=13.3, 5.2 Hz, 1H), 4.58-4.26 (m, 3H), 3.62 (d, J=11.1 Hz, 1H), 3.57-3.43 (m, 1H), 3.11-2.96 (m, 1H), 2.91 (ddd, J=17.5, 13.6, 5.5 Hz, 1H), 2.84-2.69 (m, 1H), 2.65-2.52 (m, 2H), 2.39 (qd, J=13.2, 4.6 Hz, 1H), 2.08-1.93 (m, 1H) 1.61-1.47 (m, 1H) 1.36 (br s, 1H) 1.11 (br d, J=11.1 Hz, 2H).

### Example 2

### (3S)-3-[5-(4-benzhydrylpiperidine-1-carbonyl)-6-methyl-1-oxo-isoindolin-2-yl]piperidine-2,6-dione

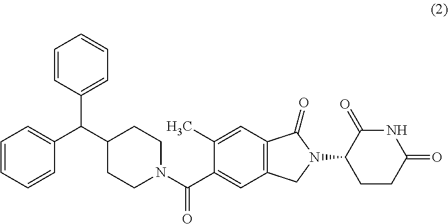 [SMILES: Cc1cc2c(cc1C(=O)N1CCC(C(c3ccccc3)c3ccccc3)CC1)CN([C@H]1CCC(=O)NC1=O)C2=O]

### Intermediate 2A: tert-butyl(4S)-5-amino-4-[5-(4-benzhydrylpiperidine-1-carbonyl)-6-methyl-1-oxo-isoindolin-2-yl]-5-oxo-pentanoate

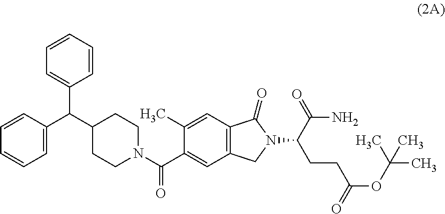 [SMILES: Cc1cc2c(cc1C(=O)N1CCC(C(c3ccccc3)c3ccccc3)CC1)CN([C@@H](CCC(=O)OC(C)(C)C)C(N)=O)C2=O]

In a round-bottom flask, to a stirred suspension of 2-[(1S)-4-tert-butoxy-1-carbamoyl-4-oxo-butyl]-6-methyl-1-oxo-isoindoline-5-carboxylic acid (70 mg, 0.177 mmol) in EtOAc (1.7 mL) were added successively 4-benzhydrylpiperidine (53 mg, 0.212 mmol), DIPEA (93 μL, 0.530 mmol), and a solution of T3P in MeTHF (50% w/w, 169 mg, 0.265 mmol). The reaction mixture was stirred at ambient temperature for 3 h. Additional solution of T3P in MeTHF (50% w/w, 169 mg, 0.265 mmol) was added and the reaction mixture was stirred at ambient temperature overnight. The reaction was quenched with the addition of water. The layers were separated. The organic layer was washed with water (3×), with brine, dried using a phase separator and concentrated under reduced pressure. The crude was purified by flash chromatography on silica gel using a gradient of EtOAc/EtOH (3/1) in cyclohexane from 5% to 80%. The desired fractions were combined and concentrated under reduced pressure. The resulting residue was solubilized in DCM and the organic layer was washed with a saturated aqueous solution of NaHCO₃. The aqueous layer was extracted with DCM (2×). The combined organic layers were washed with brine, dried using a phase separator cartridge and concentrated under reduced pressure to afford tert-butyl(4S)-5-amino-4-[5-(4-benzhydrylpiperidine-1-carbonyl)-6-methyl-1-oxo-isoindolin-2-yl]-5-oxo-pentanoate as a pale yellow foam (36.0 mg, 94% p.,31% y.). m/z [M+H]⁺=610.6, ¹H NMR (400 MHz, DMSO-d₆): δ (ppm) 7.56 (s, 2H), 7.41-7.09 (m, 12H), 4.71 (d, J=10.3 Hz, 1H), 4.50 (dq, J=33.4, 16.8 Hz, 3H), 3.61 (d, J=10.6 Hz, 1H), 3.24 (d, J=13.8 Hz, 1H), 2.97 (t, J=12.9 Hz, 1H), 2.77 (s, 1H), 2.51 (s, 2H), 2.32 (s, 1H), 2.24 (s, 2H), 2.14 (s, 3H), 1.98 (d, J=9.0 Hz, 1H), 1.55 (s, 1H), 1.31 (d, J=18.6 Hz, 9H), 1.06 (d, J=12.6 Hz, 2H).

### Example 2

In a sealed vial, a solution of tert-butyl(4S)-5-amino-4-[5-(4-benzhydrylpiperidine-1-carbonyl)-6-methyl-1-oxo-isoindolin-2-yl]-5-oxo-pentanoate (34.0 mg, 0.0558 mmol) and 4-methylbenzenesulfonic acid hydrate (31.8 mg, 0.167 mmol) in acetonitrile (340 μL) was stirred at 80° C. for 16 h. The mixture was cooled to ambient temperature. The reaction was quenched with the addition of a saturated aqueous solution of NaHCO₃. The aqueous layer was extracted with EtOAc (2×). The combined organic layers were washed with brine. Layers were separated using hydrophobic paper and the filtrate was concentrated under reduced pressure. The crude was purified by flash chromatography on silica gel using a gradient of EtOAc/EtOH (3/1) in n-heptane from 0% to 100%. Fractions were concentrated under reduced pressure. The resulting solid was solubilized in the minimum amount of DCM and the homogenous solution was added dropwise to a stirred solution of n-pentane. The mixture was stirred for 4 h. The resulting precipitate was filtered, washed with n-pentane, and dried under reduced pressure at 50° C. for 16 h to afford (3S)-3-[5-(4-benzhydrylpiperidine-1-carbonyl)-6-methyl-1-oxo-isoindolin-2-yl]piperidine-2,6-dione as a white solid (6.0 mg, 97.4% p., 19% y.). m/z [M+H]⁺=536.4, ¹H NMR (600 MHz, DMSO-d₆): δ (ppm) 10.97 (br s, 1H), 7.60 (d, 1H, J=4.7 Hz), 7.4-7.3 (m, 5H), 7.3-7.2 (m, 4H), 7.2-7.1 (m, 2H), 5.1-5.0 (m, 1H), 4.6-4.5 (m, 1H), 4.5-4.2 (m, 2H), 3.7-3.6 (m, 1H), 3.3-2.7 (m, 4H), 2.6-2.5 (m, 2H), 2.4-2.2 (m, 4H), 2.1-1.9 (m, 1H), 1.6-0.9 (m, 4H).

### Example 3

### (3S)-3-[5-[(1R,5S)-3-benzhydryl-8-azabicyclo[3.2.1]octane-8-carbonyl]-1-oxo-isoindolin-2-yl]piperidine-2,6-dione

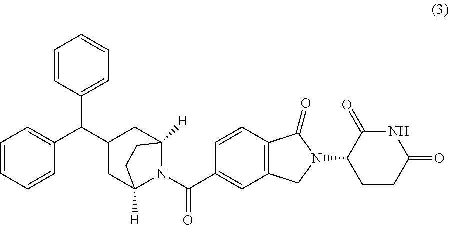 [SMILES: O=C1CC[C@H](N2Cc3cc(C(=O)N4[C@@H]5CC[C@H]4CC(C(c4ccccc4)c4ccccc4)C5)ccc3C2=O)C(=O)N1]

### Intermediate 3A: tert-butyl(1R,5S)-3-benzhydryl-3-hydroxy-8-azabicyclo[3.2.1]octane-8-carboxylate

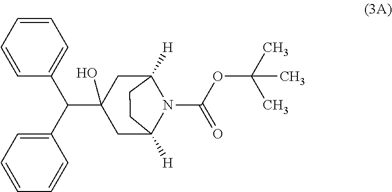 [SMILES: CC(C)(C)OC(=O)N1[C@@H]2CC[C@H]1CC(O)(C(c1ccccc1)c1ccccc1)C2]

A round-bottom flask was charged with benzhydryl bromide (1,30 g, 4.74 mmol) and N-Boc-nortropinone (1,00 g, 4.31 mmol) in dry THF (21 mL) under nitrogen. The mixture was cooled to −78° C. and a 2.5 M solution of n-butyllithium in hexanes (3.8 mL, 9.47 mmol) was added dropwise over 20 min. The resulting mixture was stirred at −78° C. for 1 h. The reaction was quenched with the addition of a saturated aqueous solution of NH₄Cl. The mixture was extracted with EtOAc (3×). The combined organic layers were washed with water, brine, dried over Na₂SO₄, filtered, and concentrated under reduced pressure. The crude was purified on silica gel using a gradient of a mixture of EtOAc/EtOH (3/1) in cyclohexane from 0% to 70% to afford tert-butyl(1R,5S)-3-benzhydryl-3-hydroxy-8-azabicyclo[3.2.1]octane-8-carboxylate as a white powder (470 mg, 93% p., 26% y.). m/z [M−Boc+2H]⁺=338.4, ¹H NMR (400 MHz, DMSO-d₆): δ (ppm) 7.52-7.45 (m, 4H), 7.25 (dd, J=8.3, 6.9 Hz, 4H), 7.18-7.12 (m, 2H), 4.43 (s, 1H), 3.99 (d, J=6.1 Hz, 2H), 3.51 (s, 1H), 2.12 (d, J=6.6 Hz, 2H), 1.70 (d, J=41.3 Hz, 3H), 1.44 (s, 3H), 1.39 (s, 9H).

### Intermediate 3B: (1R,5S)-3-benzhydryl-8-azabicyclo[3.2.1]octan-3-ol

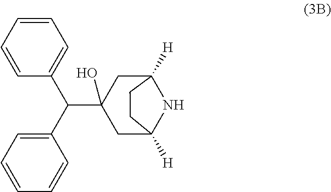 [SMILES: OC1(C(c2ccccc2)c2ccccc2)C[C@H]2CC[C@@H](C1)N2]

To a stirred solution of tert-butyl(1R,5S)-3-benzhydryl-3-hydroxy-8-azabicyclo[3.2.1]octane-8-carboxylate (235 mg, 0.554 mmol) in EtOH (3.0 mL) at ambient temperature were added successively triethylamine (270 μL, 1.67 mmol), palladium on carbon (10% w/w, 59 mg, 0.0555 mmol) and AcOH (32 μL, 0.556 mmol). The reaction mixture was stirred at 80° C. overnight. Additional palladium on carbon (10% w/w, 59 mg, 0.0555 mmol), AcOH (32 μL, 0.556 mmol), and triethylamine (270 μL, 1.67 mmol) were added. The reaction mixture was stirred at 80° C. for 5 h and then at ambient temperature for 72 h. The reaction mixture was filtered through a pad of celite and the filtrate was concentrated under reduced pressure. The crude was diluted in dry DCE (3.0 mL). Triethylsilane (272 μL, 1.67 mmol) and TFA (128 μL, 16.7 mmol) were added. The reaction mixture was stirred at ambient temperature overnight. EtOAc was added and the reaction mixture was washed with a saturated aqueous solution of NaHCO₃then brine, dried over Na₂SO₄, and filtered. The solvent was removed under reduced pressure to afford (1R,5S)-3-benzhydryl-8-azabicyclo[3.2.1]octan-3-ol as a colorless oil (170 mg, 96% p., quant. yield). m/z [M+H]⁺=294.3.

### Intermediate 3C: (1R,5S)-3-benzhydryl-8-azabicyclo[3.2.1]oct-2-ene

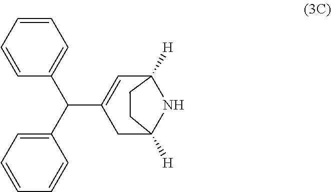 [SMILES: C1=C(C(c2ccccc2)c2ccccc2)C[C@H]2CC[C@@H]1N2]

To a stirred solution of (1R,5S)-3-benzhydryl-8-azabicyclo[3.2.1]octan-3-ol (170 mg, 0.579 mmol) in dry THF (2.9 mL) at ambient temperature was added sulfuric acid (618 μL, 11.6 mmol). The reaction mixture was stirred at 70° C. overnight. EtOAc was added and the reaction mixture was washed with a saturated aqueous solution of NaHCO₃then brine, dried over Na₂SO₄, filtered, and the solvent was removed under reduced pressure. The crude was purified by flash chromatography on silica gel using a gradient of EtOAc in n-heptane from 0% to 100% to afford (1R,5S)-3-benzhydryl-8-azabicyclo[3.2.1]oct-2-ene as a yellow oil (103 mg, 98% p., 63% y.). m/z [M+H]⁺=276.4, ¹H NMR (400 MHz, DMSO-d₆): δ (ppm) 7.35-7.27 (m, 4H), 7.20 (dtd, J=7.5, 5.2, 2.5 Hz, 2H), 7.15-7.07 (m, 4H), 4.34 (q, J=5.3 Hz, 1H), 4.08 (q, J=5.2 Hz, 3H), 3.46-3.42 (m, 1H), 3.39 (q, J=3.3 Hz, 2H), 1.65-1.48 (m, 4H).

### Intermediate 3D: (1R,5S)-3-benzhydryl-8-azabicyclo[3.2.1]octane

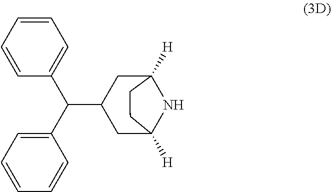 [SMILES: c1ccc(C(c2ccccc2)C2C[C@H]3CC[C@@H](C2)N3)cc1]

To a stirred solution of (1R,5S)-3-benzhydryl-8-azabicyclo[3.2.1]oct-2-ene (103 mg, 0.374 mmol) in EtOH (2.0 mL) at ambient temperature were added palladium on carbon (10% w/w, 40.0 mg, 0.0374 mmol), AcOH (21.4 μL, 0.374 mmol), and triethylsilane (181 μL, 1.12 mmol). The reaction mixture was stirred at 80° C. for 24 h. The reaction mixture was filtered through a glass fiber filter and washed with EtOAc. The filtrate was concentrated under reduced pressure. The reaction mixture was washed with a saturated aqueous solution of NaHCO₃, then brine, dried over Na₂SO₄, filtered, and concentrated under reduced pressure to afford (1R,5S)-3-benzhydryl-8-azabicyclo[3.2.1]octane as a white solid (87.0 mg, 97% p., 81% y.). m/z [M+H]⁺=278.4, ¹H NMR (400 MHz, DMSO-d₆): δ (ppm) 7.35 (ddd, J=8.0, 5.0, 1.4 Hz, 4H), 7.29-7.19 (m, 4H), 7.16-7.07 (m, 2H), 3.30 (td, J=13.9, 5.3 Hz, 5H), 2.71-2.52 (m, 1H), 1.63 (dd, J=23.0, 2.2 Hz, 4H), 1.27-1.17 (m, 1H), 1.13-0.95 (m, 2H).

### Intermediate 3E: tert-butyl(4S)-5-amino-4-[5-[(1R,5S)-3-benzhydryl-8-azabicyclo[3.2.1]octane-8-carbonyl]-1-oxo-isoindolin-2-yl]-5-oxo-pentanoate

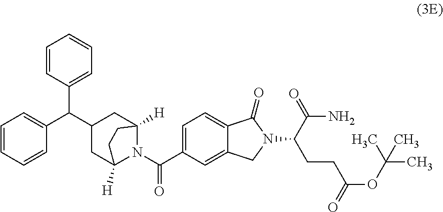 [SMILES: CC(C)(C)OC(=O)CC[C@@H](C(N)=O)N1Cc2cc(C(=O)N3[C@@H]4CC[C@H]3CC(C(c3ccccc3)c3ccccc3)C4)ccc2C1=O]

In a sealed vial under argon, to a stirred solution of (1R,5S)-3-benzhydryl-8-azabicyclo[3.2.1]octane (87.0 mg, 0.314 mmol), 2-[(1S)-4-tert-butoxy-1-carbamoyl-4-oxo-butyl]-1-oxo-isoindoline-5-carboxylic acid (116 mg, 0.314 mmol), and DIPEA (219 μL, 1.25 mmol) in EtOAc (1.0 mL) was added a solution of T3P in EtOAc (50% w/w, 299 mg, 0.470 mmol). The reaction mixture was stirred at ambient temperature for 70 h. The reaction was quenched with the addition of water. The aqueous layer was extracted with EtOAc (2×). The combined organic layers were washed with a 0.5 N aqueous solution of HCl, a saturated aqueous solution of NaHCO₃, brine, filtered through hydrophobic paper, and concentrated under reduced pressure. The crude was purified by flash chromatography on silica gel using a gradient of EtOAc/EtOH (3/1) in n-heptane from 0% to 70% to afford tert-butyl(4S)-5-amino-4-[5-[(1R,5S)-3-benzhydryl-8-azabicyclo[3.2.1]octane-8-carbonyl]-1-oxo-isoindolin-2-yl]-5-oxo-pentanoate as a white foam (100 mg, 99% p., 51% y.). m/z [M+H]⁺=622.6.

### Example 3

In a sealed vial, a solution of tert-butyl(4S)-5-amino-4-[5-[(1R,5S)-3-benzhydryl-8-azabicyclo[3.2.1]octane-8-carbonyl]-1-oxo-isoindolin-2-yl]-5-oxo-pentanoate (100 mg, 0.161 mmol) and 4-methylbenzenesulfonic acid hydrate (91.8 mg, 0.482 mmol) in acetonitrile (1.0 mL) was stirred at 70° C. for 16 h. The mixture was cooled to ambient temperature. The reaction was quenched with the addition of a saturated aqueous solution of NaHCO₃. The aqueous layer was extracted with EtOAc (2×). The combined organic layers were washed with brine, filtered through hydrophobic paper, and the filtrate was concentrated under reduced pressure. The crude was purified by flash chromatography on silica gel using a gradient of EtOAc/EtOH (3/1) in n-heptane from 0% to 100%. The desired fractions were concentrated under reduced pressure. The resulting solid was solubilized in the minimum amount of DCM and the homogenous solution was added dropwise to a stirred solution of n-pentane. The mixture was stirred for 1 h. The resulting precipitate was filtered, washed with n-pentane, and dried under reduced pressure at 50° C. overnight to afford (3S)-3-[5-[(1R,5S)-3-benzhydryl-8-azabicyclo[3.2.1]octane-8-carbonyl]-1-oxo-isoindolin-2-yl]piperidine-2,6-dione as a white solid (48.0 mg, 99.0% p., 53% y.). m/z [M+H]⁺=548.4, ¹H NMR (500 MHz, DMSO-d₆): δ (ppm) 10.99 (s, 1H), 7.77 (dd, J=7.75, 4.00 Hz, 1H), 7.69 (d, J=10.63 Hz, 1H), 7.63-7.50 (m, 1H), 7.49-7.35 (m, 4H), 7.26 (dt, J=13.70, 7.47 Hz, 4H), 7.18-6.96 (m, 2H), 5.13 (dd, J=13.26, 5.00 Hz, 1H), 4.71-4.54 (m, 1H), 4.53-4.31 (m, 2H), 3.59 (d, J=11.01 Hz, 2H), 3.00-2.90 (m, 1H) 2.89 (br d, J=5.50 Hz, 1H), 2.66-2.57 (m, 1H), 2.40 (br t, J=13.32 Hz, 1H), 2.14-2.02 (m, 1H), 2.01-1.66 (m, 5H), 1.46-1.16 (m, 3H).

### Example 4

### (3S)-3-[5-(7-benzhydryl-3-oxa-9-azabicyclo[3.3.1]nonane-9-carbonyl)-1-oxo-isoindolin-2-yl]piperidine-2,6-dione

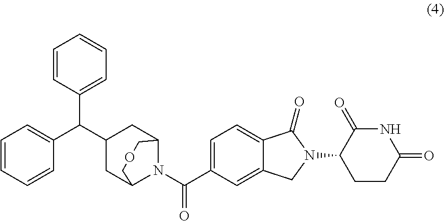 [SMILES: CC(=O)CC[C@@H](C(N)=O)N1Cc2cc(C(=O)N3C4COCC3CC(C(c3ccccc3)c3ccccc3)C4)ccc2C1=O]

### Intermediate 4A: tert-butyl 7-[hydroxy (diphenyl)methyl]-3-oxa-9-azabicyclo[3.3.1]nonane-9-carboxylate

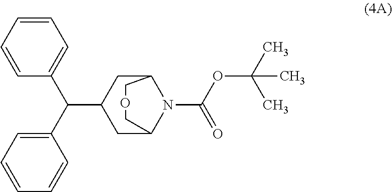 [SMILES: CC(C)(C)OC(=O)N1C2COCC1CC(C(c1ccccc1)c1ccccc1)C2]

In a three-necked round-bottom flask under nitrogen, to a stirred solution of 9-tert-butyl 7-methyl 3-oxa-9-azabicyclo[3.3.1]nonane-7,9-dicarboxylate (242 mg, 0.823 mmol) in dry THF (3.1 mL) at 0° C. under nitrogen was added a 3 M solution of bromo (phenyl) magnesium (603 μL, 1.81 mmol) in dry THF. The reaction mixture was stirred at ambient temperature overnight. The reaction was quenched with the addition of a saturated aqueous solution of NH₄Cl at 0° C. The aqueous layer was extracted with EtOAc (3×). The combined organic layers were washed with brine, dried using a phase separator cartridge and concentrated under reduced pressure to afford tert-butyl 7-[hydroxy (diphenyl)methyl]-3-oxa-9-azabicyclo[3.3.1]nonane-9-carboxylate as a pale yellow oil (349 mg, 45% p., 47% y.). m/z [M+H]⁺=336.3.

### Intermediate 4B: 7-benzhydryl-3-oxa-9-azabicyclo[3.3.1]nonane

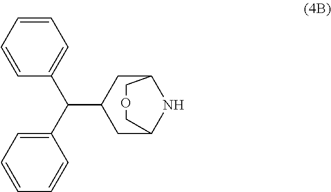 [SMILES: c1ccc(C(c2ccccc2)C2CC3COCC(C2)N3)cc1]

In a round-bottom flask, to a solution of tert-butyl 7-[hydroxy (diphenyl)methyl]-3-oxa-9-azabicyclo[3.3.1]nonane-9-carboxylate (348 mg, 0.382 mmol) in dry DCM (3.0 mL), was added triethylsilane (410 μL, 2.57 mmol). The reaction mixture was cooled to 0° C. and TFA (195 μL, 25.5 mmol) was added. The reaction mixture was stirred at ambient temperature overnight. The reaction mixture was basified with a saturated aqueous solution of NaHCO₃. The aqueous layer was extracted with DCM (3×). The combined organic layers were washed with brine, dried using a phase separator cartridge, and concentrated under reduced pressure to afford 7-benzhydryl-3-oxa-9-azabicyclo[3.3.1]nonane as a pale yellow solid (118 mg, 46% p., 48% y.). m/z [M+H]⁺=294.3, ¹H NMR (400 MHz, DMSO-d₆): δ (ppm) 9.31 (s, 1H), 7.34 (dd, J=8.1, 1.4 Hz, 3H), 7.27 (s, 2H), 7.15 (tdd, J=8.7, 7.0, 1.9 Hz, 3H), 6.77-6.72 (m, 2H), 3.74-3.67 (m, 4H), 3.43-3.37 (m, 2H), 2.89-2.79 (m, 2H), 1.64-1.52 (m, 2H), 1.35 (dddd, J=14.0, 11.5, 7.0, 3.6 Hz, 2H).

### Intermediate 4C: tert-butyl(4S)-5-amino-4-[5-(7-benzhydryl-3-oxa-9-azabicyclo[3.3.1]nonane-9-carbonyl)-1-oxo-isoindolin-2-yl]-5-oxo-pentanoate

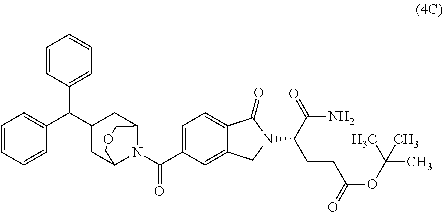 [SMILES: CC(C)(C)OC(=O)CC[C@@H](C(N)=O)N1Cc2cc(C(=O)N3C4COCC3CC(C(c3ccccc3)c3ccccc3)C4)ccc2C1=O]

To a stirred solution of 2-[(1S)-4-tert-butoxy-1-carbamoyl-4-oxo-butyl]-1-oxo-isoindoline-5-carboxylic acid (156 mg, 0.430 mmol) in EtOAc (2.0 mL) were added successively 7-benzhydryl-3-oxa-9-azabicyclo[3.3.1]nonane (115 mg, 0.180 mmol), DIPEA (210 μL, 1.20 mmol) and a solution of T3P in MeTHF (50% w/w, 374 mg, 0.588 mmol). The reaction mixture was stirred at ambient temperature for 2 h. Additional solution of T3P in MeTHF (50% w/w, 374 mg, 0.588 mmol) was added and the reaction mixture was stirred at ambient temperature for 2 h. Additional solution of T3P in MeTHF (50% w/w, 374 mg, 0.588 mmol) was added and the reaction mixture was stirred at ambient temperature overnight. The reaction was quenched with the addition of water. The aqueous layer was extracted with EtOAc (3×). The combined organic layers were washed with brine, dried using a phase separator cartridge and concentrated under reduced pressure. The crude was purified by flash chromatography on silica gel using a gradient of EtOAc/EtOH (3/1) in cyclohexane from 5% to 60% to afford tert-butyl(4S)-5-amino-4-[5-(7-benzhydryl-3-oxa-9-azabicyclo[3.3.1]nonane-9-carbonyl)-1-oxo-isoindolin-2-yl]-5-oxo-pentanoate as a pale yellow solid (20.2 mg, 94% p., 35% y). m/z [M+H]⁺=638.5, ¹H NMR (400 MHz, DMSO-d₆): δ (ppm) 7.74 (d, J=7.8 Hz, 1H), 7.65 (s, 1H), 7.56 (s, 1H), 7.51 (d, J=7.8 Hz, 1H), 7.41-7.21 (m, 8H), 7.14 (dt, J=19.4, 8.0 Hz, 3H), 4.77-4.70 (m, 1H), 4.63 (d, J=17.8 Hz, 1H), 4.49 (dd, J=17.9, 3.6 Hz, 1H), 4.42 (s, 1H), 3.88 (d, J=11.4 Hz, 1H), 3.73-3.61 (m, 3H), 3.53 (s, 2H), 3.43 (d, J=11.2 Hz, 1H), 2.16 (d, J=3.0 Hz, 3H), 1.99 (s, 2H), 1.69 (d, J=13.5 Hz, 1H), 1.50 (dd, J=27.0, 11.6 Hz, 2H), 1.31 (d, J=3.3 Hz, 9H).

### Example 4

To a stirred solution of tert-butyl(4S)-5-amino-4-[5-(7-benzhydryl-3-oxa-9-azabicyclo[3.3.1]nonane-9-carbonyl)-1-oxo-isoindolin-2-yl]-5-oxo-pentanoate (20.2 mg, 0.021 mmol) in acetonitrile (170 μL) was added 4-methylbenzenesulfonic acid hydrate (8.6 mg, 0.045 mmol). The reaction mixture was stirred at 80° C. for 3 h. The reaction mixture was cooled to ambient temperature and diluted with water. The aqueous layer was extracted with EtOAc (3×). The combined organic layers were washed with brine, dried over Na₂SO₄, filtered, and concentrated under reduced pressure. The crude was purified by flash chromatography on silica gel using a gradient of a EtOAc/EtOH (3/1) in cyclohexane from 10% to 100%. The residue was solubilized in a minimum amount of acetone and the homogenous solution was added to n-pentane dropwise. The resulting precipitate was triturated for 1 h, filtered, washed with n-pentane and dried under reduced pressure at 50° C. overnight to afford (3S)-3-[5-(7-benzhydryl-3-oxa-9-azabicyclo[3.3.1]nonane-9-carbonyl)-1-oxo-isoindolin-2-yl]piperidine-2,6-dione as a white powder (3.3 mg, 98.3% p., 27% y.). m/z [M+H]⁺=564.3, ¹H NMR (500 MHz, DMSO-d₆): δ (ppm) 10.99 (s, 1H), 7.78 (d, J=7.8 Hz, 1H), 7.67 (s, 1H), 7.55 (d, J=7.8 Hz, 1H), 7.4-7.3 (m, 4H), 7.3-7.2 (m, 4H), 7.2-7.1 (m, 2H), 5.12 (td, J=13.2, 4.7 Hz, 1H), 4.6-4.3 (m, 3H), 3.9-3.4 (m, 7H), 3.0-2.8 (m, 1H), 2.7-2.6 (m, 1H), 2.5-2.3 (m, 1H), 2.1-1.9 (m, 1H), 1.8-1.6 (m, 1H), 1.6-1.3 (m, 3H).

### Example 5

### (3S)-3-[5-[4-[(2-chlorophenyl)-phenyl-methyl]piperidine-1-carbonyl]-6-methyl-1-oxo-isoindolin-2-yl]piperidine-2,6-dione

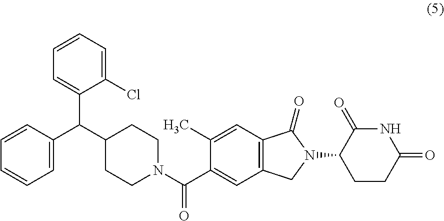 [SMILES: Cc1cc2c(cc1C(=O)N1CCC(C(c3ccccc3)c3ccccc3Cl)CC1)CN([C@H]1CCC(=O)NC1=O)C2=O]

### Intermediate 5A: tert-butyl 4-[(2-chlorophenyl)-hydroxy-phenyl-methyl]piperidine-1-carboxylate

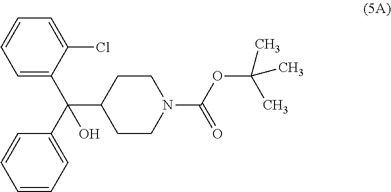 [SMILES: CC(C)(C)OC(=O)N1CCC(C(O)(c2ccccc2)c2ccccc2Cl)CC1]

To a stirred solution of 1-bromo-2-chloro-benzene (157 μL, 1.33 mmol) in dry THF (1.0 mL) at −78° C. under argon was added a 1.6 M solution of n-butyllithium in hexanes (829 μL, 1.33 mmol). The mixture was stirred at −78° C. for 30 min. A solution of tert-butyl 4-benzoylpiperidine-1-carboxylate (200 mg, 0.663 mmol) in dry THF (1.0 mL) was added and the reaction mixture was stirred for 1 h at −78° C. The reaction was quenched with the addition of a saturated aqueous solution of NH₄Cl. The aqueous layer was extracted with EtOAc (3×). The combined organic layers were washed with brine, dried over Na₂SO₄, filtered, and concentrated under reduced pressure. The crude was purified by flash chromatography on silica gel using a gradient of EtOAc in n-heptane from 1% to 50%. Fractions were combined and concentrated under reduced pressure to afford tert-butyl 4-[(2-chlorophenyl)-hydroxy-phenyl-methyl]piperidine-1-carboxylate as a white foam (221 mg, 94% p., 78% y.). m/z [M−Boc+2H]⁺=302.3, ¹H NMR (400 MHz, DMSO-d₆): δ (ppm) 8.03-7.99 (m, 1H), 7.41-7.34 (m, 3H), 7.28-7.20 (m, 4H), 7.18-7.13 (m, 1H), 5.48 (s, 1H), 3.95 (s, 2H), 3.11 (s, 1H), 2.71 (d, J=35.6 Hz, 2H), 1.52 (s, 1H), 1.37 (s, 9H), 1.25 (ddd, J=37.0, 14.4, 8.0 Hz, 3H).

### Intermediate 5B: 4-[(2-chlorophenyl)-phenyl-methyl]piperidine

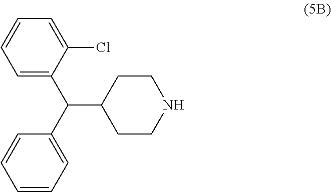 [SMILES: Clc1ccccc1C(c1ccccc1)C1CCNCC1]

In a sealed vial, to a stirred solution of tert-butyl 4-[(2-chlorophenyl)-hydroxy-phenyl-methyl]piperidine-1-carboxylate (220 mg, 0.515 mmol) and triethylsilane (410 μL, 2.57 mmol) in dry DCM (1.7 mL) was added dropwise TFA (118 μL, 15.4 mmol) at 0° C. The reaction mixture was allowed to warm to ambient temperature and was stirred at ambient temperature overnight. The reaction mixture was poured into a mixture of ice and an aqueous solution of NaOH (33% w/w). The aqueous layer was extracted with DCM (2×). The combined organic layers were dried over Na₂SO₄, filtered, and concentrated under reduced pressure to afford 4-[(2-chlorophenyl)-phenyl-methyl]piperidine as a colorless oil (200 mg, 70% p. 95% y.). m/z [M+H]⁺=286.1, ¹H NMR (400 MHz, DMSO-d₆): δ (ppm) 7.67 (dd, J=7.8, 1.7 Hz, 1H), 7.52-7.09 (m, 10H), 4.09 (d, J=11.1 Hz, 1H), 2.88-2.65 (m, 3H), 2.46-2.15 (m, 4H), 1.34 (dd, J=21.7, 13.1 Hz, 1H).

### Intermediate 5C: tert-butyl(4S)-5-amino-4-[5-[4-[(2-chlorophenyl)-phenyl-methyl]piperidine-1-carbonyl]-6-methyl-1-oxo-isoindolin-2-yl]-5-oxo-pentanoate

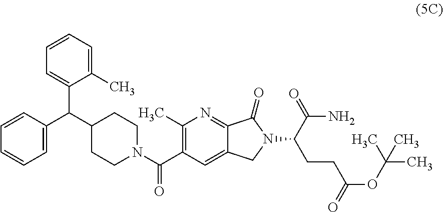 [SMILES: Cc1ccccc1C(c1ccccc1)C1CCN(C(=O)c2cc3c(nc2C)C(=O)N([C@@H](CCC(=O)OC(C)(C)C)C(N)=O)C3)CC1]

In a sealed vial under argon, a solution of 4-[(2-chlorophenyl)-phenyl-methyl]piperidine (75.0 mg, 0.184 mmol), 2-[(1S)-4-tert-butoxy-1-carbamoyl-4-oxo-butyl]-6-methyl-1-oxo-isoindoline-5-carboxylic acid (87.3 mg, 0.220 mmol) and DIPEA (128 μL, 0.735 mmol) in EtOAc (1.8 mL) was stirred at ambient temperature. A solution of T3P in EtOAc (50% w/w, 164 μL, 0.276 mmol) was added and the mixture was stirred at ambient temperature for 16 h. A saturated aqueous solution of NaHCO₃ was added and the aqueous layer was extracted with EtOAc (2X). The combined organic layers were washed with brine, dried over Na₂SO₄, and concentrated under reduced pressure. The crude was purified by flash chromatography on silica gel using a gradient of EtOAc/EtOH (3/1) in n-heptane from 1% to 70%. Fractions were combined and concentrated under reduced pressure to afford tert-butyl(4S)-5-amino-4-[5-[4-[(2-chlorophenyl)-phenyl-methyl]piperidine-1-carbonyl]-6-methyl-1-oxo-isoindolin-2-yl]-5-oxo-pentanoate as a white foam (41.0 mg, 98% p., 34% y.). m/z [M+H]⁺=644.3, ¹H NMR (400 MHz, DMSO-d₆).

### Example 5

In a sealed vial, a solution of tert-butyl(4S)-5-amino-4-[5-[4-[(2-chlorophenyl)-phenyl-methyl]piperidine-1-carbonyl]-6-methyl-1-oxo-isoindolin-2-yl]-5-oxo-pentanoate (39.0 mg, 0.060 mmol) and 4-methylbenzenesulfonic acid hydrate (34.5 mg, 0.182 mmol) in acetonitrile (390 μL) was stirred at 70° C. for 16 h. The mixture was cooled to ambient temperature. The reaction was quenched with the addition of a saturated aqueous NaHCO₃. The aqueous layer was extracted with EtOAc (2×). The combined organic layers were washed with brine. Layers were separated using hydrophobic paper and the filtrate was concentrated under reduced. The crude was purified by flash chromatography on silica gel using a gradient of EtOAc/EtOH (3/1) in n-heptane from 0% to 100%. The desired fractions were concentrated. The resulting gum was solubilized in a minimum amount of DCM and the solution was added dropwise to stirred solution of n-pentane. The mixture was stirred for 1 h. The resulting precipitate was filtered, washed with n-pentane and dried under reduced pressure at 50° C. for 16 h to afford (3S)-3-[5-[4-[(2-chlorophenyl)-phenyl-methyl]piperidine-1-carbonyl]-6-methyl-1-oxo-isoindolin-2-yl]piperidine-2,6-dione as a white solid (13.5 mg, 97.6% p, 38% y.). m/z [M+H]⁺=570.4, ¹H NMR (600 MHz, DMSO-d₆): δ (ppm) 10.97 (s, 1H), 7.78-7.66 (m, 1H), 7.62-7.59 (m, 1H), 7.50-7.27 (m, 6H), 7.26-7.11 (m, 3H), 5.16-5.04 (m, 1H), 4.52 (br s, 1H), 4.46-4.37 (m, 1H), 4.33-4.27 (m, 1H), 4.17-4.12 (m, 1H), 3.28-3.17 (m, 1H), 3.03-2.85 (m, 2H), 2.81-2.71 (m, 1H), 2.65-2.53 (m, 2H), 2.42-2.35 (m, 1H), 2.35-2.20 (m, 3H), 1.98 (br s, 1H), 1.70-0.89 (m, 4H).

### Example 6

### (3S)-3-[5-[(1R,4R)-5-benzhydryl-2-azabicyclo[2.2.1]heptane-2-carbonyl]-1-oxo-isoindolin-2-yl]piperidine-2,6-dione

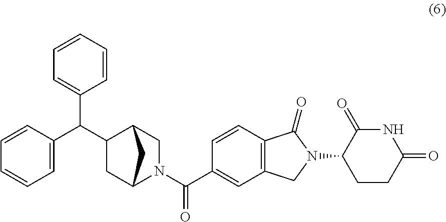 [SMILES: O=C1CC[C@H](N2Cc3cc(C(=O)N4C[C@@H]5C[C@H]4CC5C(c4ccccc4)c4ccccc4)ccc3C2=O)C(=O)N1]

### Intermediate 6A: tert-butyl(1S,4S)-5-benzhydryl-5-hydroxy-2-azabicyclo[2.2.1]heptane-2-carboxylate

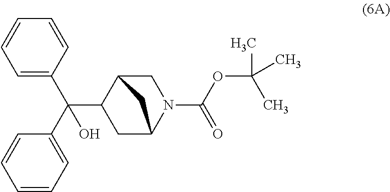 [SMILES: CC(C)(C)OC(=O)N1C[C@@H]2C[C@H]1CC2C(O)(c1ccccc1)c1ccccc1]

To a stirred solution of benzhydryl bromide (715 mg, 2.60 mmol) and (1S,4S)-2-Boc-5-oxo-2-azabicyclo[2.2.1]heptane (500 mg, 2.37 mmol) in dry THF (12 mL) at −78° C. under nitrogen was added a 2.5 M solution of n-butyllithium in hexanes (2.1 mL, 5.21 mmol) dropwise. The reaction mixture was stirred for 1 h at −78° C. The reaction was quenched with the addition of a saturated aqueous solution of NH₄Cl. The reaction mixture was allowed to warm up to ambient temperature and the aqueous layer was extracted with EtOAc (3×). The combined organic layers were washed with brine, dried over Na₂SO₄, filtered, and concentrated under reduced pressure. The crude was purified by flash chromatography on silica gel using a gradient of EtOAc in cyclohexane from 1% to 50% to afford tert-butyl(1S,4S)-5-benzhydryl-5-hydroxy-2-azabicyclo[2.2.1]heptane-2-carboxylate as a white foam (182 mg, 99% p., 20% y.). m/z [M+H]⁺=380.3, ¹H NMR (400 MHz, DMSO-d₆): δ (ppm) 7.60-7.55 (m, 2H), 7.46 (d, J=7.5 Hz, 2H), 7.24 (t, J=7.4 Hz, 4H), 7.19-7.10 (m, 2H), 4.90 (d, J=6.2 Hz, 1H), 3.97 (d, J=24.2 Hz, 2H), 3.55 (dd, J=14.3, 9.5 Hz, 1H), 2.89 (t, J=10.3 Hz, 1H), 2.24 (s, 1H), 1.97 (d, J=16.2 Hz, 1H), 1.71-1.48 (m, 2H), 1.36 (m, 10H).

### Intermediate 6B: (1R,4R)-5-benzhydryl-2-azabicyclo[2.2.1]heptane

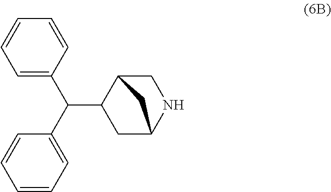 [SMILES: c1ccc(C(c2ccccc2)C2C[C@@H]3C[C@H]2CN3)cc1]

To a stirred solution of tert-butyl(1S,4S)-5-benzhydryl-5-hydroxy-2-azabicyclo[2.2.1]heptane-2-carboxylate (180 mg, 0.474 mmol) in THF (1.6 mL) was added sulfuric acid (758 μL, 14.2 mmol). The reaction mixture was stirred at 65° C. for 24 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. In a sealed vial, to a stirred solution of the crude in EtOH (1.6 mL) was added palladium on carbon (10% w/w, 50.5 mg, 0.0474 mmol). The reaction mixture was degassed with argon for 5 min and triethylsilane (383 μL, 2.37 mmol) was added. The reaction mixture was stirred at ambient temperature overnight. Additional palladium on carbon (10% w/w, 50.5 mg, 0.047 mmol) and triethylsilane (383 μL, 2.37 mmol) were added and the reaction mixture was stirred for 24 h. The reaction mixture was filtered through a glass fiber filter and the filtrate was concentrated under reduced pressure to afford (1R,4R)-5-benzhydryl-2-azabicyclo[2.2.1]heptane as a yellow oil (868 mg, 65% p., quant. yield). m/z [M+H]⁺=264.4.

### Intermediate 6C: tert-butyl(4S)-5-amino-4-[5-[(1R,4R)-5-benzhydryl-2-azabicyclo[2.2.1]heptane-2-carbonyl]-1-oxo-isoindolin-2-yl]-5-oxo-pentanoate

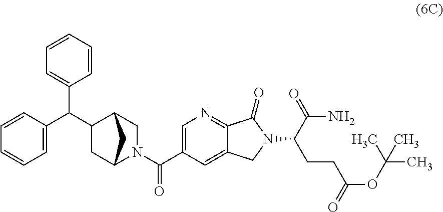 [SMILES: CC(C)(C)OC(=O)CC[C@@H](C(N)=O)N1Cc2cc(C(=O)N3C[C@@H]4C[C@H]3CC4C(c3ccccc3)c3ccccc3)cnc2C1=O]

To a stirred suspension of 2-[(1S)-4-tert-butoxy-1-carbamoyl-4-oxo-butyl]-1-oxo-isoindoline-5-carboxylic acid (125 mg, 0.345 mmol) in EtOAc (3.4 mL) were added successively (1R,4R)-5-benzhydryl-2-azabicyclo[2.2.1]heptane (99.9 mg, 0.379 mmol), DIPEA (181 μL, 1.03 mmol), and then a solution of T3P in MeTHF (50% w/w, 329 mg, 0.517 mmol). The reaction mixture was stirred at ambient temperature overnight. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by flash chromatography on silica gel using a gradient of EtOAc/EtOH (3/1) in cyclohexane from 2% to 100% to afford tert-butyl(4S)-5-amino-4-[5-[(1R,4R)-5-benzhydryl-2-azabicyclo[2.2.1]heptane-2-carbonyl]-1-oxo-isoindolin-2-yl]-5-oxo-pentanoate as an off-white solid (143 mg, 99% p., 68% y.). m/z [M+H]⁺=608.7.

### Example 6

To a stirred solution of tert-butyl(4S)-5-amino-4-[5-[(1R,4R)-5-benzhydryl-2-azabicyclo[2.2.1]heptane-2-carbonyl]-1-oxo-isoindolin-2-yl]-5-oxo-pentanoate (140 mg, 0.230 mmol) in acetonitrile (2.3 mL) was added 4-methylbenzenesulfonic acid hydrate (131 mg, 0.691 mmol). The reaction mixture was stirred at 80° C. overnight. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by flash chromatography on silica gel using a gradient of EtOAc/EtOH (3/1) in cyclohexane from 10% to 100%. The residue was solubilized in the minimum amount of DCM and the homogenous solution was added to a solution of n-pentane dropwise. The resulting precipitate was triturated for 1 h, filtered, washed with pentane, and dried under reduced pressure at 50° C. overnight to afford (3S)-3-[5-[(1R,4R)-5-benzhydryl-2-azabicyclo[2.2.1]heptane-2-carbonyl]-1-oxo-isoindolin-2-yl]piperidine-2,6-dione as a white solid (52.5 mg, 99.2% p., 42% y.). m/z [M+H]⁺=534.3, ¹H NMR (500 MHz, DMSO-d₆): δ (ppm) 10.99 (br s, 1H), 7.79-7.74 (m, 1H), 7.65 (s, 1H), 7.54 (ddd, J=7.8, 2.9, 1.3 Hz, 1H), 7.51-7.05 (m, 10H), 5.22-5.05 (m, 1H), 4.57-4.43 (m, 1H), 4.43-4.32 (m, 1H), 3.99-3.94 (m, 1H), 3.76-3.53 (m, 1H), 3.47-3.34 (m, 2H), 3.30-3.04 (m, 2H), 2.97-2.85 (m, 1H), 2.67-2.56 (m, 1H), 2.44-2.33 (m, 1H), 2.28-2.05 (m, 1H), 2.04-1.97 (m, 1H), 1.91-1.45 (m, 2H), 1.24-1.11 (m, 1H).

### Example 7

### (3S)-3-[5-[(1R,5S)-3-benzhydryl-8-azabicyclo[3.2.1]octane-8-carbonyl]-6-fluoro-1-oxo-isoindolin-2-yl]piperidine-2,6-dione

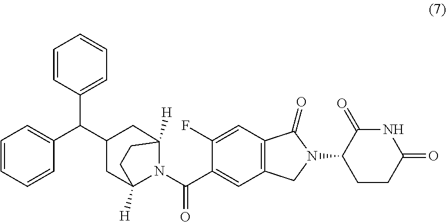 [SMILES: O=C1CC[C@H](N2Cc3cc(C(=O)N4[C@@H]5CC[C@H]4CC(C(c4ccccc4)c4ccccc4)C5)c(F)cc3C2=O)C(=O)N1]

### Intermediate 7A: (1R,5S)-3-benzhydrylidene-8-azabicyclo[3.2.1]octane

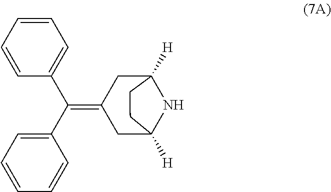 [SMILES: c1ccc(C(=C2C[C@H]3CC[C@@H](C2)N3)c2ccccc2)cc1]

In a sealed vial under argon, sulfuric acid (3.7 mL, 70.1 mmol) was added to a stirred solution of tert-butyl(1R,5S)-3-benzhydryl-3-hydroxy-8-azabicyclo[3.2.1]octane-8-carboxylate (968.4 mg, 2.34 mmol) in THF (4.5 mL). The mixture was stirred at 65° C. for 16 h. The reaction was quenched with the addition of a saturated aqueous solution of Na₂CO₃. The aqueous layer was extracted with DCM (3×). The combined organic layers were washed with brine, dried over Na₂SO₄, filtered, and concentrated under reduced pressure to afford (1R,5S)-3-benzhydrylidene-8-azabicyclo[3.2.1]octane as an orange oil (705 mg, 89% p., 97% y.). m/z [M+H]⁺=276.2, ¹H NMR (400 MHz, DMSO-d₆): δ (ppm) 7.36-7.28 (m, 4H), 7.25-7.20 (m, 2H), 7.19-7.12 (m, 4H), 3.75-3.65 (m, 2H), 3.36-3.30 (m, 2H), 2.37 (dd, J=15.2, 3.1 Hz, 1H), 2.24 (dt, J=14.6, 1.9 Hz, 1H), 1.86-1.76 (m, 2H), 1.68 (q, J=6.5 Hz, 1H), 1.54-1.39 (m, 2H).

### Intermediate 7B: (1S,5R)-3-benzhydryl-8-azabicyclo[3.2.1]octane

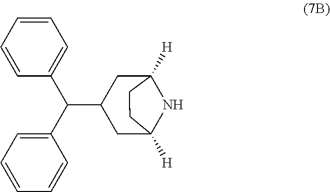 [SMILES: c1ccc(C(c2ccccc2)C2C[C@H]3CC[C@@H](C2)N3)cc1]

In a sealed vial, to a stirred solution of (1S,5R)-3-benzhydrylidene-8-azabicyclo[3.2.1]octane (705 mg, 2.28 mmol) in EtOH (4.5 mL) at ambient temperature were added palladium on carbon (10% w/w, 242 mg, 0.228 mmol) and AcOH (130 μL, 2.28 mmol). The suspension was degassed with argon for 5 min. Triethylsilane (1.1 mL, 6.84 mmol) was added and the reaction mixture was stirred at 80° C. for 16 h. Additional palladium on carbon (10% w/w, 242 mg, 0.228 mmol), AcOH (130 μL, 2.28 mmol) and triethylsilane (1.1 mL, 6.84 mmol) were added and the reaction mixture was stirred at ambient temperature for 10 min and at 80° C. for 23 h. Additional AcOH (130 μL, 2.28 mmol), palladium on carbon (10% w/w, 242 mg, 0.228 mmol), and triethylsilane (1.1 mL, 6.84 mmol) were added and the reaction mixture was stirred at ambient temperature for 10 min, at 80° C. for 5.5 h and at ambient temperature for 4 days. Additional palladium on carbon (10% w/w, 242 mg, 0.228 mmol), AcOH (130 μL, 2.28 mmol) and triethylsilane (1.1 mL, 6.84 mmol) were added and the reaction mixture was stirred at ambient temperature for 10 min and at 80° C. for 23 h. The reaction mixture was filtered through a pad of talc, washed with EtOH and the filtrate was concentrated under reduced pressure. The resulting residue was triturated in n-pentane, filtered, and dried under reduced pressure to afford (1S,5R)-3-benzhydryl-8-azabicyclo[3.2.1]octane as a white solid (201 mg, 93% p., 30% y.). m/z [M+H]⁺=278.3, ¹H NMR (400 MHz, DMSO-d₆): δ 8.64 (s, 1H), 7.43 (td, J=8.3, 1.3 Hz, 4H), 7.28 (td, J=7.6, 3.3 Hz, 4H), 7.21-7.11 (m, 2H), 3.84 (d, J=12.0 Hz, 2H), 2.95-2.76 (m, 1H), 2.11-1.99 (m, 3H), 1.91 (d, J=2.2 Hz, 2H), 1.51 (p, J=2.8 Hz, 1H), 1.43-1.32 (m, 3H).

### Intermediate 7C: tert-butyl(4S)-5-amino-4-[5-[(1R,5S)-3-benzhydryl-8-azabicyclo[3.2.1]octane-8-carbonyl]-6-fluoro-1-oxo-isoindolin-2-yl]-5-oxo-pentanoate

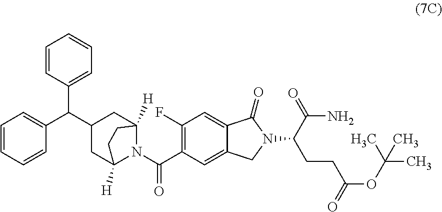 [SMILES: CC(C)(C)OC(=O)CC[C@@H](C(N)=O)N1Cc2cc(C(=O)N3[C@@H]4CC[C@H]3CC(C(c3ccccc3)c3ccccc3)C4)c(F)cc2C1=O]

In a sealed vial under argon, a solution of (1R,5S)-3-benzhydryl-8-azabicyclo[3.2.1]octane (70.0 mg, 0.25 mmol), 2-[(1S)-4-tert-butoxy-1-carbamoyl-4-oxo-butyl]-6-fluoro-1-oxo-isoindoline-5-carboxylic acid (120 mg, 0.303 mmol) and DIPEA (176 μL, 1.01 mmol) in EtOAc (2.5 mL) was stirred at ambient temperature. A solution of T3P in EtOAc (50% w/w, 225 μL, 0.379 mmol) was added and the mixture was stirred at ambient temperature for 15 h. Additional DIPEA (176 μL, 1.01 mmol) and a solution of T3P in EtOAc (50% w/w, 225 μL, 0.379 mmol) were added and the reaction mixture was stirred at 60° C. for 3 h. A saturated aqueous solution of NaHCO₃ was added and the aqueous layer was extracted with EtOAc (2×). The combined organic layers were washed with brine, dried over Na₂SO₄ and concentrated. The crude was purified by flash chromatography on silica gel using a gradient of EtOAc/EtOH (3/1) in n-heptane from 1% to 100%. Fractions were combined and concentrated under reduced pressure to afford tert-butyl(4S)-5-amino-4-[5-[(1R,5S)-3-benzhydryl-8-azabicyclo[3.2.1]octane-8-carbonyl]-6-fluoro-1-oxo-isoindolin-2-yl]-5-oxo-pentanoate as an off-white foam (75.6 mg, 99% p., 46% y.). m/z [M+H]⁺=640.5, ¹H NMR (400 MHz, DMSO-d₆): δ (ppm) 7.75-7.65 (m, 1H), 7.57 (dd, J=8.4, 6.6 Hz, 2H), 7.47-7.35 (m, 4H), 7.32-7.20 (m, 5H), 7.19-7.13 (m, 1H), 7.13-7.06 (m, 1H), 4.73 (dd, J=10.3, 4.7 Hz, 1H), 4.59 (td, J=17.6, 17.5, 7.4 Hz, 2H), 4.47 (dd, J=17.7, 7.3 Hz, 1H), 3.91 (dd, J=12.3, 6.6 Hz, 1H), 3.71-3.59 (m, 1H), 2.96-2.58 (m, 1H), 2.17 (q, J=8.4, 6.8, 6.8 Hz, 3H), 2.00 (d, J=11.0 Hz, 3H), 1.89-1.74 (m, 3H), 1.37-1.21 (m, 11H), 1.09 (d, J=13.7 Hz, 1H).

### Example 7

In a sealed vial, a solution of tert-butyl(4S)-5-amino-4-[5-[(1R,5S)-3-benzhydryl-8-azabicyclo[3.2.1]octane-8-carbonyl]-6-fluoro-1-oxo-isoindolin-2-yl]-5-oxo-pentanoate (72.0 mg, 0.113 mmol) and 4-methylbenzenesulfonic acid hydrate (64.0 mg, 0.338 mmol) in acetonitrile (720 μL) was stirred at 70° C. for 16 h. The mixture was cooled to ambient temperature, a saturated aqueous solution of NaHCO₃ was added, and the aqueous layer was extracted with EtOAc (2×). The combined organic layers were washed with brine, filtered through hydrophobic paper, and concentrated under reduced pressure. The crude was purified by flash chromatography on silica gel using a gradient of EtOAc/EtOH (3/1) in n-heptane from 0% to 100%. The desired fractions were concentrated under reduced pressure. The residue was solubilized in a minimum amount of DCM and the solution was added dropwise to a stirred solution of n-pentane. The precipitate was stirred at ambient temperature for 1 h, filtered, washed with n-pentane, and dried under reduced pressure at 50° C. for 16 h to afford (3S)-3-[5-[(1R,5S)-3-benzhydryl-8-azabicyclo[3.2.1]octane-8-carbonyl]-6-fluoro-1-oxo-isoindolin-2-yl]piperidine-2,6-dione as a white solid (31.9 mg, 99.7% p., 49% y.). m/z [M+H]⁺=566.4, ¹H NMR (500 MHz, DMSO-d₆): δ (ppm) 11.01 (s, 1H), 7.76-7.67 (m, 1H), 7.62 (t, J=7.4 Hz, 1H), 7.45-7.37 (m, 4H), 7.31-7.21 (m, 4H), 7.18-7.09 (m, 2H), 5.17-5.08 (m, 1H), 4.65-4.52 (m, 1H), 4.52-4.44 (m, 1H), 4.40-4.33 (m, 1H), 3.92 (dd, J=12.3, 5.8 Hz, 0.55H), 3.72-3.65 (m, 0.45H), 3.54 (d, J=11.3 Hz, 1H), 2.95-2.87 (m, 1H), 2.70-2.57 (m, 2H), 2.46-2.35 (m, 1H), 2.09-1.71 (m, 6H), 1.47-1.22 (m, 2H), 1.16-1.08 (m, 1H).

### Example 8

### (3S)-3-[5-[(1R,5S)-3-benzhydryl-8-azabicyclo[3.2.1]octane-8-carbonyl]-4-methyl-1-oxo-isoindolin-2-yl]piperidine-2,6-dione

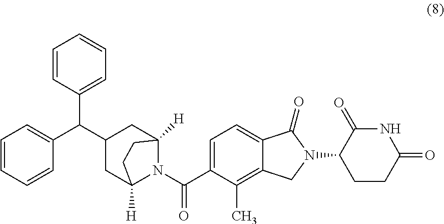 [SMILES: Cc1c(C(=O)N2[C@@H]3CC[C@H]2CC(C(c2ccccc2)c2ccccc2)C3)ccc2c1CN([C@H]1CCC(=O)NC1=O)C2=O]

### Intermediate 8A: tert-butyl(4S)-5-amino-4-[5-[(1R,5S)-3-benzhydryl-8-azabicyclo[3.2.1]octane-8-carbonyl]-4-methyl-1-oxo-isoindolin-2-yl]-5-oxo-pentanoate

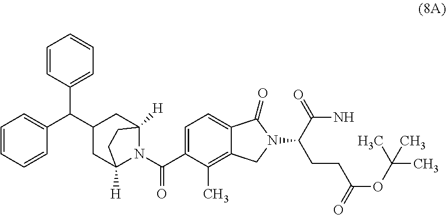 [SMILES: Cc1c(C(=O)N2[C@@H]3CC[C@H]2CC(C(c2ccccc2)c2ccccc2)C3)ccc2c1CN([C@@H](CCC(=O)OC(C)(C)C)C(N)=O)C2=O]

To a stirred solution of 2-[(1S)-4-tert-butoxy-1-carbamoyl-4-oxo-butyl]-4-methyl-1-oxo-isoindoline-5-carboxylic acid (70.0 mg, 0.177 mmol) in dry DMF (1.7 mL) under argon were added successively HATU (80.6 mg, 0.212 mmol) and DIPEA (154 μL, 0.883 mmol). The mixture was stirred for 45 min at ambient temperature. (1R,5S)-3-benzhydryl-8-azabicyclo[3.2.1]octane (63.2 mg, 0.212 mmol) was added and the solution was stirred at ambient temperature for 1 h. The reaction was quenched with the addition of water. EtOAc was added, followed by the addition of FastWorX® powder. EtOAc was removed under reduced pressure. The resin was filtered and washed with water. The resin was triturated in EtOAc, filtered, and the filtrate was concentrated under reduced pressure to afford tert-butyl(4S)-5-amino-4-[5-[(1R,5S)-3-benzhydryl-8-azabicyclo[3.2.1]octane-8-carbonyl]-4-methyl-1-oxo-isoindolin-2-yl]-5-oxo-pentanoate as a white solid (95.0 mg, 96% p., 81% y.). m/z [M+H]⁺=636.6, ¹H NMR (400 MHz, DMSO-d₆): δ (ppm) 7.58-7.53 (m, 2H), 7.45-7.41 (m, 2H), 7.38 (d, J=7.5 Hz, 3H), 7.32-7.27 (m, 2H), 7.27-7.21 (m, 2H), 7.21-7.05 (m, 3H), 4.77-4.71 (m, 1H), 4.63-4.51 (m, 2H), 4.46 (dd, J=17.6, 6.9 Hz, 1H), 3.93 (d, J=12.6 Hz, 1H), 3.59-3.45 (m, 1H), 2.69 (s, 1H), 2.26 (s, 3H), 2.17 (s, 3H), 2.04 (dd, J=14.5, 7.3 Hz, 1H), 1.93-1.65 (m, 4H), 1.40 (d, J=15.2 Hz, 1H), 1.35 (d, J=3.1 Hz, 1H), 1.31 (d, J=2.6 Hz, 9H), 1.24 (d, J=5.3 Hz, 1H), 1.07 (d, J=14.3 Hz, 1H).

### Example 8

A sealed vial was successively charged with tert-butyl(4S)-5-amino-4-[5-[(1R,5S)-3-benzhydryl-8-azabicyclo[3.2.1]octane-8-carbonyl]-4-methyl-1-oxo-isoindolin-2-yl]-5-oxo-pentanoate (93.7 mg, 0.147 mmol) and 4-methylbenzenesulfonic acid hydrate (84.1 mg, 0.442 mmol) in acetonitrile (737 μL). The reaction mixture was stirred at reflux overnight. The reaction was quenched with the addition of water. The aqueous layer was extracted with EtOAc (3×). The combined organic layers were washed with brine, dried over Na₂SO₄, filtered, and concentrated under reduced pressure. The crude was purified by flash chromatography on silica gel using a gradient of EtOAc/EtOH (3/1) in cyclohexane from 2% to 80%. The crude was solubilized in minimum amount of DCM and the homogeneous solution was added dropwise to a stirred solution of n-pentane. The resulting precipitate was filtered and dried under reduced pressure at 50° C. overnight to afford (3S)-3-[5-[(1R,5S)-3-benzhydryl-8-azabicyclo[3.2.1]octane-8-carbonyl]-4-methyl-1-oxo-isoindolin-2-yl]piperidine-2,6-dione as a white powder (32.0 mg, 96.8% p., 37% y.). m/z [M+H]⁺=562.4, ¹H NMR (500 MHz, DMSO-d₆): δ (ppm) 11.00 (s, 1H), 7.7-7.5 (m, 1H), 7.5-7.3 (m, 5H), 7.3-7.2 (m, 4H), 7.2-7.0 (m, 2H), 5.2-5.1 (m, 1H), 4.7-4.5 (m, 1H), 4.5-4.1 (m, 2H), 4.0-3.4 (m, 2H), 3.1-2.6 (m, 3H), 2.5-2.1 (m, 4H), 2.1-1.6 (m, 6H), 1.5-1.0 (m, 3H).

### Example 9

### (3S)-3-[5-[4-[(R)-(2-chlorophenyl)-phenyl-methyl]piperidine-1-carbonyl]-1-oxo-isoindolin-2-yl]piperidine-2,6-dione

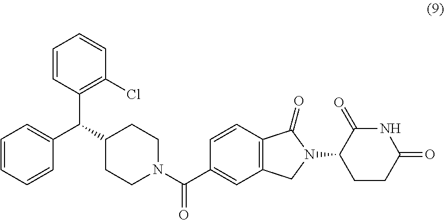 [SMILES: O=C1CC[C@H](N2Cc3cc(C(=O)N4CCC([C@H](c5ccccc5)c5ccccc5Cl)CC4)ccc3C2=O)C(=O)N1]

### Intermediate 9A: tert-butyl(4S)-5-amino-4-[5-[4-[(2-chlorophenyl)-phenyl-methyl]piperidine-1-carbonyl]-1-oxo-isoindolin-2-yl]-5-oxo-pentanoate

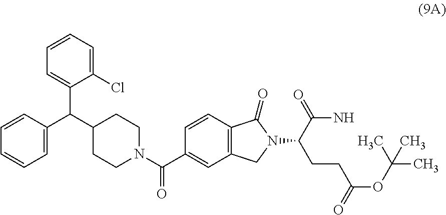 [SMILES: CC(C)(C)OC(=O)CC[C@@H](C(N)=O)N1Cc2cc(C(=O)N3CCC(C(c4ccccc4)c4ccccc4Cl)CC3)ccc2C1=O]

In a sealed vial under argon, a solution of 4-[(2-chlorophenyl)-phenyl-methyl]piperidine (515 mg, 0.883 mmol), 2-[(1S)-4-tert-butoxy-1-carbamoyl-4-oxo-butyl]-1-oxo-isoindoline-5-carboxylic acid (388 mg, 1.06 mmol) and DIPEA (617 μL, 3.53 mmol) in dry EtOAc (8.5 mL) was stirred at ambient temperature. A solution of T3P in EtOAc (50% w/w, 788 μL, 1.32 mmol) was added and the mixture was stirred at ambient temperature for 16 h. A saturated aqueous solution of NaHCO₃ was added and the aqueous layer was extracted with EtOAc (2×). The combined organic layers were washed with brine, dried over Na₂SO₄, and concentrated. The crude was purified by flash chromatography on silica gel using a gradient of EtOAc/EtOH (3/1) in n-heptane from 1% to 100%. Fractions were combined and concentrated under reduced pressure to afford tert-butyl(4S)-5-amino-4-[5-[4-[(2-chlorophenyl)-phenyl-methyl]piperidine-1-carbonyl]-1-oxo-isoindolin-2-yl]-5-oxo-pentanoate as a white foam (229 mg, 93% p., 38% y)). m/z [M+H]⁺=630.5, ¹H NMR (400 MHz, DMSO-d₆): δ (ppm) 7.71 (d, J=7.8 Hz, 2H), 7.58 (d, J=17.0 Hz, 2H), 7.45 (d, J=7.8 Hz, 1H), 7.42-7.12 (m, 9H), 4.76-4.71 (m, 1H), 4.61 (d, J=17.9 Hz, 1H), 4.47 (d, J=17.6 Hz, 2H), 4.17 (d, J=11.1 Hz, 1H), 3.51 (s, 1H), 3.01 (s, 1H), 2.77 (s, 1H), 2.68-2.56 (m, 1H), 2.21-2.10 (m, 3H), 2.05-1.93 (m, 1H), 1.64-1.36 (m, 2H), 1.31 (s, 10H), 1.07 (d, J=7.0 Hz, 1H).

### Intermediate 9B: tert-butyl(4S)-5-amino-4-[5-[4-[(R)-(2-chlorophenyl)-phenyl-methyl]piperidine-1-carbonyl]-1-oxo-isoindolin-2-yl]-5-oxo-pentanoate

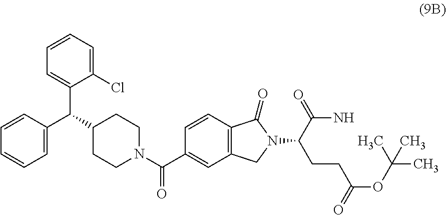 [SMILES: CC(C)(C)OC(=O)CC[C@@H](C(N)=O)N1Cc2cc(C(=O)N3CCC([C@H](c4ccccc4)c4ccccc4Cl)CC3)ccc2C1=O]

Tert-butyl(4S)-5-amino-4-[5-[4-[(2-chlorophenyl)-phenyl-methyl]piperidine-1-carbonyl]-1-oxo-isoindolin-2-yl]-5-oxo-pentanoate (223 mg) was purified by chiral supercritical chromatography (Waters Prep SFC200, Chiralpak AD (5 μm, 250×4.6 mm), CO₂/iPrOH 70/30 as mobile phase, flow rate 2.4 mL/min, 104 bars) to afford (first isomer eluted) tert-butyl(4S)-5-amino-4-[5-[4-[(R)-(2-chlorophenyl)-phenyl-methyl]piperidine-1-carbonyl]-1-oxo-isoindolin-2-yl]-5-oxo-pentanoate as a white solid (110 mg, 85% p., 44% y., d.e.>99.9%). m/z [M+H]⁺=630.4, ¹H NMR (400 MHz, DMSO-d₆): δ (ppm) 7.76-7.69 (m, 2H), 7.45 (d, J=7.7 Hz, 1H), 7.42-7.13 (m, 9H), 4.73 (dd, J=10.4, 4.0 Hz, 1H), 4.61 (d, J=17.9 Hz, 1H), 4.47 (d, J=17.6 Hz, 2H), 4.17 (d, J=11.2 Hz, 1H), 3.51 (s, 1H), 3.01 (s, 1H), 2.61 (d, J=11.2 Hz, 1H), 2.15 (d, J=8.7 Hz, 3H), 1.99 (s, 3H), 1.46 (s, 2H), 1.31 (s, 9H), 1.27-1.20 (m, 2H), 1.09 (s, 1H).

### Example 9

In a sealed vial, a solution of tert-butyl(4S)-5-amino-4-[5-[4-[(R)-(2-chlorophenyl)-phenyl-methyl]piperidine-1-carbonyl]-1-oxo-isoindolin-2-yl]-5-oxo-pentanoate (104 mg, 0.144 mmol) and 4-methylbenzenesulfonic acid hydrate (81.9 mg, 0.431 mmol) in acetonitrile (1.0 mL) was stirred at 85° C. for 16 h. A saturated aqueous solution of NaHCO₃ was added and the aqueous layer was extracted with EtOAc (2×). The combined organic layers were washed with brine, dried through hydrophobic paper, and concentrated under reduced pressure. The crude was purified by flash chromatography on silica gel using a gradient of EtOAc/EtOH (3/1) in cyclohexane from 10% to 100%. The desired fractions were concentrated under reduced pressure. The resulting solid was solubilized in the minimum amount of acetone and the homogenous solution was added dropwise to a stirred solution of n-pentane. The mixture was stirred for 2 h. The resulting precipitate was filtered, washed with n-pentane and dried under reduced pressure at 50° C. for 16 h to afford (3S)-3-[5-[4-[(R)-(2-chlorophenyl)-phenyl-methyl]piperidine-1-carbonyl]-1-oxo-isoindolin-2-yl]piperidine-2,6-dione as a white solid (34.0 mg, 97.6% p., 42% y.). m/z [M+H]⁺=556.3, ¹H NMR (500 MHz, DMSO-d₆): δ (ppm) 10.99 (s, 1H), 7.75 (d, J=7.8 Hz, 2H,), 7.61 (s, 1H), 7.49 (br d, J=7.5 Hz, 1H), 7.4-6.9 (m, 8H), 5.12 (dd, J=5.2, 13.3 Hz, 1H), 4.6-4.3 (m, 3H), 4.17 (br d, J=11.4 Hz, 1H,), 3.6-3.4 (m, 1H), 3.1-3.0 (m, 1H), 3.0-2.9 (m, 1H), 2.8-2.7 (m, 1H), 2.7-2.6 (m, 2H), 2.5-2.3 (m, 1H), 2.1-1.9 (m, 1H), 1.7-0.8 (m, 4H), [α]_{D}=−28° mL·dm⁻¹ g⁻¹[20° C., Na lamp, 589 nm, C=1.0 mg/mL, DMSO].

### Example 10

### (3S)-3-[5-(4-benzhydrylpiperidine-1-carbonyl)-6-(difluoromethyl)-1-oxo-isoindolin-2-yl]piperidine-2,6-dione

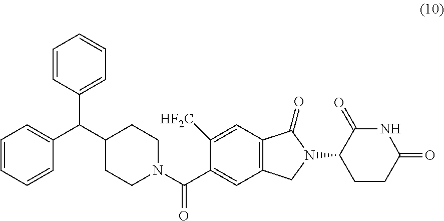

### Intermediate 10A: tert-butyl(4S)-5-amino-4-[5-(4-benzhydrylpiperidine-1-carbonyl)-1-oxo-6-vinyl-isoindolin-2-yl]-5-oxo-pentanoate

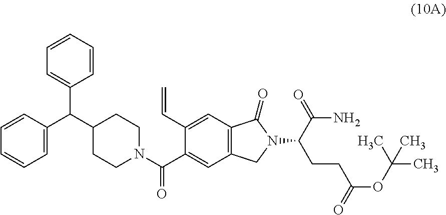 [SMILES: C=Cc1cc2c(cc1C(=O)N1CCC(C(c3ccccc3)c3ccccc3)CC1)CN([C@@H](CCC(=O)OC(C)(C)C)C(N)=O)C2=O]

To a solution of 4-benzhydrylpiperidine (605.0 mg, 2.41 mmol), 2-[(1S)-4-tert-butoxy-1-carbamoyl-4-oxo-butyl]-1-oxo-6-vinyl-isoindoline-5-carboxylic acid (1.00 g, 2.29 mmol), and DIPEA (1.6 mL, 9.17 mmol) in dry EtOAc (22 mL) was stirred at ambient temperature. A solution of T3P in EtOAc (50% w/w, 2.1 mL, 3.44 mmol) was added and the mixture was stirred at ambient temperature for 23 h. A saturated aqueous solution of NaHCO₃ was added and the aqueous layer was extracted with EtOAc (3×). The combined organic layers were washed with brine, dried over Na₂SO₄, and concentrated under reduced pressure. The crude was purified by flash chromatography on silica gel using a gradient of EtOAc/EtOH (3/1) in n-heptane from 1% to 100%. The fractions were combined and concentrated under reduced pressure to afford tert-butyl (4S)-5-amino-4-[5-(4-benzhydrylpiperidine-1-carbonyl)-1-oxo-6-vinyl-isoindolin-2-yl]-5-oxo-pentanoate as a yellow foam (417 mg, 91% p., 27% y.). m/z [M+H]⁺=622.5, ¹H NMR (400 MHz, DMSO-d₆): δ (ppm) 7.94 (d, J=8.6 Hz, 1H), 7.56 (s, 1H), 7.47-7.32 (m, 5H), 7.29 (t, J=7.6 Hz, 2H), 7.23 (t, J=7.6 Hz, 2H), 7.20-7.14 (m, 2H), 7.10 (t, J=7.3 Hz, 1H), 6.67 (ddd, J=42.5, 17.4, 11.1 Hz, 1H), 5.98 (dd, J=21.8, 17.5 Hz, 1H), 5.41 (dd, J=29.1, 11.2 Hz, 1H), 4.74 (d, J=10.5 Hz, 1H), 4.65-4.40 (m, 3H), 3.61 (d, J=11.1 Hz, 1H), 3.19 (d, J=13.2 Hz, 1H), 2.94 (q, J=12.6 Hz, 1H), 2.79 (t, J=12.5 Hz, 1H), 2.51 (s, 2H), 2.20-2.09 (m, 3H), 2.05-1.92 (m, 1H), 1.55 (s, 1H), 1.35-1.27 (m, 9H), 1.14-0.92 (m, 2H).

### Intermediate 10B: tert-butyl(4S)-5-amino-4-[5-(4-benzhydrylpiperidine-1-carbonyl)-6-formyl-1-oxo-isoindolin-2-yl]-5-oxo-pentanoate

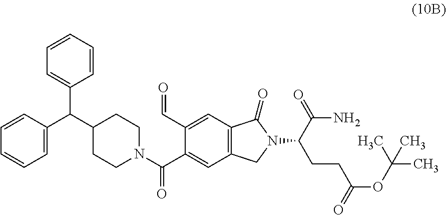 [SMILES: CC(C)(C)OC(=O)CC[C@@H](C(N)=O)N1Cc2cc(C(=O)N3CCC(C(c4ccccc4)c4ccccc4)CC3)c(C=O)cc2C1=O]

In a sealed vial, to a stirred solution of tert-butyl(4S)-5-amino-4-[5-(4-benzhydrylpiperidine-1-carbonyl)-1-oxo-6-vinyl-isoindolin-2-yl]-5-oxo-pentanoate (417.0 mg, 0.610 mmol) in a mixture of THF (16 mL) and water (3.6 mL) were added successively 2,6-lutidine (141 μL, 1.22 mmol), NaIO4 (522 mg, 2.44 mmol), and a 15 M aqueous solution of 0.0 (203 μL, 0.0305 mmol) at 0° C. The mixture was allowed to warm up to ambient temperature and stirred for 16 h. The reaction was quenched with the addition of a saturated aqueous solution of Na₂S203. EtOAc was added to the mixture. The layers were separated, and the aqueous layer was extracted with EtOAc. The combined organic layers were washed with brine, dried over Na₂SO₄, filtered, and concentrated under vacuum. The resulting gum was solubilized in the minimum amount of DCM and the homogenous solution was added dropwise to a stirred solution of n-pentane. The mixture was stirred overnight. The resulting precipitate was filtered, washed with n-pentane, and dried under vacuum to afford tert-butyl(4S)-5-amino-4-[5-(4-benzhydrylpiperidine-1-carbonyl)-6-formyl-1-oxo-isoindolin-2-yl]-5-oxo-pentanoate as a beige solid (393 mg, 90% p., 93% y.). m/z [M+H]⁺=624.5, ¹H NMR (400 MHz, DMSO-d₆): δ (ppm) 10.04 (s, 1H), 8.23 (s, 1H), 7.63 (s, 1H), 7.60 (s, 1H), 7.43-7.39 (m, 2H), 7.37-7.33 (m, 2H), 7.29 (t, J=7.6, 7.6 Hz, 2H), 7.22 (t, J=7.6, 7.6 Hz, 3H), 7.19-7.14 (m, 1H), 7.10 (t, J=7.4, 7.4 Hz, 1H), 4.78-4.66 (m, 2H), 4.58 (dd, J=18.8, 4.0 Hz, 1H), 4.50 (d, J=13.0 Hz, 1H), 3.60 (d, J=11.2 Hz, 1H), 3.21 (d, J=13.6 Hz, 1H), 2.94 (t, J=12.7, 12.7 Hz, 1H), 2.81 (t, J=12.3, 12.3 Hz, 1H), 2.54 (d, J=11.0 Hz, 1H), 2.17 (dq, J=10.7, 6.0, 6.0, 5.9 Hz, 3H), 1.99 (t, J=9.3, 9.3 Hz, 1H), 1.56 (d, J=13.0 Hz, 1H), 1.31 (d, J=5.3 Hz, 9H), 1.24 (s, 1H), 1.17 (d, J=12.7 Hz, 1H), 1.08-0.92 (m, 1H).

### Intermediate 10C: tert-butyl(4S)-5-amino-4-[5-(4-benzhydrylpiperidine-1-carbonyl)-6-(difluoromethyl)-1-oxo-isoindolin-2-yl]-5-oxo-pentanoate

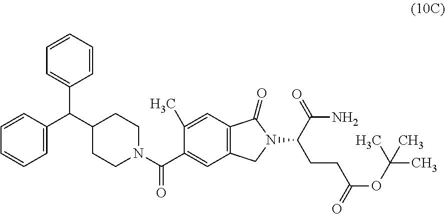 [SMILES: Cc1cc2c(cc1C(=O)N1CCC(C(c3ccccc3)c3ccccc3)CC1)CN([C@@H](CCC(=O)OC(C)(C)C)C(N)=O)C2=O]

In a sealed vial, to a stirred solution of tert-butyl(4S)-5-amino-4-[5-(4-benzhydrylpiperidine-1-carbonyl)-6-formyl-1-oxo-isoindolin-2-yl]-5-oxo-pentanoate (42.0 mg, 0.0606 mmol) in dry DCM (610 μL) at ambient temperature under argon was added DAST (101 μL, 0.727 mmol). The reaction mixture was stirred at ambient temperature for 16 h. The reaction was quenched with the addition of a saturated solution of NaHCO₃. The mixture was extracted with DCM (2×). The combined organic layers were washed brine, dried over Na₂SO₄, filtered, and concentrated under reduced pressure. The crude material was purified by flash chromatography on silica gel using a gradient of EtOAC/EtOH (3/1) in n-heptane from 1% to 100%. Fractions were combined and concentrated under reduced pressure to afford tert-butyl(4S)-5-amino-4-[5-(4-benzhydrylpiperidine-1-carbonyl)-6-(difluoromethyl)-1-oxo-isoindolin-2-yl]-5-oxo-pentanoate as an off-white solid (27.0 mg, 86% p., 59% y.). m/z [M+H]⁺=646.3.

### Example 10

In a sealed vial, a solution of tert-butyl(4S)-5-amino-4-[5-(4-benzhydrylpiperidine-1-carbonyl)-6-(difluoromethyl)-1-oxo-isoindolin-2-yl]-5-oxo-pentanoate (27.0 mg, 0.0417 mmol) and 4-methylbenzenesulfonic acid hydrate (23.9 mg, 0.125 mmol) in acetonitrile (270 μL) was stirred at 70° C. for 16 h. The mixture was cooled to ambient temperature and poured into a saturated aqueous solution of NaHCO₃. The aqueous layer was extracted with EtOAc (2×). The combined organic layers were washed with brine, filtered through hydrophobic paper, and concentrated. The crude was purified by flash chromatography on silica gel using a gradient of EtOAc/EtOH (3/1) in n-heptane from 0% to 100%. The desired fractions were concentrated under reduced pressure. The resulting gum was solubilized in the minimum amount of DCM and the homogenous solution was added to a stirred solution of n-pentane dropwise. The mixture was stirred for 2 h. The resulting precipitate was filtered, washed with n-pentane, and dried under reduced pressure at 50° C. for 16 h to afford (3S)-3-[5-(4-benzhydrylpiperidine-1-carbonyl)-6-(difluoromethyl)-1-oxo-isoindolin-2-yl]piperidine-2,6-dione as a white solid (5.5 mg, 90.9% p., 20% y.). m/z [M+H]⁺=572.5, ¹H NMR (600 MHz, DMSO-d₆): δ (ppm) 11.00 (s, 1H), 7.91 (s, 1H), 7.69-7.62 (m, 1H), 7.42-7.32 (m, 4H), 7.31-7.20 (m, 4H), 7.18-6.88 (m, 3H), 5.13 (dt, J=13.20, 4.11 Hz, 1H), 4.79-4.14 (m, 3H), 3.60 (br d, J=9.10 Hz, 1H), 3.28-2.71 (m, 4H), 2.64-2.52 (m, 2H), 2.44-2.34 (m, 1H), 2.07-1.95 (m, 1H), 1.62-0.86 (m, 4H).

### Example 11

### (3S)-3-[3-[4-[(2-chlorophenyl)-phenyl-methyl]piperidine-1-carbonyl]-7-oxo-5H-pyrrolo[3,4-b]pyridin-6-yl]piperidine-2,6-dione

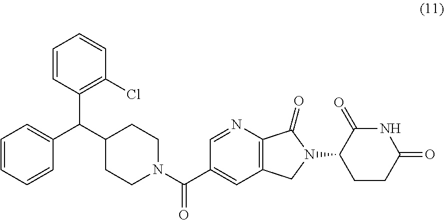 [SMILES: O=C1CC[C@H](N2Cc3cc(C(=O)N4CCC(C(c5ccccc5)c5ccccc5Cl)CC4)cnc3C2=O)C(=O)N1]

### Intermediate 11A: tert-butyl(4S)-5-amino-4-[3-[4-[(2-chlorophenyl)-phenyl-methyl]piperidine-1-carbonyl]-7-oxo-5H-pyrrolo[3,4-b]pyridin-6-yl]-5-oxo-pentanoate

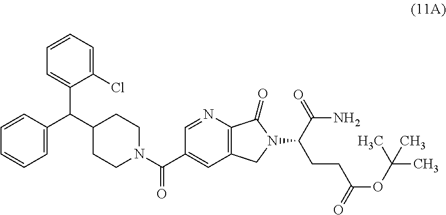 [SMILES: CC(C)(C)OC(=O)CC[C@@H](C(N)=O)N1Cc2cc(C(=O)N3CCC(C(c4ccccc4)c4ccccc4Cl)CC3)cnc2C1=O]

To a solution of 4-[(2-chlorophenyl)-phenyl-methyl]piperidine hydroiodide (132 mg, 0.316 mmol) in DMF (3.2 mL), were added 6-[(1S)-4-tert-butoxy-1-carbamoyl-4-oxo-butyl]-7-oxo-5H-pyrrolo[3,4-b]pyridine-3-carboxylic acid (115 mg, 0.316 mmol), a 0.5 M solution of HATU in DMF (948 μL, 0.474 mmol), and DIPEA (276 μL, 1.58 mmol). The reaction mixture was stirred at ambient temperature for 16 h. Water was added and the aqueous layer was extracted with EtOAc (2×). The organic layer was washed with water, brine, dried over Na₂SO₄, filtered, and concentrated under reduced pressure. The crude was purified by flash chromatography on silica gel using a gradient of EtOAc/EtOH (3/1) in n-heptane from 5% to 50%. The desired fractions were combined and concentrated under reduced pressure to afford tert-butyl(4S)-5-amino-4-[3-[4-[(2-chlorophenyl)-phenyl-methyl]piperidine-1-carbonyl]-7-oxo-5H-pyrrolo[3,4-b]pyridin-6-yl]-5-oxo-pentanoate as an off-white powder (131 mg, 97% p., 64% y.). m/z [M+H]⁺=631.6, ¹H NMR (400 MHz, DMSO-d₆): δ (ppm) 8.70 (d, J=1.9 Hz, 1H), 8.08 (s, 1H), 7.74 (s, 1H), 7.60 (s, 1H), 7.47-7.08 (m, 9H), 4.77 (dd, J=10.5, 4.0 Hz, 1H), 4.61 (d, J=18.5 Hz, 1H), 4.49 (d, J=18.5 Hz, 2H), 4.18 (d, J=11.2 Hz, 1H), 3.50 (s, 1H), 3.07 (d, J=14.3 Hz, 1H), 2.79 (d, J=12.7 Hz, 1H), 2.71-2.56 (m, 1H), 2.24-2.12 (m, 3H), 2.04-1.92 (m, 1H), 1.53 (d, J=44.6 Hz, 1H), 1.31 (s, 9H).

### Example 11

In a sealed vial under argon, a solution of tert-butyl(4S)-5-amino-4-[3-[4-[(2-chlorophenyl)-phenyl-methyl]piperidine-1-carbonyl]-7-oxo-5H-pyrrolo[3,4-b]pyridin-6-yl]-5-oxo-pentanoate (131 mg, 0.201 mmol) in dry acetonitrile (1.2 mL) was stirred at ambient temperature. 4-Methylbenzenesulfonic acid hydrate (191 mg, 1.01 mmol) was added and the solution was stirred at 75° C. for 3 h. The mixture was cooled to ambient temperature, diluted with EtOAc, and a saturated aqueous solution NaHCO₃ was added dropwise. Water was added and the mixture was extracted with EtOAc (2×). The combined organic layers were washed with brine, dried over Na₂SO₄, filtered, and concentrated. The crude was purified by reverse-phase preparative chromatography using a gradient of acetonitrile in water from 0% to 100% (0.1% AcOH). Desired fractions were combined, and acetonitrile was removed under reduced pressure. The suspension was filtered, washed with water and dried under reduced pressure at 50° C. for 16 h to afford (3S)-3-[3-[4-[(2-chlorophenyl)-phenyl-methyl]piperidine-1-carbonyl]-7-oxo-5H-pyrrolo[3,4-b]pyridin-6-yl]piperidine-2,6-dione as a white solid (22.7 mg, 98.7% p., 20% y.). m/z [M+H]⁺=557.3, ¹H NMR (600 MHz, DMSO-d₆): δ (ppm) 11.01 (s, 1H), 8.73 (s, 1H), 8.10 (br s, 1H), 7.84-7.66 (m, 1H), 7.46-7.07 (m, 8H), 5.16 (dd, J=13.4, 5.1 Hz, 1H), 4.60-4.30 (m, 3H), 4.25-4.09 (m, 1H), 3.51 (ddd, J=4.4, 2.4, 1.2 Hz, 1H), 3.18-2.98 (m, 1H), 2.90 (ddd, J=17.5, 13.6, 5.4 Hz, 1H), 2.71-2.85 (m, 1H), 2.68-2.57 (m, 2H), 2.47-2.34 (m, 1H), 2.06-1.96 (m, 1H), 1.66-1.08 (m, 4H).

### Example 12

### (3S)-3-[5-[4-[(R)-(2-chlorophenyl)-phenyl-methyl]piperidine-1-carbonyl]-6-methyl-1-oxo-isoindolin-2-yl]piperidine-2,6-dione

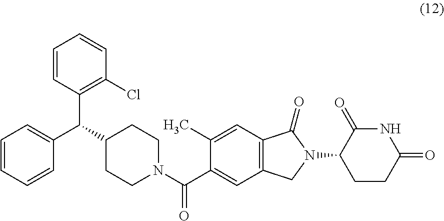 [SMILES: Cc1cc2c(cc1C(=O)N1CCC([C@H](c3ccccc3)c3ccccc3Cl)CC1)CN([C@H]1CCC(=O)NC1=O)C2=O]

### Intermediate 12A: tert-butyl(4S)-5-amino-4-[5-[4-[(R)-(2-chlorophenyl)-phenyl-methyl]piperidine-1-carbonyl]-6-methyl-1-oxo-isoindolin-2-yl]-5-oxo-pentanoate

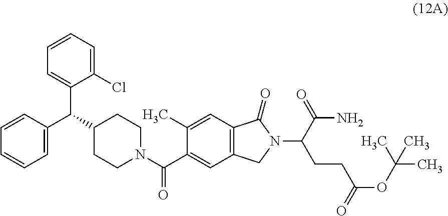 [SMILES: Cc1cc2c(cc1C(=O)N1CCC([C@H](c3ccccc3)c3ccccc3Cl)CC1)CN(C(CCC(=O)OC(C)(C)C)C(N)=O)C2=O]

To a stirred suspension of 2-[(1S)-4-tert-butoxy-1-carbamoyl-4-oxo-butyl]-6-methyl-1-oxo-isoindoline-5-carboxylic acid (240 mg, 0.638 mmol) in EtOAc (6.0 mL) were added successively 4-[(R)-(2-chlorophenyl)-phenyl-methyl]piperidine hydroiodide (290 mg, 0.701 mmol), DIPEA (557 μL, 3.19 mmol), and a solution of T3P in EtOAc (50% w/w, 759 μL, 1.28 mmol). The reaction mixture was stirred at ambient temperature overnight. The reaction was quenched with the addition of a saturated aqueous solution of NaHCO₃. The aqueous layer was extracted with EtOAc (2×). The combined organic layers were washed with brine, dried over Na₂SO₄, filtered, and concentrated under reduced pressure to afford tert-butyl(4S)-5-amino-4-[5-[4-[(R)-(2-chlorophenyl)-phenyl-methyl]piperidine-1-carbonyl]-6-methyl-1-oxo-isoindolin-2-yl]-5-oxo-pentanoate as a light orange solid (406 mg, 92% p. 91% y.). m/z [M+H]⁺=644.3, ¹H NMR (400 MHz, DMSO-d₆): δ (ppm) 7.71 (dd, J=21.4, 7.9 Hz, 1H), 7.56 (d, J=8.9 Hz, 2H), 7.51-7.09 (m, 10H), 4.72 (dd, J=10.3, 3.9 Hz, 1H), 4.63-4.48 (m, 2H), 4.42 (d, J=17.5 Hz, 1H), 4.15 (dd, J=11.0, 7.4 Hz, 1H), 3.22 (t, J=15.4 Hz, 1H), 2.97 (q, J=13.5 Hz, 1H), 2.77 (t, J=11.4 Hz, 1H), 2.59 (d, J=11.5 Hz, 1H), 2.38-2.20 (m, 3H), 2.15 (t, J=3.8 Hz, 3H), 1.97 (s, 1H), 1.68-1.45 (m, 1H), 1.45-1.26 (m, 10H), 1.12 (d, J=7.5 Hz, 2H), [α]_{D}=−11°·mL·dm⁻¹.g⁻¹[20° C., Na lamp, 589 nm, C=1.3 mg/mL, DMSO].

### Example 12

To a stirred solution of tert-butyl(4S)-5-amino-4-[5-[4-[(R)-(2-chlorophenyl)-phenyl-methyl]piperidine-1-carbonyl]-6-methyl-1-oxo-isoindolin-2-yl]-5-oxo-pentanoate (406 mg, 0.580 mmol) in acetonitrile (3.0 mL) at ambient temperature was added 4-methylbenzenesulfonic acid hydrate (551 mg, 2.90 mmol). The reaction mixture was stirred at 80° C. for 1 h. The reaction was quenched with the addition of a saturated aqueous solution of NaHCO₃. EtOAc was added to the mixture. The mixture was extracted with EtOAc (2×). The combined organic layers were washed with brine, dried over Na₂SO₄, filtered, and concentrated under reduced pressure. The crude was purified by flash chromatography on silica gel using a gradient of EtOAc/EtOH (3/1) in cyclohexane from 10% to 100%. The resulting gum was solubilized in a minimum of acetone and added dropwise to a stirred solution of n-pentane. The resulting precipitate was filtered, washed with n-pentane, and dried under reduced pressure at 50° C. for 12 h to afford (3S)-3-[5-[4-[(R)-(2-chlorophenyl)-phenyl-methyl]piperidine-1-carbonyl]-6-methyl-1-oxo-isoindolin-2-yl]piperidine-2,6-dione as a white powder (140 mg, 99.7% p.,42% y.). m/z [M+H]⁺=570.3, ¹H NMR (500 MHz, DMSO-d₆): δ (ppm) 10.98 (s, 1H), 7.80-7.66 (m, 1H), 7.61 (br d, J=5.8 Hz, 1H), 7.52-7.11 (m, 9H), 5.15-5.05 (m, 1H), 4.60-4.48 (m, 1H), 4.48-4.24 (m, 2H), 4.19-4.12 (m, 1H), 3.31-3.17 (m, 1H), 3.04-2.86 (m, 2H), 2.83-2.72 (m, 1H), 2.66-2.55 (m, 2H), 2.43-2.30 (m, 2H), 2.25 (s, 2H), 2.07-1.91 (m, 1H), 1.71-0.90 (m, 4H), [α]_{D}=−25°·mL·dm⁻¹ g⁻¹[20° C., Na lamp, 589 nm, C=1.2 mg/mL, DMSO].

### Example 13

### (3S)-3-[3-[4-[(R)-(2-chloro-3-methoxy-phenyl)-phenyl-methyl]piperidine-1-carbonyl]-7-oxo-5H-pyrrolo[3,4-b]pyridin-6-yl]piperidine-2,6-dione

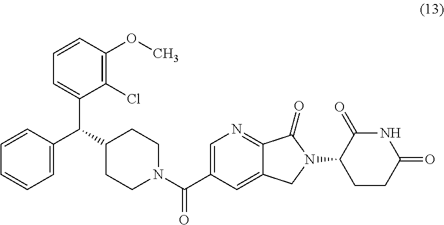 [SMILES: COc1cccc([C@@H](c2ccccc2)C2CCN(C(=O)c3cnc4c(c3)CN([C@H]3CCC(=O)NC3=O)C4=O)CC2)c1Cl]

### Intermediate 13A: tert-butyl(4S)-5-amino-4-[3-[4-[(R)-(2-chloro-3-methoxy-phenyl)-phenyl-methyl]piperidine-1-carbonyl]-7-oxo-5H-pyrrolo[3,4-b]pyridin-6-yl]-5-oxo-pentanoate

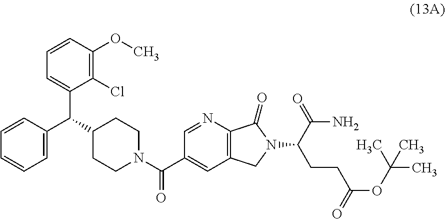 [SMILES: COc1cccc([C@@H](c2ccccc2)C2CCN(C(=O)c3cnc4c(c3)CN([C@@H](CCC(=O)OC(C)(C)C)C(N)=O)C4=O)CC2)c1Cl]

To a stirred suspension of 6-[(1S)-4-tert-butoxy-1-carbamoyl-4-oxo-butyl]-7-oxo-5H-pyrrolo[3,4-b]pyridine-3-carboxylic acid (75.0 mg, 0.206 mmol) in EtOAc (2.1 mL) were added successively 4-[(R)-(2-chloro-3-methoxy-phenyl)-phenyl-methyl]piperidine hydroiodide (101 mg, 0.227 mmol), DIPEA (180 μL, 1.03 mmol), and then a solution of T3P in MeTHF (50% w/w, 197 mg, 0.310 mmol). The reaction mixture was stirred at ambient temperature overnight. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by flash chromatography on silica gel using a gradient of EtOAc/EtOH (3/1) in cyclohexane from 2% to 100% to afford tert-butyl(4S)-5-amino-4-[3-[4-[(R)-(2-chloro-3-methoxy-phenyl)-phenyl-methyl]piperidine-1-carbonyl]-7-oxo-5H-pyrrolo[3,4-b]pyridin-6-yl]-5-oxo-pentanoate as an off-white solid (89.0 mg, 100% p., 65% y.). m/z [M+H]⁺=661.3, ¹H NMR (400 MHz, DMSO-d₆): δ (ppm) 8.70 (s, 1H), 8.07 (s, 1H), 7.60 (s, 1H), 7.27 (qd, J=26.3, 10.6 Hz, 8H), 6.95 (s, 1H), 4.77 (dd, J=10.4, 3.9 Hz, 1H), 4.61 (d, J=19.4 Hz, 1H), 4.49 (d, J=18.2 Hz, 2H), 4.23 (d, J=11.2 Hz, 1H), 3.80 (d, J=11.0 Hz, 3H), 3.49 (s, 1H), 3.07 (d, J=13.1 Hz, 1H), 2.79 (d, J=12.0 Hz, 1H), 2.59 (d, J=11.3 Hz, 1H), 2.17 (d, J=7.8 Hz, 3H), 1.99-1.95 (m, 1H), 1.70-0.97 (m, 13H).

### Example 13

To a stirred solution of tert-butyl(4S)-5-amino-4-[3-[4-[(R)-(2-chloro-3-methoxy-phenyl)-phenyl-methyl]piperidine-1-carbonyl]-7-oxo-5H-pyrrolo[3,4-b]pyridin-6-yl]-5-oxo-pentanoate (89.0 mg, 0.135 mmol) in acetonitrile (1.3 mL) was added 4-methylbenzenesulfonic acid hydrate (128 mg, 0.673 mmol). The reaction mixture was stirred at 80° C. for 4 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by flash chromatography on silica gel using a gradient of a EtOAc/EtOH (3/1) in cyclohexane from 10% to 100%. The residue was solubilized in the minimum amount of DCM and the homogenous solution was added to n-pentane dropwise. The resulting precipitate was triturated for 1 h, filtered, washed with n-pentane, and dried under reduced pressure at 50° C. overnight to afford (3S)-3-[3-[4-[(R)-(2-chloro-3-methoxy-phenyl)-phenyl-methyl]piperidine-1-carbonyl]-7-oxo-5H-pyrrolo[3,4-b]pyridin-6-yl]piperidine-2,6-dione as a white solid (39.6 mg, 100% p., 50% y.). m/z [M+H]⁺=587.4, ¹H NMR (500 MHz, DMSO-d₆): δ (ppm) 11.02 (s, 1H), 8.74 (s, 1H), 8.10 (br s, 1H), 7.37 (br d, J=8.63 Hz, 2H), 7.34-7.23 (m, 4H), 7.23-7.09 (m, 1H), 6.97 (br d, J=1.38 Hz, 1H), 5.17 (dd, J=13.26, 5.13 Hz, 1H), 4.59-4.33 (m, 3H), 4.23 (br d, J=10.76 Hz, 1H), 3.80 (br d, J=14.13 Hz, 3H), 3.63-3.48 (m, 1H), 3.17-2.99 (m, 1H), 2.91 (ddd, J=17.54, 13.60, 5.50 Hz, 1H),2.86-2.72 (m, 1H), 2.69-2.56 (m, 2H), 2.43 (dd, J=13.07, 4.69 Hz, 1H), 2.08-1.97 (m, 1H), 1.68-1.47 (m, 1H), 1.47-1.30 (m, 1H), 1.30-1.20 (m, 1H), 1.19-1.06 (m, 1H).

### Example 14

### (3S)-3-[3-[4-[(S)-(2-chloro-3-methoxy-phenyl)-phenyl-methyl]piperidine-1-carbonyl]-7-oxo-5H-pyrrolo[3,4-b]pyridin-6-yl]piperidine-2,6-dione

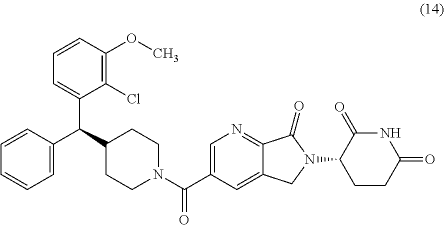 [SMILES: COc1cccc([C@H](c2ccccc2)C2CCN(C(=O)c3cnc4c(c3)CN([C@H]3CCC(=O)NC3=O)C4=O)CC2)c1Cl]

### Intermediate 14A: tert-butyl(4S)-5-amino-4-[3-[4-[(S)-(2-chloro-3-methoxy-phenyl)-phenyl-methyl]piperidine-1-carbonyl]-7-oxo-5H-pyrrolo[3,4-b]pyridin-6-yl]-5-oxo-pentanoate

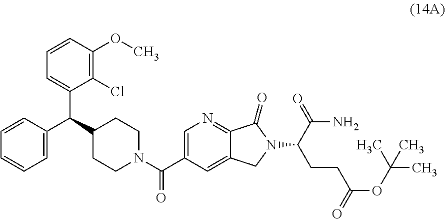 [SMILES: COc1cccc([C@H](c2ccccc2)C2CCN(C(=O)c3cnc4c(c3)CN([C@@H](CCC(=O)OC(C)(C)C)C(N)=O)C4=O)CC2)c1Cl]

To a stirred suspension of 6-[(1S)-4-tert-butoxy-1-carbamoyl-4-oxo-butyl]-7-oxo-5H-pyrrolo[3,4-b]pyridine-3-carboxylic acid (75.0 mg, 0.206 mmol) in EtOAc (2.1 mL) were added successively 4-[(S)-(2-chloro-3-methoxy-phenyl)-phenyl-methyl]piperidine hydroiodide (101 mg, 0.227 mmol), DIPEA (180 μL, 1.03 mmol), and then a solution of T3P in MeTHF (50% w/w, 197 mg, 0.310 mmol). The reaction mixture was stirred at ambient temperature overnight. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by flash chromatography on silica gel using a gradient of EtOAc/EtOH (3/1) in cyclohexane from 2% to 100% to afford tert-butyl(4S)-5-amino-4-[3-[4-[(S)-(2-chloro-3-methoxy-phenyl)-phenyl-methyl]piperidine-1-carbonyl]-7-oxo-5/I-pyrrolo[3,4-b]pyridin-6-yl]-5-oxo-pentanoate as an off-white solid (89.3 mg, 100% p., 65% y.). m/z [M+H]⁺=661.4, ¹H NMR (400 MHz, DMSO-d₆): δ (ppm) 8.70 (s, 1H), 8.07 (s, 1H), 7.60 (s, 1H), 7.27 (dt, J=38.6, 26.2 Hz, 8H), 6.96 (s, 1H), 4.83-4.70 (m, 1H), 4.61 (d, J=18.1 Hz, 1H), 4.50 (d, J=20.2 Hz, 2H), 4.23 (d, J=11.1 Hz, 1H), 3.80 (d, J=10.0 Hz, 3H), 3.50 (s, 1H), 3.07 (d, J=15.3 Hz, 1H), 2.80 (s, 1H), 2.59 (d, J=10.7 Hz, 1H), 2.19 (d, J=2.5 Hz, 3H), 2.07-1.87 (m, 1H), 1.72-1.00 (m, 13H).

### Example 14

To a stirred solution of tert-butyl(4S)-5-amino-4-[3-[4-[(S)-(2-chloro-3-methoxy-phenyl)-phenyl-methyl]piperidine-1-carbonyl]-7-oxo-5H-pyrrolo[3,4-b]pyridin-6-yl]-5-oxo-pentanoate (89.0 mg, 0.135 mmol) in acetonitrile (1.3 mL) was added 4-methylbenzenesulfonic acid hydrate (128 mg, 0.673 mmol). The reaction mixture was stirred at 80° C. for 4 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by flash chromatography on silica gel using a gradient of a EtOAc/EtOH (3/1) in cyclohexane from 10% to 100%. The residue was solubilized in the minimum amount of DCM and the homogenous solution was added to n-pentane dropwise. The resulting precipitate was triturated for 1 h, filtered, washed with n-pentane, and dried under reduced pressure at 50° C. overnight to afford (3S)-3-[3-[4-[(S)-(2-chloro-3-methoxy-phenyl)-phenyl-methyl]piperidine-1-carbonyl]-7-oxo-5H-pyrrolo[3,4-b]pyridin-6-yl]piperidine-2,6-dione as a white solid (33.4 mg, 100% p., 42% y.). m/z [M+H]⁺=587.4, ¹H NMR (500 MHz, DMSO-d₆): δ (ppm) 11.02 (s, 1H), 8.74 (s, 1H), 8.11 (br s, 1H), 7.42-7.35 (m, 2H), 7.35-7.23 (m, 4H), 7.22-7.09 (m, 1H), 7.04-6.88 (m, 1H), 5.17 (dd, J=13.3, 5.1 Hz, 1H), 4.59-4.34 (m, 3H), 4.31-4.12 (m, 1H), 3.89-3.73 (m, 3H), 3.62-3.45 (m, 1H), 3.19-2.99 (m, 1H), 2.91 (ddd, J=17.5, 13.6, 5.3 Hz, 1H), 2.86-2.72 (m, 1H), 2.68-2.55 (m, 2H), 2.49-2.36 (m, 1H), 2.08-1.97 (m, 1H), 1.68-1.05 (m, 4H).

### Example 15

### (3S)-3-[3-[4-[(R)-(2-chlorophenyl)-phenyl-methyl]piperidine-1-carbonyl]-7-oxo-5H-pyrrolo[3,4-b]pyridin-6-yl]piperidine-2,6-dione

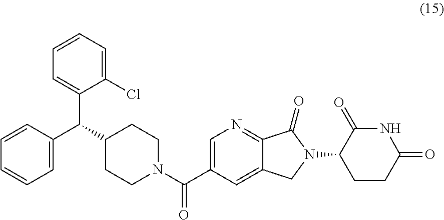 [SMILES: O=C1CC[C@H](N2Cc3cc(C(=O)N4CCC([C@H](c5ccccc5)c5ccccc5Cl)CC4)cnc3C2=O)C(=O)N1]

### Intermediate 15A: tert-butyl(4S)-5-amino-4-[3-[4-[(R)-(2-chlorophenyl)-phenyl-methyl]piperidine-1-carbonyl]-7-oxo-5H-pyrrolo[3,4-b]pyridin-6-yl]-5-oxo-pentanoate

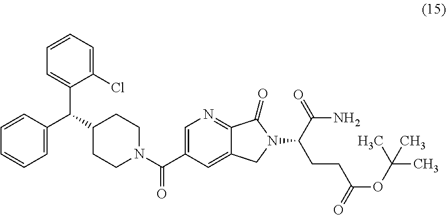 [SMILES: CC(C)(C)OC(=O)CC[C@@H](C(N)=O)N1Cc2cc(C(=O)N3CCC([C@H](c4ccccc4)c4ccccc4Cl)CC3)cnc2C1=O]

To a stirred solution of 6-[(1S)-4-tert-butoxy-1-carbamoyl-4-oxo-butyl]-7-oxo-5H-pyrrolo[3,4-b]pyridine-3-carboxylic acid (120 mg, 0.330 mmol) in EtOAc (1.6 mL) at ambient temperature were added a solution of T3P in EtOAc (50% w/w, 252 mg, 0.396 mmol), DIPEA (138 μL, 0.789 mmol), and 4-[(R)-(2-chlorophenyl)-phenyl-methyl]piperidine hydroiodide (167 mg, 0.363 mmol). The reaction mixture was stirred at ambient temperature for 16 h. The reaction was quenched with the addition of a saturated aqueous solution of NaHCO₃. EtOAc was added to the mixture. The aqueous layer was extracted with EtOAc (2×). The combined organic layers were washed with brine, dried over Na₂SO₄, filtered, and concentrated. The crude was purified by flash chromatography on silica gel using a gradient of EtOAc/EtOH (3/1) in cyclohexane from 10% to 100% to afford tert-butyl(4S)-5-amino-4-[3-[4-[(R)-(2-chlorophenyl)-phenyl-methyl]piperidine-1-carbonyl]-7-oxo-5H-pyrrolo[3,4-b]pyridin-6-yl]-5-oxo-pentanoate as a white solid (105 mg, 100% p.,50% y.). m/z [M+H]⁺=631.4, ¹H NMR (400 MHz, DMSO-d₆): δ (ppm) 8.70 (s, 1H), 8.08 (d, J=1.8 Hz, 1H), 7.74 (m, 1H), 7.60 (s, 1H), 7.27 (m, 9H), 4.77 (dd, J=10.5, 3.9 Hz, 1H), 4.61 (d, J=18.5 Hz, 1H), 4.49 (d, J=18.8 Hz, 2H), 4.17 (d, J=11.1 Hz, 1H), 3.49 (s, 1H), 3.07 (d, J=13.1 Hz, 1H), 2.77 (m, 1H), 2.64 (m, 1H), 2.17 (m, 3H), 1.98 (m, 1H), 1.53 (m, 1H), 1.31 (s, 9H), 1.17 (m, 3H).

### Example 15

To a stirred solution of tert-butyl(4S)-5-amino-4-[3-[4-[(R)-(2-chlorophenyl)-phenyl-methyl]piperidine-1-carbonyl]-7-oxo-5H-pyrrolo[3,4-b]pyridin-6-yl]-5-oxo-pentanoate (105 mg, 0.166 mmol) in acetonitrile (1.1 mL) at ambient temperature was added 4-methylbenzenesulfonic acid hydrate (63.3 mg, 0.333 mmol). The reaction mixture was stirred at 80° C. for 3 h. The reaction mixture was cooled to ambient temperature and diluted with a saturated aqueous solution of NaHCO₃. The aqueous layer was extracted with EtOAc (2×). The combined organic layers were washed with brine, dried over Na₂SO₄, filtered, and concentrated under reduced pressure. The crude was purified by flash chromatography on silica gel using a gradient of a EtOAc/EtOH (3/1) in cyclohexane from 10% to 100%. The residue was solubilized in a minimum amount of acetone and the homogenous solution was added to a stirred solution of n-pentane dropwise. The resulting precipitate was triturated for 1 h, filtered, washed with n-pentane, and dried under reduced pressure at 50° C. overnight to afford (3S)-3-[3-[4-[(R)-(2-chlorophenyl)-phenyl-methyl]piperidine-1-carbonyl]-7-oxo-5H-pyrrolo[3,4-b]pyridin-6-yl]piperidine-2,6-dione as a white powder (6.0 mg, 99.3% p., 6% y.). m/z [M+H]⁺=557.4, ¹H NMR (500 MHz, DMSO-d₆): δ (ppm) 11.02 (s, 1H), 8.74 (s, 1H), 8.11 (br s, 1H), 7.8-7.7 (m, 1H), 7.6-6.9 (m, 8H), 5.17 (dd, J=13.3, 5.1 Hz, 1H), 4.8-4.3 (m, 3H), 4.18 (br dd, J=9.6-1.5 Hz, 1H), 3.7-3.4 (m, 1H), 3.2-3.0 (m, 1H), 3.0-2.7 (m, 2H), 2.7-2.6 (m, 2H), 2.43 (br dd, J=13.1, 4.6 Hz, 1H), 2.1-1.9 (m, 1H), 1.8-0.9 (m, 4H).

### Example 16

### (3S)-3-[3-[4-[(S)-(2-chlorophenyl)-phenyl-methyl]piperidine-1-carbonyl]-7-oxo-5H-pyrrolo[3,4-b]pyridin-6-yl]piperidine-2,6-dione

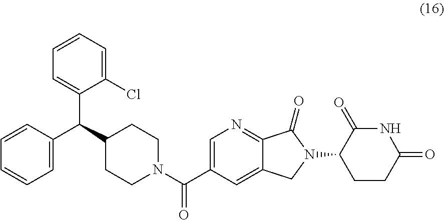 [SMILES: O=C1CC[C@H](N2Cc3cc(C(=O)N4CCC([C@@H](c5ccccc5)c5ccccc5Cl)CC4)cnc3C2=O)C(=O)N1]

### Intermediate 16A: tert-butyl(4S)-5-amino-4-[3-[4-[(S)-(2-chlorophenyl)-phenyl-methyl]piperidine-1-carbonyl]-7-oxo-5H-pyrrolo[3,4-b]pyridin-6-yl]-5-oxo-pentanoate

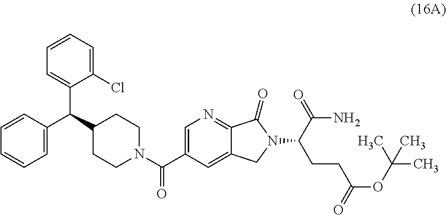 [SMILES: CC(C)(C)OC(=O)CC[C@@H](C(N)=O)N1Cc2cc(C(=O)N3CCC([C@@H](c4ccccc4)c4ccccc4Cl)CC3)cnc2C1=O]

To a stirred solution of 6-[(1S)-4-tert-butoxy-1-carbamoyl-4-oxo-butyl]-7-oxo-5H-pyrrolo[3,4-b]pyridine-3-carboxylic acid (120 mg, 0.330 mmol) in EtOAc (1.6 mL) at ambient temperature were added a solution of T3P in EtOAc (50% w/w, 252 mg, 0.396 mmol), DIPEA (138 μL, 0.789 mmol), and 4-[(S)-(2-chlorophenyl)-phenyl-methyl]piperidine hydroiodide (167 mg, 0.363 mmol). The reaction mixture was stirred at ambient temperature for 16 h. The reaction mixture was cooled to ambient temperature and diluted with a saturated aqueous solution of NaHCO₃. The aqueous layer was extracted with EtOAc (2×). The combined organic layers were washed with brine, dried over Na₂SO₄, filtered, and concentrated under reduced pressure. The crude was purified by flash chromatography on silica gel using a gradient of a EtOAc/EtOH (3/1) in cyclohexane from 10% to 100%. The residue was solubilized in a minimum amount of acetone and the homogenous solution was added to a stirred solution of n-pentane dropwise. The resulting precipitate was triturated for 1 h, filtered, washed with n-pentane, and dried under reduced pressure at 50° C. overnight to afford tert-butyl(4S)-5-amino-4-[3-[4-[(S)-(2-chlorophenyl)-phenyl-methyl]piperidine-1-carbonyl]-7-oxo-5H-pyrrolo[3,4-b]pyridin-6-yl]-5-oxo-pentanoate as a white solid (87.5 mg, 98% p., 41% y.). m/z [M+H]⁺=631.4, ¹H NMR (400 MHz, DMSO-d₆): δ (ppm) 8.70 (d, J=1.8 Hz, 1H), 8.08 (s, 1H), 7.75 (m, 1H), 7.60 (s, 1H), 7.29 (m, 9H), 4.78 (m, 1H), 4.61 (m, 1H), 4.49 (m, 2H), 4.17 (d, J=11.1 Hz, 1H), 3.50 (m, 1H), 3.07 (m, 1H), 2.79 (m, 1H), 2.64 (m, 1H), 2.18 (m, 3H), 1.99 (m, 1H), 1.59 (m, 1H), 1.39 (m, 1H), 1.31 (s, 9H), 1.18 (m, 2H).

### Example 16

To a stirred solution of tert-butyl(4S)-5-amino-4-[3-[4-[(S)-(2-chlorophenyl)-phenyl-methyl]piperidine-1-carbonyl]-7-oxo-5H-pyrrolo[3,4-b]pyridin-6-yl]-5-oxo-pentanoate (89.3 mg, 0.139 mmol) in acetonitrile (1.0 mL) at ambient temperature was added 4-methylbenzenesulfonic acid hydrate (52.7 mg, 0.277 mmol). The reaction mixture was stirred at 80° C. for 3 h. The reaction mixture was cooled to ambient temperature and diluted with a saturated aqueous solution of NaHCO₃. The aqueous layer was extracted with EtOAc (2×). The combined organic layers were washed with brine, dried over Na₂SO₄, filtered, and concentrated under reduced pressure. The crude was purified by flash chromatography on silica gel using a gradient of a EtOAc/EtOH (3/1) in cyclohexane from 10% to 100%. The residue was solubilized in a minimum amount of acetone and the homogenous solution was added to a stirred solution of n-pentane dropwise. The resulting precipitate was triturated for 1 h, filtered, washed with n-pentane, and dried under reduced pressure at 50° C. overnight to afford (3S)-3-[3-[4-[(S)-(2-chlorophenyl)-phenyl-methyl]piperidine-1-carbonyl]-7-oxo-5H-pyrrolo[3,4-b]pyridin-6-yl]piperidine-2,6-dione as a white powder (6.9 mg, 98.8% p., 9% y.). m/z [M+H]⁺=557.3, ¹H NMR (600 MHz, DMSO-d₆): δ (ppm) 11.01 (s, 1H), 8.73 (s, 1H), 8.10 (br s, 1H), 7.77-7.70 (m, 1H), 7.40-7.34 (m, 3H), 7.34-7.24 (m, 3H), 7.24-7.13 (m, 2H), 5.16 (dd, J=13.4, 5.1 Hz, 1H), 4.54-4.45 (m, 2H), 4.43-4.36 (m, 1H), 4.18 (br d, J=10.3 Hz, 1H), 3.51 (br s, 1H), 3.13-3.00 (m, 1H), 2.90 (ddd, J=17.4, 13.6, 5.5 Hz, 1H), 2.85-2.75 (m, 1H), 2.66-2.57 (m, 2H), 2.46-2.38 (m, 1H), 2.04-1.98 (m, 1H), 1.65-1.28 (m, 2H), 1.26-1.11 (m, 2H).

### Example 17

### (3R)-3-[5-[4-[(R)-(2-chlorophenyl)-phenyl-methyl]piperidine-1-carbonyl]-6-methyl-1-oxo-isoindolin-2-yl]piperidine-2,6-dione

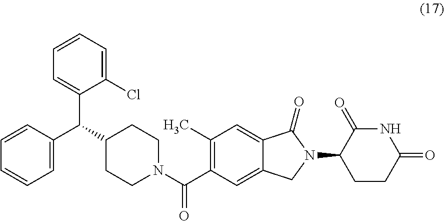 [SMILES: Cc1cc2c(cc1C(=O)N1CCC([C@H](c3ccccc3)c3ccccc3Cl)CC1)CN([C@@H]1CCC(=O)NC1=O)C2=O]

### Intermediate 17A: tert-butyl(4R)-5-amino-4-[5-[4-[(R)-(2-chlorophenyl)-phenyl-methyl]piperidine-1-carbonyl]-6-methyl-1-oxo-isoindolin-2-yl]-5-oxo-pentanoate

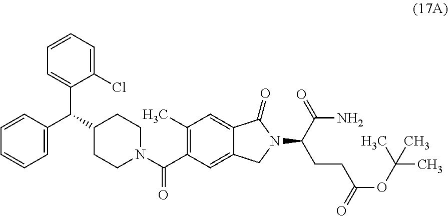 [SMILES: Cc1cc2c(cc1C(=O)N1CCC([C@H](c3ccccc3)c3ccccc3Cl)CC1)CN([C@H](CCC(=O)OC(C)(C)C)C(N)=O)C2=O]

In a vial under nitrogen were successively charged 2-[(1R)-4-tert-butoxy-1-carbamoyl-4-oxo-butyl]-6-methyl-1-oxo-isoindoline-5-carboxylic acid (393 mg, 1.04 mmol) and 4-[(R)-(2-chlorophenyl)-phenyl-methyl]piperidine hydroiodide (450 mg, 1.07 mmol) in dry DCM (4.3 mL). The mixture was cooled down to 0° C. DIPEA (931 μL, 5.33 mmol) and a solution of T3P in DCM (53% w/w, 960 mg, 1.60 mmol) were added dropwise. The mixture was allowed to warm up to ambient temperature and was stirred for 45 min. After completion, the reaction was quenched with the addition of a saturated aqueous solution of NH₄Cl at 0° C. Next, DCM was added. The aqueous layer was extracted with DCM. The combined organic layers were washed with a saturated aqueous solution of NH₄Cl, brine, dried over Na₂SO₄, filtered, and concentrated under reduced pressure. The crude was purified by flash column chromatography on silica using a gradient of EtOAc/EtOH (3/1) in n-heptane from 0% to 50%. Fractions were concentrated under reduced pressure to afford tert-butyl(4R)-5-amino-4-[5-[4-[(R)-(2-chlorophenyl)-phenyl-methyl]piperidine-1-carbonyl]-6-methyl-1-oxo-isoindolin-2-yl]-5-oxo-pentanoate as a white solid (565 mg, 99% p., 81% y.). m/z=[M+H]⁺644.5, ¹H NMR (400 MHz, DMSO-d₆): δ (ppm) 7.77-7.66 (m, 1H), 7.56 (t, J=6.3 Hz, 2H), 7.48-7.07 (m, 10H), 4.75-4.68 (m, 1H), 4.54 (d, J=15.4 Hz, 2H), 4.42 (dd, J=17.2, 7.4 Hz, 1H), 4.19-4.11 (m, 1H), 3.20 (d, J=17.5 Hz, 1H), 2.98 (q, J=13.5 Hz, 1H), 2.82-2.71 (m, 1H), 2.59 (d, J=12.1 Hz, 1H), 2.33 (d, J=4.1 Hz, 1H), 2.24 (s, 2H), 2.17-2.09 (m, 3H), 1.97 (t, J=7.5 Hz, 1H), 1.66-1.43 (m, 1H), 1.36-1.24 (m, 10H), 1.11 (t, J=11.8 Hz, 2H).

### Example 17

To a stirred solution of tert-butyl(4R)-5-amino-4-[5-[4-[(R)-(2-chlorophenyl)-phenyl-methyl]piperidine-1-carbonyl]-6-methyl-1-oxo-isoindolin-2-yl]-5-oxo-pentanoate (513 mg, 0.788 mmol) in acetonitrile (5.4 mL) was added 4-methylbenzenesulfonic acid hydrate (375 mg, 1.97 mmol). The reaction mixture was stirred at 70° C. for 18 h. The reaction mixture was cooled to ambient temperature. A saturated aqueous solution of NaHCO₃ and iPrOAc, were added to the reaction mixture. The layers were separated. The organic layer was washed with a saturated aqueous solution of NaHCO₃ (3×), with a saturated aqueous solution of NH₄Cl, brine, dried over Na₂SO₄, filtered, and concentrated under reduced pressure. The crude was co-evaporated with acetonitrile (2×) and was concentrated under reduced pressure to afford (3R)-3-[5-[4-[(R)-(2-chlorophenyl)-phenyl-methyl]piperidine-1-carbonyl]-6-methyl-1-oxo-isoindolin-2-yl]piperidine-2,6-dione as a white solid (369 mg, 95% p., 78% y.). m/z [M+H]⁺=570.4, ¹H NMR (400 MHz, DMSO-d₆): δ (ppm) 10.97 (s, 1H), 7.71 (d, J=18.2 Hz, 1H), 7.60 (s, 1H), 7.50-7.11 (m, 9H), 5.08 (s, 1H), 4.64-4.36 (m, 2H), 4.30 (d, J=16.9 Hz, 1H), 4.14 (d, J=11.1 Hz, 1H), 3.22 (s, 1H), 3.06-2.83 (m, 2H), 2.76 (s, 1H), 2.59 (d, J=15.5 Hz, 2H), 2.34 (s, 2H), 2.25 (s, 2H), 1.99 (s, 1H), 1.60 (s, 2H), 1.14 (s, 2H).

### Example 18

### (3S)-3-[3-[4-[(2-chlorophenyl)-phenyl-methyl]piperidine-1-carbonyl]-2-methyl-7-oxo-5H-pyrrolo[3,4-b]pyridin-6-yl]piperidine-2,6-dione

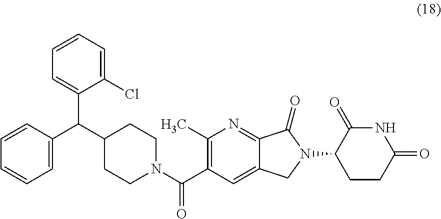 [SMILES: Cc1nc2c(cc1C(=O)N1CCC(C(c3ccccc3)c3ccccc3Cl)CC1)CN([C@H]1CCC(=O)NC1=O)C2=O]

### Intermediate 18A: tert-butyl(4S)-5-amino-4-[3-[4-[(2-chlorophenyl)-phenyl-methyl]piperidine-1-carbonyl]-2-methyl-7-oxo-5H-pyrrolo[3,4-b]pyridin-6-yl]-5-oxo-pentanoate

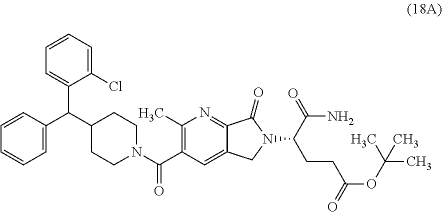 [SMILES: Cc1nc2c(cc1C(=O)N1CCC(C(c3ccccc3)c3ccccc3Cl)CC1)CN([C@@H](CCC(=O)OC(C)(C)C)C(N)=O)C2=O]

To a stirred suspension of 6-[(1S)-4-tert-butoxy-1-carbamoyl-4-oxo-butyl]-2-methyl-7-oxo-5H-pyrrolo[3,4-b]pyridine-3-carboxylic acid (52.0 mg, 0.138 mmol) in EtOAc (689 μL) were added successively 4-[(2-chlorophenyl)-phenyl-methyl]piperidine hydroiodide (59.9 mg, 0.145 mmol), DIPEA (120 μL, 0.689 mmol), and then a solution of T3P in MeTHF (50% w/w, 132 mg, 0.207 mmol). The reaction mixture was stirred at ambient temperature overnight. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by flash chromatography on silica gel using a gradient of EtOAc/EtOH (3/1) in cyclohexane from 2% to 100% to afford tert-butyl(4S)-5-amino-4-[3-[4-[(2-chlorophenyl)-phenyl-methyl]piperidine-1-carbonyl]-2-methyl-7-oxo-5H-pyrrolo[3,4-b]pyridin-6-yl]-5-oxo-pentanoate as a white solid (22.8 mg, 94% p., 24% y.). m/z [M+H]⁺=645.3.

### Example 18

To a stirred solution of tert-butyl(4S)-5-amino-4-[3-[4-[(2-chlorophenyl)-phenyl-methyl]piperidine-1-carbonyl]-2-methyl-7-oxo-5H-pyrrolo[3,4-b]pyridin-6-yl]-5-oxo-pentanoate (22.5 mg, 0.0349 mmol) in acetonitrile (349 μL) was added 4-methylbenzenesulfonic acid hydrate (33.2 mg, 0.174 mmol). The reaction mixture was stirred at 80° C. for 4 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure. The residue was basified with a saturated aqueous solution of NaHCO₃. The aqueous layer was extracted with EtOAc (3×). The combined organic layers were washed with brine, dried over Na₂SO₄, filtered, and concentrated under reduced pressure. The residue was solubilized in the minimum amount of DCM and the homogenous solution was added to a stirred solution of n-pentane dropwise. The resulting precipitate was triturated for 1 h, filtered, washed with n-pentane, and dried under reduced pressure at 50° C. overnight to afford (3S)-3-[3-[4-[(2-chlorophenyl)-phenyl-methyl]piperidine-1-carbonyl]-2-methyl-7-oxo-5H-pyrrolo[3,4-b]pyridin-6-yl]piperidine-2,6-dione as a white solid (8.0 mg, 98.6% p., 40% y.). m/z [M+H]⁺=571.3, ¹H NMR (600 MHz, DMSO-d₆): δ (ppm) 11.00 (br s, 1H), 8.01-7.82 (m, 1H), 7.78-7.64 (m, 1H), 7.42-7.10 (m, 8H), 5.19-5.10 (m, 1H), 4.60-4.25 (m, 3H), 4.15 (s, 1H), 3.29-2.72 (m, 4H), 2.65-2.52 (m, 3H), 2.45 (br s, 3H), 2.08-1.91 (m, 1H), 1.73-0.90 (m, 4H).

### Example 19

### (3S)-3-[5-[4-[(R)-(2-chloro-3-methoxy-phenyl)-phenyl-methyl]piperidine-1-carbonyl]-6-methyl-1-oxo-isoindolin-2-yl]piperidine-2,6-dione

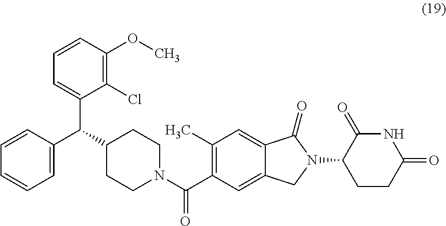 [SMILES: COc1cccc([C@@H](c2ccccc2)C2CCN(C(=O)c3cc4c(cc3C)C(=O)N([C@H]3CCC(=O)NC3=O)C4)CC2)c1Cl]

### Intermediate 19A: tert-butyl(4S)-5-amino-4-[5-[4-[(R)-(2-chloro-3-methoxy-phenyl)-phenyl-methyl]piperidine-1-carbonyl]-6-methyl-1-oxo-isoindolin-2-yl]-5-oxo-pentanoate

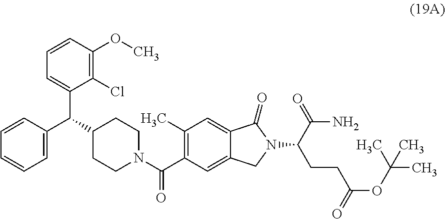 [SMILES: COc1cccc([C@@H](c2ccccc2)C2CCN(C(=O)c3cc4c(cc3C)C(=O)N([C@@H](CCC(=O)OC(C)(C)C)C(N)=O)C4)CC2)c1Cl]

To a stirred suspension of 4-[(R)-(2-chloro-3-methoxy-phenyl)-phenyl-methyl]piperidine hydroiodide (158 mg, 0.338 mmol) in EtOAc (2.1 mL) were added successively 2-[(1S)-4-tert-butoxy-1-carbamoyl-4-oxo-butyl]-6-methyl-1-oxo-isoindoline-5-carboxylic acid (125.0 mg, 0.327 mmol, DIPEA (295 μL, 1.69 mmol), and then a solution of T3P in EtOAc (50% w/w, 151 μL, 0.507 mmol). The reaction mixture was stirred at ambient temperature overnight. Additional solution of T3P in EtOAc (50% w/w, 151 μL, 0.507 mmol) was added and the reaction mixture was stirred at ambient temperature for 2 h. The reaction was quenched with the addition of water. FastWorX® powder was added. The organic solvent was evaporated under reduced pressure. Then, the reaction mixture was filtered through a loading cartridge and washed with an aqueous solution of NaHCO₃ and brine. The loading cartridge was flushed with nitrogen for 5 min and then loaded on a flash column chromatography. The crude was purified using a gradient of EtOAc/EtOH (3/1) in n-heptane from 0% to 100%. Fractions were concentrated under reduced pressure to afford tert-butyl(4S)-5-amino-4-[5-[4-[(R)-(2-chloro-3-methoxy-phenyl)-phenyl-methyl]piperidine-1-carbonyl]-6-methyl-1-oxo-isoindolin-2-yl]-5-oxo-pentanoate as an off-white solid (146 mg, 97% p., 62% y.). m/z [M+H]⁺=674.3. [α]_{D}=+10°·mL·dm⁻¹ g⁻¹[20° C., Na lamp, 589 nm, C=1.9 mg/mL, MeOH].

### Example 19

To a stirred solution of tert-butyl(4S)-5-amino-4-[5-[4-[(R)-(2-chloro-3-methoxy-phenyl)-phenyl-methyl]piperidine-1-carbonyl]-6-methyl-1-oxo-isoindolin-2-yl]-5-oxo-pentanoate (150 mg, 0.216 mmol) in acetonitrile (1.5 mL) at ambient temperature was added 4-methylbenzenesulfonic acid hydrate (123 mg, 0.649 mmol). The reaction mixture was stirred at 80° C. for 3 h. The reaction mixture was cooled to ambient temperature and diluted with water. The aqueous layer was extracted with EtOAc (2×). The combined organic layers were washed with brine, dried over Na₂SO₄, filtered, and concentrated under reduced pressure. The crude was purified by flash chromatography on silica gel using a gradient of EtOAc/EtOH (3/1) in cyclohexane from 10% to 100%. The residue was solubilized in a minimum amount of DCM and the homogenous solution was added to a stirred solution of n-pentane dropwise. The resulting precipitate was triturated for 1 h, filtered, washed with n-pentane, and dried under reduced pressure at 50° C. overnight to afford (3S)-3-[5-[4-[(R)-(2-chloro-3-methoxy-phenyl)-phenyl-methyl]piperidine-1-carbonyl]-6-methyl-1-oxo-isoindolin-2-yl]piperidine-2,6-dione as a white powder (48.6 mg, 99.8% p., 37% y.). m/z [M+H]⁺=600.3, ¹H NMR (500 MHz, DMSO-d₆): δ (ppm) 10.98 (s, 1H), 7.65-7.56 (m, 1H), 7.52-7.21 (m, 7H), 7.21-7.09 (m, 1H), 7.02-6.87 (m, 1H), 5.16-5.04 (m, 1H), 4.57-4.16 (m, 4H), 3.84-3.75 (m, 3H), 3.26 (br d, J=10.3 Hz, 1H), 3.07-2.68 (m, 3H), 2.65-2.54 (m, 2H), 2.44-2.20 (m, 4H), 2.06-1.93 (m, 1H), 1.74-0.82 (m, 4H).

### Example 20

### (3S)-3-[5-[4-[(S)-(2-chloro-3-methoxy-phenyl)-phenyl-methyl]piperidine-1-carbonyl]-6-methyl-1-oxo-isoindolin-2-yl]piperidine-2,6-dione

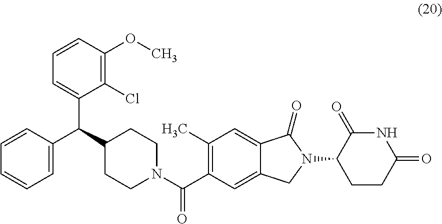 [SMILES: COc1cccc([C@H](c2ccccc2)C2CCN(C(=O)c3cc4c(cc3C)C(=O)N([C@H]3CCC(=O)NC3=O)C4)CC2)c1Cl]

### Intermediate 20A: tert-butyl(4S)-5-amino-4-[5-[4-[(S)-(2-chloro-3-methoxy-phenyl)-phenyl-methyl]piperidine-1-carbonyl]-6-methyl-1-oxo-isoindolin-2-yl]-5-oxo-pentanoate

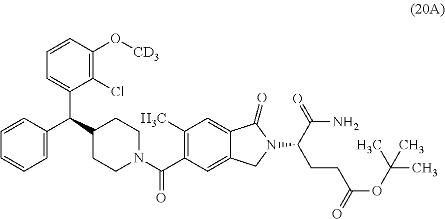 [SMILES: [2H]C([2H])([2H])Oc1cccc([C@H](c2ccccc2)C2CCN(C(=O)c3cc4c(cc3C)C(=O)N([C@@H](CCC(=O)OC(C)(C)C)C(N)=O)C4)CC2)c1Cl]

To a stirred suspension of 4-[(S)-(2-chloro-3-methoxy-phenyl)-phenyl-methyl]piperidine hydroiodide (150 mg, 0.338 mmol) in EtOAc (2.1 mL) were added successively 2-[(1S)-4-tert-butoxy-1-carbamoyl-4-oxo-butyl]-6-methyl-1-oxo-isoindoline-5-carboxylic acid (127 mg, 0.338 mmol), DIPEA (295 μL, 1.69 mmol), and then a solution of T3P in EtOAc (50% w/w, 151 μL, 0.507 mmol). The reaction mixture was stirred at ambient temperature overnight. Additional solution of T3P in EtOAc (50% w/w, 151 μL, 0.507 mmol) was added and the reaction mixture was stirred at ambient temperature for 2 h. The reaction was quenched with the addition of water. FastWorX® powder was added. The organic solvent was evaporated under reduced pressure. Then, the reaction mixture was filtered through a loading cartridge and washed with an aqueous solution of NaHCO₃ and brine. The loading cartridge was flushed with nitrogen for 5 min and then loaded on a flash column chromatography. The crude was purified using a gradient of EtOAc/EtOH (3/1) in n-heptane from 0% to 100%. Select fractions were concentrated under reduced pressure to afford tert-butyl(4S)-5-amino-4-[5-[4-[(S)-(2-chloro-3-methoxy-phenyl)-phenyl-methyl]piperidine-1-carbonyl]-6-methyl-1-oxo-isoindolin-2-yl]-5-oxo-pentanoate as an off-white solid (108 mg, 92% p., 44% y.). m/z [M+H]⁺=674.4. [α]_{D}=−38°·mL·dm⁻¹ g⁻¹[20° C., Na lamp, 589 nm, C=1.8 mg/mL, MeOH].

### Example 20

To a stirred solution of tert-butyl(4S)-5-amino-4-[5-[4-[(S)-(2-chloro-3-methoxy-phenyl)-phenyl-methyl]piperidine-1-carbonyl]-6-methyl-1-oxo-isoindolin-2-yl]-5-oxo-pentanoate (108 mg, 0.160 mmol) in acetonitrile (1.1 mL) at ambient temperature was added 4-methylbenzenesulfonic acid hydrate (91.3 mg, 0.480 mmol). The reaction mixture was stirred at 80° C. for 3 h. The reaction mixture was cooled to ambient temperature and diluted with water. The aqueous layer was extracted with EtOAc (2×). The combined organic layers were washed with brine, dried over Na₂SO₄, filtered, and concentrated under reduced pressure. The crude was purified by flash chromatography on silica gel using a gradient of a EtOAc/EtOH (3/1) in cyclohexane from 10% to 100%. The residue was solubilized in a minimum amount of DCM and the homogenous solution was added to a stirred solution of n-pentane dropwise. The resulting precipitate was triturated for 1 h, filtered, washed with n-pentane, and dried under reduced pressure at 50° C. overnight to afford (3S)-3-[5-[4-[(S)-(2-chloro-3-methoxy-phenyl)-phenyl-methyl]piperidine-1-carbonyl]-6-methyl-1-oxo-isoindolin-2-yl]piperidine-2,6-dione as a white powder (28.3 mg, 99.8% p., 29% y.). m/z [M+H]⁺=600.3, ¹H NMR (500 MHz, DMSO-d₆): δ (ppm) 10.97 (s, 1H), 7.65-7.57 (m, 1H), 7.50-7.10 (m, 8H), 7.01-6.86 (m, 1H), 5.17-5.03 (m, 1H), 4.58-4.48 (m, 1H), 4.48-4.36 (m, 1H), 4.36-4.16 (m, 2H), 3.86-3.74 (m, 3H), 3.29-3.13 (m, 1H), 3.06-2.84 (m, 2H), 2.83-2.70 (m, 1H), 2.65-2.54 (m, 2H), 2.47-2.22 (m, 4H), 2.05-1.92 (m, 1H), 1.69-0.85 (m, 4H).

### Example 21

### (3S)-3-[3-[4-[(2-chlorophenyl)-phenyl-methyl]piperidine-1-carbonyl]-2-methyl-7-oxo-5H-pyrrolo[3,4-b]pyrazin-6-yl]piperidine-2,6-dione

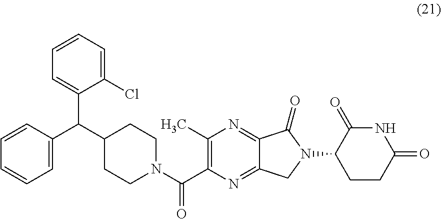 [SMILES: Cc1nc2c(nc1C(=O)N1CCC(C(c3ccccc3)c3ccccc3Cl)CC1)CN([C@H]1CCC(=O)NC1=O)C2=O]

### Intermediate 21A: tert-butyl(4S)-5-amino-4-[3-[4-[(2-chlorophenyl)-phenyl-methyl]piperidine-1-carbonyl]-2-methyl-7-oxo-5H-pyrrolo[3,4-b]pyrazin-6-yl]-5-oxo-pentanoate

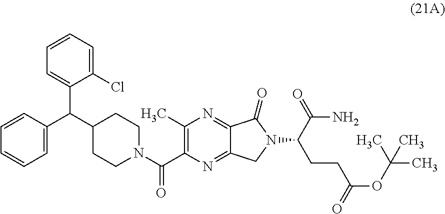 [SMILES: Cc1nc2c(nc1C(=O)N1CCC(C(c3ccccc3)c3ccccc3Cl)CC1)CN([C@@H](CCC(=O)OC(C)(C)C)C(N)=O)C2=O]

To a solution of 4-[(2-chlorophenyl)-phenyl-methyl]piperidine hydroiodide (70.0 mg, 0.169 mmol) in DMF (1.7 mL) were added 6-[(1S)-4-tert-butoxy-1-carbamoyl-4-oxo-butyl]-2-methyl-7-oxo-5H-pyrrolo[3,4-b]pyrazine-3-carboxylic acid (64 mg, 0.169 mmol), HATU (99.5 mg, 0.254 mmol), and DIPEA (148 μL, 0.846 mmol). The mixture was stirred at ambient temperature for 16 h. Water was added and the aqueous layer was extracted with EtOAc (2×). The organic layer was washed with water and brine, dried over Na₂SO₄, filtered, and concentrated. The crude was purified by flash chromatography on silica gel using a gradient of EtOAc/EtOH (3/1) in n-heptane from 5% to 50%. The desired fractions were combined, concentrated under reduced pressure, and dried under reduced pressure to afford tert-butyl(4S)-5-amino-4-[3-[4-[(2-chlorophenyl)-phenyl-methyl]piperidine-1-carbonyl]-2-methyl-7-oxo-5H-pyrrolo[3,4-b]pyrazin-6-yl]-5-oxo-pentanoate as an off-white powder (91.0 mg, 97% p., 81% y.). m/z [M+Na]⁺=668.4, ¹H NMR (400 MHz, DMSO-d₆): δ (ppm) 7.78-7.67 (m, 1H), 7.56 (s, 1H), 7.44-7.27 (m, 5H), 7.26-7.13 (m, 3H), 4.89-4.74 (m, 1H), 4.57-4.46 (m, 3H), 4.17 (d, J=11.2 Hz, 1H), 3.26 (d, J=2.9 Hz, 1H), 3.03 (q, J=13.2 Hz, 1H), 2.86 (q, J=12.0 Hz, 1H), 2.63 (d, J=11.4 Hz, 1H), 2.58-2.54 (m, 3H), 2.38-2.11 (m, 3H), 2.11-1.88 (m, 1H), 1.59 (dd, J=38.6, 13.0 Hz, 1H), 1.38 (d, J=13.1 Hz, 1H), 1.35-1.31 (m, 9H), 1.31-1.22 (m, 1H), 1.15 (dd, J=28.0, 14.4 Hz, 2H).

### Example 21

In a sealed vial, a solution of tert-butyl(4S)-5-amino-4-[3-[4-[(2-chlorophenyl)-phenyl-methyl]piperidine-1-carbonyl]-2-methyl-7-oxo-5H-pyrrolo[3,4-b]pyrazin-6-yl]-5-oxo-pentanoate (90.0 mg, 0.135 mmol) and 4-methylbenzenesulfonic acid hydrate (128 mg, 0.676 mmol) in acetonitrile (2.0 mL) was stirred at 70° C. for 7 h. The mixture was cooled to ambient temperature and poured into a saturated aqueous solution of NaHCO₃. The aqueous layer was extracted with EtOAc (2×). The combined organic layers were washed with brine, dried over Na₂SO₄, and concentrated. The crude was purified by flash column chromatography on silica gel using a gradient of EtOH in DCM from 0% to 5%. The desired fractions were concentrated under reduced pressure. The resulting residue was solubilized in a minimum amount of DCM and a large excess of n-pentane was added. The resulting suspension was filtered, washed with n-pentane, and dried under reduced pressure at 40° C. for 16 h to afford (3S)-3-[3-[4-[(2-chlorophenyl)-phenyl-methyl]piperidine-1-carbonyl]-2-methyl-7-oxo-5H-pyrrolo[3,4-b]pyrazin-6-yl]piperidine-2,6-dione as a white powder (41.8 mg, 97.5% p., 53% y.). m/z [M+H]⁺=572.3, ¹H NMR (600 MHz, DMSO-d₆): δ (ppm) 11.03 (s, 1H), 7.81-7.62 (m, 1H), 7.43-7.37 (m, 2H), 7.37-7.32 (m, 2H), 7.32-7.24 (m, 2H), 7.24-7.18 (m, 1H), 7.18-7.11 (m, 1H), 5.30-5.14 (m, 1H), 4.58-4.53 (m, 1H), 4.53-4.46 (m, 1H), 4.45-4.38 (m, 1H), 4.17 (dd, J=11.30, 2.64 Hz, 1H), 3.36-3.31 (m, 1H), 3.08-2.96 (m, 1H), 2.96-2.89 (m, 1H), 2.88-2.78 (m, 1H), 2.69-2.63 (m, 1H), 2.62-2.58 (m, 1H), 2.57 (dd, J=2.93, 1.91 Hz, 3H), 2.44 (br dd, J=13.13, 4.33 Hz, 1H), 2.07-1.97 (m, 1H), 1.70-1.50 (m, 1H), 1.44-1.26 (m, 1H), 1.22-1.10 (m, 1H), 1.10-0.99 (m, 1H).

### Example 22

### (3S)-3-[5-[4-[(2-chlorophenyl)-phenyl-methyl]piperidine-1-carbonyl]-6-[(dimethylamino)methyl]-1-oxo-isoindolin-2-yl]piperidine-2,6-dione

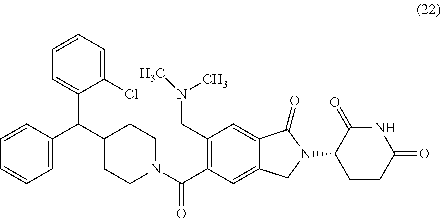 [SMILES: CN(C)Cc1cc2c(cc1C(=O)N1CCC(C(c3ccccc3)c3ccccc3Cl)CC1)CN([C@H]1CCC(=O)NC1=O)C2=O]

### Intermediate 22A: tert-butyl(4S)-5-amino-4-[5-[4-[(2-chlorophenyl)-phenyl-methyl]piperidine-1-carbonyl]-6-[(dimethylamino)methyl]-1-oxo-isoindolin-2-yl]-5-oxo-pentanoate

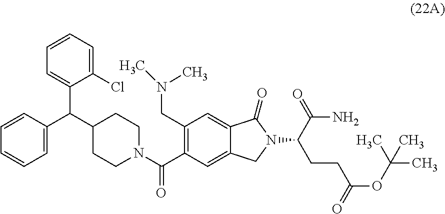 [SMILES: CN(C)Cc1cc2c(cc1C(=O)N1CCC(C(c3ccccc3)c3ccccc3Cl)CC1)CN([C@@H](CCC(=O)OC(C)(C)C)C(N)=O)C2=O]

To a stirred suspension of acetic acid 2-[(1S)-4-tert-butoxy-1-carbamoyl-4-oxo-butyl]-6-[(dimethylamino)methyl]-1-oxo-isoindoline-5-carboxylic acid (23.0 mg, 0.0480 mmol) in EtOAc (238 μL) and DCM (48 L) were added successively DIPEA (83.8 μL, 0.480 mmol), 4-[(2-chlorophenyl)-phenyl-methyl]piperidine hydroiodide (59.5 mg, 0.144 mmol), and then a solution of T3P in MeTHF (50% w/w, 76.3 mg, 0.120 mmol). The reaction mixture was stirred at ambient temperature overnight. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). Acetonitrile was removed under reduced pressure. The aqueous layer was basified with a saturated aqueous solution of NaHCO₃. The aqueous layer was extracted with EtOAc (3×). The combined organic layers were washed with brine, dried over Na₂SO₄, filtered, and concentrated under reduced pressure to afford tert-butyl(4S)-5-amino-4-[5-[4-[(2-chlorophenyl)-phenyl-methyl]piperidine-1-carbonyl]-6-[(dimethylamino)methyl]-1-oxo-isoindolin-2-yl]-5-oxo-pentanoate as an orange gum (42.3 mg, 29% p., 37% y.). m/z [M+H]⁺=687.3.

### Example 22

To a stirred solution of tert-butyl(4S)-5-amino-4-[5-[4-[(2-chlorophenyl)-phenyl-methyl]piperidine-1-carbonyl]-6-[(dimethylamino)methyl]-1-oxo-isoindolin-2-yl]-5-oxo-pentanoate (42.3 mg, 0.0615 mmol) in acetonitrile (620 μL) was added 4-methylbenzenesulfonic acid hydrate (58.5 mg, 0.308 mmol). The reaction mixture was stirred at 80° C. overnight. The reaction mixture was cooled to ambient temperature and quenched with a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by flash chromatography on silica gel using a gradient of EtOH in DCM from 0% to 20%. The residue was solubilized in the minimum amount of DCM and the homogenous solution was added to a stirred solution of n-pentane dropwise. The resulting precipitate was triturated for 1 h, filtered, washed with n-pentane, and dried under reduced pressure at 50° C. overnight to afford (3S)-3-[5-[4-[(2-chlorophenyl)-phenyl-methyl]piperidine-1-carbonyl]-6-[(dimethylamino)methyl]-1-oxo-isoindolin-2-yl]piperidine-2,6-dione as a white solid (1.1 mg, 99.4% p., 3% y.). m/z [M+H]⁺=613.3, ¹H NMR (600 MHz, DMSO-d₆): δ (ppm) 10.97 (d, J=3.8 Hz, 1H), 7.8-7.6 (m, 2H), 7.5-7.0 (m, 9H), 5.2-5.0 (m, 1H), 4.6-4.4 (m, 2H), 4.4-4.3 (m, 1H), 4.2-4.0 (m, 1H), 3.9-3.5 (m, 1H), 3.3-3.0 (m, 2H), 3.0-2.5 (m, 5H), 2.4-2.3 (m, 1H), 2.2-2.1 (m, 6H), 2.0-1.9 (m, 1H), 1.7-1.0 (m, 4H).

### Example 23

### [2-chloro-3-[[1-[2-[(3S)-2,6-dioxo-3-piperidyl]-6-methyl-1-oxo-isoindoline-5-carbonyl]-4-piperidyl]-phenyl-methyl]phenyl]boronic acid

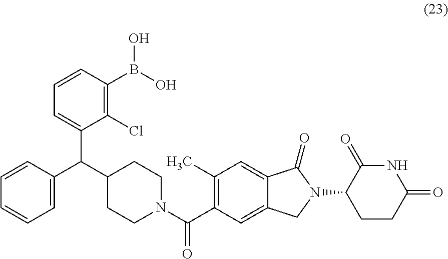 [SMILES: Cc1cc2c(cc1C(=O)N1CCC(C(c3ccccc3)c3cccc(B(O)O)c3Cl)CC1)CN([C@H]1CCC(=O)NC1=O)C2=O]

### Intermediate 23A: benzyl 4-[[2-chloro-3-(4,4,5,5-tetramethyl-1,3,2-dioxaborolan-2-yl)phenyl]-phenyl-methyl]piperidine-1-carboxylate

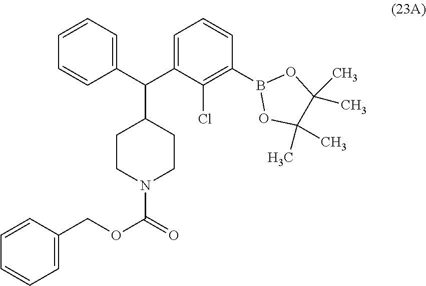 [SMILES: CC1(C)OB(c2cccc(C(c3ccccc3)C3CCN(C(=O)OCc4ccccc4)CC3)c2Cl)OC1(C)C]

To a stirred solution of benzyl 4-[(3-bromo-2-chloro-phenyl)-phenyl-methyl]piperidine-1-carboxylate (1.00 g, 1.86 mmol) in 1,4-dioxane (10 mL) at ambient temperature was added B₂pin₂(531 mg, 2.05 mmol) and KOAc (549 mg, 5.59 mmol). The reaction mixture was degassed with argon for 15 min and Pd(dppf)Cl₂ (140 mg, 0.186 mmol) was added. The reaction mixture was stirred at 100° C. for 2 h. The reaction mixture was filtered through a pad of talc and washed with EtOAc. A saturated aqueous solution of NH₄Cl was added to the filtrate. The layers were separated and the aqueous layer was extracted with EtOAc. The combined organic layers were washed with brine, dried using hydrophobic paper, and concentrated under reduced pressure to afford benzyl 4-[[2-chloro-3-(4,4,5,5-tetramethyl-1,3,2-dioxaborolan-2-yl)phenyl]-phenyl-methyl]piperidine-1-carboxylate as a brown gum (1.36 g, 80% p., quant. yield)). m/z [M+Na]⁺-568.3, ¹H NMR (400 MHz, DMSO-d₆): δ (ppm) 7.78 (dd, J=7.7, 1.9 Hz, 1H), 7.41-7.23 (m, 11H), 7.21-7.11 (m, 1H), 5.05 (s, 2H), 4.24-4.15 (m, 1H), 3.97 (d, J=13.3 Hz, 2H), 2.77 (m, 2H) 2.49 (s, 1H), 1.53-1.32 (m, 2H), 1.28 (s, 6H), 1.07 (s, 6H), 1.04-0.90 (m, 2H).

### Intermediate 23B: 4-[[2-chloro-3-(4,4,5,5-tetramethyl-1,3,2-dioxaborolan-2-yl)phenyl]-phenyl-methyl]piperidine hydroiodide

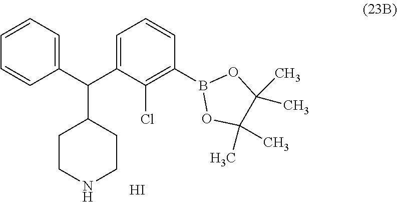

A round-bottom flask was charged with a suspension of benzyl 4-[[2-chloro-3-(4,4,5,5-tetramethyl-1,3,2-dioxaborolan-2-yl)phenyl]-phenyl-methyl]piperidine-1-carboxylate (200 mg, 0.293 mmol) in dry acetonitrile (3.7 mL) at ambient temperature under nitrogen. Iodo (trimethyl) silane (125 μL, 0.879 mmol) was added dropwise. The mixture was stirred at ambient temperature for 1 h. The mixture was cooled to 0° C. and MeOH (142 μL, 3.52 mmol) was added dropwise. The mixture was stirred at ambient temperature for 20 min and was concentrated under reduced pressure. Et₂O was added to the resulting residue. The reaction mixture was stirred at ambient temperature for 1 h. The solid was filtered, washed with Et₂O, and dried under reduced pressure at 50° C. for 16 h to afford 4-[[2-chloro-3-(4,4,5,5-tetramethyl-1,3,2-dioxaborolan-2-yl)phenyl]-phenyl-methyl]piperidine hydroiodide as a beige powder (192 mg, 72% p., 87% y.). m/z [M+H]⁺=412.4, ¹H NMR (400 MHz, DMSO-d₆): δ (ppm) 8.40 (d, J=11.6 Hz, 1H), 8.09 (d, J=11.3 Hz, 1H), 7.73 (dd, J=7.6, 1.9 Hz, 1H), 7.42-7.27 (m, 6H), 7.19 (dd, J=8.3, 6.1 Hz, 1H), 4.19 (d, J=11.2 Hz, 1H), 3.24 (d, J=12.4 Hz, 2H), 2.83 (p, J=11.7 Hz, 2H), 2.59 (d, J=11.5 Hz, 1H), 1.55 (t, J=15.7 Hz, 2H), 1.28 (s, 14H).

### Intermediate 23C: tert-butyl-(4S)-5-amino-4-[5-[4-[[2-chloro-3-(4,4,5,5-tetramethyl-1,3,2-dioxaborolan-2-yl)phenyl]-phenyl-methyl]piperidine-1-carbonyl]-6-methyl-1-oxo-isoindolin-2-yl]-5-oxo-pentanoate

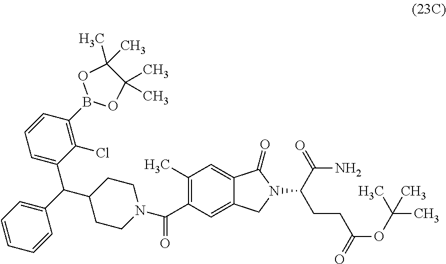 [SMILES: Cc1cc2c(cc1C(=O)N1CCC(C(c3ccccc3)c3cccc(B4OC(C)(C)C(C)(C)O4)c3Cl)CC1)CN([C@@H](CCC(=O)OC(C)(C)C)C(N)=O)C2=O]

In a round-bottom flask, to a solution of 2-[(1S)-4-tert-butoxy-1-carbamoyl-4-oxo-butyl]-6-methyl-1-oxo-isoindoline-5-carboxylic acid (147 mg, 0.391 mmol) in DCM (2.6 mL) was successively added 4-[[2-chloro-3-(4,4,5,5-tetramethyl-1,3,2-dioxaborolan-2-yl)phenyl]-phenyl-methyl]piperidine hydroiodide (192 mg, 0.356 mmol), a solution of T3P in EtOAc (50% w/w, 318 μL, 0.534 mmol), and DIPEA (249 μL, 1.42 mmol). The mixture was stirred at ambient temperature for 72 h. The organic layer was washed with water, with a saturated aqueous NaHCO₃, and then with brine. The organic layer was dried over Na₂SO₄, filtered, and concentrated under reduced pressure. The crude was purified by flash chromatography on silica gel using a gradient of EtOAc/EtOH (3/1) in cyclohexane from 0% to 100%. The desired fractions were combined and concentrated under reduced pressure to afford tert-butyl(4S)-5-amino-4-[5-[4-[[2-chloro-3-(4,4,5,5-tetramethyl-1,3,2-dioxaborolan-2-yl)phenyl]-phenyl-methyl]piperidine-1-carbonyl]-6-methyl-1-oxo-isoindolin-2-yl]-5-oxo-pentanoate as a brown solid (92.0 mg, 85% p., 29% y.). m/z [M+H]⁺=770.4, ¹H NMR (400 MHz, DMSO-d₆): δ (ppm) 7.79 (dd, J=28.9, 7.6 Hz, 1H), 7.56 (s, 2H), 7.49-7.08 (m, 9H), 4.75-4.68 (m, 1H), 4.54 (d, J=15.9 Hz, 2H), 4.41 (d, J=9.4 Hz, 1H), 4.23 (d, J=11.0 Hz, 1H), 3.22 (s, 1H), 3.17 (d, J=5.2 Hz, 1H), 3.06-2.90 (m, 1H), 2.76 (s, 1H), 2.58 (s, 1H), 2.33 (d, J=4.2 Hz, 1H), 2.23 (s, 2H), 2.14 (s, 3H), 2.07 (s, 1H), 1.97 (d, J=10.5 Hz, 1H), 1.55 (d, J=35.2 Hz, 1H), 1.37-1.00 (m, 22H).

### Example 23

In a sealed vial, to a solution of tert-butyl(4S)-5-amino-4-[5-[4-[[2-chloro-3-(4,4,5,5-tetramethyl-1,3,2-dioxaborolan-2-yl)phenyl]-phenyl-methyl]piperidine-1-carbonyl]-6-methyl-1-oxo-isoindolin-2-yl]-5-oxo-pentanoate (91.0 mg, 0.118 mmol) in acetonitrile (704 μL) was added 4-methylbenzenesulfonic acid hydrate (67.4 mg, 0.354 mmol). The solution was stirred at 90° C. for 2 h. The mixture was with EtOAc and washed with a saturated aqueous solution of NaHCO₃ and brine. The organic layer was dried over Na₂SO₄, filtered, and concentrated under reduced pressure. The crude was solubilized in DCM (1.0 mL) and treated with TFA (100 μL, 1.31 mmol) and methylboronic acid (70.7 mg, 1.18 mmol). The reaction mixture was stirred at ambient temperature for 2 h. The reaction mixture was concentrated under reduced pressure and the residue was purified by reverse-phase preparative chromatography using a gradient of acetonitrile in water from 0% to 100% (0.1% AcOH). The desired fractions were combined and concentrated under reduced pressure until acetonitrile was evaporated. The resulting suspension was filtered and dried under at 50° C. under reduced pressure overnight to afford [2-chloro-3-[1-[2-[(3S)-2,6-dioxo-3-piperidyl]-6-methyl-1-oxo-isoindoline-5-carbonyl]-4-piperidyl]-phenyl-methyl]phenyl]boronic acid as a white powder (26.7 mg, 99.3% p., 37% y.). m/z [M+H]⁺=614.2, ¹H NMR (600 MHz, DMSO-d₆): δ (ppm) 10.97 (br s, 1H), 8.30-8.15 (m, 2H), 7.74-7.57 (m, 2H), 7.52-7.43 (m, 1H), 7.41-7.32 (m, 3H), 7.32-7.20 (m, 3H), 7.19-7.15 (m, 1H), 7.13-7.09 (m, 1H), 5.13-5.05 (m, 1H), 4.62-4.09 (m, 4H), 3.29-3.17 (m, 1H), 3.03-2.85 (m, 2H), 2.83-2.71 (m, 1H), 2.63-2.54 (m, 2H), 2.42-2.30 (m, 2H), 2.25 (s, 2H), 2.02-1.94 (m, 1H), 1.68-1.37 (m, 1H), 1.34-0.90 (m, 2H).

### Example 24

### (3S)-3-[5-[4-[(S)-[2-chloro-3-[2-(dimethylamino) ethoxy]phenyl]-phenyl-methyl]piperidine-1-carbonyl]-6-methyl-1-oxo-isoindolin-2-yl]piperidine-2,6-dione hydrochloride

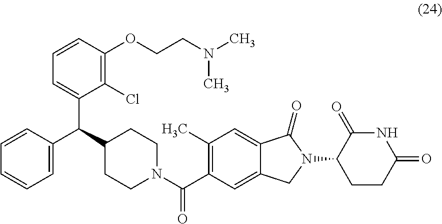 [SMILES: Cc1cc2c(cc1C(=O)N1CCC([C@@H](c3ccccc3)c3cccc(OCCN(C)C)c3Cl)CC1)CN([C@H]1CCC(=O)NC1=O)C2=O]

### Intermediate 24A: benzyl 4-[(S)-[2-chloro-3-[2-(dimethylamino) ethoxy]phenyl]-phenyl-methyl]piperidine-1-carboxylate

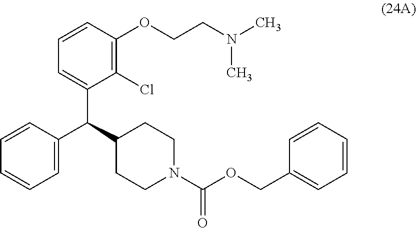 [SMILES: CN(C)CCOc1cccc([C@H](c2ccccc2)C2CCN(C(=O)OCc3ccccc3)CC2)c1Cl]

To a stirred solution of benzyl 4-[(S)-(2-chloro-3-hydroxy-phenyl)-phenyl-methyl]piperidine-1-carboxylate (250 mg, 0.568 mmol) in THF (2.8 mL) were added successively 2-(dimethylamino) ethanol (85.7 μL, 0.852 mmol), triphenylphosphane (223 mg, 0.852 mmol), and DIAD (171 μL, 0.852 mmol). The reaction mixture was stirred at ambient temperature overnight. The reaction mixture was concentrated under reduced pressure. The crude was purified by flash chromatography on silica gel using a gradient of MeOH in DCM from 1% to 20% to afford (benzyl 4-[(S)-[2-chloro-3-[2-(dimethylamino) ethoxy]phenyl]-phenyl-methyl]piperidine-1-carboxylate as a light yellow solid (200 mg, 78% p., 54% y.). m/z [M+H]⁺=507.4, ¹H NMR (400 MHz, DMSO-d₆): δ (ppm) 7.40-7.22 (m, 11H), 7.20-7.11 (m, 1H), 6.95 (dd, J=6.2, 3.4 Hz, 1H), 5.05 (s, 2H), 4.16 (d, J=11.2 Hz, 1H), 4.06 (qt, J=10.2, 5.6 Hz, 2H), 4.00-3.90 (m, 2H), 2.78 (s, 2H), 2.63 (t, J=5.7 Hz, 2H), 2.22 (s, 6H), 1.47 (d, J=13.1 Hz, 1H), 1.36 (dd, J=14.8, 9.6 Hz, 1H), 1.28-1.17 (m, 1H), 1.00 (dqd, J=24.7, 12.4, 4.1 Hz, 2H), [α]_{D}=−49°·mL·dm-1.g⁻¹[20° C., Na lamp, 589 nm, C=1.9 mg/mL, EtOH].

### Intermediate 24B: 2-[2-chloro-3-[(S)-phenyl(4-piperidyl)methyl]phenoxy]-N,N-dimethyl-ethanamine dihydroiodide

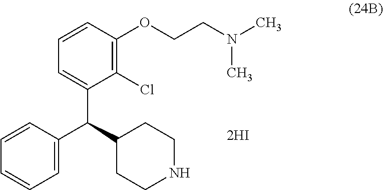 [SMILES: CN(C)CCOc1cccc([C@H](c2ccccc2)C2CCNCC2)c1Cl]

To a stirred solution of benzyl 4-[(S)-[2-chloro-3-[2-(dimethylamino) ethoxy]phenyl]-phenyl-methyl]piperidine-1-carboxylate (200 mg, 0.331 mmol) in dry anhydrous acetonitrile (3.3 mL) at ambient temperature under nitrogen was added iodo (trimethyl) silane (141 μL, 0.994 mmol) dropwise. The reaction mixture was stirred at ambient temperature for 1 h. MeOH (161 μL, 3.98 mmol) was added dropwise. The mixture was stirred at ambient temperature for 2 h and was concentrated under reduced pressure. Et₂O was added and the resulting mixture was stirred at ambient temperature for 1 h. The solid was filtered, washed with Et₂O, and dried under reduced pressure to afford (2-[2-chloro-3-[(S)-phenyl(4-piperidyl)methyl]phenoxy]-N,N-dimethyl-ethanamine dihydroiodide as a yellow powder (282 mg, 56% p., 76% y.). m/z [M+H]⁺=373.4, ¹H NMR (400 MHz, DMSO-d₆): δ (ppm) 9.45 (d, J=34.9 Hz, 1H), 8.41 (s, 1H), 8.08 (s, 1H), 7.40-7.24 (m, 5H), 7.23-7.15 (m, 1H), 7.04 (dd, J=7.8, 1.7 Hz, 1H), 4.45-4.23 (m, 2H), 4.15 (d, J=11.2 Hz, 1H), 3.53 (s, 2H), 3.23 (d, J=11.5 Hz, 2H), 2.85 (d, J=35.3 Hz, 8H), 2.61 (s, 1H), 1.55 (dd, J=31.9, 14.1 Hz, 2H), 1.23 (q, J=8.8 Hz, 4H), [α]_{D}=−24°·mL·dm⁻¹ g⁻¹[20° C., Na lamp, 589 nm, C=1.3 mg/mL, EtOH].

### Intermediate 24C: tert-butyl(4S)-5-amino-4-[5-[4-[(S)-[2-chloro-3-[2-(dimethylamino) ethoxy]phenyl]-phenyl-methyl]piperidine-1-carbonyl]-6-methyl-1-oxo-isoindolin-2-yl]-5-oxo-pentanoate

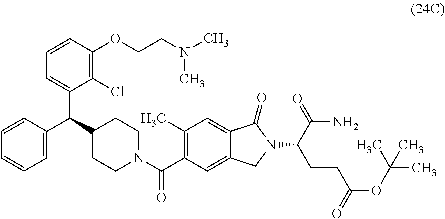 [SMILES: Cc1cc2c(cc1C(=O)N1CCC([C@@H](c3ccccc3)c3cccc(OCCN(C)C)c3Cl)CC1)CN([C@@H](CCC(=O)OC(C)(C)C)C(N)=O)C2=O]

To a solution of 2-[2-chloro-3-[(S)-phenyl(4-piperidyl)methyl]phenoxy]-N,N-dimethyl-ethanamine dihydroiodide (158 mg, 0.251 mmol) in DMF (692 μL) were added 2-[(1S)-4-tert-butoxy-1-carbamoyl-4-oxo-butyl]-6-methyl-1-oxo-isoindoline-5-carboxylic acid (90.0 mg, 0.239 mmol), DIPEA (209 μL, 1.20 mmol), and a 0.5 M solution of HATU in DMF (717 μL, 0.359 mmol). The reaction mixture was stirred at ambient temperature for 2 h. Water, EtOAc and FastWorkX® were added. The organic solvent was removed under reduced pressure. The reaction mixture was filtered through a dry-loading cartridge and the resin was rinsed with water. The crude was purified by reverse-phase preparative chromatography using a gradient of acetonitrile in water from 0% to 100% (0.1% AcOH). The desired fractions were combined, and the acetonitrile was evaporated under reduced pressure. The resulting aqueous layer was basified with a saturated aqueous solution of NaHCO₃(until pH=9) and the aqueous layer was extracted with DCM (3×). The combined organic layers were washed with brine, dried using a phase separator cartridge and concentrated under reduced pressure to afford tert-butyl(4S)-5-amino-4-[5-[4-[(S)-[2-chloro-3-[2-(dimethylamino) ethoxy]phenyl]-phenyl-methyl]piperidine-1-carbonyl]-6-methyl-1-oxo-isoindolin-2-yl]-5-oxo-pentanoate as a colorless solid (40.0 mg, 97% p., 22% y.). m/z [M+H]⁺=731.4. [α]_{D}=−29°·mL·dm⁻¹ g⁻¹[20° C., Na lamp, 589 nm, C=1.5 mg/mL, EtOH].

### Example 24

To a stirred solution of tert-butyl(4S)-5-amino-4-[5-[4-[(S)-[2-chloro-3-[2-(dimethylamino) ethoxy]phenyl]-phenyl-methyl]piperidine-1-carbonyl]-6-methyl-1-oxo-isoindolin-2-yl]-5-oxo-pentanoate (40.0 mg, 0.0531 mmol) in acetonitrile (530 μL) was added 4-methylbenzenesulfonic acid hydrate (50.5 mg, 0.265 mmol). The reaction mixture was stirred at 80° C. for 4 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure. The residue was solubilized in 1,4-dioxane (1.4 mL). A 4 M solution of HCl in 1,4-dioxane (136 μL) was added and the reaction mixture was stirred for 30 min. Et₂O (14 mL) was added dropwise and the reaction mixture was stirred overnight. The resulting precipitate was filtered, washed with Et₂O, and dried under reduced pressure at 50° C. overnight to afford (3S)-3-[5-[4-[(S)-[2-chloro-3-[2-(dimethylamino) ethoxy]phenyl]-phenyl-methyl]piperidine-1-carbonyl]-6-methyl-1-oxo-isoindolin-2-yl]piperidine-2,6-dione hydrochloride as a white powder (11.5 mg, 99.5% p. 31% y.). m/z [M+H]⁺=657.4, ¹H NMR (600 MHz, DMSO-d₆): δ (ppm) 10.97 (br d, J=10.1 Hz, 1H), 9.94 (br s, 1H), 7.61 (br d, J=6.2 Hz, 1H), 7.50-7.21 (m, 7H), 7.20-7.10 (m, 1H), 7.04-6.95 (m, 1H), 5.15-5.04 (m, 1H), 4.65-4.10 (m, 6H), 3.55-3.38 (m, 2H), 3.26-2.66 (m, 10H), 2.62-2.54 (m, 2H), 2.44-2.20 (m, 4H), 2.05-1.92 (m, 1H), 1.68-1.28 (m, 2H), 1.21-0.89 (m, 2H), [α]_{D}=−32°·mL·dm⁻¹ g⁻¹[20° C., Na lamp, 589 nm, C=1.1 mg/mL, EtOH].

### Example 25

### (3S)-3-[5-[4-[(S)-[2-chloro-3-(2-hydroxyethoxy)phenyl]-phenyl-methyl]piperidine-1-carbonyl]-6-methyl-1-oxo-isoindolin-2-yl]piperidine-2,6-dione

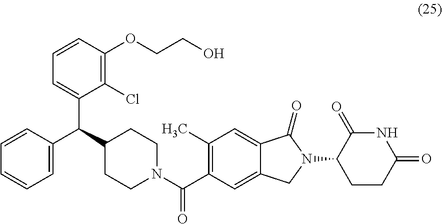 [SMILES: Cc1cc2c(cc1C(=O)N1CCC([C@@H](c3ccccc3)c3cccc(OCCO)c3Cl)CC1)CN([C@H]1CCC(=O)NC1=O)C2=O]

### Intermediate 25A: benzyl 4-[(S)-[3-[2-[tert-butyl(dimethyl) silyl]oxyethoxy]-2-chloro-phenyl]-phenyl-methyl]piperidine-1-carboxylate

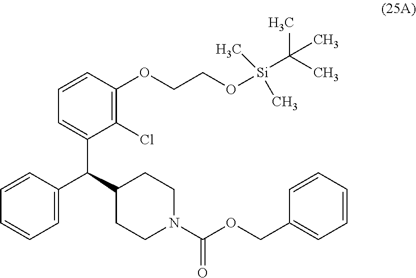 [SMILES: CC(C)(C)[Si](C)(C)OCCOc1cccc([C@H](c2ccccc2)C2CCN(C(=O)OCc3ccccc3)CC2)c1Cl]

To a stirred solution of benzyl 4-[(S)-(2-chloro-3-hydroxy-phenyl)-phenyl-methyl]piperidine-1-carboxylate (250 mg, 0.568 mmol) in THF (2.8 mL) were added successively 2-[tert-butyl(dimethyl) silyl]oxyethanol (158 mg, 0.852 mmol), triphenylphosphane (223 mg, 0.852 mmol), and then DIAD (171 μL, 0.852 mmol). The reaction mixture was stirred at ambient temperature overnight. The reaction mixture was concentrated under reduced pressure. The crude was purified by flash chromatography on silica gel using a gradient EtOAc in cyclohexane from 1% to 30% to afford (benzyl 4-[(S)-[3-[2-[tert-butyl(dimethyl) silyl]oxyethoxy]-2-chlorophenyl]-phenyl-methyl]piperidine-1-carboxylate as a colorless oil (332 mg, 97% p., 95% y.). m/z [M+H]⁺=594.4. [α]_{D}=−41°·mL·dm⁻¹.g⁻¹[20° C., Na lamp, 589 nm, C=2.2 mg/mL, EtOH].

### Intermediate 25B: 2-[2-chloro-3-[(S)-phenyl(4-piperidyl)methyl]phenoxy]ethanol hydrochloride

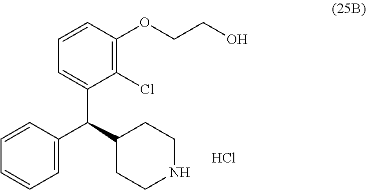 [SMILES: OCCOc1cccc([C@H](c2ccccc2)C2CCNCC2)c1Cl ; Cl]

To a stirred solution of benzyl 4-[(S)-[3-[2-[tert-butyl(dimethyl) silyl]oxyethoxy]-2-chloro-phenyl]-phenyl-methyl]piperidine-1-carboxylate (340 mg, 0.555 mmol) in dry acetonitrile (5.6 mL) at ambient temperature under nitrogen was added iodo (trimethyl) silane (237 μL, 1.67 mmol) dropwise. The reaction mixture was stirred at ambient temperature for 3 h. The mixture was cooled to 0° C. and MeOH (270 μL, 6.66 mmol) was added dropwise. The mixture was stirred at ambient temperature for 30 min and was concentrated under reduced pressure. The crude was purified by reverse-phase preparative chromatography using a gradient of acetonitrile in water from 0% to 100% (0.1% AcOH in water). The desired fractions were concentrated under reduced pressure. The residue was solubilized in 1,4-dioxane (1.6 mL) and a 4 M solution of HCl (1.6 mL) was added dropwise. The reaction mixture was stirred for 30 min and Et₂O (16 mL) was added dropwise. The resulting precipitate formed was triturated for 1 h, filtered, washed with Et₂O, and dried under reduced pressure overnight to afford 2-[2-chloro-3-[(S)-phenyl(4-piperidyl)methyl]phenoxy]ethanol hydrochloride as a yellow solid (150 mg, 97% p., 69% y.). m/z [M+H]⁺=346.3. [α]_{D}=−66°·mL·dm⁻¹ g⁻¹[20° C., Na lamp, 589 nm, C=1.70 mg/mL, EtOH].

### Intermediate 25C: tert-butyl(4S)-5-amino-4-[5-[4-[(S)-[2-chloro-3-(2-hydroxyethoxy)phenyl]-phenyl-methyl]piperdine-1-carbonyl]-6-methyl-1-oxo-isoindolin-2-yl]-5-oxo-pentanoate

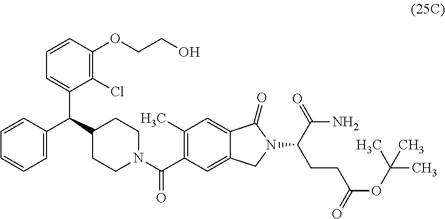 [SMILES: Cc1cc2c(cc1C(=O)N1CCC([C@@H](c3ccccc3)c3cccc(OCCO)c3Cl)CC1)CN([C@@H](CCC(=O)OC(C)(C)C)C(N)=O)C2=O]

To a solution of 2-[2-chloro-3-[(S)-phenyl(4-piperidyl)methyl]phenoxy]ethanol hydrochloride (148 mg, 0.377 mmol) in DMF (1.2 mL) were added 2-[(1S)-4-tert-butoxy-1-carbamoyl-4-oxo-butyl]-6-methyl-1-oxo-isoindoline-5-carboxylic acid (135 mg, 0.359 mmol), DIPEA (313 μL, 1.79 mmol), and a 0.5 M solution of HATU in DMF (1.1 mL, 0.538 mmol). The reaction mixture was stirred at room temperature for 2 h. Water, EtOAc, and FastWorkX® were added. The organic solvent was removed under reduced pressure. The reaction mixture was filtered through a dry-loading cartridge and the resin was rinsed with water. The crude was purified by flash column chromatography on silica using a gradient of EtOAc/EtOH (3/1) (with 5% of conc. NH₄OH) in cyclohexane from 5% to 100%. The desired fractions were combined and concentrated under reduced pressure to afford tert-butyl(4S)-5-amino-4-[5-[4-[(S)-[2-chloro-3-(2-hydroxyethoxy)phenyl]-phenyl-methyl]piperidine-1-carbonyl]-6-methyl-1-oxo-isoindolin-2-yl]-5-oxo-pentanoate as a white solid (180 mg, 90% p., 64% y.). m/z [M+H]⁺=704.3. [α]_{D}=−34°·mL·dm⁻¹.g⁻¹[20° C., Na lamp, 589 nm, C=1.4 mg/mL, EtOH].

### Example 25

To a stirred solution of tert-butyl(4S)-5-amino-4-[5-[4-[(S)-[2-chloro-3-(2-hydroxyethoxy)phenyl]-phenyl-methyl]piperidine-1-carbonyl]-6-methyl-1-oxo-isoindolin-2-yl]-5-oxo-pentanoate (180 mg, 0.256 mmol) in acetonitrile (2.6 mL) was added 4-methylbenzenesulfonic acid hydrate (243 mg, 1.28 mmol) The reaction mixture was stirred at 80° C. for 5 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure. The residue was basified with a saturated aqueous solution of NaHCO₃. The aqueous layer was extracted with EtOAc (3×). The combined organic layers were washed with brine, dried over Na₂SO₄, filtered, and concentrated under reduced pressure. The residue was solubilized in the minimum amount of DCM and the homogenous solution was added to a stirred solution of n-pentane dropwise. The resulting precipitate was triturated for 1 h, filtered, washed with n-pentane, and dried under reduced pressure at 50° C. overnight to afford (3S)-3-[5-[4-[(S)-[2-chloro-3-(2-hydroxyethoxy)phenyl]-phenyl-methyl]piperidine-1-carbonyl]-6-methyl-1-oxo-isoindolin-2-yl]piperidine-2,6-dione as a white powder (55.8 mg, 96.6% p., 33% y.). m/z [M+H]⁺=630.3, ¹H NMR (500 MHz, DMSO-d₆): δ (ppm) 11.20-10.75 (m, 1H), 7.64-7.57 (m, 1H), 7.54-7.20 (m, 7H), 7.21-7.09 (m, 1H), 7.02-6.89 (m, 1H), 5.17-5.02 (m, 1H), 4.90-4.75 (m, 1H), 4.59-4.48 (m, 1H), 4.47-4.36 (m, 1H), 4.35-4.26 (m, 1H), 4.26-4.17 (m, 1H), 4.14-3.85 (m, 2H), 3.80-3.62 (m, 2H), 3.29-3.14 (m, 1H), 3.09-2.69 (m, 3H), 2.66-2.54 (m, 2H), 2.46-2.20 (m, 4H), 2.06-1.92 (m, 1H), 1.71-0.83 (m, 4H), [α]_{D}=−47°·mL·dm⁻¹ g⁻¹[20° C., Na lamp, 589 nm, C=1.7 mg/mL, EtOH].

### Example 26

### (3S)-3-[5-[4-[(R)-[2-chloro-3-[2-(dimethylamino) ethoxy]phenyl]-phenyl-methyl]piperidine-1-carbonyl]-6-methyl-1-oxo-isoindolin-2-yl]piperidine-2,6-dione hydrochloride

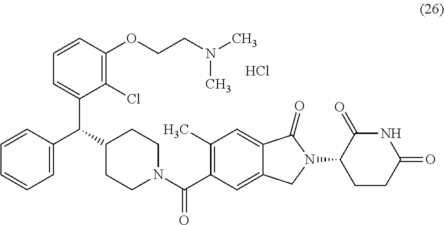 [SMILES: Cc1cc2c(cc1C(=O)N1CCC([C@H](c3ccccc3)c3cccc(OCCN(C)C)c3Cl)CC1)CN([C@H]1CCC(=O)NC1=O)C2=O ; Cl]

### Intermediate 26A: benzyl 4-[(R)-[2-chloro-3-[2-(dimethylamino) ethoxy]phenyl]-phenyl-methyl]piperidine-1-carboxylate

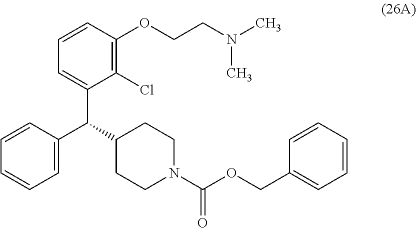 [SMILES: CN(C)CCOc1cccc([C@@H](c2ccccc2)C2CCN(C(=O)OCc3ccccc3)CC2)c1Cl]

In a sealed vial, to a stirred solution of benzyl 4-[(R)-(2-chloro-3-hydroxy-phenyl)-phenyl-methyl]piperidine-1-carboxylate (250 mg, 0.573 mmol) in THF (2.9 mL) were added successively 2-(dimethylamino) ethanol (86.5 L, 0.860 mmol), triphenylphosphane (226 mg, 0.860 mmol), and then DIAD (169 μL, 0.860 mmol). The reaction mixture was stirred at ambient temperature overnight. The reaction mixture was concentrated under reduced pressure. The crude was purified by flash chromatography on silica gel using a gradient of EtOAc/EtOH (3/1) in cyclohexane from 1% to 100%. The desired fractions were combined and concentrated under reduced pressure to afford benzyl 4-[(R)-[2-chloro-3-[2-(dimethylamino) ethoxy]phenyl]-phenyl-methyl]piperidine-1-carboxylate as a colorless oil (297 mg, 94% p., 96% y.). m/z [M+H]⁺=507.3, ¹H NMR (400 MHz, DMSO-d₆): δ (ppm) 7.34 (dddd, J=10.2, 6.7, 3.7, 2.2 Hz, 7H), 7.29-7.22 (m, 4H), 7.19-7.14 (m, 1H), 6.95 (dd, J=6.2, 3.4 Hz, 1H), 5.05 (s, 2H), 4.16 (d, J=11.2 Hz, 1H), 4.06 (qt, J=10.1, 5.7 Hz, 2H), 3.96 (d, J=13.1 Hz, 2H), 2.78 (s, 3H), 2.22 (s, 6H), 2.13 (s, 2H), 1.47 (d, J=13.1 Hz, 1H), 1.42-1.31 (m, 1H), 1.00 (ddd, J=24.7, 12.3, 3.9 Hz, 2H), [α]_{D}=+53°·mL·dm⁻¹ g⁻¹[20° C., Na lamp, 589 nm, C=1.3 mg/mL, MeOH].

### Intermediate 26B: 2-[2-chloro-3-[(R)-phenyl(4-piperidyl)methyl]phenoxy]-N,N-dimethyl-ethanamine dihydroiodide

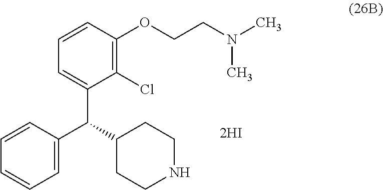 [SMILES: CN(C)CCOc1cccc([C@@H](c2ccccc2)C2CCNCC2)c1Cl]

In a round-bottom flask, to a stirred solution of benzyl 4-[(R)-[2-chloro-3-[2-(dimethylamino) ethoxy]phenyl]-phenyl-methyl]piperidine-1-carboxylate (313 mg, 0.580 mmol) in dry acetonitrile (5.8 mL) at ambient temperature under nitrogen was added iodo (trimethyl) silane (248 μL, 1.74 mmol) dropwise. The reaction mixture was stirred at ambient temperature for 1 h. The mixture was cooled to 0° C. and MeOH (282 μL, 6.96 mmol) was added dropwise. The mixture was stirred at ambient temperature for 1 h and was concentrated under reduced pressure. Et₂O was added and the resulting mixture was stirred at ambient temperature for 1 h. The solid was filtered, washed with Et₂O, and dried under reduced pressure at 50° C. to afford 2-[2-chloro-3-[(R)-phenyl(4-piperidyl)methyl]phenoxy]-N,N-dimethyl-ethanamine dihydroiodide as a yellow solid (424 mg, 67% p., 78% y.). m/z [M+H]⁺=373.4. [α]_{D}=+31°·mL·dm⁻¹ g⁻¹[20° C., Na lamp, 589 nm, C=1.6 mg/mL, MeOH].

### Intermediate 26C: tert-butyl(4S)-5-amino-4-[5-[4-[(R)-[2-chloro-3-[2-(dimethylamino) ethoxy]phenyl]-phenyl-methyl]piperidine-1-carbonyl]-6-methyl-1-oxo-isoindolin-2-yl]-5-oxo-pentanoate

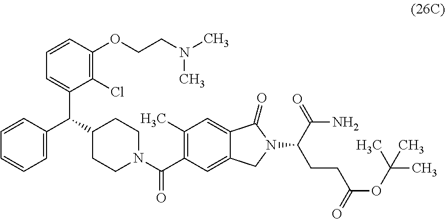 [SMILES: Cc1cc2c(cc1C(=O)N1CCC([C@H](c3ccccc3)c3cccc(OCCN(C)C)c3Cl)CC1)CN([C@@H](CCC(=O)OC(C)(C)C)C(N)=O)C2=O]

In a round-bottom flask, to a stirred suspension of 2-[(1S)-4-tert-butoxy-1-carbamoyl-4-oxo-butyl]-6-methyl-1-oxo-isoindoline-5-carboxylic acid (160 mg, 0.425 mmol) in DMF (2.0 mL) were added successively 2-[2-chloro-3-[(R)-phenyl(4-piperidyl)methyl]phenoxy]-N,N-dimethyl-ethanamine dihydroiodide (420 mg, 0.448 mmol), DIPEA (371 μL, 2.13 mmol) and then a 0.5 M solution of HATU in DMF (1.3 mL, 0.638 mmol). The reaction mixture was stirred at ambient temperature for 1 h. The reaction was quenched with the addition of water and EtOAc. Next, FastWorX® powder were added.

The organic layer was evaporated under reduced pressure. The reaction mixture was filtered through a Combiflash loading cartridge and the reactor was rinsed with water. The crude was purified by reverse-phase preparative chromatography using a gradient of acetonitrile in water from 0% to 100% (0.1% AcOH). The desired fractions were combined and concentrated under reduced pressure until acetonitrile evaporated. A saturated aqueous solution of NaHCO₃ and DCM were added. The layers were separated and the aqueous layer was extracted with DCM (2×). The combined organic layers were washed with brine, dried using a phase separator cartridge and concentrated under reduced pressure to afford tert-butyl(4S)-5-amino-4-[5-[4-[(R)-[2-chloro-3-[2-(dimethylamino) ethoxy]phenyl]-phenyl-methyl]piperidine-1-carbonyl]-6-methyl-1-oxo-isoindolin-2-yl]-5-oxo-pentanoate as a pale yellow foam (122 mg, 100% p., 39% y.). m/z [M+H]⁺=731.4. [α]_{D}=+15°·mL·dm⁻¹ g⁻¹[20° C., Na lamp, 589 nm, C=1.2 mg/mL, MeOH].

### Example 26

To a stirred solution of tert-butyl(4S)-5-amino-4-[5-[4-[(R)-[2-chloro-3-[2-(dimethylamino) ethoxy]phenyl]-phenyl-methyl]piperidine-1-carbonyl]-6-methyl-1-oxo-isoindolin-2-yl]-5-oxo-pentanoate (118 mg, 0.161 mmol) in acetonitrile (1.6 mL) was added 4-methylbenzenesulfonic acid hydrate (153 mg, 0.807 mmol). The reaction mixture was stirred at 80° C. for 4 h. The reaction mixture was cooled to ambient temperature and quenched with a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure. The residue was solubilized in 1,4-dioxane (4.1 mL). A 4 M solution HCl in 1,4-dioxane (413 μL) was added dropwise and the reaction mixture was stirred for 30 min. Et₂O (41 mL) was added dropwise and the reaction mixture was stirred for 1 h. The resulting precipitate was filtered, washed with Et₂O, and dried under reduced pressure at 50° C. overnight to afford (3S)-3-[5-[4-[(R)-[2-chloro-3-[2-(dimethylamino) ethoxy]phenyl]-phenyl-methyl]piperidine-1-carbonyl]-6-methyl-1-oxo-isoindolin-2-yl]piperidine-2,6-dione hydrochloride as a white powder (58.5 mg, 98.7% p., 52% y.). m/z [M+H]⁺=657.4, ¹H NMR (600 MHz, DMSO-d₆): δ (ppm) 10.99-10.95 (m, 1H), 10.05 (br s, 1H), 7.60 (d, J=7.4 Hz, 1H), 7.49-7.26 (m, 6H), 7.24 (br t, J=7.4 Hz, 1H), 7.20-7.10 (m, 1H), 7.05-6.95 (m, 1H), 5.15-5.04 (m, 1H), 4.59-4.13 (m, 6H), 3.55-3.41 (m, 2H), 3.28-2.70 (m, 10H), 2.62-2.56 (m, 2H), 2.41-2.22 (m, 4H), 2.02-1.96 (m, 1H), 1.67-1.24 (m, 2H), 1.19-0.89 (m, 2H), [α]_{D}=+8°·mL·dm⁻¹ g⁻¹[20° C., Na lamp, 589 nm, C=1.5 mg/mL, EtOH].

### Example 27

### (3S)-3-[5-[4-[[2-chloro-3-(methylamino)phenyl]-phenyl-methyl]piperidine-1-carbonyl]-6-methyl-1-oxo-isoindolin-2-yl]piperidine-2,6-dione

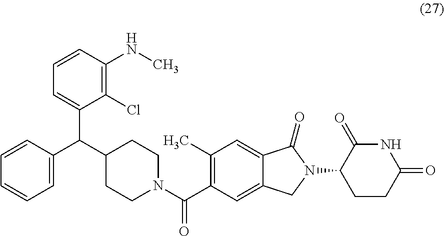 [SMILES: CNc1cccc(C(c2ccccc2)C2CCN(C(=O)c3cc4c(cc3C)C(=O)N([C@H]3CCC(=O)NC3=O)C4)CC2)c1Cl]

### Intermediate 27A: benzyl 4-[[2-chloro-3-(methylamino)phenyl]-phenyl-methyl]piperidine-1-carboxylate

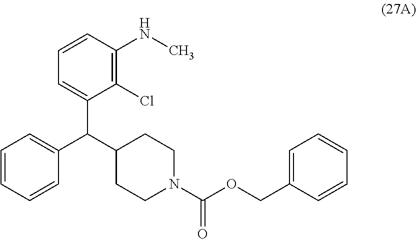 [SMILES: CNc1cccc(C(c2ccccc2)C2CCN(C(=O)OCc3ccccc3)CC2)c1Cl]

In a sealed vial, to a stirred solution of benzyl 4-[(3-bromo-2-chloro-phenyl)-phenyl-methyl]piperidine-1-carboxylate (150 mg, 0.280 mmol) in tert-butanol (1.1 mL) under argon were added successively t-BuONa (32.3 mg, 0.336 mmol), a 2 M solution of methanamine in MeOH (7.0 mL, 14.0 mmol) and Brettphos-Pd-G1 (23.5 mg, 0.0280 mmol) and the mixture was stirred at 80° C. for 23 h. EtOAc was added and the mixture was washed with a saturated aqueous solution of NH₄Cl, water, brine, dried over Na₂SO₄, filtered, and concentrated under reduced pressure. The crude was purified by flash chromatography on silica gel using a gradient of EtOAc in n-heptane from 0% to 50%. Fractions were combined and concentrated under reduced pressure to afford a mixture of expected compound and starting material as a colorless gum (145 mg). This mixture was solubilized in tert-butanol (1.1 mL) and t-BuONa (32.3 mg, 0.336 mmol), a 2 M solution of methanamine in MeOH (7.0 mL, 14.0 mmol), and Brettphos-Pd-G1 (23.5 mg, 0.0280 mmol). The mixture was stirred at 80° C. overnight. EtOAc was added and the mixture was washed with a saturated aqueous solution of NH₄Cl, water, brine, dried over Na₂SO₄, filtered, and concentrated under reduced pressure. The crude was purified by flash chromatography on silica gel using a gradient of EtOAc in n-heptane from 0% to 50%. Fractions were combined and concentrated under reduced pressure to afford 4-[[2-chloro-3-(methylamino)phenyl]-phenyl-methyl]piperidine-1-carboxylate as a beige foam (91.4 mg, 88% p., 64% y.). m/z [M+H]⁺=449.3, ¹H NMR (400 MHz, DMSO-d₆): δ (ppm) 7.38-7.29 (m, 7H), 7.26 (dd, J=8.3, 6.9 Hz, 2H), 7.17-7.11 (m, 2H), 6.88 (dd, J=7.8, 1.4 Hz, 1H), 6.43 (dd, J=8.1, 1.3 Hz, 1H), 5.43 (q, J=4.8 Hz, 1H), 5.05 (s, 2H), 4.09 (d, J=11.2 Hz, 1H), 3.95 (d, J=13.2 Hz, 2H), 2.82 (d, J=31.3 Hz, 3H), 2.70 (d, J=4.9 Hz, 3H), 2.47-2.38 (m, 1H), 1.53 (d, J=13.0 Hz, 1H), 1.36 (d, J=13.1 Hz, 1H), 1.07-0.91 (m, 2H).

### Intermediate 27B: 2-chloro-N-methyl-3-[phenyl(4-piperidyl)methyl]aniline

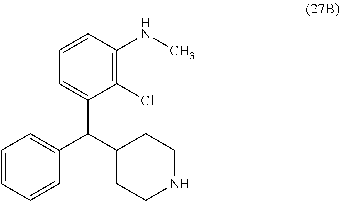 [SMILES: CNc1cccc(C(c2ccccc2)C2CCNCC2)c1Cl]

To a stirred solution of benzyl 4-[[2-chloro-3-(methylamino)phenyl]-phenyl-methyl]piperidine-1-carboxylate (91.4 mg, 0.179 mmol) in dry acetonitrile (1.8 mL) at ambient temperature under nitrogen was added iodo (trimethyl) silane (76.5 μL, 0.537 mmol) dropwise. The reaction mixture was stirred at ambient temperature for 1 h. The mixture was cooled to 0° C. and MeOH (87.1 μL, 2.15 mmol) was added dropwise. The mixture was stirred at ambient temperature for 1 h and was concentrated under reduced pressure. Et₂O was added and the resulting mixture was stirred at ambient temperature for 1 h. The solid was filtered, washed with Et₂O, and dried under reduced pressure. The solid was purified by reverse-phase preparative chromatography using a gradient of acetonitrile in water from 0% to 100% (0.1% AcOH). Acetonitrile was removed under removed pressure. The aqueous layer was basified with a saturated aqueous solution of NaHCO₃ was added. The aqueous layer was extracted with DCM (3×). The combined organic layers were washed with brine, dried over Na₂SO₄, filtered, and concentrated under reduced pressure, to afford 2-chloro-N-methyl-3-[phenyl(4-piperidyl)methyl]aniline as a white foam (29.0 mg, 92% p., 47% y.). m/z [M+H]⁺=315.3, ¹H NMR (400 MHz, DMSO-d₆): δ (ppm) 7.35-7.31 (m, 2H), 7.26 (dd, J=8.4, 6.9 Hz, 2H), 7.17-7.10 (m, 2H), 6.85 (dd, J=7.8, 1.4 Hz, 1H), 6.43 (dd, J=8.2, 1.4 Hz, 1H), 5.42 (q, J=4.8 Hz, 1H), 4.08 (d, J=11.2 Hz, 1H), 2.95 (dd, J=12.4, 3.3 Hz, 2H), 2.71 (d, J=4.9 Hz, 3H), 2.57-2.50 (m, 3H), 2.37-2.26 (m, 1H), 1.50 (d, J=12.8 Hz, 1H), 1.35 (d, J=13.1 Hz, 1H), 1.12-0.94 (m, 2H).

### Intermediate 27C: tert-butyl(4S)-5-amino-4-[5-[4-[[2-chloro-3-(methylamino)phenyl]-phenyl-methyl]piperidine-1-carbonyl]-6-methyl-1-oxo-isoindolin-2-yl]-5-oxo-pentanoate

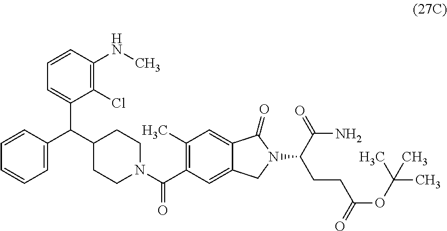 [SMILES: CNc1cccc(C(c2ccccc2)C2CCN(C(=O)c3cc4c(cc3C)C(=O)N([C@@H](CCC(=O)OC(C)(C)C)C(N)=O)C4)CC2)c1Cl]

To a stirred suspension of 2-chloro-N-methyl-3-[phenyl(4-piperidyl)methyl]aniline (28.0 mg, 0.0818 mmol) in EtOAc (820 μL) were added successively 2-[(1S)-4-tert-butoxy-1-carbamoyl-4-oxo-butyl]-6-methyl-1-oxo-isoindoline-5-carboxylic acid (36.0 mg, 0.0900 mmol), DIPEA (71.4 μL, 0.409 mmol), and a solution of T3P in EtOAc (50% w/w, 97.4 μL, 0.164 mmol). The reaction mixture was stirred at ambient temperature for 23 h. The reaction was quenched with the addition of a saturated aqueous solution of NaHCO₃. EtOAc was added to the mixture. The layers were separated and the aqueous layer was extracted with EtOAc (2×). The combined organic layers were washed with brine, dried using a phase separator cartridge, and concentrated under reduced pressure. The crude was purified by flash chromatography on silica gel using a gradient of EtOAc/EtOH (3/1) in n-heptane from 0% to 100%. Fractions were combined and concentrated under reduced pressure to afford tert-butyl(4S)-5-amino-4-[5-[4-[2-chloro-3-(methylamino)phenyl]-phenyl-methyl]piperidine-1-carbonyl]-6-methyl-1-oxo-isoindolin-2-yl]-5-oxo-pentanoate as a white solid (29.4 mg, 94% p., 50% y.). m/z [M-tBu+H]⁺=563.4, ¹H NMR (400 MHz, DMSO-d₆): δ (ppm) 7.56 (s, 2H), 7.47-7.07 (m, 8H), 6.89 (dd, J=25.1, 7.8 Hz, 1H), 6.48-6.39 (m, 1H), 5.43 (dd, J=18.4, 5.1 Hz, 1H), 4.75-4.69 (m, 1H), 4.59-4.48 (m, 2H), 4.41 (d, J=17.3 Hz, 1H), 4.13 (t, J=10.0 Hz, 1H), 3.21 (s, 1H), 2.97 (q, J=12.8 Hz, 1H), 2.76 (s, 1H), 2.70 (dd, J=16.3, 4.9 Hz, 3H), 2.33 (d, J=5.6 Hz, 1H), 2.23 (s, 2H), 2.14 (s, 3H), 1.96 (s, 1H), 1.67 (s, 1H), 1.47 (s, 1H), 1.35-1.26 (m, 9H), 1.08 (d, J=19.3 Hz, 3H).

### Example 27

In a sealed vial, to a solution of tert-butyl(4S)-5-amino-4-[5-[4-[[2-chloro-3-(methylamino)phenyl]-phenyl-methyl]piperidine-1-carbonyl]-6-methyl-1-oxo-isoindolin-2-yl]-5-oxo-pentanoate (27.7 mg, 0.0386 mmol) in acetonitrile (230 μL) was added 4-methylbenzenesulfonic acid hydrate (22.0 mg, 0.12 mmol). The solution was stirred at 90° C. for 1 h. The mixture was diluted with EtOAc and washed with a saturated aqueous solution of NaHCO₃ and brine. The organic layer was dried over Na₂SO₄, filtered, and concentrated under reduced pressure. The crude was purified by reverse-phase preparative chromatography using a gradient of acetonitrile in water from 0% to 100% (0.1% AcOH). The desired fractions were combined and concentrated under reduced pressure until acetonitrile was evaporated. A saturated aqueous solution of NaHCO₃ was added. The resulting suspension was filtered and dried under at 50° C. under reduced pressure overnight to afford ((3S)-3-[5-[4-[[2-chloro-3-(methylamino)phenyl]-phenyl-methyl]piperidine-1-carbonyl]-6-methyl-1-oxo-isoindolin-2-yl]piperidine-2,6-dione as a white powder (10.8 mg, 99.6% p., 46% y.). m/z [M+H]⁺=599.4, ¹H NMR (500 MHz, DMSO-d₆): δ (ppm) 10.97 (br s, 1H), 7.66-7.56 (m, 1H), 7.51-7.37 (m, 1H), 7.33 (br d, J=7.3 Hz, 2H), 7.30-7.20 (m, 2H), 7.19-7.14 (m, 1H), 7.14-7.06 (m, 1H), 6.99-6.80 (m, 1H), 6.46 (dd, J=8.1, 1.1 Hz, 1H), 5.50-5.33 (m, 1H), 5.18-5.02 (m, 1H), 4.60-4.47 (m, 1H), 4.47-4.35 (m, 1H), 4.35-4.21 (m, 1H), 4.13 (s, 1H), 3.28-3.13 (m, 1H), 3.07-2.94 (m, 1H), 2.94-2.84 (m, 1H), 2.83-2.74 (m, 1H), 2.73-2.63 (m, 3H), 2.62-2.54 (m, 1H), 2.54-2.52 (m, 1H), 2.48-2.35 (m, 1H), 2.35-2.19 (m, 3H), 2.10-1.86 (m, 1H), 1.74-1.24 (m, 2H), 1.23-0.76 (m, 2H).

### Example 28

### (3S)-3-[5-[4-[[2-chloro-3-(dimethylamino)phenyl]-phenyl-methyl]piperidine-1-carbonyl]-6-methyl-1-oxo-isoindolin-2-yl]piperidine-2,6-dione

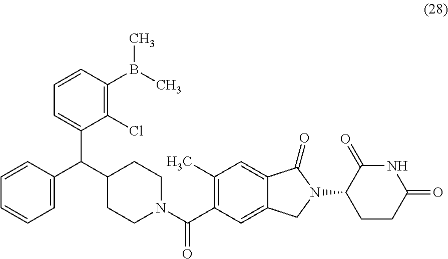 [SMILES: CB(C)c1cccc(C(c2ccccc2)C2CCN(C(=O)c3cc4c(cc3C)C(=O)N([C@H]3CCC(=O)NC3=O)C4)CC2)c1Cl]

### Intermediate 28A: benzyl 4-[[2-chloro-3-(dimethylamino)phenyl]-phenyl-methyl]piperidine-1-carboxylate

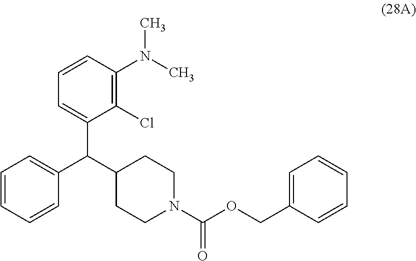 [SMILES: CN(C)c1cccc(C(c2ccccc2)C2CCN(C(=O)OCc3ccccc3)CC2)c1Cl]

In a sealed vial, to a stirred solution of benzyl 4-[(3-bromo-2-chloro-phenyl)-phenyl-methyl]piperidine-1-carboxylate (150 mg, 0.280 mmol) in tert-butanol (1.1 mL) under argon were added successively t-BuONa (32.3 mg, 0.336 mmol), a 2 M solution of N-methylmethanamine in MeOH (7.0 mL, 14.0 mmol), and Brettphos-Pd-G1 (23.5 mg, 0.0280 mmol). The mixture was stirred at 80° C. overnight. Additional t-BuONa (32.3 mg, 0.336 mmol), a 2 M solution of N-methylmethanamine in MeOH (4.2 mL, 8.39 mmol), and Brettphos-Pd-G1 (23.5 mg, 0.0280 mmol) were added and the reaction mixture was stirred at 80° C. for 23 h. EtOAc was added and the mixture was washed with a saturated aqueous solution of NH₄Cl, water, brine, dried over Na₂SO₄, filtered, and concentrated under reduced pressure. The crude was purified by flash chromatography on silica gel using a gradient of EtOAc in n-heptane from 0% to 50%. Fractions were combined and concentrated under reduced pressure to afford benzyl 4-[[2-chloro-3-(dimethylamino)phenyl]-phenyl-methyl]piperidine-1-carboxylate as a beige foam (89.1 mg, 80% p., 55% y.). m/z [M+H]⁺=463.3, ¹H NMR (400 MHz, DMSO-d₆): δ (ppm) 7.39-7.29 (m, 8H), 7.27 (td, J=7.7, 2.1 Hz, 3H), 7.18-7.13 (m, 1H), 7.00 (dd, J=7.9, 1.5 Hz, 1H), 5.05 (s, 2H), 4.21 (d, J=11.2 Hz, 1H), 4.00-3.90 (m, 2H), 2.91-2.69 (m, 2H), 2.64 (s, 6H), 2.49 (s, 1H), 1.46 (d, J=13.3 Hz, 1H), 1.37 (d, J=13.2 Hz, 1H), 1.08-0.92 (m, 2H).

### Intermediate 28B: 2-chloro-N,N-dimethyl-3-[phenyl(4-piperidyl)methyl]aniline hydroiodide

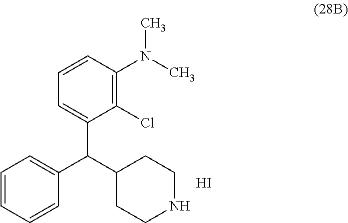

To a stirred solution of benzyl 4-[2-chloro-3-(dimethylamino)phenyl]-phenyl-methyl]piperidine-1-carboxylate (89.1 mg, 0.154 mmol) in dry acetonitrile (1.5 mL) at ambient temperature under nitrogen was added iodo (trimethyl) silane (65.7 μL, 0.462 mmol) dropwise. The reaction mixture was stirred at ambient temperature for 1 h. The mixture was cooled to 0° C. and MeOH (74.8 μL, 1.85 mmol) was added dropwise. The mixture was stirred at ambient temperature for 1 h and was concentrated under reduced pressure. Et₂O was added and the resulting mixture was stirred at ambient temperature overnight. The solid was filtered, washed with Et₂O, and dried under reduced pressure overnight to afford 2-chloro-N,N-dimethyl-3-[phenyl(4-piperidyl)methyl]aniline hydroiodide as a golden solid (90 mg, 77% p., 98% y.). m/z [M+H]⁺=329.3, ¹H NMR (400 MHz, DMSO-d₆): δ (ppm) 8.40 (s, 1H), 8.08 (s, 1H), 7.40-7.35 (m, 2H), 7.31 (tdd, J=7.7, 5.2, 2.3 Hz, 4H), 7.22-7.16 (m, 1H), 7.08 (dd, J=7.5, 1.9 Hz, 1H), 4.21 (d, J=11.2 Hz, 1H), 3.23 (d, J=10.9 Hz, 2H), 2.83 (t, J=10.7 Hz, 2H), 2.69 (s, 6H), 2.57 (d, J=11.6 Hz, 1H), 1.54 (dd, J=24.3, 14.2 Hz, 2H), 1.33-1.16 (m, 2H).

### Intermediate 28C: tert-butyl(4S)-5-amino-4-[5-[4-[[2-chloro-3-(dimethylamino)phenyl]-phenyl-methyl]piperidine-1-carbonyl]-6-methyl-1-oxo-isoindolin-2-yl]-5-oxo-pentanoate

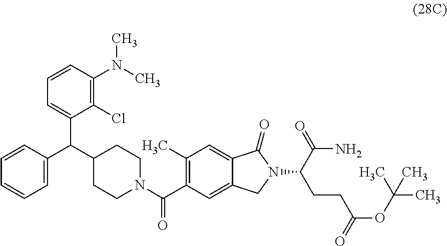 [SMILES: Cc1cc2c(cc1C(=O)N1CCC(C(c3ccccc3)c3cccc(N(C)C)c3Cl)CC1)CN([C@@H](CCC(=O)OC(C)(C)C)C(N)=O)C2=O]

To a stirred suspension of 2-chloro-N,N-dimethyl-3-[phenyl(4-piperidyl)methyl]aniline hydroiodide (87.1 mg, 0.147 mmol) in EtOAc (1.5 mL) were added successively 2-[(1S)-4-tert-butoxy-1-carbamoyl-4-oxo-butyl]-6-methyl-1-oxo-isoindoline-5-carboxylic acid (64.7 mg, 0.162 mmol), DIPEA (128 μL, 0.734 mmol), and a solution of T3P in EtOAc (50% w/w, 175 μL, 0.294 mmol). The reaction mixture was stirred at ambient temperature for 23 h. Additional DIPEA (128 μL, 0.734 mmol) and a solution of T3P in EtOAc (50% w/w, 175 μL, 0.294 mmol) were added the reaction mixture was stirred at 60° C. for 1 h. The reaction was quenched with the addition of a saturated aqueous solution of NaHCO₃. EtOAc was added to the mixture. The layers were separated and the aqueous layer was extracted with EtOAc (2×). The combined organic layers were washed with brine, dried using a phase separator cartridge, and concentrated under reduced pressure. The crude was purified by flash chromatography on silica gel using a gradient of EtOAc/EtOH (3/1) (0.5% conc. NH₄OH) in n-heptane from 0% to 100%. Fractions were combined and concentrated under reduced pressure to afford tert-butyl(4S)-5-amino-4-[5-[4-[[2-chloro-3-(dimethylamino)phenyl]-phenyl-methyl]piperidine-1-carbonyl]-6-methyl-1-oxo-isoindolin-2-yl]-5-oxo-pentanoate as an off-white foam (55.0 mg, 89% p., 49% y.). m/z [M+H]⁺=687.5, ¹H NMR (400 MHz, DMSO-d₆): δ (ppm) 7.56 (s, 2H), 7.48-7.10 (m, 10H), 7.05-6.96 (m, 1H), 4.75-4.67 (m, 1H), 4.54 (d, J=17.5 Hz, 2H), 4.46-4.35 (m, 1H), 4.25 (t, J=11.1 Hz, 1H), 3.24 (d, J=19.7 Hz, 1H), 2.95 (t, J=12.4 Hz, 1H), 2.77 (d, J=11.5 Hz, 1H), 2.66 (s, 3H), 2.62 (d, J=1.2 Hz, 3H), 2.55 (s, 1H), 2.33 (d, J=4.8 Hz, 1H), 2.23 (s, 2H), 2.14 (s, 3H), 1.97 (s, 1H), 1.55 (d, J=44.0 Hz, 1H), 1.35-1.26 (m, 10H), 1.10 (s, 1H).

### Example 28

In a sealed vial, to a solution of tert-butyl(4S)-5-amino-4-[5-[4-[[2-chloro-3-(dimethylamino)phenyl]-phenyl-methyl]piperidine-1-carbonyl]-6-methyl-1-oxo-isoindolin-2-yl]-5-oxo-pentanoate (61.8 mg, 0.0800 mmol) in acetonitrile (477 μL) was added 4-methylbenzenesulfonic acid hydrate (45.7 mg, 0.240 mmol). The solution was stirred at 90° C. for 1 h. The mixture was diluted with EtOAc and washed with a saturated aqueous solution of NaHCO₃ and brine. The organic layer was dried over Na₂SO₄, filtered, and concentrated under reduced pressure. The crude was purified by reverse-phase preparative chromatography using a gradient of acetonitrile in water from 0% to 100% (0.1% AcOH). The desired fractions were combined and concentrated under reduced pressure until acetonitrile was evaporated. A saturated aqueous solution of NaHCO₃ was added. The resulting suspension was filtered and dried under at 50° C. under reduced pressure overnight to afford (3S)-3-[5-[4-[2-chloro-3-(dimethylamino)phenyl]-phenyl-methyl]piperidine-1-carbonyl]-6-methyl-1-oxo-isoindolin-2-yl]piperidine-2,6-dione as a white powder (14.1 mg, 93.6% p., 27% y.). m/z [M+H]⁺=613.3, ¹H NMR (500 MHz, DMSO-d₆): δ (ppm) 10.97 (s, 1H), 7.67-7.56 (m, 1H), 7.51-7.39 (m, 1H), 7.39-7.34 (m, 2H), 7.34-7.32 (m, 1H), 7.29 (s, 1H), 7.29-7.20 (m, 2H), 7.20-7.09 (m, 1H), 7.07-6.93 (m, 1H), 5.21-4.98 (m, 1H), 4.60-4.47 (m, 1H), 4.47-4.36 (m, 1H), 4.35-4.20 (m, 2H), 3.28-3.16 (m, 1H), 3.06-2.93 (m, 1H), 2.93-2.83 (m, 1H), 2.82-2.72 (m, 1H), 2.71-2.61 (m, 6H), 2.60-2.52 (m, 2H), 2.47-2.35 (m, 1H), 2.25 (s, 3H), 2.09-1.86 (m, 1H), 1.69-1.25 (m, 2H), 1.23-0.75 (m, 2H).

### Example 29

### (3S)-3-[5-[4-[(R)-(2-chloro-3-hydroxy-phenyl)-phenyl-methyl]piperidine-1-carbonyl]-6-methyl-1-oxo-isoindolin-2-yl]piperidine-2,6-dione

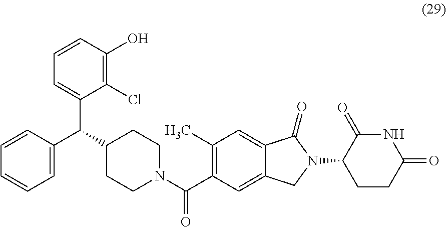 [SMILES: Cc1cc2c(cc1C(=O)N1CCC([C@H](c3ccccc3)c3cccc(O)c3Cl)CC1)CN([C@H]1CCC(=O)NC1=O)C2=O]

### Intermediate 29A: tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate

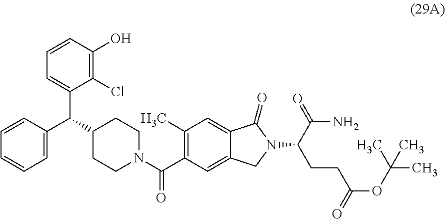 [SMILES: Cc1cc2c(cc1C(=O)N1CCC([C@H](c3ccccc3)c3cccc(O)c3Cl)CC1)CN([C@@H](CCC(=O)OC(C)(C)C)C(N)=O)C2=O]

To a stirred suspension of 2-chloro-3-[(R)-phenyl(4-piperidyl)methyl]phenol hydroiodide (91.8 mg, 0.214 mmol) in EtOAc (2.1 mL) were added successively 2-[(1S)-4-tert-butoxy-1-carbamoyl-4-oxo-butyl]-6-methyl-1-oxo-isoindoline-5-carboxylic acid (93.1 mg, 0.235 mmol), DIPEA (187 μL, 1.07 mmol), and a solution of T3P in EtOAc (50% w/w, 254 μL, 0.427 mmol). The reaction mixture was stirred at ambient temperature for 5 h. The reaction was quenched with the addition of a saturated aqueous solution of NaHCO₃. EtOAc was added to the mixture. The layers were separated and the aqueous layer was extracted with EtOAc (2×). The combined organic layers were washed with brine, dried using a phase separator cartridge, and concentrated under reduced pressure. The crude was purified by reverse-phase preparative chromatography using a gradient of acetonitrile in water from 0% to 100% (0.1% AcOH). Acetonitrile was evaporated and saturated aqueous NaHCO₃ was added. The aqueous layer was extracted with DCM (3×). The combined organic layers were washed with brine, dried over Na₂SO₄, filtered, and concentrated under reduced pressure to afford tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate as a white foam (64.6 mg, 98% p.45% y.). m/z [M+H]⁺=660.5, ¹H NMR (400 MHz, DMSO-d₆): δ (ppm) 10.04 (s, 1H), 7.55 (d, J=7.8 Hz, 2H), 7.44 (d, J=15.2 Hz, 1H), 7.39-7.06 (m, 10H), 6.77 (dt, J=23.7, 4.7 Hz, 1H), 4.74-4.68 (m, 1H), 4.54 (d, J=18.7 Hz, 2H), 4.42 (d, J=16.8 Hz, 1H), 3.22 (bs, 2H), 2.98 (t, J=12.8 Hz, 1H), 2.76 (s, 1H), 2.33 (d, J=4.6 Hz, 1H), 2.23 (s, 2H), 2.14 (s, 3H), 1.95 (d, J=9.2 Hz, 1H), 1.56 (d, J=69.4 Hz, 2H), 1.34-1.26 (m, 9H), 1.11 (s, 1H), [α]_{D}=+5.0°·mL·dm⁻¹.g⁻¹[20° C., Na lamp, 589 nm, C=2.2 mg/mL, EtOH].

### Example 29

To a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (63.3 mg, 0.0959 mmol) in acetonitrile (650 μL) at ambient temperature was added 4-methylbenzenesulfonic acid hydrate (54.7 mg, 0.288 mmol). The reaction mixture was stirred at 80° C. for 2 h. The reaction mixture was cooled to ambient temperature and diluted with water. The aqueous layer was extracted with EtOAc (2×). The combined organic layers were washed with brine, dried over Na₂SO₄, filtered, and concentrated under reduced pressure. The crude was purified by flash chromatography on silica gel using a gradient of EtOAc/EtOH (3/1) in cyclohexane from 10% to 100%. The residue was solubilized in a minimum amount of DCM and the homogenous solution was added to a stirred solution of n-pentane dropwise. The resulting precipitate was triturated for 1 h, filtered, washed with n-pentane, and dried under reduced pressure at 50° C. overnight to afford (3S)-3-[5-[4-[(R)-(2-chloro-3-hydroxy-phenyl)-phenyl-methyl]piperidine-1-carbonyl]-6-methyl-1-oxo-isoindolin-2-yl]piperidine-2,6-dione as a white powder (18.9 mg, 98.8% p. 33% y.). m/z [M+H]⁺=586.3, ¹H NMR (500 MHz, DMSO-d₆): δ (ppm) 10.98 (s, 1H), 10.05-9.92 (m, 1H), 7.62-7.59 (m, 1H), 7.51-7.26 (m, 4H), 7.24 (br t, J=7.7 Hz, 1H), 7.20-7.11 (m, 2H), 7.11-7.05 (m, 1H), 6.84-6.72 (m, 1H), 5.13-5.07 (m, 1H), 4.52 (br s, 1H), 4.48-4.24 (m, 2H), 4.20-4.14 (m, 1H), 3.29-3.16 (m, 1H), 3.06-2.69 (m, 3H), 2.60 (br d, J=17.6 Hz, 2H), 2.42-2.18 (m, 4H), 1.99 (br s, 1H), 1.73-0.84 (m, 4H).

### Example 30

### (3S)-3-[5-[4-[(2-chloro-3-oxazol-2-yl-phenyl)-phenyl-methyl]piperidine-1-carbonyl]-6-methyl-1-oxo-isoindolin-2-yl]piperidine-2,6-dione

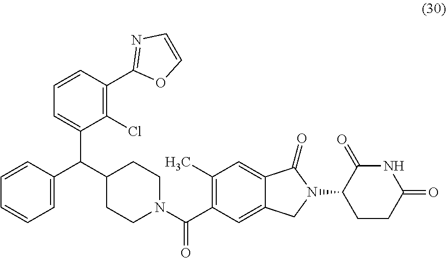 [SMILES: Cc1cc2c(cc1C(=O)N1CCC(C(c3ccccc3)c3cccc(-c4ncco4)c3Cl)CC1)CN([C@H]1CCC(=O)NC1=O)C2=O]

### Intermediate 30A: benzyl 4-[(2-chloro-3-oxazol-2-yl-phenyl)-phenyl-methyl]piperidine-1-carboxylate

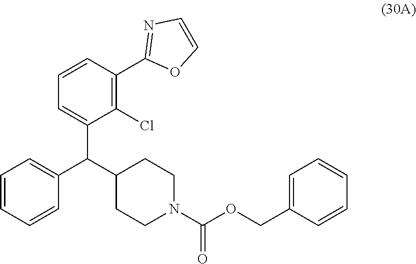 [SMILES: O=C(OCc1ccccc1)N1CCC(C(c2ccccc2)c2cccc(-c3ncco3)c2Cl)CC1]

A sealed vial was successively charged with benzyl 4-[[2-chloro-3-(4,4,5,5-tetramethyl-1,3,2-dioxaborolan-2-yl)phenyl]-phenyl-methyl]piperidine-1-carboxylate (200 mg, 0.293 mmol), 2-bromo-1,3-oxazole (43.4 mg, 0.293 mmol), and Cs₂CO₃ (292 mg, 0.879 mmol) in a mixture of 1,4-dioxane (4.0 mL) and water (0.5 mL). The reaction mixture was degassed with nitrogen for 10 min. Pd(PPh₃)₄ (16.9 mg, 0.0147 mmol) was added and the reaction mixture was stirred at 90° C. for 1 h. The reaction mixture was diluted with water and EtOAc. The layers were separated, and the aqueous layer was extracted with EtOAc (2×). The combined organic layers were washed with brine, dried over hydrophobic paper, and concentrated under reduced pressure to afford benzyl 4-[(2-chloro-3-oxazol-2-yl-phenyl)-phenyl-methyl]piperidine-1-carboxylate as a brown gum (218 mg, 72% p., quant. yield)). m/z [M+H]⁺=487.3.

### Intermediate 30B: 2-[2-chloro-3-[phenyl(4-piperidyl)methyl]phenyl]oxazole hydroiodide

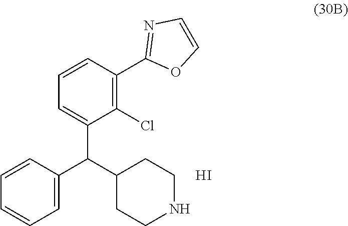

To a stirred solution of benzyl 4-[(2-chloro-3-oxazol-2-yl-phenyl)-phenyl-methyl]piperidine-1-carboxylate (218 mg, 0.323 mmol) in dry acetonitrile (3.2 mL) at ambient temperature under nitrogen was added iodo (trimethyl) silane (138 μL, 0.969 mmol) dropwise. The reaction mixture was stirred at ambient temperature for 1 h. The mixture was cooled to 0° C. and MeOH (157 μL, 3.87 mmol) was added dropwise. The mixture was stirred at ambient temperature for 1 h and was concentrated under reduced pressure. Et₂O was added and the resulting mixture was stirred at ambient temperature for 1 h. The solid was filtered, washed with Et₂O, and dried under reduced pressure to afford 2-[2-chloro-3-[phenyl(4-piperidyl)methyl]phenyl]oxazole hydroiodide as a brown solid (222 mg, 61% p., 87% y.). m/z [M+H]⁺=353.3.

### Intermediate 30C: tert-butyl(4S)-5-amino-4-[5-[4-[(2-chloro-3-oxazol-2-yl-phenyl)-phenyl-methyl]piperidine-1-carbonyl]-6-methyl-1-oxo-isoindolin-2-yl]-5-oxo-pentanoate

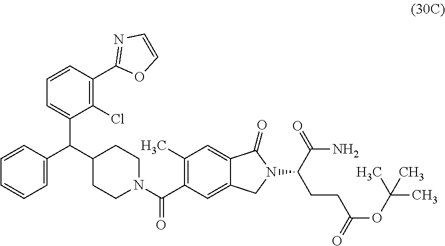 [SMILES: Cc1cc2c(cc1C(=O)N1CCC(C(c3ccccc3)c3cccc(-c4ncco4)c3Cl)CC1)CN([C@@H](CCC(=O)OC(C)(C)C)C(N)=O)C2=O]

To a stirred solution of 2-[(1S)-4-tert-butoxy-1-carbamoyl-4-oxo-butyl]-6-methyl-1-oxo-isoindoline-5-carboxylic acid (37.8 mg, 0.0954 mmol), a 0.5 M solution of HATU in DMF (286 μL, 0.143 mmol), and DIPEA (83.3 μL, 0.477 mmol) in DMF (1.0 mL), 2-[2-chloro-3-[phenyl(4-piperidyl)methyl]phenyl]oxazole hydroiodide (75.2 mg, 0.0954 mmol) were added. The reaction mixture was stirred at ambient temperature overnight. The reaction was quenched with water. EtOAc and FastWorX® powder were added to the mixture. The organic solvent was evaporated under reduced pressure. The resin was filtered through a dry-loading cartridge and washed with water, an aqueous saturated aqueous solution of NaHCO₃, and water. The cartridge was flushed with nitrogen for 5 min and used for chromatography. The crude was purified by flash chromatography on silica gel using a gradient of EtOAc/EtOH (3/1) in n-heptane from 0% to 100% to afford tert-butyl(4S)-5-amino-4-[5-[4-[(2-chloro-3-oxazol-2-yl-phenyl)-phenyl-methyl]piperidine-1-carbonyl]-6-methyl-1-oxo-isoindolin-2-yl]-5-oxo-pentanoate as an orange solid (33.0 mg, 91% p., 44% y.). m/z [M+H]⁺=711.3.

### Example 30

To a stirred solution of tert-butyl(4S)-5-amino-4-[5-[4-[(2-chloro-3-oxazol-2-yl-phenyl)-phenyl-methyl]piperidine-1-carbonyl]-6-methyl-1-oxo-isoindolin-2-yl]-5-oxo-pentanoate (46.0 mg, 0.0589 mmol) in acetonitrile (500 μL) at ambient temperature was added 4-methylbenzenesulfonic acid hydrate (33.6 mg, 0.177 mmol). The reaction mixture was stirred at 80° C. for 2 h. The reaction mixture was cooled to ambient temperature and diluted with water. The aqueous layer was extracted with EtOAc (2×).

The combined organic layers were washed with brine, dried over Na₂SO₄, filtered, and concentrated under reduced pressure. The crude was purified by flash chromatography on silica gel using a gradient of EtOAc/EtOH (3/1) in cyclohexane from 10% to 100%. The residue was solubilized in a minimum amount of DCM and the homogenous solution was added to n-pentane dropwise. The resulting precipitate was triturated for 1 h, filtered, washed with n-pentane, and dried under reduced pressure at 50° C. overnight to afford (3S)-3-[5-[4-[(2-chloro-3-oxazol-2-yl-phenyl)-phenyl-methyl]piperidine-1-carbonyl]-6-methyl-1-oxo-isoindolin-2-yl]piperidine-2,6-dione as a white powder (11.0 mg, 97.8% p. 29% y.). m/z [M+H]⁺=637.3, ¹H NMR (500 MHz, DMSO-d₆): δ (ppm) 11.04-10.89 (m, 1H), 8.39-8.18 (m, 1H), 7.99-7.87 (m, 1H), 7.73-7.63 (m, 1H), 7.62-7.35 (m, 6H), 7.35-7.24 (m, 2H), 7.23-7.12 (m, 1H), 5.14-5.06 (m, 1H), 4.62-4.19 (m, 4H), 3.27 (s, 1H), 3.05-2.57 (m, 5H), 2.45-2.22 (m, 4H), 2.06-1.93 (m, 1H), 1.71-1.05 (m, 4H).

### Example 31

### (3S)-3-[5-[4-[(R)-[2-chloro-3-(2-hydroxyethoxy)phenyl]-phenyl-methyl]piperidine-1-carbonyl]-6-methyl-1-oxo-isoindolin-2-yl]piperidine-2,6-dione

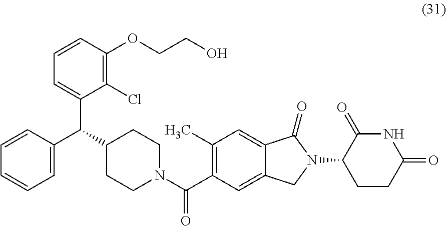 [SMILES: Cc1cc2c(cc1C(=O)N1CCC([C@H](c3ccccc3)c3cccc(OCCO)c3Cl)CC1)CN([C@H]1CCC(=O)NC1=O)C2=O]

### Intermediate 31A: benzyl 4-[(R)-[3-[2-[tert-butyl(dimethyl) silyl]oxyethoxy]-2-chloro-phenyl]-phenyl-methyl]piperidine-1-carboxylate

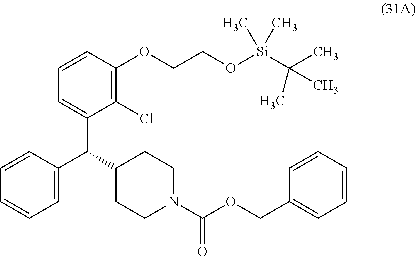 [SMILES: CC(C)(C)[Si](C)(C)OCCOc1cccc([C@@H](c2ccccc2)C2CCN(C(=O)OCc3ccccc3)CC2)c1Cl]

In a sealed vial, to a stirred solution of benzyl 4-[(R)-(2-chloro-3-hydroxy-phenyl)-phenyl-methyl]piperidine-1-carboxylate (250 mg, 0.573 mmol) in THF (2.9 mL) were added successively 2-[tert-butyl(dimethyl) silyl]oxyethanol (170 μL, 0.860 mmol), triphenylphosphane (226 mg, 0.860 mmol), and then DIAD (169 μL, 0.860 mmol). The reaction mixture was stirred at ambient temperature overnight. The reaction mixture was concentrated under reduced pressure. The crude was purified by flash chromatography on silica gel using a gradient of EtOAc in cyclohexane from 1% to 30%. The desired fractions were combined and concentrated under reduced pressure to afford benzyl 4-[(R)-[3-[2-[tert-butyl(dimethyl) silyl]oxyethoxy]-2-chloro-phenyl]-phenyl-methyl]piperidine-1-carboxylate as a colorless oil (297 mg, 99% p., 86% y.). m/z [M+H]⁺=594.4, ¹H NMR (400 MHz, DMSO-d₆): δ (ppm) 7.38-7.29 (m, 7H), 7.29-7.23 (m, 4H), 7.20-7.11 (m, 1H), 6.99-6.90 (m, 1H), 5.05 (s, 2H), 4.17 (d, J=11.2 Hz, 1H), 4.05 (ddt, J=15.2, 10.5, 5.0 Hz, 2H), 3.99-3.87 (m, 4H), 2.78 (s, 3H), 1.46 (d, J=13.2 Hz, 1H), 1.38 (d, J=12.6 Hz, 1H), 1.10-0.89 (m, 2H), 0.84 (s, 9H), 0.04 (d, J=2.4 Hz, 6H), [α]_{D}=+55°·mL·dm⁻¹.g⁻¹[20° C., Na lamp, 589 nm, C=1.0 mg/mL, MeOH].

### Intermediate 31B: 2-[2-chloro-3-[(R)-phenyl(4-piperidyl)methyl]phenoxy]ethanol hydroiodide

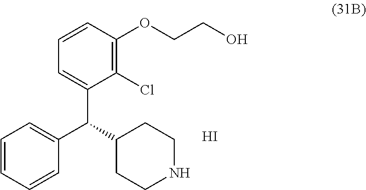

In a round-bottom flask, to a stirred solution of benzyl 4-[(R)-[3-[2-[tert-butyl (dimethyl) silyl]oxyethoxy]-2-chloro-phenyl]-phenyl-methyl]piperidine-1-carboxylate (294 mg, 0.490 mmol) in dry acetonitrile (4.9 mL) at ambient temperature under nitrogen was added iodo (trimethyl) silane (209 μL, 1.47 mmol) dropwise. The reaction mixture was stirred at ambient temperature for 3 h. The mixture was cooled to 0° C. and MeOH (238 μL, 5.88 mmol) was added dropwise. The mixture was stirred at ambient temperature for 1 h and was concentrated under reduced pressure. Et₂O was added and the resulting mixture was stirred at ambient temperature for 1 h. The solid was filtered, washed with Et₂O, and dried under reduced pressure at 50° C. to afford (2-[2-chloro-3-[(R)-phenyl(4-piperidyl)methyl]phenoxy]ethanol hydroiodide as a pale brown solid (230 mg, 92% p., 91% y.). m/z [M+H]⁺=346.3, ¹H NMR (400 MHz, DMSO-d₆): δ (ppm) 8.41 (s, 1H), 8.08 (s, 1H), 7.39-7.34 (m, 2H), 7.29 (td, J=7.7, 4.1 Hz, 3H), 7.20 (dq, J=7.9, 1.8 Hz, 2H), 6.98 (dd, J=8.2, 1.4 Hz, 1H), 4.83 (t, J=5.4 Hz, 1H), 4.17 (d, J=11.2 Hz, 1H), 4.01 (hept, J=5.1 Hz, 2H), 3.71 (q, J=5.0 Hz, 2H), 3.24 (d, J=7.6 Hz, 2H), 2.83 (s, 2H), 2.57 (d, J=11.4 Hz, 1H), 1.56 (t, J=16.4 Hz, 2H), 1.26 (p, J=12.4 Hz, 2H), [α]_{D}=+55°·mL·dm 1.g⁻¹[20° C., Na lamp, 589 nm, C=1.3 mg/mL, MeOH].

### Intermediate 31C: tert-butyl(4S)-5-amino-4-[5-[4-[(R)-[2-chloro-3-(2-hydroxyethoxy)phenyl]-phenyl-methyl]piperidine-1-carbonyl]-6-methyl-1-oxo-isoindolin-2-yl]-5-oxo-pentanoate

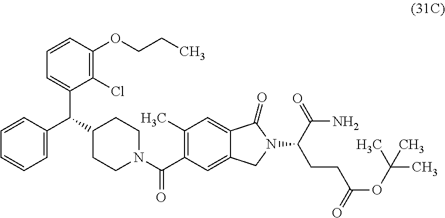 [SMILES: CCCOc1cccc([C@@H](c2ccccc2)C2CCN(C(=O)c3cc4c(cc3C)C(=O)N([C@@H](CCC(=O)OC(C)(C)C)C(N)=O)C4)CC2)c1Cl]

In a round-bottom flask, to a stirred suspension of 2-[(1S)-4-tert-butoxy-1-carbamoyl-4-oxo-butyl]-6-methyl-1-oxo-isoindoline-5-carboxylic acid (175 mg, 0.465 mmol) in DMF (2.2 mL) were added successively 2-[2-chloro-3-[(R)-phenyl(4-piperidyl)methyl]phenoxy]ethanol hydroiodide (251 mg, 0.488 mmol), DIPEA (410 μL, 2.32 mmol) then a solution of 0.5 M HATU in DMF (1.4 mL, 0.697 mmol). The reaction mixture was stirred at ambient temperature for 1 h. The reaction was quenched with the addition of water. The mixture was basified with a saturated aqueous solution of NaHCO₃. EtOAc and then FastWorX® powder were added. The organic layer was concentrated under reduced pressure. The reaction mixture was filtered through a dry-loading cartridge which was rinsed with water. The crude was purified by flash chromatography on silica gel using a gradient of EtOAc/EtOH (3/1+0.5% NH₃) in cyclohexane from 10% to 100% to afford tert-butyl(4S)-5-amino-4-[5-[4-[(R)-[2-chloro-3-(2-hydroxyethoxy)phenyl]-phenyl-methyl]piperidine-1-carbonyl]-6-methyl-1-oxo-isoindolin-2-yl]-5-oxo-pentanoate as an off-white solid (194 mg, 89% p., 53% y.). m/z [M+H]⁺=704.3. [α]_{D}=+16°·mL·dm⁻¹ g⁻¹[20° C., Na lamp, 589 nm, C=1.4 mg/mL, MeOH].

### Example 31

To a stirred solution of tert-butyl(4S)-5-amino-4-[5-[4-[(R)-[2-chloro-3-(2-hydroxyethoxy)phenyl]-phenyl-methyl]piperidine-1-carbonyl]-6-methyl-1-oxo-isoindolin-2-yl]-5-oxo-pentanoate (211 mg, 0.267 mmol) in acetonitrile (2.7 mL) was added 4-methylbenzenesulfonic acid hydrate (254 mg, 1.33 mmol). The reaction mixture was stirred at 80° C. for 5 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by flash chromatography on silica gel using a gradient of EtOAc/EtOH (3/1) in cyclohexane from 10% to 100% and by reverse-phase preparative chromatography using a gradient of acetonitrile in water from 0% to 100% (0.1% AcOH). Acetonitrile was removed under reduced pressure. The aqueous layer was basified with a saturated aqueous solution of NaHCO₃. The precipitate was triturated for 1 h, filtered, washed with water, and dried under reduced pressure at 50° C. overnight to afford ((3S)-3-[5-[4-[(R)-[2-chloro-3-(2-hydroxyethoxy)phenyl]-phenyl-methyl]piperidine-1-carbonyl]-6-methyl-1-oxo-isoindolin-2-yl]piperidine-2,6-dione as a white solid (70.4 mg, 99.8% p., 42% y.). m/z [M+H]⁺=630.3, ¹H NMR (600 MHz, DMSO-d₆): δ (ppm) 11.05-10.88 (m, 1H), 7.66-7.55 (m, 1H), 7.52-7.39 (m, 1H), 7.38-7.31 (m, 2H), 7.31-7.20 (m, 4H), 7.20-7.10 (m, 1H), 7.01-6.87 (m, 1H), 5.15-5.05 (m, 1H), 4.81 (br d, J=19.1 Hz, 1H), 4.58-4.47 (m, 1H), 4.46-4.35 (m, 1H), 4.35-4.27 (m, 1H), 4.26-4.16 (m, 1H), 4.08-3.93 (m, 2H), 3.77-3.66 (m, 2H), 3.28-3.14 (m, 1H), 3.05-2.94 (m, 1H), 2.94-2.84 (m, 1H), 2.84-2.69 (m, 1H), 2.65-2.53 (m, 2H), 2.44-2.35 (m, 1H), 2.35-2.18 (m, 3H), 1.99 (s, 1H), 1.70-1.25 (m, 2H), 1.17 (s, 2H), [α]_{D}=+10°·mL·dm 1 g⁻¹[20° C., Na lamp, 589 nm, C=1.3 mg/mL, DMSO].

### Example 32

### (3S)-3-[5-[4-[(2-chlorophenyl)-isothiazol-5-yl-methyl]piperidine-1-carbonyl]-6-methyl-1-oxo-isoindolin-2-yl]piperidine-2,6-dione

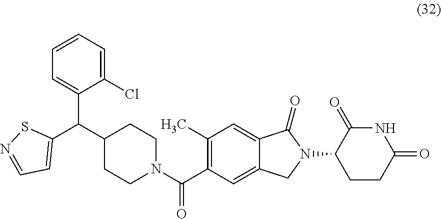 [SMILES: Cc1cc2c(cc1C(=O)N1CCC(C(c3ccns3)c3ccccc3Cl)CC1)CN([C@H]1CCC(=O)NC1=O)C2=O]

### Intermediate 32A: tert-butyl 4-[methoxy(methyl) carbamoyl]piperidine-1-carboxylate

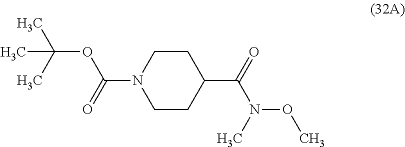 [SMILES: CON(C)C(=O)C1CCN(C(=O)OC(C)(C)C)CC1]

In a three neck round-bottom flask equipped with a thermometer, a solution of 1-(tert-butoxycarbonyl) piperidine-4-carboxylic acid (14.8 g, 64.6 mmol) in dry DCM (150 mL) was cooled to 0° C. under argon. Di(imidazol-1-yl)methanone (11.5 g, 71.0 mmol) was added and the mixture was stirred at 0° C. for 30 min. Then, triethylamine (12 mL, 87.1 mmol) and N-methoxymethanamine hydrochloride (8.19 g, 83.9 mmol) were successively added. The mixture was stirred at 0° C. for 15 min and allowed to warm up to ambient temperature over 16 h. The mixture was washed with a saturated aqueous solution of NaHCO₃ and the aqueous layer was extracted with DCM (2×). The combined organic layers were washed with a 1 N aqueous solution of HCl, water then brine, filtered through hydrophobic paper, and concentrated under reduced pressure to afford (tert-butyl 4-[methoxy(methyl) carbamoyl]piperidine-1-carboxylate as a yellow oil (15.4 g, 99% p., 87% y.). m/z [M+Na]⁺=295.4, ¹H NMR (400 MHz, DMSO-d₆): δ (ppm) 3.95 (d, J=13.1 Hz, 2H), 3.68 (s, 3H), 3.09 (s, 3H), 2.85 (t, J=11.5 Hz, 4H), 1.68-1.59 (m, 2H), 1.39 (d, J=2.1 Hz, 10H).

### Intermediate 32B: tert-butyl 4-(2-chlorobenzoyl) piperidine-1-carboxylate

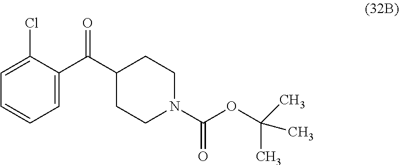 [SMILES: CC(C)(C)OC(=O)N1CCC(C(=O)c2ccccc2Cl)CC1]

In a three-neck round-bottom flask equipped with a thermometer, under argon, a solution of 1-bromo-2-chloro-benzene (3.5 mL, 29.4 mmol) in dry THF (75 mL) was cooled to −78° C. A 1.6 M solution of n-butyllithium in hexanes (20 mL, 32.1 mmol) was added dropwise and the mixture was stirred at −78° C. for 1.5 h. A solution of tert-butyl 4-[methoxy(methyl) carbamoyl]piperidine-1-carboxylate (7.50 g, 26.7 mmol) in dry THF (15 mL) was added dropwise, the mixture was stirred at −78° C. for 1 h, and then at ambient temperature for 16 h. A saturated aqueous solution of NH₄Cl was added dropwise. The mixture was stirred at ambient temperature for 10 min and was extracted with EtOAc (2×). The combined organic layers were washed with brine, dried over Na₂SO₄, filtered, and concentrated. The crude was purified by flash chromatography on silica gel using a gradient of EtOAc in cyclohexane from 5% to 50% to afford tert-butyl 4-(2-chlorobenzoyl) piperidine-1-carboxylate as a yellow oil (4.30 g, 44% p., 22% y.). m/z [M-tBu+2H]⁺=268.2.

Intermediate 32C: tert-butyl 4-[(2-chlorophenyl)-hydroxy-isothiazol-5-yl-methyl]piperidine-1-carboxylate

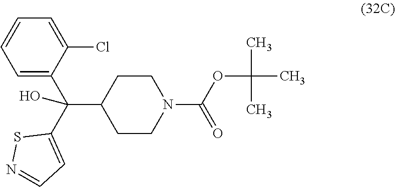 [SMILES: CC(C)(C)OC(=O)N1CCC(C(O)(c2ccns2)c2ccccc2Cl)CC1]

To a stirred solution of 5-bromo-1,2-thiazole (123 μL, 1.39 mmol) and tert-butyl 4-(2-chlorobenzoyl) piperidine-1-carboxylate (300 mg, 0.926 mmol) in anhydrous THF (6.7 mL) at −78° C. under argon, a 1.6 M solution of n-butyllithium (869 μL, 1.39 mmol) was slowly added (keeping the temperature below −70° C.). The reaction mixture was allowed to warm up to ambient temperature overnight. The reaction was quenched with the addition of a saturated aqueous solution of NH₄Cl. Water and EtOAc were added to the mixture. The layers were separated and the aqueous phase was extracted with EtOAc. The combined organic layers were washed with brine, dried over Na₂SO₄, filtered, and concentrated under reduced pressure. The crude was purified by flash chromatography on silica gel using a gradient of EtOAc in n-heptane from 5% to 40%. The fractions were concentrated under reduced pressure to afford tert-butyl 4-[(2-chlorophenyl)-hydroxy-isothiazol-5-yl-methyl]piperidine-1-carboxylate (172 mg, 94% p., 43% y.) as a white powder. m/z [M+Na]⁺=431.3, ¹H NMR (400 MHz, DMSO-d₆): δ (ppm) 8.39 (d, J=1.8 Hz, 1H), 7.96 (dd, J=8.0, 1.7 Hz, 1H), 7.42-7.33 (m, 3H), 7.29 (ddd, J=8.0, 7.0, 1.7 Hz, 1H), 6.31 (s, 1H), 4.10-3.86 (m, 2H), 3.26-3.11 (m, 1H), 2.77 (s, 2H), 1.66-1.39 (m, 2H), 1.37 (s, 9H), 1.25-1.10 (m, 2H).

### Intermediate 32D: tert-butyl 4-[chloro-(2-chlorophenyl)-isothiazol-5-yl-methyl]piperidine-1-carboxylate

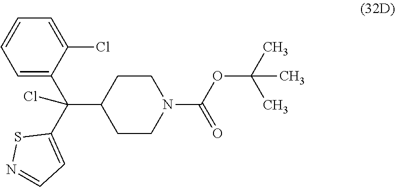 [SMILES: CC(C)(C)OC(=O)N1CCC(C(Cl)(c2ccns2)c2ccccc2Cl)CC1]

To a stirred solution of tert-butyl 4-[(2-chlorophenyl)-hydroxy-isothiazol-5-yl-methyl]piperidine-1-carboxylate (170 mg, 0.391 mmol) in dry THF (2.3 mL) at 0° C. under nitrogen was added pyridine (221 μL, 2.74 mmol) dropwise. The solution was stirred at 0° C. for 30 min. Thionyl chloride (105 μL, 1.37 mmol) was added dropwise and the reaction mixture was stirred at 0° C. for 2 h. The reaction was quenched with the addition of a saturated aqueous solution of NH₄Cl. EtOAc was added to the mixture. The layers were separated and the aqueous layer was extracted with EtOAc (2×). The combined organic layers were washed with brine, dried using a phase separator cartridge, and concentrated under reduced pressure to afford tert-butyl 4-[chloro-(2-chlorophenyl)-isothiazol-5-yl-methyl]piperidine-1-carboxylate as a yellow powder (182 mg, 63% p., 69% y.). m/z [M-tBu+2H]⁺=371.2.

### Intermediate 32E: 5-[(2-chlorophenyl)-(4-piperidyl)methyl]isothiazole hydrochloride

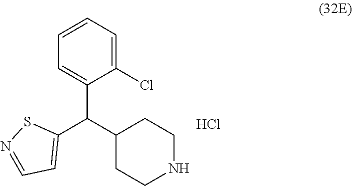 [SMILES: Clc1ccccc1C(c1ccns1)C1CCNCC1 ; Cl]

A sealed vial was charged with a solution of tert-butyl 4-[chloro-(2-chlorophenyl)-isothiazol-5-yl-methyl]piperidine-1-carboxylate (180 mg, 0.421 mmol) in AcOH (4.0 mL). Zinc dust (110 mg, 1.68 mmol) was added. The reaction mixture was stirred at 100° C. for 45 min. The reaction was quenched with the addition of a saturated aqueous solution of NaHCO₃(pH=10). EtOAc was added to the mixture. The layers were separated, and the aqueous layer extracted with EtOAc (2×). The combined organic layers were washed with brine, dried over Na₂SO₄, filtered, and concentrated under reduced pressure. The crude was diluted with a 2 M solution of HCl in Et₂O (1.3 mL, 2.65 mmol) and the reaction mixture was stirred at ambient temperature for 16 h. Next, a 4 M solution of HCl (1.1 mL, 4.21 mmol) was added and the reaction mixture was stirred for 16 h at 40° C. The reaction mixture was concentrated under reduced pressure to afford 5-[(2-chlorophenyl)-(4-piperidyl)methyl]isothiazole hydrochloride as a white powder (144 mg, 73% p., 76% y.). m/z [M+H]⁺=293.2.

### Intermediate 32F: tert-butyl(4S)-5-amino-4-[5-[4-[(2-chlorophenyl)-isothiazol-5-yl-methyl]piperidine-1-carbonyl]-6-methyl-1-oxo-isoindolin-2-yl]-5-oxo-pentanoate

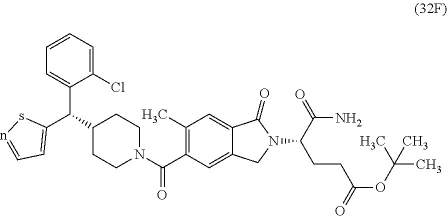

In a round-bottom flask, to a solution of 2-[(1S)-4-tert-butoxy-1-carbamoyl-4-oxo-butyl]-6-methyl-1-oxo-isoindoline-5-carboxylic acid (117 mg, 0.310 mmol) in DMF (500 μL) was added successively 5-[(2-chlorophenyl)-(4-piperidyl)methyl]isothiazole hydrochloride (140 mg, 0.310 mmol), HATU (200 mg, 0.510 mmol), and DIPEA (163 μL, 0.931 mmol). The mixture was stirred at ambient temperature overnight. Water was added and the mixture was extracted with EtOAc. The organic layer was washed with water, with a saturated aqueous solution of NaHCO₃ and brine. The organic layer was dried over Na₂SO₄, filtered, and concentrated under reduced pressure. The residue was purified by flash chromatography on silica gel using a gradient of EtOAc/EtOH (3/1) in cyclohexane from 0% to 100%. Selected fractions were combined and concentrated under reduced pressure to afford tert-butyl(4S)-5-amino-4-[5-[4-[(2-chlorophenyl)-isothiazol-5-yl-methyl]piperidine-1-carbonyl]-6-methyl-1-oxo-isoindolin-2-yl]-5-oxo-pentanoate as a yellow oil (110 mg, 89% p., 48% y.). m/z [M+Na]⁺=673.3.

### Example 32

In a sealed vial, to a solution of tert-butyl(4S)-5-amino-4-[5-[4-[(2-chlorophenyl)-isothiazol-5-yl-methyl]piperidine-1-carbonyl]-6-methyl-1-oxo-isoindolin-2-yl]-5-oxo-pentanoate (124 mg, 0.169 mmol) in acetonitrile (1.0 mL) was added 4-methylbenzenesulfonic acid hydrate (96.4 mg, 0.507 mmol). The solution was stirred at 90° C. for 1 h. The mixture was diluted with EtOAc and washed with a saturated aqueous solution of NaHCO₃ and brine. The organic layer was dried over Na₂SO₄, filtered, and concentrated under reduced pressure. The crude was purified by reverse-phase preparative chromatography using a gradient of acetonitrile in water from 0% to 100% (0.1% AcOH). The desired fractions were combined and concentrated under reduced pressure until most of the acetonitrile was concentrated under reduced pressure. A saturated aqueous solution of NaHCO₃ was added. The resulting suspension was filtered and dried at 50° C. under reduced pressure overnight. The resulting powder was purified by flash chromatography on silica gel using a gradient of EtOAc/EtOH (3/1) in cyclohexane from 0% to 100%. The desired fractions were combined and concentrated under reduced pressure. The residue was solubilized in a minimum amount of acetonitrile and was poured onto water. The resulting suspension was filtered and dried under at 50° C. under reduced pressure overnight to afford (3S)-3-[5-[4-[(2-chlorophenyl)-isothiazol-5-yl-methyl]piperidine-1-carbonyl]-6-methyl-1-oxo-isoindolin-2-yl]piperidine-2,6-dione as a white powder (12.6 mg, 96.0% p., 12% y.). m/z [M+H]⁺=577.2, ¹H NMR (500 MHz, DMSO-d₆): δ (ppm) 10.98 (s, 1H), 8.53-8.36 (m, 1H), 7.75-7.66 (m, 1H), 7.65-7.60 (m, 1H), 7.51-7.34 (m, 4H), 7.32-7.23 (m, 1H), 5.17-5.04 (m, 1H), 4.71-4.23 (m, 4H), 3.27-2.71 (m, 4H), 2.64-2.55 (m, 2H), 2.47-2.21 (m, 4H), 2.00 (br s, 1H), 1.77-0.88 (m, 5H).

### Example 33

### (3S)-3-[5-[4-[(S)-[2-chloro-3-[(3R)-1-methylpyrrolidin-3-yl]oxy-phenyl]-phenyl-methyl]piperidine-1-carbonyl]-6-methyl-1-oxo-isoindolin-2-yl]piperidine-2,6-dione hydrochloride

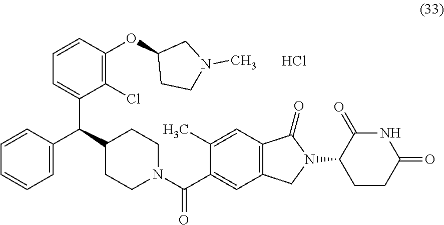 [SMILES: Cc1cc2c(cc1C(=O)N1CCC([C@@H](c3ccccc3)c3cccc(O[C@@H]4CCN(C)C4)c3Cl)CC1)CN([C@H]1CCC(=O)NC1=O)C2=O ; Cl]

### Intermediate 33A: benzyl 4-[(S)-[2-chloro-3-[(3R)-1-methylpyrrolidin-3-yl]oxy-phenyl]-phenyl-methyl]piperidine-1-carboxylate

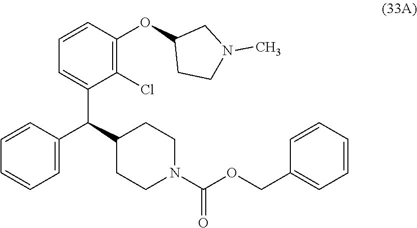

In a sealed vial, to a stirred solution of benzyl 4-[(S)-(2-chloro-3-hydroxy-phenyl)-phenyl-methyl]piperidine-1-carboxylate (250 mg, 0.573 mmol) in THF (2.9 mL) were added successively (3S)-1-methylpyrrolidin-3-ol (91.6 mg, 0.860 mmol), triphenylphosphane (226 mg, 0.860 mmol), and then DIAD (173 μL, 0.860 mmol). The reaction mixture was stirred at ambient temperature overnight. The reaction mixture was concentrated under reduced pressure. The crude was purified by flash chromatography on silica gel using a gradient of MeOH (+10% NH₃ 0.7 N in MeOH) in DCM from 1% to 10%. The desired fractions were combined and concentrated under reduced pressure to afford 4-[(S)-[2-chloro-3-[(3R)-1-methylpyrrolidin-3-yl]oxy-phenyl]-phenyl-methyl]piperidine-1-carboxylate as an off-white foam (265 mg, 99% p., 88% y.). m/z [M+H]⁺=519.3, ¹H NMR (400 MHz, DMSO-d₆): δ (ppm) 7.38-7.30 (m, 7H), 7.30-7.23 (m, 4H), 7.20-7.11 (m, 1H), 6.89-6.80 (m, 1H), 5.05 (s, 2H), 4.84 (ddt, J=8.8, 5.9, 2.8 Hz, 1H), 4.16 (d, J=11.2 Hz, 1H), 3.96 (d, J=13.2 Hz, 2H), 2.78 (dd, J=10.5, 6.0 Hz, 3H), 2.67-2.57 (m, 2H), 2.47 (d, J=19.8 Hz, 1H), 2.36-2.29 (m, 1H), 2.25 (s, 4H), 1.77-1.66 (m, 1H), 1.46 (d, J=13.1 Hz, 1H), 1.40-1.33 (m, 1H), 1.09-0.90 (m, 2H), [α]_{D}=−64°·mL·dm⁻¹ g⁻¹[20° C., Na lamp, 589 nm, C=1.3 mg/mL, MeOH].

### Intermediate 33B: 4-[(S)-[2-chloro-3-[(3R)-1-methylpyrrolidin-3-yl]oxy-phenyl]-phenyl-methyl]piperidine dihydroiodide

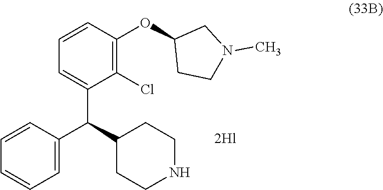

In a round-bottom flask, to a stirred solution of benzyl 4-[(S)-[2-chloro-3-[(3R)-1-methylpyrrolidin-3-yl]oxy-phenyl]-phenyl-methyl]piperidine-1-carboxylate (264 mg, 0.503 mmol) in dry acetonitrile (5.0 mL) at ambient temperature under nitrogen was added iodo (trimethyl) silane (215 μL, 1.51 mmol) dropwise. The reaction mixture was stirred at ambient temperature for 1 h. The mixture was cooled to 0° C. and MeOH (244 μL, 6.03 mmol) was added. The mixture was stirred at ambient temperature for 1 h and was concentrated under reduced pressure. Et₂O was added and the resulting mixture was stirred at ambient temperature for 1 h. The solid was filtered, washed with Et₂O, and dried under reduced pressure at 50° C. to afford 4-[(S)-[2-chloro-3-[(3R)-1-methylpyrrolidin-3-yl]oxy-phenyl]-phenyl-methyl]piperidine dihydroiodide as a yellow solid (376 mg, 70% p., 82% y.). m/z [M+H]⁺=385.3, [α]_{D}=−55°·mL·dm⁻¹ g⁻¹[20° C., Na lamp, 589 nm, C=1.3 mg/mL, MeOH].

### Intermediate 33C: tert-butyl(4S)-5-amino-4-[5-[4-[(S)-[2-chloro-3-[(3R)-1-methylpyrrolidin-3-yl]oxy-phenyl]-phenyl-methyl]piperidine-1-carbonyl]-6-methyl-1-oxo-isoindolin-2-yl]-5-oxo-pentanoate

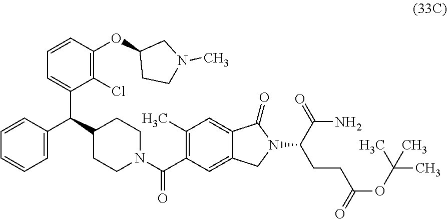 [SMILES: Cc1cc2c(cc1C(=O)N1CCC([C@@H](c3ccccc3)c3cccc(O[C@@H]4CCN(C)C4)c3Cl)CC1)CN([C@@H](CCC(=O)OC(C)(C)C)C(N)=O)C2=O]

In a round-bottom flask, to a stirred suspension of 4-[(S)-[2-chloro-3-[(3R)-1-methylpyrrolidin-3-yl]oxy-phenyl]-phenyl-methyl]piperidine dihydroiodide (357 mg, 0.391 mmol) in DMF (1.8 mL) were added successively 2-[(1S)-4-tert-butoxy-1-carbamoyl-4-oxo-butyl]-6-methyl-1-oxo-isoindoline-5-carboxylic acid (140 mg, 0.372 mmol), DIPEA (325 μL, 1.86 mmol), and then HATU (216 mg, 0.558 mmol). The reaction mixture was stirred at ambient temperature for 1 h. The reaction was quenched with the addition of water and basified with a saturated aqueous solution of NaHCO₃. EtOAc and then FastWorX® powder were added. The organic layer was concentrated under reduced pressure. The reaction mixture was filtered through a dry-loading cartridge which was then rinsed with water. The crude was purified by flash chromatography on silica gel using a gradient of EtOAc/EtOH (3/1) (+0.5% of NH_{3 7} N in MeOH) in cyclohexane from 50% to 100% to afford tert-butyl(4S)-5-amino-4-[5-[4-[(S)-[2-chloro-3-[(3R)-1-methylpyrrolidin-3-yl]oxy-phenyl]-phenyl-methyl]piperidine-1-carbonyl]-6-methyl-1-oxo-isoindolin-2-yl]-5-oxo-pentanoate as a beige solid (114 mg, 92% p., 38% y.). m/z [M+H]⁺=743.4. [α]_{D}=−43°·mL·dm⁻¹ g⁻¹[20° C., Na lamp, 589 nm, C=1.1 mg/mL, EtOH].

### Example 33

To a stirred solution of tert-butyl(4S)-5-amino-4-[5-[4-[(S)-[2-chloro-3-[(3R)-1-methylpyrrolidin-3-yl]oxy-phenyl]-phenyl-methyl]piperidine-1-carbonyl]-6-methyl-1-oxo-isoindolin-2-yl]-5-oxo-pentanoate (120 mg, 0.148 mmol) in acetonitrile (1.5 mL) was added 4-methylbenzenesulfonic acid hydrate (141 mg, 0.740 mmol). The reaction mixture was stirred at 80° C. for 4 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure. The residue was solubilized in 1,4-dioxane (4.0 mL). A 4 M solution of HCl in 1,4-dioxane (400 μL) was added dropwise and the reaction mixture was stirred for 30 min. Et₂O (40 mL) was added dropwise and the reaction mixture was stirred overnight. The resulting precipitate was filtered, washed with Et₂O, and dried under reduced pressure at 50° C. overnight to afford (35)-3-[5-[4-[(S)-[2-chloro-3-[(3R)-1-methylpyrrolidin-3-yl]oxy-phenyl]-phenyl-methyl]piperidine-1-carbonyl]-6-methyl-1-oxo-isoindolin-2-yl]piperidine-2,6-dione hydrochloride as a white powder (51.1 mg, 98.4% p., 48% y.). m/z [M+H]⁺=669.4, ¹H NMR (500 MHz, DMSO-d₆): δ (ppm) 10.98 (br d, J=7.8 Hz, 1H), 10.79-9.92 (m, 1H), 7.62 (br d, J=5.5 Hz, 1H), 7.48-7.22 (m, 7H), 7.22-7.10 (m, 1H), 7.04-6.93 (m, 1H), 5.17-5.06 (m, 2H), 4.58-4.48 (m, 1H), 4.47-4.37 (m, 1H), 4.34-4.18 (m, 2H), 3.81-3.41 (m, 3H), 3.30-3.17 (m, 2H), 3.07-2.71 (m, 6H), 2.66-2.56 (m, 2H), 2.44-2.30 (m, 3H), 2.25 (s, 2H), 2.17-2.04 (m, 1H), 1.99 (br s, 1H), 1.71-0.88 (m, 4H), [α]_{D}=−52°·mL·dm⁻¹.g⁻¹[20° C., Na lamp, 589 nm, C=1.4 mg/mL, DMSO].

### Example 34

### (3S)-3-[5-[4-[(R)-[2-chloro-3-[(3S)-1-methylpyrrolidin-3-yl]oxy-phenyl]-phenyl-methyl]piperidine-1-carbonyl]-6-methyl-1-oxo-isoindolin-2-yl]piperidine-2,6-dione hydrochloride

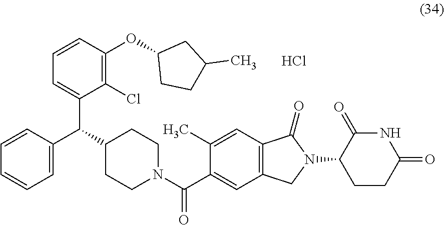 [SMILES: Cc1cc2c(cc1C(=O)N1CCC([C@H](c3ccccc3)c3cccc(O[C@H]4CCC(C)C4)c3Cl)CC1)CN([C@H]1CCC(=O)NC1=O)C2=O ; Cl]

Intermediate 34A: benzyl 4-[(R)-[2-chloro-3-[(3S)-1-methylpyrrolidin-3-yl]oxy-phenyl]-phenyl-methyl]piperidine-1-carboxylate

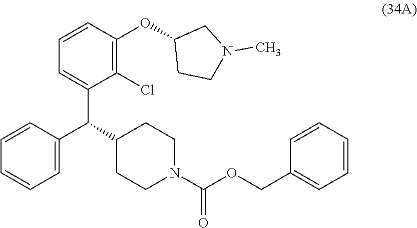 [SMILES: CN1CC[C@H](Oc2cccc([C@@H](c3ccccc3)C3CCN(C(=O)OCc4ccccc4)CC3)c2Cl)C1]

To a stirred solution of benzyl 4-[(R)-(2-chloro-3-hydroxy-phenyl)-phenyl-methyl]piperidine-1-carboxylate (250 mg, 0.573 mmol) in THF (2.9 mL) were added successively (3R)-1-methylpyrrolidin-3-ol (89.7 mg, 0.860 mmol), triphenylphosphane (226 mg, 0.860 mmol), and then DIAD (173 μL, 0.860 mmol). The reaction mixture was stirred at ambient temperature overnight. Additional (3R)-1-methylpyrrolidin-3-ol (89.7 mg, 0.860 mmol), triphenylphosphane (226 mg, 0.860 mmol), and DIAD (173 μL, 0.860 mmol) were added. The reaction mixture was stirred at ambient temperature overnight. The reaction mixture was concentrated under reduced pressure. The crude was purified by flash chromatography on silica gel using a gradient of EtOAc/EtOH (3/1) (+0.5% of NH₃ 7 N in MeOH) in n-heptane from 30% to 100% to afford benzyl 4-[(R)-[2-chloro-3-[(3S)-1-methylpyrrolidin-3-yl]oxy-phenyl]-phenyl-methyl]piperidine-1-carboxylate as a white foam (231 mg, 82% p., 64% y.). m/z [M+H]⁺=519.3, ¹H NMR (400 MHz, DMSO-d₆): δ (ppm) 7.43-7.20 (m, 12H), 7.19-7.12 (m, 1H), 6.84 (dd, J=6.0, 3.7 Hz, 1H), 5.04 (s, 2H), 4.88-4.78 (m, 1H), 4.15 (d, J=11.2 Hz, 1H), 3.95 (d, J=13.2 Hz, 2H), 2.77 (td, J=11.5, 4.4 Hz, 1H), 2.61 (d, J=11.3 Hz, 2H), 2.45 (d, J=11.1 Hz, 1H), 2.34 (q, J=7.5 Hz, 2H), 2.30-2.13 (m, 3H), 1.72 (dd, J=15.8, 9.1 Hz, 1H), 1.46 (d, J=13.1 Hz, 1H), 1.37 (d, J=14.4 Hz, 1H), 1.29-1.10 (m, 1H), 0.99 (dqd, J=24.8, 12.1, 4.2 Hz, 2H), [α]_{D}=+53°·mL·dm⁻¹.g⁻¹[20° C., Na lamp, 589 nm, C=0.7 mg/mL, MeOH].

### Intermediate 34B: 4-[(R)-[2-chloro-3-[(3S)-1-methylpyrrolidin-3-yl]oxy-phenyl]-phenyl-methyl]piperidine dihydroiodide

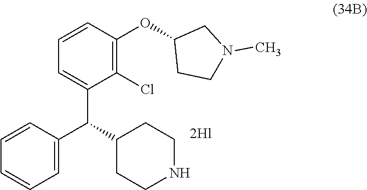 [SMILES: CN1CC[C@H](Oc2cccc([C@@H](c3ccccc3)C3CCNCC3)c2Cl)C1]

To a stirred solution of benzyl 4-[(R)-[2-chloro-3-[(3S)-1-methylpyrrolidin-3-yl]oxy-phenyl]-phenyl-methyl]piperidine-1-carboxylate (253 mg, 0.400 mmol) in acetonitrile (4.0 mL) at ambient temperature under nitrogen was added iodo (trimethyl) silane (171 μL, 1.20 mmol) dropwise. The reaction mixture was stirred at ambient temperature for 1 h. The mixture was cooled to 0° C. and MeOH (194 μL, 4.80 mmol) was added dropwise. The mixture was stirred at ambient temperature for 1 h and was concentrated under reduced pressure. Et₂O was added and the resulting mixture was stirred at ambient temperature overnight. The solid was filtered, washed with Et₂O, and dried under reduced pressure to afford 4-[(R)-[2-chloro-3-[(3S)-1-methylpyrrolidin-3-yl]oxy-phenyl]-phenyl-methyl]piperidine dihydroiodide as an orange solid (247 mg, 78% p., 75% y.). m/z [M+H]⁺=385.4. [α]_{D}=+40°·mL·dm⁻¹ g⁻¹[20° C., Na lamp, 589 nm, C=3.0 mg/mL, MeOH].

### Intermediate 34C: tert-butyl(4S)-5-amino-4-[5-[4-[(R)-[2-chloro-3-[(3S)-1-methylpyrrolidin-3-yl]oxy-phenyl]-phenyl-methyl]piperidine-1-carbonyl]-6-methyl-1-oxo-isoindolin-2-yl]-5-oxo-pentanoate

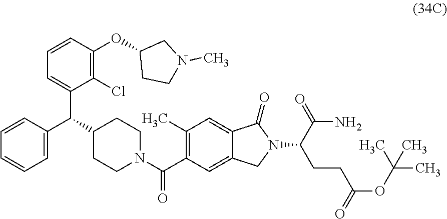 [SMILES: Cc1cc2c(cc1C(=O)N1CCC([C@H](c3ccccc3)c3cccc(O[C@H]4CCN(C)C4)c3Cl)CC1)CN([C@@H](CCC(=O)OC(C)(C)C)C(N)=O)C2=O]

To a stirred suspension of 2-[(1S)-4-tert-butoxy-1-carbamoyl-4-oxo-butyl]-6-methyl-1-oxo-isoindoline-5-carboxylic acid (190 mg, 0.480 mmol) in EtOAc (3.1 mL) and DCM (1.0 mL) were added successively DIPEA (420 μL, 2.41 mmol), 4-[(R)-[2-chloro-3-[(3S)-1-methylpyrrolidin-3-yl]oxy-phenyl]-phenyl-methyl]piperidine dihydroiodide (317 mg, 0.385 mmol), and then HATU (283 mg, 0.722 mmol). The reaction mixture was stirred at ambient temperature overnight. The reaction was quenched with the addition of a saturated aqueous solution of NaHCO₃. The aqueous layer was extracted with EtOAc (3×). The combined organic layers were washed with brine, dried over hydrophobic paper, and concentrated under reduced pressure. The crude was purified by flash chromatography on silica gel using a gradient of EtOAc/EtOH (3/1) in n-heptane from 2% to 100% to afford tert-butyl(4S)-5-amino-4-[5-[4-[(R)-[2-chloro-3-[(3S)-1-methylpyrrolidin-3-yl]oxy-phenyl]-phenyl-methyl]piperidine-1-carbonyl]-6-methyl-1-oxo-isoindolin-2-yl]-5-oxo-pentanoate as a yellow foam (162 mg, 73% p., 41% y.). m/z [M+H]⁺=743.3. [α]_{D}=+21°·mL·dm⁻¹ g⁻¹[20° C., Na lamp, 589 nm, C=2.2 mg/mL, MeOH].

### Example 34

To a stirred solution of tert-butyl(4S)-5-amino-4-[5-[4-[(R)-[2-chloro-3-[(3S)-1-methylpyrrolidin-3-yl]oxy-phenyl]-phenyl-methyl]piperidine-1-carbonyl]-6-methyl-1-oxo-isoindolin-2-yl]-5-oxo-pentanoate (161 mg, 0.158 mmol) in acetonitrile (1.6 mL) was added 4-methylbenzenesulfonic acid hydrate (150 mg, 0.791 mmol). The reaction mixture was stirred at 80° C. for 4 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was solubilized in 1,4-dioxane (4.1 mL). A 4 M solution of HCl in 1,4-dioxane (410 μL) was added dropwise and the reaction mixture was stirred for 30 min. Et₂O (41 mL) was added dropwise and the reaction mixture was stirred for 2 h. The resulting precipitate was filtered, washed with Et₂O, and dried under reduced pressure at 50° C. overnight. The resulting salt was purified by reverse-phase preparative chromatography using a gradient of acetonitrile in water from 0% to 100% (0.1% AcOH). The desired fractions were combined and concentrated under reduced pressure. The solid was solubilized in 1,4-dioxane (4.1 mL). A 4 M solution of HCl in 1,4-dioxane (410 μL) was added dropwise and the mixture was stirred at ambient temperature for 30 min. Et₂O (41 mL) was then added, and the reaction mixture was stirred at ambient temperature for 1 h. The resulting precipitate was filtered, washed with Et₂O, and dried under reduced pressure at 50° C. overnight to afford (3S)-3-[5-[4-[(R)-[2-chloro-3-[(3S)-1-methylpyrrolidin-3-yl]oxy-phenyl]-phenyl-methyl]piperidine-1-carbonyl]-6-methyl-1-oxo-isoindolin-2-yl]piperidine-2,6-dione hydrochloride as a white powder (57.2 mg, 98.2% p., 50% y.). m/z [M+H]⁺=669.4. ¹H NMR (500 MHz, DMSO-d₆): δ (ppm) 10.97 (s, 1H), 10.67-9.83 (m, 1H), 7.66-7.58 (m, 1H), 7.45 (br d, J=9.6 Hz, 1H), 7.42-7.38 (m, 1H), 7.38-7.34 (m, 2H), 7.32 (br d, J=6.5 Hz, 1H), 7.30-7.21 (m, 2H), 7.21-7.10 (m, 1H), 7.04-6.89 (m, 1H), 5.24-4.96 (m, 2H), 4.59-4.46 (m, 1H), 4.45-4.36 (m, 1H), 4.35-4.25 (m, 1H), 4.24-4.14 (m, 1H), 3.57 (s, 2H), 3.41-3.35 (m, 1H), 3.28-3.16 (m, 2H), 3.05-2.68 (m, 6H), 2.66-2.54 (m, 2H), 2.48-2.39 (m, 1H), 2.36 (br s, 1H), 2.34-2.22 (m, 3H), 2.17-2.03 (m, 1H), 2.01-1.93 (m, 1H), 1.65-1.02 (m, 4H), [α]_{D}=−16°·mL·dm⁻¹.g^{-1 [20}° C., Na lamp, 589 nm, C=0.5 mg/mL, DMSO].

### Example 35

### (3S)-3-[5-[4-[(S)-[2-chloro-3-[(3S)-1-methylpyrrolidin-3-yl]oxy-phenyl]-phenyl-methyl]piperidine-1-carbonyl]-6-methyl-1-oxo-isoindolin-2-yl]piperidine-2,6-dione hydrochloride

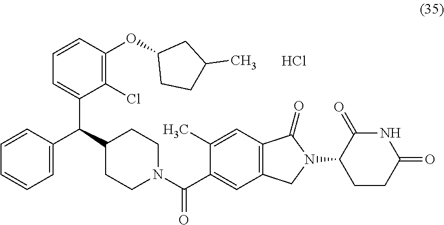 [SMILES: Cc1cc2c(cc1C(=O)N1CCC([C@@H](c3ccccc3)c3cccc(O[C@H]4CCC(C)C4)c3Cl)CC1)CN([C@H]1CCC(=O)NC1=O)C2=O ; Cl]

### Intermediate 35A: benzyl 4-[(S)-[2-chloro-3-[(3S)-1-methylpyrrolidin-3-yl]oxy-phenyl]-phenyl-methyl]piperidine-1-carboxylate

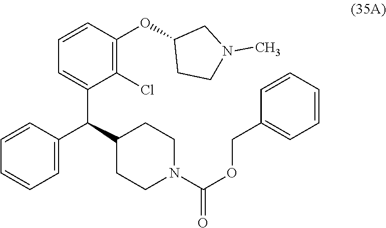 [SMILES: CN1CC[C@H](Oc2cccc([C@H](c3ccccc3)C3CCN(C(=O)OCc4ccccc4)CC3)c2Cl)C1]

To a stirred solution of benzyl 4-[(S)-(2-chloro-3-hydroxy-phenyl)-phenyl-methyl]piperidine-1-carboxylate (250 mg, 0.573 mmol) in THF (2.9 mL) were added successively (3R)-1-methylpyrrolidin-3-ol (94.5 μL, 0.860 mmol), triphenylphosphane (226 mg, 0.860 mmol), and DIAD (169 μL, 0.860 mmol). The reaction mixture was stirred at ambient temperature overnight. Additional (3R)-1-methylpyrrolidin-3-ol (189 μL, 1.72 mmol), triphenylphosphane (451 mg, 1.72 mmol), and DIAD (339 μL, 1.72 mmol) were added and the reaction mixture was stirred at ambient temperature overnight. Additional (3R)-1-methylpyrrolidin-3-ol (189 μL, 1.72 mmol), triphenylphosphane (451 mg, 1.72 mmol) and DIAD (339 μL, 1.72 mmol) were added and the reaction mixture was stirred at ambient temperature overnight. The reaction mixture was concentrated under reduced pressure. The crude was purified by flash chromatography on silica gel using a gradient of EtOAc in cyclohexane from 1% to 30% to afford benzyl 4-[(S)-[2-chloro-3-[(3S)-1-methylpyrrolidin-3-yl]oxy-phenyl]-phenyl-methyl]piperidine-1-carboxylate as a yellow gum (302 mg, 0.535 mmol, 92% p., 93% y.). m/z [M+H]⁺=519.3. [α]_{D}=−45°·mL·dm⁻¹ g⁻¹[20° C., Na lamp, 589 nm, C=2.8 mg/mL, EtOH].

### Intermediate 35B: 4-[(S)-[2-chloro-3-[(3S)-1-methylpyrrolidin-3-yl]oxy-phenyl]-phenyl-methyl]piperidine dihydroiodide

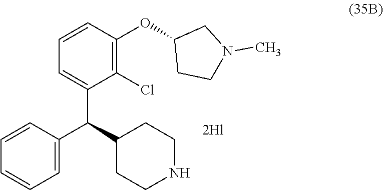 [SMILES: CN1CC[C@H](Oc2cccc([C@H](c3ccccc3)C3CCNCC3)c2Cl)C1]

To a stirred solution of benzyl 4-[(S)-[2-chloro-3-[(3S)-1-methylpyrrolidin-3-yl]oxy-phenyl]-phenyl-methyl]piperidine-1-carboxylate (294 mg, 0.566 mmol) in acetonitrile (5.7 mL) at ambient temperature under nitrogen was added iodo (trimethyl) silane (242 μL, 1.70 mmol) dropwise. The reaction mixture was stirred at ambient temperature for 30 min. The mixture was cooled to 0° C. and MeOH (275 μL, 6.80 mmol) was added dropwise. The mixture was stirred at ambient temperature for 1 h and was concentrated under reduced pressure. Et₂O was added and the resulting mixture was stirred at ambient temperature for 4 h. The solid was filtered, washed with Et₂O, and dried under reduced pressure to afford 4-[(S)-[2-chloro-3-[(3S)-1-methylpyrrolidin-3-yl]oxy-phenyl]-phenyl-methyl]piperidine dihydroiodide as a yellow solid (392 mg, 90% p., 97% y.). m/z [M+H]⁺=385.4. [α]_{D}=−30°·mL·dm⁻¹ g⁻¹[20° C., Na lamp, 589 nm, C=1.9 mg/mL, EtOH].

### Intermediate 35C: tert-butyl(4S)-5-amino-4-[5-[4-[(S)-[2-chloro-3-[(3S)-1-methylpyrrolidin-3-yl]oxy-phenyl]-phenyl-methyl]piperidine-1-carbonyl]-6-methyl-1-oxo-isoindolin-2-yl]-5-oxo-pentanoate

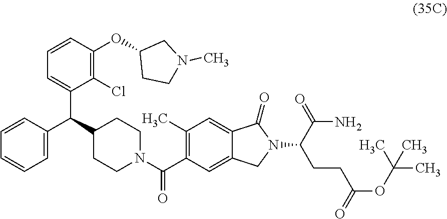 [SMILES: Cc1cc2c(cc1C(=O)N1CCC([C@@H](c3ccccc3)c3cccc(O[C@H]4CCN(C)C4)c3Cl)CC1)CN([C@@H](CCC(=O)OC(C)(C)C)C(N)=O)C2=O]

To a stirred solution of 2-[(1S)-4-tert-butoxy-1-carbamoyl-4-oxo-butyl]-6-methyl-1-oxo-isoindoline-5-carboxylic acid (195 mg, 0.518 mmol) in DMF (5.2 mL) were added successively DIPEA (452 μL, 2.59 mmol), 4-[(S)-[2-chloro-3-[(3S)-1-methylpyrrolidin-3-yl]oxy-phenyl]-phenyl-methyl]piperidine dihydroiodide (349 mg, 0.544 mmol), and then HATU (295 mg, 0.777 mmol). The reaction mixture was stirred at ambient temperature overnight. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by flash chromatography on silica gel using a gradient of EtOAc/EtOH (3/1) (0.5% conc. NH₄OH) in cyclohexane from 2% to 100% to afford tert-butyl(4S)-5-amino-4-[5-[4-[(S)-[2-chloro-3-[(3S)-1-methylpyrrolidin-3-yl]oxy-phenyl]-phenyl-methyl]piperidine-1-carbonyl]-6-methyl-1-oxo-isoindolin-2-yl]-5-oxo-pentanoate as a white solid (220 mg, 95% p., 54% y.). m/z [M+H]⁺=743.4. [α]_{D}=−33°·mL·dm⁻¹ g⁻¹[20° C., Na lamp, 589 nm, C=5.4 mg/mL, EtOH].

### Example 35

To a stirred solution of tert-butyl(4S)-5-amino-4-[5-[4-[(S)-[2-chloro-3-[(3S)-1-methylpyrrolidin-3-yl]oxy-phenyl]-phenyl-methyl]piperidine-1-carbonyl]-6-methyl-1-oxo-isoindolin-2-yl]-5-oxo-pentanoate (205 mg, 0.276 mmol) in acetonitrile (2.8 mL) was added 4-methylbenzenesulfonic acid hydrate (262 mg, 1.38 mmol). The reaction mixture was stirred at 80° C. for 4 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure and dried under reduced pressure. The residue was solubilized in 1,4-dioxane (6.9 mL). A 4 M solution of HCl in 1,4-dioxane (690 μL) was added dropwise. The reaction mixture was stirred for 30 min. Et₂O (76.0 mL) was added dropwise. The precipitate was triturated for 1 h, filtered, washed with Et₂O, and dried under reduced pressure at 50° C. overnight to afford (3S)-3-[5-[4-[(S)-[2-chloro-3-[(3S)-1-methylpyrrolidin-3-yl]oxy-phenyl]-phenyl-methyl]piperidine-1-carbonyl]-6-methyl-1-oxo-isoindolin-2-yl]piperidine-2,6-dione hydrochloride as a white solid (33.1 mg, 99.1% p., 17% y.). m/z [M+H]⁺=669.4, ¹H NMR (500 MHz, DMSO-d₆): δ (ppm) 10.98 (br d, J=7.1 Hz, 1H), 10.92-10.01 (m, 1H), 7.62 (br d, J=6.0 Hz, 1H), 7.51-7.42 (m, 1H), 7.41-7.22 (m, 6H), 7.21-7.11 (m, 1H), 7.06-6.88 (m, 1H), 5.19-5.06 (m, 2H), 4.53 (br s, 1H), 4.47-4.37 (m, 1H), 4.34-4.18 (m, 2H), 3.92-3.59 (m, 2H), 3.50-3.43 (m, 1H), 3.30-3.17 (m, 2H), 3.04-2.82 (m, 5H), 2.82-2.73 (m, 1H), 2.64-2.52 (m, 3H), 2.41-2.31 (m, 2H), 2.30-2.21 (m, 2H), 2.09 (br s, 1H), 2.03-1.95 (m, 1H), 1.67-0.89 (m, 4H), [α]_{D}=−37°. mL·dm⁻¹.g⁻¹[20° C., Na lamp, 589 nm, C=1.5 mg/mL, DMSO].

### Example 36

### N-[2-chloro-3-[1-[2-[(3S)-2,6-dioxo-3-piperidyl]-6-methyl-1-oxo-isoindoline-5-carbonyl]-4-piperidyl]-phenyl-methyl]phenyl]acetamide

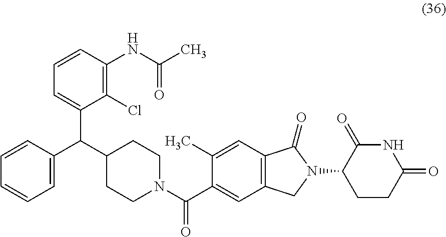 [SMILES: CC(=O)Nc1cccc(C(c2ccccc2)C2CCN(C(=O)c3cc4c(cc3C)C(=O)N([C@H]3CCC(=O)NC3=O)C4)CC2)c1Cl]

### Intermediate 36A: tert-butyl(4S)-4-[5-[4-[(3-acetamido-2-chloro-phenyl)-phenyl-methyl]piperidine-1-carbonyl]-6-methyl-1-oxo-isoindolin-2-yl]-5-amino-5-oxo-pentanoate

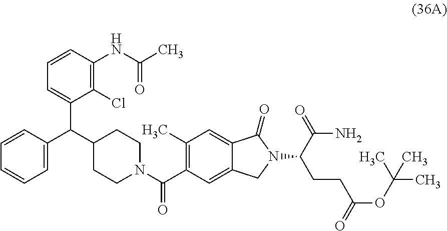 [SMILES: CC(=O)Nc1cccc(C(c2ccccc2)C2CCN(C(=O)c3cc4c(cc3C)C(=O)N([C@@H](CCC(=O)OC(C)(C)C)C(N)=O)C4)CC2)c1Cl]

To a stirred suspension of N-[2-chloro-3-[phenyl(4-piperidyl)methyl]phenyl]acetamide hydroiodide (60.0 mg, 0.127 mmol) in DCM (1.0 mL) were added successively 2-[(1S)-4-tert-butoxy-1-carbamoyl-4-oxo-butyl]-6-methyl-1-oxo-isoindoline-5-carboxylic acid (52.8 mg, 0.140 mmol), DIPEA (111 μL, 0.640 mmol), and a solution of T3P in EtOAc (50% w/w, 243 mg, 0.382 mmol). The reaction mixture was stirred at ambient temperature overnight. The reaction was quenched with the addition of a saturated aqueous solution of NaHCO₃. EtOAc was added to the mixture. The layers were separated, and the aqueous layer was extracted with EtOAc (2×). The combined organic layers were washed with brine, dried using a phase separator cartridge, and concentrated under reduced pressure. The crude was purified by flash chromatography on silica gel using a gradient of EtOAc/EtOH (3/1) in cyclohexane from 5% to 100%. The desired fractions were combined and concentrated under reduced pressure to afford tert-butyl(4S)-4-[5-[4-[(3-acetamido-2-chloro-phenyl)-phenyl-methyl]piperidine-1-carbonyl]-6-methyl-1-oxo-isoindolin-2-yl]-5-amino-5-oxo-pentanoate as a white solid (44.0 mg, 96% p., 47% y.). m/z [M+H]⁺=701.3.

### Example 36

In a sealed vial, to a solution of tert-butyl(4S)-4-[5-[4-[(3-acetamido-2-chloro-phenyl)-phenyl-methyl]piperidine-1-carbonyl]-6-methyl-1-oxo-isoindolin-2-yl]-5-amino-5-oxo-pentanoate (42.0 mg, 0.0575 mmol) in acetonitrile (2.0 mL) was added 4-methylbenzenesulfonic acid hydrate (32.8 mg, 0.172 mmol). The solution was stirred at 90° C. for 24 h. The mixture was diluted with EtOAc and washed with a saturated aqueous solution of NaHCO₃ and brine. The organic layer was dried over Na₂SO₄, filtered, and concentrated under reduced pressure. The crude was purified by reverse-phase preparative chromatography using a gradient of acetonitrile in water from 0% to 100% (0.1% AcOH). The desired fractions were combined and concentrated under reduced pressure until most of the acetonitrile evaporated. A saturated aqueous solution of NaHCO₃ was added. The resulting suspension was filtered and dried at 50° C. under reduced pressure overnight to afford (N-[2-chloro-3-[1-[2-[(3S)-2,6-dioxo-3-piperidyl]-6-methyl-1-oxo-isoindoline-5-carbonyl]-4-piperidyl]-phenyl-methyl]phenyl]acetamide as a white powder (1.26 mg, 98.4% p., 3% y.). m/z [M+H]⁺=627.3, ¹H NMR (600 MHz, DMSO-d₆): δ (ppm) 11.26-10.62 (m, 1H), 9.53-9.18 (m, 1H), 7.63-7.58 (m, 1H), 7.58-7.21 (m, 8H), 7.20-7.07 (m, 1H), 5.12-4.96 (m, 1H), 4.59-4.47 (m, 1H), 4.47-4.35 (m, 1H), 4.35-4.23 (m, 1H), 4.23-4.14 (m, 1H), 3.28-3.14 (m, 1H), 3.07-2.91 (m, 1H), 2.91-2.69 (m, 2H), 2.64-2.53 (m, 2H), 2.37-2.19 (m, 4H), 2.10-2.01 (m, 3H), 2.00-1.91 (m, 1H), 1.69-0.89 (m, 4H).

### Example 37

### (3S)-3-[5-[4-[(S)-[2-chloro-3-[(3R)-1-methylpyrrolidin-3-yl]methoxy]phenyl]-phenyl-methyl]piperidine-1-carbonyl]-6-methyl-1-oxo-isoindolin-2-yl]piperidine-2,6-dione hydrochloride

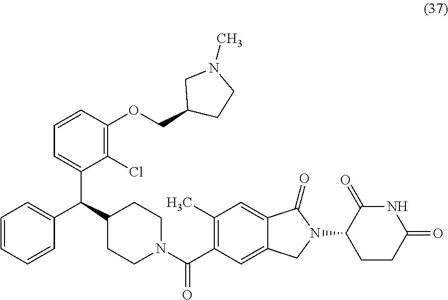 [SMILES: Cc1cc2c(cc1C(=O)N1CCC([C@@H](c3ccccc3)c3cccc(OC[C@@H]4CCN(C)C4)c3Cl)CC1)CN([C@H]1CCC(=O)NC1=O)C2=O]

### Intermediate 37A: tert-butyl(4S)-5-amino-4-[5-[4-[(S)-[2-chloro-3-[(3R)-1-methylpyrrolidin-3-yl]methoxy]phenyl]-phenyl-methyl]piperidine-1-carbonyl]-6-methyl-1-oxo-isoindolin-2-yl]-5-oxo-pentanoate

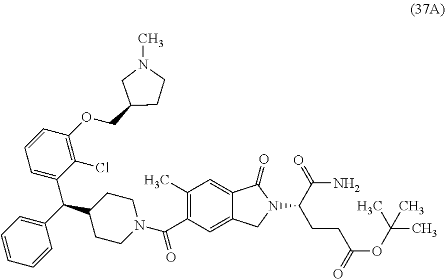 [SMILES: Cc1cc2c(cc1C(=O)N1CCC([C@@H](c3ccccc3)c3cccc(OC[C@@H]4CCN(C)C4)c3Cl)CC1)CN([C@@H](CCC(=O)OC(C)(C)C)C(N)=O)C2=O]

To a stirred solution of tert-butyl(4S)-5-amino-4-[5-[4-[(S)-(2-chloro-3-hydroxy-phenyl)-phenyl-methyl]piperidine-1-carbonyl]-6-methyl-1-oxo-isoindolin-2-yl]-5-oxo-pentanoate (143 mg, 0.216 mmol) in dry THF (1.5 mL) were added successively triphenylphosphane (170 mg, 0.649 mmol), [(3R)-1-methylpyrrolidin-3-yl]methanol (77.0 μL, 0.649 mmol), and then DIAD (128 μL, 0.649 mmol). The reaction mixture was stirred at ambient temperature for 72 h. The reaction mixture was concentrated under reduced pressure. The crude was purified by flash chromatography on silica gel using a gradient of EtOAc/EtOH (3/1) (0.5% conc. NH₄OH) in n-heptane from 5% to 100% to afford tert-butyl(4S)-5-amino-4-[5-[4-[(S)-[2-chloro-3-[(3R)-1-methylpyrrolidin-3-yl]methoxy]phenyl]-phenyl-methyl]piperidine-1-carbonyl]-6-methyl-1-oxo-isoindolin-2-yl]-5-oxo-pentanoate as an off-white foam (192 mg, 78% p., 91% y.). m/z [M+H]⁺=757.3. [α]_{D}=−30°·mL·dm⁻¹.g⁻¹[20° C., Na lamp, 589 nm, C=1.4 mg/mL, DMSO].

### Example 37

To a stirred solution of tert-butyl(4S)-5-amino-4-[5-[4-[(S)-[2-chloro-3-[(3R)-1-methylpyrrolidin-3-yl]methoxy]phenyl]-phenyl-methyl]piperidine-1-carbonyl]-6-methyl-1-oxo-isoindolin-2-yl]-5-oxo-pentanoate (193 mg, 0.255 mmol) in acetonitrile (2.5 mL) was added 4-methylbenzenesulfonic acid hydrate (242 mg, 1.27 mmol). The reaction mixture was stirred at 80° C. for 5 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated and dried under reduced pressure. The residue was solubilized in 1,4-dioxane (6.4 mL). A 4 M solution of HCl in 1,4-dioxane (640 μL) was added dropwise. The reaction mixture was stirred for 30 min. Et₂O (70 mL) was added dropwise. The precipitate was triturated for 1 h, filtered, washed with Et₂O and dried under reduced pressure at 50 C overnight to afford (3S)-3-[5-[4-[(S)-[2-chloro-3-[(3R)-1-methylpyrrolidin-3-yl]methoxy]phenyl]-phenyl-methyl]piperidine-1-carbonyl]-6-methyl-1-oxo-isoindolin-2-yl]piperidine-2,6-dione hydrochloride as an off-white solid (19.7 mg, 98.2% p., 11% y.). m/z [M+H]⁺=683.4, ¹H NMR (500 MHz, DMSO-d₆): δ (ppm) 11.16-10.84 (m, 1H), 10.68-9.86 (m, 1H), 7.61 (br s, 1H), 7.54-7.06 (m, 8H), 7.04-6.83 (m, 1H), 5.18-4.98 (m, 1H), 4.63-3.85 (m, 6H), 3.81-3.58 (m, 1H), 3.55-3.37 (m, 1H), 3.28-2.69 (m, 10H), 2.67-2.53 (m, 2H), 2.47-0.71 (m, 11H), [α]_{D}=+11°·mL·dm⁻¹ g⁻¹[20° C., Na lamp, 589 nm, C=1.0 mg/mL, DMSO].

### Example 38

### (3S)-3-[5-[4-[(S)-[2-chloro-3-[3-(dimethylamino)propoxy]phenyl]-phenyl-methyl]piperidine-1-carbonyl]-6-methyl-1-oxo-isoindolin-2-yl]piperidine-2,6-dione hydrochloride

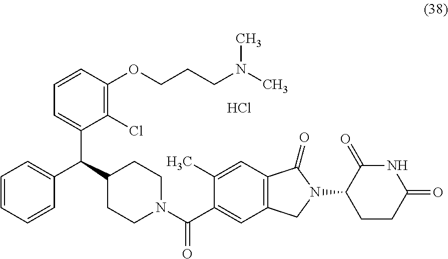 [SMILES: Cc1cc2c(cc1C(=O)N1CCC([C@@H](c3ccccc3)c3cccc(OCCCN(C)C)c3Cl)CC1)CN([C@H]1CCC(=O)NC1=O)C2=O ; Cl]

### Intermediate 38A: tert-butyl(4S)-5-amino-4-[5-[4-[(S)-[2-chloro-3-[3-(dimethylamino) propoxy]phenyl]-phenyl-methyl]piperidine-1-carbonyl]-6-methyl-1-oxo-isoindolin-2-yl]-5-oxo-pentanoate

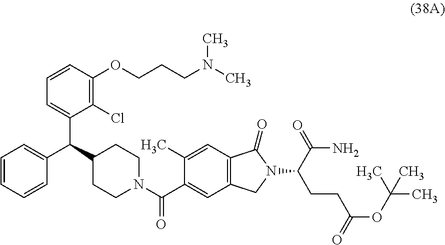 [SMILES: Cc1cc2c(cc1C(=O)N1CCC([C@@H](c3ccccc3)c3cccc(OCCCN(C)C)c3Cl)CC1)CN([C@@H](CCC(=O)OC(C)(C)C)C(N)=O)C2=O]

To a stirred solution of tert-butyl(4S)-5-amino-4-[5-[4-[(S)-(2-chloro-3-hydroxy-phenyl)-phenyl-methyl]piperidine-1-carbonyl]-6-methyl-1-oxo-isoindolin-2-yl]-5-oxo-pentanoate (143 mg, 0.216 mmol) in dry THF (1.5 mL) were added successively triphenylphosphane (170 mg, 0.649 mmol), 3-(dimethylamino)propan-1-ol (76.0 μL, 0.649 mmol), and then DIAD (128 μL, 0.649 mmol). The reaction mixture was stirred at ambient temperature for 72 h. The reaction mixture was concentrated under reduced pressure. The crude was purified by flash chromatography on silica gel using a gradient of EtOAc/EtOH (3/1) (0.5% conc. NH₄OH) in n-heptane from 5% to 100% to afford tert-butyl(4S)-5-amino-4-[5-[4-[(S)-[2-chloro-3-[3-(dimethylamino)propoxy]phenyl]-phenyl-methyl]piperidine-1-carbonyl]-6-methyl-1-oxo-isoindolin-2-yl]-5-oxo-pentanoate as a colorless oil (216 mg, 84% p., quant. yield)). m/z [M+H]⁺=745.3. [α]_{D}=−28°·mL·dm-1.g⁻¹[20° C., Na lamp, 589 nm, C=1.6 mg/mL, MeOH].

### Example 38

To a stirred solution of tert-butyl(4S)-5-amino-4-[5-[4-[(S)-[2-chloro-3-[3-(dimethylamino)propoxyphenyl]-phenyl]-methyl]piperidine-1-carbonyl]-6-methyl-1-oxo-isoindolin-2-yl]-5-oxo-pentanoate (216 mg, 0.290 mmol) in acetonitrile (2.9 mL) was added 4-methylbenzenesulfonic acid hydrate (276 mg, 1.45 mmol). The reaction mixture was stirred at 80° C. for 5 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure and dried under reduced pressure. The residue was solubilized in 1,4-dioxane (7.3 mL). A 4 M solution of HCl in 1,4-dioxane (730 μL) was added dropwise. The reaction mixture was stirred for 30 min. Et₂O (80 mL) was added dropwise. The precipitate was triturated for 1 h, filtered, washed with Et₂O and dried under reduced pressure at 50° C. overnight to afford (3S)-3-[5-[4-[(S)-[2-chloro-3-[3-(dimethylamino)propoxy]phenyl]-phenyl-methyl]piperidine-1-carbonyl]-6-methyl-1-oxo-isoindolin-2-yl]piperidine-2,6-dione hydrochloride as a white solid (51.9 mg, 98.5% p., 25% y.). m/z [M+H]⁺=671.4, ¹H NMR (600 MHz, DMSO-d₆): δ (ppm) 11.04-10.86 (m, 1H), 9.87-9.40 (m, 1H), 7.70-7.52 (m, 1H), 7.49-7.07 (m, 8H), 7.02-6.89 (m, 1H), 5.22-4.91 (m, 1H), 4.61-3.95 (m, 6H), 3.23-2.56 (m, 14H), 2.40-1.93 (m, 7H), 1.64-0.94 (m, 4H), [α]_{D}=−18°·mL·dm⁻¹ g⁻¹[20° C., Na lamp, 589 nm, C=1.6 mg/mL, DMSO].

### Example 39

### (3S)-3-[5-[4-[2-chloro-3-[2-(dimethylamino)ethyl]phenyl]-phenyl-methyl]piperidine-1-carbonyl]-6-methyl-1-oxo-isoindolin-2-yl]piperidine-2,6-dione hydrochloride

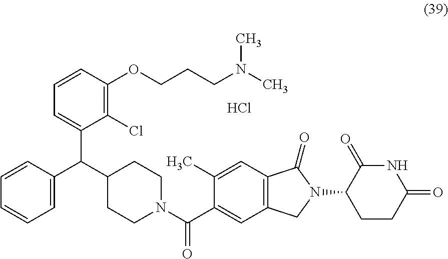 [SMILES: Cc1cc2c(cc1C(=O)N1CCC(C(c3ccccc3)c3cccc(OCCCN(C)C)c3Cl)CC1)CN([C@H]1CCC(=O)NC1=O)C2=O ; Cl]

### Intermediate 39A: benzyl 4-[2-chloro-3-[(E)-2-ethoxyvinyl]phenyl]-phenyl-methyl]piperidine-1-carboxylate

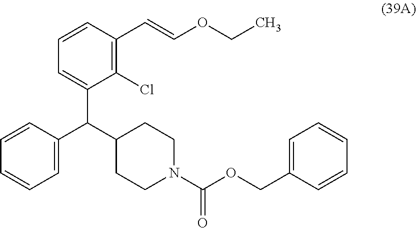 [SMILES: CCO/C=C/c1cccc(C(c2ccccc2)C2CCN(C(=O)OCc3ccccc3)CC2)c1Cl]

A sealed vial was successively charged with benzyl 4-[(3-bromo-2-chloro-phenyl)-phenyl-methyl]piperidine-1-carboxylate (340 mg, 0.634 mmol), 2-[(E)-2-ethoxyethenyl]-4,4,5,5-tetramethyl-1,3,2-dioxaborolane (256 mg, 1.27 mmol) and Cs₂CO₃ (632 mg, 1.90 mmol) in 1,4-dioxane (2.1 mL) and water (306 μL). The mixture was degassed 5 min with argon followed by the addition of Pd(dppf)Cl₂ (104 mg, 0.127 mmol) and the mixture was stirred at 100° C. overnight. The reaction mixture was filtered through talcum powder and the filtrate was concentrated under reduced pressure. The crude was purified by flash chromatography on silica gel using a gradient of EtOAc in n-heptane from 1% to 50%. Fractions were combined and concentrated under reduced pressure to afford benzyl 4-[[2-chloro-3-[(E)-2-ethoxyvinyl]phenyl]-phenyl-methyl]piperidine-1-carboxylate as a yellow oil (256 mg, 93% p., 77% y.). m/z [M+H]⁺=490.4, ¹H NMR (400 MHz, DMSO-d₆): δ (ppm) 7.48 (dd, J=7.7, 1.5 Hz, 1H), 7.39-7.20 (m, 11H), 7.18-7.14 (m, 1H), 7.12 (d, J=12.9 Hz, 1H), 6.03 (d, J=12.8 Hz, 1H), 5.05 (s, 2H), 4.17 (d, J=11.2 Hz, 1H), 3.96 (d, J=13.3 Hz, 2H), 3.90 (q, J=7.0 Hz, 2H), 2.78 (s, 3H), 2.49 (s, 1H), 1.50 (d, J=13.1 Hz, 1H), 1.36 (d, J=13.0 Hz, 1H), 1.24 (t, J=7.0 Hz, 3H), 1.07-0.98 (m, 1H).

### Intermediate 39B: benzyl 4-[[2-chloro-3-(2-oxoethyl)phenyl]-phenyl-methyl]piperidine-1-carboxylate

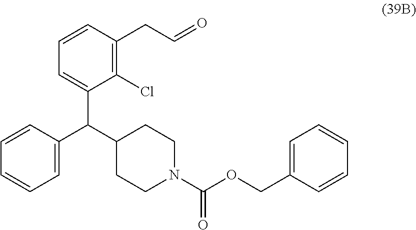 [SMILES: O=CCc1cccc(C(c2ccccc2)C2CCN(C(=O)OCc3ccccc3)CC2)c1Cl]

To a stirred solution of benzyl 4-[2-chloro-3-[(E)-2-ethoxyvinyl]phenyl]-phenyl-methyl]piperidine-1-carboxylate (254 mg, 0.482 mmol) in dry DCM (7.9 mL) under argon was added TFA (185 μL, 2.41 mmol) and the mixture was stirred at 0° C. for 30 min. The mixture was quenched with a saturated aqueous solution of NaHCO₃. The layers were separated, and the aqueous layer was extracted with DCM. The combined organic layers were washed with brine, dried over Na₂SO₄, filtered, and concentrated under reduced pressure. The crude was purified by flash chromatography on silica gel using a gradient of EtOAc in n-heptane from 1% to 100%. Fractions were combined and concentrated under reduced pressure to afford benzyl 4-[[2-chloro-3-(2-oxoethyl)phenyl]-phenyl-methyl]piperidine-1-carboxylate as a colorless gum (117 mg, 88% p., 46% y.). m/z [M+Na]⁺=484.3, ¹H NMR (400 MHz, DMSO-d₆): δ (ppm) 9.65 (d, J=1.4 Hz, 1H), 7.66 (dd, J=8.0, 1.7 Hz, 1H), 7.39-7.25 (m, 10H), 7.21 (dd, J=7.6, 1.6 Hz, 1H), 7.19-7.14 (m, 1H), 5.05 (s, 2H), 4.16 (d, J=11.2 Hz, 1H), 3.96 (d, J=13.4 Hz, 2H), 3.90 (d, J=1.4 Hz, 2H), 2.78 (s, 3H), 2.45 (d, J=11.3 Hz, 1H), 1.47 (d, J=13.3 Hz, 1H), 1.36 (d, J=13.0 Hz, 1H), 1.01 (dd, J=12.5, 4.1 Hz, 1H).

### Intermediate 39C: benzyl 4-[[2-chloro-3-[2-(dimethylamino)ethyl]phenyl]-phenyl-methyl]piperidine-1-carboxylate

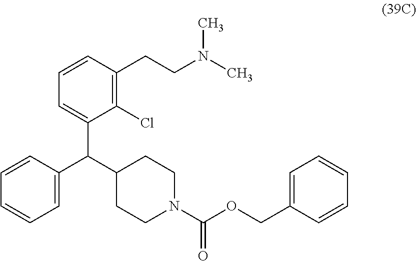 [SMILES: CN(C)CCc1cccc(C(c2ccccc2)C2CCN(C(=O)OCc3ccccc3)CC2)c1Cl]

In a sealed vial, to a stirred solution of benzyl 4-[[2-chloro-3-(2-oxoethyl)phenyl]-phenyl-methyl]piperidine-1-carboxylate (117 mg, 0.223 mmol) and a 2 M solution of N-methylmethanamine (123 μL, 0.245 mmol) in MeOH (2.2 mL) was added AcOH (12.8 μL, 0.223 mmol). The mixture was stirred at ambient temperature for 30 min and supported sodium cyanoborohydride (2.0 mmol/g, 223 mg, 0.446 mmol) was added. The mixture was stirred at ambient temperature overnight and was filtered. The residue was washed with MeOH, and the filtrate was concentrated. The crude was purified by reverse-phase preparative chromatography using a gradient of acetonitrile in water from 0% to 100% (0.1% AcOH). Acetonitrile was evaporated and a saturated aqueous solution of NaHCO₃ was added. The aqueous layer was extracted with DCM 3x). The combined organic layers were washed with brine, dried over Na₂SO₄, filtered, and concentrated under reduced pressure to afford benzyl 4-[2-chloro-3-[2-(dimethylamino)ethyl]phenyl]-phenyl-methyl]piperidine-1-carboxylate as a brown oil (50.0 mg, 87% p., 40% y.). m/z [M+H]⁺=491.4, ¹H NMR (400 MHz, DMSO-d₆): δ (ppm) 7.55 (dd, J=7.9, 1.7 Hz, 1H), 7.36-7.25 (m, 10H), 7.20-7.13 (m, 2H), 5.05 (s, 2H), 4.19 (d, J=11.2 Hz, 1H), 3.96 (d, J=13.2 Hz, 2H), 2.92-2.60 (m, 3H), 2.51-2.30 (m, 3H), 2.17 (s, 6H), 1.45 (d, J=13.2 Hz, 1H), 1.40-1.20 (m, 2H), 1.13-0.84 (m, 2H).

### Intermediate 39D: 2-[2-chloro-3-[phenyl(4-piperidyl)methyl]phenyl]-N,N-dimethyl-ethanamine dihydroiodide

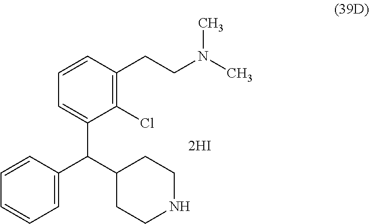 [SMILES: CN(C)CCc1cccc(C(c2ccccc2)C2CCNCC2)c1Cl]

To a stirred solution of benzyl 4-[[2-chloro-3-[2-(dimethylamino)ethyl]phenyl]-phenyl-methyl]piperidine-1-carboxylate (50.0 mg, 0.0886 mmol) in acetonitrile (900 L) at ambient temperature was added iodo (trimethyl) silane (37.8 μL, 0.266 mmol) dropwise. The reaction mixture was stirred at ambient temperature for 15 min. The mixture was cooled to 0° C. and MeOH (43.1 μL, 1.06 mmol) was added dropwise. The mixture was stirred at ambient temperature for 1 h and was concentrated under reduced pressure. Et₂O was added and the resulting mixture was stirred at ambient temperature for 1 h. The solid was filtered, washed with Et₂O, and dried under reduced pressure for 2 h to afford 2-[2-chloro-3-[phenyl(4-piperidyl)methyl]phenyl]-N,N-dimethyl-ethanamine dihydroiodide as a golden solid (60.0 mg, 76% p., 84% y.). m/z [M+H]⁺=357.4, ¹H NMR (400 MHz, DMSO-d₆): δ (ppm) 9.46 (s, 1H), 8.40 (s, 1H), 8.09 (s, 1H), 7.64 (dd, J=7.8, 1.6 Hz, 1H), 7.44-7.40 (m, 1H), 7.39-7.35 (m, 2H), 7.31-7.25 (m, 3H), 7.22-7.17 (m, 1H), 4.62 (s, 1H), 4.16 (d, J=11.3 Hz, 1H), 3.29-3.18 (m, 4H), 3.11-3.00 (m, 2H), 2.85 (d, J=4.8 Hz, 6H), 2.79 (d, J=13.2 Hz, 1H), 2.67-2.54 (m, 1H), 1.57 (d, J=14.4 Hz, 1H), 1.49 (d, J=14.0 Hz, 1H), 1.36-1.15 (m, 2H).

### Intermediate 39E: tert-butyl(4S)-5-amino-4-[5-[4-[[2-chloro-3-[2-(dimethylamino)ethyl]phenyl]-phenyl-methyl]piperidine-1-carbonyl]-6-methyl-1-oxo-isoindolin-2-yl]-5-oxo-pentanoate

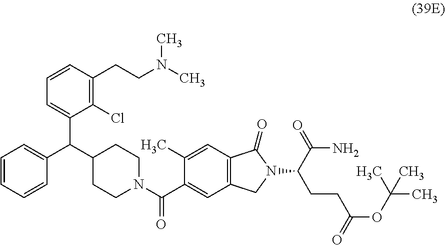 [SMILES: Cc1cc2c(cc1C(=O)N1CCC(C(c3ccccc3)c3cccc(CCN(C)C)c3Cl)CC1)CN([C@@H](CCC(=O)OC(C)(C)C)C(N)=O)C2=O]

To a solution of 2-[2-chloro-3-[phenyl(4-piperidyl)methyl]phenyl]-N,N-dimethyl-ethanamine dihydroiodide (60.0 mg, 0.0744 mmol) in DMF (400 μL), were added 2-[(1S)-4-tert-butoxy-1-carbamoyl-4-oxo-butyl]-6-methyl-1-oxo-isoindoline-5-carboxylic acid (29.4 mg, 0.0781 mmol), DIPEA (39.0 μL, 0.223 mmol) and HATU (43.3 mg, 0.112 mmol). The mixture was stirred at ambient temperature overnight. Water, EtOAc and FastWorkX® were added. The organic solvent was evaporated under reduced pressure. The mixture was filtered through a dry-loading cartridge and the resin was rinsed with water. The crude was purified by flash chromatography on silica gel using a gradient of MeOH in DCM from 0% to 20%. The desired fractions were combined and concentrated under reduced pressure to afford tert-butyl(4S)-5-amino-4-[5-[4-[2-chloro-3-[2-(dimethylamino)ethyl]phenyl]-phenyl-methyl]piperidine-1-carbonyl]-6-methyl-1-oxo-isoindolin-2-yl]-5-oxo-pentanoate as a yellow solid (22.0 mg, 65% p., 27% y.). m/z [M+H]⁺=715.5.

### Example 39

To a stirred solution of tert-butyl(4S)-5-amino-4-[5-[4-[2-chloro-3-[2-(dimethylamino)ethyl]phenyl]-phenyl-methyl]piperidine-1-carbonyl]-6-methyl-1-oxo-isoindolin-2-yl]-5-oxo-pentanoate (22.0 mg, 0.0308 mmol) in acetonitrile (310 μL) was added 4-methylbenzenesulfonic acid hydrate (29.3 mg, 0.154 mmol). The reaction mixture was stirred at 80° C. for 4 h. The reaction mixture was cooled to ambient temperature and quenched with a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated and dried under reduced pressure. The residue was solubilized in 1,4-dioxane (770 μL). A 4 M solution of HCl in 1,4-dioxane (77.0 μL) was added dropwise. The reaction mixture was stirred for 30 min. Et₂O (8.5 mL) was added dropwise. The precipitate was triturated for 1 h, filtered, washed with Et₂O and dried under reduced pressure at 50° C. overnight to afford (3S)-3-[5-[4-[[2-chloro-3-[2-(dimethylamino)ethyl]phenyl]-phenyl-methyl]piperidine-1-carbonyl]-6-methyl-1-oxo-isoindolin-2-yl]piperidine-2,6-dione hydrochloride as a pale yellow solid (2.20 mg, 89.9% p., 9% y.). m/z [M+H]⁺=641.3, ¹H NMR (600 MHz, DMSO-d₆): δ (ppm) 11.10-10.79 (m, 1H), 10.20-9.62 (m, 1H), 7.79-7.57 (m, 2H), 7.56-6.99 (m, 9H), 5.17-5.01 (m, 1H), 4.61-4.47 (m, 1H), 4.47-4.35 (m, 1H), 4.35-4.11 (m, 2H), 3.27-2.65 (m, 13H), 2.64-2.53 (m, 2H), 2.47-2.16 (m, 4H), 2.07-1.92 (m, 1H), 1.09 (t, J=7.0 Hz, 4H).

### Example 40

### (3S)-3-[5-[4-[(S)-[2-chloro-3-[(3-methylimidazol-4-yl)methoxy]phenyl]-phenyl-methyl]piperidine-1-carbonyl]-6-methyl-1-oxo-isoindolin-2-yl]piperidine-2,6-dione

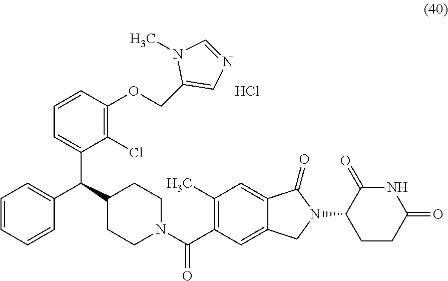 [SMILES: Cc1cc2c(cc1C(=O)N1CCC([C@@H](c3ccccc3)c3cccc(OCc4cncn4C)c3Cl)CC1)CN([C@H]1CCC(=O)NC1=O)C2=O ; Cl]

### Intermediate 40A: tert-butyl(4S)-5-amino-4-[5-[4-[(S)-[2-chloro-3-[(3-methylimidazol-4-yl)methoxy]phenyl]-phenyl-methyl]piperidine-1-carbonyl]-6-methyl-1-oxo-isoindolin-2-yl]-5-oxo-pentanoate

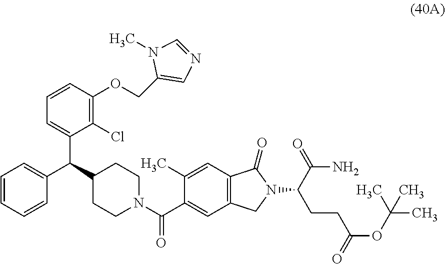 [SMILES: Cc1cc2c(cc1C(=O)N1CCC([C@@H](c3ccccc3)c3cccc(OCc4cncn4C)c3Cl)CC1)CN([C@@H](CCC(=O)OC(C)(C)C)C(N)=O)C2=O]

To a stirred solution of tert-butyl(4S)-5-amino-4-[5-[4-[(S)-(2-chloro-3-hydroxy-phenyl)-phenyl-methyl]piperidine-1-carbonyl]-6-methyl-1-oxo-isoindolin-2-yl]-5-oxo-pentanoate (143 mg, 0.216 mmol) in dry THF (1.5 mL) were added successively triphenylphosphane (170 mg, 0.649 mmol), (1-methyl-1H-imidazol-5-yl)methanol (74.2 mg, 0.649 mmol), and then DIAD (128 μL, 0.649 mmol). The reaction mixture was stirred at ambient temperature for 72 h. Additional (1-methyl-1H-imidazol-5-yl)methanol (74.2 mg, 0.649 mmol), triphenylphosphane (150 mg, 0.572 mmol), and DIAD (85.0 μL, 0.432 mmol) were added and the reaction mixture was stirred at 60° C. overnight. The reaction mixture was concentrated under reduced pressure. The crude was purified by flash chromatography on silica gel using a gradient of EtOAc/EtOH (3/1) (0.5% conc. NH₄OH) in n-heptane from 5% to 100% and then, by reverse-phase preparative chromatography using a gradient of acetonitrile in water from 5% to 100% to afford tert-butyl(4S)-5-amino-4-[5-[4-[(S)-[2-chloro-3-[(3-methylimidazol-4-yl)methoxy]phenyl]-phenyl-methyl]piperidine-1-carbonyl]-6-methyl-1-oxo-isoindolin-2-yl]-5-oxo-pentanoate as a yellow solid (42.0 mg, 64% p., 16% y.). m/z [M+H]⁺=754.3.

### Example 40

To a stirred solution of tert-butyl(4S)-5-amino-4-[5-[4-[(S)-[2-chloro-3-[(3-methylimidazol-4-yl)methoxy]phenyl]-phenyl-methyl]piperidine-1-carbonyl]-6-methyl-1-oxo-isoindolin-2-yl]-5-oxo-pentanoate (42.0 mg, 0.0557 mmol) in acetonitrile (560 μL) was added 4-methylbenzenesulfonic acid hydrate (53.0 mg, 0.278 mmol). The reaction mixture was stirred at 80° C. for 4 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated and dried under reduced pressure. The residue was solubilized in 1,4-dioxane (1.4 mL). A 4 M solution of HCl in 1,4-dioxane (140 μL) was added dropwise. The reaction mixture was stirred for 30 min. Et₂O (15 mL) was added dropwise. The precipitate was triturated for 1 h, filtered, washed with Et₂O, and dried under reduced pressure at 50° C. overnight to afford (3S)-3-[5-[4-[(S)-[2-chloro-3-[(3-methylimidazol-4-yl)methoxy]phenyl]-phenyl-methyl]piperidine-1-carbonyl]-6-methyl-1-oxo-isoindolin-2-yl]piperidine-2,6-dione hydrochloride as a white solid (12.4 mg, 99.0% p., 31% y.). m/z [M+H]⁺=680.3, ¹H NMR (600 MHz, DMSO-d₆): δ (ppm) 14.76-13.89 (m, 1H), 11.11-10.82 (m, 1H), 9.12-8.90 (m, 1H), 7.77 (s, 1H), 7.60 (br s, 1H), 7.43-7.35 (m, 3H), 7.35-7.28 (m, 3H), 7.23 (t, J=7.6 Hz, 1H), 7.21-7.18 (m, 1H), 7.13 (q, J=7.0 Hz, 1H), 5.34-5.22 (m, 2H), 5.13-5.05 (m, 1H), 4.57-4.12 (m, 4H), 3.96-3.81 (m, 3H), 3.24-2.55 (m, 6H), 2.45-2.20 (m, 4H), 1.99 (br d, J=7.8 Hz, 1H), 1.66-0.88 (m, 4H), [α]_{D}=+11°. mL·dm⁻¹.g⁻¹[20° C., Na lamp, 589 nm, C=1.0 mg/mL, DMSO].

### Example 41

### (3S)-3-[5-[4-[(R)-[2-chloro-3-[(3-methylimidazol-4-yl)methoxy]phenyl]-phenyl-methyl]piperidine-1-carbonyl]-6-methyl-1-oxo-isoindolin-2-yl]piperidine-2,6-dione

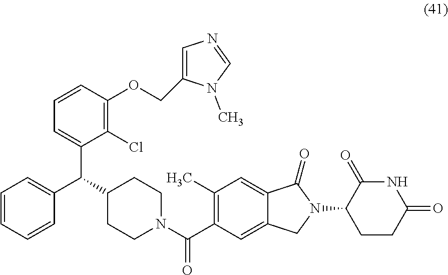 [SMILES: Cc1cc2c(cc1C(=O)N1CCC([C@H](c3ccccc3)c3cccc(OCc4cncn4C)c3Cl)CC1)CN([C@H]1CCC(=O)NC1=O)C2=O]

### Intermediate 41A: tert-butyl(4S)-5-amino-4-[5-[4-[(R)-[2-chloro-3-[(3-methylimidazol-4-yl)methoxy]phenyl]-phenyl-methyl]piperidine-1-carbonyl]-6-methyl-1-oxo-isoindolin-2-yl]-5-oxo-pentanoate

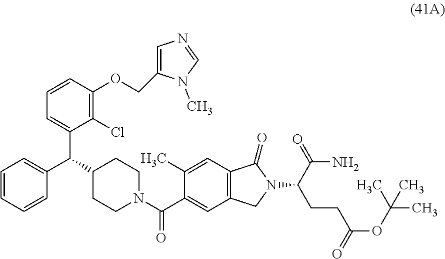 [SMILES: Cc1cc2c(cc1C(=O)N1CCC([C@H](c3ccccc3)c3cccc(OCc4cncn4C)c3Cl)CC1)CN([C@@H](CCC(=O)OC(C)(C)C)C(N)=O)C2=O]

To a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.227 mmol) in dry THF (1.5 mL) were added successively triphenylphosphane (179 mg, 0.682 mmol), (1-methyl-1H-imidazol-5-yl)methanol (67.2 μL, 0.682 mmol), and then DIAD (134 μL, 0.682 mmol). The reaction mixture was stirred at ambient temperature for 72 h. The reaction mixture was concentrated under reduced pressure. The crude was purified by flash chromatography on silica gel using a gradient of EtOAc/EtOH (3/1) (0.5% conc. NH₄OH) in n-heptane from 5% to 100% to afford tert-butyl(4S)-5-amino-4-[5-[4-[(R)-[2-chloro-3-[(3-methylimidazol-4-yl) methoxy]phenyl]-phenyl-methyl]piperidine-1-carbonyl]-6-methyl-1-oxo-isoindolin-2-yl]-5-oxo-pentanoate as an off-white foam (108 mg, 84% p., 53% y.). m/z [M+H]⁺=754.4.

### Example 41

In a sealed vial, a solution of tert-butyl(4S)-5-amino-4-[5-[4-[(R)-[2-chloro-3-[(3-methylimidazol-4-yl)methoxy]phenyl]-phenyl-methyl]piperidine-1-carbonyl]-6-methyl-1-oxo-isoindolin-2-yl]-5-oxo-pentanoate (106 mg, 0.141 mmol) and 4-methylbenzenesulfonic acid hydrate (80.2 mg, 0.422 mmol) in acetonitrile (1.0 mL) was stirred at 85° C. for 3 h. The mixture was cooled to ambient temperature then EtOAc and water were added. The mixture was poured into saturated aqueous NaHCO₃ and extracted with EtOAc (2×). The combined organic layers were washed with brine, filtered through hydrophobic paper, and concentrated. The crude was purified by reverse-phase preparative chromatography using a gradient of acetonitrile in water from 0% to 100% (0.2% AcOH). The desired fractions were combined, and acetonitrile evaporated. A saturated aqueous solution of NaHCO₃ was added and the mixture was extracted with EtOAc (2×). The combined organic layers were washed with brine, filtered through hydrophobic paper, and concentrated under reduced pressure. The residue was solubilized in DCM and added dropwise to stirred n-pentane. The resulting suspension was stirred at ambient temperature for 2 h and filtered. The residue was washed with n-pentane and dried under reduced pressure at 50° C. for 16 h to afford (3S)-3-[5-[4-[(R)-[2-chloro-3-[(3-methylimidazol-4-yl)methoxy]phenyl]-phenyl-methyl]piperidine-1-carbonyl]-6-methyl-1-oxo-isoindolin-2-yl]piperidine-2,6-dione as a white solid (29.0 mg, 98.9% p., 30% y.). m/z [M+H]⁺=680.4. ¹H NMR (500 MHz, DMSO-d₆): δ (ppm) 10.98 (s, 1H), 7.75-7.54 (m, 2H), 7.53-7.08 (m, 9H), 7.01 (d, J=17.1 Hz, 1H), 5.30-5.02 (m, 3H), 4.62-4.05 (m, 4H), 3.75-3.57 (m, 3H), 3.29-3.11 (m, 1H), 3.09-2.68 (m, 3H), 2.67-2.53 (m, 2H), 2.46-2.13 (m, 4H), 2.08-1.83 (m, 1H), 1.75-0.67 (m, 4H), [α]_{D}=+14°·mL·dm⁻¹ g⁻¹[20° C., Na lamp, 589 nm, C=1.4 mg/mL, DMSO].

### Example 42

### (3S)-3-[5-[4-[(R)-[2-chloro-3-(1-methylazetidin-3-yl)oxy-phenyl]-phenyl-methyl]piperidine-1-carbonyl]-6-methyl-1-oxo-isoindolin-2-yl]piperidine-2,6-dione

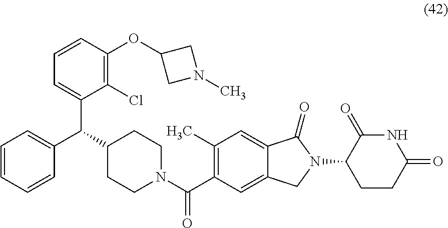 [SMILES: Cc1cc2c(cc1C(=O)N1CCC([C@H](c3ccccc3)c3cccc(OC4CN(C)C4)c3Cl)CC1)CN([C@H]1CCC(=O)NC1=O)C2=O]

### Intermediate 42A: tert-butyl(4S)-5-amino-4-[5-[4-[(R)-[2-chloro-3-(1-methylazetidin-3-yl)oxy-phenyl]-phenyl-methyl]piperidine-1-carbonyl]-6-methyl-1-oxo-isoindolin-2-yl]-5-oxo-pentanoate

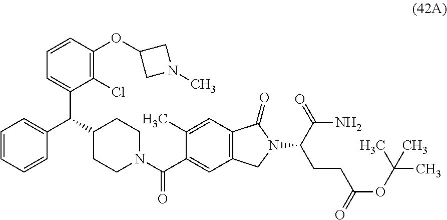 [SMILES: Cc1cc2c(cc1C(=O)N1CCC([C@H](c3ccccc3)c3cccc(OC4CN(C)C4)c3Cl)CC1)CN([C@@H](CCC(=O)OC(C)(C)C)C(N)=O)C2=O]

To a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.227 mmol) in dry THF (1.5 mL) were added successively triphenylphosphane (179 mg, 0.682 mmol), 1-methylazetidin-3-ol (56.1 μL, 0.682 mmol), and then DIAD (134 μL, 0.682 mmol). The reaction mixture was stirred at ambient temperature for 72 h. The reaction mixture was concentrated under reduced pressure. The crude was purified by flash chromatography on silica gel using a gradient of EtOAc/EtOH (3/1) (0.5% conc. NH₄OH) in n-heptane from 5% to 100% to afford tert-butyl(4S)-5-amino-4-[5-[4-[(R)-[2-chloro-3-(1-methylazetidin-3-yl)oxy-phenyl]-phenyl-methyl]piperidine-1-carbonyl]-6-methyl-1-oxo-isoindolin-2-yl]-5-oxo-pentanoate as an off-white foam (43.2 mg, 76% p., 20% y.). m/z [M+H]⁺=729.4.

### Example 42

In a sealed vial, a solution of tert-butyl(4S)-5-amino-4-[5-[4-[(R)-[2-chloro-3-(1-methylazetidin-3-yl)oxy-phenyl]-phenyl-methyl]piperidine-1-carbonyl]-6-methyl-1-oxo-isoindolin-2-yl]-5-oxo-pentanoate (40.0 mg, 0.0548 mmol) and 4-methylbenzenesulfonic acid hydrate (31.3 mg, 0.165 mmol) in acetonitrile (410 μL) was stirred at 85° C. for 3 h. The mixture was cooled to ambient temperature then EtOAc and water were added. The mixture was poured into a saturated aqueous solution of NaHCO₃ and extracted with EtOAc (2×). The combined organic layers were washed with brine, filtered through hydrophobic paper, and concentrated. The crude was purified by reverse-phase preparative chromatography using a gradient of acetonitrile in water from 0% to 100% (0.1% AcOH). The desired fractions were combined, and acetonitrile evaporated. A saturated aqueous solution of NaHCO₃ was added and the mixture was extracted with EtOAc (2×). The combined organic layers were washed with brine, filtered through hydrophobic paper, and concentrated. The residue was solubilized in DCM and added dropwise to stirred n-pentane. The obtained suspension was stirred at ambient temperature for 2 h and filtered. The residue was washed with n-pentane and dried under reduced pressure at 50° C. for 16 h to afford (3S)-3-[5-[4-[(R)-[2-chloro-3-(1-methylazetidin-3-yl)oxy-phenyl]-phenyl-methyl]piperidine-1-carbonyl]-6-methyl-1-oxo-isoindolin-2-yl]piperidine-2,6-dione as a white solid (14.0 mg, 100% p., 39% y.). m/z [M+H]⁺=655.4, ¹H NMR (600 MHz, DMSO-d₆): δ (ppm) 10.97 (s, 1H), 7.65-7.58 (m, 1H), 7.52-7.38 (m, 1H), 7.38-7.32 (m, 2H), 7.32-7.26 (m, 2H), 7.26-7.23 (m, 1H), 7.23-7.20 (m, 1H), 7.19-7.10 (m, 1H), 6.79-6.61 (m, 1H), 5.22-5.00 (m, 1H), 4.80-4.66 (m, 1H), 4.51 (br d, J=1.0 Hz, 1H), 4.41 (br d, J=9.4 Hz, 1H), 4.35-4.25 (m, 1H), 4.19 (br d, J=11.3 Hz, 1H), 3.81-3.63 (m, 2H), 3.26-3.14 (m, 1H), 2.94 (br dd, J=14.6, 7.1 Hz, 4H), 2.83-2.68 (m, 1H), 2.65-2.52 (m, 2H), 2.45-2.35 (m, 1H), 2.26 (d, J=15.7 Hz, 6H), 2.05-1.91 (m, 1H), 1.70-0.87 (m, 4H), [α]_{D}=+19°·mL·dm⁻¹.g⁻¹[20° C., Na lamp, 589 nm, C=1.2 mg/mL, DMSO].

### Example 43

### (3S)-3-[5-[4-[(S)-[2-chloro-3-[(1-methylimidazol-2-yl)methoxy]phenyl]-phenyl-methyl]piperidine-1-carbonyl]-6-methyl-1-oxo-isoindolin-2-yl]piperidine-2,6-dione hydrochloride

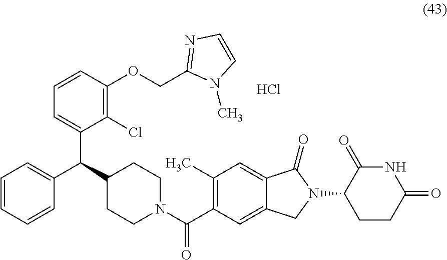 [SMILES: Cc1cc2c(cc1C(=O)N1CCC([C@@H](c3ccccc3)c3cccc(OCc4nccn4C)c3Cl)CC1)CN([C@H]1CCC(=O)NC1=O)C2=O ; Cl]

### Intermediate 43A: tert-butyl(4S)-5-amino-4-[5-[4-[(S)-[2-chloro-3-[(1-methylimidazol-2-yl)methoxy]phenyl]-phenyl-methyl]piperidine-1-carbonyl]-6-methyl-1-oxo-isoindolin-2-yl]-5-oxo-pentanoate

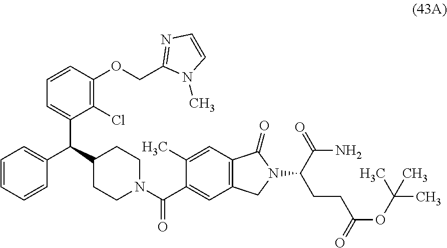 [SMILES: Cc1cc2c(cc1C(=O)N1CCC([C@@H](c3ccccc3)c3cccc(OCc4nccn4C)c3Cl)CC1)CN([C@@H](CCC(=O)OC(C)(C)C)C(N)=O)C2=O]

To a stirred solution of tert-butyl(4S)-5-amino-4-[5-[4-[(S)-(2-chloro-3-hydroxy-phenyl)-phenyl-methyl]piperidine-1-carbonyl]-6-methyl-1-oxo-isoindolin-2-yl]-5-oxo-pentanoate (143 mg, 0.216 mmol) in dry THF (1.4 mL) were added successively triphenylphosphane (170 mg, 0.649 mmol), (1-methyl-1H-imidazol-2-yl)methanol (75.0 mg, 0.649 mmol), and then DIAD (128 μL, 0.649 mmol). The reaction mixture was stirred at ambient temperature for 72 h. Additional (1-methyl-1H-imidazol-2-yl)methanol (50.0 mg, 0.433 mmol) and DIAD (85.2 μL, 0.433 mmol) were added and the reaction mixture was stirred at 50° C. overnight. The reaction mixture was concentrated under reduced pressure. The crude was purified by flash chromatography on silica gel using a gradient of EtOAc/EtOH (3/1) (0.5% conc. NH₄OH) in n-heptane from 5% to 100% to afford tert-butyl(4S)-5-amino-4-[5-[4-[(S)-[2-chloro-3-[(1-methylimidazol-2-yl) methoxy]phenyl]-phenyl-methyl]piperidine-1-carbonyl]-6-methyl-1-oxo-isoindolin-2-yl]-5-oxo-pentanoate as a yellow foam (156 mg, 53% p., 51% y.). m/z [M+H]⁺=754.3.

### Example 43

To a stirred solution of tert-butyl(4S)-5-amino-4-[5-[4-[(S)-[2-chloro-3-[(1-methylimidazol-2-yl)methoxy]phenyl]-phenyl-methyl]piperidine-1-carbonyl]-6-methyl-1-oxo-isoindolin-2-yl]-5-oxo-pentanoate (156 mg, 0.110 mmol) in acetonitrile (1.1 mL) was added 4-methylbenzenesulfonic acid hydrate (104 mg, 0.548 mmol). The reaction mixture was stirred at 80° C. for 3 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure. The solid was solubilized in 1,4-dioxane (3.0 mL) and a 4 M solution of HCl in 1,4-dioxane (300 μL) was added. The reaction mixture was stirred at ambient temperature for 30 min and Et₂O (30 mL) was added. After 1 h, the precipitate was filtered, washed with Et₂O and dried under reduced pressure overnight to afford (3S)-3-[5-[4-[(S)-[2-chloro-3-[(1-methylimidazol-2-yl)methoxy]phenyl]-phenyl-methyl]piperidine-1-carbonyl]-6-methyl-1-oxo-isoindolin-2-yl]piperidine-2,6-dione hydrochloride as a white powder (13.8 mg, 97.4% p., 17% y.). m/z [M+H]⁺=680.3. ¹H NMR (500 MHz, DMSO-d₆): δ (ppm) 11.29-10.36 (m, 1H), 7.74-7.68 (m, 1H), 7.66-7.56 (m, 2H), 7.50-7.11 (m, 9H), 5.52-5.30 (m, 2H), 5.17-5.03 (m, 1H), 4.63-4.07 (m, 4H), 3.94-3.74 (m, 3H), 3.22-2.56 (m, 6H), 2.45-2.20 (m, 4H), 2.07-1.94 (m, 1H), 1.64-1.05 (m, 4H), [α]_{D}=−15°·mL·dm⁻¹.g⁻¹[20° C., Na lamp, 589 nm, C=1.0 mg/mL, DMSO].

### Example 44

### (3S)-3-[5-[4-[(S)-[2-chloro-3-[(1-methylimidazol-4-yl)methoxy]phenyl]-phenyl-methyl]piperidine-1-carbonyl]-6-methyl-1-oxo-isoindolin-2-yl]piperidine-2,6-dione hydrochloride

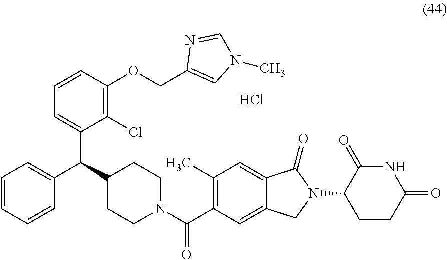 [SMILES: Cc1cc2c(cc1C(=O)N1CCC([C@@H](c3ccccc3)c3cccc(OCc4cn(C)cn4)c3Cl)CC1)CN([C@H]1CCC(=O)NC1=O)C2=O ; Cl]

### Intermediate 44A: tert-butyl(4S)-5-amino-4-[5-[4-[(S)-[2-chloro-3-[(1-methylimidazol-4-yl)methoxy]phenyl]-phenyl-methyl]piperidine-1-carbonyl]-6-methyl-1-oxo-isoindolin-2-yl]-5-oxo-pentanoate

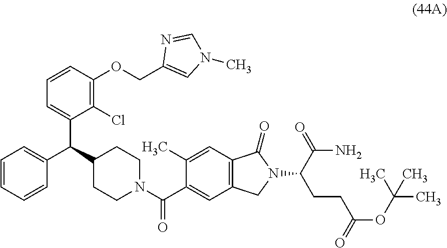 [SMILES: Cc1cc2c(cc1C(=O)N1CCC([C@@H](c3ccccc3)c3cccc(OCc4cn(C)cn4)c3Cl)CC1)CN([C@@H](CCC(=O)OC(C)(C)C)C(N)=O)C2=O]

To a stirred solution of tert-butyl(4S)-5-amino-4-[5-[4-[(S)-(2-chloro-3-hydroxy-phenyl)-phenyl-methyl]piperidine-1-carbonyl]-6-methyl-1-oxo-isoindolin-2-yl]-5-oxo-pentanoate (143 mg, 0.216 mmol) in dry THF (1.5 mL) were added successively triphenylphosphane (170 mg, 0.649 mmol), (1-methyl-1H-imidazol-4-yl)methanol (72.8 mg, 0.649 mmol), and then DIAD (128 μL, 0.649 mmol). The reaction mixture was stirred at ambient temperature for 72 h. Additional (1-methyl-1H-imidazol-4-yl)methanol (48.5 mg, 0.433 mmol) and DIAD (85.2 μL, 0.433 mmol) were added and the reaction mixture was stirred at 50° C. overnight. The reaction mixture was concentrated under reduced pressure. The crude was purified by flash chromatography on silica gel using a gradient of EtOAc/EtOH (3/1) (0.5% conc. NH₄OH) in n-heptane from 5% to 100% to afford (tert-butyl(4S)-5-amino-4-[5-[4-[(S)-[2-chloro-3-[(1-methylimidazol-4-yl) methoxy]phenyl]-phenyl-methyl]piperidine-1-carbonyl]-6-methyl-1-oxo-isoindolin-2-yl]-5-oxo-pentanoate as a yellow foam (65.0 mg, 58% p., 23% y.). m/z [M+H]⁺=754.3.

### Example 44

To a stirred solution of tert-butyl(4S)-5-amino-4-[5-[4-[(S)-[2-chloro-3-[(1-methylimidazol-4-yl)methoxy]phenyl]-phenyl-methyl]piperidine-1-carbonyl]-6-methyl-1-oxo-isoindolin-2-yl]-5-oxo-pentanoate (65.0 mg, 0.0500 mmol) in acetonitrile (510 mL) was added 4-methylbenzenesulfonic acid hydrate (47.5 mg, 0.250 mmol). The reaction mixture was stirred at 80° C. for 3 h. The reaction mixture was cooled to ambient temperature. The reaction mixture was quenched with a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure. The solid was solubilized in 1,4-dioxane (1.4 mL) and a 4 M solution of HCl in 1,4-dioxane (140 μL) was added. The reaction mixture was stirred at ambient temperature for 30 min and Et₂O (14 mL) was added. After 1 h, the precipitate was filtered, washed with Et₂O, and dried under reduced pressure overnight to afford (3S)-3-[5-[4-[(S)-[2-chloro-3-[(1-methylimidazol-4-yl) methoxy]phenyl]-phenyl-methyl]piperidine-1-carbonyl]-6-methyl-1-oxo-isoindolin-2-yl]piperidine-2,6-dione hydrochloride as a white powder (6.15 mg, 90.0% p., 15% y.). m/z [M+H]⁺=680.3, ¹H NMR (600 MHz, DMSO-d₆): δ (ppm) 11.12-10.87 (m, 1H), 9.03-8.80 (m, 1H), 7.82-7.69 (m, 1H), 7.63-7.58 (m, 1H), 7.49-7.06 (m, 9H), 5.06 (br d, J=4.8 Hz, 3H), 4.58-4.12 (m, 4H), 3.88-3.74 (m, 3H), 3.25-2.56 (m, 6H), 2.46-2.17 (m, 4H), 2.06-1.92 (m, 1H), 1.56-1.02 (m, 4H), [α]_{D}=−38°·mL·dm⁻¹ g⁻¹[20° C., Na lamp, 589 nm, C=0.7 mg/mL, DMSO].

### Example 45

### (3S)-3-[5-[4-[(R)-[2-chloro-3-[2-(methylamino) ethoxy]phenyl]-phenyl-methyl]piperidine-1-carbonyl]-6-methyl-1-oxo-isoindolin-2-yl]piperidine-2,6-dione hydrochloride

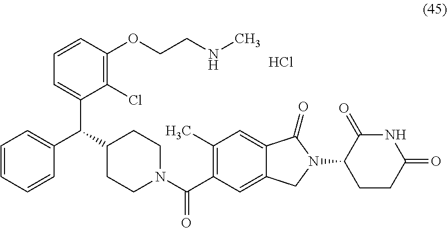 [SMILES: CNCCOc1cccc([C@@H](c2ccccc2)C2CCN(C(=O)c3cc4c(cc3C)C(=O)N([C@H]3CCC(=O)NC3=O)C4)CC2)c1Cl ; Cl]

### Intermediate 45A: tert-butyl(4S)-5-amino-4-[5-[4-[(R)-[3-[2-[tert-butoxycarbonyl(methyl)amino]ethoxy]-2-chloro-phenyl]-phenyl-methyl]piperidine-1-carbonyl]-6-methyl-1-oxo-isoindolin-2-yl]-5-oxo-pentanoate

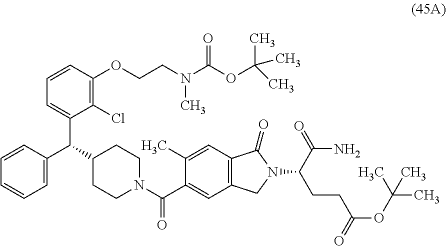 [SMILES: Cc1cc2c(cc1C(=O)N1CCC([C@H](c3ccccc3)c3cccc(OCCN(C)C(=O)OC(C)(C)C)c3Cl)CC1)CN([C@@H](CCC(=O)OC(C)(C)C)C(N)=O)C2=O]

To a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.227 mmol) in dry THF (1.5 mL) were added successively triphenylphosphane (179 mg, 0.682 mmol), tert-butyl(2-hydroxyethyl)methylcarbamate (116 μL, 0.682 mmol), and then DIAD (134 μL, 0.682 mmol). The reaction mixture was stirred at ambient temperature for 48 h. The reaction mixture was concentrated under reduced pressure. The crude was purified by flash chromatography on silica gel using a gradient of EtOAc/EtOH (3/1) (0.5% conc. NH₄OH) in n-heptane from 5% to 100% to afford tert-butyl(4S)-5-amino-4-[5-[4-[(R)-[3-[2-[tert-butoxycarbonyl(methyl)amino]ethoxy]-2-chloro-phenyl]-phenyl-methyl]piperidine-1-carbonyl]-6-methyl-1-oxo-isoindolin-2-yl]-5-oxo-pentanoate as a white solid (130 mg, 77% p., 59% y.). m/z [M-Boc+2H]⁺=717.4, ¹H NMR (400 MHz, DMSO-d₆): δ (ppm) 7.67-7.51 (m, 4H), 7.49-7.08 (m, 9H), 6.96 (dd, J=23.9, 6.7 Hz, 1H), 4.71 (dd, J=10.5, 3.8 Hz, 1H), 4.54 (d, J=17.8 Hz, 2H), 4.41 (d, J=17.7 Hz, 1H), 4.20 (t, J=8.9 Hz, 1H), 4.03 (q, J=9.8 Hz, 2H), 3.58-3.42 (m, 2H), 3.19 (t, J=6.2 Hz, 1H), 3.09-2.72 (m, 5H), 2.56 (d, J=11.5 Hz, 1H), 2.32 (d, J=5.8 Hz, 1H), 2.23 (s, 2H), 2.14 (t, J=4.1 Hz, 3H), 1.62 (s, 1H), 1.42-1.23 (m, 18H), 1.18-0.88 (m, 2H).

### Example 45

To a stirred solution of tert-butyl(4S)-5-amino-4-[5-[4-[(R)-[3-[2-[tert-butoxycarbonyl(methyl)amino]ethoxy]-2-chloro-phenyl]-phenyl-methyl]piperidine-1-carbonyl]-6-methyl-1-oxo-isoindolin-2-yl]-5-oxo-pentanoate (142 mg, 0.134 mmol) in acetonitrile (1.0 mL) at ambient temperature was added 4-methylbenzenesulfonic acid hydrate (127 mg, 0.668 mmol). The reaction mixture was stirred at 80° C. for 2 h. The reaction was quenched with the addition of a saturated aqueous solution of NaHCO₃. EtOAc was added to the mixture. The aqueous layer was extracted with EtOAc (2×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). Fractions containing the product were combined and concentrated until most of the acetonitrile was removed. The resulting solution was basified with a saturated aqueous solution of NaHCO₃ and EtOAc was added. The aqueous layer was extracted with EtOAc (2×). The combined organic layers were washed with brine, dried over Na₂SO₄, and concentrated under reduced pressure. The residue was solubilized in a minimum of 1,4-dioxane and a 4 M solution of HCl in 1,4-dioxane (334 μL, 1.34 mmol) was added. The reaction mixture was stirred at ambient temperature for 30 min and then Et₂O was added. The resulting precipitate was stirred for 30 min, filtered, washed with Et₂O, and dried under reduced pressure at 50° C. for 12 h to afford (3S)-3-[5-[4-[(R)-[2-chloro-3-[2-(methylamino) ethoxy]phenyl]-phenyl-methyl]piperidine-1-carbonyl]-6-methyl-1-oxo-isoindolin-2-yl]piperidine-2,6-dione hydrochloride as a white powder (34.9 mg, 99.8% p., 38% y.). m/z [M+H]⁺=643.3, ¹H NMR (500 MHz, DMSO-d₆): δ (ppm) 11.11-10.80 (m, 1H), 8.72 (br dd, J=1.8, 0.9 Hz, 2H), 7.65-7.58 (m, 1H), 7.49-7.10 (m, 8H), 7.06-6.94 (m, 1H), 5.17-5.01 (m, 1H), 4.58-4.14 (m, 6H), 3.42-3.35 (m, 2H), 3.28-3.16 (m, 1H), 3.07-2.71 (m, 3H), 2.70-2.64 (m, 3H), 2.63-2.54 (m, 2H), 2.44-2.22 (m, 4H), 2.06-1.93 (m, 1H), 1.71-0.84 (m, 4H), [a]_{D}=+7°·mL·dm⁻¹ g⁻¹[20° C., Na lamp, 589 nm, C=1.8 mg/mL, DMSO].

### Example 46

### (3S)-3-[5-[4-[(R)-[3-(2-aminoethoxy)-2-chloro-phenyl]-phenyl-methyl]piperidine-1-carbonyl]-6-methyl-1-oxo-isoindolin-2-yl]piperidine-2,6-dione hydrochloride

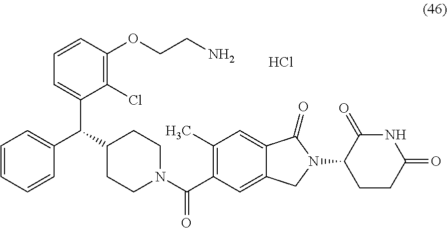 [SMILES: Cc1cc2c(cc1C(=O)N1CCC([C@H](c3ccccc3)c3cccc(OCCN)c3Cl)CC1)CN([C@H]1CCC(=O)NC1=O)C2=O ; Cl]

### Intermediate 46A: tert-butyl(4S)-5-amino-4-[5-[4-[(R)-[3-[2-(tert-butoxycarbonylamino) ethoxy]-2-chloro-phenyl]-phenyl-methyl]piperidine-1-carbonyl]-6-methyl-1-oxo-isoindolin-2-yl]-5-oxo-pentanoate

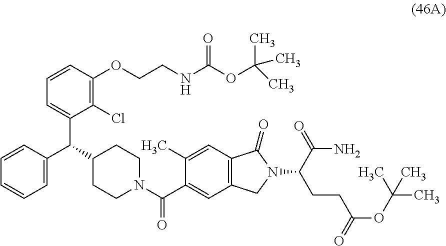 [SMILES: Cc1cc2c(cc1C(=O)N1CCC([C@H](c3ccccc3)c3cccc(OCCNC(=O)OC(C)(C)C)c3Cl)CC1)CN([C@@H](CCC(=O)OC(C)(C)C)C(N)=O)C2=O]

To a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.227 mmol) in dry THF (1.5 mL) were added successively triphenylphosphane (179 mg, 0.682 mmol), tert-butyl(2-hydroxyethyl) carbamate (105 μL, 0.682 mmol), and then DIAD (134 μL, 0.682 mmol). The reaction mixture was stirred at ambient temperature for 48 h. The reaction mixture was concentrated under reduced pressure. The crude was purified by flash chromatography on silica gel using a gradient of EtOAc/EtOH (3/1) (0.5% conc. NH₄OH) in n-heptane from 5% to 100% to afford tert-butyl(4S)-5-amino-4-[5-[4-[(R)-[3-[2-(tert-butoxycarbonylamino) ethoxy]-2-chloro-phenyl]-phenyl-methyl]piperidine-1-carbonyl]-6-methyl-1-oxo-isoindolin-2-yl]-5-oxo-pentanoate as a white solid (131 mg, 71% p., 51% y.). m/z [M+Na]⁺=825.4.[a]_{D}=−34°·mL·dm⁻¹.g⁻¹[20° C., Na lamp, 589 nm, C=1.2 mg/mL, DMSO].

### Example 46

To a stirred solution of tert-butyl(4S)-5-amino-4-[5-[4-[(R)-[3-[2-(tert-butoxycarbonylamino) ethoxy]-2-chloro-phenyl]-phenyl-methyl]piperidine-1-carbonyl]-6-methyl-1-oxo-isoindolin-2-yl]-5-oxo-pentanoate (131 mg, 0.116 mmol) in acetonitrile (1.0 mL) at ambient temperature was added 4-methylbenzenesulfonic acid hydrate (110 mg, 0.579 mmol). The reaction mixture was stirred at 80° C. for 2 h. The reaction was quenched with a saturated aqueous solution of NaHCO₃. EtOAc was added to the mixture. The aqueous layer was extracted with EtOAc (2×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). Fractions containing were combined and concentrated until most of the acetonitrile was removed. The resulting solution was basified with a saturated aqueous solution of NaHCO₃ and EtOAc was added. The aqueous layer was extracted with EtOAc (2×). The combined organic layers were washed with brine, dried over Na₂SO₄, and concentrated under reduced pressure. The residue was solubilized in a minimum of 1,4-dioxane and a 4 M solution of HCl in 1,4-dioxane (334 μL, 1.34 mmol) was added. The reaction mixture was stirred at ambient temperature for 30 min and then Et₂O was added. The resulting precipitate was stirred for 30 min, filtered, washed with Et₂O, and dried under reduced pressure at 50° C. for 12 h to afford (3S)-3-[5-[4-[(R)-[3-(2-aminoethoxy)-2-chloro-phenyl]-phenyl-methyl]piperidine-1-carbonyl]-6-methyl-1-oxo-isoindolin-2-yl]piperidine-2,6-dione hydrochloride as a white powder (47.2 mg, 99.8% p., 61% y.). m/z [M+H]⁺=629.3, 1H NMR (500 MHz, DMSO-d₆): δ (ppm) 11.16-10.83 (m, 1H), 8.15-7.91 (m, 3H), 7.62 (br d, J=7.8 Hz, 1H), 7.51-7.09 (m, 8H), 7.07-6.91 (m, 1H), 5.19-5.02 (m, 1H), 4.65-4.02 (m, 6H), 3.29-3.16 (m, 3H), 3.07-2.67 (m, 3H), 2.65-2.54 (m, 2H), 2.45-2.22 (m, 4H), 2.08-1.93 (m, 1H), 1.68-0.89 (m, 4H), [a]_{D}=+6°·mL·dm⁻¹ g⁻¹[20° C., Na lamp, 589 nm, C=1.8 mg/mL, DMSO].

### Example 47

### (3S)-3-[5-[4-[(R)-[2-chloro-3-[(1-methylimidazol-2-yl)methoxy]phenyl]-phenyl-methyl]piperidine-1-carbonyl]-6-methyl-1-oxo-isoindolin-2-yl]piperidine-2,6-dione

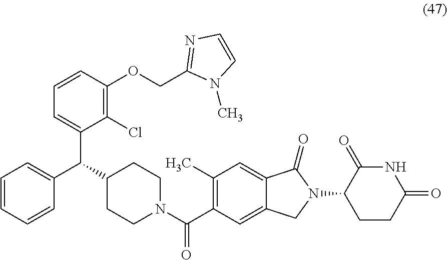 [SMILES: Cc1cc2c(cc1C(=O)N1CCC([C@H](c3ccccc3)c3cccc(OCc4nccn4C)c3Cl)CC1)CN([C@H]1CCC(=O)NC1=O)C2=O]

### Intermediate 47A: tert-butyl(4S)-5-amino-4-[5-[4-[(R)-[2-chloro-3-[(1-methylimidazol-2-yl)methoxy]phenyl]-phenyl-methyl]piperidine-1-carbonyl]-6-methyl-1-oxo-isoindolin-2-yl]-5-oxo-pentanoate

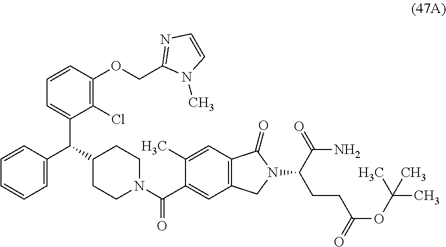 [SMILES: Cc1cc2c(cc1C(=O)N1CCC([C@H](c3ccccc3)c3cccc(OCc4nccn4C)c3Cl)CC1)CN([C@@H](CCC(=O)OC(C)(C)C)C(N)=O)C2=O]

To a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.227 mmol) in dry THF (1.5 mL) were added successively triphenylphosphane (179 mg, 0.682 mmol), (1-methyl-1H-imidazol-2-yl)methanol (76.4 mg, 0.682 mmol), and then DIAD (134 μL, 0.682 mmol). The reaction mixture was stirred at ambient temperature for 72 h. The reaction mixture was concentrated under reduced pressure. The crude was purified by flash chromatography on silica gel using a gradient of EtOAc/EtOH (3/1) (0.5% conc. NH₄OH) in n-heptane from 5% to 100% to afford tert-butyl(4S)-5-amino-4-[5-[4-[(R)-[2-chloro-3-[(1-methylimidazol-2-yl) methoxy]phenyl]-phenyl-methyl]piperidine-1-carbonyl]-6-methyl-1-oxo-isoindolin-2-yl]-5-oxo-pentanoate as a white solid (68.0 mg, 64% p., 25% y.).

### Example 47

To a stirred solution of tert-butyl(4S)-5-amino-4-[5-[4-[(R)-[2-chloro-3-[(1-methylimidazol-2-yl)methoxy]phenyl]-phenyl-methyl]piperidine-1-carbonyl]-6-methyl-1-oxo-isoindolin-2-yl]-5-oxo-pentanoate (68.0 mg, 0.0577 mmol) in acetonitrile (1.0 mL) at ambient temperature was added 4-methylbenzenesulfonic acid hydrate (32.9 mg, 0.173 mmol). The reaction mixture was stirred at 80° C. for 3 h. The reaction was quenched with a saturated aqueous solution of NaHCO₃. EtOAc was added to the mixture. The aqueous layer was extracted with EtOAc (2×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). Fractions containing were combined and concentrated until most of the acetonitrile was removed. The resulting solution was basified with a saturated aqueous solution of NaHCO₃ and EtOAc was added. The aqueous layer was extracted with EtOAc (2×). The combined organic layers were washed with brine, dried over Na₂SO₄ and concentrated. The residue was solubilized in a minimum of 1,4-dioxane and a 4 M solution of HCl in 1,4-dioxane (334 μL, 1.34 mmol) was added. The reaction mixture was stirred at ambient temperature for 30 min and Et₂O was added. The resulting precipitate was stirred for 30 min, filtered, washed with Et₂O, and dried under reduced pressure at 50° C. for 12 h to afford (3S)-3-[5-[4-[(R)-[2-chloro-3-[(1-methylimidazol-2-yl)methoxy]phenyl]-phenyl-methyl]piperidine-1-carbonyl]-6-methyl-1-oxo-isoindolin-2-yl]piperidine-2,6-dione as a white powder (11.0 mg, 99.8% p., 28% y.). m/z [M+H]⁺=680.4, ¹H NMR (600 MHz, DMSO-d₆): δ (ppm) 11.03-10.92 (m, 1H), 7.75-7.49 (m, 3H), 7.47-7.21 (m, 7H), 7.21-7.10 (m, 2H), 5.47-5.33 (m, 2H), 5.18-5.04 (m, 1H), 4.60-4.46 (m, 1H), 4.45-4.35 (m, 1H), 4.33-4.23 (m, 1H), 4.23-4.14 (m, 1H), 3.89-3.80 (m, 3H), 3.27-3.15 (m, 1H), 3.05-2.94 (m, 1H), 2.94-2.84 (m, 1H), 2.83-2.68 (m, 1H), 2.63-2.54 (m, 2H), 2.42-2.35 (m, 1H), 2.35-2.21 (m, 3H), 2.04-1.92 (m, 1H), 1.69-0.85 (m, 4H), [a]_{D}=−8°·mL·dm⁻¹ g⁻¹[20° C., Na lamp, 589 nm, C=0.9 mg/mL, DMSO].

### Example 48

### (3S)-3-[5-[4-[(R)-[2-chloro-3-[(1-methylimidazol-4-yl)methoxy]phenyl]-phenyl-methyl]piperidine-1-carbonyl]-6-methyl-1-oxo-isoindolin-2-yl]piperidine-2,6-dione

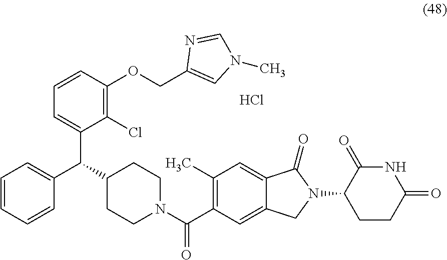 [SMILES: Cc1cc2c(cc1C(=O)N1CCC([C@H](c3ccccc3)c3cccc(OCc4cn(C)cn4)c3Cl)CC1)CN([C@H]1CCC(=O)NC1=O)C2=O ; Cl]

### Intermediate 48A: tert-butyl(4S)-5-amino-4-[5-[4-[(R)-[2-chloro-3-[(1-methylimidazol-4-yl)methoxy]phenyl]-phenyl-methyl]piperidine-1-carbonyl]-6-methyl-1-oxo-isoindolin-2-yl]-5-oxo-pentanoate

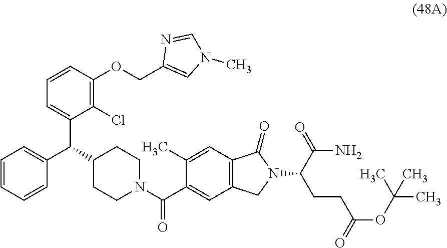 [SMILES: Cc1cc2c(cc1C(=O)N1CCC([C@H](c3ccccc3)c3cccc(OCc4cn(C)cn4)c3Cl)CC1)CN([C@@H](CCC(=O)OC(C)(C)C)C(N)=O)C2=O]

To a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.227 mmol) in dry THF (1.5 mL) were added successively triphenylphosphane (179 mg, 0.682 mmol), (1-methyl-1H-imidazol-2-yl)methanol (76.4 mg, 0.682 mmol), and then DIAD (134 μL, 0.682 mmol). The reaction mixture was stirred at ambient temperature for 48 h. The reaction mixture was concentrated under reduced pressure. The crude was purified by flash chromatography on silica gel using a gradient of EtOAc/EtOH (3/1) (0.5% conc. NH₄OH) in n-heptane from 5% to 100% to afford tert-butyl(4S)-5-amino-4-[5-[4-[(R)-[2-chloro-3-[(1-methylimidazol-4-yl) methoxy]phenyl]-phenyl-methyl]piperidine-1-carbonyl]-6-methyl-1-oxo-isoindolin-2-yl]-5-oxo-pentanoate as a white solid (27.0 mg, 47% p., 7% y.). m/z [M+H]⁺=754.4.

### Example 48

To a stirred solution of tert-butyl(4S)-5-amino-4-[5-[4-[(R)-[2-chloro-3-[(1-methylimidazol-4-yl)methoxy]phenyl]-phenyl-methyl]piperidine-1-carbonyl]-6-methyl-1-oxo-isoindolin-2-yl]-5-oxo-pentanoate (27.0 mg, 0.0168 mmol) in acetonitrile (1.0 mL) at ambient temperature was added 4-methylbenzenesulfonic acid hydrate (9.6 mg, 0.0505 mmol). The reaction mixture was stirred at 80° C. for 3 h. The reaction was quenched with the addition of a saturated aqueous solution of NaHCO₃. EtOAc was added to the mixture. The aqueous layer was extracted with EtOAc (2×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). Fractions containing were combined and concentrated until most of the acetonitrile was removed. The resulting solution was basified with a saturated aqueous solution of NaHCO₃ and EtOAc was added. The aqueous layer was extracted with EtOAc (2×). The combined organic layers were washed with brine, dried over Na₂SO₄ and concentrated. The residue was solubilized in a minimum of 1,4-dioxane and a 4 M solution of HCl in 1,4-dioxane (21.0 μL, 0.0841 mmol) was added. The reaction mixture was stirred at ambient temperature for 30 min and then Et₂O was added. The resulting precipitate was stirred for 30 min, filtered, washed with Et₂O, and dried under reduced pressure at 50° C. for 12 h to afford (3S)-3-[5-[4-[(R)-[2-chloro-3-[(1-methylimidazol-4-yl)methoxy]phenyl]-phenyl-methyl]piperidine-1-carbonyl]-6-methyl-1-oxo-isoindolin-2-yl]piperidine-2,6-dione hydrochloride as a white powder (6.0 mg, 97.4% p., 48% y.). m/z [M+H]⁺=680.4. ¹H NMR (600 MHz, DMSO-d₆): δ (ppm) 11.18-10.74 (m, 1H), 9.09-8.49 (m, 1H), 7.74-7.64 (m, 1H), 7.64-7.58 (m, 1H), 7.48-7.04 (m, 9H), 5.23-5.02 (m, 3H), 4.58-4.46 (m, 1H), 4.45-4.35 (m, 1H), 4.34-4.23 (m, 1H), 4.23-4.15 (m, 1H), 3.85-3.77 (m, 3H), 3.27-3.15 (m, 1H), 3.04-2.94 (m, 1H), 2.94-2.84 (m, 1H), 2.82-2.69 (m, 1H), 2.63-2.53 (m, 2H), 2.43-2.35 (m, 1H), 2.35-2.22 (m, 3H), 2.04-1.92 (m, 1H), 1.69-0.83 (m, 4H).

### Example 49

### (3S)-3-[5-[4-[(R)-[2-chloro-3-[(2-methyl-2-azaspiro[3.3]heptan-6-yl)oxy]phenyl]-phenyl-methyl]piperidine-1-carbonyl]-6-methyl-1-oxo-isoindolin-2-yl]piperidine-2,6-dione

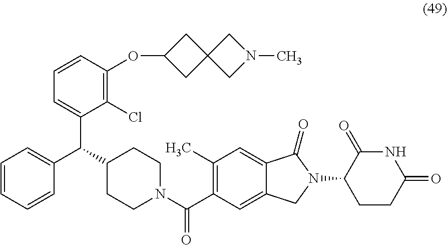 [SMILES: Cc1cc2c(cc1C(=O)N1CCC([C@H](c3ccccc3)c3cccc(OC4CC5(C4)CN(C)C5)c3Cl)CC1)CN([C@H]1CCC(=O)NC1=O)C2=O]

### Intermediate 49A: tert-butyl(4S)-5-amino-4-[5-[4-[(R)-[2-chloro-3-[(2-methyl-2-azaspiro[3.3]heptan-6-yl)oxy]phenyl]-phenyl-methyl]piperidine-1-carbonyl]-6-methyl-1-oxo-isoindolin-2-yl]-5-oxo-pentanoate

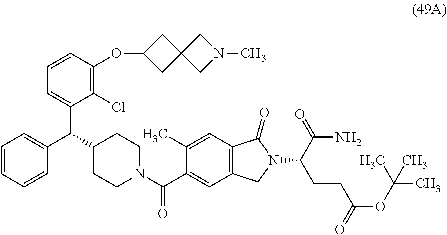 [SMILES: Cc1cc2c(cc1C(=O)N1CCC([C@H](c3ccccc3)c3cccc(OC4CC5(C4)CN(C)C5)c3Cl)CC1)CN([C@@H](CCC(=O)OC(C)(C)C)C(N)=O)C2=O]

To a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.211 mmol) in dry THF (1.4 mL) were added successively triphenylphosphane (166 mg, 0.634 mmol), 2-methyl-2-azaspiro[3.3]heptan-6-ol (84.9 mg, 0.634 mmol), and then DIAD (125 μL, 0.634 mmol). The reaction mixture was stirred at ambient temperature for 72 h. The reaction mixture was concentrated under reduced pressure. The crude was purified by flash chromatography on silica gel using a gradient of EtOAc/EtOH (3/1) (0.5% conc. NH₄OH) in n-heptane from 5% to 100% to afford tert-butyl(4S)-5-amino-4-[5-[4-[(R)-[2-chloro-3-[(2-methyl-2-azaspiro[3.3]heptan-6-yl)oxy]phenyl]-phenyl-methyl]piperidine-1-carbonyl]-6-methyl-1-oxo-isoindolin-2-yl]-5-oxo-pentanoate as a white foam (58.0 mg, 73% p., 26% y.). m/z [M+H]⁺=769.5.

### Example 49

To a stirred solution of tert-butyl(4S)-5-amino-4-[5-[4-[(R)-[2-chloro-3-[(2-methyl-2-azaspiro[3.3]heptan-6-yl)oxy]phenyl]-phenyl-methyl]piperidine-1-carbonyl]-6-methyl-1-oxo-isoindolin-2-yl]-5-oxo-pentanoate (54.0 mg, 0.0702 mmol) in acetonitrile (700 μL) was added 4-methylbenzenesulfonic acid hydrate (66.8 mg, 0.351 mmol). The reaction mixture was stirred at 80° C. for 2 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). Acetonitrile was removed under reduced pressure. The aqueous layer was basified with a saturated aqueous solution of NaHCO₃. The precipitate was triturated for 1 h, filtered, washed with water, and dried under reduced pressure at 50° C. overnight to afford (3S)-3-[5-[4-[(R)-[2-chloro-3-[(2-methyl-2-azaspiro[3.3]heptan-6-yl)oxy]phenyl]-phenyl-methyl]piperidine-1-carbonyl]-6-methyl-1-oxo-isoindolin-2-yl]piperidine-2,6-dione as an off-white solid (13.2 mg, 85.3% p., 23% y.). m/z [M+H]⁺=695.3, ¹H NMR (600 MHz, DMSO-d₆): δ (ppm) 10.97 (s, 1H), 7.64-7.50 (m, 1H), 7.45-7.20 (m, 7H), 6.80-6.66 (m, 1H), 5.14-5.02 (m, 1H), 4.62-4.25 (m, 4H), 4.22-4.15 (m, 1H), 3.23-3.09 (m, 5H), 3.00-2.84 (m, 2H), 2.81-2.69 (m, 1H), 2.66-2.52 (m, 4H), 2.38-2.22 (m, 4H), 2.21-2.14 (m, 4H), 2.13-2.05 (m, 2H), 1.98 (br s, 1H), 1.58-0.85 (m, 4H), [α]_{D}=+25°·mL·dm⁻¹ g⁻¹[20° C., Na lamp, 589 nm, C=1.5 mg/mL, DMSO].

### Example 50

### (3S)-3-[5-[4-[(R)-[2-chloro-3-[(3S)-1-(2,2,2-trifluoroethyl)pyrrolidin-3-yl]oxy-phenyl]-phenyl-methyl]piperidine-1-carbonyl]-6-methyl-1-oxo-isoindolin-2-yl]piperidine-2,6-dione

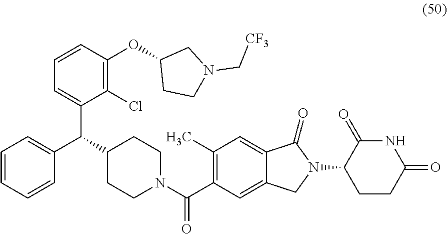 [SMILES: Cc1cc2c(cc1C(=O)N1CCC([C@H](c3ccccc3)c3cccc(O[C@H]4CCN(CC(F)(F)F)C4)c3Cl)CC1)CN([C@H]1CCC(=O)NC1=O)C2=O]

Intermediate 50A: tert-butyl(4S)-5-amino-4-[5-[4-[(R)-[2-chloro-3-[(3S)-1-(2,2,2-trifluoroethyl)pyrrolidin-3-yl]oxy-phenyl]-phenyl-methyl]piperidine-1-carbonyl]-6-methyl-1-oxo-isoindolin-2-yl]-5-oxo-pentanoate

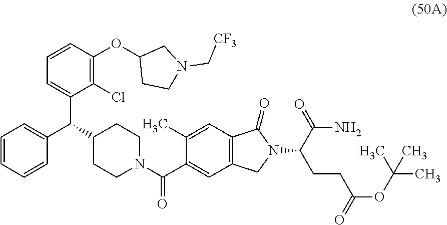 [SMILES: Cc1cc2c(cc1C(=O)N1CCC([C@H](c3ccccc3)c3cccc(OC4CCN(CC(F)(F)F)C4)c3Cl)CC1)CN([C@@H](CCC(=O)OC(C)(C)C)C(N)=O)C2=O]

To a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.227 mmol) in dry THF (1.6 mL) were added successively triphenylphosphane (179 mg, 0.682 mmol), (3R)-1-(2,2,2-trifluoroethyl)pyrrolidin-3-ol (121 mg, 0.682 mmol), and then DIAD (134 μL, 0.682 mmol). The reaction mixture was stirred at ambient temperature overnight. The reaction mixture was concentrated under reduced pressure. The crude was purified by flash chromatography on silica gel using a gradient of EtOAc/EtOH (3/1) (0.5% conc. NH₄OH) in n-heptane from 5% to 100% to afford (tert-butyl(4S)-5-amino-4-[5-[4-[(R)-[2-chloro-3-[(3S)-1-(2,2,2-trifluoroethyl) pyrrolidin-3-yl]oxy-phenyl]-phenyl-methyl]piperidine-1-carbonyl]-6-methyl-1-oxo-isoindolin-2-yl]-5-oxo-pentanoate as a white solid (134 mg, 91% p., 66% y.)), m/z [M+H]⁺=811.3. [α]_{D}=+26°·mL·dm⁻¹ g⁻¹[20° C., Na lamp, 589 nm, C=1.4 mg/mL, MeOH].

### Example 50

To a stirred solution of tert-butyl(4S)-5-amino-4-[5-[4-[(R)-[2-chloro-3-[(3S)-1-(2,2,2-trifluoroethyl)pyrrolidin-3-yl]oxy-phenyl]-phenyl-methyl]piperidine-1-carbonyl]-6-methyl-1-oxo-isoindolin-2-yl]-5-oxo-pentanoate (148 mg, 0.166 mmol) in acetonitrile (1.7 mL) was added 4-methylbenzenesulfonic acid hydrate (157 mg, 0.828 mmol). The reaction mixture was stirred at 80° C. for 4 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). Acetonitrile was removed under reduced pressure. The aqueous layer was basified with a saturated aqueous solution of NaHCO₃. The precipitate was triturated for 1 h, filtered, washed with water, and dried under reduced pressure at 50° C. overnight to afford (3S)-3-[5-[4-[(R)-[2-chloro-3-[(3S)-1-(2,2,2-trifluoroethyl)pyrrolidin-3-yl]oxy-phenyl]-phenyl-methyl]piperidine-1-carbonyl]-6-methyl-1-oxo-isoindolin-2-yl]piperidine-2,6-dione as a white solid (59.7 mg, 98.7% p., 48% y.). m/z [M+H]⁺=737.3, ¹H NMR (600 MHz, DMSO-d₆): δ (ppm) 11.25-10.62 (m, 1H), 7.64-7.54 (m, 1H), 7.50-7.20 (m, 7H), 7.19-7.09 (m, 1H), 6.92-6.80 (m, 1H), 5.20-4.98 (m, 1H), 4.93-4.81 (m, 1H), 4.59-4.14 (m, 4H), 3.29-3.07 (m, 4H), 3.03-2.52 (m, 8H), 2.43-2.15 (m, 5H), 2.04-1.92 (m, 1H), 1.85-1.71 (m, 1H), 1.67-0.87 (m, 4H), [α]_{D}=+21°·mL·dm⁻¹ g⁻¹[20° C., Na lamp, 589 nm, C=1.1 mg/mL, DMSO].

### Example 51

### (3S)-3-[5-[4-[(R)-(2-chloro-3-isopropoxy-phenyl)-phenyl-methyl]piperidine-1-carbonyl]-6-methyl-1-oxo-isoindolin-2-yl]piperidine-2,6-dione

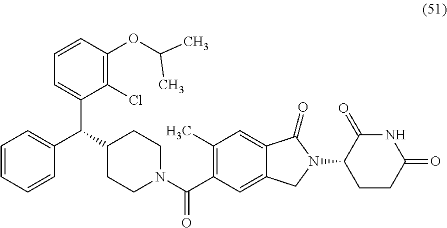 [SMILES: Cc1cc2c(cc1C(=O)N1CCC([C@H](c3ccccc3)c3cccc(OC(C)C)c3Cl)CC1)CN([C@H]1CCC(=O)NC1=O)C2=O]

### Intermediate 51A: tert-butyl(4S)-5-amino-4-[5-[4-[(R)-(2-chloro-3-isopropoxy-phenyl)-phenyl-methyl]piperidine-1-carbonyl]-6-methyl-1-oxo-isoindolin-2-yl]-5-oxo-pentanoate

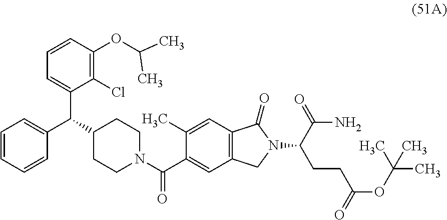 [SMILES: Cc1cc2c(cc1C(=O)N1CCC([C@H](c3ccccc3)c3cccc(OC(C)C)c3Cl)CC1)CN([C@@H](CCC(=O)OC(C)(C)C)C(N)=O)C2=O]

To a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.211 mmol) in dry THF (1.4 mL) were added successively triphenylphosphane (166 mg, 0.634 mmol), propan-2-ol (48.5 μL, 0.634 mmol), and then DIAD (125 μL, 0.634 mmol). The reaction mixture was stirred at ambient temperature for 16 h. The reaction mixture was concentrated under reduced pressure. The crude was purified by flash chromatography on silica gel using a gradient of EtOAc/EtOH (3/1) (0.5% conc. NH₄OH) in n-heptane from 5% to 100% to afford tert-butyl(4S)-5-amino-4-[5-[4-[(R)-(2-chloro-3-isopropoxy-phenyl)-phenyl-methyl]piperidine-1-carbonyl]-6-methyl-1-oxo-isoindolin-2-yl]-5-oxo-pentanoate as a white solid (111 mg, 93% p., 70% y.). m/z [M+H]⁺=702.5. [α]_{D}=+14°·mL·dm⁻¹ g⁻¹[20° C., Na lamp, 589 nm, C=1.8 mg/mL, MeOH].

### Example 51

In a sealed vial, to a solution of tert-butyl(4S)-5-amino-4-[5-[4-[(R)-(2-chloro-3-isopropoxy-phenyl)-phenyl-methyl]piperidine-1-carbonyl]-6-methyl-1-oxo-isoindolin-2-yl]-5-oxo-pentanoate (102 mg, 0.146 mmol) in acetonitrile (2.5 mL) was added 4-methylbenzenesulfonic acid hydrate (83.1 mg, 0.437 mmol). The solution was stirred at 90° C. for 3 h. The mixture was diluted with EtOAc and washed with a saturated aqueous solution of NaHCO₃ and brine. The organic layer was dried over Na₂SO₄, filtered, and concentrated under reduced pressure. The crude was purified by reverse-phase preparative chromatography using a gradient of acetonitrile in water from 0% to 100% (0.1% AcOH). The desired fractions were combined and concentrated under reduced pressure until evaporation of most of the acetonitrile. A saturated aqueous solution of NaHCO₃ was added. The resulting suspension was filtered and dried under at 50° C. under reduced pressure overnight to afford (3S)-3-[5-[4-[(R)-(2-chloro-3-isopropoxy-phenyl)-phenyl-methyl]piperidine-1-carbonyl]-6-methyl-1-oxo-isoindolin-2-yl]piperidine-2,6-dione as a white powder (43.9 mg, 97.8% p., 47% y.). m/z [M+H]⁺=628.3, ¹H NMR (500 MHz, DMSO-d₆): δ (ppm) 11.25-10.71 (m, 1H), 7.64-7.57 (m, 1H), 7.53-7.08 (m, 8H), 7.03-6.87 (m, 1H), 5.15-5.01 (m, 1H), 4.70-4.12 (m, 5H), 3.28-2.53 (m, 6H), 2.44-2.19 (m, 4H), 2.06-1.92 (m, 1H), 1.70-0.89 (m, 10H), [α]_{D}=+11°·mL·dm⁻¹.g⁻¹[20° C., Na lamp, 589 nm, C=1.1 mg/mL, DMSO].

### Example 52

### (3S)-3-[5-[4-[(R)-[2-chloro-3-[(3R)-1-(2,2,2-trifluoroethyl)pyrrolidin-3-yl]oxy-phenyl]-phenyl-methyl]piperidine-1-carbonyl]-6-methyl-1-oxo-isoindolin-2-yl]piperidine-2,6-dione

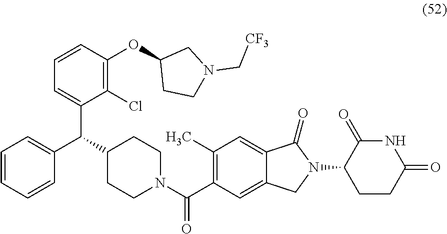 [SMILES: Cc1cc2c(cc1C(=O)N1CCC([C@H](c3ccccc3)c3cccc(O[C@@H]4CCN(CC(F)(F)F)C4)c3Cl)CC1)CN([C@H]1CCC(=O)NC1=O)C2=O]

### Intermediate 52A: tert-butyl(4S)-5-amino-4-[5-[4-[(R)-[2-chloro-3-[(3R)-1-(2,2,2-trifluoroethyl)pyrrolidin-3-yl]oxy-phenyl]-phenyl-methyl]piperidine-1-carbonyl]-6-methyl-1-oxo-isoindolin-2-yl]-5-oxo-pentanoate

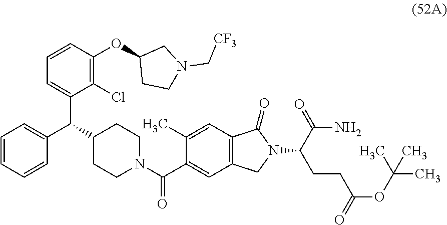 [SMILES: Cc1cc2c(cc1C(=O)N1CCC([C@H](c3ccccc3)c3cccc(O[C@@H]4CCN(CC(F)(F)F)C4)c3Cl)CC1)CN([C@@H](CCC(=O)OC(C)(C)C)C(N)=O)C2=O]

To a stirred solution of (3S)-1-(2,2,2-trifluoroethyl)pyrrolidin-3-ol (113 mg, 0.634 mmol) in dry THF (1.4 mL) were added successively tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.211 mmol), triphenylphosphane (166 mg, 0.634 mmol), and then DIAD (125 μL, 0.634 mmol). The reaction mixture was stirred at ambient temperature for 16 h. The reaction mixture was concentrated under reduced pressure. The crude was purified by flash chromatography on silica gel using a gradient of EtOAc/EtOH (3/1) (0.5% conc. NH₄OH) in n-heptane from 5% to 100%. The desired fractions were combined and concentrated under reduced pressure to afford tert-butyl(4S)-5-amino-4-[5-[4-[(R)-[2-chloro-3-[(3R)-1-(2,2,2-trifluoroethyl)pyrrolidin-3-yl]oxy-phenyl]-phenyl-methyl]piperidine-1-carbonyl]-6-methyl-1-oxo-isoindolin-2-yl]-5-oxo-pentanoate as a white solid (121 mg, 90% p., 64% y.). m/z [M+H]⁺=811.4. [α]_{D}=+15°·mL·dm⁻¹ g⁻¹[20° C., Na lamp, 589 nm, C=1.4 mg/mL, MeOH].

### Example 52

In a sealed vial, a solution of tert-butyl(4S)-5-amino-4-[5-[4-[(R)-[2-chloro-3-[(3R)-1-(2,2,2-trifluoroethyl)pyrrolidin-3-yl]oxy-phenyl]-phenyl-methyl]piperidine-1-carbonyl]-6-methyl-1-oxo-isoindolin-2-yl]-5-oxo-pentanoate (115 mg, 0.142 mmol) and 4-methylbenzenesulfonic acid hydrate (80.9 mg, 0.425 mmol) in acetonitrile (1.2 mL) was stirred at 85° C. for 3 h. The mixture was cooled to ambient temperature. EtOAc and water were added to the mixture. The mixture was poured into saturated aqueous NaHCO₃ and extracted with EtOAc (2×). The combined organic layers were washed with brine, filtered through hydrophobic paper, and concentrated. The crude was purified by reverse-phase preparative chromatography using a gradient of acetonitrile in water from 0% to 100% (0.1% AcOH). The desired fractions were combined, and acetonitrile evaporated. Saturated aqueous NaHCO₃ was added and the mixture was extracted with EtOAc (2×). The combined organic layers were washed with brine, filtered through hydrophobic paper, and concentrated. The residue was solubilized in the minimum amount of acetone and was treated with a 2 N solution of HCl in Et₂O(200 μL). The resulting suspension was diluted with Et₂O, stirred at ambient temperature for 2 h, and filtered. The residue was washed with Et₂O and dried under reduced pressure at 50° C. for 16 h to afford (3S)-3-[5-[4-[(R)-[2-chloro-3-[(3R)-1-(2,2,2-trifluoroethyl)pyrrolidin-3-yl]oxy-phenyl]-phenyl-methyl]piperidine-1-carbonyl]-6-methyl-1-oxo-isoindolin-2-yl]piperidine-2,6-dione as a white solid (37.5 mg, 100% p., 36% y.). m/z [M+H]⁺=737.3, ¹H NMR (600 MHz, DMSO-d₆): δ (ppm) 10.97 (s, 1H), 7.63-7.57 (m, 1H), 7.52-7.20 (m, 7H), 7.20-7.08 (m, 1H), 6.97-6.79 (m, 1H), 5.22-4.98 (m, 1H), 4.96-4.79 (m, 1H), 4.63-4.46 (m, 1H), 4.46-4.36 (m, 1H), 4.34-4.23 (m, 1H), 4.23-4.14 (m, 1H), 3.37-3.28 (m, 2H), 3.27-3.07 (m, 2H), 3.04-2.84 (m, 3H), 2.84-2.70 (m, 3H), 2.66-2.52 (m, 2H), 2.46-2.17 (m, 5H), 2.02-1.74 (m, 2H), 1.68-0.78 (m, 4H), [α]_{D}=+11°·mL·dm⁻¹ g⁻¹[20° C., Na lamp, 589 nm, C=1.3 mg/mL, DMSO].

### Example 53

### (3S)-3-[5-[4-[(S)-[2-chloro-3-(6,8-dihydro-5H-imidazo[1,5-a]pyrazin-7-yl)phenyl]-phenyl-methyl]piperidine-1-carbonyl]-6-methyl-1-oxo-isoindolin-2-yl]piperidine-2,6-dione

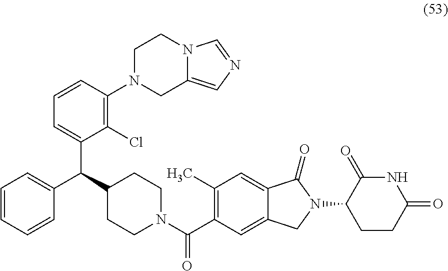 [SMILES: Cc1cc2c(cc1C(=O)N1CCC([C@@H](c3ccccc3)c3cccc(N4CCn5cncc5C4)c3Cl)CC1)CN([C@H]1CCC(=O)NC1=O)C2=O]

### Intermediate 53A: benzyl 4-[rel-(S)-[2-chloro-3-(6,8-dihydro-5H-imidazo[1,5-a]pyrazin-7-yl)phenyl]-phenyl-methyl]piperidine-1-carboxylate

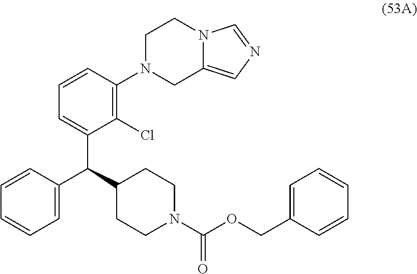 [SMILES: O=C(OCc1ccccc1)N1CCC([C@@H](c2ccccc2)c2cccc(N3CCn4cncc4C3)c2Cl)CC1]

In a sealed vial, to a stirred solution of benzyl 4-[(S)-(3-bromo-2-chloro-phenyl)-phenyl-methyl]piperidine-1-carboxylate (150 mg, 0.286 mmol) in dry 1,4-dioxane (2.0 mL) were added 4H,5H,6H,7H-imidazo[1,5-a]pyrazine (73.3 mg, 0.571 mmol), cesium carbonate (285 mg, 0.857 mmol), and [1-(2-diphenylphosphanyl-1-naphthyl)-2-naphthyl]-diphenyl-phosphane (17.8 mg, 0.0286 mmol) under argon. The reaction vessel was purged with argon 10 min after which Pd(OAc)₂(7.8 mg, 0.0343 mmol) was added. The reaction mixture was stirred at 110° C. for 18 h. The suspension was filtered through a pad of talcum powder which was then rinsed with EtOAc. The filtrate was concentrated under reduced pressure. The crude was purified by flash chromatography on silica gel using a gradient of EtOAc/EtOH (3/1) in n-heptane from 5% to 100%. The desired fractions were combined and concentrated under reduced pressure to afford (benzyl 4-[(S)-[2-chloro-3-(6,8-dihydro-5H-imidazo[1,5-a]pyrazin-7-yl)phenyl]-phenyl-methyl]piperidine-1-carboxylate as a pale yellow solid (43.0 mg, 97% p., 27% y.). m/z [M+H]⁺=541.3, ¹H NMR (400 MHz, DMSO-d₆) δ (ppm) 7.58 (d, J=1.0 Hz, 1H), 7.45 (dd, J=8.0, 1.5 Hz, 1H), 7.40-7.25 (m, 10H), 7.19-7.14 (m, 1H), 7.11 (dd, J=8.0, 1.4 Hz, 1H), 6.70 (d, J=1.1 Hz, 1H), 5.04 (s, 2H), 4.23 (d, J=11.1 Hz, 1H), 4.15 (s, 2H), 4.11 (t, J=5.4 Hz, 2H), 3.96 (d, J=13.1 Hz, 2H), 2.78 (s, 3H), 2.49 (s, 2H), 1.47 (d, J=13.1 Hz, 1H), 1.38 (d, J=13.4 Hz, 1H), 1.01 (dd, J=22.7, 11.7 Hz, 2H), [α]_{D}=−64°·mL·dm⁻¹ g^{-1 [20}° C., Na lamp, 589 nm, C=1.4 mg/mL, MeOH].

### Intermediate 53B: 7-[2-chloro-3-[rel-(S)-phenyl(4-piperidyl)methyl]phenyl]-6,8-dihydro-5H-imidazo[1,5-a]pyrazine trihydroiodide

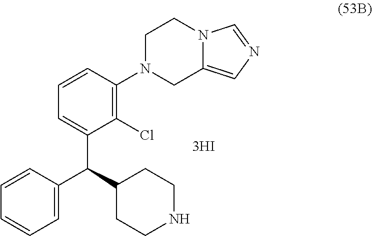 [SMILES: Clc1c([C@H](c2ccccc2)C2CCNCC2)cccc1N1CCn2cncc2C1]

To a stirred solution of benzyl 4-[(S)-[2-chloro-3-(6,8-dihydro-5H-imidazo[1,5-a]pyrazin-7-yl)phenyl]-phenyl-methyl]piperidine-1-carboxylate (40.0 mg, 0.0717 mmol) in acetonitrile (750 μL) was added iodo (trimethyl) silane (30.6 μL, 0.215 mmol). The reaction mixture was stirred at ambient temperature for 15 min. The mixture was cooled to 0° C. and MeOH (34.9 μL, 0.861 mmol) was added. The mixture was stirred at room temperature for 2 h and was concentrated under reduced pressure. Et₂O was added and the resulting mixture was stirred at ambient temperature for 1 h. The solid was filtered, washed with Et₂O, and dried under reduced pressure for 72 h to afford (7-[2-chloro-3-[(S)-phenyl(4-piperidyl)methyl]phenyl]-6,8-dihydro-5H-imidazo[1,5-a]pyrazine trihydroiodide as a yellow solid (33 mg, 92% p., 54% y.). m/z [M+H]⁺=407.4, ¹H NMR (400 MHz, DMSO-d₆): δ (ppm) 14.22 (s, 1H), 9.02 (s, 1H), 8.40 (s, 1H), 8.09 (s, 1H), 7.50-7.45 (m, 2H), 7.40 (d, J=8.2 Hz, 1H), 7.39-7.35 (m, 2H), 7.31 (t, J=7.6 Hz, 2H), 7.23-7.19 (m, 1H), 7.17 (dd, J=7.8, 1.4 Hz, 1H), 4.36 (d, J=5.5 Hz, 1H), 4.28 (d, J=4.4 Hz, 2H), 4.21 (d, J=11.3 Hz, 1H), 3.45 (t, J=5.5 Hz, 2H), 3.24 (t, J=11.6 Hz, 3H), 2.84 (t, J=11.9 Hz, 2H), 2.67-2.54 (m, 1H), 1.59 (d, J=14.1 Hz, 1H), 1.52 (d, J=14.1 Hz, 1H), 1.25 (t, J=15.1 Hz, 3H), [α]_{D}=−27°·mL·dm⁻¹ g⁻¹[20° C., Na lamp, 589 nm, C=1.1 mg/mL, MeOH].

### Intermediate 53C: tert-butyl(4S)-5-amino-4-[5-[4-[(S)-[2-chloro-3-(6,8-dihydro-5H-imidazo[1,5-a]pyrazin-7-yl)phenyl]-phenyl-methyl]piperidine-1-carbonyl]-6-methyl-1-oxo-isoindolin-2-yl]-5-oxo-pentanoate

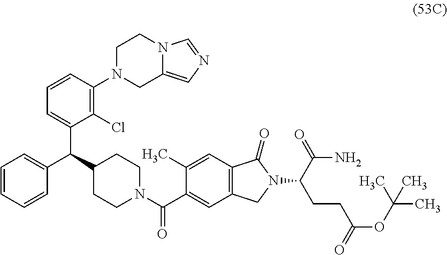 [SMILES: Cc1cc2c(cc1C(=O)N1CCC([C@@H](c3ccccc3)c3cccc(N4CCn5cncc5C4)c3Cl)CC1)CN([C@@H](CCC(=O)OC(C)(C)C)C(N)=O)C2=O]

To a solution of 7-[2-chloro-3-[(S)-phenyl(4-piperidyl)methyl]phenyl]-6,8-dihydro-5H-imidazo[1,5-a]pyrazine trihydroiodide (33.0 mg, 0.0384 mmol) in DMF (150 μL), were added 2-[(1S)-4-tert-butoxy-1-carbamoyl-4-oxo-butyl]-6-methyl-1-oxo-isoindoline-5-carboxylic acid (15.5 mg, 0.0403 mmol), DIPEA (33.5 μL, 0.192 mmol) and HATU (22.6 mg, 0.0576 mmol). The mixture was stirred at ambient temperature for 16 h. The reaction was quenched with the addition of a saturated aqueous solution of NaHCO₃. EtOAc was added to the mixture. The aqueous layer was extracted with EtOAc (2×). The combined organic layers were washed with a saturated aqueous solution of NH₄Cl, then brine, dried over Na₂SO₄, filtered, and concentrated under reduced pressure. The crude was purified by flash chromatography on silica gel using a gradient of MeOH in DCM from 0% to 20%. The desired fractions were combined and concentrated under reduced pressure to afford (tert-butyl(4S)-5-amino-4-[5-[4-[(S)-[2-chloro-3-(6,8-dihydro-5H-imidazo[1,5-a]pyrazin-7-yl)phenyl]-phenyl-methyl]piperidine-1-carbonyl]-6-methyl-1-oxo-isoindolin-2-yl]-5-oxo-pentanoate as a yellow solid (33.3 mg, 74% p., 84% y.). m/z [M+H]⁺=765.5.

### Example 53

In a sealed vial, a solution of tert-butyl(4S)-5-amino-4-[5-[4-[(S)-[2-chloro-3-(6,8-dihydro-5H-imidazo[1,5-a]pyrazin-7-yl)phenyl]-phenyl-methyl]piperidine-1-carbonyl]-6-methyl-1-oxo-isoindolin-2-yl]-5-oxo-pentanoate (31.0 mg, 0.0405 mmol) and 4-methylbenzenesulfonic acid hydrate (23.1 mg, 0.122 mmol) in acetonitrile (310 μL) was stirred at 85° C. for 3 h. The mixture was cooled to ambient temperature then EtOAc and water were added. The mixture was poured into a saturated aqueous solution of NaHCO₃ and extracted with EtOAc (2×). The combined organic layers were washed with brine, filtered through hydrophobic paper, and concentrated. The crude was purified by reverse-phase preparative chromatography using a gradient of acetonitrile in water from 0% to 100% (0.1% AcOH). The desired fractions were combined, and acetonitrile evaporated. A saturated aqueous solution of NaHCO₃ was added, the suspension was stirred at ambient temperature for 2 h, and filtered. The residue was washed with water and dried under reduced pressure at 50° C. for 16 h to afford (35)-3-[5-[4-[(S)-[2-chloro-3-(6,8-dihydro-5H-imidazo[1,5-a]pyrazin-7-yl)phenyl]-phenyl-methyl]piperidine-1-carbonyl]-6-methyl-1-oxo-isoindolin-2-yl]piperidine-2,6-dione as a white solid (3.0 mg, 91.7% p., 10% y.). m/z [M+H]⁺=691.3, ¹H NMR (600 MHz, DMSO-d₆): δ (ppm) 11.01-10.92 (m, 1H), 7.64-7.56 (m, 2H), 7.48 (br s, 1H), 7.43 (br s, 1H), 7.41-7.37 (m, 1H), 7.36-7.31 (m, 2H), 7.31-7.22 (m, 2H), 7.22-7.14 (m, 1H), 7.13-7.05 (m, 1H), 6.69 (dd, J=15.5, 0.8 Hz, 1H), 5.09 (br dd, J=13.1, 4.5 Hz, 1H), 4.58-4.48 (m, 1H), 4.47-4.36 (m, 1H), 4.34-4.22 (m, 2H), 4.20-4.12 (m, 2H), 4.09 (s, 2H), 3.36-3.31 (m, 2H), 3.27-3.16 (m, 1H), 3.05-2.93 (m, 1H), 2.93-2.81 (m, 1H), 2.76 (d, J=6.9 Hz, 1H), 2.64-2.52 (m, 2H), 2.47-2.35 (m, 1H), 2.25 (s, 3H), 2.04-1.92 (m, 1H), 1.67-1.27 (m, 2H), 1.27-1.13 (m, 1H), 1.06-0.91 (m, 1H).

### Example 54

### (3S)-3-[5-[4-[(R)-[2-chloro-3-[(3R)-1-(2,2-difluoroethyl)pyrrolidin-3-yl]oxy-phenyl]-phenyl-methyl]piperidine-1-carbonyl]-6-methyl-1-oxo-isoindolin-2-yl]piperidine-2,6-dione

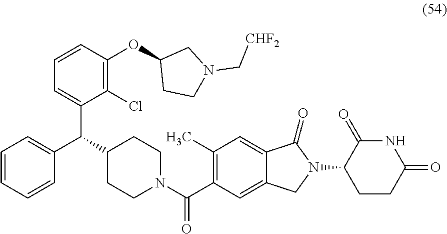 [SMILES: Cc1cc2c(cc1C(=O)N1CCC([C@H](c3ccccc3)c3cccc(O[C@@H]4CCN(CC(F)F)C4)c3Cl)CC1)CN([C@H]1CCC(=O)NC1=O)C2=O]

### Intermediate 54A: tert-butyl(4S)-5-amino-4-[5-[4-[(R)-[2-chloro-3-[(3R)-1-(2,2-difluoroethyl)pyrrolidin-3-yl]oxy-phenyl]-phenyl-methyl]piperidine-1-carbonyl]-6-methyl-1-oxo-isoindolin-2-yl]-5-oxo-pentanoate

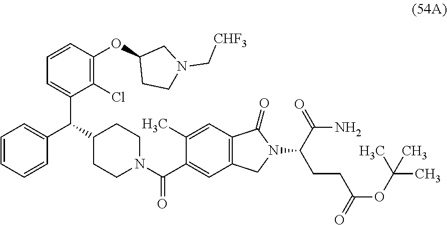

To a stirred solution of (3S)-1-(2,2-difluoroethyl)pyrrolidin-3-ol (101 mg, 0.634 mmol) in dry THF (1.4 mL) were added successively tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.211 mmol), triphenylphosphane (166 mg, 0.634 mmol), and then DIAD (125 μL, 0.634 mmol). The reaction mixture was stirred at ambient temperature for 16 h. The reaction mixture was concentrated under reduced pressure. The crude was purified by flash chromatography on silica gel using a gradient of EtOAc/EtOH (3/1) (0.5% conc. NH₄OH) in n-heptane from 5% to 100%. The desired fractions were combined and concentrated under reduced pressure to afford tert-butyl (4S)-5-amino-4-[5-[4-[(R)-[2-chloro-3-[(3R)-1-(2,2-difluoroethyl)pyrrolidin-3-yl]oxy-phenyl]-phenyl-methyl]piperidine-1-carbonyl]-6-methyl-1-oxo-isoindolin-2-yl]-5-oxo-pentanoate as a white solid (130 mg, 98% p., 76% y.). m/z [M+H]⁺=793.5. [α]_{D}=+16°·mL·dm⁻¹ g⁻¹[20° C., Na lamp, 589 nm, C=1.8 mg/mL, MeOH].

### Example 54

In a sealed vial, to a solution of tert-butyl(4S)-5-amino-4-[5-[4-[(R)-[2-chloro-3-[(3R)-1-(2,2-difluoroethyl)pyrrolidin-3-yl]oxy-phenyl]-phenyl-methyl]piperidine-1-carbonyl]-6-methyl-1-oxo-isoindolin-2-yl]-5-oxo-pentanoate (124 mg, 0.156 mmol) in acetonitrile (2.7 mL) was added 4-methylbenzenesulfonic acid hydrate (89.2 mg, 0.469 mmol). The solution was stirred at 90° C. for 4 h. The mixture was diluted with EtOAc and washed with a saturated aqueous solution of NaHCO₃ and brine. The organic layer was dried over Na₂SO₄, filtered, and concentrated under reduced pressure. The crude was purified by reverse-phase preparative chromatography using a gradient of acetonitrile in water from 0% to 100% (0.1% AcOH). The desired fractions were combined and concentrated under reduced pressure until evaporation of most of the acetonitrile. A saturated aqueous solution of NaHCO₃ was added. The resulting suspension was filtered and dried under at 50° C. under reduced pressure overnight to afford (3S)-3-[5-[4-[(R)-[2-chloro-3-[(3R)-1-(2,2-difluoroethyl)pyrrolidin-3-yl]oxy-phenyl]-phenyl-methyl]piperidine-1-carbonyl]-6-methyl-1-oxo-isoindolin-2-yl]piperidine-2,6-dione as a white powder (3.2 mg, 97.8% p., 3% y.). m/z [M+H]⁺=719.3, ¹H NMR (600 MHz, DMSO-d₆): δ (ppm) 10.97 (s, 1H), 7.65-7.56 (m, 1H), 7.51-7.19 (m, 7H), 7.21-7.07 (m, 1H), 6.93-6.75 (m, 1H), 6.22-5.94 (m, 1H), 5.14-5.04 (m, 1H), 4.93-4.77 (m, 1H), 4.58-4.47 (m, 1H), 4.46-4.36 (m, 1H), 4.35-4.12 (m, 2H), 3.27-3.11 (m, 1H), 3.04-2.92 (m, 2H), 2.92-2.67 (m, 6H), 2.66-2.52 (m, 3H), 2.44-2.17 (m, 5H), 2.04-1.92 (m, 1H), 1.88-1.70 (m, 1H), 1.67-0.80 (m, 4H).

### Example 55

### (3S)-3-[5-[4-[(R)-[2-chloro-3-(cyclobutoxy)phenyl]-phenyl-methyl]piperidine-1-carbonyl]-6-methyl-1-oxo-isoindolin-2-yl]piperidine-2,6-dione

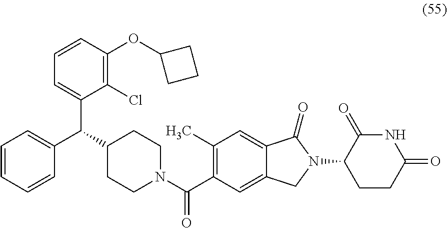 [SMILES: Cc1cc2c(cc1C(=O)N1CCC([C@H](c3ccccc3)c3cccc(OC4CCC4)c3Cl)CC1)CN([C@H]1CCC(=O)NC1=O)C2=O]

### Intermediate 55A: tert-butyl(4S)-5-amino-4-[5-[4-[(R)-[2-chloro-3-(cyclobutoxy) phenyl]-phenyl-methyl]piperdine-1-carbonyl]-6-methyl-1-oxo-isoindolin-2-yl]-5-oxo pentanoate

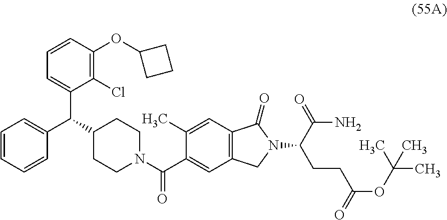 [SMILES: Cc1cc2c(cc1C(=O)N1CCC([C@H](c3ccccc3)c3cccc(OC4CCC4)c3Cl)CC1)CN([C@@H](CCC(=O)OC(C)(C)C)C(N)=O)C2=O]

To a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.211 mmol) in dry THF (1.4 mL) were added successively triphenylphosphane (166 mg, 0.634 mmol), cyclobutanol (47.1 mg, 0.634 mmol), and then DIAD (125 μL, 0.634 mmol). The reaction mixture was stirred at ambient temperature for 16 h. The reaction mixture was concentrated under reduced pressure. The crude was purified by flash chromatography on silica gel using a gradient of EtOAc/EtOH (3/1) (0.5% conc. NH₄OH) in n-heptane from 5% to 100%. The desired fractions were combined and concentrated under reduced pressure. The residue was purified by reverse-phase preparative chromatography using a gradient of acetonitrile in water from 0% to 100% (0.1% AcOH). Acetonitrile was removed and a saturated aqueous solution of NaHCO₃ was added. The aqueous layer was extracted with DCM. The combined organic layers were washed with brine, dried over Na₂SO₄, filtered, and concentrated under reduced pressure to afford tert-butyl(4S)-5-amino-4-[5-[4-[(R)-[2-chloro-3-(cyclobutoxy)phenyl]-phenyl-methyl]piperidine-1-carbonyl]-6-methyl-1-oxo-isoindolin-2-yl]-5-oxo-pentanoate as a white solid (70.8 mg, 80% p., 38% y.). m/z [M+H]⁺=714.5. [α]_{D}=+17°·mL·dm⁻¹·g⁻¹[20° C., Na lamp, 589 nm, C=1.5 mg/mL, MeOH].

### Example 55

In a sealed vial, to a solution of tert-butyl(4S)-5-amino-4-[5-[4-[(R)-[2-chloro-3-(cyclobutoxy)phenyl]-phenyl-methyl]piperidine-1-carbonyl]-6-methyl-1-oxo-isoindolin-2-yl]-5-oxo-pentanoate (52.0 mg, 0.0728 mmol) in acetonitrile (1.2 mL) was added 4-methylbenzenesulfonic acid hydrate (41.5 mg, 0.218 mmol). The solution was stirred at 90° C. for 3 h. The mixture was diluted with EtOAc and washed with a saturated aqueous solution of NaHCO₃ and brine. The organic layer was dried over Na₂SO₄, filtered, and concentrated under reduced pressure. The crude was purified by reverse-phase preparative chromatography using a gradient of acetonitrile in water from 0% to 100% (0.1% AcOH). The desired fractions were combined and concentrated under reduced pressure until evaporation of most of the acetonitrile. A saturated aqueous solution of NaHCO₃ was added. The resulting suspension was filtered and dried under at 50° C. under reduced pressure overnight to afford (3S)-3-[5-[4-[(R)-[2-chloro-3-(cyclobutoxy)phenyl]-phenyl-methyl]piperidine-1-carbonyl]-6-methyl-1-oxo-isoindolin-2-yl]piperidine-2,6-dione as a white powder (20.7 mg, 97.2% p., 43% y.). m/z [M+H]⁺=640.2, ¹H NMR (600 MHz, DMSO-d₆): δ (ppm) 10.97 (s, 1H), 7.65-7.56 (m, 1H), 7.54-7.06 (m, 8H), 6.82-6.70 (m, 1H), 5.15-5.03 (m, 1H), 4.74-4.60 (m, 1H), 4.56-4.46 (m, 1H), 4.46-4.13 (m, 3H), 3.27-3.14 (m, 1H), 3.03-2.83 (m, 2H), 2.82-2.69 (m, 1H), 2.65-2.51 (m, 2H), 2.47-2.21 (m, 6H), 2.14-1.92 (m, 3H), 1.83-1.70 (m, 1H), 1.70-0.89 (m, 5H), [α]_{D}=+11°·mL·dm⁻¹·g⁻¹[20° C., Na lamp, 589 nm, C=1.0 mg/mL, DMSO].

### Example 56

### N-[2-chloro-3-[1-[2-[(3S)-2,6-dioxo-3-piperidyl]-6-methyl-1-oxo-isoindoline-5-carbonyl]-4-piperidyl]-phenyl-methyl]phenyl]-2-hydroxy-acetamide

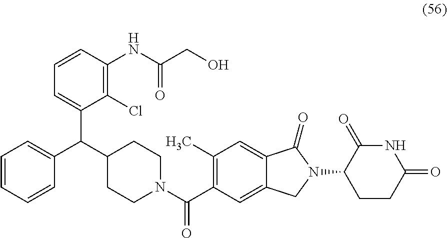 [SMILES: Cc1cc2c(cc1C(=O)N1CCC(C(c3ccccc3)c3cccc(NC(=O)CO)c3Cl)CC1)CN([C@H]1CCC(=O)NC1=O)C2=O]

### Intermediate 56A: benzyl 4-[[2-chloro-3-[(2-hydroxyacetyl)amino]phenyl]-phenyl-methyl]piperidine-1-carboxylate

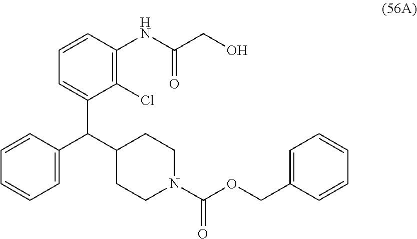 [SMILES: O=C(CO)Nc1cccc(C(c2ccccc2)C2CCN(C(=O)OCc3ccccc3)CC2)c1Cl]

A stirred solution of benzyl 4-[(3-bromo-2-chloro-phenyl)-phenyl-methyl]piperidine-1-carboxylate (120 mg, 0.241 mmol) and 2-hydroxyacetamide (37.2 mg, 0.481 mmol) in 1,4-dioxane (1.2 mL) at ambient temperature was degassed with argon for 10 min, t-BuONa (27.7 mg, 0.289 mmol), and Brettphos-Pd-G1 (20.3 mg, 0.0241 mmol) were added. The reaction mixture was stirred at 80° C. overnight. EtOAc was added and the mixture was washed with a saturated aqueous solution of NH₄Cl, water, brine, dried over Na₂SO₄, filtered, and concentrated under reduced pressure. The crude was purified by flash chromatography on silica gel using a gradient of EtOAc in n-heptane from 0% to 100%. The desired fractions were combined and concentrated under reduced pressure to afford (benzyl 4-[2-chloro-3-[(2-hydroxyacetyl)amino]phenyl]-phenyl-methyl]piperidine-1-carboxylate as a white solid (63.0 mg, 92% p., 49% y.). m/z [M+H]⁺=493.2, ¹H NMR (400 MHz, DMSO-d₆): δ (ppm) 9.34 (s, 1H), 8.05 (dd, J=8.0, 1.5 Hz, 1H), 7.50 (dd, J=7.9, 1.6 Hz, 1H), 7.45-7.23 (m, 10H), 7.21-7.13 (m, 1H), 6.15 (t, J=5.7 Hz, 1H), 5.05 (s, 2H), 4.15 (d, J=11.2 Hz, 1H), 4.01-3.90 (m, 3H), 2.81-2.76 (m, 3H), 1.99 (s, 1H), 1.49 (d, J=13.2 Hz, 1H), 1.37 (d, J=13.1 Hz, 1H), 1.03 (dq, J=23.4, 10.7 Hz, 2H).

### Intermediate 56B: N-[2-chloro-3-[phenyl(4-piperidyl)methyl]phenyl]-2-hydroxy-acetamide hydroiodide

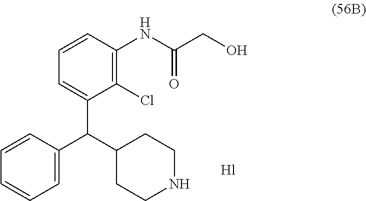 [SMILES: O=C(CO)Nc1cccc(C(c2ccccc2)C2CCNCC2)c1Cl ; "Hl"]

To a stirred solution of benzyl 4-[2-chloro-3-[(2-hydroxyacetyl)amino]phenyl]-phenyl-methyl]piperidine-1-carboxylate (106 mg, 0.198 mmol) in acetonitrile (1.4 mL) at ambient temperature under nitrogen was added iodo (trimethyl) silane (84.5 μL, 0.593 mmol) dropwise. The reaction mixture was stirred at ambient temperature for 1 h. The mixture was cooled to 0° C. and MeOH (96.2 μL, 2.37 mmol) was added dropwise. The mixture was stirred at ambient temperature for 1 h and was concentrated under reduced pressure. Et₂O was added and the resulting mixture was stirred at ambient temperature for 1 h. The solid was filtered, washed with Et₂O, and dried under reduced pressure overnight to afford (N-[2-chloro-3-[phenyl(4-piperidyl)methyl]phenyl]-2-hydroxy-acetamide hydroiodide as a light brown solid (89.0 mg, 88% p., 81% y.). m/z [M+H]⁺=359.3.

### Intermediate 56C: tert-butyl(4S)-5-amino-4-[5-[4-[[2-chloro-3-[(2-hydroxyacetyl)amino]phenyl]-phenyl-methyl]piperidine-1-carbonyl]-6-methyl-1-oxo-isoindolin-2-yl]-5-oxo-pentanoate pentanoate

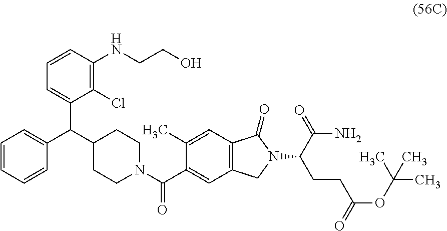 [SMILES: Cc1cc2c(cc1C(=O)N1CCC(C(c3ccccc3)c3cccc(NCCO)c3Cl)CC1)CN([C@@H](CCC(=O)OC(C)(C)C)C(N)=O)C2=O]

To a solution of N-[2-chloro-3-[phenyl(4-piperidyl)methyl]phenyl]-2-hydroxy-acetamide hydroiodide (86.0 mg, 0.161 mmol) in dry DMF (0.9 mL), were added 2-[(1S)-4-tert-butoxy-1-carbamoyl-4-oxo-butyl]-6-methyl-1-oxo-isoindoline-5-carboxylic acid (63.5 mg, 0.169 mmol), DIPEA (112 μL, 0.643 mmol), and HATU (93.6 mg, 0.241 mmol). The mixture was stirred at ambient temperature overnight. The reaction was quenched with the addition of water. The mixture was basified with a saturated aqueous solution of NaHCO₃. EtOAc and then FastWorX® powder were added. The organic layer was concentrated under reduced pressure. The reaction mixture was filtered through a dry-loading cartridge and was rinsed with water. The crude was purified by flash chromatography on silica gel using a gradient of EtOAc/EtOH (3/1) in n-heptane from 5% to 100% to afford tert-butyl(4S)-5-amino-4-[5-[4-[[2-chloro-3-[(2-hydroxyacetyl) amino]phenyl]-phenyl-methyl]piperidine-1-carbonyl]-6-methyl-1-oxo-isoindolin-2-yl]-5-oxo-pentanoate as a white solid (80.0 mg, 80% p., 56% y.). m/z [M+H]⁺=717.3.

### Example 56

To a stirred solution of tert-butyl(4S)-5-amino-4-[5-[4-[2-chloro-3-[(2-hydroxyacetyl)amino]phenyl]-phenyl-methyl]piperidine-1-carbonyl]-6-methyl-1-oxo-isoindolin-2-yl]-5-oxo-pentanoate (80.0 mg, 0.0892 mmol) in acetonitrile (1.0 mL) at ambient temperature was added 4-methylbenzenesulfonic acid hydrate (50.9 mg, 0.268 mmol). The reaction mixture was stirred at 80° C. for 3 h. The reaction was quenched with the addition of a saturated aqueous solution of NaHCO₃. EtOAc was added to the mixture. The aqueous layer was extracted with EtOAc (2×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated, and the aqueous layer was basified with a saturated aqueous solution of NaHCO₃. The resulting precipitate was filtered, washed with water, and dried under reduced pressure at 50° C. to afford N-[2-chloro-3-[1-[2-[(3S)-2,6-dioxo-3-piperidyl]-6-methyl-1-oxo-isoindoline-5-carbonyl]-4-piperidyl]-phenyl-methyl]phenyl]-2-hydroxy-acetamide as a white powder (7.4 mg, 98.5% p., 13% y.). m/z [M+H]⁺=643.2, ¹H NMR (600 MHz, DMSO-d₆): δ (ppm) 10.97 (s, 1H), 9.43-9.22 (m, 1H), 8.13-7.93 (m, 1H), 7.69-6.98 (m, 9H), 6.22-6.07 (m, 1H), 5.17-5.02 (m, 1H), 4.64-4.08 (m, 4H), 4.08-3.88 (m, 2H), 3.26-3.11 (m, 1H), 3.09-2.69 (m, 3H), 2.66-2.52 (m, 2H), 2.45-2.30 (m, 2H), 2.25 (s, 2H), 2.06-1.92 (m, 1H), 1.69-0.91 (m, 4H).

### Example 57

### N-[2-[2-chloro-3-[(R)-[1-[2-[(3S)-2,6-dioxo-3-piperidyl]-6-methyl-1-oxo-isoindoline-5-carbonyl]-4-piperidyl]-phenyl-methyl]phenoxy]ethyl]acetamide

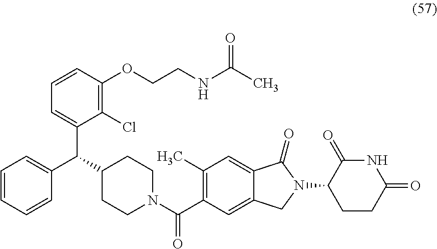 [SMILES: CC(=O)NCCOc1cccc([C@@H](c2ccccc2)C2CCN(C(=O)c3cc4c(cc3C)C(=O)N([C@H]3CCC(=O)NC3=O)C4)CC2)c1Cl]

### Intermediate 57A: benzyl 4-[(R)-[3-[2-(tert-butoxycarbonylamino) ethoxy]-2-chloro-phenyl]-phenyl-methyl]piperidine-1-carboxylate

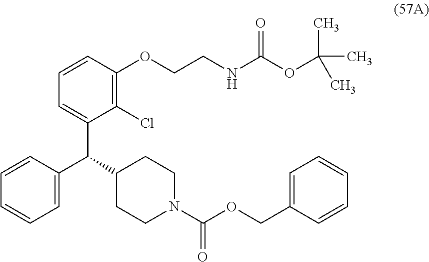 [SMILES: CC(C)(C)OC(=O)NCCOc1cccc([C@@H](c2ccccc2)C2CCN(C(=O)OCc3ccccc3)CC2)c1Cl]

To a stirred solution of benzyl 4-[(R)-(2-chloro-3-hydroxy-phenyl)-phenyl-methyl]piperidine-1-carboxylate (247 mg, 0.562 mmol) in dry THF (3.5 mL) were added successively triphenylphosphane (442 mg, 1.69 mmol), tert-butyl(2-hydroxyethyl) carbamate (261 μL, 1.69 mmol), and then DIAD (332 μL, 1.69 mmol). The reaction mixture was stirred at ambient temperature for 72 h. The reaction mixture was concentrated under reduced pressure. The crude was purified by flash chromatography on silica gel using a gradient of EtOAc/EtOH (3/1) (0.5% conc. NH₄OH) in n-heptane from 5% to 100% to afford benzyl 4-[(R)-[3-[2-(tert-butoxycarbonylamino) ethoxy]-2-chloro-phenyl]-phenyl-methyl]piperidine-1-carboxylate as a white powder (350 mg, 100% p., quant. yield)). m/z [M+Na]⁺=601.2, ¹H NMR (400 MHz, DMSO-d₆): δ (ppm) 7.39-7.20 (m, 10H), 7.20-7.12 (m, 1H), 6.92 (dt, J=12.4, 5.4 Hz, 2H), 6.62 (s, 1H), 5.05 (s, 2H), 4.79 (d, J=11.6 Hz, 2H), 4.17 (d, J=11.2 Hz, 1H), 3.97 (q, J=8.9 Hz, 4H), 3.29 (m, 2H), 3.13 (s, 1H), 3.04 (d, J=6.9 Hz, 1H), 2.78 (s, 2H), 1.54-1.43 (m, 1H), 1.37 (s, 9H).

### Intermediate 57B: benzyl 4-[(R)-[3-(2-aminoethoxy)-2-chloro-phenyl]-phenyl-methyl]piperidine-1-carboxylate hydrochloride

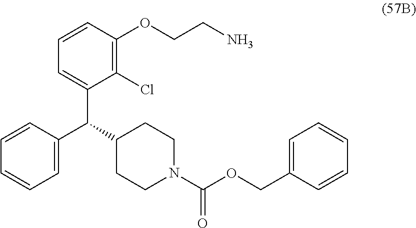 [SMILES: NCCOc1cccc([C@@H](c2ccccc2)C2CCN(C(=O)OCc3ccccc3)CC2)c1Cl]

To a stirred solution of benzyl 4-[(R)-[3-[2-(tert-butoxycarbonylamino) ethoxy]-2-chloro-phenyl]-phenyl-methyl]piperidine-1-carboxylate (350 mg, 0.604 mmol) in dry 1,4-dioxane (4.0 mL) at ambient temperature was added a 4 M solution of HCl in 1,4-dioxane (1.5 mL, 6.04 mmol). The reaction mixture was stirred at ambient temperature for 16 h. Et₂O was added and the resulting precipitate was stirred for 30 min, filtered, washed with Et₂O, and dried under reduced pressure at 50° C. for 16 h to afford (benzyl 4-[(R)-[3-(2-aminoethoxy)-2-chloro-phenyl]-phenyl-methyl]piperidine-1-carboxylate hydrochloride as a white powder (100 mg, 97% p., 31% y.). m/z [M+H]⁺=479.2, ¹H NMR (400 MHz, DMSO-d₆): δ (ppm) 7.89 (s, 3H), 7.33 (m, 11H), 7.16 (m, 1H), 7.00 (dd, J=2.0, 7.6 Hz, 1H), 5.05 (s, 2H), 4.82 (m, 2H), 4.18 (m, 3H), 3.96 (m, 2H), 3.22 (t, J=5.3 Hz, 2H), 2.92 (m, 1H), 2.79 (m, 2H), 1.03 (m, 2H).

### Intermediate 57C: benzyl 4-[(R)-[3-(2-acetamidoethoxy)-2-chloro-phenyl]-phenyl-methyl]piperidine-1-carboxylate

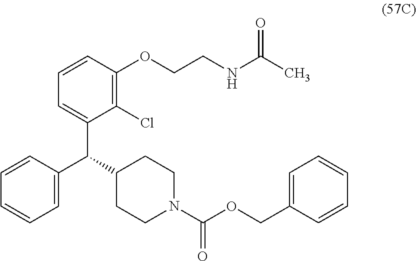 [SMILES: CC(=O)NCCOc1cccc([C@@H](c2ccccc2)C2CCN(C(=O)OCc3ccccc3)CC2)c1Cl]

To a stirred solution of benzyl 4-[(R)-[3-(2-aminoethoxy)-2-chloro-phenyl]-phenyl-methyl]piperidine-1-carboxylate hydrochloride (100 mg, 0.188 mmol) in THF (1.0 mL) at ambient temperature were added DIPEA (98.6 μL, 0.565 mmol) and then acetyl chloride (14.7 μL, 0.207 mmol). The reaction mixture was stirred at ambient temperature for 16 h. The reaction was quenched with the addition of a saturated aqueous solution of NaHCO₃. EtOAc was added to the mixture. The aqueous layer was extracted with EtOAc (2×). The combined organic layers were washed with brine, dried over Na₂SO₄, filtered, and concentrated under reduced pressure. The crude was purified by flash chromatography on silica gel using a gradient of EtOAc/EtOH (3/1) in cyclohexane from 5% to 50% to afford benzyl 4-[(R)-[3-(2-acetamidoethoxy)-2-chloro-phenyl]-phenyl-methyl]piperidine-1-carboxylate as a white solid (55.0 mg, 99% p., 56% y.). m/z [M+Na]⁺=543.3, ¹H NMR (400 MHz, DMSO-d₆): δ (ppm) 8.05-8.00 (m, 1H), 7.39-7.30 (m, 7H), 7.30-7.23 (m, 4H), 7.20-7.11 (m, 1H), 6.99-6.91 (m, 1H), 5.05 (s, 2H), 4.85-4.68 (m, 1H), 4.17 (d, J=11.2 Hz, 1H), 4.06-3.98 (m, 2H), 3.39 (m, 2H), 3.15 (d, J=6.5 Hz, 1H), 2.78 (s, 3H), 1.81 (s, 3H), 1.51-1.43 (m, 1H), 1.42-1.34 (m, 1H), 1.25 (s, 1H), 1.10-0.91 (m, 1H).

### Intermediate 57D: N-[2-[2-chloro-3-[(R)-phenyl(4-piperidyl)methyl]phenoxy]ethyl]acetamide hydroiodide

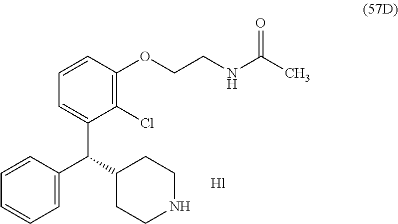

To a stirred solution of benzyl 4-[(R)-[3-(2-acetamidoethoxy)-2-chloro-phenyl]-phenyl-methyl]piperidine-1-carboxylate (55.0 mg, 0.105 mmol) in acetonitrile (1.0 mL) at ambient temperature under nitrogen was added iodo (trimethyl) silane (44.6 μL, 0.314 mmol). The reaction mixture was stirred at ambient temperature for 30 min. MeOH (50.8 μL, 1.25 mmol) was added at 0° C. The mixture was stirred at ambient temperature for 2 h and was concentrated under reduced pressure. Et₂O was added and the resulting mixture was stirred at ambient temperature for 2 h. The solid was filtered, washed with Et₂O, and dried under reduced pressure to afford N-[2-[2-chloro-3-[(R)-phenyl(4-piperidyl)methyl]phenoxy]ethyl]acetamide hydroiodide as an orange solid (34.0 mg, 94% p., 59% y.). m/z [M+H]⁺=387.3, ¹H NMR (400 MHz, DMSO-d₆): δ (ppm) 8.40 (s, 1H), 8.05-7.98 (m, 1H), 7.38-7.32 (m, 2H), 7.34-7.26 (m, 3H), 7.25-7.13 (m, 2H), 6.99 (d, J=8.6 Hz, 1H), 4.17 (d, J=11.3 Hz, 1H), 4.06-3.94 (m, 2H), 3.44-3.33 (m, 3H), 3.23 (m, 2H), 2.89-2.76 (m, 2H), 2.57 (d, J=11.3 Hz, 1H), 1.81 (s, 3H), 1.61-1.49 (m, 2H), 1.38-1.12 (m, 2H).

### Intermediate 57E: tert-butyl(4S)-4-[5-[4-[(R)-[3-(2-acetamidoethoxy)-2-chloro-phenyl]-phenyl-methyl]piperidine-1-carbonyl]-6-methyl-1-oxo-isoindolin-2-yl]-5-amino-5-oxo-pentanoate

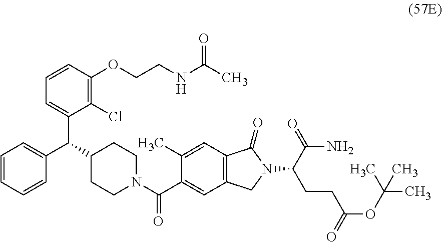 [SMILES: CC(=O)NCCOc1cccc([C@@H](c2ccccc2)C2CCN(C(=O)c3cc4c(cc3C)C(=O)N([C@@H](CCC(=O)OC(C)(C)C)C(N)=O)C4)CC2)c1Cl]

To a stirred solution of 2-[(1S)-4-tert-butoxy-1-carbamoyl-4-oxo-butyl]-6-methyl-1-oxo-isoindoline-5-carboxylic acid (25.7 mg, 0.0683 mmol) in dry DCM (500 μL) at ambient temperature were added N-[2-[2-chloro-3-[(R)-phenyl(4-piperidyl)methyl]phenoxy]-ethyl]acetamide hydroiodide (34.0 mg, 0.0621 mmol), DIPEA (43.4 μL, 0.248 mmol), and then a solution of T3P in DCM (53% w/w, 52.3 μL, 0.0931 mmol). The reaction mixture was stirred at ambient temperature for 20 min. The reaction was quenched with the addition of a saturated aqueous solution of NH₄Cl. DCM was added to the mixture. The layers were separated and the aqueous layer was extracted with DCM. The combined organic layers were washed with brine, dried over Na₂SO₄, filtered, and concentrated under reduced pressure. The crude was purified by flash chromatography on silica gel using a gradient of EtOAc/EtOH (3/1) in n-heptane from 10% to 100% to afford tert-butyl(4S)-4-[5-[4-[(R)-[3-(2-acetamidoethoxy)-2-chloro-phenyl]-phenyl-methyl]piperidine-1-carbonyl]-1-oxo-isoindolin-2-yl]-5-amino-5-oxo-pentanoate as a white solid (20.0 mg, 97% p., 42% y.). m/z [M+H]⁺=745.4.

### Example 57

To a stirred solution of tert-butyl(4S)-4-[5-[4-[(R)-[3-(2-acetamidoethoxy)-2-chloro-phenyl]-phenyl-methyl]piperidine-1-carbonyl]-6-methyl-1-oxo-isoindolin-2-yl]-5-amino-5-oxo-pentanoate (19.4 mg, 0.0260 mmol) in dry acetonitrile (500 μL) at ambient temperature was added 4-methylbenzenesulfonic acid hydrate (14.9 mg, 0.0781 mmol). The reaction mixture was stirred at 80° C. for 5 h. The reaction was quenched with the addition of a saturated aqueous solution of NaHCO₃. EtOAc was added to the mixture. The aqueous layer was extracted with EtOAc (2×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated and aqueous layer was basified with a saturated aqueous solution of NaHCO₃. The aqueous layer was extracted with EtOAc (2×). The combined organic layers were washed with brine, dried over Na₂SO₄, filtered, and concentrated under reduced pressure. The residue was solubilized in a minimum of acetone and added dropwise to n-pentane. The resulting precipitate was triturated for 1 h, filtered, washed with n-pentane, and dried under reduced pressure for 12 h at 50° C. to afford N-[2-[2-chloro-3-[(R)-[1-[2-[(3S)-2,6-dioxo-3-piperidyl]-6-methyl-1-oxo-isoindoline-5-carbonyl]-4-piperidyl]-phenyl-methyl]phenoxy]ethyl]acetamide as a white powder (3.1 mg, 98.3% p., 17%, y.). m/z [M+H]⁺=671.3, ¹H NMR (500 MHz, DMSO-d₆): δ (ppm) 10.97 (s, 1H), 8.11-7.95 (m, 1H), 7.60 (br d, J=6.6 Hz, 1H), 7.48 (s, 1H), 7.34 (br s, 2H), 7.33-7.28 (m, 2H), 7.28-7.25 (m, 1H), 7.24-7.19 (m, 1H), 7.19-7.08 (m, 1H), 6.99 (s, 1H), 5.20-5.00 (m, 1H), 4.57-4.46 (m, 1H), 4.46-4.36 (m, 1H), 4.35-4.26 (m, 1H), 4.25-4.17 (m, 1H), 4.07-3.89 (m, 2H), 3.44-3.37 (m, 2H), 3.28-3.14 (m, 1H), 3.05-2.94 (m, 1H), 2.89 (br s, 1H), 2.83-2.70 (m, 1H), 2.64-2.59 (m, 1H), 2.58-2.55 (m, 1H), 2.44-2.36 (m, 1H), 2.25 (s, 3H), 2.04-1.92 (m, 1H), 1.80 (d, J=11.3 Hz, 3H), 1.71-1.03 (m, 4H).

### Example 58

### (3S)-3-[5-[4-[(R)-[3-[(1S,3S)-3-aminocyclopentoxy]-2-chloro-phenyl]-phenyl-methyl]piperidine-1-carbonyl]-6-methyl-1-oxo-isoindolin-2-yl]piperidine-2,6-dione hydrochloride

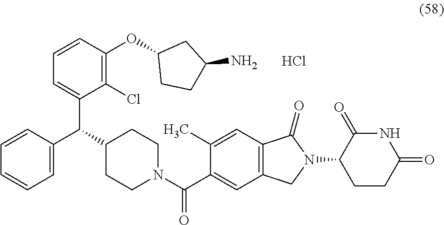 [SMILES: Cc1cc2c(cc1C(=O)N1CCC([C@H](c3ccccc3)c3cccc(O[C@H]4CC[C@H](N)C4)c3Cl)CC1)CN([C@H]1CCC(=O)NC1=O)C2=O ; Cl]

### Intermediate 58A: tert-butyl(4S)-5-amino-4-[5-[4-[(R)-[3-[(1S,3S)-3-(tert-butoxycarbonylamino)cyclopentoxy]-2-chloro-phenyl]-phenyl-methyl]piperidine-1-carbonyl]-6-methyl-1-oxo-isoindolin-2-yl]-5-oxo-pentanoate

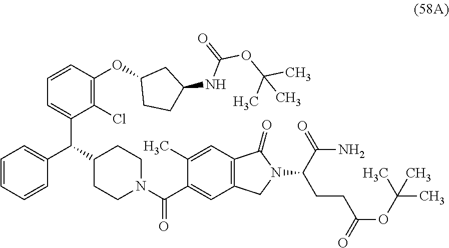 [SMILES: Cc1cc2c(cc1C(=O)N1CCC([C@H](c3ccccc3)c3cccc(O[C@H]4CC[C@H](NC(=O)OC(C)(C)C)C4)c3Cl)CC1)CN([C@@H](CCC(=O)OC(C)(C)C)C(N)=O)C2=O]

To a solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.227 mmol), triphenylphosphane (179 mg, 0.682 mmol), and tert-butyl N-[(1S,3R)-3-hydroxycyclopentyl]carbamate (141 mg, 0.682 mmol) in dry THF (1.5 mL) was added DIAD (134 μL, 0.682 mmol) at ambient temperature. The mixture was stirred at this temperature for 16 hours. The mixture was concentrated under reduced pressure. The residue was purified by flash chromatography on silica gel using a gradient of EtOAc/EtOH (3/1) (2% conc. NH₄OH) in n-heptane from 5 to 100% to afford (tert-butyl(4S)-5-amino-4-[5-[4-[(R)-[3-[(1S,3S)-3-(tert-butoxycarbonylamino)cyclopentoxy]-2-chloro-phenyl]-phenyl-methyl]piperidine-1-carbonyl]-6-methyl-1-oxo-isoindolin-2-yl]-5-oxo-pentanoate as a white solid (141 mg, 95% p., 70% y.). m/z [M+Na]⁺=865.4. [α]_{D}=+32°·mL·dm⁻¹·g⁻¹[20° C., Na, 589 nm, C=1.1 mg/mL, EtOH].

### Example 58

To a solution of tert-butyl(4S)-5-amino-4-[5-[4-[(R)-[3-[(1S,3S)-3-(tert-butoxycarbonylamino)cyclopentoxy]-2-chloro-phenyl]-phenyl-methyl]piperidine-1-carbonyl]-6-methyl-1-oxo-isoindolin-2-yl]-5-oxo-pentanoate (141 mg, 0.159 mmol) in dry acetonitrile (5.0 mL) was added 4-methylbenzenesulfonic acid hydrate (90.6 mg, 0.476 mmol). The mixture was stirred for 4 hours at 85° C. before concentrating under reduced pressure. The resulting mixture was purified by reverse-phase preparative HPLC (0-100% acetonitrile in water, 0.1% AcOH), by flash chromatography on silica gel (20-100% EtOAc/EtOH (3/1) in n-heptane, then 0-60% EtOH in EtOAc), and by reverse-phase preparative chromatography using a gradient of acetonitrile in water from 0% to 100% (0.1% AcOH). The mixture was solubilized in acetonitrile and a 1 M aqueous solution of HCl (50 μL) was added and was lyophilized to afford (3S)-3-[5-[4-[(R)-[3-[(1S,3S)-3-aminocyclopentoxy]-2-chloro-phenyl]-phenyl-methyl]piperidine-1-carbonyl]-6-methyl-1-oxo-isoindolin-2-yl]piperidine-2,6-dione hydrochloride as a white powder.

(18.7 mg, 100% p., 17% y.). m/z [M+H]⁺=669.4, ¹H NMR (600 MHz, DMSO-d₆): δ (ppm) 11.01-10.93 (m, 1H), 7.99-7.78 (m, 3H), 7.64-7.57 (m, 1H), 7.50-7.21 (m, 7H), 7.21-7.09 (m, 1H), 6.99-6.85 (m, 1H), 5.18-5.04 (m, 1H), 5.00-4.87 (m, 1H), 4.57-4.46 (m, 1H), 4.44-4.35 (m, 1H), 4.34-4.14 (m, 2H), 3.77-3.58 (m, 1H), 3.27-3.13 (m, 1H), 3.03-2.84 (m, 2H), 2.83-2.70 (m, 1H), 2.63-2.52 (m, 2H), 2.42-2.22 (m, 4H), 2.22-2.03 (m, 3H), 2.03-1.85 (m, 2H), 1.79-1.66 (m, 1H), 1.66-0.86 (m, 5H), [α]_{D}=−17°·mL·dm⁻¹·g⁻¹[20° C., Na, 589 nm, C=0.6 mg/mL, DMSO].

### Example 59

### (3S)-3-[5-[4-[(R)-[3-[(2S)-2-amino-3,3-difluoro-propoxy]-2-chloro-phenyl]-phenyl-methyl]piperidine-1-carbonyl]-6-methyl-1-oxo-isoindolin-2-yl]piperidine-2,6-dione

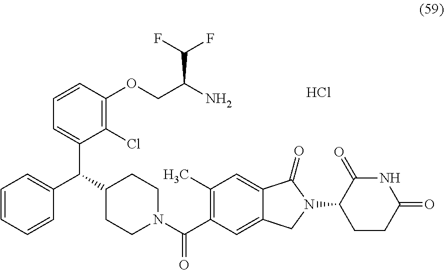 [SMILES: Cc1cc2c(cc1C(=O)N1CCC([C@H](c3ccccc3)c3cccc(OC[C@H](N)C(F)F)c3Cl)CC1)CN([C@H]1CCC(=O)NC1=O)C2=O ; Cl]

### Intermediate 59A: tert-butyl(4S)-5-amino-4-[5-[4-[(R)-[3-[(2S)-2-(tert-butoxycarbonylamino)-3,3-difluoro-propoxy]-2-chloro-phenyl]-phenyl-methyl]piperidine-1-carbonyl]-6-methyl-1-oxo-isoindolin-2-yl]-5-oxo-pentanoate

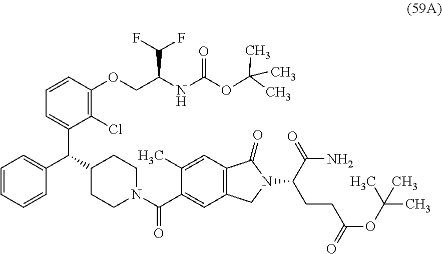 [SMILES: Cc1cc2c(cc1C(=O)N1CCC([C@H](c3ccccc3)c3cccc(OC[C@H](NC(=O)OC(C)(C)C)C(F)F)c3Cl)CC1)CN([C@@H](CCC(=O)OC(C)(C)C)C(N)=O)C2=O]

To a solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (120 mg, 0.182 mmol), triphenylphosphane (95.3 mg, 0.364 mmol), and tert-butyl N-[(2S)-1,1-difluoro-3-hydroxypropan-2-yl]carbamate (75.0 mg, 0.337 mmol) in THF (2.0 mL) was added DIAD (71.6 μL, 0.364 mmol) at ambient temperature. The mixture was stirred at this temperature for 16 h. The reaction mixture was concentrated under reduced pressure. The residue was purified by flash chromatography on silica gel using a gradient of EtOAc/EtOH (3/1) (2% conc. NH₄OH) in n-heptane from 5 to 100% to afford tert-butyl(4S)-5-amino-4-[5-[4-[(R)-[3-[(2S)-2-(tert-butoxycarbonylamino)-3,3-difluoro-propoxy]-2-chloro-phenyl]-phenyl-methyl]piperidine-1-carbonyl]-6-methyl-1-oxo-isoindolin-2-yl]-5-oxo-pentanoate as a white solid (113 mg, 78% p., 57% y.). m/z [M+Na]⁺=875.4.

### Example 59

To a solution of tert-butyl(4S)-5-amino-4-[5-[4-[(R)-[3-[(2S)-2-(tert-butoxycarbonylamino)-3,3-difluoro-propoxy]-2-chloro-phenyl]-phenyl-methyl]piperidine-1-carbonyl]-6-methyl-1-oxo-isoindolin-2-yl]-5-oxo-pentanoate (113 mg, 0.103 mmol) in dry acetonitrile (4.0 mL) was added 4-methylbenzenesulfonic acid hydrate (58.9 mg, 0.310 mmol). The mixture was stirred at 80° C. for 4 h, before concentrating under reduced pressure. The residue was purified by preparative HPLC using a gradient of acetonitrile in water from 0% to 100% (0.1% AcOH). The desired fractions were combined and concentrated under reduced pressure. The residue was partitioned between EtOAc and water. The organic layer was filtered over hydrophobic filter paper, and concentrated under reduced pressure. The resulting solid was purified by reverse-phase preparative chromatography using a gradient of acetonitrile in water from 0% to 100% (0.1% AcOH) to afford the product as a colorless gum after concentration under reduced pressure. The latter was solubilized in acetonitrile/water, and a 1 M aqueous solution of HCl (30.0 μL) was added. The mixture was lyophilized for 72 hours to afford ((3S)-3-[5-[4-[(R)-[3-[(2S)-2-amino-3,3-difluoro-propoxy]-2-chloro-phenyl]-phenyl-methyl]piperidine-1-carbonyl]-6-methyl-1-oxo-isoindolin-2-yl]piperidine-2,6-dione hydrochloride as a white solid (21.1 mg, 98% p., 28% y.). m/z [M+H]⁺679.4, ¹H NMR (600 MHz, DMSO-d₆): δ (ppm) 11.06-10.88 (m, 1H), 8.98-8.14 (m, 3H), 7.64-7.57 (m, 1H), 7.48-7.21 (m, 7H), 7.20-7.10 (m, 1H), 7.08-6.96 (m, 1H), 6.56-6.27 (m, 1H), 5.15-5.05 (m, 1H), 4.60-4.46 (m, 1H), 4.46-4.35 (m, 1H), 4.35-4.17 (m, 4H), 4.15-4.00 (m, 1H), 3.28-3.14 (m, 1H), 3.05-2.94 (m, 1H), 2.94-2.84 (m, 1H), 2.84-2.70 (m, 1H), 2.64-2.54 (m, 2H), 2.43-2.20 (m, 4H), 2.05-1.92 (m, 1H), 1.68-0.86 (m, 4H), [α]_{D}=−25°·mL·dm⁻¹·g⁻¹[20° C., Na, 589 nm, C=0.8 mg/mL, DMSO].

### Example 60

### (3S)-3-[5-[4-[(R)-[2-chloro-3-(2-methoxyethoxy)phenyl]-phenyl-methyl]piperidine-1-carbonyl]-6-methyl-1-oxo-isoindolin-2-yl]piperidine-2,6-dione

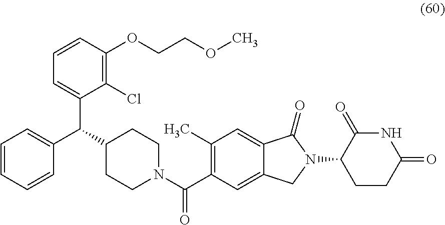 [SMILES: COCCOc1cccc([C@@H](c2ccccc2)C2CCN(C(=O)c3cc4c(cc3C)C(=O)N([C@H]3CCC(=O)NC3=O)C4)CC2)c1Cl]

### Intermediate 60A: tert-butyl(4S)-5-amino-4-[5-[4-[(R)-[2-chloro-3-(2-methoxyethoxy)-phenyl]phenyl-methyl]piperidine-1-carbonyl]-6-methyl-1-oxo-isoindolin-2-yl]-5-oxo-pentanoate

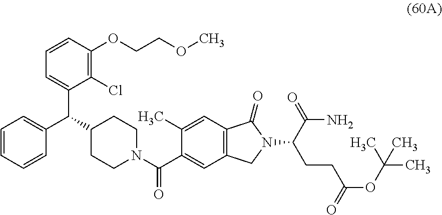 [SMILES: COCCOc1cccc([C@@H](c2ccccc2)C2CCN(C(=O)c3cc4c(cc3C)C(=O)N([C@@H](CCC(=O)OC(C)(C)C)C(N)=O)C4)CC2)c1Cl]

In a sealed vial, to a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.211 mmol) in dry THF (1.4 mL) were added successively 2-methoxyethanol (50.0 μL, 0.634 mmol), triphenylphosphane (166 mg, 0.634 mmol), and then DIAD (125 μL, 0.634 mmol). The reaction mixture was stirred at ambient temperature for 72 h. The reaction mixture was concentrated under reduced pressure. The crude was purified by flash chromatography on silica gel using a gradient of EtOAc/EtOH (3/1) in n-heptane from 5% to 100% to afford tert-butyl(4S)-5-amino-4-[5-[4-[(R)-[2-chloro-3-(2-methoxyethoxy)phenyl]-phenyl-methyl]piperidine-1-carbonyl]-6-methyl-1-oxo-isoindolin-2-yl]-5-oxo-pentanoate as a white solid (146 mg, 89% p., 85% y.). m/z [M+Na]⁺=740.3.

### Example 60

To a stirred solution of tert-butyl(4S)-5-amino-4-[5-[4-[(R)-[2-chloro-3-(2-methoxyethoxy)phenyl]-phenyl-methyl]piperidine-1-carbonyl]-6-methyl-1-oxo-isoindolin-2-yl]-5-oxo-pentanoate (145 mg, 0.202 mmol) in acetonitrile (2.0 mL) was added 4-methylbenzenesulfonic acid hydrate (192 mg, 1.01 mmol). The reaction mixture was stirred at 80° C. for 3 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure. The aqueous layer was basified with a saturated aqueous layer of NaHCO₃. The precipitate was filtered and dried under reduced pressure at 50° C. for 16 h to afford (3S)-3-[5-[4-[(R)-[2-chloro-3-(2-methoxyethoxy)phenyl]-phenyl-methyl]piperidine-1-carbonyl]-6-methyl-1-oxo-isoindolin-2-yl]piperidine-2,6-dione as a white powder (57.4 mg, 100% p., 44% y.). m/z [M+H]⁺=644.3, ¹H NMR (600 MHz, DMSO-d₆): δ (ppm) 10.97 (s, 1H), 7.66-7.57 (m, 1H), 7.52-7.08 (m, 8H), 7.02-6.84 (m, 1H), 5.15-5.03 (m, 1H), 4.61-4.35 (m, 2H), 4.34-3.98 (m, 4H), 3.72-3.60 (m, 2H), 3.32-3.27 (m, 3H), 3.27-3.13 (m, 1H), 3.05-2.83 (m, 2H), 2.82-2.70 (m, 1H), 2.66-2.51 (m, 2H), 2.45-2.30 (m, 2H), 2.24 (s, 2H), 2.03-1.88 (m, 1H), 1.70-0.83 (m, 4H).

### Example 61

### (3S)-3-[5-[4-[(R)-[3-(3-aminopropoxy)-2-chloro-phenyl]-phenyl-methyl]piperidine-1-carbonyl]-6-methyl-1-oxo-isoindolin-2-yl]piperidine-2,6-dione hydrochloride

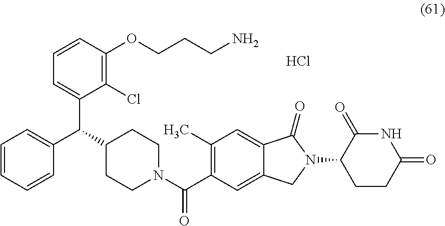 [SMILES: Cc1cc2c(cc1C(=O)N1CCC([C@H](c3ccccc3)c3cccc(OCCCN)c3Cl)CC1)CN([C@H]1CCC(=O)NC1=O)C2=O ; Cl]

### Intermediate 61A: tert-butyl(4S)-5-amino-4-[5-[4-[(R)-[3-[3-(tert-butoxycarbonylamino) propoxy]-2-chloro-phenyl]-phenyl-methyl]piperidine-1-carbonyl]-6-methyl-1-oxo-isoindolin-2-yl]-5-oxo-pentanoate

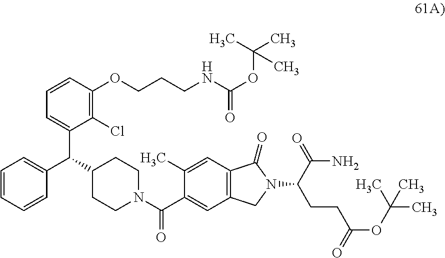 [SMILES: Cc1cc2c(cc1C(=O)N1CCC([C@H](c3ccccc3)c3cccc(OCCCNC(=O)OC(C)(C)C)c3Cl)CC1)CN([C@@H](CCC(=O)OC(C)(C)C)C(N)=O)C2=O]

To a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.211 mmol) and tert-butyl(3-hydroxypropyl) carbamate (111 μL, 0.634 mmol) in dry THF (1.4 mL) were added successively triphenylphosphane (166 mg, 0.634 mmol) and then DIAD (125 μL, 0.634 mmol). The reaction mixture was stirred at ambient temperature overnight. The reaction mixture was concentrated under reduced pressure. The crude was purified by flash chromatography on silica gel using a gradient of EtOAc/EtOH (3/1) (0.5% conc. NH₄OH) in n-heptane from 5% to 100% to afford tert-butyl(4S)-5-amino-4-[5-[4-[(R)-[3-[3-(tert-butoxycarbonylamino) propoxy]-2-chloro-phenyl]-phenyl-methyl]piperidine-1-carbonyl]-6-methyl-1-oxo-isoindolin-2-yl]-5-oxo-pentanoate as a white solid (131 mg, 99% p., 75% y.). m/z [M+H]⁺=817.3. [α]_{D}=+25°·mL·dm⁻¹·g⁻¹[20° C., Na, 589 nm, C=1.0 mg/mL, EtOH].

### Example 61

A vial was successively charged with 4-methylbenzenesulfonic acid hydrate (156 mg, 0.820 mmol) and tert-butyl(4S)-5-amino-4-[5-[4-[(R)-[3-[3-(tert-butoxycarbonylamino) propoxy]-2-chloro-phenyl]-phenyl-methyl]piperidine-1-carbonyl]-6-methyl-1-oxo-isoindolin-2-yl]-5-oxo-pentanoate (126 mg, 0.153 mmol) in acetonitrile (1.5 mL). The vial was sealed, and the mixture was stirred at 80° C. for 4 h. The mixture was cooled at ambient temperature. The reaction was quenched with the addition of water. A solution of EtOAc/isopropanol (3/1) and a saturated aqueous solution of NaHCO₃(until basic pH) were added. The organic layer was isolated, washed once with saturated aqueous solution of NaHCO₃, once with brine then filtered over hydrophobic paper and concentrated. The crude was purified by reverse-phase preparative chromatography using a gradient of acetonitrile in water from 0% to 60% (0.1% AcOH). The desired fractions were combined and a saturated aqueous solution of NaHCO₃ was added until basic pH. The mixture was extracted with EtOAc (2×). The organic layer was isolated, washed with brine and concentrated under reduced pressure. The residue was lyophilized (in acetonitrile/water+2 equivalents of 1 N aqueous solution of HCl) to afford ((3S)-3-[5-[4-[(R)-[3-(3-aminopropoxy)-2-chloro-phenyl]-phenyl-methyl]piperidine-1-carbonyl]-6-methyl-1-oxo-isoindolin-2-yl]piperidine-2,6-dione hydrochloride (7.5 mg, 96.8% p., 7% y.) as a white solid. m/z [M+H]⁺=643.3, ¹H NMR (500 MHz, DMSO-d₆): δ (ppm) 10.98 (s, 1H), 8.00-7.69 (m, 3H), 7.62 (br d, J=6.0 Hz, 1H), 7.51-7.05 (m, 8H), 7.03-6.70 (m, 1H), 5.25-4.96 (m, 1H), 4.76-3.77 (m, 6H), 3.28-3.15 (m, 1H), 3.09-2.70 (m, 5H), 2.68-2.53 (m, 2H), 2.47-2.18 (m, 4H), 2.13-1.84 (m, 3H), 1.71-0.69 (m, 4H), [α]_{D}=+23°·mL·dm⁻¹ g⁻¹[20° C., Na, 589 nm, C=1.1 mg/mL, DMSO].

### Example 62

### (3S)-3-[5-[4-[(R)-[3-[(2S)-2-amino-3-methyl-butoxy]-2-chloro-phenyl]-phenyl-methyl]piperidine-1-carbonyl]-6-methyl-1-oxo-isoindolin-2-yl]piperidine-2,6-dione

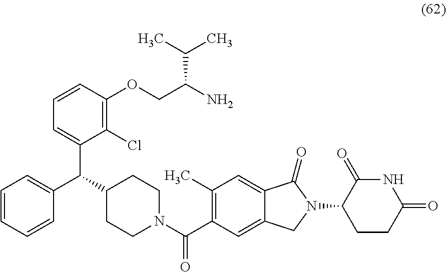 [SMILES: Cc1cc2c(cc1C(=O)N1CCC([C@H](c3ccccc3)c3cccc(OC[C@@H](N)C(C)C)c3Cl)CC1)CN([C@H]1CCC(=O)NC1=O)C2=O]

### Intermediate 62A: tert-butyl(4S)-5-amino-4-[5-[4-[(R)-[3-[(2S)-2-(tert-butoxycarbonylamino)-3-methyl-butoxy]-2-chloro-phenyl]-phenyl-methyl]piperidine-1-carbonyl]-6-methyl-1-oxo-isoindolin-2-yl]-5-oxo-pentanoate

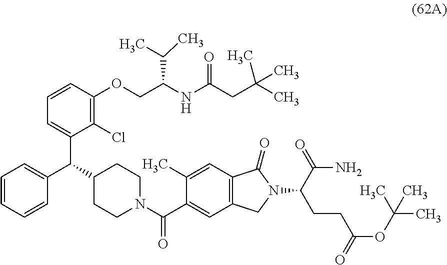 [SMILES: Cc1cc2c(cc1C(=O)N1CCC([C@H](c3ccccc3)c3cccc(OC[C@@H](NC(=O)CC(C)(C)C)C(C)C)c3Cl)CC1)CN([C@@H](CCC(=O)OC(C)(C)C)C(N)=O)C2=O]

To a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.211 mmol) and tert-butyl N-[(2S)-1-hydroxy-3-methylbutan-2-yl]carbamate (133 mg, 0.634 mmol) in dry THF (1.4 mL) were added successively triphenylphosphane (166 mg, 0.634 mmol) and then DIAD (125 μL, 0.634 mmol). The reaction mixture was stirred at ambient temperature overnight. The reaction mixture was concentrated under reduced pressure. The crude was purified by flash chromatography on silica gel using a gradient of EtOAc/EtOH (3/1) in n-heptane from 5% to 90% to afford tert-butyl(4S)-5-amino-4-[5-[4-[(R)-[3-[(2S)-2-(tert-butoxycarbonylamino)-3-methyl-butoxy]-2-chloro-phenyl]-phenyl-methyl]piperidine-1-carbonyl]-6-methyl-1-oxo-isoindolin-2-yl]-5-oxo-pentanoate as a white solid (127 mg, 90% p., 64% y.). m/z [M+H]⁺=845.3, ¹H NMR (600 MHz, DMSO-d₆): δ (ppm) 7.63-7.50 (m, 2H), 7.49-7.05 (m, 9H), 6.99-6.84 (m, 1H), 6.75-6.62 (m, 1H), 4.71 (dd, J=10.4, 4.1 Hz, 1H), 4.64-4.30 (m, 3H), 4.27-4.13 (m, 1H), 4.09-3.75 (m, 2H), 3.69-3.49 (m, 1H), 3.28-3.09 (m, 1H), 3.05-2.89 (m, 1H), 2.85-2.67 (m, 1H), 2.62-2.51 (m, 1H), 2.37-1.70 (m, 8H), 1.67-0.93 (m, 22H), 0.92-0.64 (m, 6H), [α]_{D}=+4°·mL·dm⁻¹ g⁻¹[20° C., Na, 589 nm, C=1.1 mg/mL, EtOH].

### Example 62

A vial was successively charged with 4-methylbenzenesulfonic acid hydrate (155 mg, 0.815 mmol) and tert-butyl(4S)-5-amino-4-[5-[4-[(R)-[3-[(2S)-2-(tert-butoxycarbonylamino)-3-methyl-butoxy]-2-chloro-phenyl]-phenyl-methyl]piperidine-1-carbonyl]-6-methyl-1-oxo-isoindolin-2-yl]-5-oxo-pentanoate (120 mg, 0.128 mmol) in acetonitrile (1.5 mL). The vial was sealed, and the mixture was stirred at 80° C. for 3.5 h. The mixture was cooled at ambient temperature. The reaction was quenched with the addition of water. A solution of EtOAc/isopropanol 3/1 and a saturated aqueous solution of NaHCO₃(until basic pH) were added. The organic layer was isolated, washed once with saturated aqueous solution of NaHCO₃, once with brine then filtered over hydrophobic paper, and concentrated. The crude was purified by reverse-phase preparative chromatography using a gradient of acetonitrile in water from 0% to 50% (0.1% AcOH). The desired fractions were collected and saturated aqueous solution of NaHCO₃ was added until basic pH. The mixture was extracted with EtOAc (2×). The organic layer was isolated, washed with brine and concentrated under reduced pressure. The residue was lyophilized (in acetonitrile/water+2 equivalents of 1 N aqueous solution of HCl) to afford (3S)-3-[5-[4-[(R)-[3-[(2S)-2-amino-3-methyl-butoxy]-2-chloro-phenyl]-phenyl-methyl]piperidine-1-carbonyl]-6-methyl-1-oxo-isoindolin-2-yl]piperidine-2,6-dione hydrochloride as a white solid (26.5 mg, 99.8% p., 29% y.). m/z [M+H]=671.3, ¹H NMR (500 MHz, DMSO-d₆): δ (ppm) 10.98 (br s, 1H), 8.05 (br s, 3H), 7.62 (br d, J=8.1 Hz, 1H), 7.46 (br d, J=9.8 Hz, 1H), 7.41-7.21 (m, 5H), 7.21-7.11 (m, 1H), 7.09-6.96 (m, 1H), 7.77-6.85 (m, 1H), 5.11 (br dd, J=13.1, 4.7 Hz, 1H), 4.52 (br s, 1H), 4.47-4.37 (m, 1H), 4.35-4.28 (m, 1H), 4.25-4.12 (m, 3H), 3.30-3.19 (m, 2H), 3.00 (br d, J=12.1 Hz, 1H), 2.96-2.85 (m, 1H), 2.83-2.73 (m, 1H), 2.59 (br d, J=13.9 Hz, 2H), 2.41-2.31 (m, 2H), 2.25 (br s, 2H), 2.14-2.05 (m, 1H), 2.04-1.96 (m, 1H), 1.67-1.06 (m, 4H), 1.05-0.96 (m, 6H), [α]_{D}=+27°·mL·dm⁻¹·g⁻¹[20° C., Na, 589 nm, C=1.1 mg/mL, DMSO].

### Example 63

### (3S)-3-[5-[4-[indolin-4-yl(phenyl)methyl]piperidine-1-carbonyl]-6-methyl-1-oxo-isoindolin-2-yl]piperidine-2,6-dione hydrochloride

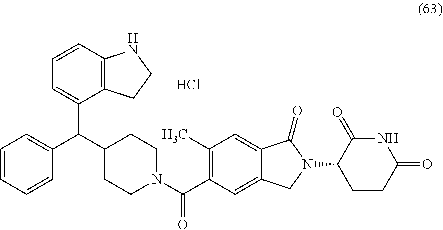 [SMILES: Cc1cc2c(cc1C(=O)N1CCC(C(c3ccccc3)c3cccc4c3CCN4)CC1)CN([C@H]1CCC(=O)NC1=O)C2=O ; Cl]

### Intermediate 63A: tert-butyl 4-bromoindoline-1-carboxylate

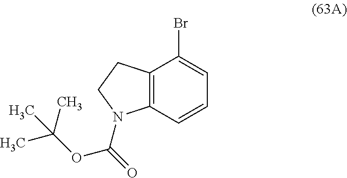 [SMILES: CC(C)(C)OC(=O)N1CCc2c(Br)cccc21]

In a round-bottom flask, to a stirred solution of 4-bromo-2,3-dihydro-1H-indole (650 mg, 3.12 mmol) in THF (6.2 mL) was added tert-butoxycarbonyl tert-butyl carbonate (842 mg, 3.74 mmol) followed by DMAP (38.1 mg, 0.312 mmol). The resulting mixture was stirred at ambient temperature overnight. The reaction was quenched with the addition of water. EtOAc was added to the mixture. The layers were separated, and the aqueous layer was extracted with EtOAc (2×). The combined organic layers were washed with brine, dried using a phase separator cartridge and concentrated under reduced pressure. The crude was purified by flash chromatography on silica gel using a gradient of EtOAc in n-heptane from 0% to 30%. The desired fractions were combined and concentrated under reduced pressure to afford tert-butyl 4-bromoindoline-1-carboxylate as a white solid (806 mg, 99% p., 86% y.). m/z [M-tBu+H]⁺=242.1, 1H NMR (400 MHz, DMSO-d₆): δ (ppm) 7.62 (s, 1H), 7.18-7.06 (m, 2H), 3.94 (dd, J=9.3, 8.1 Hz, 2H), 3.03 (t, J=8.7 Hz, 2H), 1.50 (s, 9H).

### Intermediate 63B: tert-butyl 4-(4,4,5,5-tetramethyl-1,3,2-dioxaborolan-2-yl) indoline-1-carboxylate

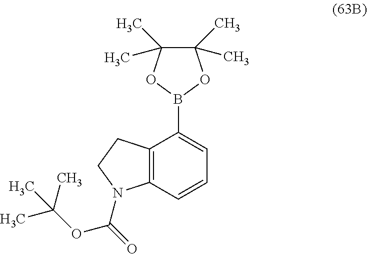 [SMILES: CC(C)(C)OC(=O)N1CCc2c(B3OC(C)(C)C(C)(C)O3)cccc21]

In a sealed vial, to a stirred solution of tert-butyl 4-bromoindoline-1-carboxylate (500 mg, 1.68 mmol) in dry 1,4-dioxane (9.0 mL) under argon were added successively B₂pin₂ (652 mg, 2.52 mmol) and KOAc (494 mg, 5.03 mmol). The suspension was degassed with argon for 5 min and Pd(dppf)Cl₂(123 mg, 0.168 mmol) was added. The mixture was stirred at 90° C. overnight. The suspension was filtered over glass fiber and rinsed with EtOAc. A saturated aqueous solution of NaHCO₃ was added and the aqueous layer was extracted with EtOAc (3×). The combined organic layers were washed with brine, dried using a phase separator cartridge, and concentrated under reduced pressure. The crude was purified by flash chromatography on silica gel using a gradient of EtOAc in n-heptane from 0% to 30%. The desired fractions were combined and concentrated under reduced pressure to afford tert-butyl 4-(4,4,5,5-tetramethyl-1,3,2-dioxaborolan-2-yl) indoline-1-carboxylate as a white gum (478 mg, 98%, 81% y.). m/z [M-tBu+H]⁺=290.2, ¹H NMR (400 MHz, DMSO-d₆): δ (ppm) 7.78 (s, 1H), 7.21 (dd, J=7.5, 1.2 Hz, 1H), 7.14 (t, J=7.7 Hz, 1H), 3.88 (dd, J=9.2, 8.2 Hz, 2H), 3.17 (t, J=8.7 Hz, 2H), 1.50 (s, 9H), 1.29 (s, 12H).

### Intermediate 63C: tert-butyl 4-[(1-benzyloxycarbonyl-4-piperidylidene)-phenyl-methyl]indoline-1-carboxylate

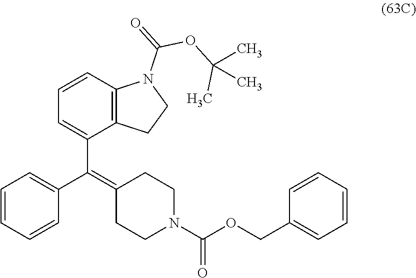 [SMILES: CC(C)(C)OC(=O)N1CCc2c(C(=C3CCN(C(=O)OCc4ccccc4)CC3)c3ccccc3)cccc21]

In a sealed vial, to a stirred solution of benzyl 4-[bromo (phenyl)methylene]piperidine-1-carboxylate (450 mg, 1.16 mmol) in 1,4-dioxane (8.1 mL) and water (2.7 mL) were added under argon tert-butyl 4-(4,4,5,5-tetramethyl-1,3,2-dioxaborolan-2-yl) indoline-1-carboxylate (483 mg, 1.40 mmol) and K₃PO₄ (502 mg, 2.33 mmol). The reaction mixture was degassed with argon for 5 min after which Pd(PPh3) 4 (137 mg, 0.116 mmol) was added. The reaction mixture was stirred at 90° C. for 4 h. EtOAc was added, and the layers were separated. The aqueous layer was extracted with EtOAc (2×). The combined organic layers were washed with brine, dried using a phase separator cartridge, and concentrated under reduced pressure. The crude was purified by flash chromatography on silica gel using a gradient of EtOAc in n-heptane from 0% to 40%. The desired fractions were combined and concentrated under reduced pressure to afford tert-butyl 4-[(1-benzyloxycarbonyl-4-piperidylidene)-phenyl-methyl]indoline-1-carboxylate as a white foam (559 mg, 98% p., 90% y.). m/z [M-Boc+2H]⁺=425.4, 1H NMR (400 MHz, DMSO-d₆): δ (ppm) 7.56 (s, 1H), 7.38-7.28 (m, 7H), 7.24-7.19 (m, 1H), 7.17-7.10 (m, 3H), 6.76 (dd, J=7.7, 1.0 Hz, 1H), 5.09 (s, 2H), 3.82 (s, 2H), 3.45 (s, 4H), 2.82 (s, 1H), 2.59 (s, 1H), 2.31 (s, 2H), 2.11 (d, J=6.2 Hz, 2H), 1.48 (s, 9H).

### Intermediate 63D: tert-butyl 4-[phenyl(4-piperidyl)methyl]indoline-1-carboxylate

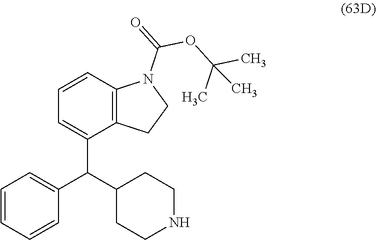 [SMILES: CC(C)(C)OC(=O)N1CCc2c(C(c3ccccc3)C3CCNCC3)cccc21]

In a sealed vial, to a stirred solution of tert-butyl 4-[(1-benzyloxycarbonyl-4-piperidylidene)-phenyl-methyl]indoline-1-carboxylate (300 mg, 0.572 mmol) in EtOAc (4.1 mL) under argon was added AcOH (32.7 μL, 0.572 mmol). The suspension was degassed with argon for 5 min and palladium on carbon (10% w/w, 304 mg, 0.286 mmol) and triethylsilane (554 μL, 3.43 mmol) were added. The mixture was stirred at 70° C. for 5 h. The suspension was filtered over glass fiber and rinsed with EtOAc. The filtrate was basified with a saturated aqueous solution of NaHCO₃ and EtOAc was added. The layers were separated, and the aqueous layer was extracted with EtOAc (2×). The combined organic layers were washed with brine, dried using a phase separator cartridge and concentrated under reduced pressure to afford tert-butyl 4-[phenyl(4-piperidyl)methyl]indoline-1-carboxylate as a pale brown oil (495 mg, 50% p., quant. yield)). m/z [M+H]⁺=393.4, ¹H NMR (400 MHz, DMSO-d₆): δ (ppm) 7.47 (s, 1H), 7.34-7.21 (m, 4H), 7.19-7.06 (m, 3H), 5.17 (s, 3H), 3.87 (pd, J=10.9, 6.8 Hz, 2H), 3.52 (d, J=11.0 Hz, 1H), 3.21-3.12 (m, 1H), 2.93 (dd, J=18.8, 9.5 Hz, 3H), 2.50-2.42 (m, 4H), 2.29 (q, J=11.2 Hz, 1H), 1.47 (s, 9H).

### Intermediate 63E: tert-butyl 4-[1-[2-[(1S)-4-tert-butoxy-1-carbamoyl-4-oxo-butyl]-6-methyl-1-oxo-isoindoline-5-carbonyl]-4-piperidyl]-phenyl-methyl]indoline-1-carboxylate

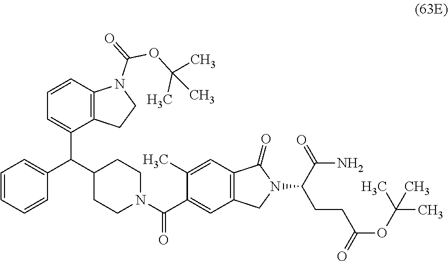 [SMILES: Cc1cc2c(cc1C(=O)N1CCC(C(c3ccccc3)c3cccc4c3CCN4C(=O)OC(C)(C)C)CC1)CN([C@@H](CCC(=O)OC(C)(C)C)C(N)=O)C2=O]

In a round-bottom flask, to a solution of tert-butyl 4-[phenyl(4-piperidyl)methyl]indoline-1-carboxylate (490 mg, 0.624 mmol) in DMF (4.0 mL), were added 2-[(1S)-4-tert-butoxy-1-carbamoyl-4-oxo-butyl]-6-methyl-1-oxo-isoindoline-5-carboxylic acid (235 mg, 0.624 mmol), DIPEA (327 μL, 1.87 mmol), and HATU (726 mg, 1.87 mmol). The mixture was stirred at ambient temperature for 4 h. The reaction was quenched with the addition of water. The mixture was basified with a saturated aqueous solution of NaHCO₃. EtOAc and then FastWorX® powder were added. The mixture was concentrated under reduced pressure and filtered through a dry-loading cartridge. The cartridge was rinsed with water. The crude was purified by flash chromatography on silica gel using a gradient of EtOAc/EtOH (3/1) in n-heptane from 5% to 100% to afford tert-butyl 4-[1-[2-[(1S)-4-tert-butoxy-1-carbamoyl-4-oxo-butyl]-6-methyl-1-oxo-isoindoline-5-carbonyl]-4-piperidyl]-phenyl-methyl]indoline-1-carboxylate as a pale yellow foam (439 mg, 99% p., 93% y.). m/z [M+Na]⁺=773.4.

### Example 63

To a stirred solution of tert-butyl 4-[1-[2-[(1S)-4-tert-butoxy-1-carbamoyl-4-oxo-butyl]-6-methyl-1-oxo-isoindoline-5-carbonyl]-4-piperidyl]-phenyl-methyl]indoline-1-carboxylate (130 mg, 0.173 mmol) in acetonitrile (1.8 mL) was added 4-methylbenzenesulfonic acid hydrate (200 mg, 1.05 mmol). The reaction mixture was stirred at 80° C. for 3 h. The reaction mixture was cooled to ambient temperature. The reaction mixture was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure. The solid was solubilized in 1,4-dioxane (2.6 mL) and a 4 M solution of HCl in 1,4-dioxane (260 μL) was added. The reaction mixture was stirred at ambient temperature for 30 min and Et₂O (26 mL) was added. After 1 h, the precipitate was filtered, washed with Et₂O, and dried under reduced pressure overnight to afford (3S)-3-[5-[4-[indolin-4-yl(phenyl)methyl]piperidine-1-carbonyl]-6-methyl-1-oxo-isoindolin-2-yl]piperidine-2,6-dione hydrochloride as a white powder (25.4 mg, 96.3% p., 23% y.). m/z [M+H]⁺=577.3, ¹H NMR (500 MHz, DMSO-d₆): δ (ppm) 11.15-10.78 (m, 1H), 10.84-8.99 (m, 1H), 7.62 (br s, 1H), 7.54-7.22 (m, 7H), 7.21-7.10 (m, 1H), 7.09-6.90 (m, 1H), 5.24-5.02 (m, 1H), 4.67-4.16 (m, 3H), 3.78-3.58 (m, 3H), 3.31-3.15 (m, 2H), 3.09-2.85 (m, 3H), 2.85-2.70 (m, 1H), 2.66-2.53 (m, 2H), 2.45-2.18 (m, 4H), 2.06-1.94 (m, 1H), 1.72-0.91 (m, 4H).

### Example 64

### (3S)-3-[5-[4-[(R)-[2-chloro-3-[2-[(3S)-pyrrolidin-3-yl]ethoxy]phenyl]-phenyl-methyl]piperidine-1-carbonyl]-6-methyl-1-oxo-isoindolin-2-yl]piperidine-2,6-dione 4-methylbenzenesulfonic acid

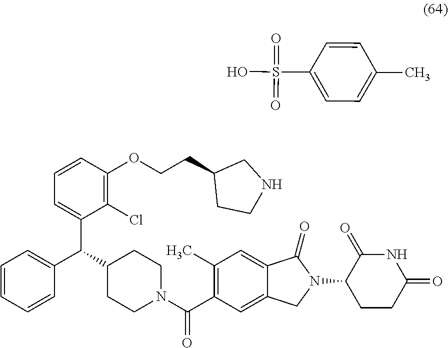 [SMILES: Cc1ccc(S(=O)(=O)O)cc1 ; Cc1cc2c(cc1C(=O)N1CCC([C@H](c3ccccc3)c3cccc(OCC[C@@H]4CCNC4)c3Cl)CC1)CN([C@H]1CCC(=O)NC1=O)C2=O]

### Intermediate 64A: tert-butyl(3S)-3-[2-[3-[(R)-[1-[2-[(1S)-4-tert-butoxy-1-carbamoyl-4-oxo-butyl]-6-methyl-1-oxo-isoindoline-5-carbonyl]-4-piperidyl]-phenyl-methyl]-2-chloro-phenoxy]ethyl]pyrrolidine-1-carboxylate

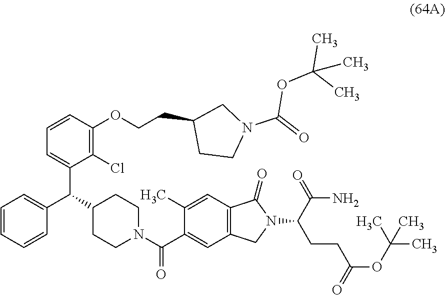 [SMILES: Cc1cc2c(cc1C(=O)N1CCC([C@H](c3ccccc3)c3cccc(OCC[C@@H]4CCN(C(=O)OC(C)(C)C)C4)c3Cl)CC1)CN([C@@H](CCC(=O)OC(C)(C)C)C(N)=O)C2=O]

To a solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.227 mmol), triphenylphosphane (179 mg, 0.682 mmol), and tert-butyl(3S)-3-(2-hydroxyethyl)pyrrolidine-1-carboxylate (154 mg, 0.682 mmol) in THF (1.5 mL) was added DIAD (134 μL, 0.682 mmol) at ambient temperature. The reaction mixture was stirred for 16 h. The reaction mixture was concentrated under reduced pressure. The crude was purified by flash chromatography on silica gel using a gradient of EtOAc/EtOH (3/1) (2% conc. NH₄OH) in cyclohexane from 5% to 100% to afford tert-butyl(3S)-3-[2-[3-[(R)-[1-[2-[(1S)-4-tert-butoxy-1-carbamoyl-4-oxo-butyl]-6-methyl-1-oxo-isoindoline-5-carbonyl]-4-piperidyl]-phenyl-methyl]-2-chloro-phenoxy]ethyl]pyrrolidine-1-carboxylate as a white solid (159 mg, 0.169 mmol, 91% p., 80% y.). m/z [M+Na]⁺=879.5. [α]_{D}=−7°·mL·dm⁻¹ g⁻¹[20° C., Na lamp, 589 nm, C=1.5 mg/mL, EtOH].

### Example 64

To a solution of tert-butyl(3S)-3-[2-[3-[(R)-[1-[2-[(1S)-4-tert-butoxy-1-carbamoyl-4-oxo-butyl]-6-methyl-1-oxo-isoindoline-5-carbonyl]-4-piperidyl]-phenyl-methyl]-2-chloro-phenoxy]ethyl]pyrrolidine-1-carboxylate (162 mg, 0.188 mmol) in dry acetonitrile (5.0 mL) was added 4-methylbenzenesulfonic acid hydrate (107 mg, 0.565 mmol). The mixture was stirred at 80° C. for 4 hours. The mixture was partitioned between EtOAc/iPrOH (3/1) and a saturated aqueous solution of NaHCO₃. Layers were separated and the organic layer was dried over hydrophobic paper and concentrated under reduced pressure. The crude was purified by reverse-phase preparative chromatography using a gradient of acetonitrile in water from 0% to 100% (0.1% AcOH). The desired fractions were combined and concentrated. The mixture was solubilized in an acetonitrile/water mixture, and the solution was freeze dried to afford (3S)-3-[5-[4-[(R)-[2-chloro-3-[2-[(3S)-pyrrolidin-3-yl]ethoxy]phenyl]-phenyl-methyl]piperidine-1-carbonyl]-6-methyl-1-oxo-isoindolin-2-yl]piperidine-2,6-dione 4-methylbenzenesulfonic acid as a white solid (16.3 mg, 95.0% p., 10% y.).m/z [M+H]⁺=683.4, ¹H NMR (600 MHz, DMSO-d₆): δ (ppm) 11.70-10.36 (m, 1H), 9.59-8.51 (m, 1H), 8.49-7.82 (m, 1H), 7.61 (br d, J=6.6 Hz, 1H), 7.46-7.38 (m, 1H), 7.38-7.35 (m, 1H), 7.34-7.31 (m, 2H), 7.31-7.28 (m, 1H), 7.28-7.21 (m, 2H), 7.21-7.12 (m, 1H), 7.00-6.85 (m, 1H), 5.17-5.00 (m, 1H), 4.58-4.46 (m, 1H), 4.46-4.36 (m, 1H), 4.35-4.25 (m, 1H), 4.23-4.14 (m, 1H), 4.13-3.91 (m, 2H), 3.39-3.34 (m, 1H), 3.25-3.20 (m, 1H), 3.20-3.15 (m, 1H), 3.10-3.02 (m, 1H), 3.02-2.93 (m, 1H), 2.93-2.83 (m, 1H), 2.82-2.68 (m, 2H), 2.66-2.53 (m, 2H), 2.44-2.29 (m, 3H), 2.28-2.21 (m, 2H), 2.13-2.04 (m, 1H), 2.03-1.93 (m, 1H), 1.89-1.78 (m, 2H), 1.69-0.76 (m, 5H), [α]_{D}=+33°·mL·dm⁻¹ g⁻¹[20° C., Na lamp, 589 nm, C=0.6 mg/mL, DMSO].

### Example 65

### (3S)-3-[5-[4-[(R)-[2-chloro-3-(methylamino)phenyl]-phenyl-methyl]piperidine-1-carbonyl]-6-methyl-1-oxo-isoindolin-2-yl]piperidine-2,6-dione

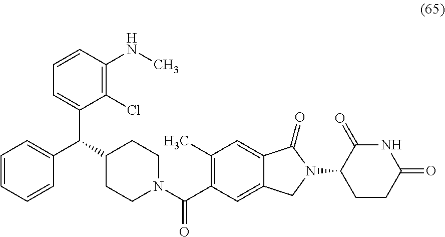 [SMILES: CNc1cccc([C@@H](c2ccccc2)C2CCN(C(=O)c3cc4c(cc3C)C(=O)N([C@H]3CCC(=O)NC3=O)C4)CC2)c1Cl]

### Intermediate 65A: benzyl 4-[(R)-[2-chloro-3-(methylamino)phenyl]-phenyl-methyl]piperidine-1-carboxylate

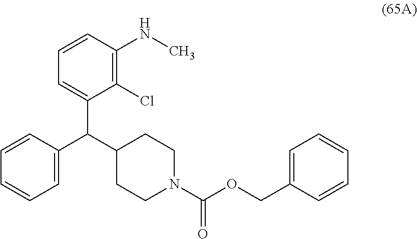 [SMILES: CNc1cccc(C(c2ccccc2)C2CCN(C(=O)OCc3ccccc3)CC2)c1Cl]

In a sealed vial, to a stirred solution of benzyl 4-[(R)-(3-bromo-2-chloro-phenyl)-phenyl-methyl]piperidine-1-carboxylate (300 mg, 0.571 mmol) in tert-butanol (2.9 mL) under argon were added successively t-BuONa (82.4 mg, 0.857 mmol) and 2 M methanamine (5.7 mL, 11.4 mmol) in THF. Brettphos-Pd-G3 (27.3 mg, 0.0286 mmol) and dicyclohexyl-[3,6-dimethoxy-2-(2,4,6-triisopropylphenyl)phenyl]phosphane (15.6 mg, 0.0286 mmol) were then added. The vial was sealed and heated at 80° C. for 2 h. The reaction mixture was cooled to ambient temperature. The reaction was quenched by addition of water and EtOAc. The aqueous layer was extracted with EtOAc (2×). The combined organic layers were washed with brine and dried with Na₂SO₄. The crude was purified by flash chromatography on silica gel using a gradient of EtOAc in cyclohexane from 0% to 50% to afford (benzyl 4-[(R)-[2-chloro-3-(methylamino)phenyl]-phenyl-methyl]piperidine-1-carboxylate (192 mg, 94% p., 70% y.). m/z [M+H]⁺=449.1, 1H NMR (400 MHz, DMSO-d₆): δ (ppm) 7.39-7.22 (m, 9H), 7.19-7.10 (m, 2H), 6.88 (dd, J=7.9, 1.4 Hz, 1H), 6.43 (dd, J=8.0, 1.4 Hz, 1H), 5.42 (q, J=4.9 Hz, 1H), 5.05 (s, 2H), 4.09 (d, J=11.2 Hz, 1H), 3.95 (d, J=13.2 Hz, 2H), 2.77 (s, 2H), 2.71 (d, J=4.9 Hz, 3H), 2.41 (d, J=11.4 Hz, 1H), 1.53 (d, J=13.1 Hz, 1H), 1.36 (d, J=13.6 Hz, 1H), 0.99 (dq, J=26.8, 12.5 Hz, 2H).

### Intermediate 65B: 2-chloro-N-methyl-3-[(R)-phenyl(4-piperidyl)methyl]aniline

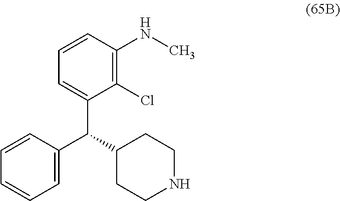 [SMILES: CNc1cccc([C@@H](c2ccccc2)C2CCNCC2)c1Cl]

To a stirred solution of benzyl 4-[(R)-[2-chloro-3-(methylamino)phenyl]-phenyl-methyl]piperidine-1-carboxylate (192 mg, 0.402 mmol) in dry acetonitrile (4.0 mL) at ambient temperature under nitrogen was added iodo (trimethyl) silane (172 μL, 1.21 mmol) dropwise. The reaction mixture was stirred at ambient temperature for 1 h. The mixture was cooled to 0° C. and MeOH (195 μL, 4.82 mmol) was added dropwise. The mixture was stirred at ambient temperature for 1 h and was concentrated under reduced pressure. Et₂O was added and the resulting mixture was stirred at ambient temperature for 1 h. The solid was filtered, washed with Et₂O, and dried under reduced pressure. The solid was purified by reverse-phase preparative chromatography using a gradient of acetonitrile in water from 0% to 100% (0.1% AcOH). Acetonitrile was evaporated and a saturated aqueous solution of NaHCO₃ was added. The aqueous layer was extracted with DCM. The combined organic layers were washed with brine, dried over Na₂SO₄, filtered, and concentrated under reduced pressure to afford 2-chloro-N-methyl-3-[(R)-phenyl(4-piperidyl)methyl]aniline as a white foam (69.0 mg, 98% p., 53% y.). m/z [M+H]⁺=315.3.

### Intermediate 65C: tert-butyl(4S)-5-amino-4-[5-[4-[(R)-[2-chloro-3-(methylamino) phenyl]-phenyl-methyl]piperidine-1-carbonyl]-6-methyl-1-oxo-isoindolin-2-yl]-5-oxo-pentanoate

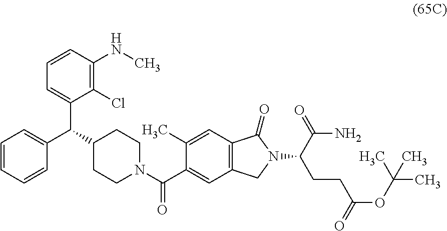 [SMILES: CNc1cccc([C@@H](c2ccccc2)C2CCN(C(=O)c3cc4c(cc3C)C(=O)N([C@@H](CCC(=O)OC(C)(C)C)C(N)=O)C4)CC2)c1Cl]

DIPEA (191 μL, 1.10 mmol) and a solution of T3P in DCM (53% w/w, 137 μL, 0.438 mmol) were added to a mixture of 2-chloro-N-methyl-3-[(R)-phenyl(4-piperidyl)methyl]aniline (69.0 mg, 0.219 mmol) and 2-[(1S)-4-tert-butoxy-1-carbamoyl-4-oxo-butyl]-6-methyl-1-oxo-isoindoline-5-carboxylic acid (90.7 mg, 0.241 mmol) in DCM (2.2 mL). The reaction mixture was stirred at ambient temperature for 2 h. The reaction was quenched with the addition of water. DCM was added to the mixture. The phases were separated and the aqueous phase extracted with DCM. Combined organic phases were washed with NaHCO₃ and brine, dried using a phase separator, and concentrated under reduced pressure to afford tert-butyl(4S)-5-amino-4-[5-[4-[(R)-[2-chloro-3-(methylamino)phenyl]-phenyl-methyl]piperidine-1-carbonyl]-6-methyl-1-oxo-isoindolin-2-yl]-5-oxo-pentanoate as a light brown oil (115 mg, 96% p., 75% y.). m/z [M+H]⁺=673.5.

### Example 65

In a sealed vial, to a solution of tert-butyl(4S)-5-amino-4-[5-[4-[(R)-[2-chloro-3-(methylamino)phenyl]-phenyl-methyl]piperidine-1-carbonyl]-6-methyl-1-oxo-isoindolin-2-yl]-5-oxo-pentanoate (115 mg, 0.171 mmol) in acetonitrile (1.0 mL) was added 4-methylbenzenesulfonic acid hydrate (162 mg, 0.854 mmol). The solution was stirred at 90° C. for 1 h. The mixture was diluted with EtOAc and washed with a saturated aqueous solution of NaHCO₃ and brine. The organic layer was dried over Na₂SO₄, filtered, and concentrated under reduced pressure. The crude was purified by reverse-phase preparative chromatography using a gradient of acetonitrile in water from 0% to 100% (0.1% AcOH). The desired fractions were combined and concentrated under reduced pressure until evaporation of most of the acetonitrile. A saturated aqueous solution of NaHCO₃ was added. The resulting suspension was filtered and dried under at 50° C. under reduced pressure overnight to afford (3S)-3-[5-[4-[(R)-[2-chloro-3-(methylamino) phenyl]-phenyl-methyl]piperidine-1-carbonyl]-6-methyl-1-oxo-isoindolin-2-yl]piperidine-2,6-dione as a white powder (29.4 mg, 95.3% p., 27% y.). m/z [M+H]⁺=599.4, ¹H NMR (600 MHz, DMSO-d₆) δ 10.97 (s, 1H), 7.65-7.55 (m, 1H), 7.49-7.19 (m, 5H), 7.19-7.07 (m, 2H), 6.97-6.82 (m, 1H), 6.48-6.33 (m, 1H), 5.48-5.35 (m, 1H), 5.14-5.02 (m, 1H), 4.55-4.46 (m, 1H), 4.46-4.22 (m, 2H), 4.18-4.06 (m, 1H), 3.27-3.13 (m, 1H), 3.04-2.64 (m, 6H), 2.63-2.55 (m, 1H), 2.53-2.50 (m, 1H), 2.44-2.17 (m, 4H), 2.05-1.89 (m, 1H), 1.73-0.88 (m, 4H).

### Example 66

### (3S)-3-[5-[4-[(R)-[3-[(2S)-2-aminopropoxy]-2-chloro-phenyl]-phenyl-methyl]piperidine-1-carbonyl]-6-methyl-1-oxo-isoindolin-2-yl]piperidine-2,6-dione hydrochloride

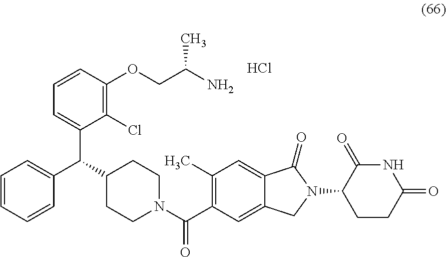 [SMILES: Cc1cc2c(cc1C(=O)N1CCC([C@H](c3ccccc3)c3cccc(OC[C@H](C)N)c3Cl)CC1)CN([C@H]1CCC(=O)NC1=O)C2=O ; Cl]

### Intermediate 66A: tert-butyl(4S)-5-amino-4-[5-[4-[(R)-[3-[(2S)-2-(tert-butoxycarbonylamino) propoxy]-2-chloro-phenyl]-phenyl-methyl]piperidine-1-carbonyl]-6-methyl-1-oxo-isoindolin-2-yl]-5-oxo-pentanoate

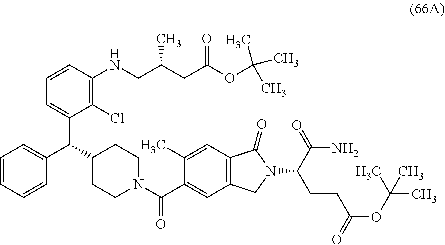 [SMILES: Cc1cc2c(cc1C(=O)N1CCC([C@H](c3ccccc3)c3cccc(NC[C@H](C)CC(=O)OC(C)(C)C)c3Cl)CC1)CN([C@@H](CCC(=O)OC(C)(C)C)C(N)=O)C2=O]

To a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.227 mmol) in dry THF (1.6 mL) were added successively triphenylphosphane (179 mg, 0.682 mmol), tert-butyl [(2S)-1-hydroxypropan-2-yl]carbamate (122 mg, 0.682 mmol), and then DIAD (134 μL, 0.682 mmol). The reaction mixture was stirred at ambient temperature for 72 h. The reaction mixture was concentrated under reduced pressure. The crude was purified by flash chromatography on silica gel using a gradient of EtOAc/EtOH (3/1) in n-heptane from 5% to 100% to afford tert-butyl(4S)-5-amino-4-[5-[4-[(R)-[3-[(2S)-2-(tert-butoxycarbonylamino) propoxy]-2-chloro-phenyl]-phenyl-methyl]piperidine-1-carbonyl]-6-methyl-1-oxo-isoindolin-2-yl]-5-oxo-pentanoate as a white solid (151 mg, 96% p., 78% y.). m/z [M-Boc+2H]⁺=717.4. [α]_{D}=+3°·mL·dm⁻¹·g⁻¹[20° C., Na lamp, 589 nm, C=2.0 mg/mL, EtOH].

### Example 66

In a sealed vial, to a solution of tert-butyl(4S)-5-amino-4-[5-[4-[(R)-[3-[(2S)-2-(tert-butoxycarbonylamino) propoxy]-2-chloro-phenyl]-phenyl-methyl]piperidine-1-carbonyl]-6-methyl-1-oxo-isoindolin-2-yl]-5-oxo-pentanoate (150 mg, 0.176 mmol) in acetonitrile (3.0 mL) was added 4-methylbenzenesulfonic acid hydrate (168 mg, 0.881 mmol). The solution was stirred at 90° C. for 4.5 h, then at ambient temperature overnight and 90° C. for 18 h. The mixture was diluted with EtOAc and washed with a saturated aqueous solution of NaHCO₃ and brine. The organic layer was dried over Na₂SO₄, filtered, and concentrated under reduced pressure. The crude was purified by reverse-phase preparative chromatography using a gradient of acetonitrile in water from 0% to 100% (0.1% AcOH). The desired fractions were combined and concentrated under reduced pressure until evaporation of most of the acetonitrile. A saturated aqueous solution of NaHCO₃ was added. The resulting suspension was extracted with EtOAc (2×). The organic layer was washed with brine, dried over Na₂SO₄, and filtered under reduced pressure. The residue was solubilized in 1,4-dioxane (3.0 mL) and a 4 M solution of HCl in 1,4-dioxane (500 μL) was added. The mixture was stirred at ambient temperature for 10 min. The resulting suspension was filtered and dried at 50° C. under reduced pressure for 96 hours to afford (3S)-3-[5-[4-[(R)-[3-[(2S)-2-aminopropoxy]-2-chloro-phenyl]-phenyl-methyl]piperidine-1-carbonyl]-6-methyl-1-oxo-isoindolin-2-yl]piperidine-2,6-dione hydrochloride as a white powder (40.8 mg, 100% p., 34% y.). m/z [M+H]⁺=643.4, ¹H NMR (600 MHz, DMSO-d₆): δ (ppm) 11.09-10.84 (m, 1H), 8.06-7.75 (m, 3H), 7.61 (br d, J=8.4 Hz, 1H), 7.49-7.20 (m, 7H), 7.20-7.08 (m, 1H), 7.04-6.91 (m, 1H), 5.14-5.04 (m, 1H), 4.58-4.47 (m, 1H), 4.46-4.36 (m, 1H), 4.35-4.26 (m, 1H), 4.25-4.17 (m, 1H), 4.10-3.98 (m, 2H), 3.64-3.54 (m, 1H), 3.27-3.15 (m, 1H), 3.05-2.94 (m, 1H), 2.94-2.85 (m, 1H), 2.84-2.70 (m, 1H), 2.64-2.53 (m, 2H), 2.42-2.22 (m, 4H), 2.06-1.92 (m, 1H), 1.67-0.88 (m, 7H), [α]_{D}=+11°·mL·dm⁻¹ g⁻¹[20° C., Na lamp, 589 nm, C=1.1 mg/mL, DMSO].

### Example 67

### (3S)-3-[5-[4-[(R)-[3-[(2S)-3-amino-2-fluoro-propoxy]-2-chloro-phenyl]-phenyl-methyl]piperidine-1-carbonyl]-6-methyl-1-oxo-isoindolin-2-yl]piperidine-2,6-dione hydrochloride

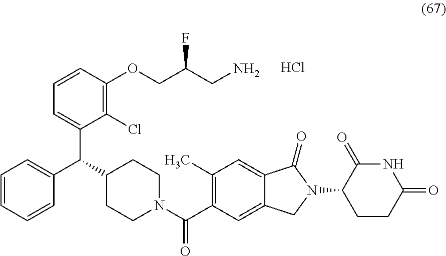 [SMILES: Cc1cc2c(cc1C(=O)N1CCC([C@H](c3ccccc3)c3cccc(OC[C@@H](F)CN)c3Cl)CC1)CN([C@H]1CCC(=O)NC1=O)C2=O ; Cl]

### Intermediate 67A: tert-butyl(4S)-5-amino-4-[5-[4-[(R)-[3-[(2S)-3-(tert-butoxycarbonylamino)-2-fluoro-propoxy]-2-chloro-phenyl]-phenyl-methyl]piperidine-1-carbonyl]-6-methyl-1-oxo-isoindolin-2-yl]-5-oxo-pentanoate

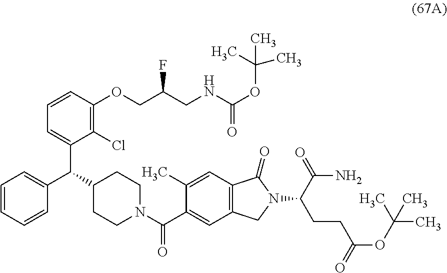 [SMILES: Cc1cc2c(cc1C(=O)N1CCC([C@H](c3ccccc3)c3cccc(OC[C@@H](F)CNC(=O)OC(C)(C)C)c3Cl)CC1)CN([C@@H](CCC(=O)OC(C)(C)C)C(N)=O)C2=O]

To a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.227 mmol) in dry THF (1.6 mL) were added successively triphenylphosphane (179 mg, 0.682 mmol), tert-butyl N-[(2S)-2-fluoro-3-hydroxypropyl]carbamate (139 mg, 0.682 mmol), and then DIAD (134 μL, 0.682 mmol). The reaction mixture was stirred at ambient temperature overnight. The reaction mixture was concentrated under reduced pressure. The crude was purified by flash chromatography on silica gel using a gradient of EtOAc/EtOH (3/1) in n-heptane from 5% to 100% to afford tert-butyl(4S)-5-amino-4-[5-[4-[(R)-[3-[(2S)-3-(tert-butoxycarbonylamino)-2-fluoro-propoxy]-2-chloro-phenyl]-phenyl-methyl]piperidine-1-carbonyl]-6-methyl-1-oxo-isoindolin-2-yl]-5-oxo-pentanoate as a white solid (119 mg, 88% p., 55% y.). m/z [M-Boc+2H]⁺=735.4. [α]_{D}=+21°·mL·dm⁻¹·g⁻¹[20° C., Na lamp, 589 nm, C=2.4 mg/mL, EtOH].

### Example 67

To a stirred solution of tert-butyl(4S)-5-amino-4-[5-[4-[(R)-[3-[(2S)-3-(tert-butoxycarbonylamino)-2-fluoro-propoxy]-2-chloro-phenyl]-phenyl-methyl]piperidine-1-carbonyl]-6-methyl-1-oxo-isoindolin-2-yl]-5-oxo-pentanoate (119 mg, 0.125 mmol) in acetonitrile (1.3 mL) was added 4-methylbenzenesulfonic acid hydrate (145 mg, 0.761 mmol). The reaction mixture was stirred at 80° C. for 3 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH in solvents). The desired fractions were concentrated under reduced pressure. The solid was solubilized in 1,4-dioxane (2.6 mL) and a 4 M solution of HCl in 1,4-dioxane (260 μL) was added. The reaction mixture was stirred at ambient temperature for 30 min and Et₂O (26 mL) was added. After 1 h, the precipitate was filtered, washed with Et₂O and dried under reduced pressure overnight to afford (3S)-3-[5-[4-[(R)-[3-[(2S)-3-amino-2-fluoro-propoxy]-2-chloro-phenyl]-phenyl-methyl]piperidine-1-carbonyl]-6-methyl-1-oxo-isoindolin-2-yl]piperidine-2,6-dione hydrochloride as a white powder (49.0 mg, 98.7% p., 55% y.). m/z [M+H]⁺=661.3, ¹H NMR (600 MHz, DMSO-d₆): δ (ppm) 10.97 (s, 1H), 8.24-7.69 (m, 3H), 7.61 (br d, J=5.7 Hz, 1H), 7.51-7.21 (m, 7H), 7.20-7.05 (m, 1H), 7.01-6.86 (m, 1H), 5.21-4.96 (m, 2H), 4.59-4.46 (m, 1H), 4.45-4.13 (m, 5H), 3.31 (s, 3H), 3.04-2.69 (m, 3H), 2.66-2.53 (m, 2H), 2.43-2.18 (m, 4H), 2.05-1.92 (m, 1H), 1.68-0.73 (m, 4H), [α]_{D}=+5°·mL·dm⁻¹ g⁻¹[20° C., Na lamp, 589 nm, C=1.3 mg/mL, DMSO].

### Example 68

### (3S)-3-[5-[4-[(R)-[3-[(3R)-3-aminopyrrolidin-1-yl]-2-chloro-phenyl]-phenyl-methyl]piperidine-1-carbonyl]-6-methyl-1-oxo-isoindolin-2-yl]piperidine-2,6-dione

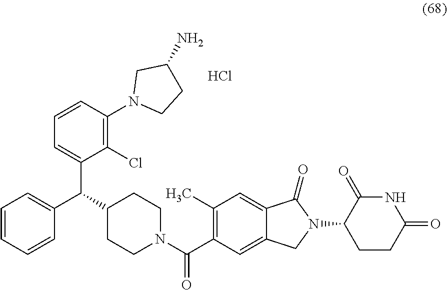 [SMILES: Cc1cc2c(cc1C(=O)N1CCC([C@H](c3ccccc3)c3cccc(N4CC[C@@H](N)C4)c3Cl)CC1)CN([C@H]1CCC(=O)NC1=O)C2=O ; Cl]

### Intermediate 68A: benzyl 4-[(R)-[3-[(3R)-3-(tert-butoxycarbonylamino)pyrrolidin-1-yl]-2-chloro-phenyl]-phenyl-methyl]piperidine-1-carboxylate

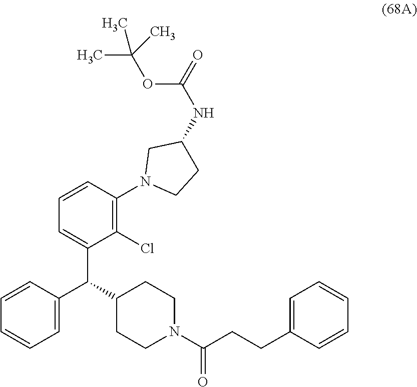 [SMILES: CC(C)(C)OC(=O)N[C@@H]1CCN(c2cccc([C@@H](c3ccccc3)C3CCN(C(=O)CCc4ccccc4)CC3)c2Cl)C1]

In a sealed vial, to a stirred solution of benzyl 4-[(R)-(3-bromo-2-chloro-phenyl)-phenyl-methyl]piperidine-1-carboxylate (250 mg, 0.476 mmol) in tert-butanol (2.4 mL) under argon were added successively t-BuONa (68.6 mg, 0.714 mmol) and tert-butyl N—[(3 {R})-pyrrolidin-3-yl]carbamate (181 mg, 0.952 mmol). Brettphos-Pd-G3 (22.7 mg, 0.0238 mmol) and dicyclohexyl-[3,6-dimethoxy-2-(2,4,6-triisopropylphenyl)phenyl]phosphane (13.0 mg, 0.0238 mmol) were added. The vial was sealed and heated at 80° C. for 2 h. The reaction was cooled and quenched by addition of water and EtOAc. The aqueous layer was extracted with EtOAc (2×). The combined organic layers were washed with brine, dried with Na₂SO₄, filtered, and concentrated under reduced pressure. The crude was purified by flash chromatography on silica gel using a gradient of EtOAc in cyclohexane from 0% to 50% to afford benzyl 4-[(R)-[3-[(3R)-3-(tert-butoxycarbonylamino)pyrrolidin-1-yl]-2-chloro-phenyl]-phenyl-methyl]piperidine-1-carboxylate as a brown oil (167 mg, 63% p., 37% y.). m/z [M+H]⁺=604.3, ¹H NMR (400 MHz, DMSO-d₆): δ (ppm) 7.40-7.09 (m, 13H), 6.84-6.76 (m, 1H), 5.05 (s, 2H), 4.21 (d, J=11.1 Hz, 1H), 3.96 (d, J=13.7 Hz, 3H), 3.41 (dd, J=9.6, 6.6 Hz, 1H), 3.27-3.21 (m, 2H), 3.10-3.00 (m, 1H), 2.77 (s, 3H), 2.43 (d, J=10.7 Hz, 1H), 2.06 (dd, J=12.6, 6.6 Hz, 1H), 1.77 (dd, J=12.4, 6.4 Hz, 1H), 1.48 (d, J=13.4 Hz, 1H), 1.38 (s, 9H), 1.02 (d, J=20.0 Hz, 2H).

### Intermediate 68B: tert-butyl N-[(3R)-1-[2-chloro-3-[(R)-phenyl(4-piperidyl)methyl]phenyl]pyrrolidin-3-yl]carbamate

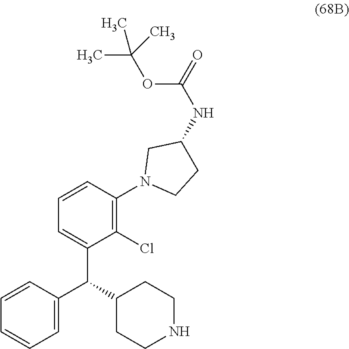 [SMILES: CC(C)(C)OC(=O)N[C@@H]1CCN(c2cccc([C@@H](c3ccccc3)C3CCNCC3)c2Cl)C1]

Palladium on carbon (10% w/w, 29.4 mg, 0.0276 mmol) was added to a solution of benzyl 4-[(R)-[3-[(3R)-3-(tert-butoxycarbonylamino)pyrrolidin-1-yl]-2-chloro-phenyl]-phenyl-methyl]piperidine-1-carboxylate (167 mg, 0.276 mmol) and AcOH (15.8 μL, 0.276 mmol) in EtOAc (7.9 mL). The reaction mixture was stirred under H₂(g) (0.3 bars) for 48 hours at ambient temperature. The reaction mixture was filtered through a pad of decalite and the filtrate was concentrated under reduced pressure to afford tert-butyl N-[(3R)-1-[2-chloro-3-[(R)-phenyl(4-piperidyl)methyl]phenyl]pyrrolidin-3-yl]carbamate as a beige solid (121 mg, 71% p., 66% y.). m/z [M+H]⁺=470.2.

### Intermediate 68C: tert-butyl(4S)-5-amino-4-[5-[4-[(R)-[3-[(3R)-3-(tert-butoxycarbonylamino)pyrrolidin-1-yl]-2-chloro-phenyl]-phenyl-methyl]piperidine-1-carbonyl]-6-methyl-1-oxo-isoindolin-2-yl]-5-oxo-pentanoate

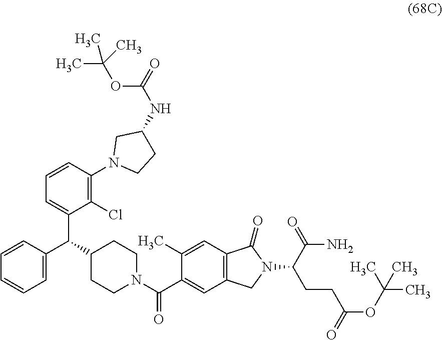 [SMILES: Cc1cc2c(cc1C(=O)N1CCC([C@H](c3ccccc3)c3cccc(N4CC[C@@H](NC(=O)OC(C)(C)C)C4)c3Cl)CC1)CN([C@@H](CCC(=O)OC(C)(C)C)C(N)=O)C2=O]

To a stirred solution of tert-butyl N-[(3R)-1-[2-chloro-3-[(R)-phenyl(4-piperidyl)methyl]phenyl]pyrrolidin-3-yl]carbamate (187 mg, 0.399 mmol) in DMF (4.0 mL) were added successively DIPEA (209 μL, 1.20 mmol), 2-[(1S)-4-tert-butoxy-1-carbamoyl-4-oxo-butyl]-6-methyl-1-oxo-isoindoline-5-carboxylic acid (150 mg, 0.399 mmol) and then HATU (227 mg, 0.598 mmol). The reaction mixture was stirred at ambient temperature overnight. The reaction was quenched with the addition of water. The aqueous layer was extracted with EtOAc (3×). The combined organic layers were washed with brine, dried over Na₂SO₄, filtered, and concentrated under reduced pressure. The crude was purified by flash chromatography on silica gel using a gradient of EtOAc/EtOH (3/1) in n-heptane from 1% to 100% to afford tert-butyl(4S)-5-amino-4-[5-[4-[(R)-[3-[(3R)-3-(tert-butoxycarbonylamino)pyrrolidin-1-yl]-2-chloro-phenyl]-phenyl-methyl]piperidine-1-carbonyl]-6-methyl-1-oxo-isoindolin-2-yl]-5-oxo-pentanoate as a pale yellow solid (25.8 mg, 82% p., 6% y.). m/z [M+H]⁺=828.4.

### Example 68

To a stirred solution of tert-butyl(4S)-5-amino-4-[5-[4-[(R)-[3-[(3R)-3-(tert-butoxycarbonylamino)pyrrolidin-1-yl]-2-chloro-phenyl]-phenyl-methyl]piperidine-1-carbonyl]-6-methyl-1-oxo-isoindolin-2-yl]-5-oxo-pentanoate (25.0 mg, 0.0302 mmol) in acetonitrile (300 μL) was added 4-methylbenzenesulfonic acid hydrate (57.4 mg, 0.302 mmol). The reaction mixture was stirred at 80° C. for 4 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). Acetonitrile was removed under reduced pressure. The aqueous layer was basified with a saturated aqueous solution of NaHCO₃ and extracted with EtOAc (3×). The combined organic layers were washed with brine, dried over Na₂SO₄, filtered, and concentrated under reduced pressure. The residue was solubilized in 1,4-dioxane (760 μL). Next, a 4 M solution of HCl in 1,4-dioxane (76 μL) was added dropwise. The reaction mixture was stirred for 30 min. Et₂O(8.3 mL) was added dropwise. The precipitate was triturated for 1 h, filtered, washed with Et₂O and dried under reduced pressure at 50° C. overnight to afford (3S)-3-[5-[4-[(R)-[3-[(3R)-3-aminopyrrolidin-1-yl]-2-chloro-phenyl]-phenyl-methyl]piperidine-1-carbonyl]-6-methyl-1-oxo-isoindolin-2-yl]piperidine-2,6-dione hydrochloride as an off-white solid (4.20 mg, 96% p., 19% y.). m/z [M+H]⁺=654.5, ¹H NMR (600 MHz, DMSO-d₆): δ (ppm) 11.01-10.93 (m, 1H), 8.15-7.98 (m, 3H), 7.67-7.57 (m, 1H), 7.51-7.09 (m, 8H), 6.96-6.81 (m, 1H), 5.15-5.04 (m, 1H), 4.57-4.46 (m, 1H), 4.45-4.36 (m, 1H), 4.34-4.20 (m, 2H), 3.87-3.74 (m, 1H), 3.51-3.36 (m, 2H), 3.31-3.12 (m, 3H), 3.06-2.83 (m, 2H), 2.83-2.68 (m, 1H), 2.65-2.52 (m, 2H), 2.46-2.16 (m, 5H), 2.05-1.82 (m, 2H), 1.70-0.87 (m, 4H).

### Example 69

### (3S)-3-[5-[4-[(R)-[3-[(1-aminocyclopropyl)methoxy]-2-chloro-phenyl]-phenyl-methyl]piperidine-1-carbonyl]-6-methyl-1-oxo-isoindolin-2-yl]piperidine-2,6-dione hydrochloride

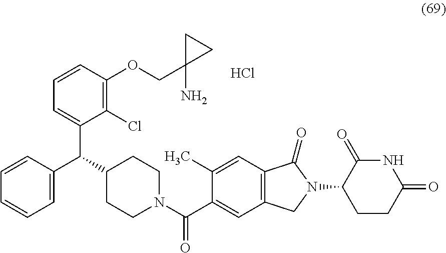 [SMILES: Cc1cc2c(cc1C(=O)N1CCC([C@H](c3ccccc3)c3cccc(OCC4(N)CC4)c3Cl)CC1)CN([C@H]1CCC(=O)NC1=O)C2=O ; Cl]

### Intermediate 69A: tert-butyl(4S)-5-amino-4-[5-[4-[(R)-[3-[1-(tert-butoxycarbonylamino) cyclopropyl]methoxy]-2-chloro-phenyl]-phenyl-methyl]piperidine-1-carbonyl]-6-methyl-1-oxo-isoindolin-2-yl]-5-oxo-pentanoate

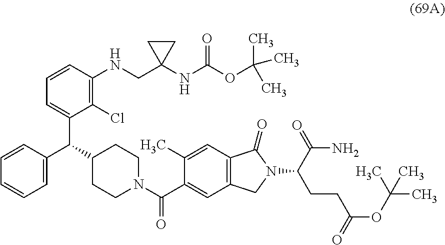 [SMILES: Cc1cc2c(cc1C(=O)N1CCC([C@H](c3ccccc3)c3cccc(NCC4(NC(=O)OC(C)(C)C)CC4)c3Cl)CC1)CN([C@@H](CCC(=O)OC(C)(C)C)C(N)=O)C2=O]

To a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.227 mmol) in dry THF (1.5 mL) were added successively triphenylphosphane (179 mg, 0.682 mmol), tert-butyl N-[1-(hydroxymethyl)cyclopropyl]carbamate (128 mg, 0.682 mmol), and then DIAD (134 μL, 0.682 mmol). The reaction mixture was stirred at ambient temperature for 16 h. Additional triphenylphosphane (119 mg, 0.454 mmol), tert-butyl N-[1-(hydroxymethyl)cyclopropyl]carbamate (85.1 mg, 0.454 mmol) and DIAD (89.5 L, 0.454 mmol) were added at ambient temperature. The reaction mixture was stirred at ambient temperature for 16 h. The reaction mixture was concentrated under reduced pressure. The crude was purified by flash chromatography on silica gel using a gradient of EtOAc/EtOH (3/1) (0.5% conc. NH₄OH) in n-heptane from 5% to 100% to afford tert-butyl(4S)-5-amino-4-[5-[4-[(R)-[3-[1-(tert-butoxycarbonylamino)cyclopropyl]methoxy]-2-chloro-phenyl]-phenyl-methyl]piperidine-1-carbonyl]-6-methyl-1-oxo-isoindolin-2-yl]-5-oxo-pentanoate as a white solid (124 mg, 86% p., 57% y.). m/z [M-Boc+2H]⁺=729.4.

### Example 69

To a stirred solution of tert-butyl(4S)-5-amino-4-[5-[4-[(R)-[3-[1-(tert-butoxycarbonylamino)cyclopropyl]methoxy]-2-chloro-phenyl]-phenyl-methyl]piperidine-1-carbonyl]-6-methyl-1-oxo-isoindolin-2-yl]-5-oxo-pentanoate (124 mg, 0.129 mmol) in acetonitrile (1.0 mL) at ambient temperature was added 4-methylbenzenesulfonic acid hydrate (73.4 mg, 0.386 mmol). The reaction mixture was stirred at 80° C. for 2 h. The reaction mixture was basified with a saturated aqueous solution of NaHCO₃ and EtOAc was added. The layers were separated, and the aqueous layer was extracted with EtOAc (2×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). Fractions were concentrated and basified with a saturated aqueous solution of NaHCO₃. The aqueous layer was extracted with EtOAc (2×). The combined organic layers were washed with brine, dried over Na₂SO₄, filtered, and concentrated under reduced pressure. The residue was solubilized in dry 1,4-dioxane (1.0 mL) at ambient temperature and a 4 M solution of HCl in 1,4-dioxane (321 μL, 1.29 mmol) was added dropwise. The mixture was stirred at ambient temperature for 10 min and then Et₂O was added. The resulting precipitate was triturated for 30 min, filtered, washed with Et₂O, and dried under reduced pressure at 50° C. for 12 h to afford (3S)-3-[5-[4-[(R)-[3-[(1-aminocyclopropyl)methoxy]-2-chloro-phenyl]-phenyl-methyl]piperidine-1-carbonyl]-6-methyl-1-oxo-isoindolin-2-yl]piperidine-2,6-dione hydrochloride as a white powder (43.2 mg, 100% p., 49% y.). m/z [M+H]⁺=655.4, ¹H NMR (600 MHz, DMSO-d₆): δ (ppm) 10.97 (s, 1H), 8.37 (br d, J=1.0 Hz, 3H), 7.65-7.57 (m, 1H), 7.49-7.21 (m, 7H), 7.20-7.08 (m, 1H), 7.01-6.89 (m, 1H), 5.22-4.96 (m, 1H), 4.59-4.47 (m, 1H), 4.46-4.36 (m, 1H), 4.35-4.25 (m, 1H), 4.25-4.18 (m, 1H), 4.17-3.99 (m, 2H), 3.27-3.15 (m, 1H), 3.04-2.94 (m, 1H), 2.94-2.83 (m, 1H), 2.83-2.70 (m, 1H), 2.63-2.53 (m, 2H), 2.43-2.21 (m, 4H), 2.04-1.93 (m, 1H), 1.67-1.23 (m, 2H), 1.21-0.87 (m, 6H), [α]_{D}=+4°·mL·dm⁻¹ g^{-1 [20}° C., Na lamp, 589 nm, C=1.5 mg/mL, DMSO].

### Example 70

### (3S)-3-[5-[4-[(R)-[2-chloro-3-[(3R)-3-hydroxypyrrolidin-1-yl]phenyl]-phenyl-methyl]piperidine-1-carbonyl]-6-methyl-1-oxo-isoindolin-2-yl]piperidine-2,6-dione

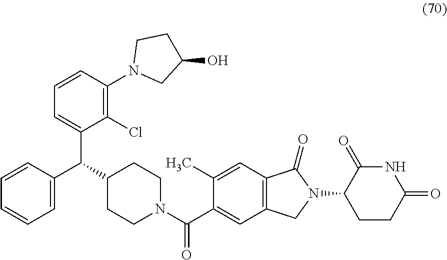 [SMILES: Cc1cc2c(cc1C(=O)N1CCC([C@H](c3ccccc3)c3cccc(N4CC[C@@H](O)C4)c3Cl)CC1)CN([C@H]1CCC(=O)NC1=O)C2=O]

### Intermediate 70A: benzyl 4-[(R)-[3-[(3R)-3-[tert-butyl(dimethyl)silyl]oxypyrrolidin-1-yl]-2-chloro-phenyl]-phenyl-methyl]piperidine-1-carboxylate

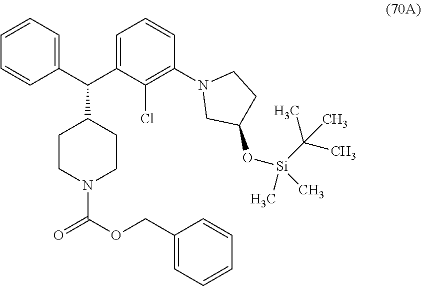 [SMILES: CC(C)(C)[Si](C)(C)O[C@@H]1CCN(c2cccc([C@@H](c3ccccc3)C3CCN(C(=O)OCc4ccccc4)CC3)c2Cl)C1]

In a sealed vial, to a stirred solution of benzyl 4-[(R)-(3-bromo-2-chloro-phenyl)-phenyl-methyl]piperidine-1-carboxylate (200 mg, 0.381 mmol) in dry 1,4-dioxane (2.7 mL) were added under argon, (3R)-3-[(tert-butyldimethylsilyl)oxy]pyrrolidine (161 mg, 0.762 mmol), Cs₂CO₃ (380 mg, 1.14 mmol), and [1-(2-diphenylphosphanyl-1-naphthyl)-2-naphthyl]-diphenyl-phosphane (24.5 mg, 0.038 mmol). The reaction vessel was purged with argon for 10 min after which Pd(OAc)₂(10.4 mg, 0.046 mmol) was added. The reaction mixture was stirred at 100° C. overnight and at ambient temperature for 5 days. The suspension was filtered over glass fiber and rinsed with EtOAc. Water was added to the filtrate, the layers were separated, and the aqueous layer was extracted with EtOAc (2×). The combined organic layers were washed with brine, dried using a phase separator cartridge, and concentrated under reduced pressure. The crude was purified by flash chromatography on silica gel using a gradient of EtOAc in n-heptane from 5% to 40%. The desired fractions were combined and concentrated under reduced pressure to afford benzyl 4-[(R)-[3-[(3R)-3-[tert-butyl(dimethyl)silyl]oxypyrrolidin-1-yl]-2-chloro-phenyl]-phenyl-methyl]piperidine-1-carboxylate as an off-white foam (190 mg, 90% p., 73% y.). m/z [M+H]⁺=619.5, ¹H NMR (400 MHz, DMSO-d₆): δ (ppm) 7.38-7.24 (m, 9H), 7.20-7.13 (m, 3H), 6.88-6.79 (m, 1H), 5.05 (s, 2H), 4.46 (tt, J=5.5, 3.9 Hz, 1H), 4.22 (d, J=11.2 Hz, 1H), 3.96 (d, J=12.7 Hz, 2H), 3.53 (dd, J=10.0, 5.5 Hz, 1H), 3.30 (s, 1H), 3.21 (ddd, J=9.1, 7.4, 5.1 Hz, 1H), 2.98 (dd, J=10.0, 3.6 Hz, 1H), 2.72 (d, J=40.8 Hz, 2H), 2.43 (d, J=11.2 Hz, 1H), 2.11-1.98 (m, 1H), 1.75 (dq, J=11.8, 5.4 Hz, 1H), 1.48 (d, J=13.1 Hz, 1H), 1.38 (d, J=13.2 Hz, 1H), 1.09-0.91 (m, 2H), 0.84 (s, 9H), 0.06 (s, 3H), 0.04 (s, 3H), [α]_{D}=+55°·mL·dm⁻¹·g⁻¹[20° C., Na lamp, 589 nm, C=1.7 mg/mL, EtOH].

### Intermediate 70B: (3R)-1-[2-chloro-3-[(R)-phenyl(4-piperidyl)methyl]phenyl]pyrrolidin-3-ol dihydroiodide

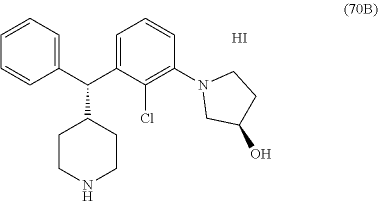

In a round-bottom flask, to a stirred solution of benzyl 4-[(R)-[3-[(3R)-3-[tert-butyl(dimethyl)silyl]oxypyrrolidin-1-yl]-2-chloro-phenyl]-phenyl-methyl]piperidine-1-carboxylate (185 mg, 0.299 mmol) in acetonitrile (1.4 mL) at ambient temperature under nitrogen was added iodo (trimethyl) silane (128 μL, 0.896 mmol) dropwise. The reaction mixture was stirred at ambient temperature for 30 min. MeOH (145 μL, 3.58 mmol) was added. The mixture was stirred at ambient temperature for 2 h and was concentrated under reduced pressure. Et₂O was added and the resulting mixture was stirred at ambient temperature for 2 h. The solid was filtered, washed with Et₂O, and dried under reduced pressure at 45° C. overnight to afford (3R)-1-[2-chloro-3-[(R)-phenyl(4-piperidyl)methyl]phenyl]pyrrolidin-3-ol dihydroiodide as a yellow solid (215 mg, 80% p., 92% y.). m/z [M+H]⁺=371.3, ¹H NMR (400 MHz, DMSO-d₆): δ (ppm) 8.40 (s, 1H), 8.08 (d, J=11.6 Hz, 1H), 7.38-7.34 (m, 2H), 7.32-7.27 (m, 2H), 7.23-7.14 (m, 2H), 7.12 (dd, J=7.8, 1.5 Hz, 1H), 6.84 (dd, J=8.2, 1.5 Hz, 1H), 4.30 (td, J=5.3, 2.6 Hz, 1H), 4.23 (d, J=11.3 Hz, 2H), 3.58 (dd, J=10.2, 5.2 Hz, 1H), 3.48-3.33 (m, 2H), 3.23 (d, J=12.6 Hz, 2H), 3.18-3.09 (m, 1H), 2.95 (dd, J=10.2, 2.9 Hz, 1H), 2.84 (s, 2H), 2.55 (d, J=11.2 Hz, 1H), 2.02-1.89 (m, 1H), 1.77 (ddt, J=11.5, 7.3, 4.0 Hz, 1H), 1.57 (dd, J=28.5, 14.0 Hz, 2H), 1.34-1.18 (m, 2H).

### Intermediate 70C: tert-butyl(4S)-5-amino-4-[5-[4-[(R)-[2-chloro-3-[(3R)-3-hydroxypyrrolidin-1-yl]phenyl]-phenyl-methyl]piperidine-1-carbonyl]-6-methyl-1-oxo-isoindolin-2-yl]-5-oxo-pentanoate

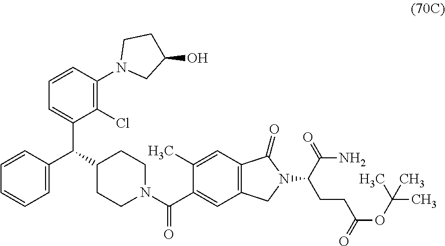 [SMILES: Cc1cc2c(cc1C(=O)N1CCC([C@H](c3ccccc3)c3cccc(N4CC[C@@H](O)C4)c3Cl)CC1)CN([C@@H](CCC(=O)OC(C)(C)C)C(N)=O)C2=O]

In a round-bottom flask, to a stirred solution of (3R)-1-[2-chloro-3-[(R)-phenyl(4-piperidyl)methyl]phenyl]pyrrolidin-3-ol dihydroiodide (215 mg, 0.274 mmol) in DMF (2.1 mL) were added 2-[(1S)-4-tert-butoxy-1-carbamoyl-4-oxo-butyl]-6-methyl-1-oxo-isoindoline-5-carboxylic acid (108 mg, 0.288 mmol), DIPEA (240 μL, 1.37 mmol), and HATU (323 mg, 0.823 mmol). The mixture was stirred at ambient temperature for 1 h. The reaction was quenched with the addition of water. EtOAc and then FastWorX® powder were added. The organic solvent was evaporated under reduced pressure. The reaction mixture was filtered through a dry-loading cartridge and the resin was rinsed with water. The loading cartridge was flushed with nitrogen and then loaded on flash chromatography on silica gel using a gradient of EtOAc/EtOH (3/1) in n-heptane from 5% to 100% to afford tert-butyl(4S)-5-amino-4-[5-[4-[(R)-[2-chloro-3-[(3R)-3-hydroxypyrrolidin-1-yl]phenyl]-phenyl-methyl]piperidine-1-carbonyl]-6-methyl-1-oxo-isoindolin-2-yl]-5-oxo-pentanoate as a yellow solid (172 mg, 77% p., 66% y.). m/z [M+H]⁺=729.4.

### Example 70

To a stirred solution of tert-butyl(4S)-5-amino-4-[5-[4-[(R)-[2-chloro-3-[(3R)-3-hydroxypyrrolidin-1-yl]phenyl]-phenyl-methyl]piperidine-1-carbonyl]-6-methyl-1-oxo-isoindolin-2-yl]-5-oxo-pentanoate (172 mg, 0.181 mmol) in acetonitrile (1.0 mL) at ambient temperature was added 4-methylbenzenesulfonic acid hydrate (104 mg, 0.544 mmol). The reaction mixture was stirred at 80° C. for 2 h. The reaction mixture was basified with a saturated aqueous solution of NaHCO₃ and EtOAc was added. The layers were separated and the aqueous layer was extracted with EtOAc (2×). The combined organic layers were washed with brine, dried over Na₂SO₄, filtered, and concentrated under reduced pressure. The crude was purified by flash chromatography on silica gel using a gradient of EtOAc/EtOH (3/1) (0.1% NH₃) in n-heptane from 10% to 100%. The residue was solubilized in a minimum of acetone and added dropwise to n-pentane. The resulting precipitate was filtered, washed with n-pentane, and dried under reduced pressure at 50° C. for 12 h to afford (3S)-3-[5-[4-[(R)-[2-chloro-3-[(3R)-3-hydroxypyrrolidin-1-yl]phenyl]-phenyl-methyl]piperidine-1-carbonyl]-6-methyl-1-oxo-isoindolin-2-yl]piperidine-2,6-dione as a pale yellow powder (58.0 mg, 92.4% p., 45% y.). m/z [M+H]⁺=655.4, ¹H NMR (600 MHz, DMSO-d₆): δ (ppm) 10.97 (s, 1H), 7.62-7.58 (m, 1H), 7.49-7.42 (m, 1H), 7.41-7.32 (m, 2H), 7.31-7.21 (m, 2H), 7.20-7.10 (m, 3H), 6.86-6.76 (m, 1H), 5.13-5.05 (m, 1H), 4.50 (br s, 1H), 4.41 (td, J=16.6, 8.2 Hz, 1H), 4.34-4.22 (m, 3H), 3.60-3.49 (m, 2H), 3.36-3.35 (m, 1H), 3.26-3.20 (m, 1H), 3.19-3.08 (m, 2H), 3.01-2.85 (m, 3H), 2.79-2.71 (m, 1H), 2.61-2.51 (m, 1H), 2.42-2.30 (m, 2H), 2.24 (s, 2H), 2.02-1.90 (m, 2H), 1.79-1.70 (m, 1H), 1.68-1.21 (m, 2H), 1.15-0.92 (m, 2H), [α]_{D}=+39°·mL·dm⁻¹ g⁻¹[20° C., Na lamp, 589 nm, C=1.4 mg/mL, DMSO].

### Example 71

### (3S)-3-[5-[4-[(R)-[3-[(2R)-3-amino-2-fluoro-propoxy]-2-chloro-phenyl]-phenyl-methyl]piperidine-1-carbonyl]-6-methyl-1-oxo-isoindolin-2-yl]piperidine-2,6-dione hydrochloride

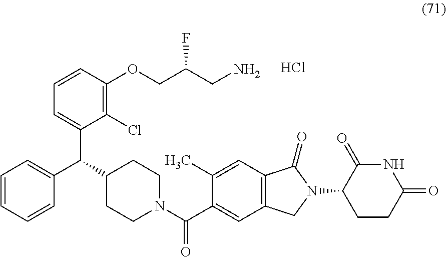 [SMILES: Cc1cc2c(cc1C(=O)N1CCC([C@H](c3ccccc3)c3cccc(OC[C@H](F)CN)c3Cl)CC1)CN([C@H]1CCC(=O)NC1=O)C2=O ; Cl]

### Intermediate 71A: tert-butyl(4S)-5-amino-4-[5-[4-[(R)-[3-[(2R)-3-(tert-butoxycarbonylamino)-2-fluoro-propoxy]-2-chloro-phenyl]-phenyl-methyl]piperidine-1-carbonyl]-6-methyl-1-oxo-isoindolin-2-yl]-5-oxo-pentanoate

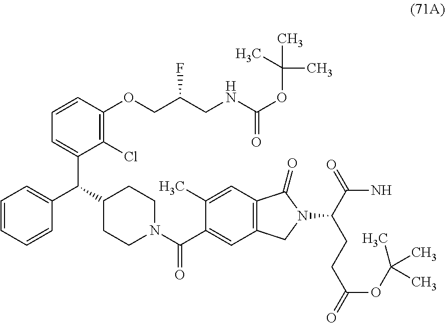 [SMILES: Cc1cc2c(cc1C(=O)N1CCC([C@H](c3ccccc3)c3cccc(OC[C@H](F)CNC(=O)OC(C)(C)C)c3Cl)CC1)CN([C@@H](CCC(=O)OC(C)(C)C)C(N)=O)C2=O]

To a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.227 mmol) in dry THF (1.6 mL) were added successively triphenylphosphane (179 mg, 0.682 mmol), tert-butyl N-[(2R)-2-fluoro-3-hydroxypropyl]carbamate (139 mg, 0.682 mmol), and then DIAD (134 μL, 0.682 mmol). The reaction mixture was stirred at ambient temperature overnight. The reaction mixture was concentrated under reduced pressure. The crude was purified by flash chromatography on silica gel using a gradient of EtOAc/EtOH (3/1) in n-heptane from 5% to 100% to afford tert-butyl(4S)-5-amino-4-[5-[4-[(R)-[3-[(2R)-3-(tert-butoxycarbonylamino)-2-fluoro-propoxy]-2-chloro-phenyl]-phenyl-methyl]piperidine-1-carbonyl]-6-methyl-1-oxo-isoindolin-2-yl]-5-oxo-pentanoate as a white solid (139 mg, 93% p., 68% y.). m/z [M-Boc+2H]⁺=735.3. [α]_{D}=+14° mL·dm⁻¹ g⁻¹[20° C., Na lamp, 589 nm, C=1.4 mg/mL, EtOH].

### Example 71

To a stirred solution of tert-butyl(4S)-5-amino-4-[5-[4-[(R)-[3-[(2R)-3-(tert-butoxycarbonylamino)-2-fluoro-propoxy]-2-chloro-phenyl]-phenyl-methyl]piperidine-1-carbonyl]-6-methyl-1-oxo-isoindolin-2-yl]-5-oxo-pentanoate (139 mg, 0.155 mmol) in acetonitrile (1.0 mL) at ambient temperature was added 4-methylbenzenesulfonic acid hydrate (88.3 mg, 0.464 mmol). The reaction mixture was stirred at 80° C. for 2 h. The reaction mixture was basified with a saturated aqueous solution of NaHCO₃ and EtOAc was added. The layers were separated, and the aqueous layer was extracted with EtOAc (2×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). Fractions were concentrated and basified with a saturated aqueous solution of NaHCO₃. The aqueous layer was extracted with EtOAc (2×). The combined organic layers were washed with brine, dried over Na₂SO₄, filtered, and concentrated under reduced pressure. The residue was solubilized in dry 1,4-dioxane (1.0 mL) at ambient temperature and a 4 M solution of HCl in 1,4-dioxane (387 μL, 1.55 mmol) was added dropwise. The mixture was stirred at ambient temperature for 10 min and then Et₂O was added. The resulting precipitate was triturated for 30 min, filtered, washed with Et₂O, and dried under reduced pressure at 50° C. for 12 h to afford (3S)-3-[5-[4-[(R)-[3-[(2R)-3-amino-2-fluoro-propoxy]-2-chloro-phenyl]-phenyl-methyl]piperidine-1-carbonyl]-6-methyl-1-oxo-isoindolin-2-yl]piperidine-2,6-dione hydrochloride as a white powder (70.1 mg, 99.5% p., 65% y.). m/z [M+H]⁺=661.5, ¹H NMR (600 MHz, DMSO-d₆): δ (ppm) 10.97 (s, 1H), 8.15 (br s, 3H), 7.65-7.57 (m, 1H), 7.48-7.20 (m, 7H), 7.20-7.09 (m, 1H), 7.00-6.89 (m, 1H), 5.23-5.02 (m, 2H), 4.57-4.46 (m, 1H), 4.45-4.13 (m, 5H), 3.30-3.15 (m, 3H), 3.04-2.84 (m, 2H), 2.84-2.68 (m, 1H), 2.64-2.53 (m, 2H), 2.45-2.20 (m, 4H), 1.98 (s, 1H), 1.67-1.23 (m, 2H), 1.21-0.83 (m, 2H), [α]_{D}=−6° mL·dm⁻¹ g⁻¹[20° C., Na lamp, 589 nm, C=0.9 mg/mL, DMSO].

### Example 72

### (3S)-3-[5-[4-[(R)-[2-chloro-3-[(3R)-1-(2-fluoroethyl)pyrrolidin-3-yl]oxy-phenyl]-phenyl-methyl]piperidine-1-carbonyl]-6-methyl-1-oxo-isoindolin-2-yl]piperidine-2,6-dione hydrochloride

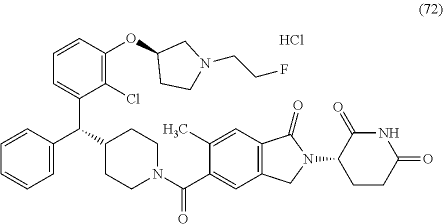 [SMILES: Cc1cc2c(cc1C(=O)N1CCC([C@H](c3ccccc3)c3cccc(O[C@@H]4CCN(CCF)C4)c3Cl)CC1)CN([C@H]1CCC(=O)NC1=O)C2=O ; Cl]

### Intermediate 72A: (3S)-1-(2-fluoroethyl)pyrrolidin-3-ol

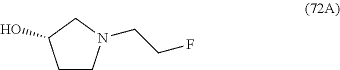

To a stirred solution of (3S)-pyrrolidin-3-ol hydrochloride (1:1) (500 mg, 4.05 mmol) in dry acetonitrile (8.0 mL) at ambient temperature were added K₂CO₃ (1.68 g, 12.1 mmol) and then 2-fluoroethyl 4-methylbenzenesulfonate (739 μL, 4.25 mmol). The reaction mixture was stirred at 80° C. for 16 h. The reaction mixture was filtered and concentrated. The crude was solubilized in MeOH and NaHCO₃ supported was added until pH=8. The mixture was filtered, washed with MeOH, and concentrated under reduced pressure to afford (3S)-1-(2-fluoroethyl)pyrrolidin-3-ol as a brown oil (584 mg, 95% p., quant. yield)). m/z [M+H]⁺=134.1, ¹H NMR (400 MHz, DMSO-d₆): δ (ppm) 5.34 (s, 1H), 4.74 (dt, J=47.4, 4.7 Hz, 2H), 4.35 (hept, J=2.8 Hz, 1H), 3.37 (t, J=2.5 Hz, 1H), 3.32-3.08 (m, 4H), 2.97 (dd, J=11.5, 2.7 Hz, 1H), 2.14-2.01 (m, 1H), 1.85-1.73 (m, 1H).

### Intermediate 72B: tert-butyl(4S)-5-amino-4-[5-[4-[(R)-[2-chloro-3-[(3R)-1-(2-fluoroethyl)pyrrolidin-3-yl]oxy-phenyl]-phenyl-methyl]piperidine-1-carbonyl]-6-methyl-1-oxo-isoindolin-2-yl]-5-oxo-pentanoate

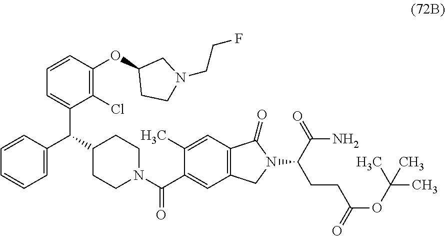 [SMILES: Cc1cc2c(cc1C(=O)N1CCC([C@H](c3ccccc3)c3cccc(O[C@@H]4CCN(CCF)C4)c3Cl)CC1)CN([C@@H](CCC(=O)OC(C)(C)C)C(N)=O)C2=O]

To a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.227 mmol) in dry THF (1.5 mL) were added successively triphenylphosphane (179 mg, 0.682 mmol), (3S)-1-(2-fluoroethyl)pyrrolidin-3-ol (95.5 mg, 0.682 mmol), and then DIAD (134 μL, 0.682 mmol). The reaction mixture was stirred at ambient temperature for 3 days. Additional (3S)-1-(2-fluoroethyl)pyrrolidin-3-ol (47.8 mg, 0.341 mmol), triphenylphosphane (89.4 mg, 0.341 mmol), and DIAD (67 μL, 0.341 mmol) were added at ambient temperature and the reaction mixture was stirred at ambient temperature for 16 h. The reaction mixture was concentrated under reduced pressure. The crude was purified by flash chromatography on silica gel using a gradient of EtOAc/EtOH (3/1) (0.5% conc. NH₄OH) in n-heptane from 5% to 100% to afford tert-butyl(4S)-5-amino-4-[5-[4-[(R)-[2-chloro-3-[(3R)-1-(2-fluoroethyl)pyrrolidin-3-yl]oxy-phenyl]-phenyl-methyl]piperidine-1-carbonyl]-6-methyl-1-oxo-isoindolin-2-yl]-5-oxo-pentanoate as a brown solid (57 mg, 90% p., 29% y.). m/z [M+H]⁺=775.4.

### Example 72

To a stirred solution of tert-butyl(4S)-5-amino-4-[5-[4-[(R)-[2-chloro-3-[(3R)-1-(2-fluoroethyl)pyrrolidin-3-yl]oxy-phenyl]-phenyl-methyl]piperidine-1-carbonyl]-6-methyl-1-oxo-isoindolin-2-yl]-5-oxo-pentanoate (57.0 mg, 0.067 mmol) in acetonitrile (1.0 mL) at ambient temperature was added 4-methylbenzenesulfonic acid hydrate (38.2 mg, 0.201 mmol). The reaction mixture was stirred at 80° C. for 2 h. Additional 4-methylbenzenesulfonic acid hydrate (38.2 mg, 0.201 mmol) was added at ambient temperature and the reaction mixture was stirred at 80° C. for 1 h. The reaction mixture was basified with a saturated aqueous solution of NaHCO₃ and EtOAc was added. The layers were separated, and the aqueous layer was extracted with EtOAc (2×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The fractions were concentrated and basified with a saturated aqueous solution of NaHCO₃. The aqueous layer was extracted with EtOAc (2×). The combined organic layers were washed with brine, dried over Na₂SO₄, filtered, and concentrated under reduced pressure. The residue was solubilized in dry 1,4-dioxane (1.0 mL) at ambient temperature and a 4 M solution of HCl in 1,4-dioxane (167 μL) was added dropwise. The mixture was stirred at ambient temperature for 10 min and then Et₂O was added. The resulting precipitate was triturated for 30 min, filtered, washed with Et₂O, and dried under reduced pressure at 50° C. for 12 h to afford (3S)-3-[5-[4-[(R)-[2-chloro-3-[(3R)-1-(2-fluoroethyl)pyrrolidin-3-yl]oxy-phenyl]-phenyl-methyl]piperidine-1-carbonyl]-6-methyl-1-oxo-isoindolin-2-yl]piperidine-2,6-dione hydrochloride as an off-white powder (8.4 mg, 98.7% p., 17% y.). m/z [M+H]⁺=701.4, ¹H NMR (600 MHz, DMSO-d₆): δ (ppm) 10.97 (s, 1H), 10.93-10.37 (m, 1H), 7.62 (s, 1H), 7.49-7.39 (m, 1H), 7.34 (s, 2H), 7.34-7.33 (m, 1H), 7.32-7.21 (m, 3H), 7.21-7.10 (m, 1H), 7.04-6.89 (m, 1H), 5.22-5.01 (m, 2H), 4.94-4.64 (m, 2H), 4.52 (br dd, J=5.2, 3.3 Hz, 1H), 4.46-4.36 (m, 1H), 4.33 (br d, J=1.3 Hz, 1H), 4.20 (br d, J=11.9 Hz, 1H), 4.08-3.75 (m, 1H), 3.75-3.59 (m, 2H), 3.56-3.42 (m, 1H), 3.41-3.34 (m, 1H), 3.28-3.23 (m, 1H), 3.23-3.15 (m, 1H), 3.00 (br s, 1H), 2.94-2.85 (m, 1H), 2.84-2.71 (m, 1H), 2.70-2.54 (m, 2H), 2.43-2.35 (m, 1H), 2.35-2.23 (m, 4H), 2.22-2.05 (m, 1H), 2.04-1.93 (m, 1H), 1.66-1.25 (m, 2H), 1.22-0.85 (m, 2H).

### Example 73

### (3S)-3-[5-[4-[(R)-[2-chloro-3-(1H-imidazol-5-ylmethoxy)phenyl]-phenyl-methyl]piperidine-1-carbonyl]-6-methyl-1-oxo-isoindolin-2-yl]piperidine-2,6-dione hydrochloride

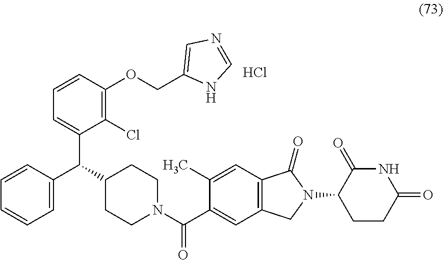 [SMILES: Cc1cc2c(cc1C(=O)N1CCC([C@H](c3ccccc3)c3cccc(OCc4cnc[nH]4)c3Cl)CC1)CN([C@H]1CCC(=O)NC1=O)C2=O ; Cl]

### Intermediate 73A: tert-butyl(4S)-5-amino-4-[5-[4-[(R)-[2-chloro-3-[[3-(2-trimethylsilylethoxymethyl) imidazol-4-yl]methoxy]phenyl]-phenyl-methyl]piperidine-1-carbonyl]-6-methyl-1-oxo-isoindolin-2-yl]-5-oxo-pentanoate

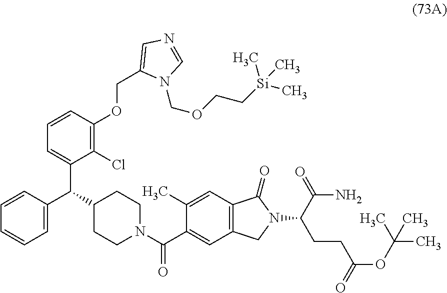 [SMILES: Cc1cc2c(cc1C(=O)N1CCC([C@H](c3ccccc3)c3cccc(OCc4cncn4COCC[Si](C)(C)C)c3Cl)CC1)CN([C@@H](CCC(=O)OC(C)(C)C)C(N)=O)C2=O]

To a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (200 mg, 0.303 mmol) and [3-(2-trimethylsilylethoxymethyl) imidazol-4-yl]methanol (415 mg, 1.82 mmol) in THF (3.0 mL) were added successively triphenylphosphane (477 mg, 1.82 mmol) and then DBAD (419 mg, 1.82 mmol). The reaction mixture was stirred at ambient temperature overnight. The reaction mixture was concentrated under reduced pressure. The crude was purified by flash chromatography on silica gel using a gradient of EtOAc/EtOH (3/1) in n-heptane from 10% to 100% to afford (tert-butyl(4S)-5-amino-4-[5-[4-[(R)-[2-chloro-3-[3-(2-trimethylsilylethoxymethyl) imidazol-4-yl]methoxy]phenyl]-phenyl-methyl]piperidine-1-carbonyl]-6-methyl-1-oxo-isoindolin-2-yl]-5-oxo-pentanoate as a yellow solid (103 mg, 80% p., 31% y.). m/z [M+H]⁺=870.4.

### Intermediate 73B: (4S)-5-amino-4-[5-[4-[(R)-[2-chloro-3-(1H-imidazol-5-ylmethoxy)phenyl]-phenyl-methyl]piperidine-1-carbonyl]-6-methyl-1-oxo-isoindolin-2-yl]-5-oxo-pentanoic acid

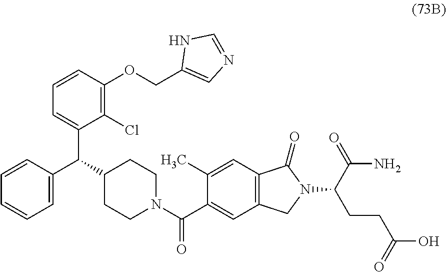 [SMILES: Cc1cc2c(cc1C(=O)N1CCC([C@H](c3ccccc3)c3cccc(OCc4cnc[nH]4)c3Cl)CC1)CN([C@@H](CCC(=O)O)C(N)=O)C2=O]

To a stirred solution of tert-butyl(4S)-5-amino-4-[5-[4-[(R)-[2-chloro-3-[3-(2-trimethylsilylethoxymethyl) imidazol-4-yl]methoxy]phenyl]-phenyl-methyl]piperidine-1-carbonyl]-6-methyl-1-oxo-isoindolin-2-yl]-5-oxo-pentanoate (100 mg, 0.115 mmol) in THF (2.3 mL) was added TBAF (230 mg, 0.345 mmol). The reaction mixture was stirred at reflux for 1.5 h. The reaction mixture was concentrated under reduced pressure and dried under reduced pressure. To a stirred solution of the crude in DCM (350 μL) was added TFA (352 μL, 4.59 mmol). The reaction mixture was stirred at ambient temperature overnight. The crude was purified by reverse-phase preparative chromatography using a gradient of acetonitrile in water from 10% to 100% (0.1% AcOH). The desired fractions were combined and concentrated under reduced pressure to afford (4S)-5-amino-4-[5-[4-[(R)-[2-chloro-3-(1H-imidazol-5-ylmethoxy)phenyl]-phenyl-methyl]piperidine-1-carbonyl]-6-methyl-1-oxo-isoindolin-2-yl]-5-oxo-pentanoic acid as an orange gum (37.6 mg, 84% p., 40% y.). m/z [M+H]⁺=684.3.

### Example 73

To a stirred solution of (4S)-5-amino-4-[5-[4-[(R)-[2-chloro-3-(1H-imidazol-5-ylmethoxy)phenyl]-phenyl-methyl]piperidine-1-carbonyl]-6-methyl-1-oxo-isoindolin-2-yl]-5-oxo-pentanoic acid (37.0 mg, 0.054 mmol) in acetonitrile (540 μL) was added 4-methylbenzenesulfonic acid hydrate (103 mg, 0.540 mmol). The reaction mixture was stirred at 80° C. for 4 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). Acetonitrile was removed under reduced pressure. The aqueous layer was basified with a saturated aqueous solution of NaHCO₃. The aqueous layer was extracted with EtOAc (3×). The combined organic layers were washed with brine, dried over Na₂SO₄, filtered, and concentrated under reduced pressure. The residue was solubilized in 1,4-dioxane (1.4 mL). a 4 M solution of HCl in 1,4-dioxane (140 μL) was added dropwise. The reaction mixture was stirred for 30 min. Et₂O(15 mL) was added dropwise. The precipitate was triturated for 1 h, filtered, washed with Et₂O and dried under reduced pressure at 50° C. overnight to afford (3S)-3-[5-[4-[(R)-[2-chloro-3-(1H-imidazol-5-ylmethoxy)phenyl]-phenyl-methyl]piperidine-1-carbonyl]-6-methyl-1-oxo-isoindolin-2-yl]piperidine-2,6-dione hydrochloride as an off-white solid (1.0 mg, 89% p., 2% y.). m/z [M+H]⁺=666.3, ¹H NMR (600 MHz, DMSO-d₆): δ (ppm) 14.58-13.97 (m, 1H), 10.97 (s, 1H), 9.05-8.64 (m, 1H), 7.77-7.67 (m, 1H), 7.62-7.59 (m, 1H), 7.47-7.41 (m, 1H), 7.40-7.26 (m, 5H), 7.26-7.21 (m, 1H), 7.19-7.09 (m, 2H), 5.21-5.05 (m, 3H), 4.51 (br s, 1H), 4.44-4.37 (m, 1H), 4.33-4.26 (m, 1H), 4.25-4.12 (m, 1H), 3.23 (br d, J=13.1 Hz, 2H), 2.99 (br d, J=12.6 Hz, 1H), 2.89 (br d, J=5.7 Hz, 1H), 2.61-2.51 (m, 2H), 2.41-2.30 (m, 3H), 2.24 (br d, J=2.8 Hz, 2H), 2.05-1.93 (m, 1H), 1.69-0.82 (m, 4H).

### Example 74

### (3S)-3-[5-[4-[(R)-[2-chloro-3-(oxazol-5-ylmethoxy)phenyl]-phenyl-methyl]piperidine-1-carbonyl]-6-methyl-1-oxo-isoindolin-2-yl]piperidine-2,6-dione

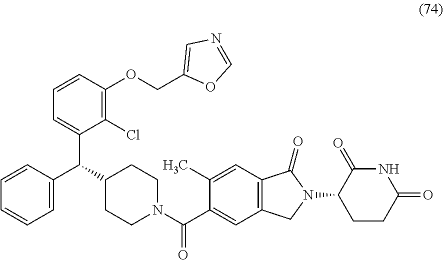 [SMILES: Cc1cc2c(cc1C(=O)N1CCC([C@H](c3ccccc3)c3cccc(OCc4cnco4)c3Cl)CC1)CN([C@H]1CCC(=O)NC1=O)C2=O]

### Intermediate 74A: tert-butyl(4S)-5-amino-4-[5-[4-[(R)-[2-chloro-3-(oxazol-5-ylmethoxy)phenyl]-phenyl-methyl]piperidine-1-carbonyl]-6-methyl-1-oxo-isoindolin-2-yl]-5-oxo-pentanoate

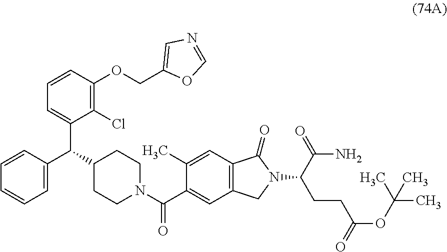 [SMILES: Cc1cc2c(cc1C(=O)N1CCC([C@H](c3ccccc3)c3cccc(OCc4cnco4)c3Cl)CC1)CN([C@@H](CCC(=O)OC(C)(C)C)C(N)=O)C2=O]

To a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.211 mmol) and 1,3-oxazol-5-ylmethanol (65.4 mg, 0.634 mmol) in dry THF (1.4 mL) were added successively triphenylphosphane (166 mg, 0.634 mmol) and then DBAD (150 mg, 0.634 mmol). The reaction mixture was stirred at ambient temperature for 84 h. The reaction mixture was concentrated under reduced pressure. The crude was purified by flash chromatography on silica gel using a gradient of EtOAc/EtOH (3/1) (0.5% conc. NH₄OH) in n-heptane from 5% to 100% to afford tert-butyl(4S)-5-amino-4-[5-[4-[(R)-[2-chloro-3-(oxazol-5-ylmethoxy)phenyl]-phenyl-methyl]piperidine-1-carbonyl]-6-methyl-1-oxo-isoindolin-2-yl]-5-oxo-pentanoate as a white solid (117 mg, 87% p., 65% y.). m/z [M+H]⁺=741.3.

### Example 74

In a sealed vial, to a solution of tert-butyl(4S)-5-amino-4-[5-[4-[(R)-[2-chloro-3-(oxazol-5-ylmethoxy)phenyl]-phenyl-methyl]piperidine-1-carbonyl]-6-methyl-1-oxo-isoindolin-2-yl]-5-oxo-pentanoate (113 mg, 0.133 mmol) in acetonitrile (2.3 mL) was added 4-methylbenzenesulfonic acid hydrate (252 mg, 1.33 mmol). The solution was stirred at 90° C. for 3 h. The mixture was diluted with EtOAc and washed with a saturated aqueous solution of NaHCO₃ and brine. The organic layer was dried over Na₂SO₄, filtered, and concentrated under reduced pressure. The crude was purified by reverse-phase preparative chromatography using a gradient of acetonitrile in water from 0% to 100% (0.1% AcOH). The desired fractions were combined and concentrated under reduced pressure until most of the acetonitrile evaporated. A saturated aqueous solution of NaHCO₃ was added. The resulting suspension was filtered and dried under at 50° C. under reduced pressure for 48 hours to afford (3S)-3-[5-[4-[(R)-[2-chloro-3-(oxazol-5-ylmethoxy)phenyl]-phenyl-methyl]piperidine-1-carbonyl]-6-methyl-1-oxo-isoindolin-2-yl]piperidine-2,6-dione as an off-white powder (12.6 mg, 91.3% p., 13% y.). m/z [M+H]⁺=667.1, ¹H NMR (500 MHz, DMSO-d₆): δ (ppm) 10.98 (s, 1H), 8.46-8.33 (m, 1H), 7.65-7.57 (m, 1H), 7.48-7.07 (m, 10H), 5.34-5.03 (m, 3H), 4.59-4.10 (m, 4H), 3.27-3.17 (m, 1H), 3.07-2.52 (m, 5H), 2.45-2.20 (m, 4H), 2.08-1.89 (m, 1H), 1.70-1.12 (m, 4H), [α]_{D}=+52° mL·dm⁻¹·g⁻¹[20° C., Na lamp, 589 nm, C=1.1 mg/mL, DMSO].

### Example 75

### (3S)-3-[5-[4-[(R)-[2-chloro-3-(oxazol-4-ylmethoxy)phenyl]-phenyl-methyl]piperidine-1-carbonyl]-6-methyl-1-oxo-isoindolin-2-yl]piperidine-2,6-dione

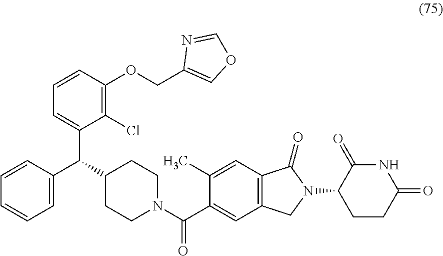 [SMILES: Cc1cc2c(cc1C(=O)N1CCC([C@H](c3ccccc3)c3cccc(OCc4cocn4)c3Cl)CC1)CN([C@H]1CCC(=O)NC1=O)C2=O]

### Intermediate 75A: tert-butyl(4S)-5-amino-4-[5-[4-[(R)-[2-chloro-3-(oxazol-4-ylmethoxy)phenyl]-phenyl-methyl]piperidine-1-carbonyl]-6-methyl-1-oxo-isoindolin-2-yl]-5-oxo-pentanoate

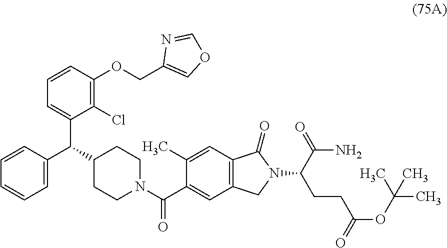 [SMILES: Cc1cc2c(cc1C(=O)N1CCC([C@H](c3ccccc3)c3cccc(OCc4cocn4)c3Cl)CC1)CN([C@@H](CCC(=O)OC(C)(C)C)C(N)=O)C2=O]

To a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.211 mmol) and 1,3-oxazol-4-ylmethanol (65.4 mg, 0.634 mmol) in dry THF (1.4 mL) were added successively triphenylphosphane (166 mg, 0.634 mmol), and then DBAD (150 mg, 0.634 mmol). The reaction mixture was stirred at ambient temperature for 84 h. The reaction mixture was concentrated under reduced pressure. The crude was purified by flash chromatography on silica gel using a gradient of EtOAc/EtOH (3/1) (0.5% conc. NH₄OH) in n-heptane from 5% to 100% to afford tert-butyl(4S)-5-amino-4-[5-[4-[(R)-[2-chloro-3-(oxazol-4-ylmethoxy)phenyl]-phenyl-methyl]piperidine-1-carbonyl]-6-methyl-1-oxo-isoindolin-2-yl]-5-oxo-pentanoate as a white solid (110 mg, 96% p., 67% y.). m/z [M+H]⁺=741.3. [α]_{D}=+27° mL. dm⁻¹ g⁻¹[20° C., Na lamp, 589 nm, C=1.7 mg/mL, EtOH].

### Example 75

To a stirred solution of tert-butyl(4S)-5-amino-4-[5-[4-[(R)-[2-chloro-3-(oxazol-4-ylmethoxy)phenyl]-phenyl-methyl]piperidine-1-carbonyl]-6-methyl-1-oxo-isoindolin-2-yl]-5-oxo-pentanoate (110 mg, 0.142 mmol) in acetonitrile (1.0 mL) at ambient temperature was added 4-methylbenzenesulfonic acid hydrate (81.3 mg, 0.427 mmol). The reaction mixture was stirred at 80° C. for 3 h. The reaction mixture was basified with a saturated aqueous solution of NaHCO₃ and EtOAc was added. The layers were separated, and the aqueous layer extracted with EtOAc (2×). The combined organic layers were washed with brine, dried over Na₂SO₄, filtered, and concentrated under reduced pressure. The crude was purified by flash chromatography on silica gel using a gradient of EtOAc/EtOH (3/1) (0.1% NH₃) in n-heptane from 10% to 100%. The residue was solubilized in a minimum of acetone and added dropwise to n-pentane. The resulting precipitate was filtered, washed with n-pentane, and dried under reduced pressure at 50° C. for 12 h to afford (3S)-3-[5-[4-[(R)-[2-chloro-3-(oxazol-4-ylmethoxy)phenyl]-phenyl-methyl]piperidine-1-carbonyl]-6-methyl-1-oxo-isoindolin-2-yl]piperidine-2,6-dione as a white powder (33.0 mg, 94.8% p., 33% y.). m/z [M+H]⁺=667.2, ¹H NMR (600 MHz, DMSO-d₆): δ (ppm) 10.97 (s, 1H), 8.42-8.36 (m, 1H), 8.23-8.17 (m, 1H), 7.64-7.57 (m, 1H), 7.50-7.41 (m, 1H), 7.37 (br d, J=7.2 Hz, 1H), 7.35-7.21 (m, 5H), 7.19-7.08 (m, 2H), 5.12-4.99 (m, 3H), 4.50 (br s, 1H), 4.46-4.35 (m, 1H), 4.33-4.17 (m, 2H), 3.26-3.17 (m, 1H), 3.01-2.85 (m, 2H), 2.79-2.71 (m, 1H), 2.61-2.55 (m, 2H), 2.41-2.30 (m, 2H), 2.24 (s, 2H), 2.02-1.95 (m, 1H), 1.66-0.90 (m, 4H), [α]_{D}=+10° mL·dm⁻¹ g⁻¹[20° C., Na lamp, 589 nm, C=2.1 mg/mL, DMSO].

### Example 76

### (3S)-3-[5-[4-[rel-(S)-indolin-4-yl(phenyl)methyl]piperidine-1-carbonyl]-6-methyl-1-oxo-isoindolin-2-yl]piperidine-2,6-dione

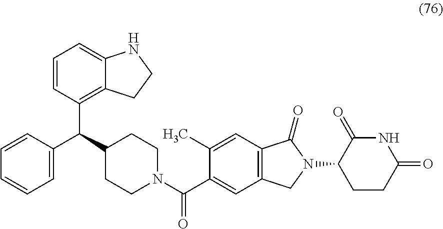 [SMILES: Cc1cc2c(cc1C(=O)N1CCC([C@@H](c3ccccc3)c3cccc4c3CCN4)CC1)CN([C@H]1CCC(=O)NC1=O)C2=O]

### Intermediate 76A: tert-butyl 4-bromoindoline-1-carboxylate

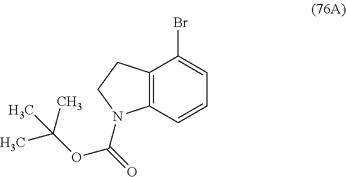 [SMILES: CC(C)(C)OC(=O)N1CCc2c(Br)cccc21]

In a round-bottom flask, to a stirred solution of 4-bromo-2,3-dihydro-1H-indole (650 mg, 3.12 mmol) in THF (6.2 mL) was added tert-butoxycarbonyl tert-butyl carbonate (842 mg, 3.74 mmol) followed by DMAP (38.1 mg, 0.312 mmol). The resulting mixture was stirred at ambient temperature overnight. The reaction was quenched with the addition of water. EtOAc was added to the mixture. The layers were separated, and the aqueous layer was extracted with EtOAc (2×). The combined organic layers were washed with brine, dried using a phase separator cartridge, and concentrated under reduced pressure. The crude was purified by flash chromatography on silica gel using a gradient of EtOAc in n-heptane from 0% to 30%. The desired fractions were combined and concentrated under reduced pressure to afford tert-butyl 4-bromoindoline-1-carboxylate as a white solid (806 mg, 99% p., 86% y.). m/z [M-tBu+2H]⁺=242.1, 1H NMR (400 MHz, DMSO-d₆): δ (ppm) 7.62 (s, 1H), 7.18-7.06 (m, 2H), 3.94 (dd, J=9.3, 8.1 Hz, 2H), 3.03 (t, J=8.7 Hz, 2H), 1.50 (s, 9H).

### Intermediate 76B: tert-butyl 4-(4,4,5,5-tetramethyl-1,3,2-dioxaborolan-2-yl) indoline-1-carboxylate

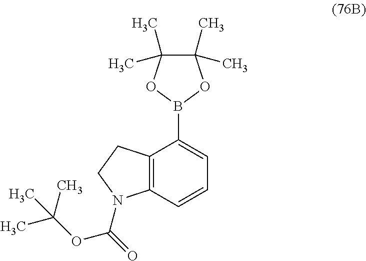 [SMILES: CC(C)(C)OC(=O)N1CCc2c(B3OC(C)(C)C(C)(C)O3)cccc21]

In a sealed vial, to a stirred solution of tert-butyl 4-bromoindoline-1-carboxylate (500 mg, 1.68 mmol) in dry 1,4-dioxane (9.0 mL) under argon were added successively B₂pin₂(652 mg, 2.52 mmol) and KOAc (494 mg, 5.03 mmol). The suspension was degassed with argon for 5 min and Pd(dppf)Cl₂(123 mg, 0.168 mmol) was added. The mixture was stirred at 90° C. overnight. The suspension was filtered through a glass fiber filter and rinsed with EtOAc. A saturated aqueous solution of NaHCO₃ was added and the layer was extracted with EtOAc (3×). The combined organic layers were washed with brine, dried using a phase separator cartridge, and concentrated under reduced pressure. The crude was purified by flash chromatography on silica gel using a gradient of EtOAc in n-heptane from 0% to 30%. The desired fractions were combined and concentrated under reduced pressure to afford tert-butyl 4-(4,4,5,5-tetramethyl-1,3,2-dioxaborolan-2-yl) indoline-1-carboxylate as a white gum (478 mg, 98% p., 81% y.). m/z [M-tBu+2H]=290.2, ¹H NMR (400 MHz, DMSO-d₆): δ (ppm) 7.78 (s, 1H), 7.21 (dd, J=7.5, 1.2 Hz, 1H), 7.14 (t, J=7.7 Hz, 1H), 3.88 (dd, J=9.2, 8.2 Hz, 2H), 3.17 (t, J=8.7 Hz, 2H), 1.50 (s, 9H), 1.29 (s, 12H).

### Intermediate 76C: tert-butyl 4-[(1-benzyloxycarbonyl-4-piperidylidene)-phenyl-methyl]indoline-1-carboxylate

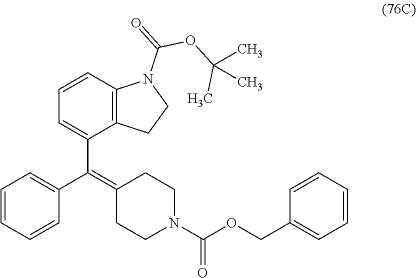 [SMILES: CC(C)(C)OC(=O)N1CCc2c(C(=C3CCN(C(=O)OCc4ccccc4)CC3)c3ccccc3)cccc21]

In a sealed vial, to a stirred solution of benzyl 4-[bromo (phenyl)methylene]piperidine-1-carboxylate (450 mg, 1.16 mmol) in 1,4-dioxane (8.1 mL) and water (2.7 mL) were added under argon tert-butyl 4-(4,4,5,5-tetramethyl-1,3,2-dioxaborolan-2-yl) indoline-1-carboxylate (483 mg, 1.40 mmol) and K₃PO₄ (502 mg, 2.33 mmol). The reaction mixture was degassed with argon for 5 min after which Pd(PPh3) 4 (137 mg, 0.116 mmol) was added. The reaction mixture was stirred at 90° C. for 4 h. EtOAc was added and the layers were separated. The aqueous layer was extracted with EtOAc (2×). The combined organic layers were washed with brine, dried using a phase separator cartridge, and concentrated under reduced pressure. The crude was purified by flash chromatography on silica gel using a gradient of EtOAc in n-heptane from 0% to 40%. The desired fractions were combined and concentrated under reduced pressure to afford tert-butyl 4-[(1-benzyloxycarbonyl-4-piperidylidene)-phenyl-methyl]indoline-1-carboxylate as a white foam (559 mg, 98% p., 90% y.). m/z [M-Boc+2H]⁺=425.4, 1H NMR (400 MHz, DMSO-d₆): δ (ppm) 7.56 (s, 1H), 7.38-7.28 (m, 7H), 7.24-7.19 (m, 1H), 7.17-7.10 (m, 3H), 6.76 (dd, J=7.7, 1.0 Hz, 1H), 5.09 (s, 2H), 3.82 (s, 2H), 3.45 (s, 4H), 2.82 (s, 1H), 2.59 (s, 1H), 2.31 (s, 2H), 2.11 (d, J=6.2 Hz, 2H), 1.48 (s, 9H).

### Intermediate 76D: tert-butyl 4-[phenyl(4-piperidyl)methyl]indoline-1-carboxylate

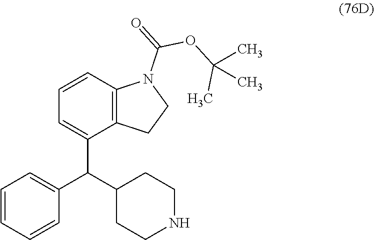 [SMILES: CC(C)(C)OC(=O)N1CCc2c(C(c3ccccc3)C3CCNCC3)cccc21]

In a sealed vial, to a stirred solution of tert-butyl 4-[(1-benzyloxycarbonyl-4-piperidylidene)-phenyl-methyl]indoline-1-carboxylate (300 mg, 0.572 mmol) in EtOAc (4.1 mL) under argon was added AcOH (32.7 μL, 0.572 mmol). The suspension was degassed with argon for 5 min and palladium on carbon (10% w/w, 304 mg, 0.0286 mmol) and triethylsilane (554 μL, 3.43 mmol) were added. The mixture was stirred at 70° C. for 5 h. The suspension was filtered through a glass fiber filter and rinsed with EtOAc. The filtrate was basified with a saturated aqueous solution of NaHCO₃ and EtOAc was added. The layers were separated, and the aqueous layer extracted with EtOAc (2×). The combined organic layers were washed with brine, dried using a phase separator cartridge, and concentrated under reduced pressure to afford tert-butyl 4-[phenyl(4-piperidyl)methyl]indoline-1-carboxylate as a pale brown oil (495 mg, 50% p., quant. yield)). m/z [M+H]⁺=393.4, ¹H NMR (400 MHz, DMSO-d₆): δ (ppm) 7.47 (s, 1H), 7.34-7.21 (m, 4H), 7.19-7.06 (m, 3H), 5.17 (s, 3H), 3.87 (pd, J=10.9, 6.8 Hz, 2H), 3.52 (d, J=11.0 Hz, 1H), 3.21-3.12 (m, 1H), 2.93 (dd, J=18.8, 9.5 Hz, 3H), 2.50-2.42 (m, 4H), 2.29 (q, J=11.2 Hz, 1H), 1.47 (s, 9H).

### Intermediate 76E: tert-butyl 4-[rel-(S)-[1-[2-[(1S)-4-tert-butoxy-1-carbamoyl-4-oxo-butyl]-6-methyl-1-oxo-isoindoline-5-carbonyl]-4-piperidyl]-phenyl-methyl]indoline-1-carboxylate

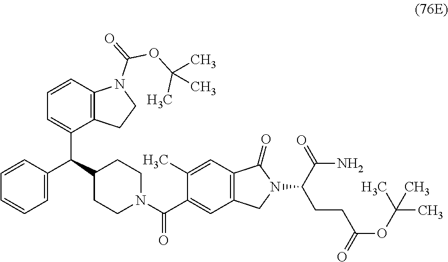 [SMILES: Cc1cc2c(cc1C(=O)N1CCC([C@@H](c3ccccc3)c3cccc4c3CCN4C(=O)OC(C)(C)C)CC1)CN([C@@H](CCC(=O)OC(C)(C)C)C(N)=O)C2=O]

In a round-bottom flask, to a solution of tert-butyl 4-[phenyl(4-piperidyl)methyl]indoline-1-carboxylate (490 mg, 0.624 mmol) in DMF (4.0 mL) were added 2-[(1S)-4-tert-butoxy-1-carbamoyl-4-oxo-butyl]-6-methyl-1-oxo-isoindoline-5-carboxylic acid (235 mg, 0.624 mmol), DIPEA (327 μL, 1.87 mmol), and HATU (726 mg, 1.87 mmol). The mixture was stirred at ambient temperature for 4 h. The reaction was quenched with the addition of water. The mixture was basified with saturated aqueous solution of NaHCO₃. EtOAc and then FastWorX® powder were added. The organic solvent was evaporated under reduced pressure. The reaction mixture was filtered through a dry-loading cartridge and the resin was rinsed with water. The loading cartridge was flushed with nitrogen. The crude was purified by flash chromatography on silica gel using a gradient of EtOAc/EtOH (3/1) in n-heptane from 5% to 100% to afford tert-butyl 4-[1-[2-[(1S)-4-tert-butoxy-1-carbamoyl-4-oxo-butyl]-6-methyl-1-oxo-isoindoline-5-carbonyl]-4-piperidyl]-phenyl-methyl]indoline-1-carboxylate as a pale yellow foam (439 mg, 99% p., 93% y.). The product was purified by chiral supercritical chromatography (Waters Prep S200, Chiralpak AD (5 μm, 250×4.6 mm), CO₂/iPrOH 60/40 as mobile phase, flow rate 2.4 mL/min, 104 bars). The resulting colorless oil was solubilized, co-evaporated with EtOAc (2×), and concentrated under reduced pressure to afford (second isomer eluted) as an off-white solid (tert-butyl 4-[rel-(S)-[1-[2-[(1S)-4-tert-butoxy-1-carbamoyl-4-oxo-butyl]-6-methyl-1-oxo-isoindoline-5-carbonyl]-4-piperidyl]-phenyl-methyl]indoline-1-carboxylate (118 mg, 100% p., 25% y., d.e.=99.4%)) m/z [M+Na]⁺=773.4. [α]_{D}=−28° mL·dm⁻¹·g⁻¹[20° C., Na lamp, 589 nm, C=1.4 mg/mL, EtOH].

### Example 76

In a sealed vial, to a solution of tert-butyl 4-[rel-(S)-[1-[2-[(1S)-4-tert-butoxy-1-carbamoyl-4-oxo-butyl]-6-methyl-1-oxo-isoindoline-5-carbonyl]-4-piperidyl]-phenyl-methyl]indoline-1-carboxylate (118 mg, 0.157 mmol) in acetonitrile (2.7 mL) was added 4-methylbenzenesulfonic acid hydrate (179 mg, 0.943 mmol). The solution was stirred at 90° C. for 2 h. The mixture was diluted with EtOAc and washed with a saturated aqueous solution of NaHCO₃ and brine. The organic layer was dried over Na₂SO₄, filtered, and concentrated under reduced pressure. The crude was purified by reverse-phase preparative chromatography using a gradient of acetonitrile in water from 0% to 100% (0.1% AcOH). The desired fractions were combined and concentrated under reduced pressure. A saturated aqueous solution of NaHCO₃ was added. The resulting suspension was filtered and dried under reduced pressure at 50° C. for 48 hours to afford (3S)-3-[5-[4-[rel-(S)-indolin-4-yl(phenyl)methyl]piperidine-1-carbonyl]-6-methyl-1-oxo-isoindolin-2-yl]piperidine-2,6-dione as a white powder (35.4 mg, 98.6% p., 39% y.). m/z [M+H]⁺=577.2, ¹H NMR (600 MHz, DMSO-d₆): δ (ppm) 11.03-10.86 (m, 1H), 7.64-7.56 (m, 1H), 7.45-7.19 (m, 5H), 7.18-7.07 (m, 1H), 6.94-6.78 (m, 1H), 6.76-6.63 (m, 1H), 6.31-6.20 (m, 1H), 5.44-5.31 (m, 1H), 5.09 (br dd, J=13.3, 4.9 Hz, 1H), 4.56-4.46 (m, 1H), 4.46-4.20 (m, 2H), 3.58-3.52 (m, 1H), 3.44-3.32 (m, 2H), 3.27-2.54 (m, 7H), 2.49-2.20 (m, 5H), 2.04-1.92 (m, 1H), 1.81-0.82 (m, 4H), [α]_{D}=−34° mL·dm⁻¹ g⁻¹[20° C., Na lamp, 589 nm, C=0.5 mg/mL, DMSO].

### Example 77

### (3S)-3-[5-[4-[rel-(R)-indolin-4-yl(phenyl)methyl]piperidine-1-carbonyl]-6-methyl-1-oxo-isoindolin-2-yl]piperidine-2,6-dione

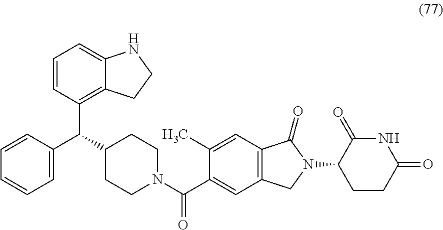 [SMILES: Cc1cc2c(cc1C(=O)N1CCC([C@H](c3ccccc3)c3cccc4c3CCN4)CC1)CN([C@H]1CCC(=O)NC1=O)C2=O]

### Intermediate 77A: tert-butyl 4-[rel-(R)-[1-[2-[(1S)-4-tert-butoxy-1-carbamoyl-4-oxo-butyl]-6-methyl-1-oxo-isoindoline-5-carbonyl]-4-piperidyl]-phenyl-methyl]indoline-1-carboxylate

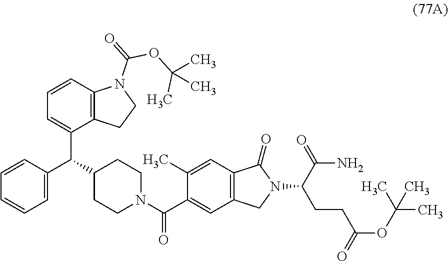 [SMILES: Cc1cc2c(cc1C(=O)N1CCC([C@H](c3ccccc3)c3cccc4c3CCN4C(=O)OC(C)(C)C)CC1)CN([C@@H](CCC(=O)OC(C)(C)C)C(N)=O)C2=O]

In a round-bottom flask, to a solution of tert-butyl 4-[phenyl(4-piperidyl)methyl]indoline-1-carboxylate (490 mg, 0.624 mmol) in DMF (4.0 mL) was added 2-[(1S)-4-tert-butoxy-1-carbamoyl-4-oxo-butyl]-6-methyl-1-oxo-isoindoline-5-carboxylic acid (235 mg, 0.624 mmol), DIPEA (327 μL, 1.87 mmol), and HATU (726 mg, 1.87 mmol). The mixture was stirred at ambient temperature for 4 h. The reaction was quenched with the addition of water. The mixture was basified with saturated aqueous solution of NaHCO₃. EtOAc and then FastWorX® powder were added. The organic solvent was evaporated under reduced pressure. The reaction mixture was filtered through a dry-loading cartridge and the resin was rinsed with water. The loading cartridge was flushed with nitrogen. The crude was purified by flash chromatography on silica gel using a gradient of EtOAc/EtOH (3/1) in n-heptane from 5% to 100% to afford tert-butyl 4-[1-[2-[(1S)-4-tert-butoxy-1-carbamoyl-4-oxo-butyl]-6-methyl-1-oxo-isoindoline-5-carbonyl]-4-piperidyl]-phenyl-methyl]indoline-1-carboxylate as a pale yellow foam (439 mg, 99% p., 93% y.). The product was purified by chiral supercritical chromatography (Waters Prep SFC200, Chiralpak AD (5 μm, 250×4.6 mm), CO₂/iPrOH 60/40 as mobile phase, flow rate 2.4 mL/min, 104 bars). The resulting colorless oil was solubilized, co-eluted with EtOAc (2×) and concentrated under reduced pressure to afford (first isomer eluted) as a white solid tert-butyl 4-[rel-(R)-[1-[2-[(1S)-4-tert-butoxy-1-carbamoyl-4-oxo-butyl]-6-methyl-1-oxo-isoindoline-5-carbonyl]-4-piperidyl]-phenyl-methyl]indoline-1-carboxylate (120 mg, 100% p., 26% y., d.e.>99.9%)). m/z [M+Na]⁺=773.5. [α]_{D}=+9° mL·dm⁻¹ g^{-1 [20}° C., Na lamp, 589 nm, C=1.5 mg/mL, EtOH].

### Example 77

In a sealed vial, to a solution of tert-butyl 4-[rel-(R)-[1-[2-[(1S)-4-tert-butoxy-1-carbamoyl-4-oxo-butyl]-6-methyl-1-oxo-isoindoline-5-carbonyl]-4-piperidyl]-phenyl-methyl]indoline-1-carboxylate (120 mg, 0.160 mmol) in acetonitrile (2.7 mL) was added 4-methylbenzenesulfonic acid hydrate (182 mg, 0.959 mmol). The solution was stirred at 90° C. for 2 h. The mixture was diluted with EtOAc and washed with a saturated aqueous solution of NaHCO₃ and brine. The organic layer was dried over Na₂SO₄, filtered, and concentrated under reduced pressure. The crude was purified by reverse-phase preparative chromatography using a gradient of acetonitrile in water from 0% to 100% (0.1% AcOH). The desired fractions were combined and concentrated under reduced pressure. A saturated aqueous solution of NaHCO₃ was added. The resulting suspension was filtered and dried under at 50° C. under reduced pressure for 48 hours to afford (3S)-3-[5-[4-[rel-(R)-indolin-4-yl(phenyl)methyl]piperidine-1-carbonyl]-6-methyl-1-oxo-isoindolin-2-yl]piperidine-2,6-dione as a white powder (37.7 mg, 97.2% p., 40% y.). m/z [M+H]⁺=577.3, ¹H NMR (600 MHz, DMSO-d₆): δ (ppm) 11.06-10.84 (m, 1H), 7.62-7.56 (m, 1H), 7.44-7.18 (m, 5H), 7.18-7.07 (m, 1H), 6.92-6.81 (m, 1H), 6.77-6.57 (m, 1H), 6.32-6.14 (m, 1H), 5.48-5.28 (m, 1H), 5.15-4.99 (m, 1H), 4.51 (br s, 1H), 4.46-4.22 (m, 2H), 3.60-3.50 (m, 1H), 3.43-3.32 (m, 2H), 3.26-3.15 (m, 1H), 3.10-2.54 (m, 6H), 2.48-2.42 (m, 1H), 2.41-2.21 (m, 4H), 2.05-1.87 (m, 1H), 1.81-0.83 (m, 4H), [α]_{D}=+40° mL·dm⁻¹·g⁻¹[20° C., Na lamp, 589 nm, C=0.5 mg/mL, DMSO].

### Example 78

### (3S)-3-[5-[4-[(R)-[3-[(2S)-2-amino-3,3,3-trifluoro-propoxy]-2-chloro-phenyl]-phenyl-methyl]piperidine-1-carbonyl]-6-methyl-1-oxo-isoindolin-2-yl]piperidine-2,6-dione

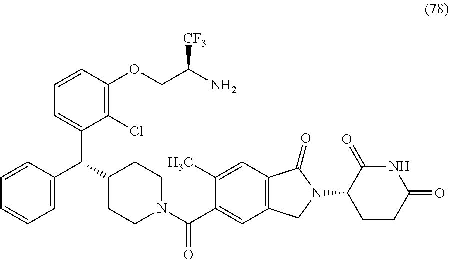 [SMILES: Cc1cc2c(cc1C(=O)N1CCC([C@H](c3ccccc3)c3cccc(OC[C@H](N)C(F)(F)F)c3Cl)CC1)CN([C@H]1CCC(=O)NC1=O)C2=O]

### Intermediate 78A: benzyl 4-[(R)-[3-[(2S)-2-(tert-butoxycarbonylamino)-3,3,3-trifluoro-propoxy]-2-chloro-phenyl]-phenyl-methyl]piperidine-1-carboxylate

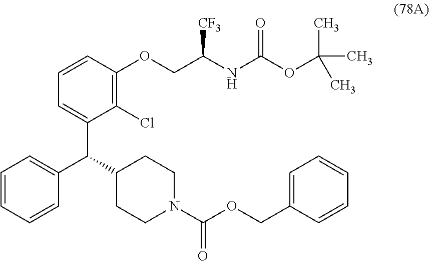 [SMILES: CC(C)(C)OC(=O)N[C@@H](COc1cccc([C@@H](c2ccccc2)C2CCN(C(=O)OCc3ccccc3)CC2)c1Cl)C(F)(F)F]

A vial was successively charged with benzyl 4-[(R)-(3-bromo-2-chloro-phenyl)-phenyl-methyl]piperidine-1-carboxylate (250 mg, 0.486 mmol), tert-butyl N-[(2S)-1,1,1-trifluoro-3-hydroxypropan-2-yl]carbamate (292 mg, 1.25 mmol), Ir[dF(CF₃)ppy]₂(dtbbpy)PF₆ (5.5 mg, 0.0486 mmol), and 4,4′-di-tert-butyl-2,2′-bipyridine dichloronickel (10.2 mg, 0.024 mmol) in EtOAc (5.1 mL). The reaction mixture was degassed with argon for 10 min. Next, 2,2,6,6-tetramethylpiperidine (82 μL, 0.486 mmol) was added and the reaction mixture was irradiated with a blue LEDs (456 nm) without ventilation overnight. Additional tert-butyl N-[(2S)-1,1,1-trifluoro-3-hydroxypropan-2-yl]carbamate (295 mg, 1.25 mmol), Ir[dF(CF₃)ppy]₂(dtbbpy)PF₆ (5.5 mg, 0.0486 mmol), and 4,4′-di-tert-butyl-2,2′-bipyridine dichloronickel (10.2 mg, 0.024 mmol) were added. The reaction mixture was degassed with argon for 5 min. Next, 2,2,6,6-tetramethylpiperidine (82 μL, 0.486 mmol) was added and the reaction mixture was irradiated with a blue LEDs (456 nm) without ventilation overnight. The reaction was quenched with the addition of a saturated aqueous solution of NH₄Cl. EtOAc was added to the mixture. The layers were separated and the aqueous layer extracted with EtOAc (2×). The combined organic layers were washed with brine, dried using hydrophobic paper, and concentrated under reduced pressure. The crude was purified by flash chromatography on silica gel using a gradient of EtOAc in n-heptane from 0% to 100% to afford benzyl 4-[(R)-[3-[(2S)-2-(tert-butoxycarbonylamino)-3,3,3-trifluoro-propoxy]-2-chloro-phenyl]-phenyl-methyl]piperidine-1-carboxylate as a yellow oil (627 mg, 62% p., quant. yield)). m/z [M+Na]⁺=669.3.

### Intermediate 78B: tert-butyl N-[(1S)-1-[2-chloro-3-[(R)-phenyl(4-piperidyl)methyl]phenoxy]methyl]-2,2,2-trifluoro-ethyl]carbamate

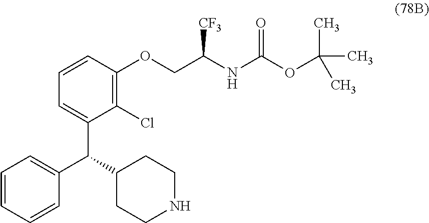 [SMILES: CC(C)(C)OC(=O)N[C@@H](COc1cccc([C@@H](c2ccccc2)C2CCNCC2)c1Cl)C(F)(F)F]

To a solution of benzyl 4-[(R)-[3-[(2S)-2-(tert-butoxycarbonylamino)-3,3,3-trifluoro-propoxy]-2-chloro-phenyl]-phenyl-methyl]piperidine-1-carboxylate (627 mg, 0.601 mmol) in EtOH (9.2 mL), was added palladium on carbon (10% w/w, 95.9 mg, 0.0901 mmol). The reaction mixture was stirred under H₂(g) (0.3 bars) overnight at ambient temperature. The suspension was filtered, washed with EtOH, and dried under reduced pressure to afford tert-butyl N-[(1S)-1-[2-chloro-3-[(R)-phenyl(4-piperidyl)methyl]phenoxy]methyl]-2,2,2-trifluoro-ethyl]carbamate as a yellow oil (548 mg, 76% p., quant. yield)). m/z [M+H]⁺=513.3.

### Intermediate 78C: tert-butyl(4S)-5-amino-4-[5-[4-[(R)-[3-[(2S)-2-(tert-butoxycarbonylamino)-3,3,3-trifluoro-propoxy]-2-chloro-phenyl]-phenyl-methyl]piperidine-1-carbonyl]-6-methyl-1-oxo-isoindolin-2-yl]-5-oxo-pentanoate

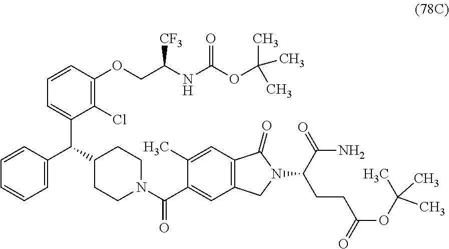 [SMILES: Cc1cc2c(cc1C(=O)N1CCC([C@H](c3ccccc3)c3cccc(OC[C@H](NC(=O)OC(C)(C)C)C(F)(F)F)c3Cl)CC1)CN([C@@H](CCC(=O)OC(C)(C)C)C(N)=O)C2=O]

To a stirred suspension of 2-[(1S)-4-tert-butoxy-1-carbamoyl-4-oxo-butyl]-6-methyl-1-oxo-isoindoline-5-carboxylic acid (338 mg, 0.852 mmol) and tert-butyl N-[(1S)-1-[2-chloro-3-[(R)-phenyl(4-piperidyl)methyl]phenoxy]methyl]-2,2,2-trifluoro-ethyl]carbamate (548 mg, 0.812 mmol) in DMF (7.2 mL) were added successively DIPEA (567 μL, 3.25 mmol) and HATU (477 mg, 1.22 mmol). The reaction mixture was stirred at ambient temperature for 2 h. The reaction was quenched with the addition of a saturated aqueous solution of NH₄Cl. The aqueous layer was extracted with EtOAc (2×). The combined organic layers were washed with a saturated aqueous solution of NaHCO₃ and brine, dried over hydrophobic paper, and concentrated under reduced pressure. The crude was purified by flash chromatography on silica gel using a gradient of EtOAc/EtOH (3/1) in n-heptane from 2% to 100% and by reverse-phase preparative chromatography using a gradient of acetonitrile in water from 0% to 100% (0.1% AcOH). Acetonitrile was removed under reduced pressure. The solution was basified with a saturated aqueous solution of NaHCO₃ and EtOAc was added. The layers were separated, and the aqueous layer extracted with EtOAc (2×). The combined organic layers were washed with brine, dried using a phase separator cartridge, and concentrated under reduced pressure to afford tert-butyl(4S)-5-amino-4-[5-[4-[(R)-[3-[(2S)-2-(tert-butoxycarbonylamino)-3,3,3-trifluoro-propoxy]-2-chloro-phenyl]-phenyl-methyl]piperidine-1-carbonyl]-6-methyl-1-oxo-isoindolin-2-yl]-5-oxo-pentanoate as a white solid (165 mg, 100% p., 23% y.). m/z [M+Na]⁺=893.2, ¹H NMR (400 MHz, DMSO-d₆): δ (ppm) 7.67-7.59 (m, 1H), 7.55 (d, J=8.6 Hz, 2H), 7.48-7.08 (m, 11H), 7.05-6.93 (m, 1H), 4.71 (dd, J=10.2, 4.0 Hz, 1H), 4.66-4.46 (m, 3H), 4.46-4.26 (m, 2H), 4.20 (t, J=7.0 Hz, 1H), 4.12-4.04 (m, 1H), 3.21 (s, 2H), 2.97 (q, J=13.1 Hz, 1H), 2.78 (d, J=14.6 Hz, 1H), 2.56 (d, J=11.6 Hz, 1H), 2.32 (d, J=5.7 Hz, 2H), 2.24 (s, 2H), 2.14 (d, J=3.8 Hz, 3H), 1.95 (d, J=9.9 Hz, 1H), 1.42-1.30 (m, 18H), [α]_{D}=+23° mL·dm⁻¹ g⁻¹[20° C., Na lamp, 589 nm, C=3.3 mg/mL, EtOH].

### Example 78

To a stirred solution of tert-butyl(4S)-5-amino-4-[5-[4-[(R)-[3-[(2S)-2-(tert-butoxycarbonylamino)-3,3,3-trifluoro-propoxy]-2-chloro-phenyl]-phenyl-methyl]piperidine-1-carbonyl]-6-methyl-1-oxo-isoindolin-2-yl]-5-oxo-pentanoate (165 mg, 0.189 mmol) in acetonitrile (1.5 mL) at ambient temperature was added 4-methylbenzenesulfonic acid hydrate (180 mg, 0.947 mmol). The reaction mixture was stirred at 80° C. for 3 h. The reaction mixture was basified with a saturated aqueous solution of NaHCO₃ and EtOAc was added. The layers were separated and the aqueous layer was extracted with EtOAc (2×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). Fractions containing were concentrated, basified with a saturated aqueous solution of NaHCO₃, and extracted with EtOAc (2×). The combined organic layers were washed with brine, dried over Na₂SO₄, filtered, and concentrated under reduced pressure. The residue was solubilized in a minimum of acetone and added dropwise to n-pentane. The resulting precipitate was filtered, washed with n-pentane, and dried under reduced pressure at 50° C. for 12 h to afford (3S)-3-[5-[4-[(R)-[3-[(2S)-2-amino-3,3,3-trifluoro-propoxy]-2-chloro-phenyl]-phenyl-methyl]piperidine-1-carbonyl]-6-methyl-1-oxo-isoindolin-2-yl]piperidine-2,6-dione as a white powder (36.4 mg, 97.1% p., 27% y.). m/z [M+H]⁺=697.4, ¹H NMR (600 MHz, DMSO-d₆): δ (ppm) 11.14-10.65 (m, 1H), 7.64-7.57 (m, 1H), 7.48-7.20 (m, 7H), 7.20-7.09 (m, 1H), 7.07-6.97 (m, 1H), 5.15-5.03 (m, 1H), 4.57-4.46 (m, 1H), 4.45-4.15 (m, 6H), 3.27-2.70 (m, 4H), 2.62-2.53 (m, 2H), 2.24 (s, 4H), 2.05-1.92 (m, 1H), 1.63-1.24 (m, 2H), 1.16-0.90 (m, 2H), NH₂ is not highlighted, [α]_{D}=+9° mL·dm⁻¹ g⁻¹[20° C., Na lamp, 589 nm, C=1.4 mg/mL, DMSO].

### Example 79

### (3S)-3-[5-[4-[(R)-[3-[((1r,3r)-3-aminocyclobutyl)amino]-2-chloro-phenyl]-phenyl-methyl]piperidine-1-carbonyl]-6-methyl-1-oxo-isoindolin-2-yl]piperidine-2,6-dione

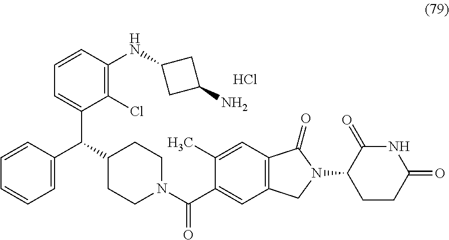 [SMILES: Cc1cc2c(cc1C(=O)N1CCC([C@H](c3ccccc3)c3cccc(N[C@H]4C[C@H](N)C4)c3Cl)CC1)CN([C@H]1CCC(=O)NC1=O)C2=O ; Cl]

### Intermediate 79A benzyl 4-[(R)-[3-[[(1r,3r)-3-(tert-butoxycarbonylamino)cyclobutyl]amino]-2-chloro-phenyl]-phenyl-methyl]piperidine-1-carboxylate

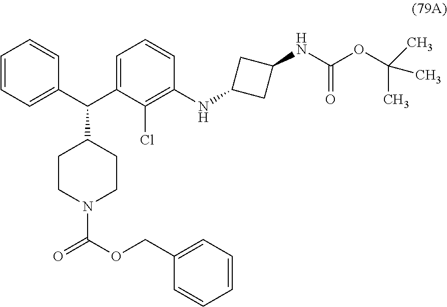 [SMILES: CC(C)(C)OC(=O)N[C@H]1C[C@H](Nc2cccc([C@@H](c3ccccc3)C3CCN(C(=O)OCc4ccccc4)CC3)c2Cl)C1]

To a stirred solution of benzyl 4-[(R)-(3-bromo-2-chloro-phenyl)-phenyl-methyl]piperidine-1-carboxylate (200 mg, 0.381 mmol) in dry 1,4-dioxane (2.5 mL) were added under argon, carbamic acid, (trans-3-aminocyclobutyl)-, 1,1-dimethylethyl ester (146 mg, 0.762 mmol), Cs₂CO₃ (380 mg, 1.14 mmol), and [1-(2-diphenylphosphanyl-1-naphthyl)-2-naphthyl]-diphenyl-phosphane (24.5 mg, 0.0381 mmol). The reaction vessel was purged with argon 10 min after which Pd(OAc)₂(10.4 mg, 0.0457 mmol) was added. The reaction mixture was stirred at 100° C. overnight and at ambient temperature for 5 days. The suspension was filtered through glass fiber filter and rinsed with EtOAc. Water was added to the filtrate, the layers were separated, and the aqueous layer was extracted with EtOAc (2×). The combined organic layers were washed with brine, dried using a phase separator cartridge, and concentrated under reduced pressure. The crude was purified by flash chromatography on silica gel using a gradient of EtOAc in n-heptane from 5% to 50% to afford benzyl 4-[(R)-[3-[[(1r,3r)-3-(tert-butoxycarbonylamino)cyclobutyl]amino]-2-chloro-phenyl]-phenyl-methyl]piperidine-1-carboxylate as an off-white foam (200 mg, 98% p., 84% y.). m/z [M-tBu+2H]⁺=548.0, ¹H NMR (400 MHz, DMSO-d₆): δ (ppm) 7.40-7.29 (m, 7H), 7.25 (q, J=7.9 Hz, 3H), 7.19-7.08 (m, 2H), 6.93 (d, J=7.7 Hz, 1H), 6.28 (dd, J=8.3, 1.3 Hz, 1H), 5.27 (d, J=5.2 Hz, 1H), 5.05 (s, 2H), 4.14-4.05 (m, 1H), 3.95 (d, J=13.2 Hz, 2H), 3.84 (q, J=6.1 Hz, 1H), 2.77 (s, 3H), 2.41 (d, J=11.3 Hz, 1H), 2.28-2.13 (m, 4H), 1.53 (d, J=12.9 Hz, 1H), 1.37 (s, 10H), 1.09-0.90 (m, 2H).

### Intermediate 79B: tert-butyl N-[(1r,3r)-3-[2-chloro-3-[(R)-phenyl(4-piperidyl)methyl]anilino]cyclobutyl]carbamate

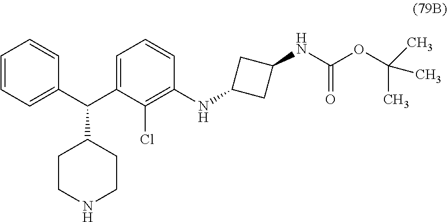 [SMILES: CC(C)(C)OC(=O)N[C@H]1C[C@H](Nc2cccc([C@@H](c3ccccc3)C3CCNCC3)c2Cl)C1]

To a stirred solution of benzyl 4-[(R)-[3-[[(1r,3r)-3-(tert-butoxycarbonylamino) cyclobutyl]amino]-2-chloro-phenyl]-phenyl-methyl]piperidine-1-carboxylate (200 mg, 0.324 mmol) in EtOH (1.5 mL) at ambient temperature was added palladium on carbon (10% w/w, 34.5 mg, 0.0324 mmol). The reaction mixture was stirred under H₂(g) (0.3 bars) for 16 h at ambient temperature. The reaction mixture was filtered through a pad of talcum powder. The filtrate was concentrated under reduced pressure to afford tert-butyl N-[(1r,3r)-3-[2-chloro-3-[(R)-phenyl(4-piperidyl)methyl]anilino]cyclobutyl]carbamate as a colorless gum (149 mg, 88% p., 86% y.). m/z [M+H]⁺=470.4.

### Intermediate 79C: tert-butyl(4S)-5-amino-4-[5-[4-[(R)-[3-[[(1r,3r)-3-(tert-butoxycarbonylamino)cyclobutyl]amino]-2-chloro-phenyl]-phenyl-methyl]piperidine-1-carbonyl]-6-methyl-1-oxo-isoindolin-2-yl]-5-oxo-pentanoate

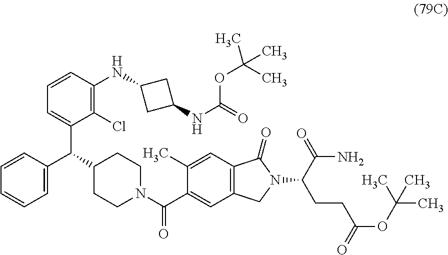 [SMILES: Cc1cc2c(cc1C(=O)N1CCC([C@H](c3ccccc3)c3cccc(N[C@H]4C[C@H](NC(=O)OC(C)(C)C)C4)c3Cl)CC1)CN([C@@H](CCC(=O)OC(C)(C)C)C(N)=O)C2=O]

To a stirred solution of 2-[(1S)-4-tert-butoxy-1-carbamoyl-4-oxo-butyl]-6-methyl-1-oxo-isoindoline-5-carboxylic acid (134 mg, 0.349 mmol) in dry DCM (2.0 mL) at ambient temperature were added DIPEA (166 μL, 0.951 mmol), tert-butyl N-[(1r,3r)-3-[2-chloro-3-[(R)-phenyl(4-piperidyl)methyl]anilino]cyclobutyl]carbamate (149 mg, 0.317 mmol), and a solution of T3P in DCM (53% w/w, 283 μL, 0.475 mmol). The reaction mixture was stirred at ambient temperature for 20 min. The reaction was quenched with the addition of a saturated aqueous solution of NH₄Cl. DCM was added to the mixture. The layers were separated and the aqueous layer was extracted with DCM. The combined organic layers were washed with brine, dried over Na₂SO₄, filtered, and concentrated under reduced pressure. The crude was purified by flash chromatography on silica gel using a gradient of EtOAc/EtOH (3/1) in n-heptane from 10% to 100% to afford tert-butyl(4S)-5-amino-4-[5-[4-[(R)-[3-[(1r,3r)-3-(tert-butoxycarbonylamino)cyclobutyl]amino]-2-chloro-phenyl]-phenyl-methyl]piperidine-1-carbonyl]-6-methyl-1-oxo-isoindolin-2-yl]-5-oxo-pentanoate as a white solid (85.0 mg, 98% p., 32% y.). m/z [M+Na]⁺=850.3.

### Example 79

To a stirred solution of tert-butyl(4S)-5-amino-4-[5-[4-[(R)-[3-[(1r,3r)-3-(tert-butoxycarbonylamino)cyclobutyl]amino]-2-chloro-phenyl]-phenyl-methyl]piperidine-1-carbonyl]-6-methyl-1-oxo-isoindolin-2-yl]-5-oxo-pentanoate (85.0 mg, 0.101 mmol) in acetonitrile (1.0 mL) at ambient temperature was added 4-methylbenzenesulfonic acid hydrate (95.6 mg, 0.503 mmol). The reaction mixture was stirred at 80° C. for 3 h. The reaction mixture was basified with a saturated aqueous solution of NaHCO₃ and EtOAc was added. The layers were separated, and the aqueous layer extracted with EtOAc (2×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated and basified with a saturated aqueous solution of NaHCO₃. The aqueous layer was extracted with EtOAc (2×). The combined organic layers were washed with brine, dried over Na₂SO₄, filtered, and concentrated under reduced pressure. The residue was solubilized in THF (1.0 mL) at ambient temperature and a 2 M solution of HCl in Et₂O(503 μL, 1.01 mmol) was added dropwise. The mixture was stirred at ambient temperature for 10 min and then Et₂O was added. The resulting precipitate was triturated for 30 min, filtered, washed with Et₂O, and dried under reduced pressure at 50° C. for 12 h to afford (3S)-3-[5-[4-[(R)-[3-[((1r,3r)-3-aminocyclobutyl)amino]-2-chloro-phenyl]-phenyl-methyl]piperidine-1-carbonyl]-6-methyl-1-oxo-isoindolin-2-yl]piperidine-2,6-dione hydrochloride as a white powder (25.7 mg, 93.3% p., 35% y.). m/z [M+H]⁺=654.4, ¹H NMR (500 MHz, DMSO-d₆): δ (ppm) 10.98 (s, 1H), 8.19-7.97 (m, 3H), 7.66-7.56 (m, 1H), 7.51-6.84 (m, 8H), 6.35-6.21 (m, 1H), 5.61-5.35 (m, 1H), 5.18-5.01 (m, 1H), 4.68-3.95 (m, 5H), 3.74 (dt, J=12.8, 5.1 Hz, 1H), 3.26-2.53 (m, 6H), 2.46-2.20 (m, 9H), 2.11-1.87 (m, 1H), 1.70-1.34 (m, 2H), 1.20-1.00 (m, 1H), [α]_{D}=+21° mL·dm⁻¹·g⁻¹[20° C., Na lamp, 589 nm, C=1.5 mg/mL, DMSO].

### Example 80

### (3S)-3-[5-[4-[(R)-[3-[(1S)-2-amino-1-methyl-ethoxy]-2-chloro-phenyl]-phenyl-methyl]piperidine-1-carbonyl]-6-methyl-1-oxo-isoindolin-2-yl]piperidine-2,6-dione hydrochloride

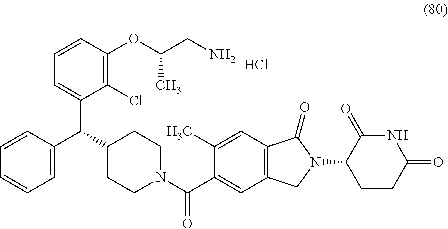 [SMILES: Cc1cc2c(cc1C(=O)N1CCC([C@H](c3ccccc3)c3cccc(O[C@@H](C)CN)c3Cl)CC1)CN([C@H]1CCC(=O)NC1=O)C2=O ; Cl]

### Intermediate 80A: tert-butyl(4S)-5-amino-4-[5-[4-[(R)-[3-[(1S)-2-(tert-butoxycarbonylamino)-1-methyl-ethoxy]-2-chloro-phenyl]-phenyl-methyl]piperidine-1-carbonyl]-6-methyl-1-oxo-isoindolin-2-yl]-5-oxo-pentanoate

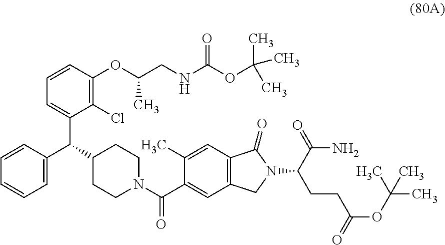 [SMILES: Cc1cc2c(cc1C(=O)N1CCC([C@H](c3ccccc3)c3cccc(O[C@@H](C)CNC(=O)OC(C)(C)C)c3Cl)CC1)CN([C@@H](CCC(=O)OC(C)(C)C)C(N)=O)C2=O]

In a vial, to a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.211 mmol) in dry THF (1.4 mL) were added successively tert-butyl [(2R)-2-hydroxypropyl]carbamate (115 mg, 0.634 mmol), triphenylphosphane (166 mg, 0.634 mmol), and then DBAD (150 mg, 0.634 mmol). The reaction mixture was stirred at ambient temperature for 24 h. The reaction mixture was concentrated under reduced pressure. The crude was purified by flash chromatography on silica gel using a gradient of EtOAc/EtOH (3/1) in n-heptane from 5% to 100% to afford tert-butyl(4S)-5-amino-4-[5-[4-[(R)-[3-[(1S)-2-(tert-butoxycarbonylamino)-1-methyl-ethoxy]-2-chloro-phenyl]-phenyl-methyl]piperidine-1-carbonyl]-6-methyl-1-oxo-isoindolin-2-yl]-5-oxo-pentanoate as a white solid (170 mg, 97% p., 95% y.). m/z [M-Boc+2H]⁺=717.4. [α]_{D}=+72° mL·dm⁻¹ g⁻¹[20° C., Na lamp, 589 nm, C=1.5 mg/mL, EtOH].

### Example 80

In a sealed vial, to a solution of tert-butyl(4S)-5-amino-4-[5-[4-[(R)-[3-[(1S)-2-(tert-butoxycarbonylamino)-1-methyl-ethoxy]-2-chloro-phenyl]-phenyl-methyl]piperidine-1-carbonyl]-6-methyl-1-oxo-isoindolin-2-yl]-5-oxo-pentanoate (170 mg, 0.202 mmol) in acetonitrile (3.4 mL) was added 4-methylbenzenesulfonic acid hydrate (230 mg, 1.21 mmol). The solution was stirred at 90° C. for 2 h. The mixture was diluted with EtOAc and washed with a saturated aqueous solution of NaHCO₃ and brine. The organic layer was dried over Na₂SO₄, filtered, and concentrated under reduced pressure. The crude was purified by reverse-phase preparative chromatography using a gradient of acetonitrile in water from 0% to 100% (0.1% AcOH). The desired fractions were combined and concentrated under reduced pressure until acetonitrile evaporated. A saturated aqueous solution of NaHCO₃ was added. The mixture was extracted with EtOAc (2×). The combined organic layers were dried over Na₂SO₄, filtered, and concentrated under reduced pressure. The residue was solubilized in Et₂O(5.0 mL) and treated with a 4 M solution of HCl in 1,4-dioxane (151 μL). The mixture was stirred at ambient temperature for 30 min. The resulting suspension was filtered and dried under at 50° C. under reduced pressure for 48 hours to afford (3S)-3-[5-[4-[(R)-[3-[(1S)-2-amino-1-methyl-ethoxy]-2-chloro-phenyl]-phenyl-methyl]piperidine-1-carbonyl]-6-methyl-1-oxo-isoindolin-2-yl]piperidine-2,6-dione hydrochloride as a white powder (31.5 mg, 99.6% p., 23% y.). m/z [M+H]⁺=643.4, ¹H NMR (600 MHz, DMSO-d₆): δ (ppm) 10.99-10.95 (m, 1H), 8.11-7.95 (m, 3H), 7.62-7.59 (m, 1H), 7.49-7.22 (m, 7H), 7.21-7.11 (m, 1H), 7.11-7.01 (m, 1H), 5.13-5.06 (m, 1H), 4.72-4.64 (m, 1H), 4.56-4.16 (m, 4H), 3.27-2.70 (m, 6H), 2.62-2.52 (m, 2H), 2.42-2.30 (m, 2H), 2.24 (br d, J=3.7 Hz, 2H), 2.02-1.95 (m, 1H), 1.64-1.23 (m, 2H), 1.21-1.16 (m, 3H), 1.14-0.90 (m, 2H), [α]_{D}=+18° mL·dm⁻¹·g^{-1 [20}° C., Na lamp, 589 nm, C=1.3 mg/mL, DMSO].

### Example 81

### (3S)-3-[5-[4-[(R)-[3-[(2R)-2-amino-3,3-difluoro-propoxy]-2-chloro-phenyl]-phenyl-methyl]piperidine-1-carbonyl]-6-methyl-1-oxo-isoindolin-2-yl]piperidine-2,6-dione hydrochloride

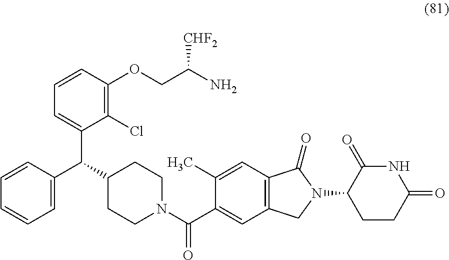 [SMILES: Cc1cc2c(cc1C(=O)N1CCC([C@H](c3ccccc3)c3cccc(OC[C@@H](N)C(F)F)c3Cl)CC1)CN([C@H]1CCC(=O)NC1=O)C2=O]

### Intermediate 81A: tert-butyl(4S)-5-amino-4-[5-[4-[(R)-[3-[(2R)-2-(tert-butoxycarbonylamino)-3,3-difluoro-propoxy]-2-chloro-phenyl]-phenyl-methyl]piperidine-1-carbonyl]-6-methyl-1-oxo-isoindolin-2-yl]-5-oxo-pentanoate

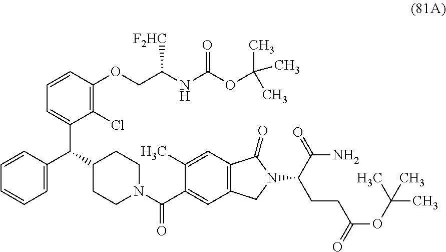 [SMILES: Cc1cc2c(cc1C(=O)N1CCC([C@H](c3ccccc3)c3cccc(OC[C@@H](NC(=O)OC(C)(C)C)C(F)F)c3Cl)CC1)CN([C@@H](CCC(=O)OC(C)(C)C)C(N)=O)C2=O]

To a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (400 mg, 0.606 mmol) and tert-butyl N-[(2S)-1,1-difluoro-3-hydroxypropan-2-yl]carbamate (256 mg, 1.21 mmol) in THF (6.1 mL) were added successively triphenylphosphane (318 mg, 1.21 mmol) and then DBAD (279 mg, 1.21 mmol). The reaction mixture was stirred at ambient temperature overnight. The reaction mixture was concentrated under reduced pressure. The crude was purified by flash chromatography on silica gel using a gradient of EtOAc/EtOH (3/1) in n-heptane from 10% to 100% to afford tert-butyl(4S)-5-amino-4-[5-[4-[(R)-[3-[(2R)-2-(tert-butoxycarbonylamino)-3,3-difluoro-propoxy]-2-chloro-phenyl]-phenyl-methyl]piperidine-1-carbonyl]-6-methyl-1-oxo-isoindolin-2-yl]-5-oxo-pentanoate as an off-white solid (431 mg, 66% p., 55% y.). m/z [M+H]⁺=853.7.

### Example 81

To a stirred solution of tert-butyl(4S)-5-amino-4-[5-[4-[(R)-[3-[(2R)-2-(tert-butoxycarbonylamino)-3,3-difluoro-propoxy]-2-chloro-phenyl]-phenyl-methyl]piperidine-1-carbonyl]-6-methyl-1-oxo-isoindolin-2-yl]-5-oxo-pentanoate (430 mg, 0.504 mmol) in acetonitrile (5.0 mL) was added 4-methylbenzenesulfonic acid hydrate (958 mg, 5.04 mmol). The reaction mixture was stirred at 90° C. for 4 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). Acetonitrile was removed under reduced pressure. The aqueous layer was basified with a saturated aqueous solution of NaHCO₃. The aqueous layer was extracted with EtOAc (3×). The combined organic layers were washed with brine, dried over

Na₂SO₄, filtered, and concentrated under reduced pressure. The residue was solubilized in 1,4-dioxane (13 mL). Next, a 4 M solution of HCl in 1,4-dioxane (1.3 mL) was added dropwise. The reaction mixture was stirred for 30 min. Et₂O(139 mL) was added dropwise. The precipitate was triturated for 1 h, filtered, washed with Et₂O, and dried under reduced pressure at 50° C. overnight to afford (3S)-3-[5-[4-[(R)-[3-[(2R)-2-amino-3,3-difluoro-propoxy]-2-chloro-phenyl]-phenyl-methyl]piperidine-1-carbonyl]-6-methyl-1-oxo-isoindolin-2-yl]piperidine-2,6-dione hydrochloride as a white solid (117 mg, 98.7% p., 32% y.). m/z [M+H]⁺=679.4, ¹H NMR (500 MHz, DMSO-d₆): δ (ppm) 11.07-10.88 (m, 1H), 8.95-8.41 (m, 3H), 7.62 (br d, J=7.9 Hz, 1H), 7.50-7.42 (m, 1H), 7.41-7.39 (m, 1H), 7.39-7.37 (m, 1H), 7.35 (br s, 2H), 7.30 (s, 2H), 7.22-7.11 (m, 1H), 7.07 (dd, J=8.1, 1.4 Hz, 1H), 6.43 (br dd, J=13.6, 3.1 Hz, 1H), 5.21-4.93 (m, 1H), 4.62-4.47 (m, 1H), 4.47-4.33 (m, 2H), 4.33-4.27 (m, 1H), 4.27-4.22 (m, 1H), 4.22-4.17 (m, 1H), 4.16-4.02 (m, 1H), 3.28-3.16 (m, 1H), 3.09-2.95 (m, 1H), 2.94-2.84 (m, 1H), 2.83-2.68 (m, 1H), 2.65-2.52 (m, 2H), 2.45-2.36 (m, 1H), 2.35-2.20 (m, 3H), 2.06-1.93 (m, 1H), 1.67-1.27 (m, 2H), 1.25-1.12 (m, 1H), 1.07-0.90 (m, 1H), [α]_{D}=+4° mL·dm⁻¹ g⁻¹[20° C., Na lamp, 589 nm, C=1.4 mg/mL, DMSO].

### Example 82

### (3S)-3-[5-[4-[rel-(S)-[3-(2-aminoethoxy)-2-fluoro-phenyl]-phenyl-methyl]piperidine-1-carbonyl]-6-methyl-1-oxo-isoindolin-2-yl]piperidine-2,6-dione hydrochloride

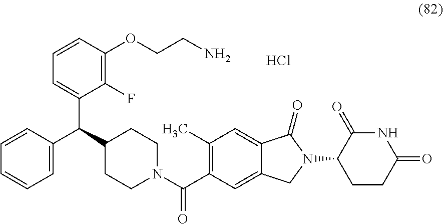 [SMILES: Cc1cc2c(cc1C(=O)N1CCC([C@@H](c3ccccc3)c3cccc(OCCN)c3F)CC1)CN([C@H]1CCC(=O)NC1=O)C2=O ; Cl]

### Intermediate 82A: tert-butyl(4S)-5-amino-4-[5-[4-[rel-(S)-[3-[2-(tert-butoxycarbonylamino) ethoxy]-2-fluoro-phenyl]-phenyl-methyl]piperidine-1-carbonyl]-6-methyl-1-oxo-isoindolin-2-yl]-5-oxo-pentanoate

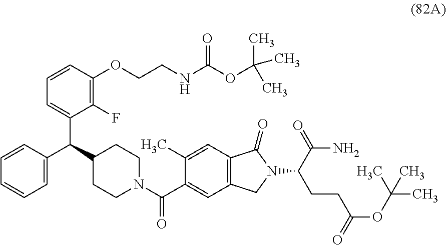 [SMILES: Cc1cc2c(cc1C(=O)N1CCC([C@@H](c3ccccc3)c3cccc(OCCNC(=O)OC(C)(C)C)c3F)CC1)CN([C@@H](CCC(=O)OC(C)(C)C)C(N)=O)C2=O]

To a stirred solution of tert-butyl(4S)-5-amino-4-[5-[4-[rel-(S)-(2-fluoro-3-hydroxy-phenyl)-phenyl-methyl]piperidine-1-carbonyl]-6-methyl-1-oxo-isoindolin-2-yl]-5-oxo-pentanoate (150 mg, 0.233 mmol) and tert-butyl(2-hydroxyethyl) carbamate (180 μL, 1.17 mmol) in THF (2.3 mL) were added triphenylphosphane (306 mg, 1.17 mmol) and then DBAD (268 mg, 1.17 mmol). The reaction mixture was stirred at ambient temperature overnight. The reaction mixture was concentrated under reduced pressure. The crude was purified by flash chromatography on silica gel using a gradient of EtOAc/EtOH (3/1) in n-heptane from 10% to 100% to afford tert-butyl(4S)-5-amino-4-[5-[4-[rel-(S)-[3-[2-(tert-butoxycarbonylamino) ethoxy]-2-fluoro-phenyl]-phenyl-methyl]piperidine-1-carbonyl]-6-methyl-1-oxo-isoindolin-2-yl]-5-oxo-pentanoate as an off-white solid (104 mg, 35% p., 20% y.). m/z [M+H]⁺=853.7.

### Example 82

To a stirred solution of tert-butyl(4S)-5-amino-4-[5-[4-[rel-(S)-[3-[2-(tert-butoxycarbonylamino) ethoxy]-2-fluoro-phenyl]-phenyl-methyl]piperidine-1-carbonyl]-6-methyl-1-oxo-isoindolin-2-yl]-5-oxo-pentanoate (104 mg, 0.132 mmol) in acetonitrile (1.3 mL) was added 4-methylbenzenesulfonic acid hydrate (251 mg, 1.32 mmol). The reaction mixture was stirred at 80° C. for 4 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). Acetonitrile was removed under reduced pressure. The aqueous layer was basified with a saturated aqueous solution of NaHCO₃. The aqueous layer was extracted with EtOAc (3×). The combined organic layers were washed with brine, dried over Na₂SO₄, filtered, and concentrated under reduced pressure. The residue was solubilized in 1,4-dioxane (4.9 mL). Next, a 4 M solution of HCl in 1,4-dioxane (490 μL) was added dropwise. The reaction mixture was stirred for 30 min. Et₂O(54 mL) was added dropwise. The precipitate was triturated for 1 h, filtered, washed with Et₂O, and dried under reduced pressure at 50° C. overnight to afford (3S)-3-[5-[4-[rel-(S)-[3-(2-aminoethoxy)-2-fluoro-phenyl]-phenyl-methyl]piperidine-1-carbonyl]-6-methyl-1-oxo-isoindolin-2-yl]piperidine-2,6-dione hydrochloride as a white solid (7.80 mg, 99.6% p., 9% y.). m/z [M+H]⁺=613.4, ¹H NMR (600 MHz, DMSO-d₆): δ (ppm) 11.10-10.84 (m, 1H), 7.95 (br s, 3H), 7.64-7.56 (m, 1H), 7.48-7.04 (m, 8H), 7.03-6.92 (m, 1H), 5.18-5.00 (m, 1H), 4.62-4.48 (m, 1H), 4.46-4.34 (m, 1H), 4.33-4.23 (m, 1H), 4.21-4.09 (m, 2H), 3.93 (br d, J=11.0 Hz, 1H), 3.27-3.14 (m, 3H), 3.08-2.93 (m, 1H), 2.93-2.84 (m, 1H), 2.83-2.71 (m, 1H), 2.65-2.52 (m, 2H), 2.43-2.21 (m, 4H), 2.04-1.94 (m, 1H), 1.68-0.88 (m, 4H).

### Example 83

### (3S)-3-[5-[4-[(R)-[3-(3-amino-2,2-difluoro-propoxy)-2-chloro-phenyl]-phenyl-methyl]piperidine-1-carbonyl]-6-methyl-1-oxo-isoindolin-2-yl]piperidine-2,6-dione

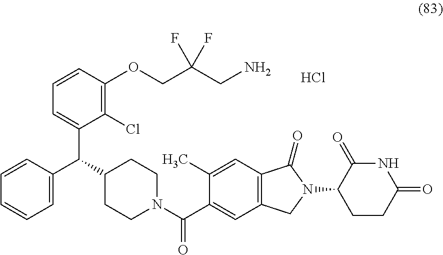 [SMILES: Cc1cc2c(cc1C(=O)N1CCC([C@H](c3ccccc3)c3cccc(OCC(F)(F)CN)c3Cl)CC1)CN([C@H]1CCC(=O)NC1=O)C2=O ; Cl]

### Intermediate 83A: benzyl 4-[(R)-[3-[3-(tert-butoxycarbonylamino)-2,2-difluoro-propoxy]-2-chloro-phenyl]-phenyl-methyl]piperidine-1-carboxylate

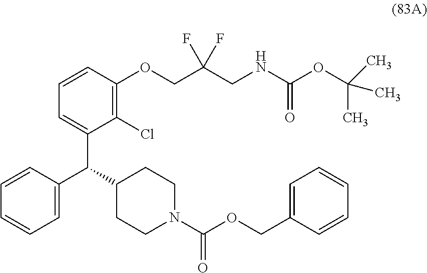 [SMILES: CC(C)(C)OC(=O)NCC(F)(F)COc1cccc([C@@H](c2ccccc2)C2CCN(C(=O)OCc3ccccc3)CC2)c1Cl]

A vial was successively charged with benzyl 4-[(R)-(3-bromo-2-chloro-phenyl)-phenyl-methyl]piperidine-1-carboxylate (250 mg, 0.486 mmol), tert-butyl N-(2,2-difluoro-3-hydroxypropyl) carbamate (529 mg, 2.43 mmol), Ir[dF(CF₃)ppy]₂(dtbbpy)PF₆ (5.5 mg, 0.0486 mmol) and 4,4′-di-tert-butyl-2,2′-bipyridine dichloronickel (10.2 mg, 0.0243 mmol) in EtOAc (5.1 mL). The reaction mixture was degassed with argon for 10 min. Next, 2,2,6,6-tetramethylpiperidine (82 μL, 0.486 mmol) was added and the reaction mixture was irradiated with a blue LEDs (456 nm) without ventilation overnight. Additional Ir[dF(CF₃)ppy]₂(dtbbpy)PF₆ (5.5 mg, 0.0486 mmol) and 4,4′-di-tert-butyl-2,2′-bipyridine dichloronickel (10.2 mg, 0.024 mmol) were added and the reaction mixture was degassed with argon for 5 min. Next, 2,2,6,6-tetramethylpiperidine (82 μL, 0.486 mmol) was added and the reaction mixture was irradiated with a blue LEDs (456 nm) without ventilation overnight. The reaction was quenched with the addition of a saturated aqueous solution of NH₄Cl. EtOAc was added to the mixture. The layers were separated, and the aqueous layer was extracted with EtOAc (2×). The combined organic layers were washed with brine, dried using hydrophobic paper and concentrated under reduced pressure. The crude was purified by flash chromatography on silica gel using a gradient of EtOAc in n-heptane from 0% to 100% to afford expected product benzyl 4-[(R)-[3-[3-(tert-butoxycarbonylamino)-2,2-difluoro-propoxy]-2-chloro-phenyl]-phenyl-methyl]piperidine-1-carboxylate as a yellow oil (709 mg, 73% p., quant. yield)). m/z [M+Na]⁺=651.3.

### Intermediate 83B: tert-butyl N-[3-[2-chloro-3-[(R)-phenyl(4-piperidyl)methyl]phenoxy]-2,2-difluoro-propyl]carbamate

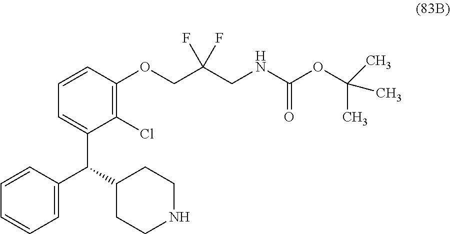 [SMILES: CC(C)(C)OC(=O)NCC(F)(F)COc1cccc([C@@H](c2ccccc2)C2CCNCC2)c1Cl]

To a solution of benzyl 4-[(R)-[3-[3-(tert-butoxycarbonylamino)-2,2-difluoro-propoxy]-2-chloro-phenyl]-phenyl-methyl]piperidine-1-carboxylate (709 mg, 1.13 mmol) in EtOH (17 mL) was added palladium on carbon (10% w/w, 180 mg, 0.169 mmol). The reaction mixture was stirred under H₂(g) (0.3 bars) overnight at ambient temperature. The suspension was filtered, washed with EtOH, and dried under reduced pressure to afford tert-butyl N-[3-[2-chloro-3-[(R)-phenyl(4-piperidyl)methyl]phenoxy]-2,2-difluoro-propyl]carbamate as a yellow oil (624 mg, 80% p., 89% y.). m/z [M+H]⁺=495.3.

### Intermediate 83C: tert-butyl(4S)-5-amino-4-[5-[4-[(R)-[3-[3-(tert-butoxycarbonylamino)-2,2-difluoro-propoxy]-2-chloro-phenyl]-phenyl-methyl]piperidine-1-carbonyl]-6-methyl-1-oxo-isoindolin-2-yl]-5-oxo-pentanoate

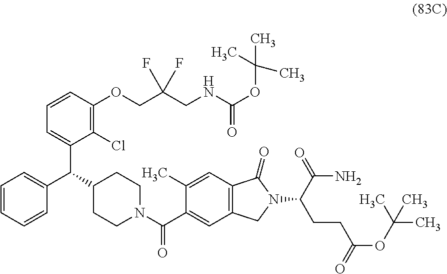 [SMILES: Cc1cc2c(cc1C(=O)N1CCC([C@H](c3ccccc3)c3cccc(OCC(F)(F)CNC(=O)OC(C)(C)C)c3Cl)CC1)CN([C@@H](CCC(=O)OC(C)(C)C)C(N)=O)C2=O]

To a stirred suspension of 2-[(1S)-4-tert-butoxy-1-carbamoyl-4-oxo-butyl]-6-methyl-1-oxo-isoindoline-5-carboxylic acid (420 mg, 1.06 mmol) and tert-butyl N-[3-[2-chloro-3-[(R)-phenyl(4-piperidyl)methyl]phenoxy]-2,2-difluoro-propyl]carbamate (624 mg, 1.01 mmol) in DMF (8.9 mL) were added successively DIPEA (705 μL, 4.03 mmol) and HATU (593 mg, 1.51 mmol). The reaction mixture was stirred at ambient temperature 2 h. The reaction was quenched with the addition of a saturated aqueous solution of NH₄Cl. The aqueous layer was extracted with EtOAc (2×). The combined organic layers were washed with a saturated aqueous solution of NaHCO₃ and brine, dried over hydrophobic paper, and concentrated under reduced pressure. The crude was purified by flash chromatography on silica gel using a gradient of EtOAc/EtOH (3/1) in n-heptane from 2% to 100%. The resulting residue was purified by reverse-phase preparative chromatography using a gradient of acetonitrile in water from 0% to 100% (0.1% AcOH). Acetonitrile was removed under reduced pressure. The solution was basified with a saturated aqueous solution of NaHCO₃ and EtOAc was added. The layers were separated and the aqueous layer was extracted with EtOAc (2×). The combined organic layers were washed with brine, dried using a phase separator cartridge, and concentrated under reduced pressure to afford tert-butyl(4S)-5-amino-4-[5-[4-[(R)-[3-[3-(tert-butoxycarbonylamino)-2,2-difluoro-propoxy]-2-chloro-phenyl]-phenyl-methyl]piperidine-1-carbonyl]-6-methyl-1-oxo-isoindolin-2-yl]-5-oxo-pentanoate as a white foam (168 mg, 92% p., 18% y.). m/z [M+Na]⁺=875.3. [α]_{D}=+13° mL·dm⁻¹ g⁻¹[20° C., Na lamp, 589 nm, C=1.7 mg/mL, EtOH].

### Example 83

To a stirred solution of tert-butyl(4S)-5-amino-4-[5-[4-[(R)-[3-[3-(tert-butoxycarbonylamino)-2,2-difluoro-propoxy]-2-chloro-phenyl]-phenyl-methyl]piperidine-1-carbonyl]-6-methyl-1-oxo-isoindolin-2-yl]-5-oxo-pentanoate (165 mg, 0.178 mmol) in acetonitrile (1.8 mL) was added 4-methylbenzenesulfonic acid hydrate (169 mg, 0.889 mmol). The reaction mixture was stirred at 80° C. for 3 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure. The solid was solubilized in 1,4-dioxane (4.8 mL) and a 4 M solution of HCl in 1,4-dioxane (480 μL) was added. The reaction mixture was stirred at ambient temperature for 30 min and Et₂O(48 mL) was added. After 1 h, the precipitate was filtered, washed with Et₂O, and dried under reduced pressure overnight to afford (3S)-3-[5-[4-[(R)-[3-(3-amino-2,2-difluoro-propoxy)-2-chloro-phenyl]-phenyl-methyl]piperidine-1-carbonyl]-6-methyl-1-oxo-isoindolin-2-yl]piperidine-2,6-dione hydrochloride as a white powder (63.0 mg, 99.2% p., 49% y.). m/z [M+H]⁺=679.4, ¹H NMR (600 MHz, DMSO-d₆): δ (ppm) 10.97 (s, 1H), 8.77-8.05 (m, 3H), 7.64-7.57 (m, 1H), 7.46 (s, 1H), 7.37 (br d, J=8.1 Hz, 2H), 7.36-7.31 (m, 2H), 7.29 (s, 2H), 7.21-7.09 (m, 1H), 7.08-6.96 (m, 1H), 5.09 (td, J=8.8, 4.6 Hz, 1H), 4.60-4.44 (m, 3H), 4.43-4.35 (m, 1H), 4.35-4.26 (m, 1H), 4.19 (br d, J=11.3 Hz, 1H), 3.63-3.57 (m, 1H), 3.56-3.47 (m, 1H), 3.27-3.14 (m, 1H), 3.06-2.94 (m, 1H), 2.94-2.84 (m, 1H), 2.77 (br d, J=1.0 Hz, 1H), 2.64-2.52 (m, 2H), 2.44-2.36 (m, 1H), 2.35-2.18 (m, 3H), 2.07-1.92 (m, 1H), 1.68-1.21 (m, 2H), 1.20-0.78 (m, 2H).

### Example 84

### (3S)-3-[5-[4-[rel-(R)-(2-chlorophenyl)-(3-pyridyl)methyl]piperidine-1-carbonyl]-6-methyl-1-oxo-isoindolin-2-yl]piperidine-2,6-dione hydrochloride

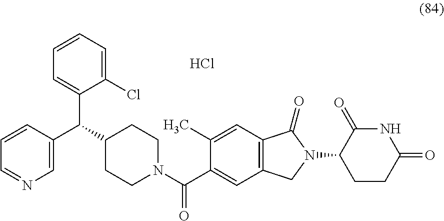 [SMILES: Cc1cc2c(cc1C(=O)N1CCC([C@H](c3cccnc3)c3ccccc3Cl)CC1)CN([C@H]1CCC(=O)NC1=O)C2=O ; Cl]

### Intermediate 84A: benzyl 4-[(2-chlorophenyl)-hydroxy-(3-pyridyl)methyl]piperidine-1-carboxylate

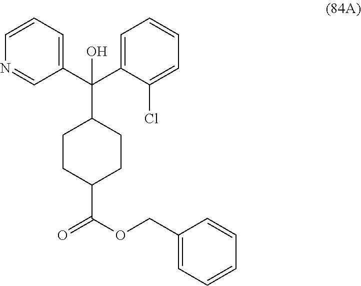 [SMILES: O=C(OCc1ccccc1)C1CCC(C(O)(c2cccnc2)c2ccccc2Cl)CC1]

To a stirred solution of 3-bromopyridine (331 μL, 3.35 mmol) and benzyl 4-(2-chlorobenzoyl) piperidine-1-carboxylate (1.00 g, 2.79 mmol) in dry THF (19 mL) at −78° C. under argon was slowly added 2.5 M n-butyllithium (1.7 mL, 4.19 mmol). The reaction mixture was stirred at −78° C. for 30 min and then at ambient temperature for 1 h. The reaction was quenched with the addition of a saturated aqueous solution of NH₄Cl and water. EtOAc were added to the mixture. The layers were separated and the aqueous layer was extracted with EtOAc (2×). The combined organic layers were washed with brine, dried using a phase separator cartridge and concentrated under reduced pressure. The crude was purified by flash chromatography on silica gel using a gradient of EtOAc in cyclohexane from 5% to 50%. The desired fractions were concentrated under reduced pressure to afford (benzyl 4-[(2-chlorophenyl)-hydroxy-(3-pyridyl)methyl]piperidine-1-carboxylate as a white foam (734 mg, 96% p., 58% y.). m/z [M+H]⁺=437.3, ¹H NMR (400 MHz, DMSO-d₆): δ (ppm) 8.58 (d, J=2.0 Hz, 1H), 8.37 (dd, J=4.7, 1.6 Hz, 1H), 8.03 (d, J=7.8 Hz, 1H), 7.76 (d, J=8.0 Hz, 1H), 7.45-7.23 (m, 9H), 5.05 (s, 2H), 4.03 (q, J=7.1 Hz, 2H), 3.19 (d, J=11.4 Hz, 1H), 2.90 (d, J=58.6 Hz, 2H), 1.47-1.24 (m, 3H), 1.24-1.13 (m, 2H).

### Intermediate 84B: benzyl 4-[chloro-(2-chlorophenyl)-(3-pyridyl)methyl]piperidine-1-carboxylate

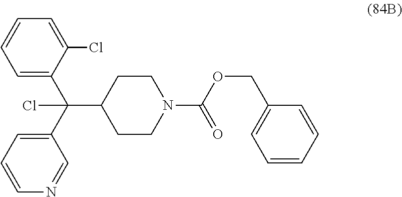 [SMILES: O=C(OCc1ccccc1)N1CCC(C(Cl)(c2cccnc2)c2ccccc2Cl)CC1]

To a stirred solution of benzyl 4-[(2-chlorophenyl)-hydroxy-(3-pyridyl)methyl]piperidine-1-carboxylate (734 mg, 1.61 mmol) in dry THF (10 mL) at 0° C. under nitrogen was added pyridine (457 μL, 5.64 mmol) dropwise. The solution was stirred at this temperature for 30 min. Thionyl chloride (867 μL, 11.3 mmol) was added dropwise and the reaction mixture was stirred at 0° C. for 2 h. The reaction was quenched with the addition of a saturated aqueous solution of NH₄Cl. EtOAc was added to the mixture. The layers were separated, and the aqueous layer was extracted with EtOAc (2×). The combined organic layers were washed with brine, dried using a phase separator cartridge, and concentrated under reduced pressure. The crude was purified by flash chromatography on silica gel using a gradient of MeOH in DCM from 1% to 5% to afford benzyl 4-[chloro-(2-chlorophenyl)-(3-pyridyl)methyl]piperidine-1-carboxylate as a white foam (412 mg, 72% p., 40% y.). m/z [M+H]⁺=455.8.

### Intermediate 84C: benzyl 4-[rel-(R)-(2-chlorophenyl)-(3-pyridyl)methyl]piperidine-1-carboxylate

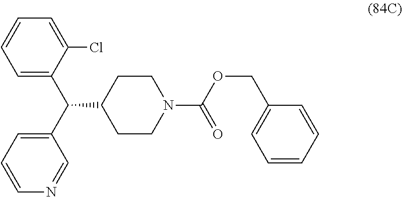 [SMILES: O=C(OCc1ccccc1)N1CCC([C@H](c2cccnc2)c2ccccc2Cl)CC1]

A sealed vial was charged with a solution of benzyl 4-[chloro-(2-chlorophenyl)-(3-pyridyl)methyl]piperidine-1-carboxylate (413 mg, 0.653 mmol) in AcOH (6.5 mL). zinc dust (171 mg, 2.61 mmol) was added. The reaction mixture was stirred at 100° C. for 45 min. The reaction mixture was filtered. The reaction was quenched with the addition of an aqueous saturated aqueous solution of NaHCO₃(pH=10). EtOAc was added. The layers were separated, and the aqueous layer was extracted with EtOAc (2×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). Acetonitrile was removed under reduced pressure. The aqueous layer was basified with a saturated aqueous solution of NaHCO₃ and EtOAc was added. The layers were separated and the aqueous layer was extracted with EtOAc (2×). The combined organic layers were washed with brine, dried using a phase separator cartridge, and concentrated under reduced pressure to afford benzyl 4-[(2-chlorophenyl)-(3-pyridyl)methyl]piperidine-1-carboxylate as a colorless oil (130 mg, 97% p., 46% y.). The product was purified by chiral supercritical chromatography (Waters Prep SFC200, Chiralpak IC (5 μm, 250×4.6 mm), CO₂/MeOH 70/30 as mobile phase, flow rate 2.4 mL/min, 104 bars) to afford (second isomer eluted) as a colorless oil (benzyl 4-[rel-(R)-(2-chlorophenyl)-(3-pyridyl)methyl]piperidine-1-carboxylate (40.0 mg, 100% p., 15% y., d.e.>96.4%)). m/z [M+H]⁺=421.3, ¹H NMR (400 MHz, DMSO-d₆): δ (ppm) 8.57 (d, J=1.9 Hz, 1H), 8.39 (dd, J=4.7, 1.6 Hz, 1H), 7.77 (td, J=7.8, 1.7 Hz, 2H), 7.40 (dd, J=7.9, 1.3 Hz, 1H), 7.38-7.31 (m, 6H), 7.31-7.18 (m, 2H), 5.05 (s, 2H), 4.15 (d, J=11.3 Hz, 1H), 3.98 (d, J=12.0 Hz, 2H), 2.79 (s, 2H), 2.51 (d, J=1.8 Hz, 3H), 1.50 (d, J=12.9 Hz, 1H), 1.31 (d, J=11.6 Hz, 1H), 1.05 (dqd, J=36.8, 12.7, 4.4 Hz, 2H).

### Intermediate 84D: 3-[rel-(R)-(2-chlorophenyl)-(4-piperidyl)methyl]pyridine dihydroiodide

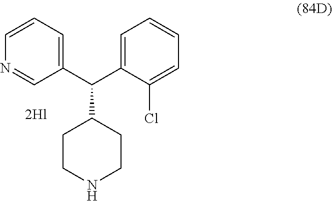 [SMILES: Clc1ccccc1[C@@H](c1cccnc1)C1CCNCC1]

To a stirred solution of benzyl 4-[rel-(R)-(2-chlorophenyl)-(3-pyridyl)methyl]piperidine-1-carboxylate (40.0 mg, 0.095 mmol) in dry acetonitrile (950 μL) at ambient temperature under nitrogen was added iodo (trimethyl) silane (41 μL, 0.285 mmol) dropwise. The reaction mixture was stirred at ambient temperature for 1 h. EtOH (46 μL, 1.14 mmol) was added dropwise. The mixture was stirred at ambient temperature for 30 min and was concentrated under reduced pressure. Et₂O was added and the resulting mixture was stirred at ambient temperature for 1 h. The solid was filtered, washed with Et₂O, and dried under reduced pressure to afford 3-[rel-(R)-(2-chlorophenyl)-(4-piperidyl)methyl]pyridine dihydroiodide as a red solid (41.0 mg, 92% p., 73% y.). m/z [M+H]⁺=287.3.

### Intermediate 84E: tert-butyl(4S)-5-amino-4-[5-[4-[rel-(R)-(2-chlorophenyl)-(3-pyridyl)methyl]piperidine-1-carbonyl]-6-methyl-1-oxo-isoindolin-2-yl]-5-oxo-pentanoate

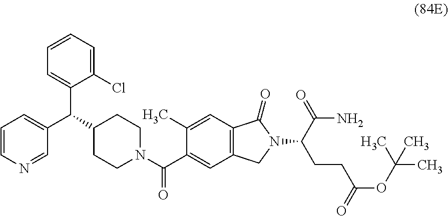 [SMILES: Cc1cc2c(cc1C(=O)N1CCC([C@H](c3cccnc3)c3ccccc3Cl)CC1)CN([C@@H](CCC(=O)OC(C)(C)C)C(N)=O)C2=O]

To a solution of 3-[rel-(R)-(2-chlorophenyl)-(4-piperidyl)methyl]pyridine dihydroiodide (41 mg, 0.076 mmol) in DMF (380 μL) were added 2-[(1S)-4-tert-butoxy-1-carbamoyl-4-oxo-butyl]-6-methyl-1-oxo-isoindoline-5-carboxylic acid (29.9 mg, 0.079 mmol), DIPEA (39.6 μL, 0.227 mmol) and HATU (44 mg, 0.113 mmol). The mixture was stirred at ambient temperature for 1 h. Water, EtOAc and FastWorkX® were added. The organic solvent was evaporated under reduced pressure. The reaction mixture was filtered through a dry-loading cartridge and the resin was rinsed with water. The crude was purified by flash chromatography on silica gel using a gradient of EtOAc/EtOH (3/1) in cyclohexane from 10% to 100% to afford tert-butyl(4S)-5-amino-4-[5-[4-[rel-(R)-(2-chlorophenyl)-(3-pyridyl)methyl]piperidine-1-carbonyl]-6-methyl-1-oxo-isoindolin-2-yl]-5-oxo-pentanoate as an orange gum (30.2 mg, 85% p., 53% y.). m/z [M+H]⁺=645.5.

### Example 84

To a stirred solution of tert-butyl(4S)-5-amino-4-[5-[4-[rel-(R)-(2-chlorophenyl)-(3-pyridyl)methyl]piperidine-1-carbonyl]-6-methyl-1-oxo-isoindolin-2-yl]-5-oxo-pentanoate (30.2 mg, 0.047 mmol) in acetonitrile (470 μL) was added 4-methylbenzenesulfonic acid hydrate (44.2 mg, 0.232 mmol). The reaction mixture was stirred at 80° C. for 3 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure. The solid was solubilized in 1,4-dioxane (1.3 mL) and a 4 M solution of HCl in 1,4-dioxane (130 μL) was added. The reaction mixture was stirred at ambient temperature for 30 min and Et₂O(13 mL) was added. After 1 h, the precipitate was filtered, washed with Et₂O, and dried under reduced pressure overnight to afford (3S)-3-[5-[4-[rel-(R)-(2-chlorophenyl)-(3-pyridyl)methyl]piperidine-1-carbonyl]-6-methyl-1-oxo-isoindolin-2-yl]piperidine-2,6-dione hydrochloride as a beige powder (4.90 mg, 97.3% p., 17% y.). m/z [M+H]⁺=571.3, ¹H NMR (600 MHz, DMSO-d₆): δ (ppm) 10.99-10.95 (m, 1H), 8.80-8.66 (m, 1H), 8.63-8.47 (m, 1H), 8.22-8.03 (m, 1H), 7.87-7.74 (m, 1H), 7.67-7.52 (m, 2H), 7.49-7.35 (m, 3H), 7.30-7.19 (m, 1H), 5.14-5.06 (m, 1H), 4.54 (br s, 1H), 4.46-4.36 (m, 1H), 4.34-4.24 (m, 2H), 3.42-3.39 (m, 1H), 3.30-3.17 (m, 1H), 3.05-2.85 (m, 2H), 2.82-2.67 (m, 2H), 2.64-2.56 (m, 1H), 2.41-2.31 (m, 2H), 2.25 (s, 2H), 2.01-1.95 (m, 1H), 1.70-1.38 (m, 1H), 1.32-0.90 (m, 3H).

### Example 85

### (3S)-3-[5-[4-[(R)-[3-(2-amino-4,4-difluoro-butoxy)-2-chloro-phenyl]-phenyl-methyl]piperidine-1-carbonyl]-6-methyl-1-oxo-isoindolin-2-yl]piperidine-2,6-dione

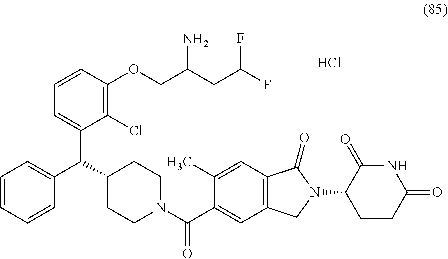

### Intermediate 85A: tert-butyl(4S)-5-amino-4-[5-[4-[(R)-[3-[2-(tert-butoxycarbonylamino)-4,4-difluoro-butoxy]-2-chloro-phenyl]-phenyl-methyl]piperidine-1-carbonyl]-6-methyl-1-oxo-isoindolin-2-yl]-5-oxo-pentanoate

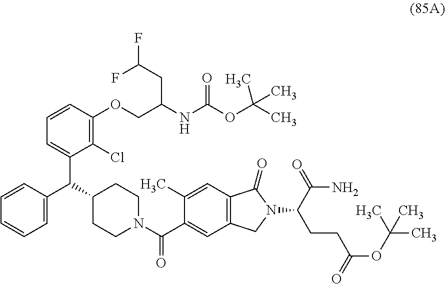

To a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (300 mg, 0.423 mmol) and tert-butyl N-(4,4-difluoro-1-hydroxybutan-2-yl) carbamate (301 mg, 1.27 mmol) in dry THF (2.5 mL) were added successively triphenylphosphane (333 mg, 1.27 mmol) and then DBAD (301 mg, 1.27 mmol). The reaction mixture was stirred at ambient temperature for 16 h. The reaction mixture was concentrated under reduced pressure. The crude was purified by flash chromatography on silica gel using a gradient of EtOAc/EtOH (3/1) (0.5% conc. NH₄OH) in n-heptane from 5% to 100% to afford tert-butyl(4S)-5-amino-4-[5-[4-[(R)-[3-[2-(tert-butoxycarbonylamino)-4,4-difluoro-butoxy]-2-chloro-phenyl]-phenyl-methyl]piperidine-1-carbonyl]-6-methyl-1-oxo-isoindolin-2-yl]-5-oxo-pentanoate as an off-white solid (307 mg, 98% p., 82% y.). m/z [M+Na]⁺=889.6, ¹H NMR (400 MHz, DMSO-d₆): δ (ppm) 7.55 (d, J=8.9 Hz, 2H), 7.49-7.08 (m, 8H), 7.08-6.88 (m, 2H), 6.32-5.89 (m, 1H), 4.71 (dd, J=10.1, 3.9 Hz, 1H), 4.64-4.32 (m, 3H), 4.24-4.17 (m, 1H), 4.01-3.81 (m, 3H), 3.27-3.14 (m, 1H), 2.97 (q, J=12.6 Hz, 1H), 2.82-2.71 (m, 1H), 2.62-2.53 (m, 1H), 2.32 (d, J=5.5 Hz, 1H), 2.24 (s, 2H), 2.20-2.05 (m, 4H), 2.02-1.87 (m, 1H), 1.56 (m, 1H), 1.40-1.25 (m, 18H), 1.26-1.20 (m, 1H), 1.17-0.91 (m, 1H).

### Example 85

To a stirred solution of tert-butyl(4S)-5-amino-4-[5-[4-[(R)-[3-[2-(tert-butoxycarbonylamino)-4,4-difluoro-butoxy]-2-chloro-phenyl]-phenyl-methyl]piperidine-1-carbonyl]-6-methyl-1-oxo-isoindolin-2-yl]-5-oxo-pentanoate (100 mg, 0.113 mmol) in acetonitrile (1.0 mL) at ambient temperature was added 4-methylbenzenesulfonic acid hydrate (107 mg, 0.565 mmol). The reaction mixture was stirred at 80° C. for 2 h. The reaction mixture was basified with a saturated aqueous solution of NaHCO₃ and EtOAc was added. The layers were separated and the aqueous layer was extracted with EtOAc (2×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated and basified with a saturated aqueous solution of NaHCO₃. The aqueous layer was extracted with EtOAc (2×). The combined organic layers were washed with brine, dried over Na₂SO₄, filtered, and concentrated under reduced pressure. The residue was solubilized in dry THF (1.0 mL) at ambient temperature and a 2 M solution of HCl in Et₂O(565 L) was added dropwise. The mixture was stirred at ambient temperature for 10 min and then Et₂O was added. The resulting precipitate was triturated for 30 min, filtered, washed with Et₂O, and dried under reduced pressure at 50° C. for 12 h to afford (3S)-3-[5-[4-[(R)-[3-(2-amino-4,4-difluoro-butoxy)-2-chloro-phenyl]-phenyl-methyl]piperidine-1-carbonyl]-6-methyl-1-oxo-isoindolin-2-yl]piperidine-2,6-dione hydrochloride as a white powder (36.0 mg, 99.1% p., 43% y.). m/z [M+H]⁺=693.4, ¹H NMR (500 MHz, DMSO-d₆): δ (ppm) 10.98 (s, 1H), 8.28 (br d, J=0.9 Hz, 3H), 7.62 (br d, J=7.6 Hz, 1H), 7.54-7.08 (m, 8H), 7.06-6.89 (m, 1H), 6.57-6.12 (m, 1H), 5.17-5.02 (m, 1H), 4.59-4.47 (m, 1H), 4.47-4.36 (m, 1H), 4.36-4.26 (m, 1H), 4.26-4.07 (m, 3H), 3.80-3.66 (m, 1H), 3.28-3.15 (m, 1H), 3.07-2.85 (m, 2H), 2.85-2.70 (m, 1H), 2.67-2.53 (m, 2H), 2.47-2.19 (m, 6H), 2.06-1.92 (m, 1H), 1.70-0.82 (m, 4H).

### Example 86

### (3S)-3-[5-[4-[(R)-[3-[(2S)-2-aminobutoxy]-2-chloro-phenyl]-phenyl-methyl]piperidine-1-carbonyl]-6-methyl-1-oxo-isoindolin-2-yl]piperidine-2,6-dione hydrochloride

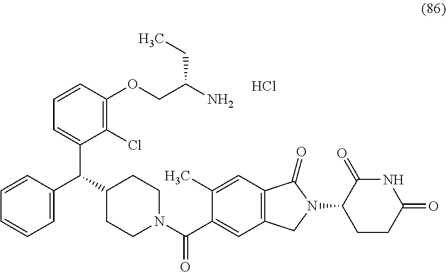 [SMILES: CC[C@H](N)COc1cccc([C@@H](c2ccccc2)C2CCN(C(=O)c3cc4c(cc3C)C(=O)N([C@H]3CCC(=O)NC3=O)C4)CC2)c1Cl ; Cl]

### Intermediate 86A: tert-butyl(4S)-5-amino-4-[5-[4-[(R)-[3-[(2S)-2-(tert-butoxycarbonylamino)butoxy]-2-chloro-phenyl]-phenyl-methyl]piperidine-1-carbonyl]-6-methyl-1-oxo-isoindolin-2-yl]-5-oxo-pentanoate

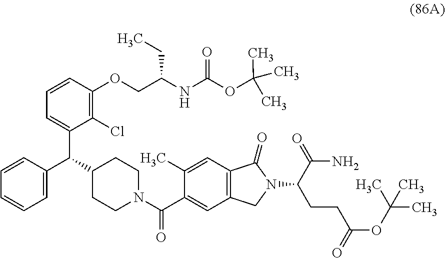 [SMILES: CC[C@@H](COc1cccc([C@@H](c2ccccc2)C2CCN(C(=O)c3cc4c(cc3C)C(=O)N([C@@H](CCC(=O)OC(C)(C)C)C(N)=O)C4)CC2)c1Cl)NC(=O)OC(C)(C)C]

To a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.211 mmol) and tert-butyl N-[(2S)-1-hydroxybutan-2-yl]carbamate (126 mg, 0.634 mmol) in dry THF (1.3 mL) were added successively triphenylphosphane (166 mg, 0.634 mmol) and then DBAD (150 mg, 0.634 mmol). The reaction mixture was stirred at ambient temperature for 16 h. The reaction mixture was concentrated under reduced pressure. The crude was purified by flash chromatography on silica gel using a gradient of EtOAc/EtOH (3/1) (0.5% conc. NH₄OH) in n-heptane from 5% to 100% to afford tert-butyl(4S)-5-amino-4-[5-[4-[(R)-[3-[(2S)-2-(tert-butoxycarbonylamino) butoxy]-2-chloro-phenyl]-phenyl-methyl]piperidine-1-carbonyl]-6-methyl-1-oxo-isoindolin-2-yl]-5-oxo-pentanoate as a white solid (147 mg, 97% p., 81% y.). m/z [M-Boc+2H]⁺=731.5, ¹H NMR (400 MHz, DMSO-d₆): δ (ppm) 7.61-7.07 (m, 12H), 6.99-6.88 (m, 1H), 6.74 (dd, J=15.2, 8.4 Hz, 1H), 4.71 (dd, J=10.2, 4.0 Hz, 1H), 4.62-4.46 (m, 2H), 4.41 (d, J=16.8 Hz, 1H), 4.24-4.17 (m, 1H), 3.95-3.88 (m, 1H), 3.88-3.75 (m, 1H), 3.73-3.56 (m, 1H), 3.28-3.14 (m, 1H), 2.97 (q, J=12.5 Hz, 1H), 2.78 (d, J=14.0 Hz, 1H), 2.56 (s, 1H), 2.32 (d, J=5.4 Hz, 1H), 2.23 (s, 2H), 2.14 (d, J=3.2 Hz, 3H), 1.98 (d, J=10.3 Hz, 1H), 1.60 (d, J=12.9 Hz, 2H), 1.51 (s, 1H), 1.44-1.20 (m, 22H), 1.20-0.93 (m, 1H), [α]_{D}=+23° mL·dm⁻¹·g⁻¹[20° C., Na lamp, 589 nm, C=1.6 mg/mL, DMSO].

### Example 86

To a stirred solution of tert-butyl(4S)-5-amino-4-[5-[4-[(R)-[3-[(2S)-2-(tert-butoxycarbonylamino)butoxy]-2-chloro-phenyl]-phenyl-methyl]piperidine-1-carbonyl]-6-methyl-1-oxo-isoindolin-2-yl]-5-oxo-pentanoate (147 mg, 0.171 mmol) in acetonitrile (1.5 mL) at ambient temperature was added 4-methylbenzenesulfonic acid hydrate (163 mg, 0.857 mmol). The reaction mixture was stirred at 80° C. for 2 h. The reaction mixture was basified with a saturated aqueous solution of NaHCO₃ and EtOAc was added. The layers were separated and the aqueous layer was extracted with EtOAc (2×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated and basified with a saturated aqueous solution of NaHCO₃. The aqueous layer was extracted with EtOAc (2×). The combined organic layers were washed with brine, dried over Na₂SO₄, filtered, and concentrated under reduced pressure. The residue was solubilized in dry THF (1.5 mL) at ambient temperature and a 2 M solution of HCl in Et₂O(85.7 μL) was added dropwise. The mixture was stirred at ambient temperature for 10 min and then Et₂O was added. The resulting precipitate was triturated for 30 min, filtered, washed with Et₂O, and dried under reduced pressure at 50° C. for 12 h to afford (3S)-3-[5-[4-[(R)-[3-[(2S)-2-aminobutoxy]-2-chloro-phenyl]-phenyl-methyl]piperidine-1-carbonyl]-6-methyl-1-oxo-isoindolin-2-yl]piperidine-2,6-dione hydrochloride as a white powder (50.2 mg, 99.2% p., 42% y.). m/z [M+H]⁺=657.5, ¹H NMR (500 MHz, DMSO-d₆): δ (ppm) 11.10-10.77 (m, 1H), 8.05 (br d, J=0.9 Hz, 3H), 7.66-7.58 (m, 1H), 7.49-7.40 (m, 1H), 7.39-7.38 (m, 1H), 7.37-7.33 (m, 2H), 7.33-7.30 (m, 1H), 7.29 (s, 2H), 7.21-7.09 (m, 1H), 7.03 (dd, J=8.0, 1.5 Hz, 1H), 5.16-5.04 (m, 1H), 4.60-4.47 (m, 1H), 4.46-4.36 (m, 1H), 4.35-4.26 (m, 1H), 4.25-4.17 (m, 1H), 4.17-4.05 (m, 2H), 3.51-3.36 (m, 1H), 3.27-3.16 (m, 1H), 3.07-2.95 (m, 1H), 2.94-2.84 (m, 1H), 2.83-2.70 (m, 1H), 2.66-2.53 (m, 2H), 2.44-2.35 (m, 1H), 2.35-2.22 (m, 3H), 2.05-1.93 (m, 1H), 1.80-1.74 (m, 1H), 1.73-1.65 (m, 1H), 1.65-1.25 (m, 2H), 1.20-1.01 (m, 2H), 1.00-0.92 (m, 3H), [α]_{D}=+6° mL·dm⁻¹ g⁻¹[20° C., Na lamp, 589 nm, C=1.6 mg/mL, DMSO].

### Example 87

### (3S)-3-[5-[4-[(R)-[3-[(2R)-2-amino-3,3,3-trifluoro-propoxy]-2-chloro-phenyl]-phenyl-methyl]piperidine-1-carbonyl]-6-methyl-1-oxo-isoindolin-2-yl]piperidine-2,6-dione

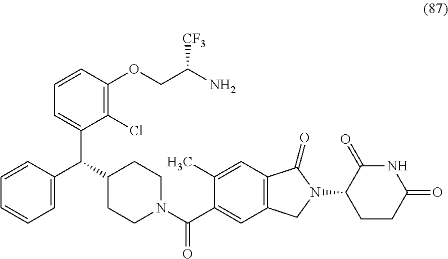 [SMILES: Cc1cc2c(cc1C(=O)N1CCC([C@H](c3ccccc3)c3cccc(OC[C@@H](N)C(F)(F)F)c3Cl)CC1)CN([C@H]1CCC(=O)NC1=O)C2=O]

### Intermediate 87A: benzyl 4-[(R)-[3-[(2R)-2-(tert-butoxycarbonylamino)-3,3,3-trifluoro-propoxy]-2-chloro-phenyl]-phenyl-methyl]piperidine-1-carboxylate

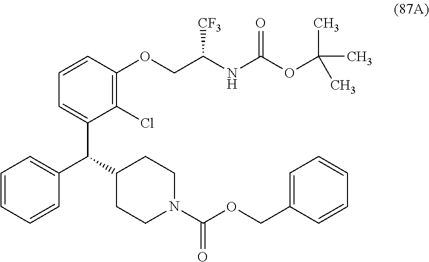 [SMILES: CC(C)(C)OC(=O)N[C@H](COc1cccc([C@@H](c2ccccc2)C2CCN(C(=O)OCc3ccccc3)CC2)c1Cl)C(F)(F)F]

A vial was successively charged with benzyl 4-[(R)-(3-bromo-2-chloro-phenyl)-phenyl-methyl]piperidine-1-carboxylate (250 mg, 0.486 mmol), tert-butyl N-[(2R)-1,1,1-trifluoro-3-hydroxypropan-2-yl]carbamate (124 mg, 0.514 mmol), Ir[dF(CF₃)ppy]₂(dtbbpy)PF₆ (5.5 mg, 0.00486 mmol), and 4,4′-di-tert-butyl-2,2′-bipyridine dichloronickel (10.2 mg, 0.0243 mmol) in EtOAc (5.1 mL). The reaction mixture was degassed with argon for 10 min. Next, 2,2,6,6-tetramethylpiperidine (82 μL, 0.486 mmol) was added and the reaction mixture was irradiated with a blue LEDs (456 nm) without ventilation overnight. Additional Ir[dF(CF₃)ppy]₂(dtbbpy)PF₆ (5.5 mg, 0.00486 mmol) and 4,4′-di-tert-butyl-2,2′-bipyridine dichloronickel (10.2 mg, 0.0243 mmol) were added and the reaction mixture was degassed with argon for 5 min. Next, 2,2,6,6-tetramethylpiperidine (82 μL, 0.486 mmol) was added and the reaction mixture was irradiated with a blue LEDs (456 nm) without ventilation overnight. Additional Ir[dF(CF₃)ppy]₂(dtbbpy)PF₆ (5.5 mg, 0.00486 mmol) and 4,4′-di-tert-butyl-2,2′-bipyridine dichloronickel (10.2 mg, 0.0243 mmol) were added and the reaction mixture was degassed with argon for 5 min. Further 2,2,6,6-tetramethylpiperidine (82 μL, 0.486 mmol) was added and the reaction mixture was irradiated with a blue LEDs (456 nm) without ventilation (temperature increased until 31° C.) overnight. Additional Ir[dF(CF₃)ppy]₂(dtbbpy)PF₆ (11.1 mg, 0.00972 mmol), 4,4′-di-tert-butyl-2,2′-bipyridine dichloronickel (19.9 mg, 0.486 mmol) were added and the reaction mixture was degassed with argon for 5 min. Again, additional 2,2,6,6-tetramethylpiperidine (164 μL, 0.972 mmol) was added and the reaction mixture was irradiated with a blue LEDs (456 nm) without ventilation overnight. The reaction was quenched with the addition of a saturated aqueous solution of NH₄Cl. EtOAc was added. The layers were separated and the aqueous layer was extracted with EtOAc (2×). The combined organic layers were washed with brine, dried using hydrophobic paper, and concentrated under reduced pressure. The crude was purified by reverse-phase preparative chromatography using a gradient of acetonitrile in water from 0% to 100% (0.1% AcOH). Acetonitrile was removed under reduced pressure. The solution was basified with a saturated aqueous solution of NaHCO₃ and EtOAc was added. The layers were separated and the aqueous layer was extracted with EtOAc (2×). The combined organic layers were washed with brine, dried using a phase separator cartridge, and concentrated under reduced pressure to afford benzyl 4-[(R)-[3-[(2R)-2-(tert-butoxycarbonylamino)-3,3,3-trifluoro-propoxy]-2-chloro-phenyl]-phenyl-methyl]piperidine-1-carboxylate as a colorless gum (117 mg, 90% p., 33% y.). m/z [M+Na]⁺=669.3, ¹H NMR (400 MHz, DMSO-d₆): δ (ppm) 7.67 (d, J=9.1 Hz, 1H), 7.40-7.22 (m, 11H), 7.20-7.11 (m, 1H), 6.99 (dd, J=7.3, 2.2 Hz, 1H), 5.05 (s, 2H), 4.68-4.54 (m, 1H), 4.28 (dd, J=10.0, 4.6 Hz, 1H), 4.22-4.08 (m, 2H), 3.96 (d, J=13.1 Hz, 2H), 2.72 (d, J=44.0 Hz, 3H), 2.46 (s, 1H), 1.41 (s, 9H), 1.26 (d, J=19.7 Hz, 1H), 1.02 (dt, J=28.4, 12.7 Hz, 2H).

### Intermediate 87B: tert-butyl N-[(1R)-1-[[2-chloro-3-[(R)-phenyl(4-piperidyl)methyl]phenoxy]methyl]-2,2,2-trifluoro-ethyl]carbamate

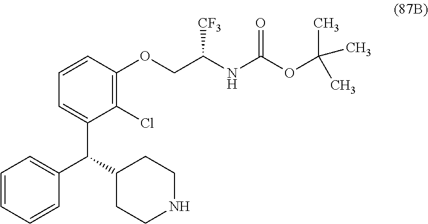 [SMILES: CC(C)(C)OC(=O)N[C@H](COc1cccc([C@@H](c2ccccc2)C2CCNCC2)c1Cl)C(F)(F)F]

To a solution of benzyl 4-[(R)-[3-[(2R)-2-(tert-butoxycarbonylamino)-3,3,3-trifluoro-propoxy]-2-chloro-phenyl]-phenyl-methyl]piperidine-1-carboxylate (116 mg, 0.161 mmol) in EtOH (2.5 mL) was added palladium on carbon (10% w/w, 25.8 mg, 0.0242 mmol). The reaction mixture was stirred under H₂(g) (0.3 bars) overnight at ambient temperature. The suspension was filtered, washed with EtOH, and dried under reduced pressure to afford tert-butyl N-[(1R)-1-[2-chloro-3-[(R)-phenyl(4-piperidyl)methyl]phenoxy]methyl]-2,2,2-trifluoro-ethyl]carbamate as a yellow foam (82.0 mg, 75% p., 74% y.). m/z [M+H]⁺=513.3, ¹H NMR (400 MHz, DMSO-d₆): δ (ppm) 7.67 (d, J=9.1 Hz, 1H), 7.39-7.31 (m, 2H), 7.31-7.22 (m, 4H), 7.19-7.09 (m, 1H), 6.98 (q, J=4.4 Hz, 1H), 4.67-4.53 (m, 1H), 4.28 (dd, J=10.0, 4.6 Hz, 1H), 4.19-4.07 (m, 2H), 2.93-2.85 (m, 2H), 2.49-2.39 (m, 1H), 2.39-2.21 (m, 1H), 1.41 (s, 9H), 1.37-1.23 (m, 4H), 1.05-0.90 (m, 2H).

### Intermediate 87C: tert-butyl(4S)-5-amino-4-[5-[4-[(R)-[3-[(2R)-2-(tert-butoxycarbonylamino)-3,3,3-trifluoro-propoxy]-2-chloro-phenyl]-phenyl-methyl]piperidine-1-carbonyl]-6-methyl-1-oxo-isoindolin-2-yl]-5-oxo-pentanoate

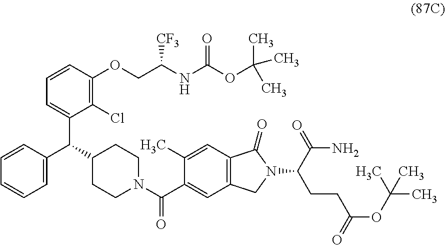 [SMILES: Cc1cc2c(cc1C(=O)N1CCC([C@H](c3ccccc3)c3cccc(OC[C@@H](NC(=O)OC(C)(C)C)C(F)(F)F)c3Cl)CC1)CN([C@@H](CCC(=O)OC(C)(C)C)C(N)=O)C2=O]

To a stirred suspension of 2-[(1S)-4-tert-butoxy-1-carbamoyl-4-oxo-butyl]-6-methyl-1-oxo-isoindoline-5-carboxylic acid (49.9 mg, 0.126 mmol) and tert-butyl N-[(1R)-1-[2-chloro-3-[(R)-phenyl(4-piperidyl)methyl]phenoxy]methyl]-2,2,2-trifluoro-ethyl]carbamate (82 mg, 0.120 mmol) in DMF (1.1 mL) were added successively DIPEA (83.8 μL, 0.480 mmol) and HATU (70.5 mg, 0.180 mmol). The reaction mixture was stirred at ambient temperature 2 h. The reaction was quenched with the addition of a saturated aqueous solution of NH₄Cl. The aqueous layer was extracted with EtOAc (2×). The combined organic layers were washed with a saturated aqueous solution of NaHCO₃ and brine, dried over hydrophobic paper, and concentrated under reduced pressure. The crude was purified by flash chromatography on silica gel using a gradient of EtOAc/EtOH (3/1) in n-heptane from 2% to 100% to afford tert-butyl(4S)-5-amino-4-[5-[4-[(R)-[3-[(2R)-2-(tert-butoxycarbonylamino)-3,3,3-trifluoro-propoxy]-2-chloro-phenyl]-phenyl-methyl]piperidine-1-carbonyl]-6-methyl-1-oxo-isoindolin-2-yl]-5-oxo-pentanoate as an orange foam (93.6 mg, 90% p., 81% y.). m/z [M+Na]⁺=893.5. [α]_{D}=+6° mL·dm⁻¹·g⁻¹[20° C., Na lamp, 589 nm, C=1.4 mg/mL, EtOH].

### Example 87

To a stirred solution of tert-butyl(4S)-5-amino-4-[5-[4-[(R)-[3-[(2R)-2-(tert-butoxycarbonylamino)-3,3,3-trifluoro-propoxy]-2-chloro-phenyl]-phenyl-methyl]piperidine-1-carbonyl]-6-methyl-1-oxo-isoindolin-2-yl]-5-oxo-pentanoate (94.0 mg, 0.0971 mmol) in dry acetonitrile (1.0 mL) was added 4-methylbenzenesulfonic acid hydrate (240 mg, 1.26 mmol). The reaction mixture was stirred at 90° C. for 2.5 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure until acetonitrile evaporated. A saturated aqueous solution of NaHCO₃ was added. The resulting precipitate was filtered, washed with water, and dried under reduced pressure overnight to afford (3S)-3-[5-[4-[(R)-[3-[(2R)-2-amino-3,3,3-trifluoro-propoxy]-2-chloro-phenyl]-phenyl-methyl]piperidine-1-carbonyl]-6-methyl-1-oxo-isoindolin-2-yl]piperidine-2,6-dione as a white powder (19.3 mg, 97.9% p., 28% y.). m/z [M+H]⁺=697.4, ¹H NMR (500 MHz, DMSO-d₆): δ (ppm) 10.97 (br s, 1H), 7.68-7.53 (m, 1H), 7.52-7.08 (m, 8H), 7.05-6.91 (m, 1H), 5.21-4.99 (m, 1H), 4.61-4.00 (m, 6H), 3.79-3.61 (m, 1H), 3.25-3.15 (m, 1H), 3.08-2.68 (m, 3H), 2.57 (br s, 2H), 2.43-2.30 (m, 2H), 2.25 (br s, 2H), 2.13 (br s, 2H), 2.06-1.92 (m, 1H), 1.70-0.82 (m, 4H), [α]_{D}=−5° mL·dm⁻¹·g^{-1 [20}° C., Na lamp, 589 nm, C=1.0 mg/mL, DMSO].

### Example 88

### (3S)-3-[5-[4-[(R)-[2-chloro-3-(isoxazol-4-ylmethoxy)phenyl]-phenyl-methyl]piperidine-1-carbonyl]-6-methyl-1-oxo-isoindolin-2-yl]piperidine-2,6-dione

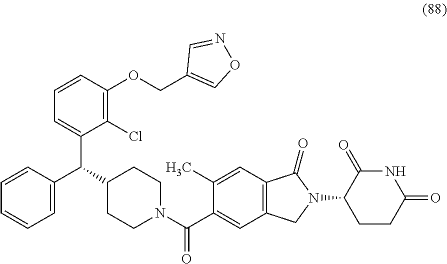 [SMILES: Cc1cc2c(cc1C(=O)N1CCC([C@H](c3ccccc3)c3cccc(OCc4cnoc4)c3Cl)CC1)CN([C@H]1CCC(=O)NC1=O)C2=O]

### Intermediate 88A: tert-butyl(4S)-5-amino-4-[5-[4-[(R)-[2-chloro-3-(isoxazol-4-ylmethoxy)phenyl]-phenyl-methyl]piperidine-1-carbonyl]-6-methyl-1-oxo-isoindolin-2-yl]-5-oxo-pentanoate

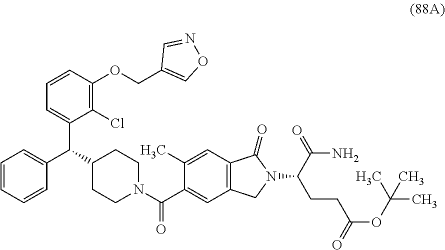 [SMILES: Cc1cc2c(cc1C(=O)N1CCC([C@H](c3ccccc3)c3cccc(OCc4cnoc4)c3Cl)CC1)CN([C@@H](CCC(=O)OC(C)(C)C)C(N)=O)C2=O]

In a sealed vial, to a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.211 mmol) in dry THF (1.4 mL) were added (1,2-oxazol-4-yl)methanol (66.1 mg, 0.634 mmol), triphenylphosphane (166 mg, 0.634 mmol) and then DBAD (150 mg, 0.634 mmol). The reaction mixture was stirred at ambient temperature for 72 h. The reaction mixture was concentrated under reduced pressure. The crude was purified by flash chromatography on silica gel using a gradient of EtOAc/EtOH (3/1) in n-heptane from 5% to 100% to afford tert-butyl(4S)-5-amino-4-[5-[4-[(R)-[2-chloro-3-(isoxazol-4-ylmethoxy)phenyl]-phenyl-methyl]piperidine-1-carbonyl]-6-methyl-1-oxo-isoindolin-2-yl]-5-oxo-pentanoate as a white solid (148 mg, 94% p., 89% y.). m/z [M+H]⁺=741.4. [α]_{D}=+25° mL·dm⁻¹ g⁻¹[20° C., Na lamp, 589 nm, C=2.0 mg/mL, EtOH].

### Example 88

To a stirred solution of tert-butyl(4S)-5-amino-4-[5-[4-[(R)-[2-chloro-3-(isoxazol-4-ylmethoxy)phenyl]-phenyl-methyl]piperidine-1-carbonyl]-6-methyl-1-oxo-isoindolin-2-yl]-5-oxo-pentanoate (140 mg, 0.189 mmol) in acetonitrile (1.9 mL) was added 4-methylbenzenesulfonic acid hydrate (180 mg, 0.944 mmol). The reaction mixture was stirred at 80° C. for 5 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure until most of the acetonitrile evaporated. The resulting aqueous suspension was basified with a saturated aqueous solution of NaHCO₃.

The resulting suspension was filtered and dried under at 50° C. under reduced pressure for 48 hours to afford (3S)-3-[5-[4-[(R)-[2-chloro-3-(isoxazol-4-ylmethoxy)phenyl]-phenyl-methyl]piperidine-1-carbonyl]-6-methyl-1-oxo-isoindolin-2-yl]piperidine-2,6-dione as a white powder (46.2 mg, 97.4% p., 36% y.). m/z [M+H]⁺=667.3, ¹H NMR (600 MHz, DMSO-d₆): δ (ppm) 10.97 (s, 1H), 9.13-8.98 (m, 1H), 8.75-8.65 (m, 1H), 7.62-7.57 (m, 1H), 7.52-7.16 (m, 7H), 7.15-7.03 (m, 2H), 5.11-4.98 (m, 3H), 4.58-4.13 (m, 4H), 3.27-3.15 (m, 1H), 3.01-2.85 (m, 2H), 2.81-2.71 (m, 1H), 2.61-2.52 (m, 2H), 2.41-2.30 (m, 2H), 2.24 (s, 2H), 2.04-1.90 (m, 1H), 1.65-0.90 (m, 4H), [α]_{D}=+15° mL·dm⁻¹·g⁻¹[20° C., Na lamp, 589 nm, C=1.4 mg/mL, DMSO].

### Example 89

### (3S)-3-[5-[4-[(R)-[2-chloro-3-(isoxazol-5-ylmethoxy)phenyl]-phenyl-methyl]piperidine-1-carbonyl]-6-methyl-1-oxo-isoindolin-2-yl]piperidine-2,6-dione

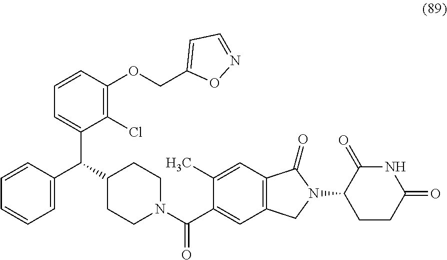 [SMILES: Cc1cc2c(cc1C(=O)N1CCC([C@H](c3ccccc3)c3cccc(OCc4ccno4)c3Cl)CC1)CN([C@H]1CCC(=O)NC1=O)C2=O]

### Intermediate 89A: tert-butyl(4S)-5-amino-4-[5-[4-[(R)-[2-chloro-3-(isoxazol-5-ylmethoxy)phenyl]-phenyl-methyl]piperidine-1-carbonyl]-6-methyl-1-oxo-isoindolin-2-yl]-5-oxo-pentanoate

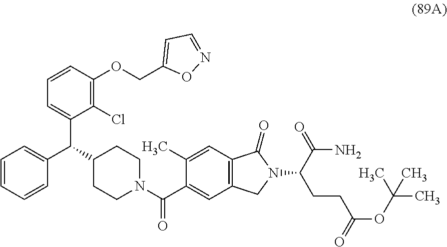 [SMILES: Cc1cc2c(cc1C(=O)N1CCC([C@H](c3ccccc3)c3cccc(OCc4ccno4)c3Cl)CC1)CN([C@@H](CCC(=O)OC(C)(C)C)C(N)=O)C2=O]

In a sealed vial, to a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.211 mmol) in dry THF (1.4 mL) were added (1,2-oxazol-5-yl)methanol (64.8 mg, 0.634 mmol), triphenylphosphane (166 mg, 0.634 mmol) and then DBAD (150 mg, 0.634 mmol). The reaction mixture was stirred at ambient temperature for 72 h. The reaction mixture was concentrated under reduced pressure. The crude was purified by flash chromatography on silica gel using a gradient of EtOAc/EtOH (3/1) in n-heptane from 5% to 100% to afford tert-butyl(4S)-5-amino-4-[5-[4-[(R)-[2-chloro-3-(isoxazol-5-ylmethoxy)phenyl]-phenyl-methyl]piperidine-1-carbonyl]-6-methyl-1-oxo-isoindolin-2-yl]-5-oxo-pentanoate as an off-white foam (145 mg, 95% p., 88% y.). m/z [M+H]⁺=741.5. [α]_{D}=+25° mL·dm⁻¹ g⁻¹[20° C., Na lamp, 589 nm, C=1.4 mg/mL, EtOH].

### Example 89

To a stirred solution of tert-butyl(4S)-5-amino-4-[5-[4-[(R)-[2-chloro-3-(isoxazol-5-ylmethoxy)phenyl]-phenyl-methyl]piperidine-1-carbonyl]-6-methyl-1-oxo-isoindolin-2-yl]-5-oxo-pentanoate (140 mg, 0.189 mmol) in acetonitrile (1.9 mL) was added 4-methylbenzenesulfonic acid hydrate (180 mg, 0.944 mmol). The reaction mixture was stirred at 80° C. for 5 h. The reaction mixture was cooled to ambient temperature and quenched with a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure until most of the acetonitrile evaporated. The resulting aqueous suspension was basified with a saturated aqueous solution of NaHCO₃. The resulting suspension was filtered and dried under at 50° C. under reduced pressure for 48 h to afford (3S)-3-[5-[4-[(R)-[2-chloro-3-(isoxazol-5-ylmethoxy)phenyl]-phenyl-methyl]piperidine-1-carbonyl]-6-methyl-1-oxo-isoindolin-2-yl]piperidine-2,6-dione as a white powder (57.9 mg, 99.0% p., 45% y.). m/z [M+H]⁺=667.4, ¹H NMR (600 MHz, DMSO-d₆): δ (ppm) 11.17-10.74 (m, 1H), 8.64-8.51 (m, 1H), 7.64-7.55 (m, 1H), 7.54-7.20 (m, 7H), 7.21-7.05 (m, 2H), 6.67-6.56 (m, 1H), 5.42-5.25 (m, 2H), 5.15-5.03 (m, 1H), 4.58-4.16 (m, 4H), 3.28-3.16 (m, 1H), 3.03-2.70 (m, 3H), 2.64-2.53 (m, 2H), 2.45-2.29 (m, 2H), 2.24 (s, 2H), 2.04-1.89 (m, 1H), 1.68-0.91 (m, 4H), [α]_{D}=+18° mL·dm⁻¹ g⁻¹[20° C., Na lamp, 589 nm, C=1.5 mg/mL, DMSO].

### Example 90

### (3S)-3-[5-[4-[(R)-(2-chloro-3-morpholino-phenyl)-phenyl-methyl]piperidine-1-carbonyl]-6-methyl-1-oxo-isoindolin-2-yl]piperidine-2,6-dione

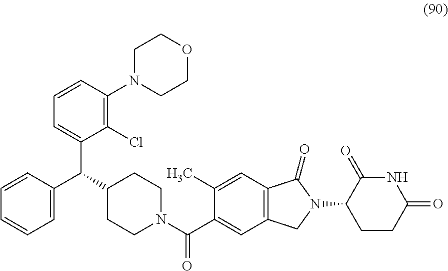 [SMILES: Cc1cc2c(cc1C(=O)N1CCC([C@H](c3ccccc3)c3cccc(N4CCOCC4)c3Cl)CC1)CN([C@H]1CCC(=O)NC1=O)C2=O]

### Intermediate 90A: benzyl 4-[(R)-(2-chloro-3-morpholino-phenyl)-phenyl-methyl]piperidine-1-carboxylate

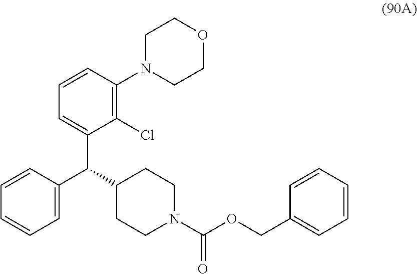 [SMILES: O=C(OCc1ccccc1)N1CCC([C@H](c2ccccc2)c2cccc(N3CCOCC3)c2Cl)CC1]

A stirred solution of benzyl 4-[(R)-(3-bromo-2-chloro-phenyl)-phenyl-methyl]piperidine-1-carboxylate (250 mg, 0.501 mmol) and morpholine (865 μL, 1.00 mmol) in 1,4-dioxane (5.0 mL) at ambient temperature was degassed with argon for 10 min. Cs₂CO₃ (500 mg, 1.50 mmol), [1-(2-diphenylphosphanyl-1-naphthyl)-2-naphthyl]-diphenyl-phosphane (64.3 mg, 0.100 mmol) and Pd(OAc)₂(11.4 mg, 0.0501 mmol) were added. The reaction mixture was stirred at 100° C. overnight. The reaction mixture was cooled to ambient temperature and poured into a saturated aqueous solution of NaHCO₃ and EtOAc. The layers were separated and the aqueous layer was extracted with EtOAc (2×). The combined organic layers were washed with brine, dried using a phase separator cartridge, and concentrated under reduced pressure. The crude was purified by flash chromatography on silica gel using a gradient of EtOAc in cyclohexane from 2% to 20% to afford benzyl 4-[(R)-(2-chloro-3-morpholino-phenyl)-phenyl-methyl]piperidine-1-carboxylate as a white powder (234 mg, 99% p., 92% y.). m/z [M+H]⁺=505.3.

### Intermediate 90B: 4-[2-chloro-3-[(R)-phenyl(4-piperidyl)methyl]phenyl]morpholine

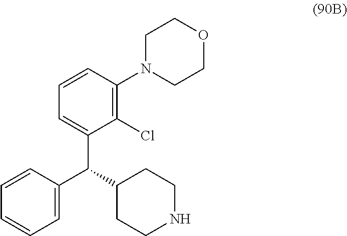 [SMILES: Clc1c([C@@H](c2ccccc2)C2CCNCC2)cccc1N1CCOCC1]

To a stirred solution of benzyl 4-[(R)-(2-chloro-3-morpholino-phenyl)-phenyl-methyl]piperidine-1-carboxylate (234 mg, 0.459 mmol) in EtOH (4.0 mL) under argon was added palladium on carbon (10% w/w, 48.8 mg, 0.0459 mmol). The reaction mixture was stirred under H₂(g) (0.3 bars) at ambient temperature overnight. The reaction mixture was filtered through a pad of celite and the filtrate was concentrated under reduced pressure to afford 4-[2-chloro-3-[(R)-phenyl(4-piperidyl)methyl]phenyl]morpholine as a colorless oil (143 mg, 93% p., 78% y.). m/z [M+H]⁺=371.3.

### Intermediate 90C: tert-butyl(4S)-5-amino-4-[5-[4-[(R)-(2-chloro-3-morpholino-phenyl)-phenyl-methyl]piperidine-1-carbonyl]-6-methyl-1-oxo-isoindolin-2-yl]-5-oxo-pentanoate

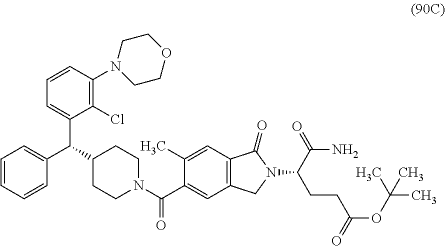 [SMILES: Cc1cc2c(cc1C(=O)N1CCC([C@H](c3ccccc3)c3cccc(N4CCOCC4)c3Cl)CC1)CN([C@@H](CCC(=O)OC(C)(C)C)C(N)=O)C2=O]

In a round-bottom flask, under nitrogen, 2-[(1S)-4-tert-butoxy-1-carbamoyl-4-oxo-butyl]-6-methyl-1-oxo-isoindoline-5-carboxylic acid (135 mg, 0.351 mmol) and 4-[2-chloro-3-[(R)-phenyl(4-piperidyl)methyl]phenyl]morpholine (140 mg, 0.351 mmol) were in suspension in dry DCM (1.4 mL). DIPEA (245 μL, 1.40 mmol) was added, followed by a solution of T3P in DCM (53% w/w, 168 mg, 0.527 mmol) at ambient temperature. The reaction mixture was stirred at ambient temperature for 5 h. The reaction was quenched with the addition of a saturated aqueous solution of NaHCO₃. EtOAc was added. The layers were separated and the aqueous layer was extracted with EtOAc (2×). The combined organic layers were washed with brine, dried using a phase separator cartridge, and concentrated under reduced pressure. The crude was purified by flash chromatography on silica gel using a gradient of EtOAc/EtOH (3/1) in n-heptane from 2% to 80% to afford tert-butyl(4S)-5-amino-4-[5-[4-[(R)-(2-chloro-3-morpholino-phenyl)-phenyl-methyl]piperidine-1-carbonyl]-6-methyl-1-oxo-isoindolin-2-yl]-5-oxo-pentanoate as a white solid (120 mg, 99% p., 46% y.). m/z [M+H]⁺=729.4. [α]_{D}=+38°mL·dm⁻¹·g⁻¹[20° C., Na lamp, 589 nm, C=1.0 mg/mL, EtOH].

### Example 90

To a stirred solution of tert-butyl(4S)-5-amino-4-[5-[4-[(R)-(2-chloro-3-morpholino-phenyl)-phenyl-methyl]piperidine-1-carbonyl]-6-methyl-1-oxo-isoindolin-2-yl]-5-oxo-pentanoate (120 mg, 0.163 mmol) in dry acetonitrile (1.7 mL) in a sealed vial was added 4-methylbenzenesulfonic acid hydrate (186 mg, 0.977 mmol). The reaction mixture was stirred at 90° C. for 3 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure until most of the acetonitrile evaporated. A saturated aqueous solution of NaHCO₃ was added. The resulting precipitate was filtered, washed with water, and dried under reduced pressure at 50° C. overnight to afford (3S)-3-[5-[4-[(R)-(2-chloro-3-morpholino-phenyl)-phenyl-methyl]piperidine-1-carbonyl]-6-methyl-1-oxo-isoindolin-2-yl]piperidine-2,6-dione as a white powder (38.0 mg, 99.0% p., 35% y.). m/z [M+H]⁺=655.4, ¹H NMR (500 MHz, DMSO-d₆): δ (ppm) 10.98 (s, 1H), 7.61 (br d, J=7.3 Hz, 1H), 7.51-7.43 (m, 1H), 7.43-7.35 (m, 2H), 7.35-7.34 (m, 1H), 7.33 (s, 1H), 7.29-7.21 (m, 2H), 7.21-7.10 (m, 1H), 7.06-6.93 (m, 1H), 5.20-5.02 (m, 1H), 4.58-4.47 (m, 1H), 4.47-4.36 (m, 1H), 4.28 (br d, J=11.0 Hz, 2H), 3.71 (dt, J=15.0, 4.4 Hz, 4H), 3.27-3.14 (m, 1H), 3.04-2.95 (m, 1H), 2.94-2.92 (m, 1H), 2.92-2.82 (m, 4H), 2.82-2.70 (m, 1H), 2.66-2.53 (m, 2H), 2.46-2.36 (m, 1H), 2.25 (s, 3H), 1.99 (s, 1H), 1.69-1.21 (m, 2H), 1.21-0.89 (m, 2H), [α]_{D}=+8° mL·dm⁻¹ g⁻¹[20° C., Na lamp, 589 nm, C=1.0 mg/mL, DMSO].

### Example 91

### (3S)-3-[5-[4-[(R)-[2-chloro-3-(isoxazol-3-ylmethoxy)phenyl]-phenyl-methyl]piperidine-1-carbonyl]-6-methyl-1-oxo-isoindolin-2-yl]piperidine-2,6-dione

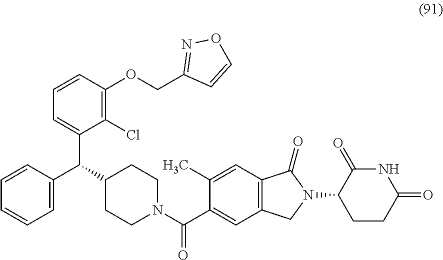 [SMILES: Cc1cc2c(cc1C(=O)N1CCC([C@H](c3ccccc3)c3cccc(OCc4ccon4)c3Cl)CC1)CN([C@H]1CCC(=O)NC1=O)C2=O]

### Intermediate 91A: tert-butyl(4S)-5-amino-4-[5-[4-[(R)-[2-chloro-3-(isoxazol-3-ylmethoxy)phenyl]-phenyl-methyl]piperidine-1-carbonyl]-6-methyl-1-oxo-isoindolin-2-yl]-5-oxo-pentanoate

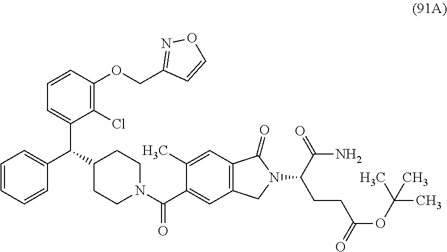 [SMILES: Cc1cc2c(cc1C(=O)N1CCC([C@H](c3ccccc3)c3cccc(OCc4ccon4)c3Cl)CC1)CN([C@@H](CCC(=O)OC(C)(C)C)C(N)=O)C2=O]

In a sealed vial, to a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.211 mmol) in dry THF (1.4 mL) were added successively isoxazol-3-ylmethanol (64.1 μL, 0.634 mmol), triphenylphosphane (166 mg, 0.634 mmol), and then DBAD (150 mg, 0.634 mmol). The reaction mixture was stirred at ambient temperature for 72 h. The reaction mixture was concentrated under reduced pressure. The crude was purified by flash chromatography on silica gel using a gradient of EtOAc/EtOH (3/1) in n-heptane from 5% to 100% to afford tert-butyl(4S)-5-amino-4-[5-[4-[(R)-[2-chloro-3-(isoxazol-3-ylmethoxy)phenyl]-phenyl-methyl]piperidine-1-carbonyl]-6-methyl-1-oxo-isoindolin-2-yl]-5-oxo-pentanoate as a white solid (144 mg, 95% p., 87% y.). m/z [M+H]⁺=741.4. [α]_{D}=+28° mL·dm⁻¹ g⁻¹[20° C., Na lamp, 589 nm, C=1.2 mg/mL, EtOH].

### Example 91

To a stirred solution of tert-butyl(4S)-5-amino-4-[5-[4-[(R)-[2-chloro-3-(isoxazol-3-ylmethoxy)phenyl]-phenyl-methyl]piperidine-1-carbonyl]-6-methyl-1-oxo-isoindolin-2-yl]-5-oxo-pentanoate (140 mg, 0.189 mmol) in acetonitrile (1.3 mL) at ambient temperature was added 4-methylbenzenesulfonic acid hydrate (108 mg, 0.567 mmol). The reaction mixture was stirred at 80° C. for 3 h. The reaction mixture was basified with a saturated aqueous solution of NaHCO₃ and EtOAc was added. The layers were separated, and the aqueous layer was extracted with EtOAc (2×). The combined organic layers were washed with brine, dried over Na₂SO₄, filtered, and concentrated under reduced pressure. The crude was purified by flash chromatography on silica gel using a gradient of EtOAc/EtOH in n-heptane from 10% to 100%. The residue was solubilized in a minimum of acetone and added dropwise to n-pentane. The resulting precipitate was filtered, washed with n-pentane and dried under reduced pressure at 50° C. for 12 h to afford (3S)-3-[5-[4-[(R)-[2-chloro-3-(isoxazol-3-ylmethoxy)phenyl]-phenyl-methyl]piperidine-1-carbonyl]-6-methyl-1-oxo-isoindolin-2-yl]piperidine-2,6-dione as a white powder (52.5 mg, 99.1% p., 41% y.). m/z [M+H]⁺=667.4, ¹H NMR (500 MHz, DMSO-d₆): δ (ppm) 10.98 (s, 1H), 8.95 (dd, J=12.2, 1.7 Hz, 1H), 7.61 (br d, J=4.0 Hz, 1H), 7.51-7.17 (m, 8H), 7.15-7.04 (m, 1H), 6.73-6.63 (m, 1H), 5.32-5.21 (m, 2H), 5.14-5.06 (m, 1H), 4.51 (br s, 1H), 4.46-4.37 (m, 1H), 4.34-4.26 (m, 1H), 4.26-4.18 (m, 1H), 3.28-3.18 (m, 1H), 2.99 (br d, J=12.3 Hz, 1H), 2.90 (br d, J=3.3 Hz, 1H), 2.76 (br d, J=12.5 Hz, 1H), 2.63-2.53 (m, 2H), 2.41-2.31 (m, 2H), 2.25 (s, 2H), 2.03-1.95 (m, 1H), 1.73-0.94 (m, 4H), [α]_{D}=+15° mL·dm⁻¹ g⁻¹[20° C., Na lamp, 589 nm, C=2.3 mg/mL, DMSO].

### Example 92

### (3S)-3-[5-[4-[(R)-[2-chloro-3-(oxazol-2-ylmethoxy)phenyl]-phenyl-methyl]piperidine-1-carbonyl]-6-methyl-1-oxo-isoindolin-2-yl]piperidine-2,6-dione

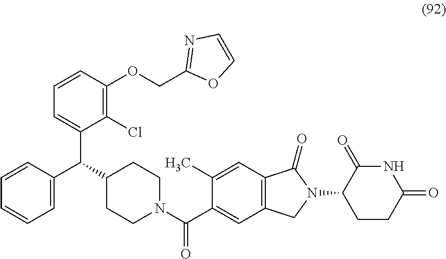 [SMILES: Cc1cc2c(cc1C(=O)N1CCC([C@H](c3ccccc3)c3cccc(OCc4ncco4)c3Cl)CC1)CN([C@H]1CCC(=O)NC1=O)C2=O]

### Intermediate 92A: tert-butyl(4S)-5-amino-4-[5-[4-[(R)-[2-chloro-3-(oxazol-2-ylmethoxy)phenyl]-phenyl-methyl]piperidine-1-carbonyl]-6-methyl-1-oxo-isoindolin-2-yl]-5-oxo-pentanoate

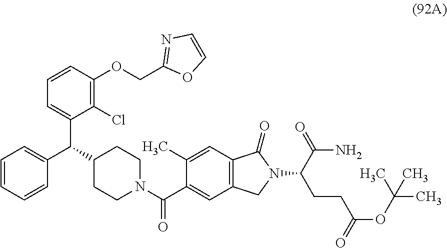 [SMILES: Cc1cc2c(cc1C(=O)N1CCC([C@H](c3ccccc3)c3cccc(OCc4ncco4)c3Cl)CC1)CN([C@@H](CCC(=O)OC(C)(C)C)C(N)=O)C2=O]

In a sealed vial, to a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.211 mmol) in dry THF (1.4 mL) were added successively 1,3-oxazol-2-ylmethanol (65.4 mg, 0.634 mmol), triphenylphosphane (166 mg, 0.634 mmol), and then DBAD (150 mg, 0.634 mmol). The reaction mixture was stirred at ambient temperature for 72 h. The reaction mixture was concentrated under reduced pressure. The crude was purified by flash chromatography on silica gel using a gradient of EtOAc/EtOH (3/1) in n-heptane from 5% to 100% to afford tert-butyl(4S)-5-amino-4-[5-[4-[(R)-[2-chloro-3-(oxazol-2-ylmethoxy)phenyl]-phenyl-methyl]piperidine-1-carbonyl]-6-methyl-1-oxo-isoindolin-2-yl]-5-oxo-pentanoate as a pale yellow foam (145 mg, 95% p. 88% y.). m/z [M+H]⁺=741.4. [α]_{D}=+21.60°·mL·dm⁻¹ g⁻¹[20° C., Na lamp, 589 nm, C=1.25 mg/mL, EtOH].

### Example 92

To a stirred solution of tert-butyl(4S)-5-amino-4-[5-[4-[(R)-[2-chloro-3-(oxazol-2-ylmethoxy)phenyl]-phenyl-methyl]piperidine-1-carbonyl]-6-methyl-1-oxo-isoindolin-2-yl]-5-oxo-pentanoate (149 mg, 0.201 mmol) in acetonitrile (1.3 mL) at ambient temperature was added 4-methylbenzenesulfonic acid hydrate (115 mg, 0.603 mmol). The reaction mixture was stirred at 80° C. for 3 h. The reaction mixture was basified with a saturated aqueous solution of NaHCO₃ and EtOAc was added. The layers were separated, and the aqueous layer was extracted with EtOAc (2×). The combined organic layers were washed with brine, dried over Na₂SO₄, filtered, and concentrated under reduced pressure. The crude was purified by flash chromatography on silica gel using a gradient of EtOAc/EtOH (3/1) in n-heptane from 10% to 100%. The resulting residue was solubilized in a minimum of acetone and added dropwise to n-pentane. The resulting precipitate was filtered, washed with n-pentane, and dried under reduced pressure at 50° C. for 12 h to afford (3S)-3-[5-[4-[(R)-[2-chloro-3-(oxazol-2-ylmethoxy)phenyl]-phenyl-methyl]piperidine-1-carbonyl]-6-methyl-1-oxo-isoindolin-2-yl]piperidine-2,6-dione as a white powder (26.3 mg, 98.6% p., 19% y.). m/z [M+H]⁺=667.4, ¹H NMR (600 MHz, DMSO-d₆): δ (ppm) 11.06-10.84 (m, 1H), 8.19-8.11 (m, 1H), 7.64-7.57 (m, 1H), 7.50-7.02 (m, 10H), 5.33-5.20 (m, 2H), 5.15-4.98 (m, 1H), 4.56-4.14 (m, 4H), 3.26-3.14 (m, 1H), 3.05-2.54 (m, 5H), 2.42-2.19 (m, 4H), 2.04-1.88 (m, 1H), 1.70-0.81 (m, 4H), [α]_{D}=+11° mL·dm⁻¹·g⁻¹[20° C., Na lamp, 589 nm, C=1.5 mg/mL, DMSO].

### Example 93

### (3S)-3-[6-methyl-1-oxo-5-[4-[phenyl(1,2,3,4-tetrahydroquinolin-5-yl)methyl]piperidine-1-carbonyl]isoindolin-2-yl]piperidine-2,6-dione

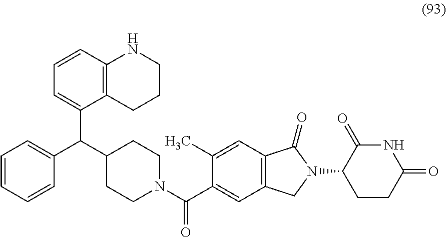 [SMILES: Cc1cc2c(cc1C(=O)N1CCC(C(c3ccccc3)c3cccc4c3CCCN4)CC1)CN([C@H]1CCC(=O)NC1=O)C2=O]

### Intermediate 93A: tert-butyl 5-[(1-tert-butoxycarbonyl-4-piperidylidene)-phenyl-methyl]-3,4-dihydro-2H-quinoline-1-carboxylate

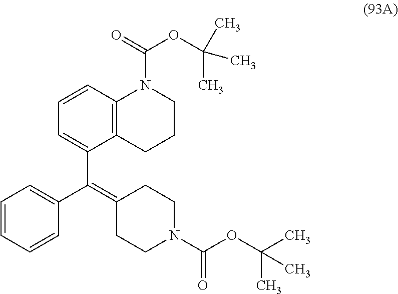 [SMILES: CC(C)(C)OC(=O)N1CCC(=C(c2ccccc2)c2cccc3c2CCCN3C(=O)OC(C)(C)C)CC1]

To a stirred solution of tert-butyl 4-[phenyl-(4,4,5,5-tetramethyl-1,3,2-dioxaborolan-2-yl)methylene]piperidine-1-carboxylate (400 mg, 1.00 mmol) and tert-butyl 5-bromo-1,2,3,4-tetrahydroquinoline-1-carboxylate (375 mg, 1.20 mmol) in CPME (4.0 mL) was added a solution of K₂CO₃ (415 mg, 3.01 mmol) in water (1.0 mL). The reaction mixture was degassed with argon for 5 min then Pd(PPh3) 4 (116 mg, 0.100 mmol) was added. The reaction mixture was stirred at 110° C. overnight. The reaction was quenched with the addition of a saturated aqueous solution of NH₄Cl. EtOAc was added to the mixture. The layers were separated and the aqueous layer was extracted with EtOAc (2×). The combined organic layers were washed with brine, dried using a phase separator, and concentrated under reduced pressure. The crude was purified by flash chromatography on silica gel using a gradient of EtOAc in n-heptane from 0% to 50% to afford tert-butyl(5-[(1-tert-butoxycarbonyl-4-piperidylidene)-phenyl-methyl]-3,4-dihydro-2H-quinoline-1-carboxylate as an orange gum (354 mg, 100% p., 70% y.). m/z [M+Na]⁺=527.3, ¹H NMR (400 MHz, DMSO-d₆): δ (ppm) 7.45 (dd, J=8.3, 1.2 Hz, 1H), 7.31 (dd, J=8.1, 6.8 Hz, 2H), 7.25-7.18 (m, 1H), 7.18-7.07 (m, 3H), 6.88 (dd, J=7.5, 1.2 Hz, 1H), 3.48 (dd, J=13.2, 6.4 Hz, 1H), 3.42-3.32 (m, 2H), 2.62 (dt, J=16.3, 6.4 Hz, 1H), 2.41-2.30 (m, 2H), 2.25 (ddd, J=13.8, 6.9, 4.2 Hz, 1H), 2.07-1.90 (m, 2H), 1.73 (p, J=6.4 Hz, 2H), 1.45 (s, 9H), 1.39 (s, 9H), 1.32-1.12 (m, 3H).

### Intermediate 93B: tert-butyl 5-[(1-tert-butoxycarbonyl-4-piperidyl)-phenyl-methyl]-3,4-dihydro-2H-quinoline-1-carboxylate

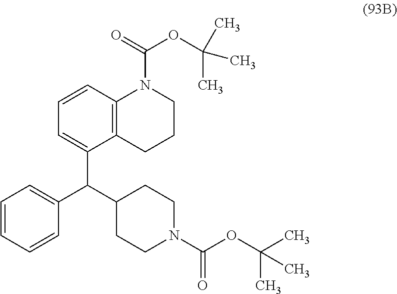 [SMILES: CC(C)(C)OC(=O)N1CCC(C(c2ccccc2)c2cccc3c2CCCN3C(=O)OC(C)(C)C)CC1]

In a sealed vial, a stirred solution of tert-butyl 5-[(1-tert-butoxycarbonyl-4-piperidylidene)-phenyl-methyl]-3,4-dihydro-2H-quinoline-1-carboxylate (350 mg, 0.694 mmol) in EtOH (6.9 mL) was degassed with argon for 5 min and then palladium on carbon (10% w/w, 148 mg, 0.139 mmol) and triethylsilane (554 μL, 3.47 mmol) were added. The mixture was stirred at 70° C. overnight and then cooled to ambient temperature. The suspension was filtered over glass fiber and rinsed with EtOAc. The filtrate was concentrated under reduced pressure to afford tert-butyl 5-[(1-tert-butoxycarbonyl-4-piperidyl)-phenyl-methyl]-3,4-dihydro-2H-quinoline-1-carboxylate as a pale yellow oil (449 mg, 99% p., quant. yield)). m/z [M+Na+]⁺=529.5.

### Intermediate 93C: 5-[phenyl(4-piperidyl)methyl]-1,2,3,4-tetrahydroquinoline dihydrochloride

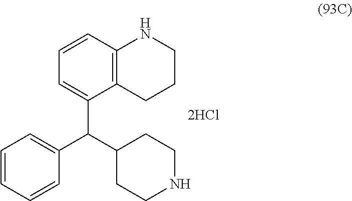 [SMILES: c1ccc(C(c2cccc3c2CCCN3)C2CCNCC2)cc1]

To a stirred solution of tert-butyl 5-[(1-tert-butoxycarbonyl-4-piperidyl)-phenyl-methyl]-3,4-dihydro-2H-quinoline-1-carboxylate (445 mg, 0.878 mmol) in 1,4-dioxane (4.4 mL) was added a 4 M solution of HCl (4.4 mL, 17.6 mmol) dropwise. The reaction mixture was stirred overnight at ambient temperature. Et₂O(88 mL) was added and the reaction mixture was stirred for 1 h. The suspension was filtered, washed with Et₂O, and dried under reduced pressure to afford 5-[phenyl(4-piperidyl)methyl]-1,2,3,4-tetrahydroquinoline dihydrochloride as a white solid (208 mg, 99% p., 62% y.). m/z [M+H]⁺=307.4.

### Intermediate 93D: tert-butyl(4S)-5-amino-4-[6-methyl-1-oxo-5-[4-[phenyl(1,2,3,4-tetrahydroquinolin-5-yl)methyl]piperidine-1-carbonyl]isoindolin-2-yl]-5-oxo-pentanoate

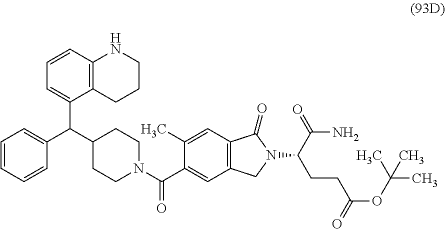 [SMILES: Cc1cc2c(cc1C(=O)N1CCC(C(c3ccccc3)c3cccc4c3CCCN4)CC1)CN([C@@H](CCC(=O)OC(C)(C)C)C(N)=O)C2=O]

To a stirred solution of 5-[phenyl(4-piperidyl)methyl]-1,2,3,4-tetrahydroquinoline dihydrochloride (207 mg, 0.545 mmol) in DCM (5.4 mL) were added successively DIPEA (476 μL, 2.72 mmol), 2-[(1S)-4-tert-butoxy-1-carbamoyl-4-oxo-butyl]-6-methyl-1-oxo-isoindoline-5-carboxylic acid (205 mg, 0.545 mmol), and then a solution of T3P in DCM (53% w/w, 459 μL, 0.817 mmol). The reaction mixture was stirred for 2 h. The reaction was quenched with the addition of water. The aqueous layer was extracted with EtOAc (3×). The combined organic layers were washed with brine, dried over Na₂SO₄, filtered, and concentrated under reduced pressure. The crude was purified by reverse-phase chromatography using a gradient of acetonitrile in water from 10% to 100% (0.1% AcOH). The desired fractions were combined and acetonitrile was removed under reduced pressure. The aqueous layer was basified with a saturated aqueous solution of NaHCO₃. The aqueous layer was extracted with EtOAc (3×). The combined organic layers were washed brine, dried over Na₂SO₄, filtered, and concentrated under reduced pressure to afford tert-butyl(4S)-5-amino-4-[6-methyl-1-oxo-5-[4-[phenyl(1,2,3,4-tetrahydroquinolin-5-yl)methyl]piperidine-1-carbonyl]isoindolin-2-yl]-5-oxo-pentanoate as an off-white solid (195 mg, 98% p., 53% y.). m/z [M+H]⁺=828.4.

### Example 93

To a stirred solution of tert-butyl(4S)-5-amino-4-[6-methyl-1-oxo-5-[4-[(R)-phenyl(1,2,3,4-tetrahydroquinolin-5-yl)methyl]piperidine-1-carbonyl]isoindolin-2-yl]-5-oxo-pentanoate (40.0 mg, 0.0602 mmol) in acetonitrile (600 μL) was added 4-methylbenzenesulfonic acid hydrate (114 mg, 0.602 mmol). The reaction mixture was stirred at 80° C. for 4 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). Acetonitrile was removed under reduced pressure. The aqueous layer was basified with a saturated aqueous solution of NaHCO₃. The precipitate was triturated for 15 min, filtered, washed with water, and dried under reduced pressure at 50° C. overnight to afford (3S)-3-[6-methyl-1-oxo-5-[4-[phenyl(1,2,3,4-tetrahydroquinolin-5-yl)methyl]piperidine-1-carbonyl]isoindolin-2-yl]piperidine-2,6-dione as a white solid (16.2 mg, 97.7% p., 45% y.). m/z [M+H]⁺=591.4, ¹H NMR (600 MHz, DMSO-d₆): δ (ppm) 11.13-10.93 (m, 1H), 7.63-7.55 (m, 1H), 7.45-7.33 (m, 1H), 7.32-7.27 (m, 2H), 7.27-7.17 (m, 2H), 7.16-7.05 (m, 1H), 6.90-6.77 (m, 1H), 6.74-6.65 (m, 1H), 6.33-6.15 (m, 1H), 5.56-5.39 (m, 1H), 5.10 (br dd, J=13.4, 5.1 Hz, 1H), 4.60-4.46 (m, 1H), 4.46-4.21 (m, 2H), 3.74 (s, 1H), 3.27-3.13 (m, 1H), 3.13-3.00 (m, 2H), 2.99-2.91 (m, 1H), 2.91-2.87 (m, 1H), 2.86-2.75 (m, 1H), 2.74-2.63 (m, 1H), 2.63-2.57 (m, 1H), 2.57-2.51 (m, 1H), 2.44-2.20 (m, 5H), 1.98 (s, 1H), 1.87-1.46 (m, 3H), 1.45-1.19 (m, 1H), 1.15-0.79 (m, 2H).

### Example 94

### (3S)-3-[5-[4-[(R)-[2-chloro-3-(2-hydroxyethylamino)phenyl]-phenyl-methyl]piperidine-1-carbonyl]-6-methyl-1-oxo-isoindolin-2-yl]piperidine-2,6-dione

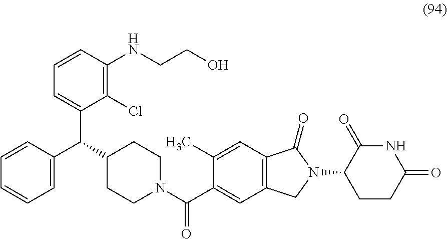 [SMILES: Cc1cc2c(cc1C(=O)N1CCC([C@H](c3ccccc3)c3cccc(NCCO)c3Cl)CC1)CN([C@H]1CCC(=O)NC1=O)C2=O]

### Intermediate 94A: benzyl 4-[(R)-[3-[2-[tert-butyl(dimethyl)silyl]oxyethylamino]-2-chloro-phenyl]-phenyl-methyl]piperidine-1-carboxylate

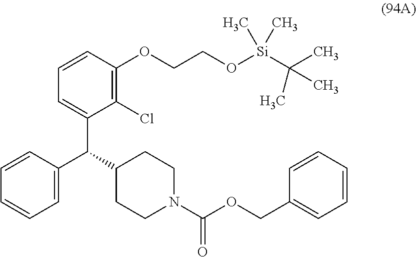 [SMILES: CC(C)(C)[Si](C)(C)OCCOc1cccc([C@@H](c2ccccc2)C2CCN(C(=O)OCc3ccccc3)CC2)c1Cl]

A stirred solution of benzyl 4-[(R)-(3-bromo-2-chloro-phenyl)-phenyl-methyl]piperidine-1-carboxylate (250 mg, 0.476 mmol) and (2-aminoethoxy) (tert-butyl) dimethylsilane (200 μL, 0.952 mmol) in 1,4-dioxane (4.8 mL) at ambient temperature was degassed with argon for 10 min. Next, [1-(2-diphenylphosphanyl-1-naphthyl)-2-naphthyl]-diphenyl-phosphane (61.1 mg, 0.0952 mmol) and Pd(OAc)₂(10.8 mg, 0.0476 mmol) were added. The reaction mixture was stirred for 16 h at 100° C. The reaction mixture was cooled to ambient temperature, poured in a saturated aqueous solution of NaHCO₃, and EtOAc. Layers were separated and the aqueous layer was extracted with EtOAc (2×). The combined organic layers were washed with brine, dried using a phase separator, and concentrated under reduced pressure. The crude was purified by flash chromatography on silica gel using a gradient of EtOAc in cyclohexane from 2% to 20% to afford benzyl 4-[(R)-[3-[2-[tert-butyl(dimethyl)silyl]oxyethylamino]-2-chloro-phenyl]-phenyl-methyl]piperidine-1-carboxylate as a pale yellow solid (195 mg, 97% p., 67% y.). m/z [M+H]⁺=593.4, ¹H NMR (400 MHz, DMSO-d₆): δ (ppm) 7.40-7.22 (m, 10H), 7.19-7.08 (m, 2H), 6.91 (dd, J=7.9, 1.4 Hz, 1H), 6.56 (dd, J=8.2, 1.3 Hz, 1H), 5.09 (t, J=5.8 Hz, 1H), 5.05 (s, 2H), 4.09 (d, J=11.2 Hz, 1H), 3.95 (d, J=13.2 Hz, 2H), 3.73 (td, J=5.8, 2.5 Hz, 2H), 3.19 (p, J=6.1 Hz, 2H), 2.80-2.75 (m, 3H), 1.52 (m, J=13.1 Hz, 1H), 1.36 (m, J=13.1 Hz, 1H), 1.14-0.89 (m, 2H), 0.84 (s, 8H), 0.01 (d, J=3.0 Hz, 6H).

### Intermediate 94B: N-[2-[tert-butyl(dimethyl)silyl]oxyethyl]-2-chloro-3-[(R)-phenyl(4-piperidyl)methyl]aniline

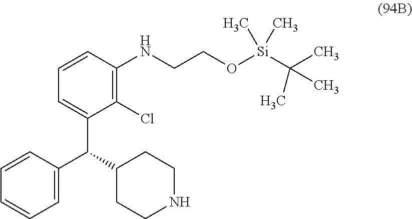 [SMILES: CC(C)(C)[Si](C)(C)OCCNc1cccc([C@@H](c2ccccc2)C2CCNCC2)c1Cl]

To a stirred solution of benzyl 4-[(R)-[3-[2-[tert-butyl(dimethyl)silyl]oxyethylamino]-2-chloro-phenyl]-phenyl-methyl]piperidine-1-carboxylate (195 mg, 0.319 mmol) in EtOH (2.0 mL) under argon was added palladium on carbon (10% w/w, 33.9 mg, 0.0319 mmol). The reaction mixture was stirred under H₂(g) (0.3 bars) at ambient temperature for 48 h. The reaction mixture was filtered through a pad of celite and the filtrate was concentrated under reduced pressure to afford N-[2-[tert-butyl(dimethyl)silyl]oxyethyl]-2-chloro-3-[(R)-phenyl(4-piperidyl)methyl]aniline as a colorless oil (130 mg, 93% p., 83% y.). m/z [M+H]⁺=459.4.

### Intermediate 94C: tert-butyl(4S)-5-amino-4-[5-[4-[(R)-[3-[2-[tert-butyl(dimethyl)silyl]oxyethylamino]-2-chloro-phenyl]-phenyl-methyl]piperidine-1-carbonyl]-6-methyl-1-oxo-isoindolin-2-yl]-5-oxo-pentanoate

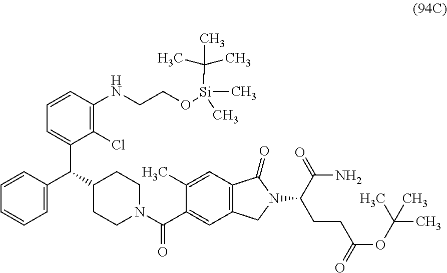 [SMILES: Cc1cc2c(cc1C(=O)N1CCC([C@H](c3ccccc3)c3cccc(NCCO[Si](C)(C)C(C)(C)C)c3Cl)CC1)CN([C@@H](CCC(=O)OC(C)(C)C)C(N)=O)C2=O]

To a stirred suspension of 2-[(1S)-4-tert-butoxy-1-carbamoyl-4-oxo-butyl]-6-methyl-1-oxo-isoindoline-5-carboxylic acid (110 mg, 0.276 mmol) and N-[2-[tert-butyl(dimethyl)silyl]oxyethyl]-2-chloro-3-[(R)-phenyl(4-piperidyl)methyl]aniline (130 mg, 0.263 mmol) in dry DMF (2.3 mL) were added successively DIPEA (184 μL, 1.05 mmol) and HATU (155 mg, 0.395 mmol). The reaction mixture was stirred at ambient temperature for 2 h. The reaction was quenched with the addition of a saturated aqueous solution of NH₄Cl. The aqueous layer was extracted with EtOAc (2×). The combined organic layers were washed with a saturated aqueous solution of NaHCO₃ and brine, dried over Na₂SO₄, filtered, and concentrated under reduced pressure. The crude was purified by flash chromatography on silica gel using a gradient of EtOAc/EtOH (3/1) in n-heptane from 2% to 100%. The desired fractions were combined and concentrated under reduced pressure to afford tert-butyl(4S)-5-amino-4-[5-[4-[(R)-[3-[2-[tert-butyl(dimethyl)silyl]oxyethylamino]-2-chloro-phenyl]-phenyl-methyl]piperidine-1-carbonyl]-6-methyl-1-oxo-isoindolin-2-yl]-5-oxo-pentanoate) as a white foam (108 mg, 95% p., 48% y.). m/z [M+Na]⁺=839.5, ¹H NMR (400 MHz, DMSO-d₆): δ (ppm) 7.95 (s, 1H), 7.55 (d, J=8.1 Hz, 2H), 7.48-7.05 (m, 7H), 6.92 (dd, J=23.0, 7.7 Hz, 1H), 6.63-6.46 (m, 1H), 5.14-5.05 (m, 1H), 4.71 (dd, J=10.2, 3.9 Hz, 1H), 4.58-4.37 (m, 2H), 4.12 (d, J=10.1 Hz, 1H), 3.79-3.67 (m, 2H), 3.28-3.13 (m, 2H), 2.33 (d, J=5.8 Hz, 1H), 2.24 (s, 1H), 2.14 (s, 3H), 1.96 (s, 1H), 1.36-1.19 (m, 9H), 0.91-0.77 (m, 9H), 0.00 (dd, J=12.9, 4.6 Hz, 6H).

### Intermediate 94D: tert-butyl(4S)-5-amino-4-[5-[4-[(R)-[2-chloro-3-(2-hydroxyethylamino)phenyl]-phenyl-methyl]piperidine-1-carbonyl]-6-methyl-1-oxo-isoindolin-2-yl]-5-oxo-pentanoate

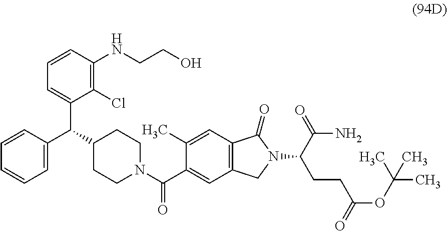 [SMILES: Cc1cc2c(cc1C(=O)N1CCC([C@H](c3ccccc3)c3cccc(NCCO)c3Cl)CC1)CN([C@@H](CCC(=O)OC(C)(C)C)C(N)=O)C2=O]

TBAF (167 mg, 0.251 mmol) supported (1.5 mmol/g) was added to a stirred solution of tert-butyl(4S)-5-amino-4-[5-[4-[(R)-[3-[2-[tert-butyl(dimethyl)silyl]oxyethylamino]-2-chloro-phenyl]-phenyl-methyl]piperidine-1-carbonyl]-6-methyl-1-oxo-isoindolin-2-yl]-5-oxo-pentanoate (108 mg, 0.125 mmol) in dry THF (1.7 mL) at ambient temperature for 16 h. The reaction mixture was filtered and concentrated under pressure to afford tert-butyl(4S)-5-amino-4-[5-[4-[(R)-[2-chloro-3-(2-hydroxyethylamino)phenyl]-phenyl-methyl]piperidine-1-carbonyl]-6-methyl-1-oxo-isoindolin-2-yl]-5-oxo-pentanoate as a white powder (60.0 mg, 96% p., 65% y.). m/z [M+H]⁺=703.4.

### Example 94

To a stirred solution of tert-butyl(4S)-5-amino-4-[5-[4-[(R)-[2-chloro-3-(2-hydroxyethylamino)phenyl]-phenyl-methyl]piperidine-1-carbonyl]-6-methyl-1-oxo-isoindolin-2-yl]-5-oxo-pentanoate (60.0 mg, 0.0819 mmol) in dry acetonitrile (840 μL) was added 4-methylbenzenesulfonic acid hydrate (93.5 mg, 0.491 mmol). The reaction mixture was stirred at 90° C. for 4 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure until evaporation of acetonitrile. A saturated aqueous solution of NaHCO₃ was added. The resulting precipitate was filtered and dried under reduced pressure at 50° C. for 16 h, to afford (3S)-3-[5-[4-[(R)-[2-chloro-3-(2-hydroxyethylamino)phenyl]-phenyl-methyl]piperidine-1-carbonyl]-6-methyl-1-oxo-isoindolin-2-yl]piperidine-2,6-dione as a white powder (7.70 mg, 99.7% p., 15% y.). m/z [M+H]⁺=629.4, ¹H NMR (500 MHz, DMSO-d₆): δ (ppm) 10.98 (s, 1H), 7.63-7.59 (m, 1H), 7.52-7.07 (m, 7H), 7.00-6.84 (m, 1H), 6.59-6.48 (m, 1H), 5.19-5.07 (m, 2H), 4.83-4.75 (m, 1H), 4.61-4.22 (m, 3H), 4.19-4.08 (m, 1H), 3.60-3.52 (m, 2H), 3.26-3.08 (m, 3H), 3.05-2.71 (m, 3H), 2.67-2.52 (m, 2H), 2.47-2.31 (m, 2H), 2.25 (s, 2H), 1.99 (br s, 1H), 1.75-0.84 (m, 4H), [α]_{D}=+16°·mL·dm⁻¹ g⁻¹[20° C., Na lamp, 589 nm, C=1.1 mg/mL, DMSO].

### Example 95

### (3S)-3-[5-[4-[(R)-[3-[(1R)-2-amino-1-methyl-ethoxy]-2-chloro-phenyl]-phenyl-methyl]piperidine-1-carbonyl]-6-methyl-1-oxo-isoindolin-2-yl]piperidine-2,6-dione hydrochloride

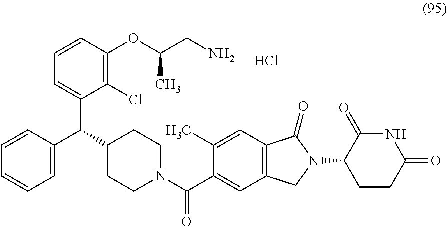 [SMILES: Cc1cc2c(cc1C(=O)N1CCC([C@H](c3ccccc3)c3cccc(O[C@H](C)CN)c3Cl)CC1)CN([C@H]1CCC(=O)NC1=O)C2=O ; Cl]

### Intermediate 95A: tert-butyl(4S)-5-amino-4-[5-[4-[(R)-[3-[(1R)-2-(tert-butoxycarbonylamino)-1-methyl-ethoxy]-2-chloro-phenyl]-phenyl-methyl]piperidine-1-carbonyl]-6-methyl-1-oxo-isoindolin-2-yl]-5-oxo-pentanoate

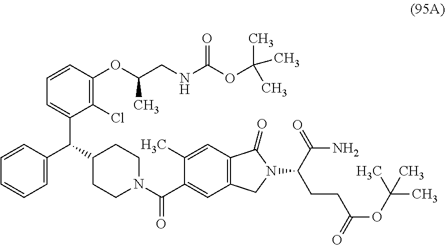 [SMILES: Cc1cc2c(cc1C(=O)N1CCC([C@H](c3ccccc3)c3cccc(O[C@H](C)CNC(=O)OC(C)(C)C)c3Cl)CC1)CN([C@@H](CCC(=O)OC(C)(C)C)C(N)=O)C2=O]

To a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (180 mg, 0.273 mmol) in dry THF (1.8 mL) were added successively triphenylphosphane (215 mg, 0.818 mmol), tert-butyl N-[(2S)-1-hydroxybutan-2-yl]carbamate (158 mg, 0.818 mmol) and then DBAD (194 mg, 0.818 mmol). The reaction mixture was stirred at ambient temperature for 16 h. The reaction mixture was concentrated under reduced pressure. The crude was purified by flash chromatography on silica gel using a gradient of EtOAc/EtOH (3/1) in n-heptane from 5% to 100% to afford tert-butyl(4S)-5-amino-4-[5-[4-[(R)-[3-[(1R)-2-(tert-butoxycarbonylamino)-1-methyl-ethoxy]-2-chloro-phenyl]-phenyl-methyl]piperidine-1-carbonyl]-6-methyl-1-oxo-isoindolin-2-yl]-5-oxo-pentanoate as a white solid (199 mg, 92% p., 82% y.). m/z [M+H]⁺=817.6.

### Example 95

To a stirred solution of tert-butyl(4S)-5-amino-4-[5-[4-[(R)-[3-[(1R)-2-(tert-butoxycarbonylamino)-1-methyl-ethoxy]-2-chloro-phenyl]-phenyl-methyl]piperidine-1-carbonyl]-6-methyl-1-oxo-isoindolin-2-yl]-5-oxo-pentanoate (199 mg, 0.224 mmol) in acetonitrile (2.3 mL) was added 4-methylbenzenesulfonic acid hydrate (213 mg, 1.12 mmol). The reaction mixture was stirred at 80° C. for 5 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure. The solid was solubilized in THF (6.1 mL) and a 2 M solution of HCl in Et₂O(2.0 ml) was added. The reaction mixture was stirred at ambient temperature for 30 min and Et₂O(61 mL) was added. After 1 h, the precipitate was filtered, washed with Et₂O, and dried under reduced pressure overnight to afford (3S)-3-[5-[4-[(R)-[3-[(1R)-2-amino-1-methyl-ethoxy]-2-chloro-phenyl]-phenyl-methyl]piperidine-1-carbonyl]-6-methyl-1-oxo-isoindolin-2-yl]piperidine-2,6-dione hydrochloride as a white powder (49.0 mg, 98.7% p., 32% y.). m/z [M+H]⁺=643.5, ¹H NMR (600 MHz, DMSO-d₆): δ (ppm) 10.97 (br s, 1H), 8.01-7.64 (m, 3H), 7.63-7.56 (m, 1H), 7.50-6.86 (m, 9H), 5.16-5.04 (m, 1H), 4.71-4.12 (m, 5H), 3.28-3.16 (m, 1H), 3.15-2.83 (m, 4H), 2.83-2.69 (m, 1H), 2.65-2.52 (m, 2H), 2.46-2.29 (m, 2H), 2.25 (br d, J=2.5 Hz, 2H), 2.05-1.92 (m, 1H), 1.72-0.67 (m, 7H), [α] D=+13°·mL·dm⁻¹·g⁻¹[20° C., Na lamp, 589 nm, C=1.0 mg/mL, DMSO].

### Example 96

### (3S)-3-[5-[4-[3,4-dihydro-2H-1,4-benzoxazin-8-yl(phenyl)methyl]piperidine-1-carbonyl]-6-methyl-1-oxo-isoindolin-2-yl]piperidine-2,6-dione

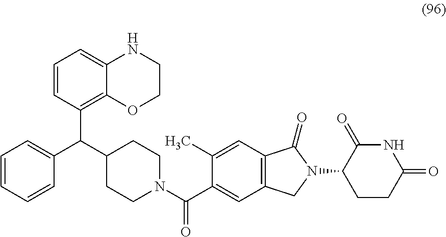 [SMILES: Cc1cc2c(cc1C(=O)N1CCC(C(c3ccccc3)c3cccc4c3OCCN4)CC1)CN([C@H]1CCC(=O)NC1=O)C2=O]

### Intermediate 96A: tert-butyl 8-(4,4,5,5-tetramethyl-1,3,2-dioxaborolan-2-yl)-2,3-dihydro-1,4-benzoxazine-4-carboxylate

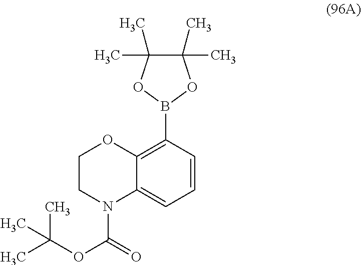 [SMILES: CC(C)(C)OC(=O)N1CCOc2c(B3OC(C)(C)C(C)(C)O3)cccc21]

To a stirred solution of tert-butyl 8-bromo-3,4-dihydro-2H-1,4-benzoxazine-4-carboxylate (500 mg, 1.56 mmol) in CPME (7.8 mL) at ambient temperature was added B₂pin₂(445 mg, 1.72 mmol) and KOAc (464 mg, 4.68 mmol). The reaction mixture was degassed with argon for 15 min and Pd(dppf)Cl₂(128 mg, 0.156 mmol) was added. The reaction mixture was stirred at 100° C. for 2 h. The reaction mixture was filtered through a glass fiber filter and washed with EtOAc. A saturated aqueous solution of NH₄Cl was added to the filtrate. The layers were separated, and the aqueous layer was extracted with EtOAc (2×). The combined organic layers were washed with brine, dried using hydrophobic paper and concentrated under reduced pressure. The crude was purified by flash chromatography on silica gel using a gradient of EtOAc in n-heptane from 0% to 25% to afford expected product as a yellow sticky solid (tert-butyl(8-(4,4,5,5-tetramethyl-1,3,2-dioxaborolan-2-yl)-2,3-dihydro-1,4-benzoxazine-4-carboxylate (304 mg, 56% p., 30% y.). m/z [M+H]⁺=no ionization, 1H NMR (400 MHz, DMSO-d₆): δ (ppm) 7.81-7.74 (m, 1H), 7.28-7.20 (m, 1H), 6.84 (dd, J=8.2, 7.3 Hz, 1H), 4.27-4.16 (m, 2H), 3.80-3.72 (m, 2H), 1.47 (s, 9H), 1.26 (s, 12H).

### Intermediate 96B: tert-butyl 8-[(1-tert-butoxycarbonyl-4-piperidylidene)-phenyl-methyl]-2,3-dihydro-1,4-benzoxazine-4-carboxylate

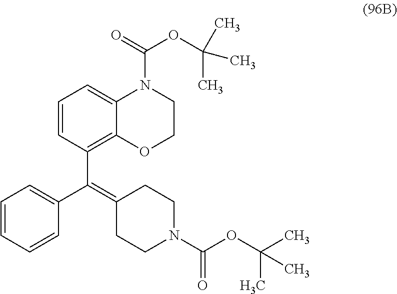 [SMILES: CC(C)(C)OC(=O)N1CCC(=C(c2ccccc2)c2cccc3c2OCCN3C(=O)OC(C)(C)C)CC1]

To a stirred solution of tert-butyl 4-[bromo (phenyl)methylene]piperidine-1-carboxylate (576 mg, 1.64 mmol) and K₂CO₃ (679 mg, 4.91 mmol) in CPME (3.4 mL) and water (840 μL) was added tert-butyl 8-(4,4,5,5-tetramethyl-1,3,2-dioxaborolan-2-yl)-2,3-dihydro-1,4-benzoxazine-4-carboxylate (304 mg, 0.842 mmol). The reaction vessel was purged with nitrogen and Pd(PPh3) 4 (97.2 mg, 0.0842 mmol) was added. The reaction mixture was stirred at 110° C. for 1.5 h. The reaction was quenched with the addition of a saturated aqueous solution of NH₄Cl. EtOAc was added. The layers were separated and the aqueous layer was extracted with EtOAc (2×). The combined organic layers were washed with brine, dried using hydrophobic paper, and concentrated under reduced pressure. The crude was purified by flash chromatography on silica gel using a gradient of EtOAc in n-heptane from 0% to 30% to afford tert-butyl 8-[(1-tert-butoxycarbonyl-4-piperidylidene)-phenyl-methyl]-2,3-dihydro-1,4-benzoxazine-4-carboxylate as a white gum (350 mg, 88% p., 72% y.). m/z [M-Boc-tBu+2H]⁺=351.4, ¹H NMR (400 MHz, DMSO-d₆): δ (ppm) 7.66 (d, J=8.2 Hz, 1H), 7.34-7.23 (m, 2H), 7.23-7.10 (m, 3H), 6.81 (dd, J=8.2, 7.5 Hz, 1H), 6.74 (dd, J=7.5, 1.7 Hz, 1H), 4.26-4.16 (m, 1H), 4.14-4.04 (m, 1H), 3.82-3.70 (m, 2H), 3.35 (d, J=6.3 Hz, 4H), 2.21 (t, J=5.8 Hz, 2H), 2.06-1.97 (m, 2H), 1.48 (s, 9H), 1.39 (s, 9H).

### Intermediate 96C: tert-butyl 8-[(1-tert-butoxycarbonyl-4-piperidyl)-phenyl-methyl]-2,3-dihydro-1,4-benzoxazine-4-carboxylate

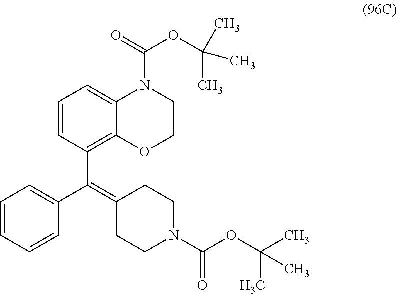 [SMILES: CC(C)(C)OC(=O)N1CCC(=C(c2ccccc2)c2cccc3c2OCCN3C(=O)OC(C)(C)C)CC1]

In a sealed vial, a stirred solution of tert-butyl 8-[(1-tert-butoxycarbonyl-4-piperidylidene)-phenyl-methyl]-2,3-dihydro-1,4-benzoxazine-4-carboxylate (350 mg, 0.690 mmol) in EtOH (6.9 mL) was degassed with argon for 5 min. Palladium on carbon (10% w/w, 147 mg, 0.138 mmol) and triethylsilane (551 μL, 3.45 mmol) were added. The mixture was stirred at 70° C. for 2 h. The suspension was filtered through a glass fiber filter and washed with EtOAc. The filtrate was basified with a saturated aqueous solution of NaHCO₃ and EtOAc was added. The layers were separated and the aqueous layer was extracted with EtOAc (2×). The combined organic layers were washed with brine, dried using a phase separator cartridge, and concentrated under reduced pressure to afford tert-butyl 8-[(1-tert-butoxycarbonyl-4-piperidyl)-phenyl-methyl]-2,3-dihydro-1,4-benzoxazine-4-carboxylate as a colorless oil (650 mg, 52% p., 96% y.). m/z [M+Na]⁺=531.5. ¹H NMR (400 MHz, DMSO-d₆): δ (ppm) 7.53 (d, J=8.3 Hz, 1H), 7.35-7.28 (m, 2H), 7.24 (dd, J=8.3, 6.9 Hz, 2H), 7.18-7.09 (m, 2H), 6.83 (t, J=8.0 Hz, 1H), 4.25 (dt, J=11.0, 4.6 Hz, 1H), 4.17 (dt, J=11.0, 4.5 Hz, 1H), 3.98 (d, J=11.4 Hz, 1H), 3.86 (d, J=12.3 Hz, 2H), 3.72 (t, J=4.7 Hz, 2H), 3.63 (q, J=7.0 Hz, 1H), 2.67 (s, 2H), 2.37 (q, J=11.1 Hz, 1H), 1.55-1.47 (m, 1H), 1.46 (s, 9H), 1.36 (s, 9H), 1.35-1.28 (m, 1H), 1.11 (t, J=7.0 Hz, 1H).

### Intermediate 96D: 8-[phenyl(4-piperidyl)methyl]-3,4-dihydro-2H-1,4-benzoxazine dihydrochloride

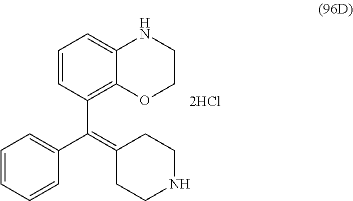 [SMILES: c1ccc(C(=C2CCNCC2)c2cccc3c2OCCN3)cc1]

To a stirred solution of tert-butyl 8-[(1-tert-butoxycarbonyl-4-piperidyl)-phenyl-methyl]-2,3-dihydro-1,4-benzoxazine-4-carboxylate (650 mg, 0.665 mmol) in CPME (3.3 mL) was added a 3 M solution of HCl in CPME (4.4 mL, 13.3 mmol) dropwise. The reaction mixture was stirred overnight. Et₂O(66 mL) was added and the reaction mixture was stirred for 1 h. The suspension was filtered, washed with Et₂O, and dried under reduced pressure to afford 8-[phenyl(4-piperidyl)methyl]-3,4-dihydro-2H-1,4-benzoxazine dihydrochloride as a white solid (154 mg, 98% p., 60% y.). m/z [M+H]⁺=309.3.

### Intermediate 96E: tert-butyl(4S)-5-amino-4-[5-[4-[3,4-dihydro-2H-1,4-benzoxazin-8-yl(phenyl)methyl]piperidine-1-carbonyl]-6-methyl-1-oxo-isoindolin-2-yl]-5-oxo-pentanoate

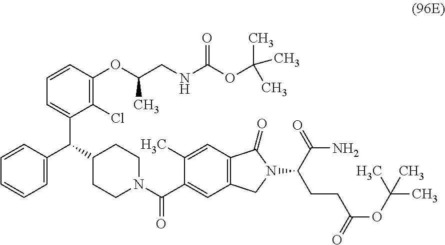 [SMILES: Cc1cc2c(cc1C(=O)N1CCC([C@H](c3ccccc3)c3cccc(O[C@H](C)CNC(=O)OC(C)(C)C)c3Cl)CC1)CN([C@@H](CCC(=O)OC(C)(C)C)C(N)=O)C2=O]

To a stirred suspension of 2-[(1S)-4-tert-butoxy-1-carbamoyl-4-oxo-butyl]-6-methyl-1-oxo-isoindoline-5-carboxylic acid (165 mg, 0.416 mmol) and 8-[phenyl(4-piperidyl)methyl]-3,4-dihydro-2H-1,4-benzoxazine dihydrochloride (154 mg, 0.396 mmol) in dry DMF (3.5 mL) were added successively DIPEA (276 μL, 1.58 mmol) and HATU (233 mg, 0.594 mmol). The reaction mixture was stirred at ambient temperature for 2 h. The reaction was quenched with the addition of a saturated aqueous solution of NH₄C1. The aqueous layer was extracted with EtOAc (2×). The combined organic layers were washed with a saturated aqueous solution of NaHCO₃ and brine, dried over Na₂SO₄, filtered, and concentrated under reduced pressure. The crude was purified by flash chromatography on silica gel using a gradient of EtOAc/EtOH (3/1) in n-heptane from 2% to 100%. The desired fractions were concentrated under reduced pressure to afford tert-butyl(4S)-5-amino-4-[5-[4-[3,4-dihydro-2H-1,4-benzoxazin-8-yl(phenyl)methyl]piperidine-1-carbonyl]-6-methyl-1-oxo-isoindolin-2-yl]-5-oxo-pentanoate as a white powder (163 mg, 100% p, 62% y.). m/z [M+H]⁺=667.5.

### Example 96

To a stirred solution of tert-butyl(4S)-5-amino-4-[5-[4-[3,4-dihydro-2H-1,4-benzoxazin-8-yl(phenyl)methyl]piperidine-1-carbonyl]-6-methyl-1-oxo-isoindolin-2-yl]-5-oxo-pentanoate (50.0 mg, 0.0750 mmol) in dry acetonitrile (770 μL) was added 4-methylbenzenesulfonic acid hydrate (85.6 mg, 0.450 mmol). The reaction mixture was stirred at 90° C. for 2 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure until evaporation of acetonitrile. A saturated aqueous solution of NaHCO₃ was added. The resulting precipitate was filtered and dried under reduced pressure at 50° C. overnight to afford (3S)-3-[5-[4-[3,4-dihydro-2H-1,4-benzoxazin-8-yl(phenyl)methyl]piperidine-1-carbonyl]-6-methyl-1-oxo-isoindolin-2-yl]piperidine-2,6-dione as a white powder (18.6 mg, 98.1% p., 41% y.). m/z [M+H]⁺=593.3, ¹H NMR (500 MHz, DMSO-d₆): δ (ppm) 11.12-10.79 (m, 1H), 7.60 (br d, J=5.3 Hz, 1H), 7.49-7.19 (m, 5H), 7.18-7.05 (m, 1H), 6.70-6.52 (m, 2H), 6.43-6.25 (m, 1H), 5.73-5.57 (m, 1H), 5.20-4.96 (m, 1H), 4.59-4.20 (m, 3H), 4.17-3.93 (m, 3H), 3.29-2.55 (m, 7H), 2.48-2.19 (m, 5H), 1.99 (s, 1H), 1.76-1.23 (m, 2H), 1.14-0.80 (m, 2H).

### Example 97

### (3S)-3-[5-[4-[indolin-6-yl(phenyl)methyl]piperidine-1-carbonyl]-6-methyl-1-oxo-isoindolin-2-yl]piperidine-2,6-dione

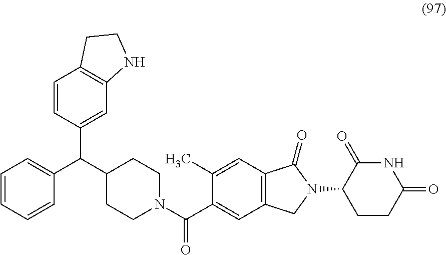 [SMILES: Cc1cc2c(cc1C(=O)N1CCC(C(c3ccccc3)c3ccc4c(c3)NCC4)CC1)CN([C@H]1CCC(=O)NC1=O)C2=O]

### Intermediate 97A: tert-butyl 6-[(1-tert-butoxycarbonyl-4-piperidylidene)-phenyl-methyl]indoline-1-carboxylate

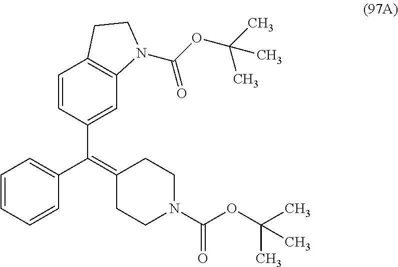 [SMILES: CC(C)(C)OC(=O)N1CCC(=C(c2ccccc2)c2ccc3c(c2)N(C(=O)OC(C)(C)C)CC3)CC1]

In a sealed vial, were combined tert-butyl 6-bromo-2,3-dihydro-1H-indole-1-carboxylate (267 mg, 0.852 mmol), XPhos-Pd-G2 (6.7 mg, 0.00852 mmol), dicyclohexyl-[2-(2,4,6-triisopropylphenyl)phenyl]phosphane (8.1 mg, 0.0170 mmol), hypoboric acid (236 mg, 2.55 mmol), and KOAc (253 mg, 2.55 mmol). The vial was flushed (3×) with argon. EtOH (5.0 mL) was added and the mixture was flushed (3×) with argon. The reaction mixture was stirred at 80° C. for 2 h. Then, a solution of K₂CO₃ (353 mg, 2.55 mmol) in water (1.5 mL) and a solution of tert-butyl 4-[bromo (phenyl)methylene]piperidine-1-carboxylate (300 mg, 0.852 mmol) in EtOH (1.0 mL) previously flushed with argon were added successively to the reaction mixture. The mixture was stirred at 80° C. overnight. The reaction was quenched with the addition of a saturated aqueous solution of NH₄Cl. EtOAc was added. The layers were separated, and the aqueous layer was extracted with EtOAc (2×). The combined organic layers were washed with brine, dried using a phase separator cartridge, and concentrated under reduced pressure. The crude was purified by flash chromatography on silica gel using a gradient of EtOAc in n-heptane from 0% to 100%. The desired fractions were combined, concentrated, and dried under reduced pressure to afford tert-butyl 6-[(1-tert-butoxycarbonyl-4-piperidylidene)-phenyl-methyl]indoline-1-carboxylate as a yellow foam (182 mg, 99% p., 43% y.). m/z [M+H]⁺=491.0, ¹H NMR (400 MHz, DMSO-d₆): δ (ppm) 7.31 (t, J=7.5 Hz, 3H), 7.26-7.18 (m, 1H), 7.11 (dd, J=17.8, 7.5 Hz, 3H), 6.72 (d, J=7.5 Hz, 1H), 3.88 (t, J=8.6 Hz, 2H), 3.36 (d, J=6.7 Hz, 4H), 3.02 (t, J=8.6 Hz, 2H), 2.21 (t, J=6.1 Hz, 4H), 1.41 (d, J=10.3 Hz, 18H).

### Intermediate 97B: tert-butyl 6-[(1-tert-butoxycarbonyl-4-piperidyl)-phenyl-methyl]indoline-1-carboxylate

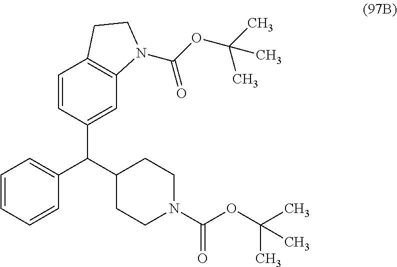 [SMILES: CC(C)(C)OC(=O)N1CCC(C(c2ccccc2)c2ccc3c(c2)N(C(=O)OC(C)(C)C)CC3)CC1]

In a vial with a sealed cap, to a stirred suspension of tert-butyl 6-[(1-tert-butoxycarbonyl-4-piperidylidene)-phenyl-methyl]indoline-1-carboxylate (180 mg, 0.363 mmol) in EtOH (3.6 mL) and palladium on carbon (10% w/w, 77.3 mg, 0.0726 mmol) under argon were added triethylsilane (235 μL, 1.45 mmol). The suspension was stirred at 75° C. for 3 h. The suspension was filtered through a glass fiber filter and rinsed with EtOH. The filtrate was concentrated under reduced pressure and dried under reduced pressure to afford tert-butyl 6-[(1-tert-butoxycarbonyl-4-piperidyl)-phenyl-methyl]indoline-1-carboxylate as a colorless oil (333 mg, 58% p., quant. yield)). m/z [M+Na]⁺=515.2, ¹H NMR (400 MHz, DMSO-d₆): δ (ppm) 7.28 (dt, J=15.1, 7.7 Hz, 4H), 7.14 (t, J=7.1 Hz, 1H), 7.08 (d, J=7.6 Hz, 1H), 6.94 (d, J=7.7 Hz, 1H), 3.85 (t, J=8.6 Hz, 4H), 3.54 (d, J=11.1 Hz, 1H), 2.96 (t, J=8.6 Hz, 2H), 2.67 (s, 2H), 2.32 (s, 1H), 1.43 (d, J=56.4 Hz, 21H), 1.04-0.92 (m, 2H).

### Intermediate 97C: 6-[phenyl(4-piperidyl)methyl]indoline dihydrochloride

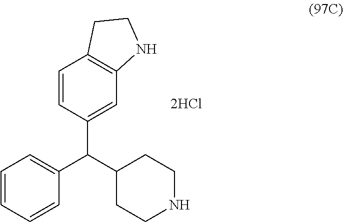 [SMILES: c1ccc(C(c2ccc3c(c2)NCC3)C2CCNCC2)cc1]

In a round-bottom flask, tert-butyl 6-[(1-tert-butoxycarbonyl-4-piperidyl)-phenyl-methyl]indoline-1-carboxylate (333 mg, 0.372 mmol) was treated with a 3 M solution of HCl in CPME (2.5 mL, 7.44 mmol). The reaction mixture was stirred at ambient temperature for 6 h. The suspension was filtered, washed with Et₂O, and dried under reduced pressure to afford 6-[phenyl(4-piperidyl)methyl]indoline dihydrochloride as a white solid (99.1 mg, 77% p., 56% y.). m/z [M+H]⁺=293.3, ¹H NMR (400 MHz, DMSO-d₆): δ (ppm) 8.65 (s, 2H), 7.40-7.33 (m, 2H), 7.29 (t, J=7.6 Hz, 2H), 7.24-6.96 (m, 4H), 3.71-3.50 (m, 3H), 3.23 (d, J=12.9 Hz, 3H), 3.00 (t, J=7.9 Hz, 2H), 2.82 (t, J=11.0 Hz, 2H), 2.52 (s, 1H), 1.56 (t, J=15.3 Hz, 3H), 1.25 (s, 2H).

### Intermediate 97D: tert-butyl(4S)-5-amino-4-[5-[4-[indolin-6-yl(phenyl)methyl]piperidine-1-carbonyl]-6-methyl-1-oxo-isoindolin-2-yl]-5-oxo-pentanoate

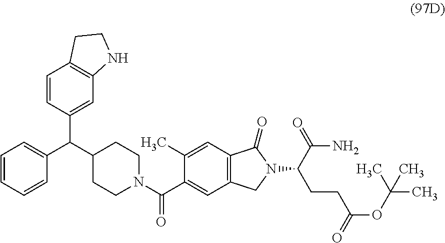 [SMILES: Cc1cc2c(cc1C(=O)N1CCC(C(c3ccccc3)c3ccc4c(c3)NCC4)CC1)CN([C@@H](CCC(=O)OC(C)(C)C)C(N)=O)C2=O]

In a round-bottom flask, were combined 6-methyl-1-oxo-2-[(1S)-4-tert-butoxy-1-carbamoyl-4-oxo-butyl]isoindoline-5-carboxylic acid (103 mg, 0.268 mmol) and 6-[phenyl(4-piperidyl)methyl]indoline dihydrochloride (98.0 mg, 0.268 mmol) in dry DCM (1.1 mL). Then, DIPEA (234 μL, 1.34 mmol) was added, followed by a solution of T3P 53% in DCM (226 μL, 0.402 mmol) at 0-5° C. (ice bath). The reaction mixture was allowed to warm up to ambient temperature for 30 min. The reaction was quenched with the addition of a saturated aqueous solution of NH₄C1. DCM was added. The layers were separated, and the aqueous layer was extracted with DCM. The combined organic layers were washed with brine, dried over Na₂SO₄, filtered, and concentrated under reduced pressure. The crude was purified by flash chromatography on silica gel using a gradient of EtOAc/EtOH (3/1) in n-heptane from 5% to 100% to afford tert-butyl(4S)-5-amino-4-[5-[4-[indolin-6-yl(phenyl)methyl]piperidine-1-carbonyl]-6-methyl-1-oxo-isoindolin-2-yl]-5-oxo-pentanoate as a colorless oil (76.0 mg, 100% p., 44% y.). m/z [M+H]⁺=651.6.

### Example 97

To a stirred solution of tert-butyl(4S)-5-amino-4-[5-[4-[indolin-6-yl(phenyl)methyl]piperidine-1-carbonyl]-6-methyl-1-oxo-isoindolin-2-yl]-5-oxo-pentanoate (70.0 mg, 0.108 mmol) in dry acetonitrile (1.1 mL) was added 4-methylbenzenesulfonic acid hydrate (123 mg, 0.645 mmol). The reaction mixture was stirred at 80° C. for 2 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure until evaporation of acetonitrile. A saturated aqueous solution of NaHCO₃ was added. The resulting precipitate was filtered and dried under reduced pressure at 50° C. overnight to afford (3S)-3-[5-[4-[indolin-6-yl(phenyl)methyl]piperidine-1-carbonyl]-6-methyl-1-oxo-isoindolin-2-yl]piperidine-2,6-dione as a white powder (16.5 mg, 94.7% p., 25% y.). m/z [M+H]⁺=577.4, ¹H NMR (600 MHz, DMSO-d₆): δ (ppm) 10.97 (br s, 1H), 7.60 (br s, 1H), 7.50-7.02 (m, 6H), 6.95-6.80 (m, 1H), 6.59-6.35 (m, 2H), 5.48-5.26 (m, 1H), 5.20-4.97 (m, 1H), 4.61-4.18 (m, 3H), 3.54-3.09 (m, 4H), 3.07-2.68 (m, 5H), 2.66-2.54 (m, 1H), 2.46-2.14 (m, 5H), 2.07-1.86 (m, 1H), 1.74-0.74 (m, 4H).

### Example 98

### (3S)-3-[5-[4-[(R)-(2-chloro-3-ethoxy-phenyl)-phenyl-methyl]piperidine-1-carbonyl]-6-methyl-1-oxo-isoindolin-2-yl]piperidine-2,6-dione

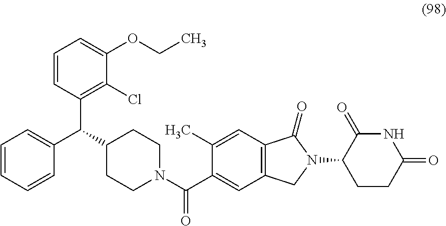 [SMILES: CCOc1cccc([C@@H](c2ccccc2)C2CCN(C(=O)c3cc4c(cc3C)C(=O)N([C@H]3CCC(=O)NC3=O)C4)CC2)c1Cl]

### Intermediate 98A: tert-butyl(4S)-5-amino-4-[5-[4-[(R)-(2-chloro-3-ethoxy-phenyl)-phenyl-methyl]piperidine-1-carbonyl]-6-methyl-1-oxo-isoindolin-2-yl]-5-oxo-pentanoate

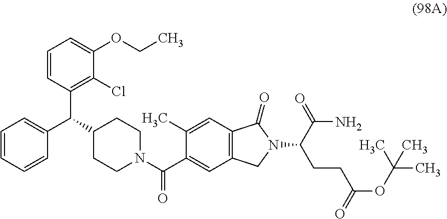 [SMILES: CCOc1cccc([C@@H](c2ccccc2)C2CCN(C(=O)c3cc4c(cc3C)C(=O)N([C@@H](CCC(=O)OC(C)(C)C)C(N)=O)C4)CC2)c1Cl]

To a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.227 mmol) in dry THF (1.5 mL) were added successively triphenylphosphane (179 mg, 0.682 mmol), EtOH (39.7 μL, 0.682 mmol), and then DBAD (162 mg, 0.682 mmol). The reaction mixture was stirred at ambient temperature overnight. The reaction mixture was concentrated under reduced pressure. The crude was purified by flash chromatography on silica gel using a gradient of EtOAc/EtOH (3/1) in n-heptane from 5% to 100% to afford tert-butyl(4S)-5-amino-4-[5-[4-[(R)-(2-chloro-3-ethoxy-phenyl)-phenyl-methyl]piperidine-1-carbonyl]-6-methyl-1-oxo-isoindolin-2-yl]-5-oxo-pentanoate as a white solid (128 mg, 96% p., 79% y.). m/z [M+H]⁺=688.5.

### Example 98

To a stirred solution of tert-butyl(4S)-5-amino-4-[5-[4-[(R)-(2-chloro-3-ethoxy-phenyl)-phenyl-methyl]piperidine-1-carbonyl]-6-methyl-1-oxo-isoindolin-2-yl]-5-oxo-pentanoate (128 mg, 0.179 mmol) in acetonitrile (1.8 mL) was added 4-methylbenzenesulfonic acid hydrate (102 mg, 0.536 mmol). The reaction mixture was stirred at 80° C. for 5 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure until acetonitrile evaporated. The resulting aqueous suspension was basified with a saturated aqueous solution of NaHCO₃. The resulting suspension was filtered and dried under reduced pressure at 50° C. for 48 hours to afford (3S)-3-[5-[4-[(R)-(2-chloro-3-ethoxy-phenyl)-phenyl-methyl]piperidine-1-carbonyl]-6-methyl-1-oxo-isoindolin-2-yl]piperidine-2,6-dione as a white powder (68.4 mg, 99.8% p., 62% y.). m/z [M+H]⁺=614.3, ¹H NMR (600 MHz, DMSO-d₆): δ (ppm) 10.97 (s, 1H), 7.63-7.56 (m, 1H), 7.50-7.20 (m, 7H), 7.20-7.07 (m, 1H), 7.00-6.83 (m, 1H), 5.16-4.99 (m, 1H), 4.57-3.94 (m, 6H), 3.27-2.52 (m, 6H), 2.45-2.19 (m, 4H), 2.04-1.89 (m, 1H), 1.69-0.89 (m, 7H), [α]_{D}=+8° mL·dm⁻¹ g⁻¹[20° C., Na lamp, 589 nm, C=2.1 mg/mL, DMSO].

### Example 99

### (3S)-3-[5-[4-[(R)-[3-(2-aminoethylamino)-2-chloro-phenyl]-phenyl-methyl]piperidine-1-carbonyl]-6-methyl-1-oxo-isoindolin-2-yl]piperidine-2,6-dione hydrochloride

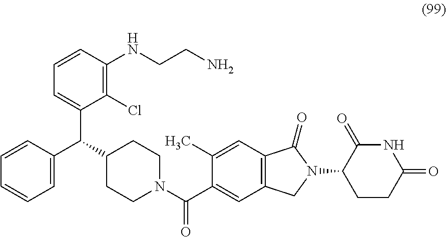 [SMILES: Cc1cc2c(cc1C(=O)N1CCC([C@H](c3ccccc3)c3cccc(NCCN)c3Cl)CC1)CN([C@H]1CCC(=O)NC1=O)C2=O]

### Intermediate 99A: benzyl 4-[(R)-[3-[2-(tert-butoxycarbonylamino)ethylamino]-2-chloro-phenyl]-phenyl-methyl]piperidine-1-carboxylate

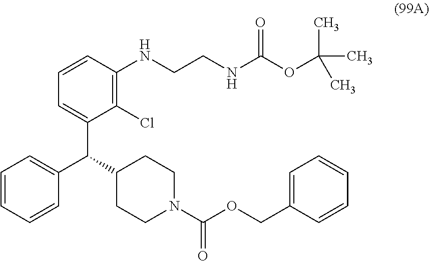 [SMILES: CC(C)(C)OC(=O)NCCNc1cccc([C@@H](c2ccccc2)C2CCN(C(=O)OCc3ccccc3)CC2)c1Cl]

A stirred solution of benzyl 4-[(R)-(3-bromo-2-chloro-phenyl)-phenyl-methyl]piperidine-1-carboxylate (150 mg, 0.292 mmol) and tert-butyl N-(2-aminoethyl) carbamate (96.3 mg, 0.583 mmol) in tert-butanol (5.8 mL) at ambient temperature was degassed with argon for 10 min. Next, t-BuONa (33.6 mg, 0.350 mmol) and Brettphos-Pd-G1 (24.6 mg, 0.0292 mmol) were added. The reaction mixture was stirred at 100° C. overnight. The reaction mixture was cooled to ambient temperature, poured in a saturated aqueous solution of NaHCO₃, and EtOAc was added. The layers were separated, and the aqueous layer was extracted with EtOAc (2×). The combined organic layers were washed with brine, dried using a phase separator cartridge, and concentrated under reduced pressure. The crude was purified by flash chromatography on silica gel using a gradient of EtOAc in cyclohexane from 2% to 50% to afford benzyl 4-[(R)-[3-[2-(tert-butoxycarbonylamino)ethylamino]-2-chloro-phenyl]-phenyl-methyl]piperidine-1-carboxylate as an off-white solid (168 mg, 98% p., 98% y.). m/z [M+H]⁺=578.4, 1H NMR (400 MHz, DMSO-d₆): δ (ppm) 7.43-7.19 (m, 10H), 7.19-7.07 (m, 2H), 7.04-6.94 (m, 1H), 6.89 (d, J=7.6 Hz, 1H), 6.54 (d, J=7.9 Hz, 1H), 5.38 (s, 1H), 5.05 (s, 2H), 4.09 (d, J=11.2 Hz, 1H), 3.95 (d, J=13.2 Hz, 2H), 3.11 (s, 4H), 2.78 (s, 3H), 1.53 (d, J=12.7 Hz, 1H), 1.37 (s, 9H), 0.99 (dq, J=22.7, 10.8 Hz, 2H), [α]_{D}=+58° mL·dm⁻¹ g⁻¹[20° C., Na lamp, 589 nm, C=1.1 mg/mL, EtOH].

### Intermediate 99B: tert-butyl N-[2-[2-chloro-3-[(R)-phenyl(4-piperidyl)methyl]anilino]ethyl]carbamate

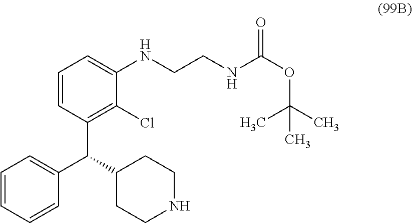 [SMILES: CC(C)(C)OC(=O)NCCNc1cccc([C@@H](c2ccccc2)C2CCNCC2)c1Cl]

To a stirred solution of benzyl 4-[(R)-[3-[2-(tert-butoxycarbonylamino) ethylamino]-2-chloro-phenyl]-phenyl-methyl]piperidine-1-carboxylate (165 mg, 0.280 mmol) in EtOH (2.8 mL) under argon was added palladium on carbon (10% w/w, 33.5 mg, 0.0315 mmol). The reaction mixture was stirred under H₂(g) (0.3-0.5 bar) overnight. The reaction mixture was filtered through a glass fiber filter, and washed with EtOH. The filtrate was concentrated under reduced pressure to afford tert-butyl N-[2-[2-chloro-3-[(R)-phenyl(4-piperidyl)methyl]anilino]ethyl]carbamate as a brown solid (125 mg, 96% p., 97% y.). m/z [M+H]⁺=444.4, ¹H NMR (400 MHz, DMSO-d₆): δ (ppm) 7.36-7.20 (m, 4H), 7.18-7.06 (m, 2H), 6.99 (s, 1H), 6.85 (dd, J=7.9, 1.4 Hz, 1H), 6.52 (d, J=7.8 Hz, 1H), 5.36 (s, 1H), 4.34 (t, J=5.1 Hz, 1H), 4.07 (d, J=11.1 Hz, 1H), 3.44 (qd, J=6.9, 4.5 Hz, 2H), 3.11 (q, J=2.7 Hz, 4H), 2.88-2.79 (m, 2H), 2.39 (dt, J=12.0, 9.6 Hz, 1H), 2.22 (q, J=10.8 Hz, 1H), 1.37 (s, 9H), 1.06-0.85 (m, 3H), [α]_{D}=+74° mL·dm⁻¹·g⁻¹[20° C., Na lamp, 589 nm, C=3.3 mg/mL, EtOH].

### Intermediate 99C: tert-butyl(4S)-5-amino-4-[5-[4-[(R)-[3-[2-(tert-butoxycarbonylamino) ethylamino]-2-chloro-phenyl]-phenyl-methyl]piperidine-1-carbonyl]-6-methyl-1-oxo-isoindolin-2-yl]-5-oxo-pentanoate

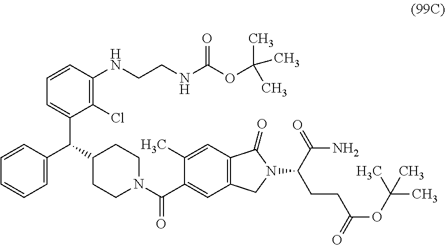 [SMILES: Cc1cc2c(cc1C(=O)N1CCC([C@H](c3ccccc3)c3cccc(NCCNC(=O)OC(C)(C)C)c3Cl)CC1)CN([C@@H](CCC(=O)OC(C)(C)C)C(N)=O)C2=O]

In a round-bottom flask, under nitrogen, 2-[(1S)-4-tert-butoxy-1-carbamoyl-4-oxo-butyl]-6-methyl-1-oxo-isoindoline-5-carboxylic acid (99.7 mg, 0.260 mmol) and tert-butyl N-[2-[2-chloro-3-[(R)-phenyl(4-piperidyl)methyl]anilino]ethyl]carbamate (120 mg, 0.259 mmol) were in suspension in dry DCM (1.1 mL). DIPEA (181 μL, 1.04 mmol) was added, followed by the addition of a solution of T3P in DCM (53% w/w, 0.124 g, 0.389 mmol) at ambient temperature. The reaction mixture was stirred at ambient temperature for 4 h. The reaction was quenched with the addition of a saturated aqueous solution of NH₄Cl. EtOAc was added. The layers were separated, and the aqueous layer was extracted with EtOAc (2×). The combined organic layers were washed with brine, dried using a phase separator cartridge, and concentrated under reduced pressure. The crude was purified by flash chromatography on silica gel using a gradient of EtOAc/EtOH (3/1) in n-heptane from 2% to 100% to afford tert-butyl(4S)-5-amino-4-[5-[4-[(R)-[3-[2-(tert-butoxycarbonylamino)ethylamino]-2-chloro-phenyl]-phenyl-methyl]piperidine-1-carbonyl]-6-methyl-1-oxo-isoindolin-2-yl]-5-oxo-pentanoate as a white solid (105 mg, 95% p., 48% y.). m/z [M+H]⁺=802.6. [α]_{D}=+25° mL·dm⁻¹ g⁻¹[20° C., Na lamp, 589 nm, C=1.1 mg/mL, EtOH].

### Example 99

To a stirred solution of tert-butyl(4S)-5-amino-4-[5-[4-[(R)-[3-[2-(tert-butoxycarbonylamino)ethylamino]-2-chloro-phenyl]-phenyl-methyl]piperidine-1-carbonyl]-6-methyl-1-oxo-isoindolin-2-yl]-5-oxo-pentanoate (105 mg, 0.124 mmol) in acetonitrile (1.0 mL) at ambient temperature was added 4-methylbenzenesulfonic acid hydrate (118 mg, 0.622 mmol). The reaction mixture was stirred at 80° C. for 3 h. The reaction mixture was basified with a saturated aqueous solution of NaHCO₃ and EtOAc was added. The layers were separated and the aqueous layer was extracted with EtOAc (2×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). Fractions were concentrated and basified with a saturated aqueous solution of NaHCO₃. The aqueous layer was extracted with EtOAc (2×). The combined organic layers were washed with brine, dried over Na₂SO₄, filtered, and concentrated under reduced pressure. The residue was solubilized in dry THF (1.0 mL) at ambient temperature and a 2 M solution of HCl in Et₂O(622 μL) was added dropwise. The mixture was stirred at ambient temperature for 10 min and then Et₂O was added. The resulting precipitate was triturated for 30 min, filtered, washed with Et₂O, and dried under reduced pressure at 50° C. for 12 h to afford (3S)-3-[5-[4-[(R)-[3-(2-aminoethylamino)-2-chloro-phenyl]-phenyl-methyl]piperidine-1-carbonyl]-6-methyl-1-oxo-isoindolin-2-yl]piperidine-2,6-dione hydrochloride as a white powder (8.8 mg, 96.9% p., 10% y.). m/z [M+H]⁺=628.5, ¹H NMR (600 MHz, DMSO-d₆): δ (ppm) 10.97 (br s, 1H), 8.07-7.67 (m, 3H), 7.65-6.81 (m, 9H), 6.69-6.50 (m, 1H), 5.63-5.45 (m, 1H), 5.16-4.96 (m, 1H), 4.58-4.03 (m, 4H), 3.32 (s, 3H), 3.07-2.66 (m, 5H), 2.64-2.44 (m, 2H), 2.44-2.15 (m, 4H), 2.06-1.83 (m, 1H), 1.75-0.66 (m, 4H).

### Example 100

### (3S)-3-[5-[4-[(R)-[2-chloro-3-(trideuteriomethoxy)phenyl]-phenyl-methyl]piperidine-1-carbonyl]-6-methyl-1-oxo-isoindolin-2-yl]piperidine-2,6-dione

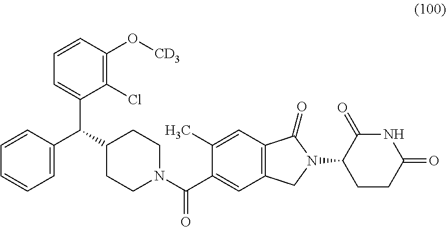 [SMILES: [2H]C([2H])([2H])Oc1cccc([C@@H](c2ccccc2)C2CCN(C(=O)c3cc4c(cc3C)C(=O)N([C@H]3CCC(=O)NC3=O)C4)CC2)c1Cl]

### Intermediate 100A: tert-butyl(4S)-5-amino-4-[5-[4-[(R)-[2-chloro-3-(trideuteriomethoxy) phenyl]-phenyl-methyl]piperidine-1-carbonyl]-6-methyl-1-oxo-isoindolin-2-yl]-5-oxo-pentanoate

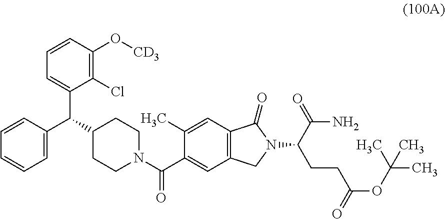 [SMILES: [2H]C([2H])([2H])Oc1cccc([C@@H](c2ccccc2)C2CCN(C(=O)c3cc4c(cc3C)C(=O)N([C@@H](CCC(=O)OC(C)(C)C)C(N)=O)C4)CC2)c1Cl]

To a stirred solution of tert-butyl(4S)-5-amino-4-[5-[4-[(R)-(2-chloro-3-hydroxy-phenyl)-phenyl-methyl]piperidine-1-carbonyl]-6-methyl-1-oxo-isoindolin-2-yl]-5-oxo-pentanoate (150 mg, 0.227 mmol) in CPME (3.0 mL) were added successively triphenylphosphane (179 mg, 0.682 mmol), deuterated methanol (25.3 mg, 0.682 mmol), and then DBAD (162 mg, 0.682 mmol). The reaction mixture was stirred at ambient temperature overnight. Additional deuterated methanol (25.3 mg, 0.682 mmol), triphenylphosphane (179 mg, 0.682 mmol), and DBAD (162 mg, 0.682 mmol) were added and the reaction mixture was stirred at ambient temperature overnight. The reaction mixture was concentrated under reduced pressure. The crude was purified by flash chromatography on silica gel using a gradient of EtOAc/EtOH (3/1) in n-heptane from 5% to 100% to afford tert-butyl(4S)-5-amino-4-[5-[4-[(R)-[2-chloro-3-(trideuteriomethoxy)phenyl]-phenyl-methyl]piperidine-1-carbonyl]-6-methyl-1-oxo-isoindolin-2-yl]-5-oxo-pentanoate as a white solid (124 mg, 97% p., 78% y.). m/z [M+H]⁺=677.4.

### Example 100

To a stirred solution of tert-butyl(4S)-5-amino-4-[5-[4-[(R)-[2-chloro-3-(trideuteriomethoxy)phenyl]-phenyl-methyl]piperidine-1-carbonyl]-6-methyl-1-oxo-isoindolin-2-yl]-5-oxo-pentanoate (123 mg, 0.176 mmol) in acetonitrile (1.8 mL) was added 4-methylbenzenesulfonic acid hydrate (101 mg, 0.529 mmol). The reaction mixture was stirred at 80° C. for 5 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure until acetonitrile evaporated. The resulting aqueous suspension was basified with a saturated aqueous solution of NaHCO₃. The resulting suspension was filtered and dried at 50° C. under reduced pressure for 48 hours to afford (3S)-3-[5-[4-[(R)-[2-chloro-3-(trideuteriomethoxy)phenyl]-phenyl-methyl]piperidine-1-carbonyl]-6-methyl-1-oxo-isoindolin-2-yl]piperidine-2,6-dione as a white powder (68.7 mg, 99.2% p., 64% y.). m/z [M+H]⁺=603.0, ¹H NMR (600 MHz, DMSO-d₆): δ (ppm) 10.97 (br s, 1H), 7.62-7.58 (m, 1H), 7.50-7.20 (m, 7H), 7.20-7.09 (m, 1H), 7.00-6.87 (m, 1H), 5.14-5.02 (m, 1H), 4.57-4.46 (m, 1H), 4.45-4.36 (m, 1H), 4.34-4.16 (m, 2H), 3.29-3.17 (m, 1H), 3.01-2.93 (m, 1H), 2.92-2.84 (m, 1H), 2.81-2.71 (m, 1H), 2.61-2.52 (m, 2H), 2.41-2.30 (m, 2H), 2.24 (s, 2H), 2.02-1.91 (m, 1H), 1.69-0.86 (m, 4H), [α]_{D}=+5° mL·dm⁻¹·g⁻¹[20° C., Na lamp, 589 nm, C=1.7 mg/mL, DMSO].

### Example 101

### (3S)-3-[5-[4-[(R)-[2-chloro-3-(2-fluoroethoxy)phenyl]-phenyl-methyl]piperidine-1-carbonyl]-6-methyl-1-oxo-isoindolin-2-yl]piperidine-2,6-dione

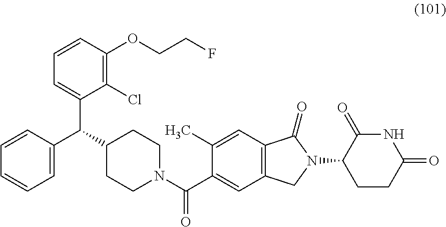 [SMILES: Cc1cc2c(cc1C(=O)N1CCC([C@H](c3ccccc3)c3cccc(OCCF)c3Cl)CC1)CN([C@H]1CCC(=O)NC1=O)C2=O]

### Intermediate 101A: benzyl 4-[(R)-[3-[2-[tert-butyl(dimethyl)silyl]oxyethoxy]-2-chloro-phenyl]-phenyl-methyl]piperidine-1-carboxylate

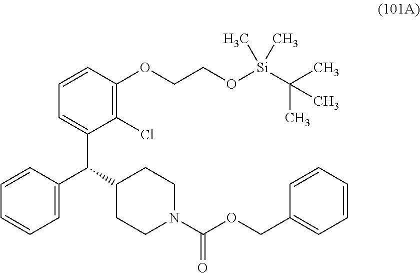 [SMILES: CC(C)(C)[Si](C)(C)OCCOc1cccc([C@@H](c2ccccc2)C2CCN(C(=O)OCc3ccccc3)CC2)c1Cl]

To a stirred solution of benzyl 4-[(R)-(2-chloro-3-hydroxy-phenyl)-phenyl-methyl]piperidine-1-carboxylate (250 mg, 0.568 mmol) in dry THF (3.7 mL) were added successively triphenylphosphane (447 mg, 1.70 mmol), 2-[tert-butyl(dimethyl)silyl]oxyethanol (105 mg, 0.568 mmol), and then DBAD (404 mg, 1.70 mmol). The reaction mixture was stirred at ambient temperature overnight. The reaction mixture was concentrated under reduced pressure. The crude was purified by flash chromatography on silica gel using a gradient of EtOAc in cyclohexane from 1% to 30%. The desired fractions were combined and concentrated under reduced pressure to afford benzyl 4-[(R)-[3-[2-[tert-butyl(dimethyl)silyl]oxyethoxy]-2-chloro-phenyl]-phenyl-methyl]piperidine-1-carboxylate as a colorless oil (165 mg, 98% p., 48% y.). m/z [M+Na]⁺=616.3, ¹H NMR (400 MHz, DMSO-d₆): δ (ppm) 9.35 (s, 1H), 7.40-7.29 (m, 6H), 7.29-7.22 (m, 4H), 7.16 (t, J=7.3 Hz, 1H), 6.99-6.92 (m, 1H), 5.05 (s, 2H), 4.17 (d, J=11.2 Hz, 1H), 4.12-4.00 (m, 2H), 3.99-3.87 (m, 4H), 2.79 (s, 2H), 1.52 (d, J=14.3 Hz, 2H), 1.25 (s, 1H), 1.10-0.88 (m, 2H), 0.84 (s, 9H), 0.04 (d, J=2.3 Hz, 6H).

### Intermediate 101B: benzyl 4-[(R)-[2-chloro-3-(2-hydroxyethoxy)phenyl]-phenyl-methyl]piperidine-1-carboxylate

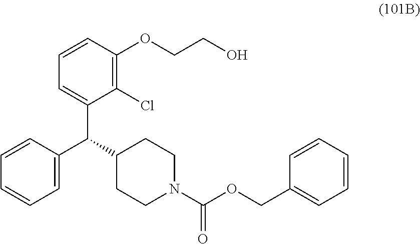 [SMILES: O=C(OCc1ccccc1)N1CCC([C@H](c2ccccc2)c2cccc(OCCO)c2Cl)CC1]

To a stirred solution of benzyl 4-[(R)-[3-[2-[tert-butyl(dimethyl)silyl]oxyethoxy]-2-chloro-phenyl]-phenyl-methyl]piperidine-1-carboxylate (160 mg, 0.256 mmol) in THF (850 μL) under nitrogen was added a 1 M solution TBAF in THF (767 μL, 0.767 mmol). The reaction mixture was stirred at ambient temperature overnight. The mixture was diluted with EtOAc and water. The layers were separated, and the aqueous layer was extracted (2×) with EtOAc. The organic layer was washed with a saturated aqueous solution of NH₄Cl, water and brine, dried over hydrophobic paper, and concentrated under reduced pressure. The crude was purified by flash chromatography on silica gel using a gradient of EtOAc in n-heptane from 5% to 100%. The desired fractions were combined and concentrated to afford benzyl 4-[(R)-[2-chloro-3-(2-hydroxyethoxy)phenyl]-phenyl-methyl]piperidine-1-carboxylate as a colorless oil (65.0 mg, 100% p., 53% y.). m/z [M+H]⁺=480.4.

### Intermediate 101C: benzyl 4-[(R)-[2-chloro-3-(2-fluoroethoxy)phenyl]-phenyl-methyl]piperidine-1-carboxylate

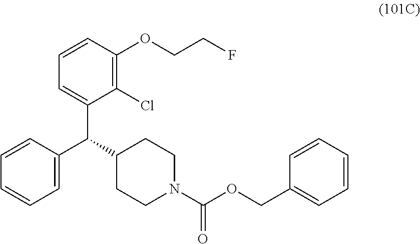 [SMILES: O=C(OCc1ccccc1)N1CCC([C@H](c2ccccc2)c2cccc(OCCF)c2Cl)CC1]

To a stirred solution of benzyl 4-[(R)-[2-chloro-3-(2-hydroxyethoxy)phenyl]-phenyl-methyl]piperidine-1-carboxylate (65.0 mg, 0.135 mmol) in DCM (1.1 mL) at −78° C. under nitrogen was added DAST (24.5 μL, 0.176 mmol). The reaction mixture was stirred at −78° C. for 1 hour. The reaction mixture was stirred at ambient temperature overnight. Additional DAST (37.7 μL, 0.271 mmol) was added and the reaction mixture was stirred for 16 h. The reaction was quenched with the addition of a saturated aqueous solution of NaHCO₃. The mixture was extracted with EtOAc (2×). The combined organic layers were washed brine, dried over Na₂SO₄, filtered, and concentrated under reduced pressure to afford benzyl 4-[(R)-[2-chloro-3-(2-fluoroethoxy)phenyl]-phenyl-methyl]piperidine-1-carboxylate as a yellow oil (57.5 mg, 74% p., 65% y.). m/z [M+H]⁺=482.3, ¹H NMR (400 MHz, DMSO-d₆): δ (ppm) 7.40-7.31 (m, 6H), 7.31-7.23 (m, 4H), 7.16 (t, J=7.3 Hz, 1H), 6.97 (dd, J=5.9, 3.8 Hz, 1H), 5.05 (s, 2H), 4.85-4.76 (m, 1H), 4.67 (t, J=4.0 Hz, 1H), 4.26 (dtd, J=25.5, 8.4, 4.4 Hz, 2H), 4.18 (d, J=11.3 Hz, 1H), 3.96 (d, J=10.1 Hz, 2H), 2.89-2.69 (m, 2H), 1.47 (d, J=12.7 Hz, 1H), 1.37 (s, 1H), 1.24 (s, 1H), 1.22-0.95 (m, 3H).

### Intermediate 101D: 4-[(R)-[2-chloro-3-(2-fluoroethoxy)phenyl]-phenyl-methyl]piperidine hydroiodide

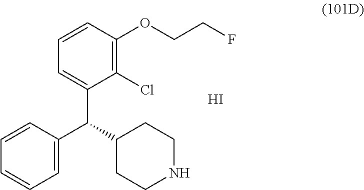

To a stirred solution of benzyl 4-[(R)-[2-chloro-3-(2-fluoroethoxy)phenyl]-phenyl-methyl]piperidine-1-carboxylate (56.0 mg, 0.0929 mmol) in acetonitrile (930 μL) at ambient temperature was added iodo (trimethyl) silane (39.7 μL, 0.279 mmol) dropwise. The reaction mixture was stirred at ambient temperature for 1 h. MeOH (35.7 mg, 1.12 mmol) was added dropwise. The mixture was stirred at ambient temperature for 30 min and was concentrated under reduced pressure to afford 4-[(R)-[2-chloro-3-(2-fluoroethoxy)phenyl]-phenyl-methyl]piperidine hydroiodide as an orange gum (58.0 mg, 55% p., 72% y.). m/z [M+H]⁺=348.3, ¹H NMR (400 MHz, DMSO-d₆): δ (ppm) 8.40 (s, 1H), 8.09 (s, 1H), 7.47 (s, 1H), 7.44-7.28 (m, 4H), 7.28-7.15 (m, 2H), 7.03-6.95 (m, 1H), 4.80 (t, J=3.8 Hz, 1H), 4.68 (t, J=3.8 Hz, 1H), 4.37-4.10 (m, 2H), 4.03 (q, J=7.1 Hz, 1H), 3.23 (s, 3H), 2.86 (d, J=21.2 Hz, 1H), 2.58 (d, J=11.7 Hz, 1H), 1.75-1.40 (m, 2H), 1.38-1.11 (m, 2H).

### Intermediate 101E: tert-butyl(4S)-5-amino-4-[5-[4-[(R)-[2-chloro-3-(2-fluoroethoxy)phenyl]-phenyl-methyl]piperidine-1-carbonyl]-6-methyl-1-oxo-isoindolin-2-yl]-5-oxo-pentanoate

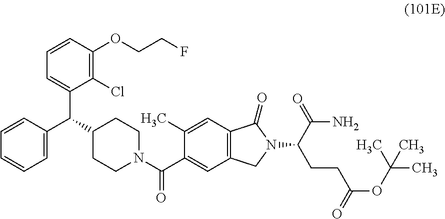 [SMILES: Cc1cc2c(cc1C(=O)N1CCC([C@H](c3ccccc3)c3cccc(OCCF)c3Cl)CC1)CN([C@@H](CCC(=O)OC(C)(C)C)C(N)=O)C2=O]

To a solution of 4-[(R)-[2-chloro-3-(2-fluoroethoxy)phenyl]-phenyl-methyl]piperidine hydroiodide (58.0 mg, 0.0670 mmol) in DMF (330 μL), were added 2-[(1S)-4-tert-butoxy-1-carbamoyl-4-oxo-butyl]-6-methyl-1-oxo-isoindoline-5-carboxylic acid (27.8 mg, 0.0738 mmol), DIPEA (58.6 μL, 0.335 mmol), and HATU (39.4 mg, 0.101 mmol). The mixture was stirred at ambient temperature for 2 h. Water, EtOAc and FastWorkX® were added. The organic solvent was evaporated under reduced pressure. The reaction mixture was filtered through a dry-loading cartridge and the resin was rinsed with water. The crude was purified by flash chromatography on silica gel using a gradient of EtOAc/EtOH in cyclohexane from 5% to 100%. The desired fractions were combined and concentrated under reduced pressure to afford tert-butyl(4S)-5-amino-4-[5-[4-[(R)-[2-chloro-3-(2-fluoroethoxy)phenyl]-phenyl-methyl]piperidine-1-carbonyl]-6-methyl-1-oxo-isoindolin-2-yl]-5-oxo-pentanoate as an orange gum (30.0 mg, 63% p., 40% y.). m/z [M−tBu+2H]⁺=650.3.

### Example 101

To a stirred solution of tert-butyl(4S)-5-amino-4-[5-[4-[(R)-[2-chloro-3-(2-fluoroethoxy)phenyl]-phenyl-methyl]piperidine-1-carbonyl]-6-methyl-1-oxo-isoindolin-2-yl]-5-oxo-pentanoate (30.0 mg, 0.0425 mmol) in acetonitrile (430 μL) was added 4-methylbenzenesulfonic acid hydrate (24.2 mg, 0.127 mmol). The reaction mixture was stirred at 80° C. for 5 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure until acetonitrile evaporated. The resulting aqueous suspension was basified with a saturated aqueous solution of NaHCO₃. The resulting suspension was filtered and dried at 50° C. under reduced pressure for 48 hours to afford (3S)-3-[5-[4-[(R)-[2-chloro-3-(2-fluoroethoxy)phenyl]-phenyl-methyl]piperidine-1-carbonyl]-6-methyl-1-oxo-isoindolin-2-yl]piperidine-2,6-dione as a white powder (5.7 mg, 97.8% p., 21% y.). m/z [M+H]⁺=632.4, ¹H NMR (600 MHz, DMSO-d₆): δ (ppm) 10.97 (s, 1H), 7.59 (br s, 1H), 7.50-7.38 (m, 1H), 7.38-7.33 (m, 2H), 7.33-7.31 (m, 1H), 7.31-7.28 (m, 1H), 7.28-7.23 (m, 2H), 7.20-7.09 (m, 1H), 7.03-6.90 (m, 1H), 5.16-5.02 (m, 1H), 4.82-4.63 (m, 2H), 4.51 (br d, J=4.3 Hz, 1H), 4.46-4.36 (m, 1H), 4.35-4.29 (m, 1H), 4.29-4.21 (m, 2H), 4.21-4.16 (m, 1H), 3.27-3.14 (m, 1H), 3.03-2.94 (m, 1H), 2.93-2.83 (m, 1H), 2.78 (s, 1H), 2.64-2.52 (m, 2H), 2.44-2.35 (m, 1H), 2.34-2.17 (m, 3H), 2.04-1.91 (m, 1H), 1.69-1.46 (m, 1H), 1.45-1.27 (m, 1H), 1.20-1.09 (m, 1H), 1.08-0.89 (m, 1H).

### Example 102

### (3S)-3-[5-[4-[(R)-[2-chloro-3-(cyclopropoxy)phenyl]-phenyl-methyl]piperidine-1-carbonyl]-6-methyl-1-oxo-isoindolin-2-yl]piperidine-2,6-dione

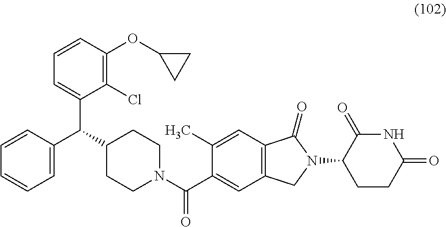 [SMILES: Cc1cc2c(cc1C(=O)N1CCC([C@H](c3ccccc3)c3cccc(OC4CC4)c3Cl)CC1)CN([C@H]1CCC(=O)NC1=O)C2=O]

### Intermediate 102A: cyclopropyl 4-methylbenzenesulfonate

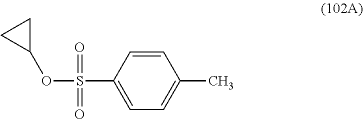

To a stirred solution of cyclopropanol (500 mg, 8.18 mmol) in DCM (13 mL) were added successively triethylamine (1.37 mL, 9.81 mmol) and then DMAP (1.01 g, 8.18 mmol). The reaction mixture was stirred at ambient temperature for 1 h and then 4-methylbenzenesulfonyl chloride (2.07 g, 10.6 mmol) was added. The reaction mixture was stirred at ambient temperature for 16 h. The reaction mixture was concentrated under reduced pressure. The crude was purified by flash chromatography on silica gel using a gradient of EtOAc in cyclohexane from 5% to 80% to afford cyclopropyl 4-methylbenzenesulfonate as a colorless oil (1.49 g, 99% p., 85% y.). m/z [M+H]⁺=no ionization, 1H NMR (400 MHz, DMSO-d₆): δ (ppm) 7.84-7.76 (m, 2H), 7.54-7.46 (m, 2H), 4.06-3.97 (m, 1H), 2.44 (s, 3H), 0.64-0.54 (m, 4H).

### Intermediate 102B: benzyl 4-[(R)-[2-chloro-3-(cyclopropoxy)phenyl]-phenyl-methyl]piperidine-1-carboxylate

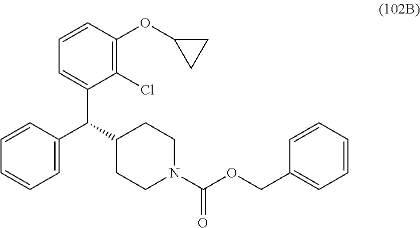 [SMILES: O=C(OCc1ccccc1)N1CCC([C@H](c2ccccc2)c2cccc(OC3CC3)c2Cl)CC1]

To a stirred solution of benzyl 4-[(R)-(2-chloro-3-hydroxy-phenyl)-phenyl-methyl]piperidine-1-carboxylate (175 mg, 0.397 mmol) and cyclopropyl 4-methylbenzenesulfonate (426 mg, 1.99 mmol) were added Cs₂CO₃ (785 mg, 2.38 mmol) and triethylamine (277 μL, 1.99 mmol). The reaction mixture was stirred at 110° C. for 48 h. The reaction was quenched with the addition of water. EtOAc was added. The layers were separated and the aqueous layer was extracted with EtOAc (2×). The combined organic layers were washed with brine, dried using a phase separator cartridge, and concentrated under reduced pressure. The crude was purified by flash chromatography on silica gel using a gradient of EtOAc in n-heptane from 5% to 100% to afford benzyl 4-[(R)-[2-chloro-3-(cyclopropoxy)phenyl]-phenyl-methyl]piperidine-1-carboxylate as a white foam (147 mg, 92% p., 71% y.). m/z [M+H]⁺=476.3, ¹H NMR (400 MHz, DMSO-d₆): δ (ppm) 7.38-7.32 (m, 6H), 7.32-7.20 (m, 6H), 7.16 (t, J=7.3 Hz, 1H), 5.05 (s, 2H), 4.14 (d, J=11.2 Hz, 1H), 3.96 (d, J=12.4 Hz, 2H), 3.86 (tt, J=6.1, 3.0 Hz, 1H), 2.78 (s, 2H), 2.49 (d, J=1.8 Hz, 3H), 1.47 (d, J=12.5 Hz, 1H), 1.37 (d, J=13.4 Hz, 1H), 0.88-0.72 (m, 2H), 0.72-0.56 (m, 2H), [α]_{D}=+49° mL·dm⁻¹ g⁻¹[20° C., Na lamp, 589 nm, C=2.5 mg/mL, EtOH].

### Intermediate 102C: 4-[(R)-[2-chloro-3-(cyclopropoxy)phenyl]-phenyl-methyl]piperidine hydroiodide

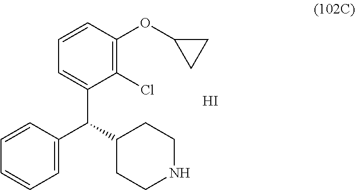

To a stirred solution of benzyl 4-[(R)-[2-chloro-3-(cyclopropoxy)phenyl]-phenyl-methyl]piperidine-1-carboxylate (145 mg, 0.305 mmol) in acetonitrile (3.0 mL) at ambient temperature was added iodo (trimethyl) silane (130 μL, 0.914 mmol) dropwise. The reaction mixture was stirred at ambient temperature for 1 h. MeOH (117 mg, 3.66 mmol) was added dropwise. The mixture was stirred at ambient temperature for 30 min and was concentrated under reduced pressure to afford 4-[(R)-[2-chloro-3-(cyclopropoxy)phenyl]-phenyl-methyl]piperidine hydroiodide as an orange gum (192 mg, 70% p., 94% y.). m/z [M+H]⁺=342.3, ¹H NMR (400 MHz, DMSO-d₆): δ (ppm) 8.39 (s, 1H), 8.09 (s, 1H), 7.50-7.39 (m, 1H), 7.39-7.21 (m, 7H), 4.62 (s, 1H), 4.14 (d, J=11.2 Hz, 1H), 3.87 (tt, J=6.1, 2.9 Hz, 1H), 3.23 (d, J=11.9 Hz, 2H), 2.91-2.78 (m, 2H), 2.58 (d, J=11.4 Hz, 1H), 1.99 (s, 1H), 1.55 (dd, J=26.4, 14.3 Hz, 2H), 1.33-1.20 (m, 2H), 0.82-0.78 (m, 1H), 0.66 (h, J=3.8 Hz, 1H).

### Intermediate 102D: tert-butyl(4S)-5-amino-4-[5-[4-[(R)-[2-chloro-3-(cyclopropoxy)-phenyl]phenyl-methyl]piperidine-1-carbonyl]-6-methyl-1-oxo-isoindolin-2-yl]-5-oxo-pentanoate

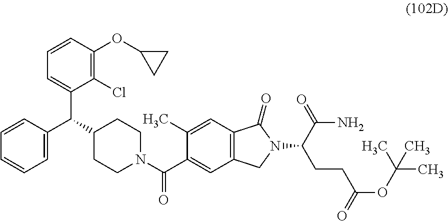 [SMILES: Cc1cc2c(cc1C(=O)N1CCC([C@H](c3ccccc3)c3cccc(OC4CC4)c3Cl)CC1)CN([C@@H](CCC(=O)OC(C)(C)C)C(N)=O)C2=O]

To a solution of 4-[(R)-[2-chloro-3-(cyclopropoxy)phenyl]-phenyl-methyl]piperidine hydroiodide (190 mg, 0.283 mmol) in DMF (1.4 mL), were added 2-[(1S)-4-tert-butoxy-1-carbamoyl-4-oxo-butyl]-6-methyl-1-oxo-isoindoline-5-carboxylic acid (117 mg, 0.311 mmol), DIPEA (247 μL, 1.42 mmol), and HATU (166 mg, 0.425 mmol). The mixture was stirred at ambient temperature for 2 h. Water, EtOAc and FastWorkX® were added. The organic solvent was evaporated under reduced pressure. The reaction mixture was filtered through a dry-loading cartridge and the resin was rinsed with water. The crude was purified by flash chromatography on silica gel using a gradient of

EtOAc/EtOH in cyclohexane from 5% to 100%. The desired fractions were combined and concentrated under reduced pressure to afford tert-butyl(4S)-5-amino-4-[5-[4-[(R)-[2-chloro-3-(cyclopropoxy)phenyl]-phenyl-methyl]piperidine-1-carbonyl]-6-methyl-1-oxo-isoindolin-2-yl]-5-oxo-pentanoate as an orange foam (121 mg, 80% p., 49% y.). m/z [M-tBu+2H]⁺=644.4.

### Example 102

To a stirred solution of tert-butyl(4S)-5-amino-4-[5-[4-[(R)-[2-chloro-3-(cyclopropoxy)phenyl]-phenyl-methyl]piperidine-1-carbonyl]-6-methyl-1-oxo-isoindolin-2-yl]-5-oxo-pentanoate (120 mg, 0.171 mmol) in acetonitrile (1.7 mL) was added 4-methylbenzenesulfonic acid hydrate (97.8 mg, 0.514 mmol). The reaction mixture was stirred at 80° C. for 5 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure until acetonitrile evaporated. The resulting aqueous suspension was basified with a saturated aqueous solution of NaHCO₃. The resulting suspension was filtered and dried at 50° C. under reduced pressure for 48 hours to afford (3S)-3-[5-[4-[(R)-[2-chloro-3-(cyclopropoxy)phenyl]-phenyl-methyl]piperidine-1-carbonyl]-6-methyl-1-oxo-isoindolin-2-yl]piperidine-2,6-dione as a white powder (39.6 mg, 99.2% p., 37% y.). m/z [M+H]⁺=626.5, ¹H NMR (500 MHz, DMSO-d₆): δ (ppm) 11.14-10.72 (m, 1H), 7.65-7.56 (m, 1H), 7.51-7.08 (m, 9H), 5.24-4.94 (m, 1H), 4.55-4.24 (m, 3H), 4.23-4.08 (m, 1H), 3.79 (s, 1H), 3.28-2.53 (m, 6H), 2.44-2.16 (m, 4H), 2.04-1.91 (m, 1H), 1.66-0.92 (m, 4H), 0.87-0.55 (m, 4H), [α]_{D}=+7° mL·dm⁻¹ g⁻¹[20° C., Na lamp, 589 nm, C=1.6 mg/mL, DMSO].

### Example 103

### 3-[5-[4-[(R)-(2-chlorophenyl)-phenyl-methyl]piperidine-1-carbonyl]-6-methyl-1-oxo-isoindolin-2-yl]piperidine-2,6-dione

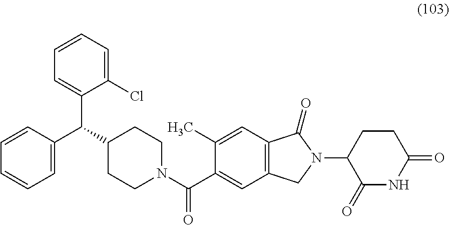 [SMILES: Cc1cc2c(cc1C(=O)N1CCC([C@H](c3ccccc3)c3ccccc3Cl)CC1)CN(C1CCC(=O)NC1=O)C2=O]

### Intermediate 103A: tert-butyl 5-amino-4-[5-[4-[(R)-(2-chlorophenyl)-phenyl-methyl]piperidine-1-carbonyl]-6-methyl-1-oxo-isoindolin-2-yl]-5-oxo-pentanoate

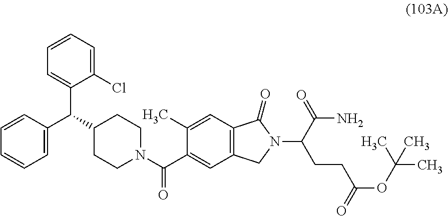 [SMILES: Cc1cc2c(cc1C(=O)N1CCC([C@H](c3ccccc3)c3ccccc3Cl)CC1)CN(C(CCC(=O)OC(C)(C)C)C(N)=O)C2=O]

To a stirred solution of 2-[(1R)-4-tert-butoxy-1-carbamoyl-4-oxo-butyl]-6-methyl-1-oxo-isoindoline-5-carboxylic acid (105 mg, 0.279 mmol) and 2-[(1S)-4-tert-butoxy-1-carbamoyl-4-oxo-butyl]-6-methyl-1-oxo-isoindoline-5-carboxylic acid (105 mg, 0.279 mmol) in DCM (5.6 mL) were added successively 4-[(R)-(2-chlorophenyl)-phenyl-methyl]piperidine hydroiodide (231 mg, 0.559 mmol), DIPEA (244 μL, 1.40 mmol), and a solution of T3P in DCM (53% w/w, 235 μL, 0.419 mmol). The reaction mixture was stirred for 2 h. The reaction was quenched with the addition of water. The aqueous layer was extracted with EtOAc (3×). The combined organic layers were washed with brine, dried over Na₂SO₄, filtered, and concentrated under reduced pressure. The crude was purified by reverse-phase chromatography using a gradient of acetonitrile in water from 10% to 100% (0.1% AcOH). The desired fractions were combined, and acetonitrile removed under reduced pressure. The aqueous layer was basified with a saturated aqueous solution of NaHCO₃. The aqueous layer was extracted with EtOAc (3×). The combined organic layers were washed with brine, dried over Na₂SO₄, filtered, and concentrated under reduced pressure to afford tert-butyl 5-amino-4-[5-[4-[(R)-(2-chlorophenyl)-phenyl-methyl]piperidine-1-carbonyl]-6-methyl-1-oxo-isoindolin-2-yl]-5-oxo-pentanoate as an off-white solid (143 mg, 99% p., 39% y.). m/z [M+H]⁺=644.4.

### Example 103

To a stirred solution of tert-butyl 5-amino-4-[5-[4-[(R)-(2-chlorophenyl)-phenyl-methyl]piperidine-1-carbonyl]-6-methyl-1-oxo-isoindolin-2-yl]-5-oxo-pentanoate (143 mg, 0.222 mmol) in acetonitrile (2.2 mL) was added 4-methylbenzenesulfonic acid hydrate (211 mg, 1.11 mmol). The reaction mixture was stirred at 80° C. for 4 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). Acetonitrile was removed under reduced pressure. The aqueous layer was basified with a saturated aqueous solution of NaHCO₃. The precipitate was triturated for 15 min, filtered, washed with water, and dried under reduced pressure at 50° C. overnight to afford 3-[5-[4-[(R)-(2-chlorophenyl)-phenyl-methyl]piperidine-1-carbonyl]-6-methyl-1-oxo-isoindolin-2-yl]piperidine-2,6-dione as a white solid (73.4 mg, 99.0% p., 57% y.). m/z [M+H]⁺=570.0. ¹H NMR (600 MHz, DMSO-d₆): δ (ppm) 10.97 (s, 1H), 7.83-7.64 (m, 1H), 7.60 (br d, J=7.6 Hz, 1H), 7.52-7.04 (m, 9H), 5.09 (br dd, J=13.1, 4.8 Hz, 1H), 4.52 (br d, J=1.0 Hz, 1H), 4.47-4.36 (m, 1H), 4.35-4.22 (m, 1H), 4.19-4.09 (m, 1H), 3.23 (br d, J=13.6 Hz, 1H), 3.07-2.68 (m, 3H), 2.68-2.53 (m, 2H), 2.47-2.17 (m, 4H), 2.04-1.87 (m, 1H), 1.73-0.64 (m, 4H).

### Example 104

### (3S)-3-[6-methyl-1-oxo-5-[4-[rel-(S)-phenyl(1,2,3,4-tetrahydroquinolin-5-yl)methyl]piperidine-1-carbonyl]isoindolin-2-yl]piperidine-2,6-dione

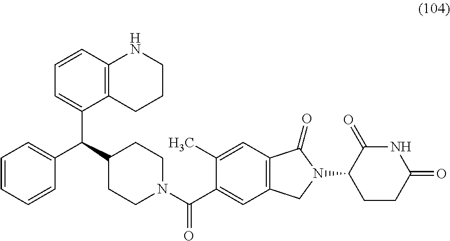 [SMILES: Cc1cc2c(cc1C(=O)N1CCC([C@@H](c3ccccc3)c3cccc4c3CCCN4)CC1)CN([C@H]1CCC(=O)NC1=O)C2=O]

### Intermediate 104A: tert-butyl 4-[phenyl(4,4,5,5-tetramethyl-1,3,2-dioxaborolan-2-yl)methylene]piperidine-1-carboxylate

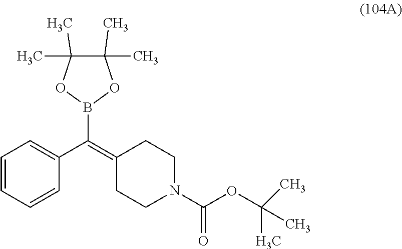 [SMILES: CC(C)(C)OC(=O)N1CCC(=C(B2OC(C)(C)C(C)(C)O2)c2ccccc2)CC1]

To a stirred solution of tert-butyl 4-[bromo (phenyl)methylene]piperidine-1-carboxylate (2.50 g, 7.10 mmol) and 2-isopropoxy-4,4,5,5-tetramethyl-1,3,2-dioxaborolane (2.17 mL, 10.6 mmol) in anhydrous THF (23.7 mL) at −78° C. was added 1.6 M n-butyllithium in hexanes (6.65 mL, 10.6 mmol) dropwise. The reaction mixture was stirred at −78° C. for 30 min. Additional 2-isopropoxy-4,4,5,5-tetramethyl-1,3,2-dioxaborolane (2.90 mL, 14.2 mmol) and then 1.6 M n-butyllithium in hexanes (8.87 mL, 14.2 mmol) were added dropwise. The reaction mixture was stirred at −78° C. for 30 min. Additional 2-isopropoxy-4,4,5,5-tetramethyl-1,3,2-dioxaborolane (2.90 mL, 14.2 mmol) and then 1.6M n-butyllithium in hexanes (8.87 mL, 14.2 mmol) were added. The reaction mixture was stirred at −78° C. for 30 min. The reaction was quenched with the addition of a saturated aqueous solution of NH₄C1. The aqueous layer was extracted with EtOAc (3×). The combined organic layers were washed with brine, dried over Na₂SO₄, filtered, and concentrated under reduced pressure. The crude was purified by flash chromatography on silica using a gradient of EtOAc in n-heptane from 1% to 40% to afford (tert-butyl 4-[phenyl-(4,4,5,5-tetramethyl-1,3,2-dioxaborolan-2-yl)methylene]piperidine-1-carboxylate as a white solid (1.43 g, 3.54 mmol, 99% p., 50% y.). m/z [M+H]⁺=300.3, ¹H NMR (400 MHz, DMSO-d₆): δ (ppm) 7.34-7.21 (m, 2H), 7.22-7.11 (m, 1H), 7.08-6.91 (m, 2H), 3.42 (t, J=5.9 Hz, 2H), 3.28 (t, J=5.9 Hz, 2H), 2.64-2.55 (m, 2H), 2.14-2.01 (m, 2H), 1.40 (s, 9H), 1.19 (s, 12H).

### Intermediate 104B: tert-butyl 5-[(1-tert-butoxycarbonyl-4-piperidylidene)-phenyl-methyl]-3,4-dihydro-2H-quinoline-1-carboxylate

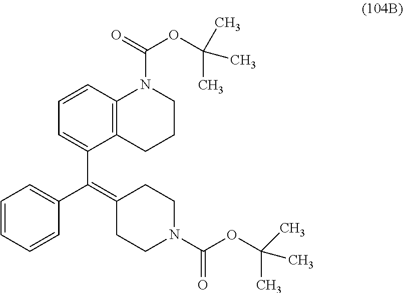 [SMILES: CC(C)(C)OC(=O)N1CCC(=C(c2ccccc2)c2cccc3c2CCCN3C(=O)OC(C)(C)C)CC1]

To a stirred solution of tert-butyl 4-[phenyl-(4,4,5,5-tetramethyl-1,3,2-dioxaborolan-2-yl)methylene]piperidine-1-carboxylate (400 mg, 1.00 mmol) and tert-butyl 5-bromo-1,2,3,4-tetrahydroquinoline-1-carboxylate (375 mg, 1.20 mmol) in CPME (4.0 mL) and water (1.0 mL) was added K₂CO₃ (415 mg, 3.01 mmol). The reaction mixture was degassed with argon for 5 min then Pd(PPh3) 4 (116 mg, 0.100 mmol) was added. The reaction mixture was stirred at 110° C. overnight. The reaction was quenched with the addition of a saturated aqueous solution of NH₄Cl. EtOAc was added. The layers were separated, and the aqueous layer extracted with EtOAc (2×). The combined organic layers were washed with brine, dried using hydrophobic paper and concentrated under reduced pressure. The crude was purified by flash chromatography on silica gel using a gradient of EtOAc in n-heptane from 0% to 50% to afford tert-butyl 5-[(1-tert-butoxycarbonyl-4-piperidylidene)-phenyl-methyl]-3,4-dihydro-2H-quinoline-1-carboxylate as an orange gum (354 mg, 100% p., 70% y.). m/z [M+Na]⁺=527.3. ¹H NMR (400 MHz, DMSO-d₆): δ (ppm) 7.45 (dd, J=8.3, 1.2 Hz, 1H), 7.31 (dd, J=8.1, 6.8 Hz, 2H), 7.25-7.18 (m, 1H), 7.18-7.07 (m, 3H), 6.88 (dd, J=7.5, 1.2 Hz, 1H), 3.48 (dd, J=13.2, 6.4 Hz, 1H), 3.42-3.32 (m, 2H), 2.62 (dt, J=16.3, 6.4 Hz, 1H), 2.41-2.30 (m, 2H), 2.25 (ddd, J=13.8, 6.9, 4.2 Hz, 1H), 2.07-1.90 (m, 2H), 1.73 (p, J=6.4 Hz, 2H), 1.45 (s, 9H), 1.39 (s, 9H), 1.32-1.12 (m, 3H).

### Intermediate 104C: tert-butyl 5-[(1-tert-butoxycarbonyl-4-piperidyl)-phenyl-methyl]-3,4-dihydro-2H-quinoline-1-carboxylate

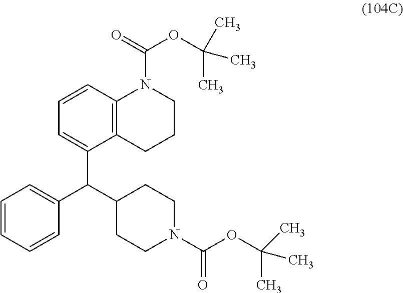 [SMILES: CC(C)(C)OC(=O)N1CCC(C(c2ccccc2)c2cccc3c2CCCN3C(=O)OC(C)(C)C)CC1]

In a sealed vial, a stirred solution of tert-butyl 5-[(1-tert-butoxycarbonyl-4-piperidylidene)-phenyl-methyl]-3,4-dihydro-2H-quinoline-1-carboxylate (350 mg, 0.694 mmol) in EtOH (6.9 mL) was degassed with argon for 5 min and palladium on carbon (10% w/w, 148 mg, 0.139 mmol) and triethylsilane (554 μL, 3.47 mmol) were added. The mixture was stirred at 70° C. overnight and then cooled down to ambient temperature. The suspension was filtered through a glass fiber filter and rinsed with EtOAc. The filtrate concentrated under reduced pressure to afford tert-butyl 5-[(1-tert-butoxycarbonyl-4-piperidyl)-phenyl-methyl]-3,4-dihydro-2H-quinoline-1-carboxylate as a pale yellow oil (449 mg, 99% p., quant. yield)). m/z [M+Na]⁺=529.5.

### Intermediate 104D: 5-[phenyl(4-piperidyl)methyl]-1,2,3,4-tetrahydroquinoline dihydrochloride

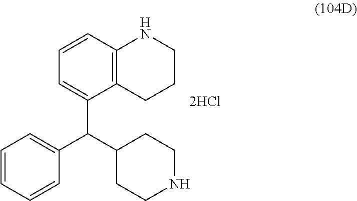 [SMILES: c1ccc(C(c2cccc3c2CCCN3)C2CCNCC2)cc1]

To a stirred solution of tert-butyl 5-[(1-tert-butoxycarbonyl-4-piperidyl)-phenyl-methyl]-3,4-dihydro-2H-quinoline-1-carboxylate (445 mg, 0.878 mmol) in 1,4-dioxane (4.4 mL) was added a 4 M solution of HCl in 1,4-dioxane (4.4 mL, 17.6 mmol) dropwise. The reaction mixture was stirred overnight at ambient temperature. Et₂O(88 mL) was added and the reaction mixture was stirred for 1 h. The suspension was filtered, washed with Et₂O, and dried under reduced pressure to afford 5-[phenyl(4-piperidyl)methyl]-1,2,3,4-tetrahydroquinoline dihydrochloride as a white solid (208 mg, 99% p., 62% y.). m/z [M+H]⁺=307.4.

### Intermediate 104E: tert-butyl(4S)-5-amino-4-[6-methyl-1-oxo-5-[4-[phenyl(1,2,3,4-tetrahydroquinolin-5-yl)methyl]piperidine-1-carbonyl]isoindolin-2-yl]-5-oxo-pentanoate

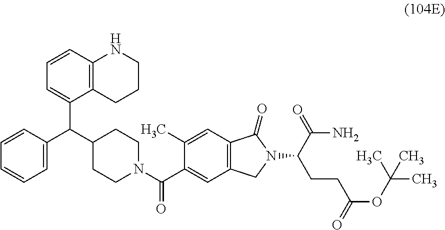 [SMILES: Cc1cc2c(cc1C(=O)N1CCC(C(c3ccccc3)c3cccc4c3CCCN4)CC1)CN([C@@H](CCC(=O)OC(C)(C)C)C(N)=O)C2=O]

To a stirred solution of 5-[phenyl(4-piperidyl)methyl]-1,2,3,4-tetrahydroquinoline dihydrochloride (207 mg, 0.545 mmol) in DCM (5.4 mL) were added successively DIPEA (476 μL, 2.72 mmol), 2-[(1S)-4-tert-butoxy-1-carbamoyl-4-oxo-butyl]-6-methyl-1-oxo-isoindoline-5-carboxylic acid (205 mg, 0.545 mmol), and then a solution of T3P in DCM (53% w/w, 459 μL, 0.817 mmol). The reaction mixture was stirred for 2 h. The reaction was quenched with the addition of water. The aqueous layer was extracted with EtOAc (3×). The combined organic layers were washed with brine, dried over Na₂SO₄, filtered, and concentrated under reduced pressure. The crude was purified by reverse-phase chromatography using a gradient of acetonitrile in water from 10% to 100% (0.1% AcOH). The desired fractions were combined and acetonitrile removed under reduced pressure. The aqueous layer was basified with a saturated aqueous solution of NaHCO₃. The aqueous layer was extracted with EtOAc (3×). The combined organic layers were washed brine, dried over Na₂SO₄, filtered, and concentrated under reduced pressure to afford tert-butyl(4S)-5-amino-4-[6-methyl-1-oxo-5-[4-[phenyl(1,2,3,4-tetrahydroquinolin-5-yl)methyl]piperidine-1-carbonyl]isoindolin-2-yl]-5-oxo-pentanoate as an off-white solid (195 mg, 98% p., 53% y.). m/z [M+H]⁺=665.5.

### Intermediate 104F: tert-butyl(4S)-5-amino-4-[6-methyl-1-oxo-5-[4-[rel-(S)-phenyl(1,2,3,4-tetrahydroquinolin-5-yl)methyl]piperidine-1-carbonyl]isoindolin-2-yl]-5-oxo-pentanoate

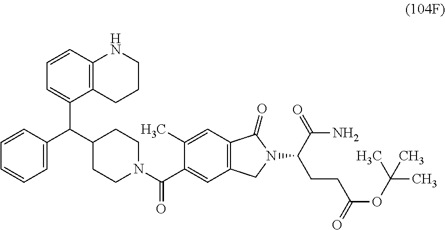 [SMILES: Cc1cc2c(cc1C(=O)N1CCC(C(c3ccccc3)c3cccc4c3CCCN4)CC1)CN([C@@H](CCC(=O)OC(C)(C)C)C(N)=O)C2=O]

tert-butyl(4S)-5-amino-4-[6-methyl-1-oxo-5-[4-[phenyl(1,2,3,4-tetrahydroquinolin-5-yl)methyl]piperidine-1-carbonyl]isoindolin-2-yl]-5-oxo-pentanoate was purified by chiral supercritical chromatography (Waters Prep SFC80, Chiralpak IB (5 μm, 250×20 mm), CO₂/(EtOH/acetonitrile (7/3)) 77/23 as mobile phase, flow rate 50 mL/min, 100 bar) to afford (second isomer eluted) (tert-butyl(4S)-5-amino-4-[6-methyl-1-oxo-5-[4-[rel-(S)-phenyl(1,2,3,4-tetrahydroquinolin-5-yl)methyl]piperidine-1-carbonyl]isoindolin-2-yl]-5-oxo-pentanoate (49.1 mg, 0.0739 mmol, 100% p., 32% y.) as a white solid. m/z [M+H]⁺=665.5. ¹H NMR (400 MHz, DMSO-d₆): δ (ppm) 7.73-7.49 (m, 2H), 7.45-6.96 (m, 7H), 6.84 (dt, J=23.1, 7.8 Hz, 1H), 6.71 (dd, J=17.9, 7.7 Hz, 1H), 6.25 (dd, J=20.9, 7.9 Hz, 1H), 5.50 (d, J=21.1 Hz, 1H), 4.72 (d, J=10.5 Hz, 1H), 4.66-4.20 (m, 3H), 3.74 (d, J=10.5 Hz, 1H), 3.26-2.51 (m, 7H), 2.45-2.04 (m, 7H), 1.97 (s, 1H), 1.75 (s, 2H), 1.63-0.66 (m, 13H).

### Example 104

To a stirred solution of tert-butyl(4S)-5-amino-4-[6-methyl-1-oxo-5-[4-[rel-(S)-phenyl(1,2,3,4-tetrahydroquinolin-5-yl)methyl]piperidine-1-carbonyl]isoindolin-2-yl]-5-oxo-pentanoate (49.0 mg, 0.0737 mmol) in acetonitrile (740 μL) was added 4-methylbenzenesulfonic acid hydrate (70.1 mg, 0.369 mmol). The reaction mixture was stirred at 80° C. for 4 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). Acetonitrile was removed under reduced pressure. The aqueous layer was basified with a saturated aqueous solution of NaHCO₃. The precipitate was triturated for 15 min, filtered, washed with water, and dried under reduced pressure at 50° C. overnight to afford (3S)-3-[6-methyl-1-oxo-5-[4-[rel-(S)-phenyl (1,2,3,4-tetrahydroquinolin-5-yl)methyl]piperidine-1-carbonyl]isoindolin-2-yl]piperidine-2,6-dione as a white solid (23.9 mg, 98.0% p., 54% y.). m/z [M+H]⁺=591.0. ¹H NMR (600 MHz, DMSO-d₆): δ (ppm) 11.01-10.89 (m, 1H), 7.60 (br d, J=9.0 Hz, 1H), 7.48-7.32 (m, 1H), 7.32-7.26 (m, 2H), 7.26-7.18 (m, 2H), 7.17-7.04 (m, 1H), 6.91-6.76 (m, 1H), 6.75-6.63 (m, 1H), 6.27 (dd, J=7.9, 1.0 Hz, 1H), 5.56-5.39 (m, 1H), 5.10 (dd, J=13.4, 5.1 Hz, 1H), 4.56-4.46 (m, 1H), 4.44-4.21 (m, 2H), 3.81-3.69 (m, 1H), 3.27-3.14 (m, 1H), 3.12-3.00 (m, 2H), 2.94 (s, 1H), 2.91-2.87 (m, 1H), 2.78 (s, 1H), 2.77-2.65 (m, 1H), 2.63-2.57 (m, 1H), 2.57-2.51 (m, 1H), 2.44-2.38 (m, 1H), 2.38-2.35 (m, 1H), 2.24 (s, 3H), 1.97 (br d, J=18.9 Hz, 1H), 1.88-1.73 (m, 2H), 1.72-1.50 (m, 1H), 1.44-1.20 (m, 1H), 1.18-1.07 (m, 1H), 1.07-0.93 (m, 1H) [α]_{D}=−42°. mL·dm⁻¹ g⁻¹[20° C., Na lamp, 589 nm, C=1.8 mg/mL, DMSO].

### Example 105

### (3S)-3-[5-[4-[(2-chloro-3-pyridyl)-phenyl-methyl]piperidine-1-carbonyl]-6-methyl-1-oxo-isoindolin-2-yl]piperidine-2,6-dione

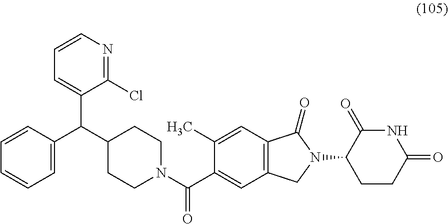 [SMILES: Cc1cc2c(cc1C(=O)N1CCC(C(c3ccccc3)c3cccnc3Cl)CC1)CN([C@H]1CCC(=O)NC1=O)C2=O]

### Intermediate 105A: tert-butyl 4-[(2-benzyloxy-3-pyridyl)-phenyl-methylene]piperidine-1-carboxylate

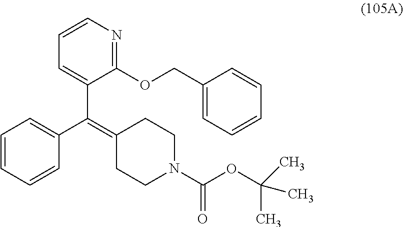 [SMILES: CC(C)(C)OC(=O)N1CCC(=C(c2ccccc2)c2cccnc2OCc2ccccc2)CC1]

In a sealed vial, to a stirred solution of tert-butyl 4-[bromo (phenyl)methylene]piperidine-1-carboxylate (250 mg, 0.710 mmol) in 1,4-dioxane (2.5 mL) and water (0.75 mL) were added under argon, [2-(benzyloxy)pyridin-3-yl]boronic acid (163 mg, 0.710 mmol) and K₃PO₄ (458 mg, 2.13 mmol). The reaction mixture was degassed with argon for 5 min after which Pd(PPh3) 4 (83.7 mg, 0.0710 mmol) was added. The reaction mixture was stirred at 90° C. overnight. The reaction mixture was partitioned between EtOAc and water. The layers were separated, and the aqueous layer extracted with EtOAc (2×). The combined organic layers were washed with brine, dried over Na₂SO₄, filtered, and concentrated under reduced pressure. The crude was purified by flash chromatography on silica gel using a gradient of EtOAc in n-heptane from 0% to 50%. The desired fractions were combined, concentrated, and dried under reduced pressure to afford tert-butyl 4-[(2-benzyloxy-3-pyridyl)-phenyl-methylene]piperidine-1-carboxylate as a colorless oil (208 mg, 100% p., 64% y.). m/z [M+H]⁺=457.3. ¹H NMR (400 MHz, DMSO-d₆): δ (ppm) 8.08 (dd, J=5.0, 1.9 Hz, 1H), 7.55 (dd, J=7.2, 1.9 Hz, 1H), 7.33-7.19 (m, 6H), 7.13 (ddt, J=10.7, 5.9, 1.7 Hz, 4H), 7.00 (dd, J=7.2, 5.0 Hz, 1H), 5.30 (d, J=20.1 Hz, 2H), 3.24 (s, 4H), 2.25 (s, 2H), 1.99 (s, 2H), 1.38 (s, 9H).

### Intermediate 105B: tert-butyl 4-[(2-oxo-1H-pyridin-3-yl)-phenyl-methyl]piperidine-1-carboxylate

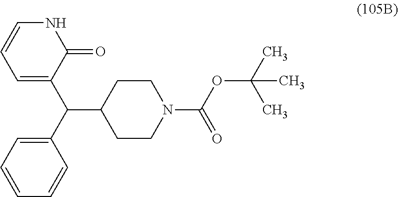 [SMILES: CC(C)(C)OC(=O)N1CCC(C(c2ccccc2)c2ccc[nH]c2=O)CC1]

In a sealed vial, to stirred suspension of tert-butyl 4-[(2-benzyloxy-3-pyridyl)-phenyl-methylene]piperidine-1-carboxylate (207 mg, 0.453 mmol) in EtOAc (4.5 mL) under argon was added palladium on carbon (10% w/w, 241 mg, 0.227 mmol and triethylsilane (293 μL, 1.81 mmol). The suspension was stirred for 3.3 h at 70° C. The reaction mixture was filtered through a fiber glass filter and concentrated under reduced pressure. The crude was purified by flash chromatography on silica gel using a gradient of EtOAc/EtOH (3/1) in n-heptane from 0% to 100%. The desired fractions were combined, concentrated, and dried under reduced pressure to afford tert-butyl 4-[(2-oxo-1H-pyridin-3-yl)-phenyl-methyl]piperidine-1-carboxylate as a colorless oil (51.0 mg, 78% p., 24% y.). m/z [M+H]⁺=369.4.

### Intermediate 105C: 2-chloro-3-[phenyl(4-piperidyl)methyl]pyridine

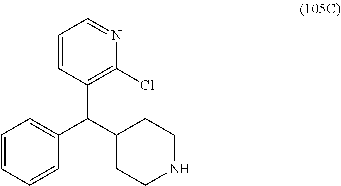 [SMILES: Clc1ncccc1C(c1ccccc1)C1CCNCC1]

In a sealed vial, was combined tert-butyl 4-[(2-oxo-1H-pyridin-3-yl)-phenyl-methyl]piperidine-1-carboxylate (80.0 mg, 0.169 mmol), toluene, (1.7 mL) and phosphoryl trichloride (47.4 μL, 0.508 mmol). The reaction mixture was stirred at 110° C. overnight. The reaction mixture was concentrated under reduced pressure to afford 2-chloro-3-[phenyl(4-piperidyl)methyl]pyridine as a colorless oil (31.0 mg, 39% p., 25% y.). m/z [M+H]⁺=287.2.

### Intermediate 105D: tert-butyl(4S)-5-amino-4-[5-[4-[(2-chloro-3-pyridyl)-phenyl-methyl]piperidine-1-carbonyl]-6-methyl-1-oxo-isoindolin-2-yl]-5-oxo-pentanoate

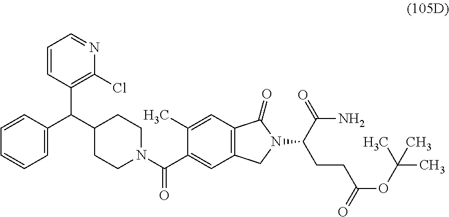 [SMILES: Cc1cc2c(cc1C(=O)N1CCC(C(c3ccccc3)c3cccnc3Cl)CC1)CN([C@@H](CCC(=O)OC(C)(C)C)C(N)=O)C2=O]

In a round-bottom flask, was combined 6-methyl-1-oxo-2-[rac-(1S)-4-tert-butoxy-1-carbamoyl-4-oxo-butyl]isoindoline-5-carboxylic acid (30.8 mg, 0.0803 mmol) and 2-chloro-3-[phenyl(4-piperidyl)methyl]pyridine (31.0 mg, 0.0530 mmol) in dry DCM (220 μL). Then, DIPEA (54.7 μL, 0.313 mmol) was added, followed by the addition of a solution of T3P 53% in DCM (44.6 μL, 0.0794 mmol) at 0° C. The reaction mixture was stirred at 0° C. for 30 min. The reaction was quenched with the addition of a saturated aqueous solution of NH₄Cl. DCM was added. The layers were separated and the aqueous layer extracted with DCM. The combined organic layers were washed with brine, dried over Na₂SO₄, filtered, and concentrated under reduced pressure. The crude was purified by flash chromatography on silica gel using a gradient of EtOAc/EtOH (3/1) in n-heptane from 5% to 100% to afford tert-butyl(4S)-5-amino-4-[5-[4-[(2-chloro-3-pyridyl)-phenyl-methyl]piperidine-1-carbonyl]-6-methyl-1-oxo-isoindolin-2-yl]-5-oxo-pentanoate as a white solid (17.0 mg, 94% p., 47% y.). m/z [M+H]⁺=645.5, ¹H NMR (400 MHz, DMSO-d₆): δ (ppm) 8.33-8.13 (m, 2H), 7.62-7.07 (m, 10H), 4.77-4.67 (m, 1H), 4.54 (d, J=15.7 Hz, 2H), 4.42 (d, J=16.3 Hz, 1H), 4.07 (d, J=11.0 Hz, 1H), 3.25 (d, J=15.1 Hz, 1H), 2.98 (dd, J=24.4, 12.5 Hz, 1H), 2.85-2.53 (m, 2H), 2.33 (s, 1H), 2.24 (s, 1H), 2.14 (s, 3H), 2.04-1.88 (m, 1H), 1.64-1.11 (m, 14H).

### Example 105

To a stirred solution of tert-butyl(4S)-5-amino-4-[5-[4-[(2-chloro-3-pyridyl)-phenyl-methyl]piperidine-1-carbonyl]-6-methyl-1-oxo-isoindolin-2-yl]-5-oxo-pentanoate (16.0 mg, 0.0233 mmol) in dry acetonitrile (1.5 mL) was added 4-methylbenzenesulfonic acid hydrate (17.7 mg, 0.0932 mmol). The reaction mixture was stirred at 90° C. for 1.5 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure until evaporation of the acetonitrile. A saturated aqueous solution of NaHCO₃ was added. The resulting precipitate was filtered and dried under reduced pressure at 50° C. overnight to afford (3S)-3-[5-[4-[(2-chloro-3-pyridyl)-phenyl-methyl]piperidine-1-carbonyl]-6-methyl-1-oxo-isoindolin-2-yl]piperidine-2,6-dione as a white powder (4.50 mg, 99.3% p., 34% y.). m/z [M+H]⁺=571.4. ¹H NMR (600 MHz, DMSO-d₆): δ (ppm) 10.97 (s, 1H), 8.30-8.15 (m, 2H), 7.63-7.54 (m, 1H), 7.51-7.23 (m, 6H), 7.23-7.11 (m, 1H), 5.13-5.02 (m, 1H), 4.59-4.19 (m, 3H), 4.11-4.03 (m, 1H), 3.29-2.54 (m, 6H), 2.44-2.19 (m, 4H), 2.07-1.90 (m, 1H), 1.65-0.82 (m, 4H).

### Example 106

### (3S)-3-[6-methyl-1-oxo-5-[4-[phenyl(2,3,4,5-tetrahydro-1H-1-benzazepin-6-yl)methyl]piperidine-1-carbonyl]isoindolin-2-yl]piperidine-2,6-dione

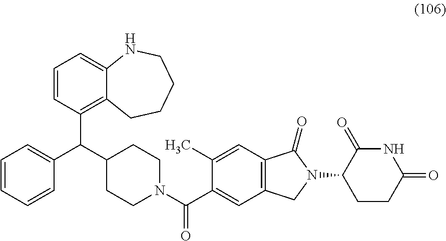 [SMILES: Cc1cc2c(cc1C(=O)N1CCC(C(c3ccccc3)c3cccc4c3CCCCN4)CC1)CN([C@H]1CCC(=O)NC1=O)C2=O]

### Intermediate 106A: tert-butyl 4-[phenyl(2,3,4,5-tetrahydro-1H-1-benzazepin-6-yl)methylene]piperidine-1-carboxylate

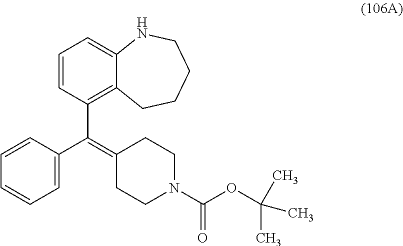 [SMILES: CC(C)(C)OC(=O)N1CCC(=C(c2ccccc2)c2cccc3c2CCCCN3)CC1]

To a stirred solution of tert-butyl 4-[bromo (phenyl)methylene]piperidine-1-carboxylate (500 mg, 1.42 mmol) in CPME (9.0 mL) were added B₂pin₂(396 mg, 1.56 mmol) and Cs₂CO₃ (2.77 g, 8.52 mmol). The reaction mixture was degassed with argon for 5 min. Pd(PPh3) 4 (49.2 mg, 0.0426 mmol) was added and the reaction mixture was stirred at 80° C. for 2 h. The reaction mixture was cooled to ambient temperature. A solution of 6-bromo-2,3,4,5-tetrahydro-1H-1-benzazepine (353 mg, 1.56 mmol) in CPME (3.0 mL) and water (3.0 mL) were added and the reaction mixture was stirred at 110° C. overnight. The reaction mixture was cooled to ambient temperature and diluted with water. The aqueous layer was extracted with EtOAc (3×). The combined organic layers were washed with brine, dried over Na₂SO₄, filtered, and concentrated under reduced pressure. The crude was purified by flash chromatography on silica gel using a gradient of EtOAc/EtOH (3/1) in n-heptane from 1% to 100% to afford tert-butyl 4-[phenyl(2,3,4,5-tetrahydro-1H-1-benzazepin-6-yl)methylene]piperidine-1-carboxylate as a pale yellow solid (455 mg, 53% p., 41% y.). m/z [M+H]⁺=419.5.

### Intermediate 106B: tert-butyl 4-[phenyl(2,3,4,5-tetrahydro-1H-1-benzazepin-6-yl)methyl]piperidine-1-carboxylate

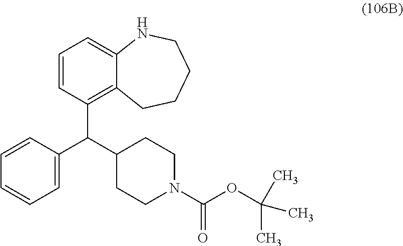 [SMILES: CC(C)(C)OC(=O)N1CCC(C(c2ccccc2)c2cccc3c2CCCCN3)CC1]

In a sealed vial, a stirred solution of tert-butyl 4-[phenyl(2,3,4,5-tetrahydro-1H-1-benzazepin-6-yl)methylene]piperidine-1-carboxylate (455 mg, 1.09 mmol) in EtOAc (7.2 mL) was degassed with argon for 5 min and palladium on carbon (231 mg, 0.217 mmol) and triethylsilane (868 μL, 5.44 mmol) were added. The mixture was stirred at 70° C. overnight and then cooled down to ambient temperature. Additional AcOH (622 μL, 10.9 mmol), palladium on carbon (1.16 g, 1.09 mmol), and then triethylsilane (868 μL, 5.44 mmol) were added. The mixture was stirred at 70° C. overnight and then cooled down to ambient temperature. The suspension was filtered over glass fiber and rinsed with EtOAc. The filtrate was concentrated under reduced pressure to afford tert-butyl 4-[phenyl(2,3,4,5-tetrahydro-1H-1-benzazepin-6-yl)methyl]piperidine-1-carboxylate as a colorless gum (291 mg, 80% p., 51% y.). m/z [M+H]⁺=421.5.

### Intermediate 106C: 6-[phenyl(4-piperidyl)methyl]-2,3,4,5-tetrahydro-1H-1-benzazepine dihydrochloride

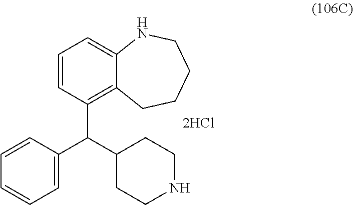 [SMILES: c1ccc(C(c2cccc3c2CCCCN3)C2CCNCC2)cc1]

To a stirred solution of tert-butyl 4-[phenyl(2,3,4,5-tetrahydro-1H-1-benzazepin-6-yl)methyl]piperidine-1-carboxylate (290 mg, 0.690 mmol) in CPME (2.1 mL) was added a 3 M solution of HCl in CPME (9.2 mL, 27.6 mmol). The reaction mixture was stirred at ambient temperature overnight. Et₂O(1.3 mL) was added. The mixture was stirred for 1 h. The resulting precipitate was filtered, washed with Et₂O, and dried under reduced pressure at ambient temperature overnight to afford 6-[phenyl(4-piperidyl)methyl]-2,3,4,5-tetrahydro-1H-1-benzazepine dihydrochloride as an off-white solid (242 mg, 80% p., 71% y.). m/z [M+H]⁺=321.5.

### Intermediate 106D: tert-butyl(4S)-5-amino-4-[6-methyl-1-oxo-5-[4-[phenyl(2,3,4,5-tetrahydro-1H-1-benzazepin-6-yl)methyl]piperidine-1-carbonyl]isoindolin-2-yl]-5-oxo-pentanoate

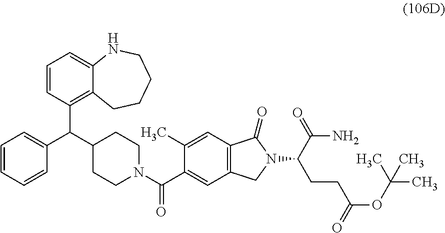 [SMILES: Cc1cc2c(cc1C(=O)N1CCC(C(c3ccccc3)c3cccc4c3CCCCN4)CC1)CN([C@@H](CCC(=O)OC(C)(C)C)C(N)=O)C2=O]

To a stirred solution of 2-[(1S)-4-tert-butoxy-1-carbamoyl-4-oxo-butyl]-6-methyl-1-oxo-isoindoline-5-carboxylic acid (253 mg, 0.671 mmol) in DCM (6.1 mL) were added successively DIPEA (533 μL, 3.05 mmol), 6-[phenyl(4-piperidyl)methyl]-2,3,4,5-tetrahydro-1H-1-benzazepine dihydrochloride (240 mg, 0.610 mmol), and then a solution of T3P in DCM (53% w/w, 514 μL, 0.915 mmol). The reaction mixture was stirred for 2 h. Additional solution of T3P in DCM (53% w/w, 171 μL, 0.305 mmol) was added and the reaction mixture was stirred for 1 h. The reaction was quenched with the addition of water. The aqueous layer was extracted with EtOAc (3×). The combined organic layers were washed with brine, dried over Na₂SO₄, filtered, and concentrated under reduced pressure. The crude was purified by flash chromatography on silica gel using a gradient of EtOAc/EtOH (3/1) in n-heptane from 1% to 100% to afford tert-butyl(4S)-5-amino-4-[6-methyl-1-oxo-5-[4-[phenyl(2,3,4,5-tetrahydro-1H-1-benzazepin-6-yl)methyl]piperidine-1-carbonyl]isoindolin-2-yl]-5-oxo-pentanoate as an off-white solid (232 mg, 99% p., 55% y.). m/z [M+H]⁺=679.5.

### Example 106

To a stirred solution of tert-butyl(4S)-5-amino-4-[6-methyl-1-oxo-5-[4-[phenyl(2,3,4,5-tetrahydro-1H-1-benzazepin-6-yl)methyl]piperidine-1-carbonyl]isoindolin-2-yl]-5-oxo-pentanoate (30.0 mg, 0.0442 mmol) in acetonitrile (440 μL) was added 4-methylbenzenesulfonic acid hydrate (84.1 mg, 0.442 mmol). The reaction mixture was stirred at 80° C. for 4 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). Acetonitrile was removed under reduced pressure. The aqueous layer was basified with a saturated aqueous solution of NaHCO₃. The precipitate was triturated for 15 min, filtered, washed with water and dried under reduced pressure at 50° C. overnight to afford (3S)-3-[6-methyl-1-oxo-5-[4-[phenyl(2,3,4,5-tetrahydro-1H-1-benzazepin-6-yl)methyl]piperidine-1-carbonyl]isoindolin-2-yl]piperidine-2,6-dione as a white solid (15.5 mg, 100% p., 58% y.). m/z [M+H]⁺=605.3. ¹H NMR (600 MHz, DMSO-d₆): δ (ppm) 10.97 (br s, 1H), 7.64-7.55 (m, 1H), 7.46-7.17 (m, 5H), 7.16-7.05 (m, 1H), 7.05-6.84 (m, 2H), 6.69-6.52 (m, 1H), 5.16-5.04 (m, 1H), 5.00-4.78 (m, 1H), 4.56-4.22 (m, 3H), 3.99-3.92 (m, 1H), 3.27-2.55 (m, 9H), 2.48-2.18 (m, 5H), 2.03-1.90 (m, 1H), 1.77-0.79 (m, 8H).

### Example 107

### (3S)-3-[6-methyl-1-oxo-5-[4-[phenyl(1,2,3,5-tetrahydro-4,1-benzoxazepin-6-yl)methyl]piperidine-1-carbonyl]isoindolin-2-yl]piperidine-2,6-dione

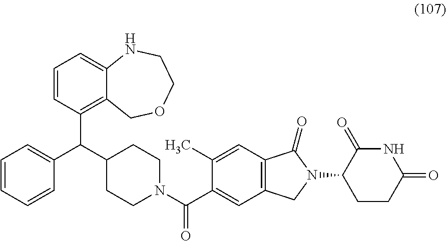 [SMILES: Cc1cc2c(cc1C(=O)N1CCC(C(c3ccccc3)c3cccc4c3COCCN4)CC1)CN([C@H]1CCC(=O)NC1=O)C2=O]

### Intermediate 107A: tert-butyl 4-[phenyl(1,2,3,5-tetrahydro-4,1-benzoxazepin-6-yl)methylene]piperidine-1-carboxylate

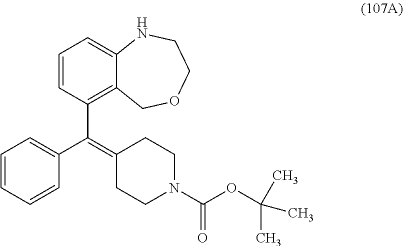 [SMILES: CC(C)(C)OC(=O)N1CCC(=C(c2ccccc2)c2cccc3c2COCCN3)CC1]

To a stirred solution of tert-butyl 4-[bromo (phenyl)methylene]piperidine-1-carboxylate (500 mg, 1.42 mmol) in CPME (9.0 mL) were added B₂pin₂(396 mg, 1.56 mmol) and Cs₂CO₃ (2.77 g, 8.52 mmol). The reaction mixture was degassed with argon for 5 min. Pd(PPh3) 4 (49.2 mg, 0.0426 mmol) was added and the reaction mixture was stirred at 80° C. for 2 h. The reaction mixture was cooled to ambient temperature. A solution of 6-bromo-1,2,3,5-tetrahydro-4,1-benzoxazepine (356 mg, 1.56 mmol) in CPME (3.0 mL) and water (3.0 mL) were added and the reaction mixture was stirred at 110° C. overnight. The reaction mixture was cooled to ambient temperature and diluted with water. The aqueous layer was extracted with EtOAc (3×). The combined organic layers were washed with brine, dried over Na₂SO₄, filtered, and concentrated under reduced pressure. The crude was purified by flash chromatography on silica gel using a gradient of EtOAc/EtOH (3/1) in n-heptane from 1% to 100% to afford tert-butyl 4-[phenyl(1,2,3,5-tetrahydro-4,1-benzoxazepin-6-yl)methylene]piperidine-1-carboxylate as an orange solid (302 mg, 31% p., 16% y.). m/z [M+Na]⁺=443.3.

### Intermediate 107B: tert-butyl 4-[phenyl(1,2,3,5-tetrahydro-4,1-benzoxazepin-6-yl)methyl]piperidine-1-carboxylate

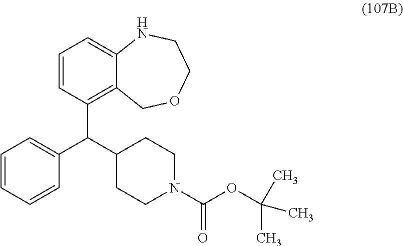 [SMILES: CC(C)(C)OC(=O)N1CCC(C(c2ccccc2)c2cccc3c2COCCN3)CC1]

In a sealed vial, a stirred solution of tert-butyl 4-[phenyl(1,2,3,5-tetrahydro-4,1-benzoxazepin-6-yl)methylene]piperidine-1-carboxylate (300 mg, 0.713 mmol) in EtOAc (4.8 mL) was degassed with argon for 5 min and palladium on carbon (380 mg, 0.357 mmol) and triethylsilane (570 μL, 3.57 mmol) were added. The mixture was stirred at 70° C. overnight and then cooled down to ambient temperature. Additional AcOH (408 μL, 7.13 mmol), palladium on carbon (759 mg, 0.713 mmol), and then triethylsilane (570 μL, 3.57 mmol) were added. The mixture was stirred at 70° C. overnight and then cooled down to ambient temperature. The suspension was filtered through a glass fiber filter and rinsed with EtOAc. The filtrate was concentrated under reduced pressure to afford tert-butyl 4-[phenyl(1,2,3,5-tetrahydro-4,1-benzoxazepin-6-yl)methyl]piperidine-1-carboxylate as a white solid (98.0 mg, 84% p., 27% y.). m/z [M+H]⁺=445.4. ¹H NMR (400 MHz, DMSO-d₆): δ (ppm) 7.35-7.27 (m, 2H), 7.24 (t, J=7.6 Hz, 2H), 7.13 (q, J=7.4 Hz, 1H), 7.07-6.97 (m, 2H), 6.73 (dd, J=7.3, 1.6 Hz, 1H), 5.48 (s, 1H), 4.83 (d, J=13.4 Hz, 1H), 4.48 (d, J=13.5 Hz, 1H), 3.99 (d, J=10.8 Hz, 1H), 3.87 (s, 2H), 3.77-3.66 (m, 1H), 3.66-3.54 (m, 1H), 3.07-2.83 (m, 2H), 2.66 (s, 2H), 2.42-2.22 (m, 1H), 1.37 (s, 11H), 1.14-0.98 (m, 1H), 0.98-0.77 (m, 1H).

### Intermediate 107C: 6-[phenyl(4-piperidyl)methyl]-1,2,3,5-tetrahydro-4,1-benzoxazepine dihydrochloride

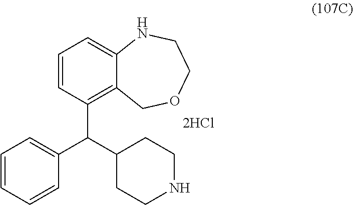 [SMILES: c1ccc(C(c2cccc3c2COCCN3)C2CCNCC2)cc1]

To a stirred solution of tert-butyl 4-[phenyl(1,2,3,5-tetrahydro-4,1-benzoxazepin-6-yl)methyl]piperidine-1-carboxylate (95.0 mg, 0.225 mmol) in CPME (690 μL) was added a 3 M solution of HCl in CPME (3.0 mL, 8.99 mmol). The reaction mixture was stirred at ambient temperature overnight. Et₂O(430 μL) was added. The mixture was stirred for 1 h. The resulting precipitate was filtered, washed with Et₂O, and dried under reduced pressure at ambient temperature overnight to afford 6-[phenyl(4-piperidyl)methyl]-1,2,3,5-tetrahydro-4,1-benzoxazepine dihydrochloride as a white solid (75.4 mg, 87% p., 74% y.). m/z [M+H]⁺=323.4.

### Intermediate 107D: tert-butyl(4S)-5-amino-4-[6-methyl-1-oxo-5-[4-[phenyl(1,2,3,5-tetrahydro-4,1-benzoxazepin-6-yl)methyl]piperidine-1-carbonyl]isoindolin-2-yl]-5-oxo-pentanoate

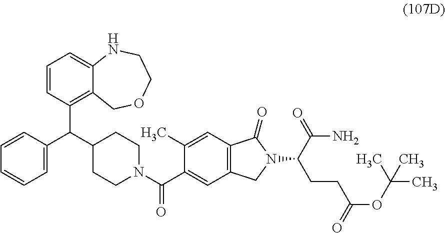 [SMILES: Cc1cc2c(cc1C(=O)N1CCC(C(c3ccccc3)c3cccc4c3COCCN4)CC1)CN([C@@H](CCC(=O)OC(C)(C)C)C(N)=O)C2=O]

To a stirred solution of 2-[(1S)-4-tert-butoxy-1-carbamoyl-4-oxo-butyl]-6-methyl-1-oxo-isoindoline-5-carboxylic acid (78.5 mg, 0.209 mmol) in DCM (1.9 mL) were added successively DIPEA (166 μL, 0.948 mmol), 6-[phenyl(4-piperidyl)methyl]-1,2,3,5-tetrahydro-4,1-benzoxazepine dihydrochloride (75.0 mg, 0.190 mmol), and then a solution of T3P in DCM (53% w/w, 160 μL, 0.285 mmol). The reaction mixture was stirred for 2 h. Additional solution of T3P in DCM (53% w/w, 53.3 μL, 0.0948 mmol) was added and the reaction mixture was stirred for 1 h. The reaction was quenched with the addition of water. The aqueous layer was extracted with EtOAc (3×). The combined organic layers were washed with brine, dried over Na₂SO₄, filtered, and concentrated under reduced pressure. The crude was purified by flash chromatography on silica using a gradient of EtOAc/EtOH (3/1) in n-heptane from 1% to 100% to afford tert-butyl(4S)-5-amino-4-[6-methyl-1-oxo-5-[4-[phenyl(1,2,3,5-tetrahydro-4,1-benzoxazepin-6-yl)methyl]piperidine-1-carbonyl]isoindolin-2-yl]-5-oxo-pentanoate as an off-white solid (92.1 mg, 86% p., 61% y.). m/z [M+H]⁺=681.5.

### Example 107

To a stirred solution of tert-butyl(4S)-5-amino-4-[6-methyl-1-oxo-5-[4-[phenyl(1,2,3,5-tetrahydro-4,1-benzoxazepin-6-yl)methyl]piperidine-1-carbonyl]isoindolin-2-yl]-5-oxo-pentanoate (90.0 mg, 0.132 mmol) in acetonitrile (1.3 mL) was added 4-methylbenzenesulfonic acid hydrate (251 mg, 1.32 mmol). The reaction mixture was stirred at 80° C. for 4 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). Acetonitrile was removed under reduced pressure. The aqueous layer was basified with a saturated aqueous solution of NaHCO₃. The precipitate was triturated for 15 min, filtered, washed with water, and dried under reduced pressure at 50° C. overnight to afford (3S)-3-[6-methyl-1-oxo-5-[4-[phenyl(1,2,3,5-tetrahydro-4,1-benzoxazepin-6-yl)methyl]piperidine-1-carbonyl]isoindolin-2-yl]piperidine-2,6-dione as a white solid (12.0 mg, 98.6% p., 15% y.). m/z [M+H]⁺=607.6. ¹H NMR (600 MHz, DMSO-d₆): δ (ppm) 10.97 (s, 1H), 7.64-7.56 (m, 1H), 7.49-7.17 (m, 5H), 7.16-6.94 (m, 3H), 6.77-6.64 (m, 1H), 5.53-5.39 (m, 1H), 5.15-5.00 (m, 1H), 4.90-4.76 (m, 1H), 4.57-4.20 (m, 4H), 4.12-3.96 (m, 1H), 3.82-3.51 (m, 2H), 3.28-3.10 (m, 1H), 3.05-2.55 (m, 6H), 2.48-2.18 (m, 5H), 2.05-1.92 (m, 1H), 1.63-0.83 (m, 4H).

### Example 108

### (3S)-3-[5-[4-[2,3-dihydrobenzofuran-4-yl(phenyl)methyl]piperidine-1-carbonyl]-6-methyl-1-oxo-isoindolin-2-yl]piperidine-2,6-dione

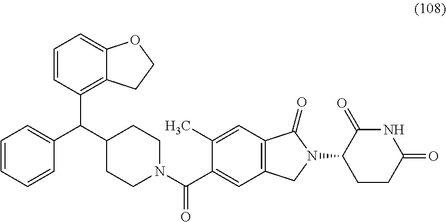 [SMILES: Cc1cc2c(cc1C(=O)N1CCC(C(c3ccccc3)c3cccc4c3CCO4)CC1)CN([C@H]1CCC(=O)NC1=O)C2=O]

### Intermediate 108A: tert-butyl 4-[benzofuran-4-yl(phenyl)methylene]piperidine-1-carboxylate

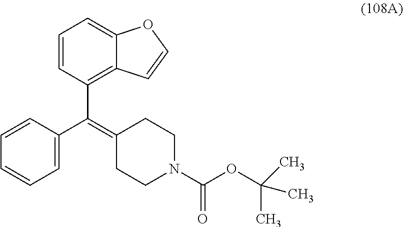 [SMILES: CC(C)(C)OC(=O)N1CCC(=C(c2ccccc2)c2cccc3occc23)CC1]

To a stirred solution of tert-butyl 4-[bromo (phenyl)methylene]piperidine-1-carboxylate (500 mg, 1.42 mmol) in CPME (9.0 mL) were added B₂pin₂(396 mg, 1.56 mmol) and Cs₂CO₃ (2.77 g, 8.52 mmol). The reaction mixture was degassed with argon for 5 min. Pd(PPh3) 4 (82.0 mg, 0.0710 mmol) was added and the reaction mixture was stirred at 80° C. for 4 h. The reaction mixture was cooled to ambient temperature. A solution of 4-bromobenzofuran (350 mg, 1.70 mmol) in CPME (3.0 mL) and water (3.0 mL) were added and the reaction mixture was stirred at 110° C. overnight. The reaction mixture was partitioned between EtOAc and water. The layers were separated and the aqueous layer was extracted with EtOAc (2×). The combined organic layers were washed with brine, dried over Na₂SO₄, filtered, and concentrated under reduced pressure. The crude was purified by flash chromatography on silica gel using a gradient of EtOAc in toluene from 0% to 10%. The desired fractions were combined, concentrated, and dried under reduced pressure to afford tert-butyl 4-[benzofuran-4-yl(phenyl)methylene]piperidine-1-carboxylate as a yellow solid (415 mg, 97% p., 73% y.). m/z [M-Boc+2H]⁺=290.3, ¹H NMR (400 MHz, DMSO-d₆): δ (ppm) 7.92 (d, J=2.2 Hz, 1H), 7.48 (d, J=8.3 Hz, 1H), 7.33-7.27 (m, 3H), 7.24-7.16 (m, 3H), 7.03 (dd, J=7.4, 0.9 Hz, 1H), 6.73 (dd, J=2.3, 1.0 Hz, 1H), 3.44 (q, J=6.4 Hz, 2H), 3.34 (t, J=5.9 Hz, 2H), 2.32 (t, J=5.8 Hz, 2H), 2.12-2.01 (m, 2H), 1.39 (d, J=1.4 Hz, 9H).

### Intermediate 108B: tert-butyl 4-[2,3-dihydrobenzofuran-4-yl(phenyl)methyl]piperidine-1-carboxylate

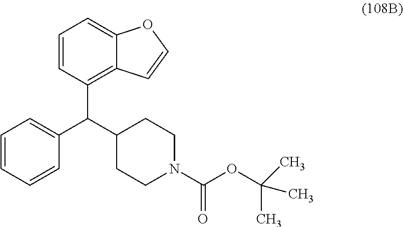 [SMILES: CC(C)(C)OC(=O)N1CCC(C(c2ccccc2)c2cccc3occc23)CC1]

In a sealed vial, to a stirred suspension of tert-butyl 4-[benzofuran-4-yl(phenyl)methylene]piperidine-1-carboxylate (413 mg, 1.06 mmol) in EtOAc (11 mL) and palladium on carbon (10% w/w, 564 mg, 0.530 mmol) under argon was added triethylsilane (686 μL, 4.24 mmol). The suspension was stirred for 3.5 h at 70° C. The reaction mixture was then filtered over fiber glass and concentrated under reduced pressure to afford tert-butyl 4-[2,3-dihydrobenzofuran-4-yl(phenyl)methyl]piperidine-1-carboxylate as a colorless oil (679 mg, 56% p., 91% y.). m/z [M-Boc+2H]⁺=294.4, 1H NMR (400 MHz, DMSO-d₆): δ (ppm) 7.39-7.34 (m, 1H), 7.35-7.30 (m, 2H), 7.26 (dd, J=8.4, 6.8 Hz, 2H), 7.19-7.13 (m, 1H), 7.06 (t, J=7.8 Hz, 1H), 6.98 (d, J=7.7 Hz, 1H), 6.57-6.50 (m, 1H), 4.54-4.39 (m, 2H), 3.88 (s, 2H), 3.61-3.53 (m, 1H), 3.29-3.20 (m, 1H), 3.04 (dt, J=16.3, 8.9 Hz, 1H), 2.68 (s, 2H), 2.43-2.29 (m, 1H), 1.52 (d, J=12.8 Hz, 1H), 1.37 (s, 9H), 1.31 (d, J=12.5 Hz, 1H), 0.97 (dt, J=10.8, 4.6 Hz, 1H).

### Intermediate 108C: 4-[2,3-dihydrobenzofuran-4-yl(phenyl)methyl]piperidine hydrochloride

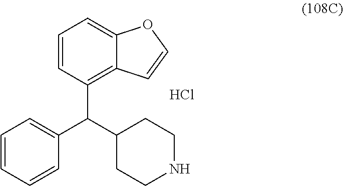 [SMILES: c1ccc(C(c2cccc3occc23)C2CCNCC2)cc1 ; Cl]

In a round-bottom flask, tert-butyl 4-[2,3-dihydrobenzofuran-4-yl(phenyl)methyl]piperidine-1-carboxylate (417 mg, 1.06 mmol) was treated with a 3 M solution of HCl in CPME (7.1 mL, 21.2 mmol). The reaction mixture was stirred at ambient temperature overnight. The suspension was diluted with Et₂O, filtered, washed with Et₂O, and dried under reduced pressure to afford 4-[2,3-dihydrobenzofuran-4-yl(phenyl)methyl]piperidine hydrochloride as a white solid (282 mg, 91% p., 73% y.). m/z [M+H]⁺=294.4, ¹H NMR (400 MHz, DMSO-d₆): δ (ppm) 8.69 (s, 1H), 8.43 (s, 1H), 7.39-7.27 (m, 4H), 7.22-7.14 (m, 1H), 7.08 (t, J=7.8 Hz, 1H), 6.97 (d, J=7.8 Hz, 1H), 6.56 (d, J=7.8 Hz, 1H), 4.48 (dt, J=9.9, 7.5 Hz, 2H), 3.56 (d, J=11.2 Hz, 1H), 3.28-3.17 (m, 3H), 3.11-3.02 (m, 1H), 2.83 (p, J=12.0 Hz, 2H), 2.54 (d, J=11.4 Hz, 1H), 1.68 (d, J=14.0 Hz, 1H), 1.47 (d, J=14.0 Hz, 1H), 1.37-1.18 (m, 2H).

### Intermediate 108D: tert-butyl(4S)-5-amino-4-[5-[4-[2,3-dihydrobenzofuran-4-yl(phenyl)methyl]piperidine-1-carbonyl]-6-methyl-1-oxo-isoindolin-2-yl]-5-oxo-pentanoate

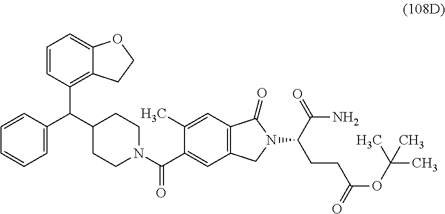 [SMILES: Cc1cc2c(cc1C(=O)N1CCC(C(c3ccccc3)c3cccc4c3CCO4)CC1)CN([C@@H](CCC(=O)OC(C)(C)C)C(N)=O)C2=O]

In a round-bottom flask, were combined 6-methyl-1-oxo-2-[(1S)-4-tert-butoxy-1-carbamoyl-4-oxo-butyl]isoindoline-5-carboxylic acid (498 mg, 1.30 mmol) and 4-[2,3-dihydrobenzofuran-4-yl(phenyl)methyl]piperidine hydrochloride (282 mg, 0.855 mmol) in dry DCM (3.5 mL). Then, DIPEA (883 μL, 5.05 mmol) was added, followed by the addition of a solution of T3P in DCM (53% w/w, 720 μL, 1.28 mmol) at 0° C. The reaction mixture was stirred at 0° C. for 1.5 h. The reaction was quenched with the addition of a saturated aqueous solution of NaHCO₃. DCM was added. The layers were separated and the aqueous layer was extracted with DCM. The combined organic layers were washed with brine, dried over Na₂SO₄, filtered, and concentrated under reduced pressure. The crude was purified by flash chromatography on silica gel using a gradient of EtOAc/EtOH (3/1) in toluene from 0% to 100% to afford tert-butyl(4S)-5-amino-4-[5-[4-[2,3-dihydrobenzofuran-4-yl(phenyl)methyl]piperidine-1-carbonyl]-6-methyl-1-oxo-isoindolin-2-yl]-5-oxo-pentanoate as an off-white foam (359 mg, 89% p., 57% y.). m/z [M+H]⁺=652.6.

### Example 108

To a stirred solution of tert-butyl(4S)-5-amino-4-[5-[4-[2,3-dihydrobenzofuran-4-yl(phenyl)methyl]piperidine-1-carbonyl]-6-methyl-1-oxo-isoindolin-2-yl]-5-oxo-pentanoate (80.0 mg, 0.123 mmol) in dry acetonitrile (890 μL) was added 4-methylbenzenesulfonic acid hydrate (93.4 mg, 0.491 mmol). The reaction mixture was stirred at 90° C. for 2 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure until evaporation of the acetonitrile. A saturated aqueous solution of NaHCO₃ was added. The resulting precipitate was filtered and dried under reduced pressure at 50° C. overnight to afford (3S)-3-[5-[4-[2,3-dihydrobenzofuran-4-yl(phenyl)methyl]piperidine-1-carbonyl]-6-methyl-1-oxo-isoindolin-2-yl]piperidine-2,6-dione as a white powder (36.1 mg, 90.4% p., 46% y.). m/z [M+H]⁺=578.3, ¹H NMR (600 MHz, DMSO-d₆): δ (ppm) 10.97 (br s, 1H), 7.65-7.57 (m, 1H), 7.46-6.91 (m, 8H), 6.57-6.48 (m, 1H), 5.09 (br dd, J=13.1, 5.2 Hz, 1H), 4.65-4.35 (m, 4H), 4.35-4.20 (m, 1H), 3.67-3.53 (m, 1H), 3.29-3.16 (m, 2H), 3.12-2.68 (m, 4H), 2.63-2.52 (m, 2H), 2.46-2.21 (m, 4H), 2.04-1.92 (m, 1H), 1.79-0.88 (m, 4H).

### Example 109

### (3S)-3-[5-[4-[1H-indazol-4-yl(phenyl)methyl]piperidine-1-carbonyl]-6-methyl-1-oxo-isoindolin-2-yl]piperidine-2,6-dione

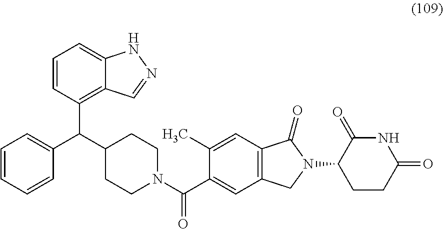 [SMILES: Cc1cc2c(cc1C(=O)N1CCC(C(c3ccccc3)c3cccc4[nH]ncc34)CC1)CN([C@H]1CCC(=O)NC1=O)C2=O]

### Intermediate 109A: tert-butyl 4-[1H-indazol-4-yl(phenyl)methylene]piperidine-1-carboxylate

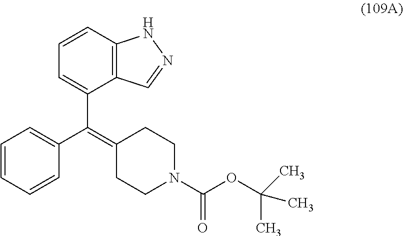 [SMILES: CC(C)(C)OC(=O)N1CCC(=C(c2ccccc2)c2cccc3[nH]ncc23)CC1]

To a stirred solution of tert-butyl 4-[bromo (phenyl)methylene]piperidine-1-carboxylate (250 mg, 0.710 mmol) and 4-(tetramethyl-1,3,2-dioxaborolan-2-yl)-1H-indazole (214 mg, 0.852 mmol) in dry DME (4.4 mL) was added Na₂CO₃ (301 mg, 2.84 mmol). The mixture was degassed with argon for 5 min and Pd₂(dba)₃ (34.2 mg, 0.0355 mmol) was added. The suspension was stirred at 100° C. overnight. The suspension was filtered over talcum powder and rinsed with EtOAc. The filtrate was concentrated under reduced pressure and water was added. The aqueous layer was extracted with EtOAc (2×). The combined organic layers were washed with brine, dried over Na₂SO₄, filtered, and concentrated under reduced pressure. The crude was purified by flash chromatography on silica gel using a gradient of EtOAc in cyclohexane from 5% to 50% to afford tert-butyl 4-[1H-indazol-4-yl(phenyl)methylene]piperidine-1-carboxylate as a light brown solid (164 mg, 75% p., 44% y.). m/z [M+H]⁺=390.4, ¹H NMR (400 MHz, DMSO-d₆): δ (ppm) 13.05 (s, 1H), 7.81 (s, 1H), 7.42 (d, J=8.5 Hz, 1H), 7.35-7.10 (m, 6H), 6.90 (d, J=7.0 Hz, 1H), 3.46 (s, 2H), 2.33 (t, J=5.8 Hz, 2H), 2.08 (t, J=5.8 Hz, 2H), 1.39 (s, 9H), 1.07 (s, 2H).

### Intermediate 109B: tert-butyl 4-[1H-indazol-4-yl(phenyl)methyl]piperidine-1-carboxylate

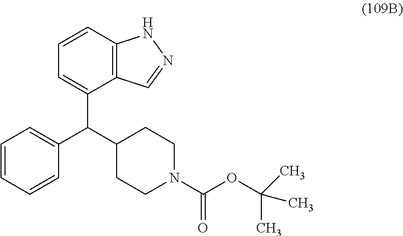 [SMILES: CC(C)(C)OC(=O)N1CCC(C(c2ccccc2)c2cccc3[nH]ncc23)CC1]

In a sealed vial, a stirred solution of tert-butyl 4-[1H-indazol-4-yl(phenyl)methylene]piperidine-1-carboxylate (160 mg, 0.308 mmol) in EtOAc (3.1 mL) was degassed with argon for 5 min. AcOH (176 μL, 3.08 mmol), palladium on carbon (10% w/w, 65.6 mg, 0.0616 mmol), and triethylsilane (246 μL, 1.54 mmol) were added. The mixture was stirred at 70° C. overnight. Additional degassed EtOH (2.0 mL), palladium on carbon (10% w/w, 65.6 mg, 0.0616 mmol), and triethylsilane (246 μL, 1.54 mmol) were added at ambient temperature and the reaction mixture was stirred at 70° C. overnight. Additional palladium on carbon (10% w/w, 197 mg, 1.85 mmol) and triethylsilane (149 μL, 0.924 mmol) were added at ambient temperature. The reaction mixture was stirred at 70° C. overnight and then cooled to ambient temperature. The suspension was filtered over glass fiber and rinsed with EtOAc and EtOH. The filtrate was concentrated under reduced pressure to afford tert-butyl 4-[1H-indazol-4-yl(phenyl)methyl]piperidine-1-carboxylate as a light brown oil (260 mg, 53% p., quant. yield)). m/z [M−tBu+H]⁺=336.4.

### Intermediate 109C: 4-[phenyl(4-piperidyl)methyl]-1H-indazole hydrochloride

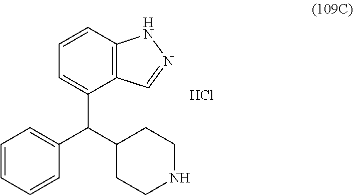 [SMILES: c1ccc(C(c2cccc3[nH]ncc23)C2CCNCC2)cc1 ; Cl]

To a stirred solution of tert-butyl 4-[1H-indazol-4-yl(phenyl)methyl]piperidine-1-carboxylate (256 mg, 0.347 mmol) in CPME (1.1 mL) was added a 3 M solution of HCl in CPME (4.6 mL, 13.9 mmol). The reaction mixture was stirred at ambient temperature overnight. Et₂O(670 μL) was added. The mixture was stirred for 1 h. The resulting precipitate was filtered, washed with Et₂O, and dried under reduced pressure at ambient temperature to afford 4-[phenyl(4-piperidyl)methyl]-1H-indazole hydrochloride as an off-white solid (75.0 mg, 95% p., 63% y.). m/z [M+H]⁺=292.4.

### Intermediate 109D: tert-butyl(4S)-5-amino-4-[5-[4-[1H-indazol-4-yl(phenyl)methyl]piperidine-1-carbonyl]-6-methyl-1-oxo-isoindolin-2-yl]-5-oxo-pentanoate

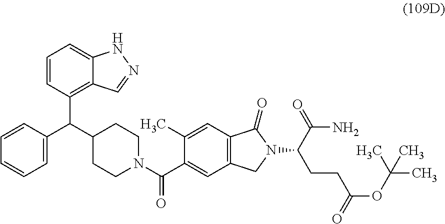 [SMILES: Cc1cc2c(cc1C(=O)N1CCC(C(c3ccccc3)c3cccc4[nH]ncc34)CC1)CN([C@@H](CCC(=O)OC(C)(C)C)C(N)=O)C2=O]

In a round-bottom flask, under nitrogen, 2-[(1S)-4-tert-butoxy-1-carbamoyl-4-oxo-butyl]-6-methyl-1-oxo-isoindoline-5-carboxylic acid (86.4 mg, 0.229 mmol) and 4-[phenyl(4-piperidyl)methyl]-1H-indazole hydrochloride (72.0 mg, 0.209 mmol) were in suspension in dry DCM (850 μL). DIPEA (146 μL, 0.835 mmol) was added, followed by the addition of a solution of T3P in DCM (53% w/w, 99.6 mg, 0.313 mmol) at ambient temperature. The reaction mixture was stirred at ambient temperature overnight. The reaction was quenched with the addition of a saturated aqueous solution of NaHCO₃. EtOAc was added. The layers were separated, and the aqueous layer was extracted with EtOAc (2×). The combined organic layers were washed with brine, dried using a phase separator cartridge, and concentrated under reduced pressure. The crude was purified by flash chromatography on silica gel using a gradient of EtOAc/EtOH (3/1) in n-heptane from 2% to 100% to afford tert-butyl(4S)-5-amino-4-[5-[4-[1H-indazol-4-yl(phenyl)methyl]piperidine-1-carbonyl]-6-methyl-1-oxo-isoindolin-2-yl]-5-oxo-pentanoate as a white solid (61.0 mg, 98% p., 44% y.). m/z [M+H]⁺=650.5.

### Example 109

To a stirred solution of tert-butyl(4S)-5-amino-4-[5-[4-[1H-indazol-4-yl(phenyl)methyl]piperidine-1-carbonyl]-6-methyl-1-oxo-isoindolin-2-yl]-5-oxo-pentanoate (58.0 mg, 0.0875 mmol) in acetonitrile (1.0 mL) at ambient temperature was added 4-methylbenzenesulfonic acid hydrate (83.2 mg, 0.437 mmol). The reaction mixture was stirred at 80° C. for 3 h. The reaction was quenched with the addition of a saturated aqueous solution of NaHCO₃. EtOAc was added. The aqueous layer was extracted with EtOAc (2×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH) to afford the expected product. The product was solubilized in a minimum of acetone and added dropwise to n-pentane. The suspension was triturated for 30 min, filtered, washed with n-pentane, and dried under reduced pressure at 50° C. for 12 h to afford (3S)-3-[5-[4-[1H-indazol-4-yl(phenyl)methyl]piperidine-1-carbonyl]-6-methyl-1-oxo-isoindolin-2-yl]piperidine-2,6-dione as a white powder (14.9 mg, 99.5% p., 29% y.). m/z [M+H]⁺=576.3, ¹H NMR (600 MHz, DMSO-d₆): δ (ppm) 13.33-12.68 (m, 1H), 10.96 (br s, 1H), 8.68-8.15 (m, 1H), 7.65-7.56 (m, 1H), 7.53-7.42 (m, 2H), 7.42-7.17 (m, 6H), 7.16-7.04 (m, 1H), 5.14-5.03 (m, 1H), 4.59-4.22 (m, 3H), 4.15 (br dd, J=16.7, 11.4 Hz, 1H), 3.29-3.13 (m, 1H), 3.09-2.94 (m, 1H), 2.94-2.67 (m, 3H), 2.64-2.54 (m, 1H), 2.43-2.20 (m, 4H), 1.98 (s, 1H), 1.71-0.92 (m, 4H).

### Example 110

### (3S)-3-[5-[4-[(2-chloro-3-methoxy-phenyl)-deuterio-phenyl-methyl]piperidine-1-carbonyl]-6-methyl-1-oxo-isoindolin-2-yl]piperidine-2,6-dione

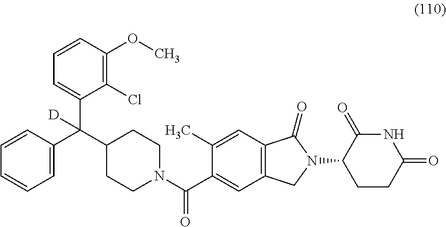 [SMILES: [2H]C(c1ccccc1)(c1cccc(OC)c1Cl)C1CCN(C(=O)c2cc3c(cc2C)C(=O)N([C@H]2CCC(=O)NC2=O)C3)CC1]

### Intermediate 110A: tert-butyl 4-[(2-chloro-3-methoxy-phenyl)-hydroxy-phenyl-methyl]piperidine-1-carboxylate

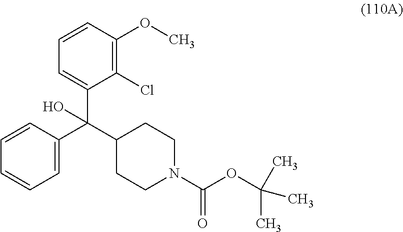 [SMILES: COc1cccc(C(O)(c2ccccc2)C2CCN(C(=O)OC(C)(C)C)CC2)c1Cl]

To a stirred solution of 1-bromo-2-chloro-3-methoxybenzene (668 mg, 2.95 mmol) in dry THF (9.4 mL) at −78° C. under argon was added a 1.6 M solution of n-butyllithium in hexanes (1.9 mL, 2.95 mmol) while maintaining the temperature below −70° C. After the end of the first addition, a solution of tert-butyl 4-benzoylpiperidine-1-carboxylate (600 mg, 1.97 mmol) in dry THF (4.7 mL) was added while maintaining the temperature below −60° C. The reaction mixture was allowed to warm up to ambient temperature overnight. The reaction was quenched with the addition of a saturated aqueous solution of NH₄Cl. Water and EtOAc were added. The layers were separated and the aqueous layer was extracted with EtOAc. The combined organic layers were washed with brine, dried using a phase separator, and concentrated under reduced pressure. The crude was purified by flash chromatography on silica gel using a gradient of EtOAc in n-heptane from 0% to 30%. The desired fractions were concentrated under reduced pressure to afford tert-butyl 4-[(2-chloro-3-methoxy-phenyl)-hydroxy-phenyl-methyl]piperidine-1-carboxylate as a white solid (570 mg, 87% p., 58% y.). m/z [M+H]⁺=432.4.

### Intermediate 110B: 4-[(2-chloro-3-methoxy-phenyl)-deuterio-phenyl-methyl]piperidine

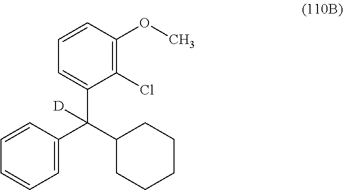 [SMILES: [2H]C(c1ccccc1)(c1cccc(OC)c1Cl)C1CCCCC1]

To a stirred solution of tert-butyl 4-[(2-chloro-3-methoxy-phenyl)-hydroxy-phenyl-methyl]piperidine-1-carboxylate (570 mg, 1.32 mmol) in DCM (9.0 mL) at −50° C. under nitrogen was added triethyl(silane-d) (650 μL, 3.96 mmol) dropwise. A solution of TFA (2.5 mL, 33.0 mmol) and water (300 μL) in DCM (4.5 mL) was added dropwise while maintaining the temperature below-40° C. The reaction mixture was stirred at −50° C. to −10° C. for 3 h and then allowed to warm up to ambient temperature overnight. The reaction mixture was slowly added to a stirred 2 M aqueous solution of NaOH (20 mL, 39.6 mmol) at 0° C. while maintaining the temperature below 10° C. The layers were separated and the aqueous layer was extracted with EtOAc (2×). The combined organic layers were washed with brine, dried using a phase separator cartridge, and concentrated under reduced pressure. The crude was purified by flash chromatography on silica gel using a gradient of EtOAc/EtOH (3/1) (+0.5% of conc. NH₄OH) in n-heptane from 0% to 100%. The desired fractions were concentrated under reduced pressure to afford 4-[(2-chloro-3-methoxy-phenyl)-deuterio-phenyl-methyl]piperidine as a white gum (308.0 mg, 22% p., 16% y.). m/z [M+H]⁺=317.4.

### Intermediate 110C: benzyl 4-[(2-chloro-3-methoxy-phenyl)-deuterio-phenyl-methyl]piperidine-1-carboxylate

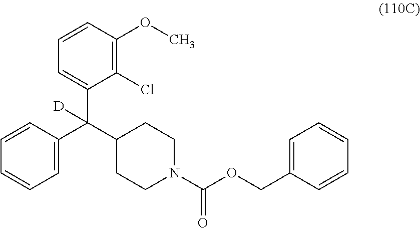 [SMILES: [2H]C(c1ccccc1)(c1cccc(OC)c1Cl)C1CCN(C(=O)OCc2ccccc2)CC1]

To a stirred solution of the mixture of 4-[(2-chloro-3-methoxy-phenyl)-deuterio-phenyl-methyl]piperidine (308 mg, 0.583 mmol) and DIPEA (204 μL, 1.17 mmol) in acetonitrile (4.2 mL), was added benzyl carbonochloridate (119 mg, 0.697 mmol). The mixture was stirred at ambient temperature for 1 h. The reaction was quenched with the addition of water. EtOAc was added. The layers were separated and the aqueous layer was extracted with EtOAc (2×). The combined organic layers were washed with brine, dried using a phase separator cartridge, and concentrated under reduced pressure. The crude was purified by flash chromatography on silica gel using a gradient of EtOAc in n-heptane from 0% to 20%. The desired fraction was concentrated under reduced pressure to afford benzyl 4-[(2-chloro-3-methoxy-phenyl)-deuterio-phenyl-methyl]piperidine-1-carboxylate as a white gum (82.0 mg, 99% p., 31% y.). m/z [M+H]⁺=451.4, ¹H NMR (400 MHz, DMSO-d₆): δ (ppm) 7.40-7.22 (m, 11H), 7.20-7.11 (m, 1H), 6.95 (dd, J=7.3, 2.3 Hz, 1H), 5.05 (s, 2H), 3.80 (s, 3H), 2.78 (s, 3H), 1.46 (d, J=13.2 Hz, 1H), 1.38 (d, J=13.1 Hz, 1H), 1.25 (s, 2H), 1.11-0.91 (m, 2H).

### Intermediate 110D: 4-[(2-chloro-3-methoxy-phenyl)-deuterio-phenyl-methyl]piperidine hydroiodide

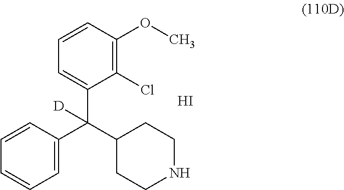

To a stirred solution of benzyl 4-[(2-chloro-3-methoxy-phenyl)-deuterio-phenyl-methyl]piperidine-1-carboxylate (79.0 mg, 0.173 mmol) in acetonitrile (1.3 mL) at ambient temperature under nitrogen was added iodo (trimethyl) silane (74.0 μL, 0.520 mmol) dropwise. The reaction mixture was stirred at ambient temperature for 1 h. The mixture was cooled to 0° C. and MeOH (843 μL, 2.08 mmol) was added dropwise. The mixture was stirred at ambient temperature for 1 h and was concentrated under reduced pressure. Et₂O was added and the resulting mixture was stirred at ambient temperature for 1 h. The solid was filtered, washed with Et₂O, and dried under reduced pressure overnight to afford 4-[(2-chloro-3-methoxy-phenyl)-deuterio-phenyl-methyl]piperidine hydroiodide as an off-white solid (66.0 mg, 100% p., 86% y.). m/z [M+H]⁺=317.4.

### Intermediate 110E: tert-butyl(4S)-5-amino-4-[5-[4-[(2-chloro-3-methoxy-phenyl)-deuterio-phenyl-methyl]piperidine-1-carbonyl]-6-methyl-1-oxo-isoindolin-2-yl]-5-oxo-pentanoate

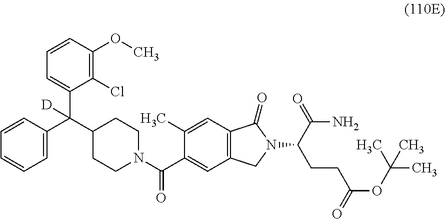 [SMILES: [2H]C(c1ccccc1)(c1cccc(OC)c1Cl)C1CCN(C(=O)c2cc3c(cc2C)C(=O)N([C@@H](CCC(=O)OC(C)(C)C)C(N)=O)C3)CC1]

In a round-bottom flask under nitrogen, 2-[(1S)-4-tert-butoxy-1-carbamoyl-4-oxo-butyl]-6-methyl-1-oxo-isoindoline-5-carboxylic acid (586 mg, 0.156 mmol) and 4-[(2-chloro-3-methoxy-phenyl)-deuterio-phenyl-methyl]piperidine hydroiodide (63.0 mg, 0.142 mmol) were in suspension in dry DCM (576 μL), DIPEA (990 μL, 0.567 mmol) was added, followed by the addition of a solution of T3P in DCM (53% w/w, 128 mg, 0.212 mmol) at ambient temperature. The reaction mixture was stirred at ambient temperature overnight. The reaction was quenched with the addition of a saturated aqueous solution of NaHCO₃. EtOAc was added. The layers were separated and the aqueous layer was extracted with EtOAc (2×). The combined organic layers were washed with brine, dried using a phase separator cartridge, and concentrated under reduced pressure. The crude was purified by flash chromatography on silica gel using a gradient of EtOAc/EtOH (3/1) in n-heptane from 2% to 100% to afford tert-butyl(4S)-5-amino-4-[5-[4-[(2-chloro-3-methoxy-phenyl)-deuterio-phenyl-methyl]piperidine-1-carbonyl]-6-methyl-1-oxo-isoindolin-2-yl]-5-oxo-pentanoate as a white solid (54.0 mg, 100% p., 56% y.). m/z [M+H]⁺=675.4.

### Example 110

To a stirred solution of tert-butyl(4S)-5-amino-4-[5-[4-[(2-chloro-3-methoxy-phenyl)-deuterio-phenyl-methyl]piperidine-1-carbonyl]-6-methyl-1-oxo-isoindolin-2-yl]-5-oxo-pentanoate (51.0 mg, 0.0755 mmol) in acetonitrile (1.0 mL) at ambient temperature was added 4-methylbenzenesulfonic acid hydrate (71.8 mg, 0.378 mmol). The reaction mixture was stirred at 80° C. for 3 h. The reaction was quenched with the addition of a saturated aqueous solution of NaHCO₃. EtOAc was added. The aqueous layer was extracted with EtOAc (2×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH) to afford the expected product. The product was solubilized in a minimum of acetone and added dropwise to n-pentane. The suspension was triturated for 30 min, filtered, washed with n-pentane, and dried under reduced pressure at 50° C. for 12 h to afford (3S)-3-[5-[4-[(2-chloro-3-methoxy-phenyl)-deuterio-phenyl-methyl]piperidine-1-carbonyl]-6-methyl-1-oxo-isoindolin-2-yl]piperidine-2,6-dione as a white powder (11.2 mg, 99.1% p., 24% y.). m/z [M+H]⁺=601.3, ¹H NMR (600 MHz, DMSO-d₆): δ (ppm) 10.97 (br s, 1H), 7.63-7.57 (m, 1H), 7.51-7.20 (m, 7H), 7.20-7.07 (m, 1H), 7.01-6.87 (m, 1H), 5.15-5.05 (m, 1H), 4.50 (br s, 1H), 4.46-4.36 (m, 1H), 4.35-4.23 (m, 1H), 3.86-3.73 (m, 3H), 3.27-3.14 (m, 1H), 3.04-2.83 (m, 2H), 2.82-2.68 (m, 1H), 2.64-2.51 (m, 2H), 2.44-2.17 (m, 4H), 2.04-1.90 (m, 1H), 1.67-1.19 (m, 2H), 1.18-0.87 (m, 2H).

### Example 111

### (3S)-3-[5-[4-[2-chloro-3-(trifluoromethoxy)phenyl]-phenyl-methyl]piperidine-1-carbonyl]-6-methyl-1-oxo-isoindolin-2-yl]piperidine-2,6-dione

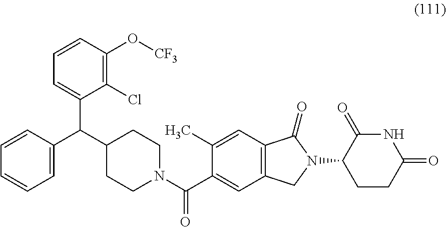 [SMILES: Cc1cc2c(cc1C(=O)N1CCC(C(c3ccccc3)c3cccc(OC(F)(F)F)c3Cl)CC1)CN([C@H]1CCC(=O)NC1=O)C2=O]

### Intermediate 111A: benzyl 4-[2-chloro-3-(trifluoromethoxy)phenyl]-hydroxy-phenyl-methyl]piperidine-1-carboxylate

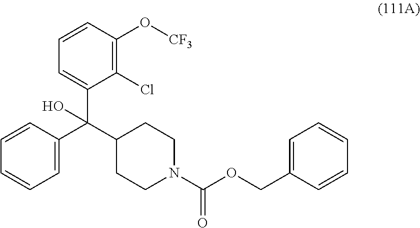 [SMILES: O=C(OCc1ccccc1)N1CCC(C(O)(c2ccccc2)c2cccc(OC(F)(F)F)c2Cl)CC1]

To a stirred solution of 1-bromo-2-chloro-3-(trifluoromethoxy)benzene (359 mg, 1.25 mmol) in dry THF (4.0 mL) at −78° C. under argon was added a 1.6 M solution of n-butyllithium in hexanes (783 μL, 1.25 mmol) while maintaining the temperature below −70° C.). Directly after the end of the first addition, a solution of benzyl 4-benzoylpiperidine-1-carboxylate (300 mg, 0.835 mmol) in dry THF (2.0 mL) was added. The reaction mixture was stirred at −70° C. for 10 min, and then at ambient temperature overnight. The reaction was quenched with the addition of a saturated aqueous solution of NH₄Cl and water. EtOAc were added. The layers were separated and the aqueous layer was extracted with EtOAc (2×). The combined organic layers were washed with brine, dried over Na₂SO₄, filtered, and concentrated under reduced pressure. The crude was purified by flash chromatography on silica gel using a gradient of EtOAc in n-heptane from 0% to 50%. The desired fractions were concentrated under reduced pressure to afford benzyl 4-[[2-chloro-3-(trifluoromethoxy)phenyl]-hydroxy-phenyl-methyl]piperidine-1-carboxylate as an off-white solid (162 mg, 47% p., 37% y.). m/z [M+H]⁺=520.4.

### Intermediate 111B: benzyl 4-[[2-chloro-3-(trifluoromethoxy)phenyl]-phenyl-methyl]piperidine-1-carboxylate

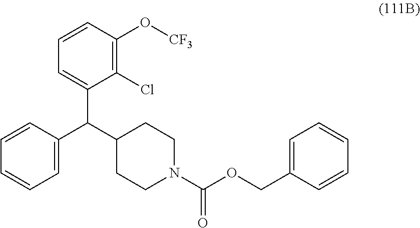 [SMILES: O=C(OCc1ccccc1)N1CCC(C(c2ccccc2)c2cccc(OC(F)(F)F)c2Cl)CC1]

To a stirred solution of triethylsilane (151 μL, 0.935 mmol) in dry DCM (4.0 mL) at −20° C. under nitrogen was added TFA (596 μL, 7.79 mmol) dropwise. A solution of benzyl 4-[[2-chloro-3-(trifluoromethoxy)phenyl]-hydroxy-phenyl-methyl]piperidine-1-carboxylate (162 mg, 0.312 mmol) in dry DCM (2.0 mL) was added dropwise. The reaction mixture was allowed to warm up to ambient temperature and stirred for 4 h. A 1 M aqueous solution of NaOH was added to the stirred reaction mixture at 0° C. until pH 12-14. The layers were separated and the aqueous layer was extracted with EtOAc (2×). The combined organic layers were washed with brine, dried over Na₂SO₄, filtered, and concentrated under reduced pressure. The crude was purified by flash chromatography on silica gel using a gradient of EtOAc in n-heptane from 0% to 30% to afford benzyl 4-[[2-chloro-3-(trifluoromethoxy)phenyl]-phenyl-methyl]piperidine-1-carboxylate as a colorless gum (54.0 mg, 73% p., 25% y.). m/z [M+H]⁺=504.4, ¹H NMR (400 MHz, DMSO-d₆): δ (ppm) 7.79 (dd, J=8.0, 1.5 Hz, 1H), 7.49 (t, J=8.1 Hz, 1H), 7.42-7.26 (m, 10H), 7.22-7.15 (m, 1H), 5.05 (s, 2H), 4.18 (d, J=11.2 Hz, 1H), 4.02-3.93 (m, 2H), 2.78 (s, 2H), 1.46-1.33 (m, 2H), 1.30-1.22 (m, 1H), 1.13-0.92 (m, 2H).

### Intermediate 111C: 4-[[2-chloro-3-(trifluoromethoxy)phenyl]-phenyl-methyl]piperidine hydroiodide

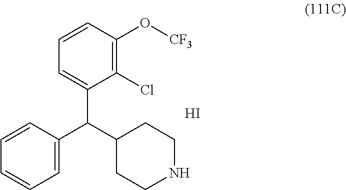

To a stirred solution of benzyl 4-[[2-chloro-3-(trifluoromethoxy)phenyl]-phenyl-methyl]piperidine-1-carboxylate (54.0 mg, 0.0782 mmol) in acetonitrile (1.0 mL) at ambient temperature under nitrogen was added iodo (trimethyl) silane (33.4 μL, 0.235 mmol). The reaction mixture was stirred at ambient temperature for 1 h. The mixture was cooled to 0° C. and MeOH (80 μL, 0.939 mmol) was added dropwise. The mixture was stirred at ambient temperature for 1 h and was concentrated under reduced pressure. Et₂O was added and the resulting mixture was stirred at ambient temperature for 1 h. The solid was filtered, washed with Et₂O, and dried under reduced pressure overnight to afford 4-[[2-chloro-3-(trifluoromethoxy)phenyl]-phenyl-methyl]piperidine hydroiodide as a yellow solid (29.0 mg, 90% p., 67% y.). m/z [M+H]⁺=370.4, ¹H NMR (400 MHz, DMSO-d₆): δ (ppm) 8.40 (s, 1H), 8.11 (s, 1H), 7.74 (d, J=7.9 Hz, 1H), 7.51 (t, J=8.0 Hz, 1H), 7.46-7.29 (m, 5H), 7.22 (t, J=7.3 Hz, 1H), 4.18 (d, J=11.1 Hz, 1H), 3.31 (s, 2H), 2.83 (s, 2H), 2.64 (t, J=11.9 Hz, 1H), 1.53 (s, 2H), 1.37-1.18 (m, 2H).

### Intermediate 111D: tert-butyl(4S)-5-amino-4-[5-[4-[2-chloro-3-(trifluoromethoxy)phenyl]-phenyl-methyl]piperidine-1-carbonyl]-6-methyl-1-oxo-isoindolin-2-yl]-5-oxo-pentanoate

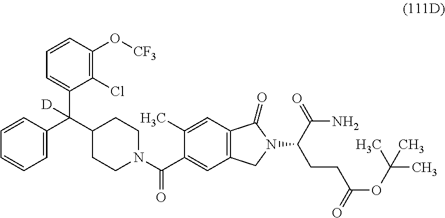 [SMILES: [2H]C(c1ccccc1)(c1cccc(OC(F)(F)F)c1Cl)C1CCN(C(=O)c2cc3c(cc2C)C(=O)N([C@@H](CCC(=O)OC(C)(C)C)C(N)=O)C3)CC1]

To a stirred solution of 2-[(1S)-4-tert-butoxy-1-carbamoyl-4-oxo-butyl]-6-methyl-1-oxo-isoindoline-5-carboxylic acid (23.7 mg, 0.0629 mmol) in DMF (500 μL) at ambient temperature were added 4-[[2-chloro-3-(trifluoromethoxy)phenyl]-phenyl-methyl]piperidine hydroiodide (29.0 mg, 0.0524 mmol), DIPEA (275 μL, 0.157 mmol), and HATU (24.7 mg, 0.0629 mmol). The reaction mixture was stirred at ambient temperature for 16 h. The reaction was quenched with the addition of a saturated aqueous solution of NH₄C1. EtOAc was added. The aqueous layer was extracted with EtOAc (2×).

The combined organic layers were washed with brine, dried over Na₂SO₄, filtered, and concentrated under reduced pressure. The crude was purified by flash chromatography on silica gel using a gradient of EtOAc/EtOH (3/1) in n-heptane from 10% to 100% to afford tert-butyl(4S)-5-amino-4-[5-[4-[2-chloro-3-(trifluoromethoxy)phenyl]-phenyl-methyl]piperidine-1-carbonyl]-6-methyl-1-oxo-isoindolin-2-yl]-5-oxo-pentanoate as an off-white solid (37.0 mg, 86% p., 83% y.). m/z [M+H]⁺=728.8.

### Example 111

To a stirred solution of tert-butyl(4S)-5-amino-4-[5-[4-[[2-chloro-3-(trifluoromethoxy)phenyl]-phenyl-methyl]piperidine-1-carbonyl]-6-methyl-1-oxo-isoindolin-2-yl]-5-oxo-pentanoate (37.0 mg, 0.0437 mmol) in acetonitrile (500 μL) at ambient temperature was added4-methylbenzenesulfonic acid hydrate (41.6 mg, 0.218 mmol). The reaction mixture was stirred at 80° C. for 2 h. The reaction was quenched with the addition of a saturated aqueous solution of NaHCO₃. EtOAc was added. The aqueous layer was extracted with EtOAc (2×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The product was solubilized in a minimum of acetone and added dropwise to a stirred solution of n-pentane. The resulting precipitate was filtered, washed with n-pentane, and dried under reduced pressure at 50° C. for 12 h to afford (3S)-3-[5-[4-[2-chloro-3-(trifluoromethoxy)phenyl]-phenyl-methyl]piperidine-1-carbonyl]-6-methyl-1-oxo-isoindolin-2-yl]piperidine-2,6-dione as a white powder (5.5 mg, 100% p., 19% y.). m/z [M+H]⁺=654.4, ¹H NMR (600 MHz, DMSO-d₆): δ (ppm) 11.26-10.45 (m, 1H), 7.86-7.73 (m, 1H), 7.63-7.57 (m, 1H), 7.54-7.24 (m, 7H), 7.23-7.12 (m, 1H), 5.16-4.94 (m, 1H), 4.58-4.16 (m, 4H), 3.30-2.52 (m, 6H), 2.42-2.20 (m, 4H), 2.04-1.87 (m, 1H), 1.57-0.97 (m, 4H).

### Example 112

### (3S)-3-[rel-(1S)-6-[4-[(R)-(2-chlorophenyl)-phenyl-methyl]piperidine-1-carbonyl]-1,5-dimethyl-3-oxo-isoindolin-2-yl]piperidine-2,6-dione

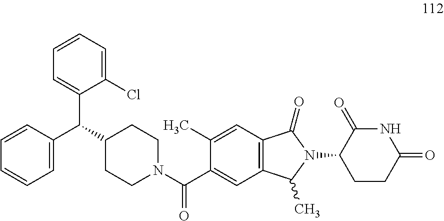 [SMILES: Cc1cc2c(cc1C(=O)N1CCC([C@H](c3ccccc3)c3ccccc3Cl)CC1)C(C)N([C@H]1CCC(=O)NC1=O)C2=O]

### Intermediate 112A: 4-bromo-2-ethyl-5-methyl-benzoic acid

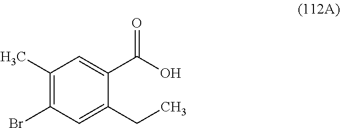

To a stirred solution of 4-bromo-2-fluoro-5-methylbenzoic acid (5.00 g, 21.5 mmol) in dry THF (86 mL) at −78° C. under nitrogen was added a solution of 3.4 M bromo (ethyl) magnesium in MeTHF (19 mL, 64.4 mmol) dropwise (T<70° C.). The reaction mixture was stirred at −78° C. for 1 h and then at ambient temperature overnight. The reaction was quenched with the addition of a 2 M aqueous solution of NaOH (100 mL). The reaction mixture was filtered through a pad of celite. The aqueous layer was extracted with EtOAc (2×). The aqueous layer was acidified with a concentrated aqueous solution of HCl (37% w/w) until pH 1. The aqueous layer was extracted with EtOAc (3×).

The combined organic layers were washed with brine, dried over Na₂SO₄, filtered, and concentrated under reduced pressure. The crude was purified by flash chromatography on silica gel using a gradient of EtOAc in cyclohexane from 1% to 20% to afford 4-bromo-2-ethyl-5-methyl-benzoic acid as a colorless liquid (1.17 g, 46% p., 10% y.). m/z [M+H]⁺=no ionization, 1H NMR (400 MHz, DMSO-d₆): δ (ppm) 12.84 (s, 1H), 7.73 (d, J=0.8 Hz, 1H), 7.54 (s, 1H), 2.87 (q, J=7.5 Hz, 2H), 2.33 (s, 3H), 1.18-1.08 (m, 3H).

### Intermediate 112B: methyl 4-bromo-2-ethyl-5-methyl-benzoate

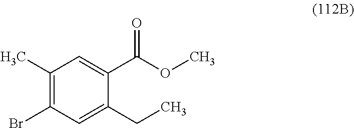

To a stirred solution of 4-bromo-2-ethyl-5-methyl-benzoic acid (1.17 g, 2.21 mmol) in DMF (5.5 mL) were added successively Cs₂CO₃ (1.44 g, 4.43 mmol) and then iodomethane (276 μL, 4.43 mmol). The reaction mixture was stirred at ambient temperature for 4 h. The reaction mixture was diluted with water. The aqueous layer was extracted with EtOAc (3×). The combined organic layers were washed with brine, dried over Na₂SO₄, filtered, and concentrated under reduced pressure. The crude was purified by flash chromatography on silica using a gradient of EtOAc in n-heptane from 1% to 20% to afford methyl 4-bromo-2-ethyl-5-methyl-benzoate as a colorless liquid (1.04 g, 50% p., 92% y.). m/z [M+H]⁺=no ionization, 1H NMR (400 MHz, DMSO-d₆): δ (ppm) 7.72 (s, 1H), 7.57 (s, 1H), 3.82 (s, 3H), 2.84 (q, J=7.5 Hz, 2H), 2.34 (s, 3H), 1.12 (t, J=7.5 Hz, 3H).

### Intermediate 112C: methyl 4-bromo-2-(1-bromoethyl)-5-methyl-benzoate

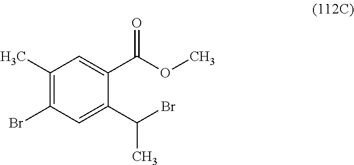

To a stirred solution of methyl 4-bromo-2-ethyl-5-methyl-benzoate (1.04 g, 4.04 mmol) in acetonitrile (20 mL) were added 1-bromopyrrolidine-2,5-dione (792 mg, 4.45 mmol) and AIBN (66.4 mg, 0.404 mmol). The reaction mixture was irradiated with a white lamp (6200 K) overnight. The mixture was concentrated under reduced pressure. The crude was purified by flash chromatography on silica gel using a gradient of EtOAc in cyclohexane from 2% to 100% to afford methyl 4-bromo-2-(1-bromoethyl)-5-methyl-benzoate as a colorless oil (971 mg, 2.05 mmol, 71% p., 51% y.). m/z [M+H]=336.0, ¹H NMR (400 MHz, DMSO-d₆): δ (ppm) 7.98 (s, 1H), 7.75 (d, J=0.8 Hz, 1H), 6.16 (q, J=6.9 Hz, 1H), 3.86 (s, 3H), 2.38 (s, 3H), 2.00 (d, J=6.9 Hz, 3H).

### Intermediate 112E: tert-butyl(4S)-5-amino-4-[rel-(1S)-6-bromo-1,5-dimethyl-3-oxo-isoindolin-2-yl]-5-oxo-pentanoate

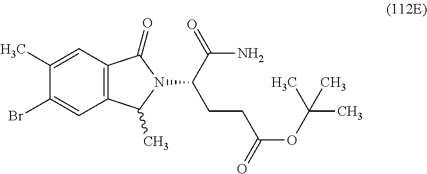 [SMILES: Cc1cc2c(cc1Br)C(C)N([C@@H](CCC(=O)OC(C)(C)C)C(N)=O)C2=O]

To a stirred suspension of tert-butyl(4S)-4-amino-4-carbamoylbutanoate hydrochloride (773 mg, 3.18 mmol) in acetonitrile (8.0 mL) was added DIPEA (1.2 mL, 6.64 mmol). The reaction mixture was stirred for 15 min and then a solution of methyl 4-bromo-2-(1-bromoethyl)-5-methyl-benzoate (970 mg, 2.89 mmol) in acetonitrile (4.5 mL) was added. The reaction mixture was stirred at ambient temperature for 1 h and then at 80° C. for 24 h. The reaction mixture was cooled to ambient temperature. The reaction was quenched with the addition of a saturated aqueous solution of NH₄Cl. The aqueous layer was extracted with EtOAc (3×). The combined organic layers were washed with brine, dried over Na₂SO₄, filtered, and concentrated under reduced pressure. The crude was purified by flash chromatography on silica using a gradient of EtOAc/EtOH (3/1) in n-heptane from 1% to 100% and then by reverse-phase preparative chromatography using a gradient of acetonitrile in water from 10% to 100% (0.1% AcOH) to afford the expected diastereomeric mixture tert-butyl(4S)-5-amino-4-(6-bromo-1,5-dimethyl-3-oxo-isoindolin-2-yl)-5-oxo-pentanoate (335 mg, 0.615 mmol, 78% p., 21% y.) as an off-white foam. Tert-butyl(4S)-5-amino-4-(6-bromo-1,5-dimethyl-3-oxo-isoindolin-2-yl)-5-oxo-pentanoate was purified by chiral supercritical chromatography (Waters Prep SFC80, Chiralpak AD-H (5 μm, 250×20 mm), CO₂/iPrOH 80/20 as mobile phase, flow rate 50 mL/min, 100 bars) to afford (second isomer eluted) as a white solid (tert-butyl(4S)-5-amino-4-[rel-(1S)-6-bromo-1,5-dimethyl-3-oxo-isoindolin-2-yl]-5-oxo-pentanoate (112 mg, 0.260 mmol, 99% p., 9% y.). m/z [M+H]⁺=425.3, ¹H NMR (400 MHz, DMSO-d₆): δ (ppm) 7.91 (s, 1H), 7.66 (s, 1H), 7.44 (s, 1H), 7.20 (s, 1H), 4.67 (q, J=6.6 Hz, 1H), 4.46 (dd, J=9.1, 5.4 Hz, 1H), 2.43 (s, 3H), 2.32-2.02 (m, 4H), 1.47 (d, J=6.7 Hz, 3H), 1.38 (s, 9H).

### Intermediate 112F: rel-(3S)-2-[(1S)-4-tert-butoxy-1-carbamoyl-4-oxo-butyl]-3,6-dimethyl-1-oxo-isoindoline-5-carboxylic acid

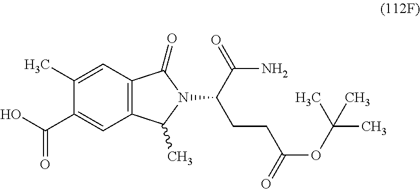 [SMILES: Cc1cc2c(cc1C(=O)O)C(C)N([C@@H](CCC(=O)OC(C)(C)C)C(N)=O)C2=O]

In a sealed vial, to a stirred solution of tert-butyl(4S)-5-amino-4-[rel-(1S)-6-bromo-1,5-dimethyl-3-oxo-isoindolin-2-yl]-5-oxo-pentanoate (110 mg, 0.259 mmol) in butyronitrile (2.6 mL) were added successively ethanedioic acid dihydrate (48.9 mg, 0.388 mmol), XantPhos (7.5 mg, 0.0129 mmol), and Pd(OAc)₂(2.9 mg, 0.0129 mmol). The reaction mixture was degassed with argon for 5 min and then acetic anhydride (35.9 μL, 0.388 mmol) and DIPEA (67.8 μL, 0.388 mmol) were added. The reaction mixture was stirred at 75° C. for 18 h. The reaction mixture was cooled to ambient temperature. The organic layer was washed with a saturated aqueous solution of NaHCO₃ (3×). The combined aqueous layers were acidified to pH 2-3 with a concentrated aqueous solution of HCl (37% w/w). The acidic aqueous layer was extracted with EtOAc (3×). The combined organic layers were washed with brine, dried over Na₂SO₄, filtered, and concentrated under reduced pressure to afford rel-(3S)-2-[(1S)-4-tert-butoxy-1-carbamoyl-4-oxo-butyl]-3,6-dimethyl-1-oxo-isoindoline-5-carboxylic acid (92.0 mg, 0.233 mmol, 99% p., 90% y.). m/z [M+H]⁺=413.4, ¹H NMR (400 MHz, DMSO-d₆): δ (ppm) 13.17 (s, 1H), 7.96 (s, 1H), 7.59 (t, J=0.8 Hz, 1H), 7.45 (s, 1H), 7.21 (s, 1H), 4.70 (q, J=6.6 Hz, 1H), 4.48 (dd, J=8.8, 5.3 Hz, 1H), 2.58 (s, 3H), 2.30-2.04 (m, 4H), 1.48 (d, J=6.6 Hz, 3H), 1.37 (s, 9H).

### Intermediate 112G: tert-butyl(4S)-5-amino-4-[rel-(1S)-6-[4-[(R)-(2-chlorophenyl)-phenyl-methyl]piperidine-1-carbonyl]-1,5-dimethyl-3-oxo-isoindolin-2-yl]-5-oxo-pentanoate

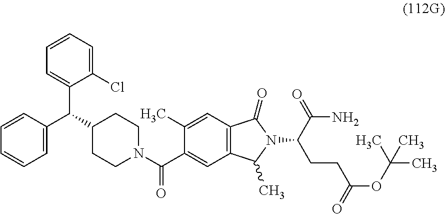 [SMILES: Cc1cc2c(cc1C(=O)N1CCC([C@H](c3ccccc3)c3ccccc3Cl)CC1)C(C)N([C@@H](CCC(=O)OC(C)(C)C)C(N)=O)C2=O]

To a stirred solution of rel-(3S)-2-[(1S)-4-tert-butoxy-1-carbamoyl-4-oxo-butyl]-3,6-dimethyl-1-oxo-isoindoline-5-carboxylic acid (90.0 mg, 0.231 mmol) in DMF (2.3 mL) were added successively DIPEA (201 μL, 1.15 mmol), 4-[(R)-(2-chlorophenyl)-phenyl-methyl]piperidine hydroiodide (105 mg, 0.254 mmol) and then HATU (58.4 μL, 0.231 mmol). The reaction mixture was stirred at ambient temperature for 2 h. The reaction was quenched with the addition of water. The aqueous layer was extracted with EtOAc (3×). The combined organic layers were washed with brine, dried over Na₂SO₄, filtered, and concentrated under reduced pressure. The crude was purified by flash chromatography on silica using a gradient of EtOAc/EtOH (3/1) in n-heptane from 1% to 100% to afford tert-butyl(4S)-5-amino-4-[rel-(1S)-6-[4-[(R)-(2-chlorophenyl)-phenyl-methyl]piperidine-1-carbonyl]-1,5-dimethyl-3-oxo-isoindolin-2-yl]-5-oxo-pentanoate as an off-white foam (156 mg, 100% p., quant. yield)). m/z [M+H]⁺=658.5.

### Example 112

To a stirred solution of tert-butyl(4S)-5-amino-4-[rel-(1S)-6-[4-[(R)-(2-chlorophenyl)-phenyl-methyl]piperidine-1-carbonyl]-1,5-dimethyl-3-oxo-isoindolin-2-yl]-5-oxo-pentanoate (155 mg, 0.235 mmol) in acetonitrile (2.4 mL) was added 4-methylbenzenesulfonic acid hydrate (224 mg, 1.18 mmol). The reaction mixture was stirred at 90° C. for 3 h. The reaction mixture was cooled to ambient temperature. The homogenous solution was added dropwise to a stirred aqueous solution of NaHCO₃. The precipitate was triturated for 15 min, filtered, washed with water, and dried under reduced pressure at 50° C. overnight to afford (3S)-3-[rel-(1S)-6-[4-[(R)-(2-chlorophenyl)-phenyl-methyl]piperidine-1-carbonyl]-1,5-dimethyl-3-oxo-isoindolin-2-yl]piperidine-2,6-dione as a white solid (79.2 mg, 92% p., 53% y.). The compound was solubilized in EtOAc. The organic layer was washed with a saturated aqueous solution of NaHCO₃ (2×), brine, dried over Na₂SO₄, filtered, and concentrated under reduced pressure. The resulting solid was solubilized in the minimum amount of DCM and the homogenous solution was added dropwise to a stirred solution of n-pentane. The mixture was stirred for 15 min. The resulting precipitate was filtered, washed with n-pentane, and dried under reduced pressure at 50° C. overnight to afford (3S)-3-[rel-(1S)-6-[4-[(R)-(2-chlorophenyl)-phenyl-methyl]piperidine-1-carbonyl]-1,5-dimethyl-3-oxo-isoindolin-2-yl]piperidine-2,6-dione as a white solid (51.9 mg, 98% p., 37% y.). m/z [M+H]⁺=584.3, ¹H NMR (600 MHz, DMSO-d₆): δ (ppm) 10.98-10.86 (m, 1H), 7.74 (br d, J=7.9 Hz, 1H), 7.57-7.41 (m, 2H), 7.40-7.36 (m, 2H), 7.34 (s, 2H), 7.31-7.23 (m, 2H), 7.22-7.17 (m, 1H), 7.13 (s, 1H), 4.72 (s, 2H), 4.51 (br d, J=12.9 Hz, 1H), 4.21-4.09 (m, 1H), 3.27-3.12 (m, 1H), 2.97 (br d, J=13.5 Hz, 1H), 2.84-2.66 (m, 2H), 2.64-2.51 (m, 3H), 2.36-2.18 (m, 3H), 1.98 (s, 1H), 1.65-1.45 (m, 1H), 1.45-1.38 (m, 3H), 1.38-1.20 (m, 1H), 1.19-1.10 (m, 1H), 1.10-0.87 (m, 1H), [α]_{D}=−38°·mL·dm⁻¹ g⁻¹ (20° C., Na lamp, 589 nm, C=1.9 mg/mL, DMSO).

### Example 113

### (3S)-3-[5-[4-[rel-(S)-(2-chloro-4-fluoro-3-methoxy-phenyl)-phenyl-methyl]piperidine-1-carbonyl]-6-methyl-1-oxo-isoindolin-2-yl]piperidine-2,6-dione

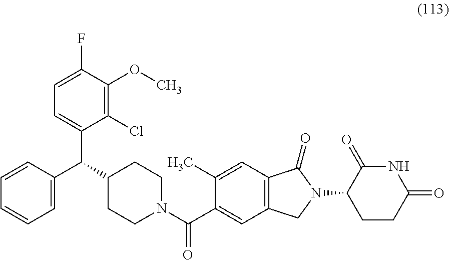 [SMILES: COc1c(F)ccc([C@@H](c2ccccc2)C2CCN(C(=O)c3cc4c(cc3C)C(=O)N([C@H]3CCC(=O)NC3=O)C4)CC2)c1Cl]

### Intermediate 113A: benzyl 4-[(2-chloro-4-fluoro-3-methoxy-phenyl)-hydroxy-phenyl-methyl]piperidine-1-carboxylate

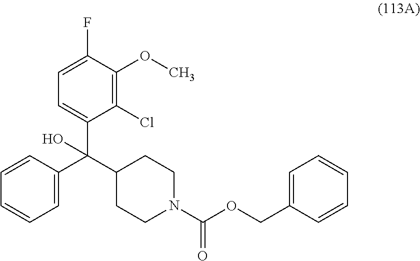 [SMILES: COc1c(F)ccc(C(O)(c2ccccc2)C2CCN(C(=O)OCc3ccccc3)CC2)c1Cl]

To a stirred solution of 1-bromo-2-chloro-4-fluoro-3-methoxybenzene (1.00 g, 3.97 mmol) in dry THF (12 mL) at −78° C. (acetone/dry ice) under argon was added over 2 min a 1.6 M solution of n-butyllithium in hexanes (2.5 mL, 3.97 mmol). After the end of the first addition, a solution of benzyl 4-benzoylpiperidine-1-carboxylate (900 mg, 2.64 mmol) in dry THF (6.0 mL) was then added. The reaction mixture was stirred at −78° C. for 1 h. The reaction was quenched with the addition of a saturated aqueous solution of NH₄Cl. Water and EtOAc were added. The layers were separated and the aqueous layer was extracted with EtOAc (2×). The combined organic layers were washed with brine, dried over Na₂SO₄, filtered, and concentrated under reduced pressure. The crude was purified by flash chromatography on silica gel using a gradient of EtOAc in n-heptane from 0% to 50%. The desired fractions were combined, concentrated, and dried under reduced pressure to afford benzyl 4-[(2-chloro-4-fluoro-3-methoxy-phenyl)-hydroxy-phenyl-methyl]piperidine-1-carboxylate as a white foam (800 mg, 61% p., 38% y.). m/z [M+H]⁺=484.3.

### Intermediate 113B: benzyl 4-[rel-(S)-(2-chloro-4-fluoro-3-methoxy-phenyl)-phenyl-methyl]piperidine-1-carboxylate

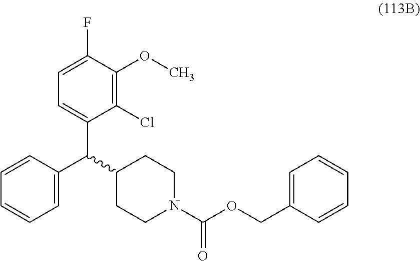 [SMILES: COc1c(F)ccc(C(c2ccccc2)C2CCN(C(=O)OCc3ccccc3)CC2)c1Cl]

In a round-bottom flask, triethylsilane (800 μL, 4.91 mmol) was solubilized in DCM (3.2 mL) at ambient temperature and TFA (3.1 mL, 40.5 mmol) was added, followed by the addition of water (300 μL). The reaction mixture was kept at −25° C. and a solution of benzyl 4-[(2-chloro-4-fluoro-3-methoxy-phenyl)-hydroxy-phenyl-methyl]piperidine-1-carboxylate (799 mg, 1.01 mmol) in DCM (2.8 mL) was added dropwise. The temperature was allowed to warm up to 5° C. over 3 h. The reaction was quenched with the addition of a saturated aqueous solution of NaHCO₃. The mixture was diluted in EtOAc. The layers were separated and the organic layer was washed with brine. The organic layer was dried over Na₂SO₄, filtered, and concentrated under reduced pressure. The crude was purified by flash chromatography on silica gel using a gradient of EtOAc in n-heptane from 0% to 50%. The desired fractions were combined, concentrated, and dried under reduced pressure to afford the racemate benzyl 4-[(2-chloro-4-fluoro-3-methoxy-phenyl)-phenyl-methyl]piperidine-1-carboxylate (326 mg, 94% p., 65% y.) as a yellow gum. Benzyl 4-[(2-chloro-4-fluoro-3-methoxy-phenyl)-phenyl-methyl]piperidine-1-carboxylate was purified by chiral supercritical chromatography (Waters Prep SFC80, Chiralpak IA (5 μm, 250×21 mm), CO₂/iPrOH 80/20 as mobile phase, flow rate 50 mL/min, 100 bars) to afford benzyl 4-[rel-(S)-(2-chloro-4-fluoro-3-methoxy-phenyl)-phenyl-methyl]piperidine-1-carboxylate (second isomer eluted) as a colorless gum (370 mg, 93% p., 19% y., e.c.=96.6%). m/z [M+H]⁺=468.3, ¹H NMR (400 MHz, DMSO-d₆): δ (ppm) 7.47 (dd, J=8.9, 5.5 Hz, 1H), 7.41-7.24 (m, 9H), 7.21-7.14 (m, 1H), 5.05 (s, 2H), 4.08 (d, J=11.2 Hz, 1H), 3.96 (d, J=13.2 Hz, 2H), 3.83 (d, J=1.2 Hz, 3H), 2.72 (d, J=38.6 Hz, 2H), 2.45 (t, J=11.2 Hz, 1H), 1.40 (dd, J=43.4, 13.2 Hz, 2H), 1.00 (dqd, J=24.6, 12.4, 4.2 Hz, 2H).

### Intermediate 113C: 4-[rel-(S)-(2-chloro-4-fluoro-3-methoxy-phenyl)-phenyl-methyl]piperidine hydroiodide

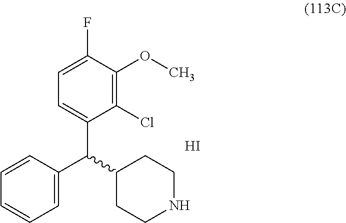

To a stirred solution of benzyl 4-[rel-(S)-(2-chloro-4-fluoro-3-methoxy-phenyl)-phenyl-methyl]piperidine-1-carboxylate (125 mg, 0.267 mmol) in dry acetonitrile (2.7 mL) at ambient temperature under nitrogen was added iodo (trimethyl) silane (114 μL, 0.801 mmol) dropwise. The reaction mixture was stirred at ambient temperature for 45 min. MeOH (130 μL, 3.21 mmol) was added dropwise. The mixture was stirred at ambient temperature for 30 min and was concentrated under reduced pressure. Et₂O was added and the resulting mixture was stirred at ambient temperature for 2 h. The solid was filtered, washed with Et₂O, and dried under reduced pressure to afford 4-[rel-(S)-(2-chloro-4-fluoro-3-methoxy-phenyl)-phenyl-methyl]piperidine hydroiodide as a beige powder (103 mg, 98% p., 82% y.). m/z [M+H]⁺=334.3, ¹H NMR (400 MHz, DMSO-d₆): δ (ppm) 8.22 (s, 2H), 7.42 (dd, J=8.9, 5.5 Hz, 1H), 7.37-7.28 (m, 5H), 7.25-7.16 (m, 1H), 4.08 (d, J=11.3 Hz, 1H), 3.84 (d, J=1.2 Hz, 3H), 3.28-3.20 (m, 2H), 2.81 (dt, J=12.9, 10.1 Hz, 2H), 2.57 (t, J=11.3 Hz, 1H), 1.53 (dd, J=30.4, 14.0 Hz, 2H), 1.24 (dt, J=27.2, 12.7 Hz, 2H).

### Intermediate 113D: tert-butyl(4S)-5-amino-4-[5-[4-rel-[(S)-(2-chloro-4-fluoro-3-methoxy-phenyl)-phenyl-methyl]piperidine-1-carbonyl]-6-methyl-1-oxo-isoindolin-2-yl]-5-oxo-pentanoate

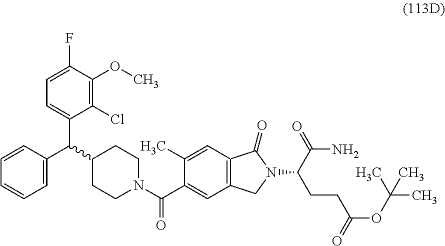 [SMILES: COc1c(F)ccc(C(c2ccccc2)C2CCN(C(=O)c3cc4c(cc3C)C(=O)N([C@@H](CCC(=O)OC(C)(C)C)C(N)=O)C4)CC2)c1Cl]

In a round-bottom flask, were combined 6-methyl-1-oxo-2-[(1S)-4-tert-butoxy-1-carbamoyl-4-oxo-butyl]isoindoline-5-carboxylic acid (151 mg, 0.394 mmol) and 4-[rel-(S)-(2-chloro-4-fluoro-3-methoxy-phenyl)-phenyl-methyl]piperidine hydroiodide (120 mg, 0.260 mmol) in DCM (1.1 mL) at 0° C. Then, DIPEA (268 μL, 1.54 mmol) was added, followed by the addition of a solution of T3P 53% in DCM (219 μL, 0.390 mmol). The reaction mixture was stirred at 0° C. for 1 h. The reaction was quenched with the addition of a saturated aqueous solution of NaHCO₃. DCM was added. The layers were separated and the aqueous layer was extracted with EtOAc (2×). The combined organic layers were washed with brine, dried over Na₂SO₄, filtered, and concentrated under reduced pressure to afford tert-butyl(4S)-5-amino-4-[5-[4-[rel-(S)-(2-chloro-4-fluoro-3-methoxy-phenyl)-phenyl-methyl]piperidine-1-carbonyl]-6-methyl-1-oxo-isoindolin-2-yl]-5-oxo-pentanoate as a white solid (99.7 mg, 100% p., 55% y.). m/z [M+H]⁺=692.5, ¹H NMR (400 MHz, DMSO-d₆): δ (ppm) 7.67-7.02 (m, 11H), 4.71 (d, J=10.1 Hz, 1H), 4.54 (d, J=17.4 Hz, 2H), 4.43 (s, 1H), 4.17-4.08 (m, 1H), 3.87-3.78 (m, 3H), 3.24 (d, J=17.9 Hz, 1H), 3.05-2.88 (m, 1H), 2.81-2.66 (m, 1H), 2.56 (d, J=10.8 Hz, 1H), 2.23 (t, J=36.5 Hz, 6H), 1.98 (d, J=9.5 Hz, 1H), 1.59 (s, 1H), 1.41-1.02 (m, 12H), [α]_{D}=−15°·mL·dm⁻¹·g⁻¹[20° C., Na lamp, 589 nm, C=1.2 mg/mL, EtOH].

### Example 113

To a stirred solution of tert-butyl(4S)-5-amino-4-[5-[4-[rel-(S)-(2-chloro-4-fluoro-3-methoxy-phenyl)-phenyl-methyl]piperidine-1-carbonyl]-6-methyl-1-oxo-isoindolin-2-yl]-5-oxo-pentanoate (97.0 mg, 0.140 mmol) in dry acetonitrile (1.3 mL) was added 4-methylbenzenesulfonic acid hydrate (107 mg, 0.561 mmol). The reaction mixture was stirred at 90° C. for 2 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure until the evaporation of most of the acetonitrile. The solution was basified using as saturated aqueous solution of NaHCO₃. The resulting precipitate was filtered, washed with water, and dried under reduced pressure at 50° C. for 72 h to afford (3S)-3-[5-[4-[rel-(S)-(2-chloro-4-fluoro-3-methoxy-phenyl)-phenyl-methyl]piperidine-1-carbonyl]-6-methyl-1-oxo-isoindolin-2-yl]piperidine-2,6-dione as a white powder (33.9 mg, 99% p., 39% y.). m/z [M+H]⁺=618.5, ¹H NMR (500 MHz, DMSO-d₆): δ (ppm) 10.98 (s, 1H), 7.63-7.60 (m, 1H), 7.54-7.42 (m, 2H), 7.37-7.29 (m, 4H), 7.28-7.13 (m, 2H), 5.13-5.07 (m, 1H), 4.53 (br s, 1H), 4.47-4.38 (m, 1H), 4.31 (br d, J=17.5 Hz, 1H), 4.16-4.10 (m, 1H), 3.87-3.76 (m, 3H), 3.30-3.20 (m, 1H), 3.04-2.86 (m, 2H), 2.81-2.70 (m, 1H), 2.63-2.52 (m, 2H), 2.49-2.31 (m, 2H), 2.25 (s, 2H), 2.03-1.96 (m, 1H), 1.68-0.87 (m, 4H), [α]_{D}=−15°·mL·dm⁻¹ g⁻¹[20° C., Na lamp, 589 nm, C=1.2 mg/mL, EtOH].

### Example 114

### (3S)-3-[5-[4-[[3-(2-aminoethoxy)-2-bromo-phenyl]-phenyl-methyl]piperidine-1-carbonyl]-6-methyl-1-oxo-isoindolin-2-yl]piperidine-2,6-dione hydrochloride

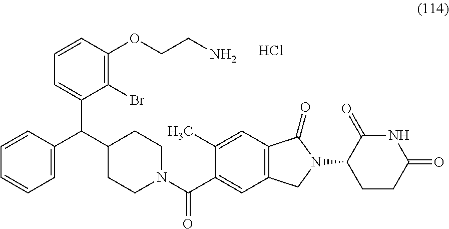 [SMILES: Cc1cc2c(cc1C(=O)N1CCC(C(c3ccccc3)c3cccc(OCCN)c3Br)CC1)CN([C@H]1CCC(=O)NC1=O)C2=O ; Cl]

### Intermediate 114A: 2-bromo-3-tetrahydropyran-2-yloxy-benzaldehyde

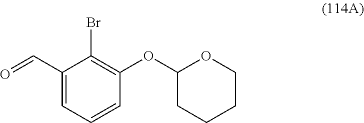 [SMILES: O=Cc1cccc(OC2CCCCO2)c1Br]

To a stirred solution of 2-bromo-3-hydroxybenzaldehyde (3.00 g, 14.9 mmol) in DCM (15 mL) at ambient temperature was added 4-methylbenzenesulfonic acid pyridine (383 mg, 1.49 mmol) and 3,4-dihydro-2H-pyran (2.0 mL, 22.4 mmol). The reaction mixture was stirred at ambient temperature overnight. The reaction mixture was poured in a saturated aqueous solution of NaHCO₃. The mixture was extracted with EtOAc (×2). The combined organic layers were washed with water and brine, dried over Na₂SO₄, filtered, and concentrated under reduced pressure. The crude was purified by flash chromatography on silica gel using 100% toluene. The desired fractions were combined, concentrated, and dried under reduced pressure to afford 2-bromo-3-tetrahydropyran-2-yloxy-benzaldehyde as a white waxy solid (3.16 g, 94% p., 70% y.). m/z [M+H]⁺=no ionization, 1H NMR (400 MHz, DMSO-d₆): δ (ppm) 10.29 (s, 1H), 7.56-7.43 (m, 3H), 5.75 (t, J=3.0 Hz, 1H), 3.71 (ddd, J=11.2, 9.9, 3.6 Hz, 1H), 3.57 (dtd, J=11.2, 4.0, 1.3 Hz, 1H), 1.99 (dtt, J=12.8, 10.2, 5.0 Hz, 1H), 1.86 (ddd, J=10.1, 4.2, 3.2 Hz, 2H), 1.73-1.53 (m, 3H).

### Intermediate 114B: benzyl 4-(p-tolylsulfonylhydrazono) piperidine-1-carboxylate

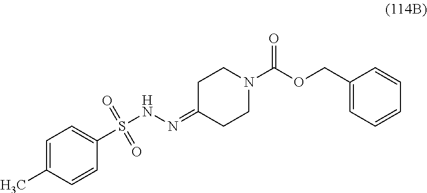 [SMILES: Cc1ccc(S(=O)(=O)NN=C2CCN(C(=O)OCc3ccccc3)CC2)cc1]

To a round-bottom flask, was added 4-methylbenzenesulfonohydrazide (2.32 g, 12.5 mmol) to a stirred solution of benzyl 4-oxopiperidine-1-carboxylate (2.6 mL, 12.5 mmol) in MeOH (25 mL). The reaction mixture was stirred at ambient temperature overnight. The reaction mixture was concentrated and dried under reduced pressure to afford benzyl 4-(p-tolylsulfonylhydrazono) piperidine-1-carboxylate as a white solid (4.29 g, 93% p., 80% y.). The crude was directly used in the next step without further purification. m/z [M+H]⁺=402.4, ¹H NMR (400 MHz, DMSO-d₆): δ (ppm) 10.27 (s, 1H), 7.75-7.68 (m, 2H), 7.42-7.27 (m, 7H), 5.08 (s, 2H), 3.53-3.39 (m, 4H), 2.39 (d, J=8.2 Hz, 5H), 2.25 (dd, J=7.1, 5.1 Hz, 2H).

### Intermediate 114C: benzyl 4-(2-bromo-3-tetrahydropyran-2-yloxy-benzoyl) piperidine-1-carboxylate

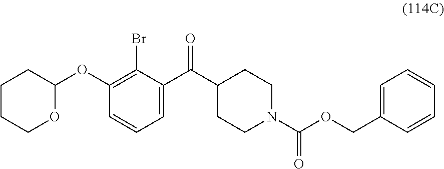 [SMILES: O=C(c1cccc(OC2CCCCO2)c1Br)C1CCN(C(=O)OCc2ccccc2)CC1]

In a sealed vial were combined benzyl 4-(p-tolylsulfonylhydrazono) piperidine-1-carboxylate (4.29 g, 10.7 mmol), 2-bromo-3-tetrahydropyran-2-yloxy-benzaldehyde (3.16 g, 11.1 mmol) and Cs₂CO₃ (7.03 g, 21.4 mmol) in 1,4-dioxane (43 mL). The reaction mixture was flushed by cycles of argon and reduced pressure (3×) and stirred at 110° C. for 4 h. The reaction mixture was acidified with a saturated aqueous solution of NH₄Cl and EtOAc was added. The layers were separated and the aqueous layer was extracted with EtOAc. The combined organic layers were washed with brine, dried over Na₂SO₄, filtered, and concentrated under reduced pressure. The crude was purified by flash chromatography on silica gel using a gradient of EtOAc in toluene from 0% to 10%. The desired fractions were combined, concentrated, and dried under reduced pressure to afford benzyl 4-(2-bromo-3-tetrahydropyran-2-yloxy-benzoyl) piperidine-1-carboxylate as a yellow oil (4.98 g, 81% p., 75% y.). m/z [M+H]⁺=502.1, ¹H NMR (400 MHz, DMSO-d₆): δ (ppm) 7.44-7.26 (m, 7H), 7.05 (dd, J=7.5, 1.4 Hz, 1H), 5.69 (t, J=3.0 Hz, 1H), 5.07 (s, 2H), 4.07-3.94 (m, 2H), 3.76-3.66 (m, 1H), 3.61-3.52 (m, 1H), 3.22 (tt, J=11.2, 3.6 Hz, 1H), 2.91 (s, 2H), 2.00-1.88 (m, 1H), 1.88-1.78 (m, 4H), 1.61 (dtd, J=25.3, 8.6, 4.4 Hz, 3H), 1.44 (qd, J=12.0, 4.2 Hz, 2H).

### Intermediate 114D: benzyl 4-[(2-bromo-3-tetrahydropyran-2-yloxy-phenyl)-hydroxy-phenyl-methyl]piperidine-1-carboxylate

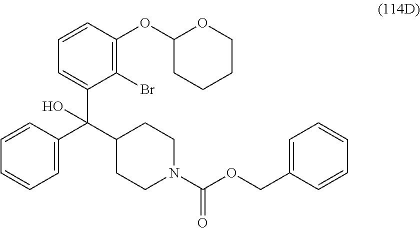 [SMILES: O=C(OCc1ccccc1)N1CCC(C(O)(c2ccccc2)c2cccc(OC3CCCCO3)c2Br)CC1]

In a three-neck round-bottom flask was solubilized benzyl 4-(2-bromo-3-tetrahydropyran-2-yloxy-benzoyl) piperidine-1-carboxylate (4.98 g, 9.91 mmol) in 2-MeTHF (50 mL). The reaction mixture was heated up to 75° C. and was then treated with a 1 M solution of bromo (phenyl) magnesium in THF (50 mL, 49.6 mmol). Directly after the end of the addition, the reaction mixture was cooled to ambient temperature. The reaction was quenched with the addition of water. The mixture was extracted with EtOAc (2×). The combined organic layers were washed with water and brine, dried over Na₂SO₄, filtered, and concentrated under reduced pressure. The crude was purified by flash chromatography on silica gel using a gradient of EtOAc in n-heptane from 0% to 100%. The desired fractions were combined, concentrated, and dried under reduced pressure to afford benzyl 4-[(2-bromo-3-tetrahydropyran-2-yloxy-phenyl)-hydroxy-phenyl-methyl]piperidine-1-carboxylate as a yellow oil (5.90 g, 35% p., 36% y.). m/z [M+H]⁺=580.0.

### Intermediate 114E: benzyl 4-[(2-bromo-3-hydroxy-phenyl)-phenyl-methyl]piperidine-1-carboxylate

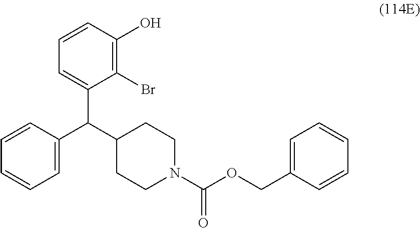 [SMILES: O=C(OCc1ccccc1)N1CCC(C(c2ccccc2)c2cccc(O)c2Br)CC1]

In a round-bottom flask, triethylsilane (19 mL, 0.114 mol) was solubilized in DCM (30 mL) at −25° C. (acetone/dry ice) and TFA (71 mL, 0.927 mol) was added, followed by water (1.8 mL). The reaction mixture was kept at −15° C. and a solution of benzyl 4-[(2-bromo-3-tetrahydropyran-2-yloxy-phenyl)-hydroxy-phenyl-methyl]piperidine-1-carboxylate (5.90 g, 3.56 mmol) in DCM (30 mL) was added dropwise. The reaction mixture was allowed to warm up to 0° C. over 1.25 h. The reaction mixture was poured in a saturated aqueous solution of NaHCO₃. The aqueous layers was extracted with EtOAc (2×). The combined organic layers were washed with water and brine, dried over Na₂SO₄, filtered, and concentrated under reduced pressure. The crude was purified by flash chromatography on silica gel using a gradient of EtOAc in n-heptane from 0% to 50%. The desired fractions were combined, concentrated, and dried under reduced pressure to afford benzyl 4-[(2-bromo-3-hydroxy-phenyl)-phenyl-methyl]piperidine-1-carboxylate as a white foam (1.70 g, 3.33 mmol, 94% p., 94% y.). m/z [M−H]=478.2, ¹H NMR (400 MHz, DMSO-d₆): δ (ppm) 10.14 (s, 1H), 7.40-7.24 (m, 9H), 7.19-7.12 (m, 2H), 7.08 (dd, J=7.9, 1.6 Hz, 1H), 6.76 (dd, J=7.8, 1.5 Hz, 1H), 5.05 (s, 2H), 4.17 (d, J=11.2 Hz, 1H), 3.95 (d, J=13.1 Hz, 2H), 2.72 (d, J=40.8 Hz, 2H), 2.41 (t, J=11.2 Hz, 1H), 1.43 (dd, J=37.2, 13.1 Hz, 2H), 1.10-0.93 (m, 2H).

### Intermediate 114F: 2-bromo-3-[phenyl(4-piperidyl)methyl]phenol hydroiodide

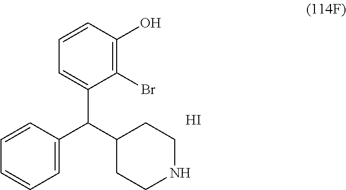

To a stirred solution of benzyl 4-[(2-bromo-3-hydroxy-phenyl)-phenyl-methyl]piperidine-1-carboxylate (500 mg, 0.978 mmol) in dry acetonitrile (9.8 mL) at ambient temperature under nitrogen was added iodo (trimethyl) silane (418 μL, 2.94 mmol) dropwise. The reaction mixture was stirred at ambient temperature for 1 h. MeOH (475 μL, 11.7 mmol) was added dropwise. The mixture was stirred at ambient temperature for 30 min and was concentrated under reduced pressure. Et₂O was added and the resulting mixture was stirred at ambient temperature for 2 h. The solid was filtered, washed with Et₂O, and dried under reduced pressure to afford 2-bromo-3-[phenyl(4-piperidyl)methyl]phenol hydroiodide as a beige powder (468 mg, 89% p., 90% y.). m/z [M+H]⁺=346.2, ¹H NMR (400 MHz, DMSO-d₆): δ (ppm) 10.19 (s, 1H), 8.40 (s, 1H), 8.08 (s, 1H), 7.42-7.35 (m, 2H), 7.30 (t, J=7.6 Hz, 2H), 7.23-7.13 (m, 2H), 7.04 (dd, J=7.9, 1.5 Hz, 1H), 6.78 (dd, J=8.0, 1.5 Hz, 1H), 4.18 (d, J=11.2 Hz, 1H), 3.23 (d, J=12.8 Hz, 2H), 2.82 (t, J=12.7 Hz, 2H), 2.54 (d, J=11.5 Hz, 1H), 1.67-1.41 (m, 2H), 1.38-1.14 (m, 2H).

### Intermediate 114G: tert-butyl(4S)-5-amino-4-[5-[4-[(2-bromo-3-hydroxy-phenyl)-phenyl-methyl]piperidine-1-carbonyl]-6-methyl-1-oxo-isoindolin-2-yl]-5-oxo-pentanoate

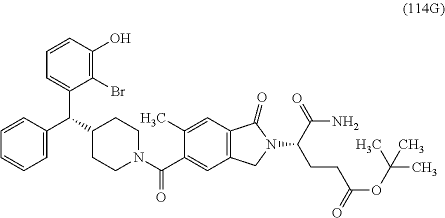 [SMILES: Cc1cc2c(cc1C(=O)N1CCC([C@H](c3ccccc3)c3cccc(O)c3Br)CC1)CN([C@@H](CCC(=O)OC(C)(C)C)C(N)=O)C2=O]

To a solution of 2-bromo-3-[phenyl(4-piperidyl)methyl]phenol hydroiodide (466 mg, 0.875 mmol) in DMF (5.1 mL), were added 2-[(1S)-4-tert-butoxy-1-carbamoyl-4-oxo-butyl]-6-methyl-1-oxo-isoindoline-5-carboxylic acid (362 mg, 0.962 mmol), DIPEA (764 μL, 4.37 mmol) and HATU (514 mg, 1.31 mmol). The mixture was stirred at ambient temperature for 30 min. The reaction was quenched with the addition of a saturated aqueous solution of NH₄Cl. EtOAc was added. The layers were separated and the aqueous layer was extracted with EtOAc (2×). The combined organic layers were washed with brine, dried over Na₂SO₄, filtered, and concentrated under reduced pressure. The crude was purified by flash chromatography on silica gel using a gradient of EtOAc/EtOH (3/1) in cyclohexane from 5% to 100%. The desired fractions were combined and concentrated under reduced pressure to afford tert-butyl(4S)-5-amino-4-[5-[4-[(2-bromo-3-hydroxy-phenyl)-phenyl-methyl]piperidine-1-carbonyl]-6-methyl-1-oxo-isoindolin-2-yl]-5-oxo-pentanoate as a white foam (366 mg, 95% p., 56% y.). m/z [M+H]⁺=704.6, ¹H NMR (400 MHz, DMSO-d₆): δ (ppm) 10.13 (s, 1H), 7.67-6.93 (m, 10H), 6.83-6.67 (m, 1H), 4.77-4.67 (m, 1H), 4.64-4.34 (m, 3H), 4.26-4.14 (m, 1H), 3.24 (d, J=16.7 Hz, 1H), 2.97 (d, J=11.8 Hz, 1H), 2.73 (d, J=0.6 Hz, 1H), 2.51 (s, 1H), 2.23 (t, J=37.2 Hz, 6H), 1.99 (s, 1H), 1.68-0.88 (m, 14H).

### Intermediate 114H: tert-butyl(4S)-5-amino-4-[5-[4-[2-bromo-3-[2-(tert-butoxycarbonylamino) ethoxy]phenyl]-phenyl-methyl]piperidine-1-carbonyl]-6-methyl-1-oxo-isoindolin-2-yl]-5-oxo-pentanoate

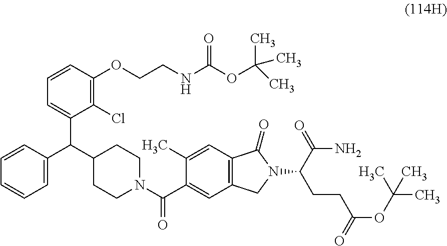 [SMILES: Cc1cc2c(cc1C(=O)N1CCC(C(c3ccccc3)c3cccc(OCCNC(=O)OC(C)(C)C)c3Cl)CC1)CN([C@@H](CCC(=O)OC(C)(C)C)C(N)=O)C2=O]

To a stirred solution of tert-butyl(4S)-5-amino-4-[5-[4-[(2-bromo-3-hydroxy-phenyl)-phenyl-methyl]piperidine-1-carbonyl]-6-methyl-1-oxo-isoindolin-2-yl]-5-oxo-pentanoate (366 mg, 0.493 mmol) in dry THF (3.3 mL) were added successively triphenylphosphane (388 mg, 1.48 mmol), tert-butyl(2-hydroxyethyl) carbamate (229 μL, 1.48 mmol), and then DIAD (291 μL, 1.48 mmol). The reaction mixture was stirred at ambient temperature for 1.5 h. The reaction mixture was concentrated under reduced pressure. The crude was purified by flash chromatography on silica gel using a gradient of EtOAc/EtOH (3/1) (0.5% conc. NH₄OH) in n-heptane from 5% to 100% to afford tert-butyl(4S)-5-amino-4-[5-[4-[[2-bromo-3-[2-(tert-butoxycarbonylamino) ethoxy]phenyl]-phenyl-methyl]piperidine-1-carbonyl]-6-methyl-1-oxo-isoindolin-2-yl]-5-oxo-pentanoate as a white solid (374 mg, 88% p., 79% y.). m/z [M+Na]⁺=869.5.

### Example 114

To a stirred solution of tert-butyl(4S)-5-amino-4-[5-[4-[2-bromo-3-[2-(tert-butoxycarbonylamino) ethoxy]phenyl]-phenyl-methyl]piperidine-1-carbonyl]-6-methyl-1-oxo-isoindolin-2-yl]-5-oxo-pentanoate (100 mg, 0.118 mmol) in dry acetonitrile (1.2 mL) was added 4-methylbenzenesulfonic acid hydrate (292 mg, 1.53 mmol). The reaction mixture was stirred at 90° C. for 6 h. The reaction mixture was cooled to ambient temperature. The reaction was quenched with the addition of a saturated aqueous solution of NaHCO₃. The aqueous layer was extracted with EtOAc (3×). The combined organic layers were washed with brine, dried over Na₂SO₄, filtered, and concentrated under reduced pressure. The crude was purified by reverse-phase preparative chromatography using a gradient of acetonitrile in water from 0% to 100% (0.1% AcOH). The desired fractions were concentrated under reduced pressure until acetonitrile evaporated. A saturated aqueous solution of NaHCO₃ was added and the mixture was extracted with EtOAc (3×). The combined organic layers were dried over Na₂SO₄ and concentrated under reduced pressure. The residue was solubilized in Et₂O(2.0 mL) and treated with a 3 M solution of HCl in CPME (118 μL, 0.354 mmol). The mixture was stirred at ambient temperature for 30 min The resulting precipitate was filtered, washed with Et₂O, and dried under reduced pressure overnight to afford (3S)-3-[5-[4-[3-(2-aminoethoxy)-2-bromo-phenyl]-phenyl-methyl]piperidine-1-carbonyl]-6-methyl-1-oxo-isoindolin-2-yl]piperidine-2,6-dione hydrochloride as a white solid (2.0 mg, 97.8% p., 2% y.). m/z [M+H]⁺=673.2, ¹H NMR (600 MHz, DMSO-d₆): δ (ppm) 11.00 (s, 1H), 8.13-7.77 (m, 3H), 7.61 (br d, J=9.4 Hz, 1H), 7.54-7.06 (m, 8H), 7.01-6.88 (m, 1H), 5.19-5.00 (m, 1H), 4.65-4.03 (m, 6H), 3.29-3.12 (m, 3H), 3.07-2.66 (m, 3H), 2.64-2.53 (m, 2H), 2.46-2.15 (m, 4H), 2.05-1.91 (m, 1H), 1.73-0.69 (m, 4H).

### BIOLOGICAL ASSAYS

The pharmacological properties of the compounds of this invention may be confirmed by a number of biological assays. The exemplified biological assays, which follow, have been carried out with compounds of the invention.

TABLE 1
Degradation Activity and Selectivity

| | CDK2 HIBIT | | 2D Proliferation | | pRB HTRF | | ePL GSPT1 | | Selectivity |
|---|---|---|---|---|---|---|---|---|---|
| | Assay | | Assay | | Assay | | Assay | | Ratio* |
| Ex. | EC₅₀ | Ymin | EC₅₀ | Ymin | EC₅₀ | Ymin | EC₅₀ | Ymin | GSPT1 EC₅₀/ |
| No. | (nM) | % | (nM) | % | (nM) | % | (nM) | % | CDK2 EC₅₀ |
| 1 | 0.6 | 12 | 465.5 | 33 | - | - | 14.9 | 47 | 24.8 |
| 2 | 1.1 | 12 | 2813.7 | 33 | - | - | >10000 | 71 | >9091 |
| 3 | 10.6 | 29 | - | - | - | - | >10000 | 74 | >943 |
| 4 | 13.4 | 26 | - | - | - | - | >10000 | 89 | >746 |
| 5 | 0.3 | 14 | 19.2 | 16 | - | - | >10000 | 61 | >33300 |
| 6 | 68.6 | 54 | - | - | - | - | >10000 | 92 | >146 |
| 7 | 26.3 | 30 | - | - | - | - | >10000 | 73 | >380 |
| 8 | 13.4 | 44 | >10000 | 58 | - | - | >10000 | 68 | >746 |
| 9 | 0.3 | 14 | 8.1 | 8 | 9 | 6 | 18.4 | 40 | 61 |
| 10 | 7 | 19 | >10000 | - | >1000 | - | >10000 | 62 | >1429 |
| 11 | 4 | 15 | 620.8 | 36 | 943.7 | 49 | >10000 | 95 | >2500 |
| 12 | 0.2 | 13 | 5.3 | 8 | 6.9 | 10 | >10000 | 83 | >50000 |
| 13 | 0.7 | 14 | 20.6 | 11 | 19.6 | 10 | >10000 | 86 | >14300 |
| 14 | 5 | 16 | 521 | 14 | 274.6 | 26 | >10000 | 85 | >2000 |
| 15 | 2.4 | 15 | 262.1 | 9 | 378.9 | 28 | >10000 | 75 | >4167 |
| 16 | 17 | 24 | >10000 | 64 | >1000 | 59 | >10000 | 88 | >588 |
| 17 | 1.5 | 15 | 5 | 7 | 8.5 | 12 | >10000 | 58 | >6667 |
| 18 | 2.2 | 14 | 100.3 | 12 | 137 | 25 | >10000 | 94 | >4550 |
| 19 | 0.1 | 15 | 0.8 | 10 | 1.5 | 13 | >10000 | 87 | >100000 |
| 20 | 0.5 | 14 | 6 | 10 | 15.1 | 15 | >10000 | 91 | >20000 |
| 21 | 4.5 | 11 | 346.9 | 11 | 247.5 | 25 | >10000 | 86 | >2222 |
| 22 | 4.4 | 14 | - | - | 960.3 | 45 | - | - | - |
| 23 | 30.7 | 30 | 38.3 | 10 | 59.2 | 16 | >10000 | 106 | >326 |
| 24 | 55.1 | 35 | 10030 | 66 | >1000 | | >10000 | 80 | >182 |
| 25 | 4.9 | 19 | 209.7 | 19 | 143.1 | 41 | >10000 | 103 | >2041 |
| 26 | 1.2 | 15 | 20.7 | 11 | 42 | 12 | >10000 | 89 | >8333 |
| 27 | 0.3 | 18 | 1.5 | 9 | 4.8 | 12 | >10000 | 75 | >33333 |
| 28 | 0.3 | 16 | 8.7 | 13 | 13.9 | 16 | >10000 | 69 | >33333 |
| 29 | 1.2 | 18 | 25.4 | 11 | 21.1 | 11 | >10000 | 91 | >8333 |
| 30 | 0.5 | 15 | 7.5 | 12 | 15.5 | 16 | >10000 | 85 | >20000 |
| 31 | 0.3 | 15 | 3 | 12 | 2.7 | 10 | >10000 | 95 | >33333 |
| 32 | 2.9 | 18 | 4032.7 | 45 | >1000 | 55 | >10000 | 96 | >3448 |
| 33 | 58 | 42 | >10000 | 63 | >1000 | | >10000 | 84 | >172 |
| 34 | 1.4 | 13 | 60.2 | 22 | 197.3 | 34 | >10000 | 91 | >7143 |
| 35 | 88 | 47 | >10000 | | >1000 | | >10000 | 84 | >114 |
| 36 | 12.2 | 22 | 128.1 | 12 | 70 | 9 | >10000 | 95 | >820 |
| 37 | 94.2 | 47 | 9721 | 57 | >1000 | 45 | >10000 | 71 | >106 |
| 38 | 65.8 | 41 | 8468.2 | 51 | >1000 | 52 | >10000 | 69 | >152 |
| 39 | 46.2 | 40 | - | - | >1000 | 69 | - | - | - |
| 40 | 4.9 | 18 | 50.5 | 15 | 81.8 | 8 | >10000 | 83 | >2041 |
| 41 | 0.9 | 15 | 10.3 | 18 | 4.8 | 9 | >10000 | 120 | >11111 |
| 42 | 1.4 | 16 | 14.2 | 14 | 12.5 | 4 | >10000 | 72 | >7143 |
| 43 | 19.3 | 27 | 242.9 | 20 | 240.7 | 25 | >10000 | 75 | >518 |
| 44 | 22.4 | 29 | 181.7 | 19 | 296.1 | 23 | >10000 | 85 | >446 |
| 45 | 5.9 | 16 | 19.7 | 10 | 24.3 | 2 | >10000 | 78 | >1695 |
| 46 | 5 | 17 | 25.3 | 16 | 9.7 | 12 | >10000 | 68 | >2000 |
| 47 | 1.1 | 15 | 12 | 12 | 11.5 | 6 | >10000 | 85 | >9091 |
| 48 | 1.5 | 18 | 41.2 | 19 | 22 | 12 | >10000 | 85 | >6667 |
| 49 | 37.6 | 33 | 136.7 | 33 | >1000 | 49 | >10000 | 80 | >266 |
| 50 | 3 | 22 | 7.6 | 15 | 833.4 | 62 | >10000 | 59 | >3333 |
| 51 | 0.2 | 15 | 4 | 11 | 6.8 | 12 | >10000 | 52 | >50000 |
| 52 | 0.5 | 13 | 365.2 | 1 | 33.8 | 44 | >10000 | 52 | >20000 |
| 53 | 9.2 | 18 | 234.2 | 16 | 444.3 | 29 | >10000 | 64 | >1087 |
| 54 | 0.4 | 13 | 14.4 | 43 | 13.4 | 36 | >10000 | 64 | >25000 |
| 55 | 0.5 | 13 | 11.2 | 42 | 17.1 | 37 | >10000 | 66 | >20000 |
| 56 | 28.1 | 29 | 698.5 | 28 | 105 | 17 | >10000 | 67 | >356 |
| 57 | 1.4 | 11 | 54.7 | 11 | 33.2 | 11 | >10000 | 98 | >71423 |
| 58 | 46.8 | 34 | 561.6 | 25 | 398 | 23 | >10000 | 74 | >214 |
| 59 | 0.4 | 16 | 20.7 | 12 | 10.9 | 19 | >10000 | 88 | >25000 |
| 60 | 0.1 | 13 | 1.2 | 16 | 2.1 | 11 | >10000 | 101 | >100000 |
| 61 | 7.7 | 18 | 32 | 9 | 41.7 | 7 | >10000 | 76 | >1299 |
| 62 | 20.3 | 24 | 1563.9 | 11 | 969.7 | 50 | >10000 | 95 | >493 |
| 63 | 2.9 | 16 | 168.9 | 15 | 181.5 | 28 | >10000 | 93 | >3448 |
| 64 | 62.9 | 35 | 1770.1 | 28 | >1000 | 50 | >10000 | 62 | >159 |
| 65 | 0.1 | 15 | 1.7 | 15 | 2.8 | 9 | >10000 | 63 | >100000 |
| 66 | 5.5 | 10 | 40.6 | 15 | 50.2 | 13 | >10000 | 84 | >1818 |
| 67 | 4.2 | 15 | 41.7 | 8 | 37.6 | 8 | >10000 | 96 | >2381 |
| 68 | 33.6 | 29 | 2054.5 | 32 | 877 | 49 | >10000 | 95 | >298 |
| 69 | 1.2 | 15 | 95.1 | 15 | 164.4 | 30 | >10000 | 94 | >8333 |
| 70 | 1.1 | 16 | 66.5 | 29 | 19.4 | 43 | >10000 | 90 | >9091 |
| 71 | 3.3 | 16 | 29.8 | 5 | 44.1 | 11 | >10000 | 98 | >3030 |
| 72 | 0.6 | 15 | 36.4 | 24 | 34.1 | 35 | >10000 | 70 | >16667 |
| 73 | 1.8 | 13 | 27.2 | 13 | 23.5 | 13 | >10000 | 89 | >5556 |
| 74 | 0.2 | 12 | 3.2 | 19 | 3.5 | 16 | >10000 | 92 | >50000 |
| 75 | 0.2 | 12 | 2.8 | 13 | 2.5 | 13 | >10000 | 96 | >50000 |
| 76 | 4.4 | 15 | >10000 | 74 | >1000 | 68 | >10000 | 76 | >2273 |
| 77 | 1.5 | 13 | 93.9 | 18 | 47.2 | 13 | >10000 | 102 | >6667 |
| 78 | 0.8 | 15 | 2510.1 | 0 | >1000 | 62 | >10000 | 70 | >12500 |
| 79 | 6.7 | 14 | 20 | 25 | 12.5 | 17 | >10000 | 81 | >1493 |
| 80 | 10.8 | 17 | 26 | 21 | 30.9 | 5 | >10000 | 109 | >926 |
| 81 | 0.6 | 14 | 18.5 | 30 | 12.9 | 32 | >10000 | 106 | >16667 |
| 82 | 96 | 46 | >10000 | - | >1000 | - | >10000 | 108 | >104 |
| 83 | 0.7 | 11 | 2251.9 | 29 | 666.3 | 47 | >10000 | 97 | >14286 |
| 84 | 59.5 | 42 | >10000 | - | >1000 | - | >10000 | 59 | >168 |
| 85 | 2.3 | 12 | 968 | 21 | 157.9 | 31 | >10000 | 81 | >4348 |
| 86 | 3.6 | 10 | 65.7 | 21 | 103.8 | 13 | >10000 | 79 | >2778 |
| 87 | 0.6 | 16 | 23.1 | 40 | 17.6 | 27 | >10000 | 91 | >16667 |
| 88 | 0.3 | 14 | 7.8 | 17 | 6.7 | 15 | >10000 | 129 | >33333 |
| 89 | 0.2 | 16 | 3.8 | 13 | 4.2 | 10 | >10000 | 112 | >50000 |
| 90 | 0.7 | 19 | - | - | >1000 | - | >10000 | 61 | >14286 |
| 91 | 0.2 | 14 | 2.2 | 12 | 3.7 | 11 | >10000 | 145 | >50000 |
| 92 | 0.2 | 15 | 2.6 | 15 | 3.3 | 8 | >10000 | 82 | >50000 |
| 93 | 2.3 | 17 | 282 | 23 | 109.4 | 23 | >10000 | 56 | >4348 |
| 94 | 0.6 | 14 | 6.8 | 7 | 5.1 | 10 | >10000 | 108 | >16667 |
| 95 | 33.7 | 30 | 30.5 | 17 | 14.1 | 9 | >10000 | 97 | >297 |
| 96 | 4 | 17 | 1285.9 | 11 | 444.9 | 38 | >10000 | 65 | >2500 |
| 97 | 5.8 | 21 | >10000 | - | >1000 | 61 | >10000 | 68 | >1724 |
| 98 | 0.2 | 15 | 5.1 | 11 | 5.5 | 13 | >10000 | 72 | >50000 |
| 99 | 10 | 21 | 79.5 | 19 | 23.4 | 8 | >10000 | 88 | >1000 |
| 100 | 0.1 | 13 | 2.7 | 9 | 3.1 | 13 | >10000 | 73 | >100000 |
| 101 | 0.2 | 15 | 2.7 | 10 | 3.5 | 11 | >10000 | 82 | >50000 |
| 102 | 0.2 | 16 | 14 | 20 | 3.4 | 29 | >10000 | 64 | >50000 |
| 103 | 0.7 | 15 | 15.6 | 16 | 11.7 | 10 | >10000 | 52 | >14286 |
| 104 | 13.1 | 24 | 4338.3 | 46 | >1000 | 54 | 7699 | 51 | 588 |
| 105 | 1.8 | 14 | 2573.2 | 47 | 342.2 | 36 | >10000 | 99 | >5556 |
| 106 | 2.7 | 18 | >10000 | 69 | >1000 | 58 | >10000 | 63 | >3704 |
| 107 | 41.9 | 36 | >10000 | - | >1000 | - | >10000 | 72 | >239 |
| 108 | 0.9 | 13 | 114.1 | 16 | 56.1 | 25 | >10000 | 71 | >11111 |
| 109 | 50.6 | 38 | >10000 | - | >1000 | - | >10000 | 101 | >198 |
| 110 | 0.2 | 15 | 5 | 9 | 2.7 | 14 | >10000 | 67 | >50000 |
| 111 | 2.5 | 15 | 843.5 | 12 | 490.9 | 37 | >10000 | 49 | >4000 |
| 112 | 47.5 | 39 | 654.1 | 0 | 725.6 | 42 | 7977 | 51 | 168 |
| 113 | 1.6 | 19 | 2678.3 | 20 | >1000 | 59 | >10000 | 68 | >6250 |
| 114 | 5.6 | 15 | 50.8 | 14 | 23.7 | 8 | - | - | - |

*Selectivity Ratio is the ratio of the GSPT1 EC₅₀ value divided by the
CDK2 HiBiT EC₅₀ value;
Ymin % is the percentage of protein remaining.

Table 1 shows the activities of Examples 1 to 114 for the degradation of CDK2 (CDK2 HiBiT Assay), inhibition of proliferation (2D proliferation assay), inhibition of pRB (pRB HTRF Assay), and degradation of GSPT1 (ePL GSPT1 Assay). A smaller EC₅₀ value and smaller Ymin value in the CDK2 assay indicated greater activity for the compound in the CDK2 assay. A larger EC₅₀ value and larger Ymin value in the CDK2 assay indicated less activity for the compound in the CDK2 assay. A smaller EC₅₀ value and smaller Ymin value in the 2D proliferation assay indicated greater activity for the compound in the 2D proliferation assay. A larger EC₅₀ value and larger Ymin value in the 2D proliferation assay indicated less activity for the compound in the 2D proliferation assay. A smaller EC₅₀ value and smaller Ymin value in the pRB assay indicated greater activity for the compound in the pRB assay. A larger EC₅₀ value and larger Ymin value in the pRB assay indicated less activity for the compound in the pRB assay. A smaller EC₅₀ value and smaller Ymin value in the GSPT1 assay indicated greater activity for the compound in the GSPT1 assay. A larger EC₅₀ value and larger Ymin value in the GSPT1 assay indicated less activity for the compound in the GSPT1 assay. A larger value for the ratio of GSPT1 EC₅₀/CDK2 EC₅₀ indicated that a compound had greater selectivity for activity towards CDK2 degradation than activity towards GSPT1 degradation, and thus was more selective over GSPT1 degradation.

In the study reported in Table 1, the exemplified compounds, Examples 1-21, 23-38, and 40-113 showed selectivity of at least 24 times for activity towards degradation of CDK2 over activity towards degradation of GSPT1.

The compounds of the present invention possess activity as degraders of CDK2 and are selective over degradation of GSPT1. Thus, the compounds of the present invention may be used in the treatment of cancer and other proliferative diseases while reducing or minimizing side effects associated with degradation of GSPT1. Thus, the present invention fills the foregoing need by providing compounds that are useful to decrease the level of CDK2 and are selective against degradation of GSPT1.

### CDK2 HiBiT Assay

The HEK293 FT/GSPT1-G575N KI/CRBN OE/CDK2-HiBiT cell line was generated using CRISPR-Cas9 to knock-in the GSPT1-G575N mutation followed by overexpression of CRBN and CDK2-HiBiT via lentiviral transduction and puromycin selection, and genetic edition was further validated using Nano-Glo® HiBiT blotting system. HEK293 CDK2-HiBiT cells were maintained in culture medium (DMEM+10% FBS, Pyruvate Sodium 1×, HEPES, Glutamax, non-essential amino acids, Penicillin/Streptomycin) at 37° C. and 5% CO₂. For the assay, cells were seeded at 5000 cells/well in white 384 viewplates (Revvity #6007480) in 22.5 μL of culture medium without phenol red. Test compounds were spotted at in 384 Deep-well plates (Greiner Bio-one #7F4201) by an acoustic dispenser (10 concentrations, semi-log dilution, in 100% DMSO). Before treatment, compounds were diluted to 10× in culture medium without phenol red, and cells were treated with 2.5 μL of diluted compounds 18-24 h after seeding. After a quick spin, plates were incubated at 37° C. and 5% CO₂ for 4 to 24 hours. Plates and Nano-Glo® HiBiT buffer were equilibrated at room temperature at the end of the treatment. Nano-Glo® HiBiT Lytic reagent was prepared and 25 μL of Nano-Glo® HiBiT mix were added per well according to the manufacturer's instruction (Promega, #N3040). Luminescence was measured on a PHERAstar FSX (BMGLab Tech) plate reader 10 min after addition of the detection reagent. Dose responses were then analyzed using the Genedata Screener® Software. Normalization was performed using the DMSO treated wells as Neutral Control and medium only (without cells) wells as Scale Reference. For curve fitting, the smart fit condensing method with a 4-parameters Hill model was used.

### pRB HTRF Assay

HCC1569 cells were amplified in culture medium (RPMI1640 ATCC modified, 10% SVF, Penicillin/Streptomycin) at 37° C. and 5% CO₂. For the pRB inhibition assay, cells were seeded at 5000 cells/well in 384 Well CulturPlate (Revvity #6007688) in 22.5 μL of culture medium. Test compounds were spotted in 384 Deep-well plates (Greiner Bio-one #7F4201) by an acoustic dispenser in 100% DMSO. Compounds were diluted to 10× culture medium and 2.5 μL of diluted compounds were added on cells 18 to 24 hours after seeding. After a quick spin, plates were incubated at 37° C. and 5% CO₂. HTRF Human and Mouse Phospho-Rb (Ser807/811) Detection Kit (Revvity, 64RBS807PEH) was used to detect pRB levels 48 hr after treatment. Briefly, a Lysis/antibody mix was prepared with 12 μL water, 4 μL lysis buffer, 4 μL antibody mix and 0.2 μL de protease inhibitor per well. Medium was removed from culture plates by flicking and 20 μL of Lysis/Antibody mix was added to each well. After a quick spin, the plates were placed 5 min on a shaker and incubated for 4 hr at room temperature in the dark. FRET signals (620 nm and 665 nm) were detected on a PHERASTAR FSX plate reader (BMG LabTech) with TRF module. Dose responses were then analyzed using the Genedata Screener® Software. Normalization was performed using the DMSO treated wells as Neutral Control and medium only (without cells) as Scale Reference. For curve fitting, the smart fit condensing method with a 4-parameters Hill model was used.

### 2D Proliferation Assay

HCC1569 cells were amplified in culture medium (RPMI1640 ATCC modified, 10% SVF, Penicillin/Streptomycin) at 37° C. and 5% CO₂. For the proliferation assay, cells were seeded at 600 cells/well in 384 Well Black ViewPlate (Revvity #6007460) in 72 μL of culture medium. Test compounds were spotted in 384 Deep-well plates (Greiner Bio-one #7F4201) by an acoustic dispenser (10 concentrations, semi-log dilution, in 100% DMSO). Compounds were diluted to 10× culture medium and 8 μL of diluted compounds were added on cells 18 to 24 hours after seeding. After a quick spin plates were covered with a MicroClime® (Labcyte #LLS0310) lid to avoid evaporation during incubation. Seven days after seeding, cells were fixed in 4% PFA, washed with PBS, and nuclei were stained with Hoechst. Images were taken with Operetta® High Content Imaging System (Perkin Elmer) at non confocal 10× magnification, 4 fields per well. Analysis of images for nuclei counting was performed with Columbus™ Image Data Storage and Analysis System. Dose responses were then analyzed using the Genedata Screener® Software. Normalization was performed using the DMSO treated wells as Neutral Control and cells treated with PF-07104091 at 10 μM as Scale Reference. For curve fitting, the smart fit condensing method with a 4-parameters Hill model was used.

### ePL GSPT1 Assay

The DF15 cells GSPT1-ePL over expressing cell line was generated using the lentiviral transduction and blasticidin selection. GSPT1-ePL/DF15 cells were maintained in culture medium (RPMI 1640, 10% FBS, Sodium-pyruvate 1×, Pen/Strep 1×, Blasticidin 4 μg/ml) at 37° C. and 5% CO₂. Next, 25 nL of test compounds were spotted at in 384 well plates (Greiner #781073) by Labcyte Echo acoustic dispenser (10 or 8 concentrations, semi log dilution, in 100% DMSO). For the assay, 25 L of cells in assay medium (RPMI1640, 10% FBS) were seeded at 3000 cells/well directly on compounds plates. After a quick spin plates were incubated at 37° C. and 5% CO₂ for 20 hours. Plates and ePL detection reagents (InCELL Hunter ePL #96-0079) were equilibrated 30 min at 20° C. at the end of the treatment. ePL reagents were added to the well and plates were incubated 1 h at 20° C. in the dark. ePL signal was measured on a TECAN Spark plate reader. Dose responses were then analyzed using the Genedata Screener® Software. Normalization was performed using the DMSO treated wells as Neutral Control and Ataluren (PTC-124) as scale reference. For curve fitting, the smart fit condensing method with a 4-parameters Hill model was used.

What is claimed is:

1. A compound of Formula (I): 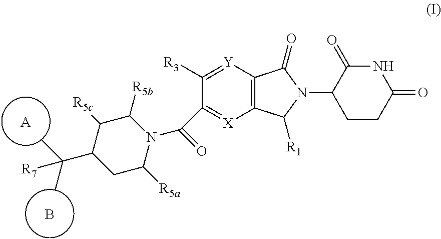
or stereoisomers, tautomers, or salts thereof, wherein:
(i) X is CR₂ and Y is CR₄;
(ii) X is CR₂ and Y is N;
(iii) X is N and Y is N;
R₁ is hydrogen or —CH₃;
R₂ is hydrogen or —CH₃;
R₃ is hydrogen, F, —CH₃, —CHF₂, or —CH₂N(CH₃)₂;
R₄ is hydrogen;
wherein:
(i) R₅ₐ, R_{5b}, and R_{5c} are each hydrogen;
(ii) R₅ₐ and R_{5b} are joined together to form-CH₂CH₂-or —CH₂OCH₂—, and R_{5c} is hydrogen; or
(iii) R₅ₐ and R_{5c} are joined together to form-CH₂—, and R_{5b} is hydrogen;
Ring A is: 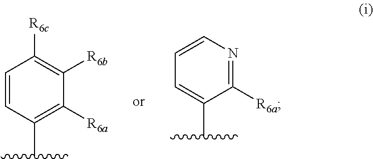
or
(ii) indazolyl, indolinyl, dihydrobenzofuranyl, dihydrobenzoxazinyl, tetrahydroquinolinyl, tetrahydrobenzazepinyl, or tetrahydrobenzooxazepinyl;
Ring B is phenyl, isothiazolyl, 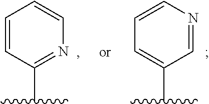 [SMILES: *c1ccccn1 ; *c1cccnc1]
R₆ₐ is hydrogen, Cl, or Br;
R_{6b} is hydrogen, —OH, —OCH₃, —OCD₃, —OCH₂CH₃, —OCH(CH₃)₂, —OCH₂CH₂OH, —OCH₂CH₂OCH₃, —OCF₃, —OCH₂CH₂F, —OCH₂CH₂NH₂, —OCH₂CH₂NH(CH₃), —OCH₂CH₂N(CH₃)₂, —OCH₂CH₂CH₂NH₂, —OCH₂CH₂CH₂N(CH₃)₂, —OCH₂CHFCH₂NH₂, —OCH₂CF₂CH₂NH₂, —OCH(CH₃) CH₂NH₂, —OCH₂CH(CH(CH₃)₂) NH₂, —OCH₂CH(CH₃) NH₂, —OCH₂CH(CH₂CH₃) NH₂, —OCH₂CH(CHF₂) NH₂, —OCH₂CH(CF₃) NH₂, —OCH₂CH(CH₂CHF₂) NH₂, —OCH₂CH₂NHC(O)CH₃, —B(OH)₂, —CH₂CH₂N(CH₃)₂, —NH(CH₃), —N(CH₃)₂, —NH(CH₂CH₂OH), —NH(CH₂CH₂NH₂), —NH (aminocyclobutyl), —NHC(O)CH₃, —NHC(O)CH₂OH, —O(cyclopropyl), —O(cyclobutyl), —O(aminocyclopentyl), —O(methylazetidinyl), —O(methyl pyrrolidinyl), —O(fluoroethyl pyrrolidinyl), —O(trifluoroethyl pyrrolidinyl), —O(methyl azaspiro[3.3]heptanyl), —OCH₂(aminocyclopropyl), —OCH₂(imidazolyl), —OCH₂(methyl imidazolyl), —OCH₂(isoxazolyl), —OCH₂(oxazolyl), —OCH₂(methyl pyrrolidinyl), —OCH₂CH₂(pyrrolidinyl), morpholinyl, oxazolyl, aminopyrrolidinyl, hydroxypyrrolidinyl, or dihydroimidazo[1,5-a]pyrazinyl;
R_{6c} is hydrogen or F; and
R₇ is hydrogen or deuterium.

2. The compound according to claim 1 or stereoisomers, tautomers, or salts thereof, having the structure:

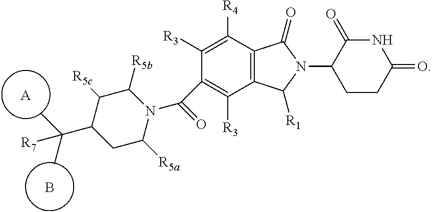

3. The compound according to claim 1 or stereoisomers, tautomers, or salts thereof, having the structure:

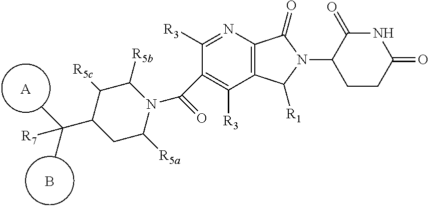

4. The compound according to claim 1 or stereoisomers, tautomers, or salts thereof, having the structure:

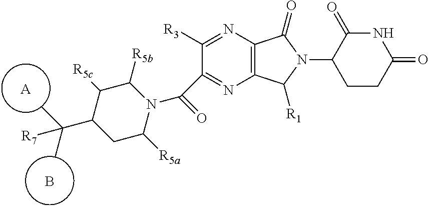

5. The compound according to claim 1 or stereoisomers, tautomers, or salts thereof, wherein R₃ is-CH₃.

6. The compound according to claim 1 or stereoisomers, tautomers, or salts thereof, wherein Ring A is

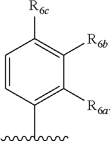

7. The compound according to claim 1 or stereoisomers, tautomers, or salts thereof, wherein Ring B is phenyl.

8. The compound according to claim 1 or stereoisomers, tautomers, or salts thereof, wherein R^{5a}, R_{5b}, and R_{5c} are each hydrogen.

9. The compound according to claim 1 or stereoisomers, tautomers, or salts thereof, wherein:
Ring A is 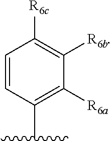
Ring B is phenyl;
R₃ is-CH₃;
R₅ₐ, R_{5b}, and R_{5c} are each hydrogen; and
R₆ₐ is Cl.

10. The compound according to claim 1 or stereoisomers, tautomers, or salts thereof, wherein said compound is:
(3S)-3-[5-(4-benzhydrylpiperidine-1-carbonyl)-1-oxo-isoindolin-2-yl]piperidine-2,6-dione (1);
(3S)-3-[5-(4-benzhydrylpiperidine-1-carbonyl)-6-methyl-1-oxo-isoindolin-2-yl]piperidine-2,6-dione (2);
(3S)-3-[5-[(1R,5S)-3-benzhydryl-8-azabicyclo[3.2.1]octane-8-carbonyl]-1-oxo-isoindolin-2-yl]piperidine-2,6-dione (3);
(3S)-3-[5-(7-benzhydryl-3-oxa-9-azabicyclo[3.3.1]nonane-9-carbonyl)-1-oxo-isoindolin-2-yl]piperidine-2,6-dione (4);
(3S)-3-[5-[4-[(2-chlorophenyl)-phenyl-methyl]piperidine-1-carbonyl]-6-methyl-1-oxo-isoindolin-2-yl]piperidine-2,6-dione (5);
(3S)-3-[5-[(1R,4R)-5-benzhydryl-2-azabicyclo[2.2.1]heptane-2-carbonyl]-1-oxo-isoindolin-2-yl]piperidine-2,6-dione (6);
(3S)-3-[5-[(1R,5S)-3-benzhydryl-8-azabicyclo[3.2.1]octane-8-carbonyl]-6-fluoro-1-oxo-isoindolin-2-yl]piperidine-2,6-dione (7);
(3S)-3-[5-[(1R,5S)-3-benzhydryl-8-azabicyclo[3.2.1]octane-8-carbonyl]-4-methyl-1-oxo-isoindolin-2-yl]piperidine-2,6-dione (8);
(3S)-3-[5-[4-[(R)-(2-chlorophenyl)-phenyl-methyl]piperidine-1-carbonyl]-1-oxo-isoindolin-2-yl]piperidine-2,6-dione (9);
(3S)-3-[5-(4-benzhydrylpiperidine-1-carbonyl)-6-(difluoromethyl)-1-oxo-isoindolin-2-yl]piperidine-2,6-dione (10);
(3S)-3-[3-[4-[(2-chlorophenyl)-phenyl-methyl]piperidine-1-carbonyl]-7-oxo-5H-pyrrolo[3,4-b]pyridin-6-yl]piperidine-2,6-dione (11);
(3S)-3-[5-[4-[(R)-(2-chlorophenyl)-phenyl-methyl]piperidine-1-carbonyl]-6-methyl-1-oxo-isoindolin-2-yl]piperidine-2,6-dione (12);
(3S)-3-[3-[4-[(R)-(2-chloro-3-methoxy-phenyl)-phenyl-methyl]piperidine-1-carbonyl]-7-oxo-5H-pyrrolo[3,4-b]pyridin-6-yl]piperidine-2,6-dione (13);
(3S)-3-[3-[4-[(S)-(2-chloro-3-methoxy-phenyl)-phenyl-methyl]piperidine-1-carbonyl]-7-oxo-5H-pyrrolo[3,4-b]pyridin-6-yl]piperidine-2,6-dione (14);
(3S)-3-[3-[4-[(R)-(2-chlorophenyl)-phenyl-methyl]piperidine-1-carbonyl]-7-oxo-5H-pyrrolo[3,4-b]pyridin-6-yl]piperidine-2,6-dione (15);
(3S)-3-[3-[4-[(S)-(2-chlorophenyl)-phenyl-methyl]piperidine-1-carbonyl]-7-oxo-5H-pyrrolo[3,4-b]pyridin-6-yl]piperidine-2,6-dione (16);
(3R)-3-[5-[4-[(R)-(2-chlorophenyl)-phenyl-methyl]piperidine-1-carbonyl]-6-methyl-1-oxo-isoindolin-2-yl]piperidine-2,6-dione (17);
(3S)-3-[3-[4-[(2-chlorophenyl)-phenyl-methyl]piperidine-1-carbonyl]-2-methyl-7-oxo-5H-pyrrolo[3,4-b]pyridin-6-yl]piperidine-2,6-dione (18);
(3S)-3-[5-[4-[(R)-(2-chloro-3-methoxy-phenyl)-phenyl-methyl]piperidine-1-carbonyl]-6-methyl-1-oxo-isoindolin-2-yl]piperidine-2,6-dione (19);
(3S)-3-[5-[4-[(S)-(2-chloro-3-methoxy-phenyl)-phenyl-methyl]piperidine-1-carbonyl]-6-methyl-1-oxo-isoindolin-2-yl]piperidine-2,6-dione (20);
(3S)-3-[3-[4-[(2-chlorophenyl)-phenyl-methyl]piperidine-1-carbonyl]-2-methyl-7-oxo-5H-pyrrolo[3,4-b]pyrazin-6-yl]piperidine-2,6-dione (21);
(3S)-3-[5-[4-[(2-chlorophenyl)-phenyl-methyl]piperidine-1-carbonyl]-6-[(dimethylamino)methyl]-1-oxo-isoindolin-2-yl]piperidine-2,6-dione (22);
[2-chloro-3-[[1-[2-[(3S)-2,6-dioxo-3-piperidyl]-6-methyl-1-oxo-isoindoline-5-carbonyl]-4-piperidyl]-phenyl-methyl]phenyl]boronic acid (23);
(3S)-3-[5-[4-[(S)-[2-chloro-3-[2-(dimethylamino) ethoxy]phenyl]-phenyl-methyl]piperidine-1-carbonyl]-6-methyl-1-oxo-isoindolin-2-yl]piperidine-2,6-dione hydrochloride (24);
(3S)-3-[5-[4-[(S)-[2-chloro-3-(2-hydroxyethoxy)phenyl]-phenyl-methyl]piperidine-1-carbonyl]-6-methyl-1-oxo-isoindolin-2-yl]piperidine-2,6-dione (25);
(3S)-3-[5-[4-[(R)-[2-chloro-3-[2-(dimethylamino) ethoxy]phenyl]-phenyl-methyl]piperidine-1-carbonyl]-6-methyl-1-oxo-isoindolin-2-yl]piperidine-2,6-dione hydrochloride (26);
(3S)-3-[5-[4-[[2-chloro-3-(methylamino)phenyl]-phenyl-methyl]piperidine-1-carbonyl]-6-methyl-1-oxo-isoindolin-2-yl]piperidine-2,6-dione (27);
(3S)-3-[5-[4-[[2-chloro-3-(dimethylamino)phenyl]-phenyl-methyl]piperidine-1-carbonyl]-6-methyl-1-oxo-isoindolin-2-yl]piperidine-2,6-dione (28);
(3S)-3-[5-[4-[(R)-(2-chloro-3-hydroxy-phenyl)-phenyl-methyl]piperidine-1-carbonyl]-6-methyl-1-oxo-isoindolin-2-yl]piperidine-2,6-dione (29);
(3S)-3-[5-[4-[(2-chloro-3-oxazol-2-yl-phenyl)-phenyl-methyl]piperidine-1-carbonyl]-6-methyl-1-oxo-isoindolin-2-yl]piperidine-2,6-dione (30);
(3S)-3-[5-[4-[(R)-[2-chloro-3-(2-hydroxyethoxy)phenyl]-phenyl-methyl]piperidine-1-carbonyl]-6-methyl-1-oxo-isoindolin-2-yl]piperidine-2,6-dione (31);
(3S)-3-[5-[4-[(2-chlorophenyl)-isothiazol-5-yl-methyl]piperidine-1-carbonyl]-6-methyl-1-oxo-isoindolin-2-yl]piperidine-2,6-dione (32);
(3S)-3-[5-[4-[(S)-[2-chloro-3-[(3R)-1-methylpyrrolidin-3-yl]oxy-phenyl]-phenyl-methyl]piperidine-1-carbonyl]-6-methyl-1-oxo-isoindolin-2-yl]piperidine-2,6-dione hydrochloride (33);
(3S)-3-[5-[4-[(R)-[2-chloro-3-[(3S)-1-methylpyrrolidin-3-yl]oxy-phenyl]-phenyl-methyl]piperidine-1-carbonyl]-6-methyl-1-oxo-isoindolin-2-yl]piperidine-2,6-dione hydrochloride (34);
(3S)-3-[5-[4-[(S)-[2-chloro-3-[(3S)-1-methylpyrrolidin-3-yl]oxy-phenyl]-phenyl-methyl]piperidine-1-carbonyl]-6-methyl-1-oxo-isoindolin-2-yl]piperidine-2,6-dione hydrochloride (35);
N-[2-chloro-3-[[1-[2-[(3S)-2,6-dioxo-3-piperidyl]-6-methyl-1-oxo-isoindoline-5-carbonyl]-4-piperidyl]-phenyl-methyl]phenyl]acetamide (36);
(3S)-3-[5-[4-[(S)-[2-chloro-3-[[(3R)-1-methylpyrrolidin-3-yl]methoxy]phenyl]-phenyl-methyl]piperidine-1-carbonyl]-6-methyl-1-oxo-isoindolin-2-yl]piperidine-2,6-dione hydrochloride (37);
(3S)-3-[5-[4-[(S)-[2-chloro-3-[3-(dimethylamino) propoxy]phenyl]-phenyl-methyl]piperidine-1-carbonyl]-6-methyl-1-oxo-isoindolin-2-yl]piperidine-2,6-dione hydrochloride (38);
(3S)-3-[5-[4-[[2-chloro-3-[2-(dimethylamino)ethyl]phenyl]-phenyl-methyl]piperidine-1-carbonyl]-6-methyl-1-oxo-isoindolin-2-yl]piperidine-2,6-dione hydrochloride (39);
(3S)-3-[5-[4-[(S)-[2-chloro-3-[(3-methylimidazol-4-yl)methoxy]phenyl]-phenyl-methyl]piperidine-1-carbonyl]-6-methyl-1-oxo-isoindolin-2-yl]piperidine-2,6-dione hydrochloride (40);
(3S)-3-[5-[4-[(R)-[2-chloro-3-[(3-methylimidazol-4-yl)methoxy]phenyl]-phenyl-methyl]piperidine-1-carbonyl]-6-methyl-1-oxo-isoindolin-2-yl]piperidine-2,6-dione (41);
(3S)-3-[5-[4-[(R)-[2-chloro-3-(1-methylazetidin-3-yl)oxy-phenyl]-phenyl-methyl]piperidine-1-carbonyl]-6-methyl-1-oxo-isoindolin-2-yl]piperidine-2,6-dione (42);
(3S)-3-[5-[4-[(S)-[2-chloro-3-[(1-methylimidazol-2-yl)methoxy]phenyl]-phenyl-methyl]piperidine-1-carbonyl]-6-methyl-1-oxo-isoindolin-2-yl]piperidine-2,6-dione hydrochloride (43);
3S)-3-[5-[4-[(S)-[2-chloro-3-[(1-methylimidazol-4-yl)methoxy]phenyl]-phenyl-methyl]piperidine-1-carbonyl]-6-methyl-1-oxo-isoindolin-2-yl]piperidine-2,6-dione hydrochloride (44);
(3S)-3-[5-[4-[(R)-[2-chloro-3-[2-(methylamino) ethoxy]phenyl]-phenyl-methyl]piperidine-1-carbonyl]-6-methyl-1-oxo-isoindolin-2-yl]piperidine-2,6-dione hydrochloride (45);
(3S)-3-[5-[4-[(R)-[3-(2-aminoethoxy)-2-chloro-phenyl]-phenyl-methyl]piperidine-1-carbonyl]-6-methyl-1-oxo-isoindolin-2-yl]piperidine-2,6-dione hydrochloride (46);
(3S)-3-[5-[4-[(R)-[2-chloro-3-[(1-methylimidazol-2-yl)methoxy]phenyl]-phenyl-methyl]piperidine-1-carbonyl]-6-methyl-1-oxo-isoindolin-2-yl]piperidine-2,6-dione hydrochloride (47);
(3S)-3-[5-[4-[(R)-[2-chloro-3-[(1-methylimidazol-4-yl)methoxy]phenyl]-phenyl-methyl]piperidine-1-carbonyl]-6-methyl-1-oxo-isoindolin-2-yl]piperidine-2,6-dione hydrochloride (48);
(3S)-3-[5-[4-[(R)-[2-chloro-3-[(2-methyl-2-azaspiro[3.3]heptan-6-yl)oxy]phenyl]-phenyl-methyl]piperidine-1-carbonyl]-6-methyl-1-oxo-isoindolin-2-yl]piperidine-2,6-dione (49);
(3S)-3-[5-[4-[(R)-[2-chloro-3-[(3S)-1-(2,2,2-trifluoroethyl)pyrrolidin-3-yl]oxy-phenyl]-phenyl-methyl]piperidine-1-carbonyl]-6-methyl-1-oxo-isoindolin-2-yl]piperidine-2,6-dione (50);
3S)-3-[5-[4-[(R)-(2-chloro-3-isopropoxy-phenyl)-phenyl-methyl]piperidine-1-carbonyl]-6-methyl-1-oxo-isoindolin-2-yl]piperidine-2,6-dione (51);
(3S)-3-[5-[4-[(R)-[2-chloro-3-[(3R)-1-(2,2,2-trifluoroethyl)pyrrolidin-3-yl]oxy-phenyl]-phenyl-methyl]piperidine-1-carbonyl]-6-methyl-1-oxo-isoindolin-2-yl]piperidine-2,6-dione (52);
(3S)-3-[5-[4-[(S)-[2-chloro-3-(6,8-dihydro-5H-imidazo[1,5-a]pyrazin-7-yl)phenyl]-phenyl-methyl]piperidine-1-carbonyl]-6-methyl-1-oxo-isoindolin-2-yl]piperidine-2,6-dione (53);
(3S)-3-[5-[4-[(R)-[2-chloro-3-[(3R)-1-(2,2-difluoroethyl)pyrrolidin-3-yl]oxy-phenyl]-phenyl-methyl]piperidine-1-carbonyl]-6-methyl-1-oxo-isoindolin-2-yl]piperidine-2,6-dione (54);
(3S)-3-[5-[4-[(R)-[2-chloro-3-(cyclobutoxy)phenyl]-phenyl-methyl]piperidine-1-carbonyl]-6-methyl-1-oxo-isoindolin-2-yl]piperidine-2,6-dione (55);
N-[2-chloro-3-[[1-[2-[(3S)-2,6-dioxo-3-piperidyl]-6-methyl-1-oxo-isoindoline-5-carbonyl]-4-piperidyl]-phenyl-methyl]phenyl]-2-hydroxy-acetamide (56);
N-[2-[2-chloro-3-[(R)-[1-[2-[(3S)-2,6-dioxo-3-piperidyl]-6-methyl-1-oxo-isoindoline-5-carbonyl]-4-piperidyl]-phenyl-methyl]phenoxy]ethyl]acetamide (57);
(3S)-3-[5-[4-[(R)-[3-[(1S,3S)-3-aminocyclopentoxy]-2-chloro-phenyl]-phenyl-methyl]piperidine-1-carbonyl]-6-methyl-1-oxo-isoindolin-2-yl]piperidine-2,6-dione hydrochloride (58);
(3S)-3-[5-[4-[(R)-[3-[(2S)-2-amino-3,3-difluoro-propoxy]-2-chloro-phenyl]-phenyl-methyl]piperidine-1-carbonyl]-6-methyl-1-oxo-isoindolin-2-yl]piperidine-2,6-dione hydrochloride (59);
(3S)-3-[5-[4-[(R)-[2-chloro-3-(2-methoxyethoxy)phenyl]-phenyl-methyl]piperidine-1-carbonyl]-6-methyl-1-oxo-isoindolin-2-yl]piperidine-2,6-dione (60);
(3S)-3-[5-[4-[(R)-[3-(3-aminopropoxy)-2-chloro-phenyl]-phenyl-methyl]piperidine-1-carbonyl]-6-methyl-1-oxo-isoindolin-2-yl]piperidine-2,6-dione hydrochloride (61);
(3S)-3-[5-[4-[(R)-[3-[(2S)-2-amino-3-methyl-butoxy]-2-chloro-phenyl]-phenyl-methyl]piperidine-1-carbonyl]-6-methyl-1-oxo-isoindolin-2-yl]piperidine-2,6-dione hydrochloride (62);
(3S)-3-[5-[4-[indolin-4-yl(phenyl)methyl]piperidine-1-carbonyl]-6-methyl-1-oxo-isoindolin-2-yl]piperidine-2,6-dione hydrochloride (63);
(3S)-3-[5-[4-[(R)-[2-chloro-3-[2-[(3S)-pyrrolidin-3-yl]ethoxy]phenyl]-phenyl-methyl]piperidine-1-carbonyl]-6-methyl-1-oxo-isoindolin-2-yl]piperidine-2,6-dione 4-methylbenzenesulfonic acid (64);
(3S)-3-[5-[4-[(R)-[2-chloro-3-(methylamino)phenyl]-phenyl-methyl]piperidine-1-carbonyl]-6-methyl-1-oxo-isoindolin-2-yl]piperidine-2,6-dione (65);
(3S)-3-[5-[4-[(R)-[3-[(2S)-2-aminopropoxy]-2-chloro-phenyl]-phenyl-methyl]piperidine-1-carbonyl]-6-methyl-1-oxo-isoindolin-2-yl]piperidine-2,6-dione hydrochloride (66);
(3S)-3-[5-[4-[(R)-[3-[(2S)-3-amino-2-fluoro-propoxy]-2-chloro-phenyl]-phenyl-methyl]piperidine-1-carbonyl]-6-methyl-1-oxo-isoindolin-2-yl]piperidine-2,6-dione hydrochloride (67);
(3S)-3-[5-[4-[(R)-[3-[(3R)-3-aminopyrrolidin-1-yl]-2-chloro-phenyl]-phenyl-methyl]piperidine-1-carbonyl]-6-methyl-1-oxo-isoindolin-2-yl]piperidine-2,6-dione hydrochloride (68);
(3S)-3-[5-[4-[(R)-[3-[(1-aminocyclopropyl)methoxy]-2-chloro-phenyl]-phenyl-methyl]piperidine-1-carbonyl]-6-methyl-1-oxo-isoindolin-2-yl]piperidine-2,6-dione hydrochloride (69);
(3S)-3-[5-[4-[(R)-[2-chloro-3-[(3R)-3-hydroxypyrrolidin-1-yl]phenyl]-phenyl-methyl]piperidine-1-carbonyl]-6-methyl-1-oxo-isoindolin-2-yl]piperidine-2,6-dione (70);
(3S)-3-[5-[4-[(R)-[3-[(2R)-3-amino-2-fluoro-propoxy]-2-chloro-phenyl]-phenyl-methyl]piperidine-1-carbonyl]-6-methyl-1-oxo-isoindolin-2-yl]piperidine-2,6-dione hydrochloride (71);
(3S)-3-[5-[4-[(R)-[2-chloro-3-[(3R)-1-(2-fluoroethyl)pyrrolidin-3-yl]oxy-phenyl]-phenyl-methyl]piperidine-1-carbonyl]-6-methyl-1-oxo-isoindolin-2-yl]piperidine-2,6-dione hydrochloride (72);
(3S)-3-[5-[4-[(R)-[2-chloro-3-(1H-imidazol-5-ylmethoxy)phenyl]-phenyl-methyl]piperidine-1-carbonyl]-6-methyl-1-oxo-isoindolin-2-yl]piperidine-2,6-dione hydrochloride (73);
(3S)-3-[5-[4-[(R)-[2-chloro-3-(oxazol-5-ylmethoxy)phenyl]-phenyl-methyl]piperidine-1-carbonyl]-6-methyl-1-oxo-isoindolin-2-yl]piperidine-2,6-dione (74);
(3S)-3-[5-[4-[(R)-[2-chloro-3-(oxazol-4-ylmethoxy)phenyl]-phenyl-methyl]piperidine-1-carbonyl]-6-methyl-1-oxo-isoindolin-2-yl]piperidine-2,6-dione (75);
(3S)-3-[5-[4-[rel-(S)-indolin-4-yl(phenyl)methyl]piperidine-1-carbonyl]-6-methyl-1-oxo-isoindolin-2-yl]piperidine-2,6-dione (76);
(3S)-3-[5-[4-[rel-(R)-indolin-4-yl(phenyl)methyl]piperidine-1-carbonyl]-6-methyl-1-oxo-isoindolin-2-yl]piperidine-2,6-dione (77);
(3S)-3-[5-[4-[(R)-[3-[(2S)-2-amino-3,3,3-trifluoro-propoxy]-2-chloro-phenyl]-phenyl-methyl]piperidine-1-carbonyl]-6-methyl-1-oxo-isoindolin-2-yl]piperidine-2,6-dione (78);
(3S)-3-[5-[4-[(R)-[3-[((1r,3r)-3-aminocyclobutyl)amino]-2-chloro-phenyl]-phenyl-methyl]piperidine-1-carbonyl]-6-methyl-1-oxo-isoindolin-2-yl]piperidine-2,6-dione hydrochloride (79);
(3S)-3-[5-[4-[(R)-[3-[(1S)-2-amino-1-methyl-ethoxy]-2-chloro-phenyl]-phenyl-methyl]piperidine-1-carbonyl]-6-methyl-1-oxo-isoindolin-2-yl]piperidine-2,6-dione hydrochloride (80);
(3S)-3-[5-[4-[(R)-[3-[(2R)-2-amino-3,3-difluoro-propoxy]-2-chloro-phenyl]-phenyl-methyl]piperidine-1-carbonyl]-6-methyl-1-oxo-isoindolin-2-yl]piperidine-2,6-dione hydrochloride (81);
(3S)-3-[5-[4-[rel-(S)-[3-(2-aminoethoxy)-2-fluoro-phenyl]-phenyl-methyl]piperidine-1-carbonyl]-6-methyl-1-oxo-isoindolin-2-yl]piperidine-2,6-dione hydrochloride (82);
(3S)-3-[5-[4-[(R)-[3-(3-amino-2,2-difluoro-propoxy)-2-chloro-phenyl]-phenyl-methyl]piperidine-1-carbonyl]-6-methyl-1-oxo-isoindolin-2-yl]piperidine-2,6-dione hydrochloride (83);
(3S)-3-[5-[4-[rel-(R)-(2-chlorophenyl)-(3-pyridyl)methyl]piperidine-1-carbonyl]-6-methyl-1-oxo-isoindolin-2-yl]piperidine-2,6-dione hydrochloride (84);
(3S)-3-[5-[4-[(R)-[3-(2-amino-4,4-difluoro-butoxy)-2-chloro-phenyl]-phenyl-methyl]piperidine-1-carbonyl]-6-methyl-1-oxo-isoindolin-2-yl]piperidine-2,6-dione hydrochloride (85);
(3S)-3-[5-[4-[(R)-[3-[(2S)-2-aminobutoxy]-2-chloro-phenyl]-phenyl-methyl]piperidine-1-carbonyl]-6-methyl-1-oxo-isoindolin-2-yl]piperidine-2,6-dione hydrochloride (86);
(3S)-3-[5-[4-[(R)-[3-[(2R)-2-amino-3,3,3-trifluoro-propoxy]-2-chloro-phenyl]-phenyl-methyl]piperidine-1-carbonyl]-6-methyl-1-oxo-isoindolin-2-yl]piperidine-2,6-dione (87);
(3S)-3-[5-[4-[(R)-[2-chloro-3-(isoxazol-4-ylmethoxy)phenyl]-phenyl-methyl]piperidine-1-carbonyl]-6-methyl-1-oxo-isoindolin-2-yl]piperidine-2,6-dione (88);
(3S)-3-[5-[4-[(R)-[2-chloro-3-(isoxazol-5-ylmethoxy)phenyl]-phenyl-methyl]piperidine-1-carbonyl]-6-methyl-1-oxo-isoindolin-2-yl]piperidine-2,6-dione (89);
(3S)-3-[5-[4-[(R)-(2-chloro-3-morpholino-phenyl)-phenyl-methyl]piperidine-1-carbonyl]-6-methyl-1-oxo-isoindolin-2-yl]piperidine-2,6-dione (90);
(3S)-3-[5-[4-[(R)-[2-chloro-3-(isoxazol-3-ylmethoxy)phenyl]-phenyl-methyl]piperidine-1-carbonyl]-6-methyl-1-oxo-isoindolin-2-yl]piperidine-2,6-dione (91);
(3S)-3-[5-[4-[(R)-[2-chloro-3-(oxazol-2-ylmethoxy)phenyl]-phenyl-methyl]piperidine-1-carbonyl]-6-methyl-1-oxo-isoindolin-2-yl]piperidine-2,6-dione (92);
(3S)-3-[6-methyl-1-oxo-5-[4-[phenyl(1,2,3,4-tetrahydroquinolin-5-yl)methyl]piperidine-1-carbonyl]isoindolin-2-yl]piperidine-2,6-dione (93);
(3S)-3-[5-[4-[(R)-[2-chloro-3-(2-hydroxyethylamino)phenyl]-phenyl-methyl]piperidine-1-carbonyl]-6-methyl-1-oxo-isoindolin-2-yl]piperidine-2,6-dione (94);
(3S)-3-[5-[4-[(R)-[3-[(1R)-2-amino-1-methyl-ethoxy]-2-chloro-phenyl]-phenyl-methyl]piperidine-1-carbonyl]-6-methyl-1-oxo-isoindolin-2-yl]piperidine-2,6-dione hydrochloride (95);
(3S)-3-[5-[4-[3,4-dihydro-2H-1,4-benzoxazin-8-yl(phenyl)methyl]piperidine-1-carbonyl]-6-methyl-1-oxo-isoindolin-2-yl]piperidine-2,6-dione (96);
(3S)-3-[5-[4-[indolin-6-yl(phenyl)methyl]piperidine-1-carbonyl]-6-methyl-1-oxo-isoindolin-2-yl]piperidine-2,6-dione (97);
(3S)-3-[5-[4-[(R)-(2-chloro-3-ethoxy-phenyl)-phenyl-methyl]piperidine-1-carbonyl]-6-methyl-1-oxo-isoindolin-2-yl]piperidine-2,6-dione (98);
(3S)-3-[5-[4-[(R)-[3-(2-aminoethylamino)-2-chloro-phenyl]-phenyl-methyl]piperidine-1-carbonyl]-6-methyl-1-oxo-isoindolin-2-yl]piperidine-2,6-dione hydrochloride (99);
(3S)-3-[5-[4-[(R)-[2-chloro-3-(trideuteriomethoxy)phenyl]-phenyl-methyl]piperidine-1-carbonyl]-6-methyl-1-oxo-isoindolin-2-yl]piperidine-2,6-dione (100);
(3S)-3-[5-[4-[(R)-[2-chloro-3-(2-fluoroethoxy)phenyl]-phenyl-methyl]piperidine-1-carbonyl]-6-methyl-1-oxo-isoindolin-2-yl]piperidine-2,6-dione (101);
(3S)-3-[5-[4-[(R)-[2-chloro-3-(cyclopropoxy)phenyl]-phenyl-methyl]piperidine-1-carbonyl]-6-methyl-1-oxo-isoindolin-2-yl]piperidine-2,6-dione (102);
3-[5-[4-[(R)-(2-chlorophenyl)-phenyl-methyl]piperidine-1-carbonyl]-6-methyl-1-oxo-isoindolin-2-yl]piperidine-2,6-dione (103);
(3S)-3-[6-methyl-1-oxo-5-[4-[rel-(S)-phenyl(1,2,3,4-tetrahydroquinolin-5-yl)methyl]piperidine-1-carbonyl]isoindolin-2-yl]piperidine-2,6-dione (104);
(3S)-3-[5-[4-[(2-chloro-3-pyridyl)-phenyl-methyl]piperidine-1-carbonyl]-6-methyl-1-oxo-isoindolin-2-yl]piperidine-2,6-dione (105);
(3S)-3-[6-methyl-1-oxo-5-[4-[phenyl(2,3,4,5-tetrahydro-1H-1-benzazepin-6-yl)methyl]piperidine-1-carbonyl]isoindolin-2-yl]piperidine-2,6-dione (106);
(3S)-3-[6-methyl-1-oxo-5-[4-[phenyl(1,2,3,5-tetrahydro-4,1-benzoxazepin-6-yl)methyl]piperidine-1-carbonyl]isoindolin-2-yl]piperidine-2,6-dione (107);
(3S)-3-[5-[4-[2,3-dihydrobenzofuran-4-yl(phenyl)methyl]piperidine-1-carbonyl]-6-methyl-1-oxo-isoindolin-2-yl]piperidine-2,6-dione (108);
(3S)-3-[5-[4-[1H-indazol-4-yl(phenyl)methyl]piperidine-1-carbonyl]-6-methyl-1-oxo-isoindolin-2-yl]piperidine-2,6-dione (109);
(3S)-3-[5-[4-[(2-chloro-3-methoxy-phenyl)-deuterio-phenyl-methyl]piperidine-1-carbonyl]-6-methyl-1-oxo-isoindolin-2-yl]piperidine-2,6-dione (110);
(3S)-3-[5-[4-[[2-chloro-3-(trifluoromethoxy)phenyl]-phenyl-methyl]piperidine-1-carbonyl]-6-methyl-1-oxo-isoindolin-2-yl]piperidine-2,6-dione (111);
(3S)-3-[rel-(1S)-6-[4-[(R)-(2-chlorophenyl)-phenyl-methyl]piperidine-1-carbonyl]-1,5-dimethyl-3-oxo-isoindolin-2-yl]piperidine-2,6-dione (112);
(3S)-3-[5-[4-[rel-(S)-(2-chloro-4-fluoro-3-methoxy-phenyl)-phenyl-methyl]piperidine-1-carbonyl]-6-methyl-1-oxo-isoindolin-2-yl]piperidine-2,6-dione (113); or
(3S)-3-[5-[4-[[3-(2-aminoethoxy)-2-bromo-phenyl]-phenyl-methyl]piperidine-1-carbonyl]-6-methyl-1-oxo-isoindolin-2-yl]piperidine-2,6-dione hydrochloride (114).

11. A pharmaceutical composition comprising a compound according to claim 1 or tautomers or a pharmaceutically acceptable salt thereof; and a pharmaceutically acceptable carrier.

12. A method for the treatment of cancer, in a patient comprising administering to said patient a therapeutically effective amount of a compound, a tautomer or a pharmaceutically acceptable salt thereof according to claim 1.

13. The method according to claim 12 wherein said cancer is selected from cancer of the colon, gastric, pancreatic cancer, breast cancer, prostate cancer, lung cancer, ovarian cancer, cervical cancer, renal cancer, cancer of the head and neck, lymphoma, leukemia, and melanoma.

14. The method according to claim 12 further comprising administering to said patient a therapeutically effective amount of a second agent, wherein said second agent is selected from an antagonist of PD1/PD-L1 axis, an antagonist of CTLA4, an antagonist of LAG-3, a chemotherapeutic agent, radiation, or an anti-tumor vaccine, prior to, simultaneously with or after administration of said compound.

15. The method according to claim 26, wherein said compound decreases the level of CDK2 protein and wherein said CDK21 is the amino acid sequence encoded by SEQ ID NOs: 1 or 2.

16. The method according to claim 15, wherein said CDK2 protein level is decreased by at least 70%.
